(19) United States
(12) Patent Application Publication
(10) Pub. No.: US 2025/0243515 A1
(43) Pub. Date: Jul. 31, 2025

(54) NUCLEASES AND COMPOSITIONS, SYSTEMS, AND METHODS THEREOF

(71) Applicant: Profluent Bio Inc., Berkeley, CA (US)

(72) Inventors: Ali Madani, Berkeley, CA (US); Jeffrey A. Ruffolo, Berkeley, CA (US); Stephen Nayfach, Berkeley, CA (US); Aadyot Bhatnagar, Berkeley, CA (US); Joel Beazer, Berkeley, CA (US)

(21) Appl. No.: 18/921,949

(22) Filed: Oct. 21, 2024

Related U.S. Application Data
(63) Continuation of application No. 18/623,883, filed on Apr. 1, 2024, now Pat. No. 12,123,016.
(60) Provisional application No. 63/627,457, filed on Jan. 31, 2024.

(51) Int. Cl. C12N 15/90; C12N 9/22; C12N 15/11

(52) U.S. Cl. CPC C12N 15/907; C12N 9/22; C12N 15/11; C12N 2310/20; C12N 2800/80

(57) ABSTRACT

The present disclosure provides components, compositions, methods, and systems thereof for nucleic acid editing. Particularly, the disclosure provides engineered nucleases, fusion proteins of the engineered nucleases, systems including the engineered nucleases, and methods of using thereof.

### CROSS-REFERENCE TO RELATED APPLICATIONS

This application is a continuation of U.S. application Ser. No. 18/623,883, filed Apr. 1, 2024, which claims the benefit of U.S. Provisional Application No. 63/627,457, filed Jan. 31, 2024, the content of which is herein incorporated by reference in its entirety.

### FIELD

The present disclosure relates to components, compositions, methods, and systems thereof for nucleic acid editing. Particularly, the disclosure relates to engineered nucleases, fusion proteins of the engineered nucleases, systems including the engineered nucleases, and methods of using thereof.

### SEQUENCE LISTING STATEMENT

The content of the electronic sequence listing titled PROF_42580_203_SequenceListing.xml (Size: 841,404 bytes; and Date of Creation: Apr. 1, 2024) is herein incorporated by reference in its entirety.

### BACKGROUND

Methods for precisely and efficiently editing nucleic acid sequences, particularly in vivo, are challenging to develop but when successful enable studies of gene function and open doors to new therapies for human genetic diseases. Over the past few decades, development in the use of nucleases, including meganucleases (MNs), zinc figure nucleases (ZFNs), Transcription Activator-Like Effector Nucleases (TALENs), and Clustered Regularly Interspaced Short Palindromic Repeats (CRISPR) associated (Cas) nucleases, has enabled modification of specific DNA sequences within cells, both eukaryotic and prokaryotic. However, there is a continuing need to expand the available nucleases which are efficient, precise, and suitable for use in genetic engineering methods and therapies, particularly in eukaryotic cells and organisms.

### SUMMARY

Provided herein are engineered nucleases. In some embodiments, the engineered nucleases have an amino acid sequence with at least 75% identity to any of SEQ ID NOs: 1-304. In some embodiments, the engineered nucleases have an amino acid sequence with at least 90% identity to any of SEQ ID NOs: 1-304. In some embodiments, the engineered nucleases have an amino acid sequence of any of SEQ ID NO: 1-304.

In some embodiments, the engineered nucleases comprise an amino acid sequence having at least 75% identity to any one of SEQ ID NOs: 201, 202, 208, 212, 223, and 250. In some embodiments, the engineered nucleases have an amino acid sequence with at least 90% identity to any of SEQ ID NOs: 201, 202, 208, 212, 223, and 250. In some embodiments, the engineered nucleases comprise an amino acid sequence of any one of SEQ ID NOs: 201, 202, 208, 212, 223, and 250.

In some embodiments, the engineered nucleases have one or more amino acid substitutions configured to fully or partially catalytically inactivate the engineered nuclease.

In some embodiments, the engineered nucleases further comprise a localization sequence, a tag sequence, a protein transduction domain sequence, or a combination thereof.

Also provided herein are fusion proteins comprising an engineered nuclease as described herein and one or more effector domains. In some embodiments, the one or more effector domains are each individually selected from the group consisting of: a transcription activator, a transcription repressor, a deaminase, a polymerase, an epigenetic modifier, and a detection agent. In some embodiments, the engineered nuclease has one or more amino acid substitutions configured to fully or partially catalytically inactivate the engineered nuclease.

Further provided are nucleic acids (e.g., vectors) encoding an engineered nuclease or fusion protein as described herein.

Additionally provided are systems comprising an engineered nuclease or fusion protein as described herein. In some embodiments, the systems further comprise at least one guide RNA. In some embodiments, at least one gRNA is complexed with the engineered nuclease.

Compositions and cells comprising an engineered nuclease, a fusion protein, a nucleic acid, a vector, or a system as disclosed herein are also provided. In some embodiments, the compositions further comprise a carrier. In some embodiments, the cell is a prokaryotic cell or a eukaryotic cell. In some embodiments, the cell is a mammalian cell. In some embodiments, the cell is a human cell.

Methods of modifying a target nucleic acid are likewise provided. In some embodiments, the methods comprise contacting the target nucleic acid with an engineered nuclease, a fusion protein, a nucleic acid, a vector, or a system as described herein.

In some embodiments, the target nucleic acid is associated with a disease or disorder.

In some embodiments, the target nucleic acid encodes a gene product. In some embodiments, the target nucleic acid is a disease-associated gene.

In some embodiments, the target nucleic acid is in a cell. In some embodiments, the contacting comprises introducing into the cell. In some embodiments, the cell is in vitro or ex vivo. In some embodiments, the cell is in vivo. In some embodiments, the introducing comprises administering to a subject.

In some embodiments, the cell is in a plant. In some embodiments, the method comprises administering to a plant, plant cell, seed, fruit, plant part, or propagation material of a plant the polypeptide, fusion protein, nucleic acid, vector, or system.

In some embodiments, the methods treat a disease or disorder in a subject. In some embodiments, the methods comprise administering to the subject in need thereof an effective amount of an engineered nuclease, a fusion protein, a nucleic acid, a vector, or a system as described herein. In some embodiments, the subject is a human. In some embodiments, the target nucleic acid is a disease-associated gene. In some embodiments, the target nucleic acid encodes a gene product.

Other aspects and embodiments of the disclosure will be apparent in light of the following detailed description.

### BRIEF DESCRIPTION OF THE DRAWINGS

FIGS. 1A-1C are graphs of the editing efficiency in HEK 293 cells as determined from Sanger sequencing data and the Inference of CRISPR Edits program (ICE) for selected engineered nucleases, as disclosed herein, using the indicated guide RNAs.

FIGS. 2A-2C are graphs of the editing efficiency as shown based on indel rates for the indicated engineered nucleases using a target having an NGG PAM sequence (FIG. 2A), a NAG PAM sequence (FIG. 2B), or another PAM sequence (FIG. 2C).

FIGS. 3A-3C are graphs of the editing efficiency based on indel rates for the indicated engineered nucleases at three different target sites HEK2 (FIG. 3A), HEK3 (FIG. 3B), and CD3G_1 (FIG. 3C).

FIG. 4 is a graph of editing efficiency based on indel rates for the indicated engineered nucleases using the indicated synthetic gRNAs as compared to SEQ ID NO: 412 (dark grey) at the HEK3 target site.

FIG. 5 is a graph of the editing efficiency based on indel rates for Streptococcus pyogenes Cas9 (spCas9) using the indicated synthetic gRNAs as compared to SEQ ID NO: 412 (dark grey) at the HEK3 target site.

FIG. 6 is graphs of A to G editing with three base editors ABE8.20 (deaminase domain as in Gaudelli, N. M., et al. Nat Biotechnol 38, 892-900 (2020), ProBE1 (deaminase domain sequence: SEQ ID NO: 411), and ProBE-2 (deaminase domain sequence: SEQ ID NO: 410) using nuclease domains of SEQ ID NO: 201 (light grey) and SEQ ID NO: 223 (dark grey) having D10A mutations.

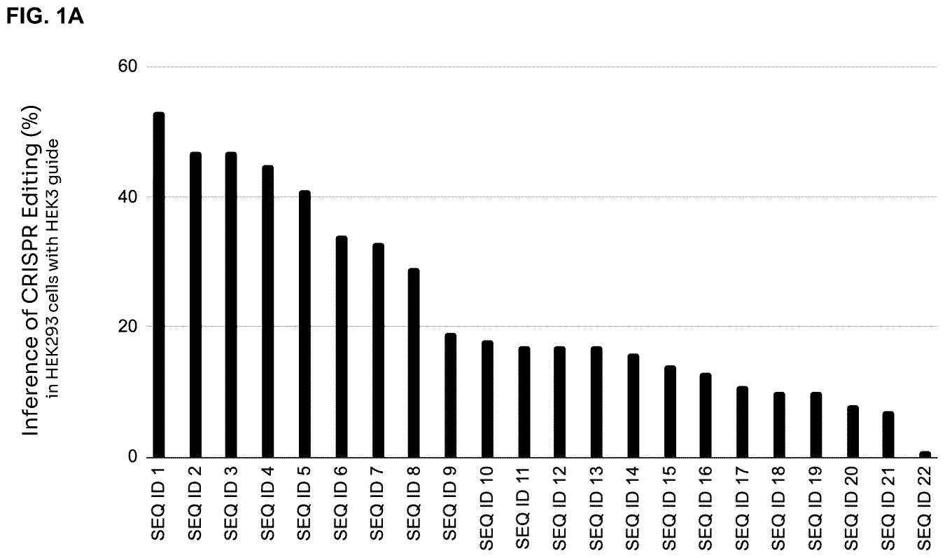

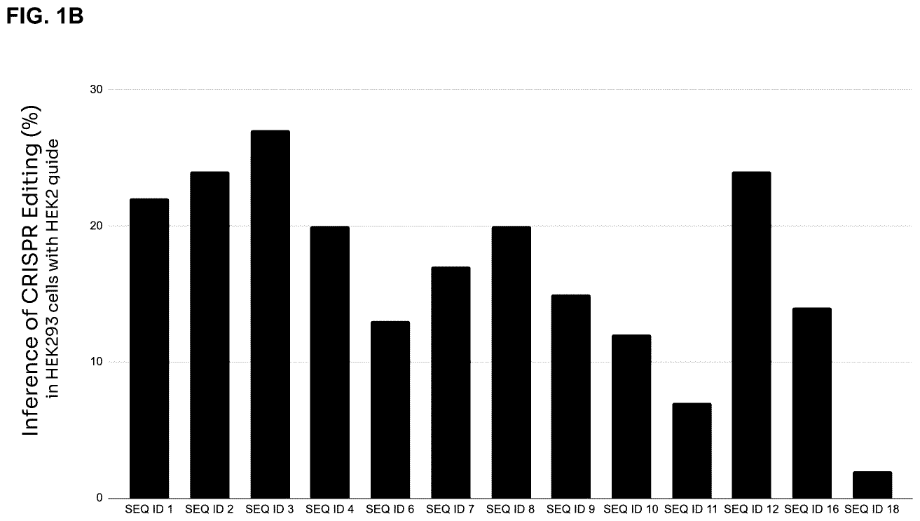

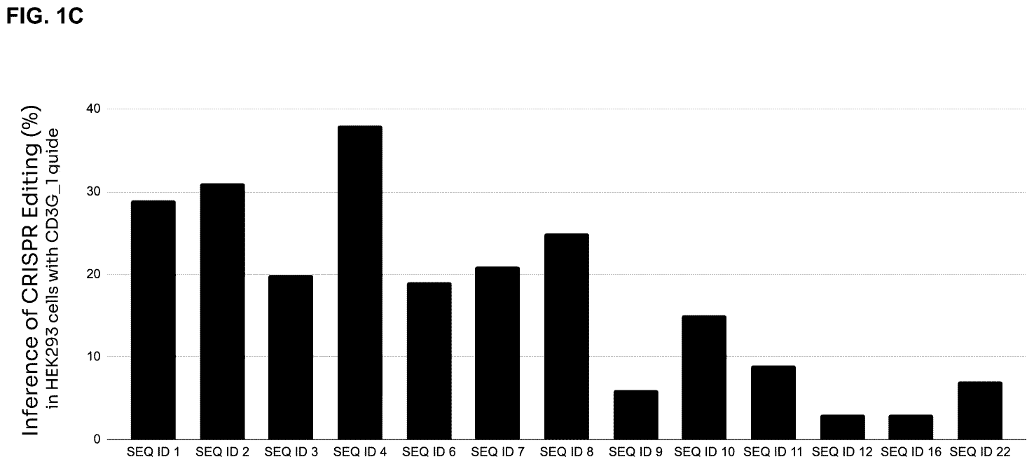

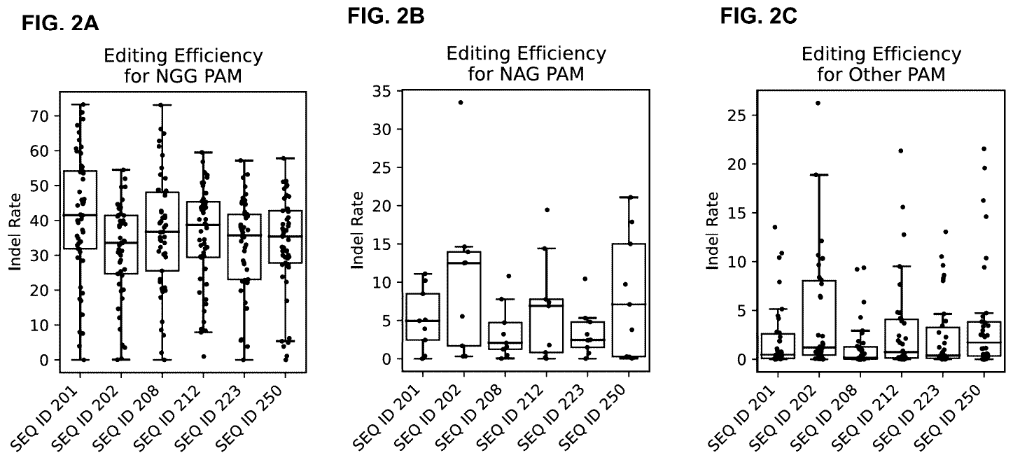

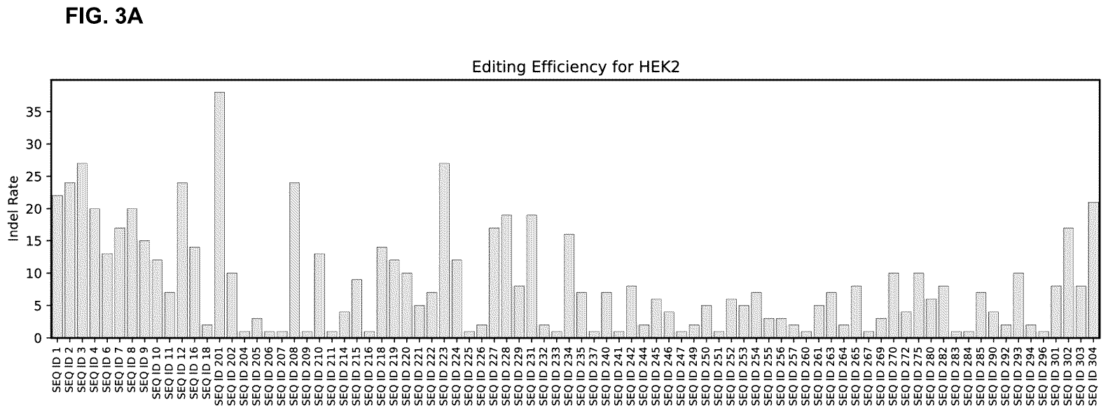

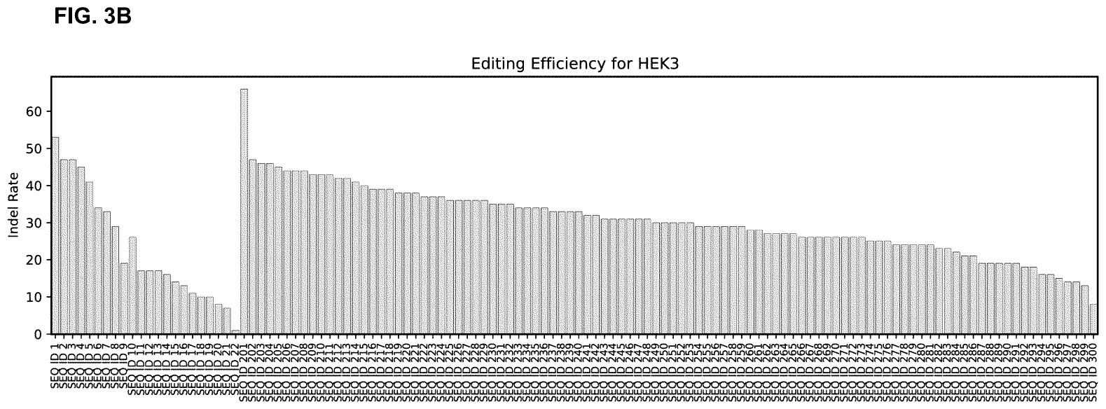

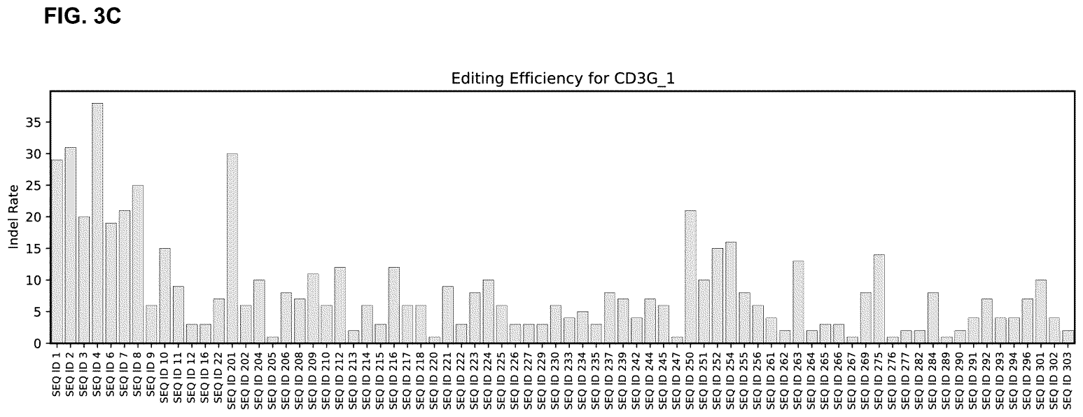

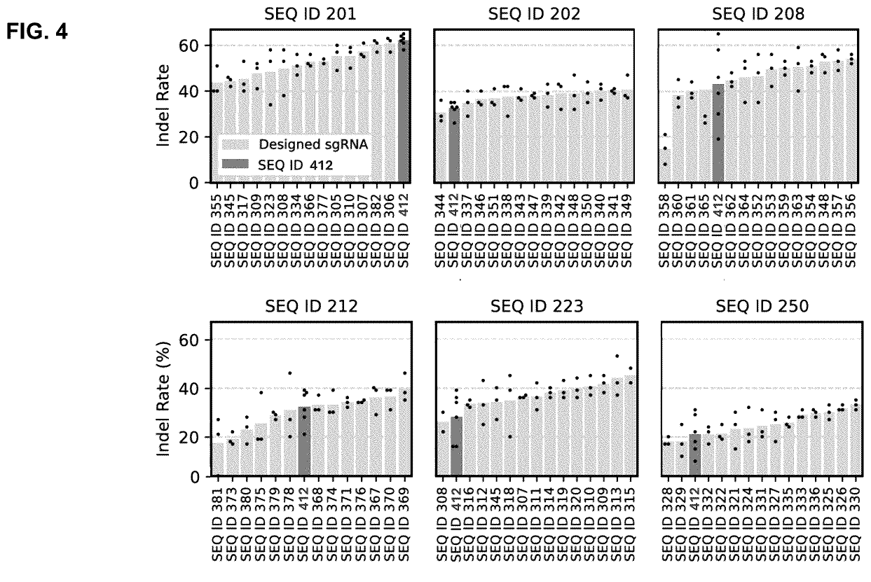

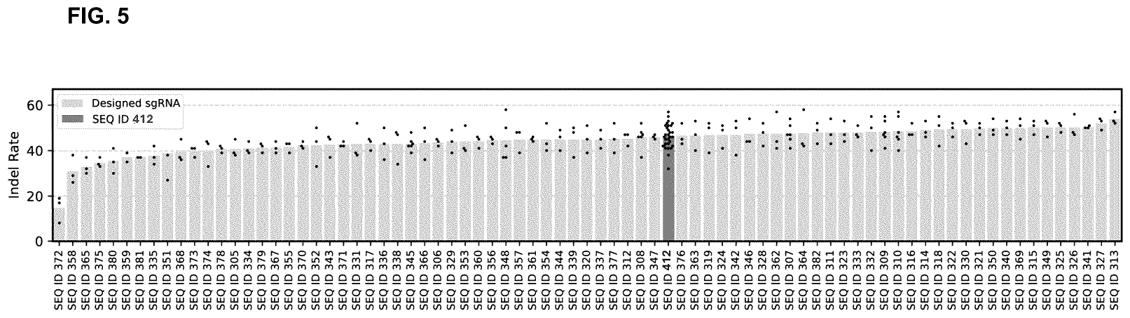

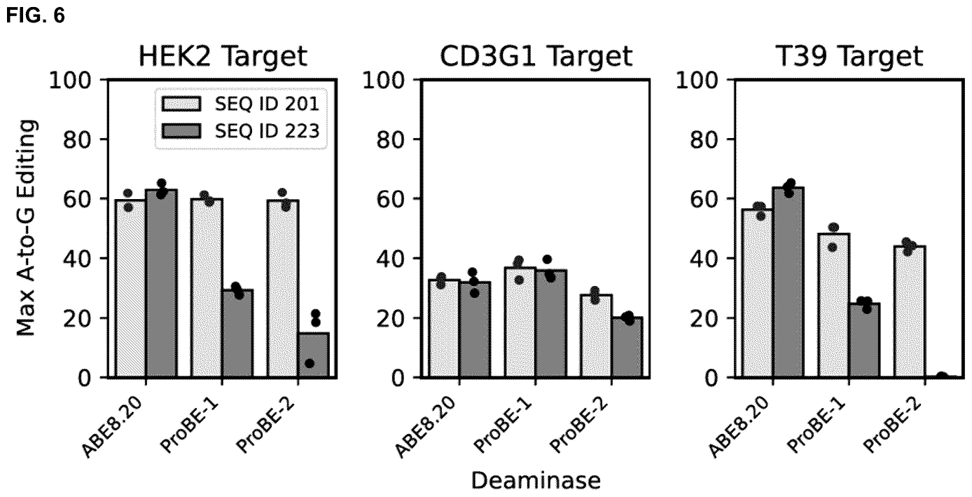

### DETAILED DESCRIPTION

The disclosed polypeptides, compositions, systems, kits, and methods include engineered nucleases useful for nucleic acid modification.

Section headings as used in this section and the entire disclosure herein are merely for organizational purposes and are not intended to be limiting.

### Definitions

The terms “comprise(s),” “include(s),” “having,” “has,” “can,” “contain(s),” and variants thereof, as used herein, are intended to be open-ended transitional phrases, terms, or words that do not preclude the possibility of additional acts or structures. As used herein, comprising a certain sequence or a certain SEQ ID NO usually implies that at least one copy of said sequence is present in recited peptide or polynucleotide. However, two or more copies are also contemplated. The singular forms “a,” “and,” and “the” include plural references unless the context clearly dictates otherwise. The present disclosure also contemplates other embodiments “comprising,” “consisting of,” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

Unless otherwise defined herein, scientific, and technical terms used in connection with the present disclosure shall have the meanings that are commonly understood by those of ordinary skill in the art. For example, any nomenclature used in connection with, and techniques of cell and tissue culture, molecular biology, microbiology, genetics and protein and nucleic acid chemistry and hybridization described herein are those that are well known and commonly used in the art. The meaning and scope of the terms should be clear; in the event, however of any latent ambiguity, definitions provided herein take precedent over any dictionary or extrinsic definition. Further, unless otherwise required by context, singular terms shall include pluralities and plural terms shall include the singular.

As used herein, “nucleic acid” or “nucleic acid sequence” refers to a polymer or oligomer of pyrimidine and/or purine bases, preferably cytosine, thymine, and uracil, and adenine and guanine, respectively (See Albert L. Lehninger, Principles of Biochemistry, 793-800 (Worth Pub. 1982)). The present technology contemplates any deoxyribonucleotide, ribonucleotide, or nucleoprotein component, and any chemical variants thereof, such as methylated, hydroxymethylated, or glycosylated forms of these bases, and the like. The polymers or oligomers may be heterogenous or homogenous in composition and may be isolated from naturally occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states. In some embodiments, a nucleic acid or nucleic acid sequence comprises other kinds of nucleic acid structures such as, for instance, a DNA/RNA helix, peptide nucleic acid (PNA), morpholino nucleic acid (see, e.g., Braasch and Corey, Biochemistry, 41 (14): 4503-4510 (2002) and U.S. Pat. No. 5,034,506), locked nucleic acid (LNA; see Wahlestedt et al., Proc. Natl. Acad. Sci. U.S.A., 97:5633-5638 (2000)), cyclohexenyl nucleic acids (see Wang, J. Am. Chem. Soc., 122:8595-8602 (2000)), and/or a ribozyme. Hence, the term “nucleic acid” or “nucleic acid sequence” may also encompass a chain comprising non-natural nucleotides, modified nucleotides, and/or non-nucleotide building blocks that can exhibit the same function as natural nucleotides (e.g., “nucleotide analogs”); further, the term “nucleic acid sequence” as used herein refers to an oligonucleotide, nucleotide or polynucleotide, and fragments or portions thereof, and to DNA or RNA of genomic or synthetic origin, which may be single or double-stranded, and represent the sense or antisense strand. The terms “nucleic acid,” “polynucleotide,” “nucleotide sequence,” and “oligonucleotide” are used interchangeably. They refer to a polymeric form of nucleotides of any length, either deoxyribonucleotides or ribonucleotides, or analogs thereof.

As used herein, “peptide,” “polypeptide,” or “protein” refer to a sequence of two or more amino acids linked by peptide bonds. The polypeptide can be natural, synthetic, or a modification or combination of natural and synthetic. The peptide or polypeptide may be modified by the addition of sugars, lipids or other moieties not included in the amino acid chain. The terms “polypeptide,” “oligopeptide,” and “peptide” are used interchangeably herein. The peptide(s) may be produced by recombinant genetic technology or chemical synthesis. The peptide(s) may be isolated and purified by any number of standard methods including, but not limited to, differential solubility (e.g., precipitation), centrifugation, chromatography (e.g., affinity, ion exchange, and size exclusion), or by any other standard techniques known in the art.

The term “amino acid” or “any amino acid” as used here refers to any and all amino acids, including naturally occurring amino acids (e.g., a-amino acids), unnatural amino acids, modified amino acids, and non-natural amino acids. It includes both D- and L-amino acids. Natural amino acids include those found in nature, such as, e.g., the 23 amino acids that combine into peptide chains to form the building-blocks of a vast array of proteins. These are primarily L stereoisomers, although a few D-amino acids occur in bacterial envelopes and some antibiotics. For the most part, the names of naturally occurring and non-naturally occurring aminoacyl residues used herein follow the naming conventions suggested by the IUPAC Commission on the Nomenclature of Organic Chemistry and the IUPAC-IUB Commission on Biochemical Nomenclature as set out in “Nomenclature of α-Amino Acids (Recommendations, 1974)” Biochemistry, 14 (2), (1975). To the extent that the names and abbreviations of amino acids and aminoacyl residues employed in this specification and appended claims differ from those suggestions, they will be made clear to the reader. Throughout the present specification, unless naturally occurring amino acids are referred to by their full name (e.g., alanine, arginine, etc.), they are designated by their conventional three-letter or single-letter abbreviations (e.g., Ala or A for alanine, Arg or R for arginine, etc.). The term “L-amino acid,” as used herein, refers to the “L” isomeric form of a peptide, and conversely the term “D-amino acid” refers to the “D” isomeric form of a peptide (e.g., Dphe, (D) Phe, D-Phe, or DF for the D isomeric form of Phenylalanine). Amino acid residues in the D isomeric form can be substituted for any L-amino acid residue, as long as the desired function is retained by the peptide.

Nucleic acid or amino acid sequence “identity,” as described herein, can be determined by comparing a nucleic acid or amino acid sequence of interest to a reference nucleic acid or amino acid sequence. A number of mathematical algorithms for obtaining the optimal alignment and calculating identity between two or more sequences are known and incorporated into a number of available software programs. Examples of such programs include CLUSTAL-W, T-Coffee, and ALIGN (for alignment of nucleic acid and amino acid sequences), BLAST programs (e.g., BLAST 2.1, BL2SEQ, and later versions thereof) and FASTA programs (e.g., FASTA3x, FAS™, and SSEARCHfor sequence alignment and sequence similarity searches). Sequence alignment algorithms also are disclosed in, for example, Altschul et al., J. Molecular Biol., 215 (3): 403-410 (1990), Beigert et al., Proc. Natl. Acad. Sci. USA, 106 (10): 3770-3775 (2009), Durbin et al., eds., Biological Sequence Analysis: Probabilistic Models of Proteins and Nucleic Acids, Cambridge University Press, Cambridge, UK (2009), Soding, Bioinformatics, 21 (7): 951-60 (2005), Altschul et al., Nucleic Acids Res., 25 (17): 3389-3402 (1997), and Gusfield, Algorithms on Strings, Trees and Sequences, Cambridge University Press, Cambridge UK (1997)).

The term “gene” refers to a nucleic acid sequence that comprises control and coding sequences necessary for the production of a gene product (e.g., an RNA having a non-coding function (e.g., a ribosomal or transfer RNA), a polypeptide, or a precursor of any of the foregoing). The RNA or polypeptide can be encoded by a full length coding sequence or by any portion of the coding sequence so long as the desired activity or function is retained. Thus, a “gene” refers to a DNA or RNA, or portion thereof, that encodes a polypeptide or an RNA chain that has functional role to play in an organism. For the purpose of this disclosure, it may be considered that genes include regions that regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites, and locus control regions.

A cell has been “genetically modified,” “transformed,” or “transfected” by exogenous DNA, e.g., a recombinant expression vector, when such DNA has been introduced inside the cell. The presence of the exogenous DNA results in permanent or transient genetic change. The transforming DNA may or may not be integrated (covalently linked) into the genome of the cell. For example, the transforming DNA may be maintained on an episomal element such as a plasmid. With respect to eukaryotic cells, a stably transformed cell is one in which the transforming DNA has become integrated into a chromosome so that it is inherited by daughter cells through chromosome replication. This stability is demonstrated by the ability of the eukaryotic cell to establish cell lines or clones that comprise a population of daughter cells containing the transforming DNA. A “clone” is a population of cells derived from a single cell or common ancestor by mitosis. A “cell line” is a clone of a primary cell that is capable of stable growth in vitro for many generations.

The terms “non-naturally occurring,” “engineered,” and “synthetic” are used interchangeably and indicate the involvement of the hand of man. The terms, when referring to nucleic acid molecules or polypeptides mean that the nucleic acid molecule or the polypeptide is at least substantially free from at least one other component with which it is naturally associated in nature and as found in nature, and/or the nucleic acid molecule or the polypeptide is associated with at least one other component with which it is not naturally associated in nature and/or that there is one or more changes in nucleic acid or amino acid sequence as compared with such sequence as it is found in nature and/or that the nucleic acid or polypeptide sequence was generated de novo, e.g., not based on or derived from any naturally occurring sequence.

A “vector” or “expression vector” is a replicon, such as plasmid, phage, virus, or cosmid, to which another DNA segment, e.g., an “insert,” may be attached or incorporated so as to bring about the replication of the attached segment in a cell.

The term “contacting” as used herein refers to bring or put in contact, to be in or come into contact. The term “contact” as used herein refers to a state or condition of touching or of immediate or local proximity.

As used herein, the terms “providing,” “administering,” and “introducing” are used interchangeably herein and refer to the placement into a cell, organism, or subject by a method or route which results in at least partial localization to a desired site. Administration may be by any appropriate route which results in delivery to a desired location in the cell, organism, or subject.

A “subject” or “patient” may be human or non-human and may include, for example, animal strains or species used as “model systems” for research purposes, such a mouse model as described herein. Likewise, a patient may include either adults or juveniles (e.g., children). Moreover, patient may mean any living organism, preferably a mammal (e.g., human or non-human) that may benefit from the administration of compositions contemplated herein. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents such as rats, mice, and guinea pigs, and the like. Examples of non-mammals include, but are not limited to, birds, fish, and the like. In one embodiment of the methods and compositions provided herein, the mammal is a human.

Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

### Nucleases

Disclosed herein are engineered nucleases. In some embodiments, the engineered nucleases comprise an amino acid sequence having at least 75% identity (e.g., at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%) to any one of SEQ ID NOs: 1-304. In some embodiments, the engineered nucleases comprise an amino acid sequence of any one of SEQ ID NOs: 1-304.

In some embodiments, the engineered nucleases comprise an amino acid sequence having at least 75% identity to any one of SEQ ID NOs: 201, 202, 208, 212, 223, and 250. In some embodiments, the engineered nucleases comprise an amino acid sequence of any one of SEQ ID NOs: 201, 202, 208, 212, 223, and 250.

Any of the engineered nucleases described herein may comprise one or more (e.g., 1, 2, 3, 4, 5, 6, 7, 8, 9, 10, 15, 20, 25, or more, etc.) amino acid substitutions as compared to SEQ ID NOs: 1-200. An amino acid “replacement” or “substitution” refers to the replacement of one amino acid at a given position or residue by another amino acid at the same position or residue within a polypeptide sequence. Amino acids are broadly grouped as “aromatic” or “aliphatic”. An aromatic amino acid includes an aromatic ring. Examples of aromatic amino acids include histidine (H or His), phenylalanine (F or Phe), tyrosine (Y or Tyr), and tryptophan (W or Trp). Non-aromatic amino acids are broadly grouped as aliphatic. Examples of aliphatic amino acids include glycine (G or Gly), alanine (A or Ala), valine (V or Val), leucine (L or Leu), isoleucine (I or Ile), methionine (M or Met), serine (S or Ser), threonine (T or Thr), cysteine (C or Cys), proline (P or Pro), glutamic acid (E or Glu), aspartic acid (A or Asp), asparagine (N or Asn), glutamine (Q or Gin), lysine (K or Lys), and arginine (R or Arg).

The amino acid replacement or substitution can be conservative, semi-conservative, or non-conservative. The phrase “conservative amino acid substitution” or “conservative mutation” refers to the replacement of one amino acid by another amino acid with a common property. A functional way to define common properties between individual amino acids is to analyze the normalized frequencies of amino acid changes between corresponding proteins of homologous organisms (Schulz and Schirmer, Principles of Protein Structure, Springer-Verlag, New York (1979)). According to such analyses, groups of amino acids may be defined where amino acids within a group exchange preferentially with each other and therefore resemble each other most in their impact on the overall protein structure (Schulz and Schirmer). Examples of conservative amino acid substitutions include substitutions of amino acids within the sub-groups described above, for example, lysine for arginine and vice versa such that a positive charge may be maintained, glutamic acid for aspartic acid and vice versa such that a negative charge may be maintained, serine for threonine such that a free-OH can be maintained, and glutamine for asparagine such that a free-NH₂ can be maintained. “Semi-conservative mutations” include amino acid substitutions of amino acids within the same groups listed above, but not within the same sub-group. For example, the substitution of aspartic acid for asparagine, or asparagine for lysine, involves amino acids within the same group, but different sub-groups. “Non-conservative mutations” involve amino acid substitutions between different groups, for example, lysine for tryptophan, or phenylalanine for serine, etc.

In some embodiments, the engineered nucleases comprise one or more amino acid substitutions configured to fully or partially catalytically inactivate the nuclease. For example, the engineered nucleases may comprise one or more amino acids substitutions in one or more of the catalytic domains (e.g., a RuvC domain, an HNH domain), resulting in full or partial inhibition of nuclease functionality. Full inhibition renders the engineered nuclease completely absent of any nuclease functionality. Partial inhibition can be a result of catalytic inactivation of one of the catalytic domains of the engineered nuclease. Such partial inhibition can remove the ability to cleave two strands of a target nucleic acid, resulting in an enzyme referred to as a nickase only capable of cleaving a single strange of a double-stranded target nucleic acid. For example, in some embodiments, the engineered nucleases have a D10A mutation.

In some embodiments, the engineered nucleases comprise an amino acid sequence having less than about 80% (e.g., less than about 75%, less than about 70%, less than about 65%, etc.) identity to the sequence of Streptococcus pyogenes Cas9 (SpCas9).

In some embodiments, the engineered nuclease further comprises a localization or signal sequence (e.g., nuclear localization sequence), a sequence tag (e.g., a tag for detection, purification, and/or monitoring expression), a protein transduction domain sequence, or a combination thereof.

In some embodiments, the engineered nuclease comprises one or more nuclear localization sequences (NLSs). The nuclear localization sequence may be appended, for example, to the N-terminus, a C-terminus, internally, or a combination thereof. In such cases when the engineered nuclease comprises two or more NLSs, the NLSs may be in tandem, separated by a linker, at either end of the protein, or one or more may be embedded in the protein.

The nuclear localization sequence may comprise any amino acid sequence known in the art to functionally tag or direct a protein for import into a cell's nucleus (e.g., for nuclear transport). Usually, a nuclear localization sequence comprises one or more positively charged amino acids, such as lysine and arginine. The NLS may be appended by a linker.

In some embodiments, the NLS is a monopartite sequence. A monopartite NLS comprises a single cluster of positively charged or basic amino acids. In some embodiments, the monopartite NLS comprises a sequence of K-K/R-X-K/R, wherein X can be any amino acid. Exemplary monopartite NLS sequences include those from the SV40 large T-antigen, c-Myc, and TUS-proteins. In some embodiments, the NLS is a bipartite sequence. Bipartite NLSs comprise two clusters of basic amino acids, separated by a spacer of about 9-12 amino acids. Exemplary bipartite NLSs include the nuclear localization sequences of nucleoplasmin, EGL-12, or bipartite SV40. In some embodiments, the NLS comprises a sequence of: KR(K/R)R (SEQ ID NOs: 383-384); K(K/R)RK (SEQ ID NOs: 385-386); (R/P)XXKR(K/R) (\hat{ }DE) (SEQ ID NOS: 387-390) or (R/P)XXKR (\hat{ }DE) (K/R) (SEQ ID NOs: 391-394) wherein (\hat{ }DE) represents any amino acid except for Asp or Glu; KRX(W/F/Y)XXAF (SEQ ID NOs: 395-397); LGKR(K/R)(W/F/Y) (SEQ ID NO: 398-399); or a bipartite sequence thereof.

The engineered nuclease may also comprise a tag (e.g., 3×FLAG tag, an HA tag, a Myc tag, a poly-histidine tag, a SNAP-tag, a CLIP-tag, and the like). The tags may be at the N-terminus, a C-terminus, or a combination thereof of the engineered nuclease. In some embodiments, the tag may be adjacent, either upstream or downstream, to a nuclear localization sequence.

In some embodiments, the engineered nuclease may be fused with one or more (e.g., two, three, four, or more) protein transduction moieties. A protein transduction moiety is a polypeptide, polynucleotide, carbohydrate, or organic or inorganic compound that facilitates traversing a lipid bilayer, micelle, cell membrane, organelle membrane, or vesicle membrane. A protein transduction moiety attached to another molecule facilitates the molecule traversing a membrane, for example going from extracellular space to intracellular space, or cytosol to within an organelle.

Accordingly in some embodiments, the engineered nuclease comprises one or more polypeptide transduction moieties. The polypeptide transduction moiety may be at a terminus of the engineered nuclease (e.g., N-terminus or C-terminus), or alternatively be inserted internally. Examples of polypeptide transduction moieties include but are not limited to a minimal undecapeptide polypeptide transduction domain (corresponding to residues 47-57 of HIV-1 TAT comprising); a polyarginine sequence comprising a number of arginines sufficient to direct entry into a cell (e.g., 3, 4, 5, 6, 7, 8, 9, 10, or 10-50 arginines); a VP22 domain (Zender et al. (2002) Cancer Gene Ther. 9(6): 489-96); a Drosophila Antennapedia protein transduction domain (Noguchi et al. (2003) Diabetes 52 (7): 1732-1737); a truncated human calcitonin peptide (Trehin et al. (2004) Pharm. Research 21:1248-1256); polylysine (Wender et al. (2000) Proc. Natl. Acad. Sci. USA 97:13003-13008); transportan, and the like.

### Fusion Proteins

The present disclosure also provides fusion proteins comprising the engineered nucleases described herein and one or more effector or functional domains. The fusion proteins are not limited by orientation or directionality of the engineered nuclease and the one or more effector domains. For example, any single effector domain may be fused to the N-terminus or C-terminus of the engineered nuclease, in any orientation, e.g., N-terminus to N-terminus, C-terminus to C-terminus, N-terminus to C-terminus, or C-terminus to N-terminus, and directly or indirectly (e.g., fused to another effector domain fused to the N-terminus).

Effector or functional domains are proteins or fragments thereof that can modify, regulate, or act as a tag for a target nucleic acid. For example, an effector domain can be used to target enzymatic activities to a nucleic acid sequence which the engineered nuclease targets (e.g., by way of a guide RNA, described elsewhere herein). In some embodiments, an effector domain is a fragment of protein that has been separated from its natural DNA binding domain and engineered to be part of a fusion protein with an engineered nuclease described herein. In some embodiments, an effector domain is a protein which normally binds to other proteins or factors for recruitment to a specific or non-specific nucleic acid.

An effector or functional domain may comprise a number of functionalities, including but not limited to, recombinase function, epigenetic modifying function (e.g., histone acetylase function, histone deacetylase function), integrase function, resolvase function, invertase function, protease function, nuclease function, DNA methyltransferase function, DNA demethylase function, transcriptional repressor function, transcriptional activator function, DNA binding protein function, transcription factor recruiting protein function, nuclear-localization signal function, DNA editing function (e.g., deaminase), degradation signaling, or any combination thereof. In some embodiments, the one or more effector or functional domains include a transcription activator, a transcription repressor, a deaminase, a polymerase (e.g., reverse transcriptase), an epigenetic modifier, a detection agent (e.g., fluorescent protein or protein tag), or a combination thereof.

In some embodiments, the fusion protein is used to modulate gene regulatory activity, such as transcriptional or translational activity. For example, the one or more effector domain comprises a transcription activator and/or repressor activity that can affect transcription upstream and downstream of coding regions, and can be used to activate or repress gene expression. In some embodiments, the one or more effector domain includes domains from transcription factors (activators, repressors, coactivators, co-repressors), silencers, and/or chromatin associated proteins and their modifiers (e.g., methylases, demethylases, acetylases and deacetylases).

In some embodiments, the one or more effector domain comprises transcriptional repressor function. Transcription repressors prevent, partially or completely, the transcription of genes near to the target site. Exemplary transcriptional repressors include, but are not limited to, KRAB-domain containing proteins, SID, and Sp1.

In some embodiments, the one or more effector domain comprises transcriptional activator function. Transcriptional activators can be generally defined as proteins, or domains thereof, that bind to specific sites on promoter DNA and bring about increased transcription of specific genes through interactions with other proteins. Exemplary transcriptional activators include, but are not limited to, VP64, p65, p53, c-Myb, GATA-1, EKLF, MyoD, E2F, dTCF, Tat, HSF1, RTA and SET7/9.

In some embodiments, a fusion protein as disclosed herein can comprise an effector domain comprising a transcriptional effector recruiting domain, or active fragment thereof. The transcriptional effector recruiting domain can recruit transcriptional activators or repressors, e.g., to the specific nucleic acid sequence which the engineered nuclease is bound to localize activators and repressors to modulate gene expression in a targeted manner.

In some embodiments, the one or more effector domain comprises DNA methyltransferase or DNA methylase function. DNA methyltransferases (DNMT's) are a family of DNA modifying proteins composed of different isomers (e.g., DNMT1, DNMT3A, and DNMT3B). Other exemplary DNA methyltransferases include SssI methylase, AluI methylase, HaeIII methylase, HhaI methylase, and HpaII methylase. Their main mechanism of action is addition of a methyl group to the fifth carbon of a cytosine residue (5mc) located adjacent to a guanine residue. DNA demethylation can be mediated by at least three enzyme families: (i) the ten-eleven translocation (TET) family, mediating the conversion of 5mC into 5hmC; (ii) the AID/APOBEC family, acting as mediators of 5mC or 5hmC deamination; and (iii) the BER (base excision repair)glycosylase family involved in DNA repair.

In some embodiments, the one or more effector domain modifies epigenetic signals and thereby modify gene regulation, for example by promoting histone acetylase and histone deacetylase activity. The term “epigenetic modifier,” as used herein, refers to a protein or catalytic domain thereof having enzymatic activity that results in the epigenetic modification of DNA, for example, chromosomal DNA. Epigenetic modifications include, but are not limited to, histone modifications including methylation and demethylation (e.g., mono-, di- and tri-methylation), histone acetylation and deacetylation, as well as histone ubiquitylation, phosphorylation, and sumoylation.

Histone acetylation and deacetylation are the processes by which the lysine residues within the N-terminal tail protruding from the histone core of the nucleosome are acetylated and deacetylated as part of gene regulation. These reactions are typically catalyzed by enzymes with histone acetyltransferase (HAT) or histone deacetylase (HDAC) activity. Histone acetyltransferases include GNAT family proteins (e.g., Gcn5, Gcn5L, p300/CREB-binding protein associated factor (PCAF), Elp3, HPA2 and HAT1) and MYST family proteins (e.g., Sas3, essential SAS-related acetyltransferase (Esa1), Sas2, Tip60, MOF, MOZ, MORF, and HBO1). Histone deacetylases fall into four classes. Class I includes HDACs 1, 2, 3, and 8. Class II is divided into two subgroups, Class IIA and Class IIB. Class IIA includes HDACs 4, 5, 7, and 9 while Class IIB includes HDACs 6 and 10. Class III contains the Sirtuins and Class IV contains only HDAC11. Classes of HDAC proteins are divided and grouped together based on the comparison to the sequence homologies of Rpd3, Hos1 and Hos2 for Class I HDACs, HDA1 and Hos3 for the Class II HDACs and the sirtuins for Class III HDACs.

The site-specific methylation and demethylation of histone residues are catalyzed by methyltransferases and demethylases, respectively. Histone methylases transfer methyl groups to amino acids (e.g., lysine and arginine) of histone proteins, ultimately effecting transcription of genes. Methylases include SET1, MLL, SMYD3, G9a, GLP, EZH2, and SETDB1. Histone demethylases catalyze the removal of methyl marks from histones, an activity associated with transcriptional regulation and DNA damage repair. Demethylases include, for example, KDM1A, KDM1B, KDM2A, KDM2B, UTX, UTY, Jumonji C (JmJC) domain-containing demethylases, and GSK-J4.

In some embodiments, the one or more effector domain comprises recombinase activity. A recombinase is a site-specific enzyme that mediates the recombination of DNA between recombinase recognition sequences, which results in the excision, integration, inversion, or exchange (e.g., translocation) of DNA fragments between the recombinase recognition sequences. Recombinases can be classified into two distinct families: serine recombinases (e.g., resolvases and invertases) and tyrosine recombinases (e.g., integrases). Examples of serine recombinases include, without limitation, Hin, Gin, Tn3 (also known as TnpR), β-six, CinH, ParA, γδ, Bxb1, ϕC31, TP901, TG1, ϕBT1, R4, ϕRV1, ϕFC1, MR11, A118, U153, and gp29. Examples of tyrosine recombinases include, without limitation, Cre, FLP, R, Lambda, HK101, HK022, and pSAM2.

In some embodiments, the one or more effector domain comprises DNA editing function (e.g., deaminase, DNA repair activity, DNA damage activity, dismutase activity, alkylation activity, depurination activity, oxidation activity, pyrimidine dimer forming activity, polymerase activity (e.g., reverse transcriptase), ligase activity, helicase activity, photolyase activity or glycosylase activity). In some embodiments, the one or more effector domain comprises a reverse transcriptase. In some embodiments, the one or more effector domain comprises a deaminase, e.g., a cytosine deaminase or an adenine deaminase.

Kinases, phosphatases, and other proteins that modify or regulate other polypeptides involved in gene regulation are also useful as effector domains. Such modifiers are often involved in switching on or off transcription mediated by, for example, hormones. Other useful domains for regulating gene expression can also be obtained from the gene products of oncogenes (e.g., myc, jun, fos, myb, max, mad, rel, ets, bcl, myb, mos family members) and their associated factors and modifiers.

In some embodiments, the one or more effector domain comprises an integrase. Integrases allow for the insertion of nucleic acids, for example, into a host genome (mammalian, human, mouse, rat, monkey, frog, fish, plant (including crop plants and experimental plants like Arabidopsis), laboratory or biomedical cell lines or primary cell cultures, C. elegans, fly (Drosophila), etc.). Integrases are found in a retrovirus such as HIV (human immunodeficiency virus) and lambda integrase.

In some embodiments, the one or more effector domain comprises transposase functionality. Transposases are enzymes that bind to the end of a transposon and catalyze its movement by a cut and paste mechanism or a replicative transposition mechanism. Exemplary transposases include, but are not limited to, Tc1 transposase, Mos1 transposase, Tn5 transposase, and Mu transposase.

In some embodiments, the one or more effector domain comprises invertase activity. Invertase activity can be used to alter genome structure by swapping the orientation of a DNA fragment.

In some embodiments, the one or more effector domain comprises resolvase activity. Resolvases are site-specific recombinases that function to excise (as a circle) a segment of DNA contained between two recombination sites (called res) and include, for example, Ruv C resolvase, Holiday junction resolvase Hjc, Tn3 and γδ resolvase.

In some embodiments, the one or more effector domain comprises a peptide or polypeptide sequence responsive to a ligand, such as a hormone receptor ligand binding domain, including, for example, the ligand binding domains of the estrogen receptor, the glucocorticosteroid receptor, and the like. Such effector domains can be used to act as “gene switches,” and be regulated by inducers, such as small molecule or protein ligands, specific for the ligand binding domain.

In some embodiments, the one or more effector domain comprises sequences or domains of polypeptides that mediate direct or indirect protein-protein interactions, including, for example, a leucine zipper domain, a STAT protein N terminal domain, and/or an FK506 binding protein.

In some embodiments, the activity mediated by the one or more effector domain is a non-biological activity, such as a fluorescence activity (e.g., fluorescent proteins), luminescence activity (e.g., a luminescent protein or enzyme which results in luminescence when interacting with a substrate (e.g., luciferase)), or binding activity, such as those mediated by maltose binding protein (“MBP”), glutathione S transferase (GST), hexahistidine, c-myc, and the FLAG epitope, for facilitating detection, purification, monitoring expression, and/or monitoring cellular and subcellular localization of the engineered nuclease to which the effector domain is appended. In such embodiments, the fusion proteins can also be used as a diagnostic reagent, for example, to detect mutations in gene sequences, to purify restriction fragments from a solution, or to visualize nucleic acids.

In some embodiments, the effector domain facilitates temporal modulation of the fusion protein, and accordingly the engineered nuclease. Thus, in some embodiments, the effector domain is a degron. Degrons may be ubiquitin-independent degrons, not necessary for the polyubiquitination of their protein. Alternatively, ubiquitin-dependent degrons are implicated in the polyubiquitination process for targeting a protein to the proteasome. For example, the effector domain may comprise a truncated geminin protein. Geminin is a direct substrate of E3 ubiquitin ligase complex APC/Cdh1 and is actively ubiquitinated in the M/G1 phase. Thus, degradation of the fusion protein will be promoted during the M/G1 phase thereby restricting activity of the fusion protein to largely the G2/S phase.

The effector domains described herein are illustrative and merely provide the skilled artisan with examples of effectors that can be used in combination with the engineered nucleases, systems and methods described herein.

In some embodiments, the one or more effector or functional domain and the engineered nuclease are covalently linked in a single amino acid chain through a linker. The linker may have any of a variety of amino acid sequences. Proteins can be joined by a linker polypeptide, generally of a flexible nature, although other chemical linkages are not excluded. Suitable linkers include polypeptides of between 1 amino acids and 100 amino acids in length, 4 amino acids and 40 amino acids in length, or between 4 amino acids and 25 amino acids in length. The linking peptides may have virtually any amino acid sequence, bearing in mind that the preferred linkers will have a sequence that results in a generally flexible peptide. Small amino acids, such as glycine and alanine, are useful in creating a flexible peptide linker. A variety of different linkers are considered suitable for use, including but not limited to, glycine-serine polymers, glycine-alanine polymers, and alanine-serine polymers. Such fusion proteins can be expressed recombinantly from a single nucleic acid encoding the amino acid chain.

Alternatively, the one or more effector domain and the engineered nuclease may be individually fused to one half of a binding pair (e.g., from a recruitment system) and, when introduced into the same system or location, the one or more effector domain and the engineered nuclease form a protein conjugate through the recruitment system. The recruitment system can comprise any binding pair. For example, the recruitment system may comprise an aptamer and an aptamer binding protein. The recruitment system may be a so-called split system. Split systems include two or more polypeptide chains that reassemble into an operable fusion protein or protein conjugate upon association of the two binding partners. Split systems include, but are not limited to, intein, MS2, or SunTag based systems.

In some embodiments, the aptamer sequence is a nucleic acid (e.g., RNA aptamer) sequence. In some embodiments, the guide RNA also comprises a sequence of one or more RNA aptamers, or distinct RNA secondary structures or sequences that can recruit and bind another molecular species, an adaptor molecule, such as a nucleic acid or protein. Any RNA aptamer/aptamer binding protein pair known may be selected and used in connection with the present disclosure (see, e.g., Jayasena, S. D., Clinical Chemistry. 45(9): p. 1628-1650, (1999); Gelinas, et al., Current Opinion in Structural Biology 36: p. 122-132, (2016); and Hasegawa, H., Molecules, 21 (4): p. 421 (2016), incorporated herein by reference).

In some embodiments, the aptamer sequence is a peptide aptamer sequence. In some embodiments, the engineered nuclease comprises the peptide aptamer sequence and the effector domain comprises the peptide aptamer binding protein. In some embodiments, the effector domain comprises the peptide aptamer sequence and the engineered nuclease comprises the peptide aptamer binding protein. The peptide aptamer sequence or peptide aptamer binding protein may be fused in any orientation (e.g., N-terminus to C-terminus, C-terminus to N-terminus, N-terminus to N-terminus). The peptide aptamer sequence or peptide aptamer binding protein may be fused by a linker region. Suitable linker regions are known in the art. The linker may be flexible or configured to allow the functionality and association with the DNA or other proteins with decreased steric hindrance. The linker sequences may provide an unstructured or linear region of the polypeptide, for example, with the inclusion of one or more glycine and/or serine residues. The linker sequences can be at least about 2, 3, 4, 5, 6, 7, 8, 9, 10 or more amino acids in length.

The peptide aptamers can be naturally occurring or synthetic peptides that are specifically recognized by an affinity agent. Such aptamers include, but are not limited to, a c-Myc affinity tag, an HA affinity tag, a His affinity tag, an S affinity tag, a methionine-His affinity tag, an RGD-His affinity tag, a 7×His tag, a FLAG octapeptide, a strep tag or strep tag II, a V5 tag, or a VSV-G epitope. Corresponding aptamer binding proteins are well-known in the art and include, for example, primary antibodies, biotin, affimers, single domain antibodies, and antibody mimetics.

Any of the effector domains and engineered nucleases disclosed herein may further comprise one or more proteins, polypeptides (e.g., protein domain sequences), or peptides. For example, the engineered nucleases and/or fusion proteins disclosed herein may be fused to another protein or protein domain that provides for tagging or visualization. The one or more proteins, polypeptides (e.g., protein domain sequences), or peptides may be appended at an N-terminus, a C-terminus, internally, or a combination thereof. The one or more proteins, polypeptides (e.g., protein domain sequences), or peptides may be fused in any orientation in relationship to the disclosed protein. The one or more proteins, polypeptides (e.g., protein domain sequences), or peptides may be fused via a linker, as described above.

### Systems and Compositions

Disclosed herein are systems and compositions that comprise an engineered nuclease or fusion protein as described herein, or one or more nucleic acids encoding the engineered nuclease or fusion protein. In some embodiments, the engineered nuclease and the one or more effector domains are provided in the system as separate polypeptides or nucleic acid(s) encoding thereof, in which each is linked to a half of a binding pair. Descriptions of the engineered nucleases, effector domains, and fusion proteins provided above are equally applicable to the systems and compositions.

In some embodiments, the compositions or systems further comprise at least one guide RNA (gRNA) or one or more nucleic acids comprising a sequence encoding the least one gRNA. In instances when the composition or system comprises more than one gRNA, each may be encoded on the same or different nucleic acid as the other gRNA, together or separate from the engineered nuclease, fusion protein, or effector domain. For example, the system and compositions may comprise a first nucleic acid encoding the engineered nuclease, fusion protein, or effector domain and a second nucleic acid encoding the gRNA. In one alternative, the system and compositions may comprise a single nucleic acid encoding the engineered nuclease, fusion protein, or effector domain and one or more of the at one gRNA. In some embodiments, the at least one gRNA is provided in a ribonucleoprotein (RNP) complex with the engineered nuclease or fusion protein.

The gRNA may contain separate crRNA and tracrRNA sequences (a dual guide RNA), have the crRNA and tracrRNA fused by a flexible linker, or be a single guide RNA, sgRNA. The terms “gRNA,” “guide RNA,” and “gRNA” may be used interchangeably throughout to represent any of the form of gRNA.

The “guide sequence” refers to the sequence that hybridizes to (complementary to, partially or completely) a target nucleic acid sequence (e.g., the genome in a host cell) and therefore determines the sequence specificity of the gRNA. The portion of the gRNA that hybridizes to the target nucleic acid (a target site) is generally between 10-40 nucleotides in length, but can be longer based on the specific target. gRNAs or sgRNA(s) used in the present disclosure can be between about 5 and 100 nucleotides long, or longer. The gRNA may be a non-naturally occurring or engineered gRNA.

In some embodiments, the guide sequence and scaffold sequence are separate. In such embodiments, the guide sequence is appended to an additional sequence that is complementary to a portion of the scaffold sequence and functions to hybridize with a portion of the scaffold sequence.

In some embodiments, the guide sequence is fused to a scaffold sequence (e.g., a tracrRNA). Such a chimeric gRNA is referred to as a single guide RNA (sgRNA). Exemplary scaffold sequences will be evident to one of skill in the art and can be found, for example, in Jinek, et al. Science (2012) 337 (6096): 816-821, and Ran, et al. Nature Protocols (2013) 8:2281-2308, incorporated herein by reference in their entireties.

The gRNA may comprise a scaffold sequence of SEQ ID NO: 412, represented as DNA. In gRNA sequence all thymidines are uridines. In some embodiments, the gRNA may comprise a sequence having one or more nucleotide substitutions, insertions, or deletions as compared to SEQ ID NO: 412. In some embodiments, the gRNA comprises one or more nucleotide substitutions at positions A12, T17, A37, T44, A45, T46, and G69 in reference to SEQ ID NO: 412. In select embodiments, the gRNA comprises one or more of the following substitutions: A12G, T17C, A37T, T44A, A45C/T, T46A/C, and G69T in reference to SEQ ID NO: 412. In some embodiments, the gRNA comprises an insertion following position 36 and/or an insertion following position 61, in reference to SEQ ID NO: 412. In select embodiments, the gRNA comprises an insertion of a thymidine following position 36 and/or an insertion of adenosine, guanidine, or cytidine following position 61. In some embodiments, the gRNA comprises one or more nucleotide substitutions at positions A12, T17, A37, T44, A45, T46, and G69 and an insertion following position 36 and/or an insertion following position 61, in reference to SEQ ID NO: 412. In some embodiments, the gRNA comprises a sequence as in any of SEQ ID NO: 305-382.

The target sequence may or may not be flanked by a protospacer adjacent motif (PAM) sequence. In some embodiments, the target nucleic acid is flanked by a protospacer adjacent motif (PAM). A PAM site is a nucleotide sequence in proximity to a target sequence. For example, PAM may be a DNA sequence immediately following the DNA sequence targeted by the CRISPR-Cas system. PAM sequences are well-known in the art. Non-limiting examples of the PAM sequences include: CC, CA, AG, GT, TA, AC, CA, GC, CG, GG, CT, TG, GA, AGG, TGG, T-rich PAMs (such as TTT, TTG, TTC, etc.), NGG, NGA, NAG, and NGGNG, where “N” is any nucleotide.

In certain embodiments, the disclosed nucleases cleave a target sequence if an appropriate PAM is present. A PAM can be 5′ or 3′ of a target sequence. A PAM can be upstream or downstream of a target sequence. In one embodiment, the target sequence is immediately flanked on the 3′ end by a PAM sequence. A PAM can be 1, 2, 3, 4, 5, 6, 7, 8, 9, 10 or more nucleotides in length. In certain embodiments, a PAM is between 2-6 nucleotides in length. The target sequence may or may not be located adjacent to a PAM sequence (e.g., PAM sequence located immediately 3′ of the target sequence).

“Complementarity” refers to the ability of a nucleic acid to form hydrogen bond(s) with another nucleic acid sequence by either traditional Watson-Crick or other non-traditional types. A percent complementarity indicates the percentage of residues in a nucleic acid molecule, which can form hydrogen bonds (e.g., Watson-Crick base pairing) with a second nucleic acid sequence. Full complementarity is not necessarily required, provided there is sufficient complementarity to cause hybridization. There may be mismatches distal from the PAM.

To facilitate gRNA design, many computational tools have been developed (See Prykhozhij et al. (PLOS ONE, 10(3): (2015)); Zhu et al. (PLOS ONE, 9(9) (2014)); Xiao et al. (Bioinformatics. Jan. 21 (2014)); Heigwer et al. (Nat Methods, 11(2): 122-123 (2014)). Methods and tools for guide RNA design are discussed by Zhu (Frontiers in Biology, 10 (4) pp. 289-296 (2015)), which is incorporated by reference herein. Additionally, there are many publicly available software tools that can be used to facilitate the design of sgRNA(s); including but not limited to, Genscript Interactive CRISPR gRNA Design Tool, WU-CRISPR, and Broad Institute GPP sgRNA Designer. There are also publicly available pre-designed gRNA sequences to target many genes and locations within the genomes of many species (human, mouse, rat, zebrafish, C. elegans), including but not limited to, IDT DNA Predesigned Alt-R CRISPR-Cas9 guide RNAs, Addgene Validated gRNA Target Sequences, and GenScript Genome-wide gRNA databases.

In some embodiments, the compositions and systems may further comprise one or more additional genome engineering tools. For example, the compositions may further comprise nucleases, such as zinc finger nucleases (ZFNs) and/or transcription activator like effector nucleases (TALENs); transcriptional activators, transcriptional repressors, histone-modifying proteins, integrases, recombinases, and the like.

The compositions or systems may further comprise an excipient or carrier. Excipients and carriers may include any and all solvents, dispersion media, coatings, antibacterial and antifungal agents, isotonic and absorption delaying agents. Some examples of materials which can serve as excipients and/or carriers are sugars including, but not limited to, lactose, glucose and sucrose; starches including, but not limited to, corn starch and potato starch; cellulose and its derivatives including, but not limited to, sodium carboxymethyl cellulose, ethyl cellulose and cellulose acetate; powdered tragacanth; malt; gelatin; talc; excipients including, but not limited to, cocoa butter and suppository waxes; oils including, but not limited to, peanut oil, cottonseed oil, safflower oil, sesame oil, olive oil, corn oil and soybean oil; glycols; including propylene glycol; esters including, but not limited to, ethyl oleate and ethyl laurate; agar; buffering agents including, but not limited to, magnesium hydroxide and aluminum hydroxide; alginic acid; pyrogen-free water; isotonic saline; Ringer's solution; ethyl alcohol, and phosphate buffer solutions, as well as other non-toxic compatible lubricants including, but not limited to, sodium lauryl sulfate and magnesium stearate, as well as coloring agents, releasing agents, coating agents, sweetening, flavoring and perfuming agents, preservatives and antioxidants. The compositions of the present invention and methods for their preparation will be readily apparent to those skilled in the art. Techniques and formulations may be found, for example, in Remington's Pharmaceutical Sciences, 19th Edition (Mack Publishing Company, 1995).

In some embodiments, the excipient or carrier is pharmaceutically acceptable. Pharmaceutically acceptable carriers, including buffers, are well known in the art, and may comprise phosphate, citrate, and other organic acids; antioxidants including ascorbic acid and methionine; preservatives; low molecular weight polypeptides; proteins, such as serum albumin, gelatin, or immunoglobulins; amino acids; hydrophobic polymers; monosaccharides; disaccharides; and other carbohydrates; metal complexes; and/or non-ionic surfactants. See, e.g., Remington: The Science and Practice of Pharmacy 20th Ed. (2000) Lippincott Williams and Wilkins, Ed. K. E. Hoover.

The carrier may include a delivery vehicle. Delivery vehicles such as nanoparticle- and lipid-based delivery systems can be used. Exemplary delivery vehicles include, but are not limited to, microparticle compositions comprising a variety of polymers, liposomes or lipid nanoparticles, viral vectors, ribonucleoprotein (RNP) complexes, and the like.

Microparticles can include, but are not limited to, liposomes, nanoparticles, microspheres, nanospheres, microcapsules, and nanocapsules. In some cases, microparticle can include one or more of the following: a poly(lactide-co-glycolide), aliphatic polyesters including, but not limited to, poly-glycolic acid and poly-lactic acid, hyaluronic acid, modified polysaccharides, chitosan, cellulose, dextran, polyurethanes, polyacrylic acids, pseudo-poly(amino acids), polyhydroxybutyrate-related copolymers, polyanhydrides, polymethylmethacrylate, poly(ethylene oxide), lecithin, lipids, and phospholipids, in any combination thereof.

In some embodiments, a liposome or lipid nanoparticle encapsulates the disclosed systems, nucleic acids, or proteins (e.g., engineered nucleases or fusion proteins thereof). Methods of making lipid compositions include, for example, lipid film hydration, optionally coupled with sonication or extrusion, solvent evaporation (e.g., ethanol injection, ether injection, or reverse phase evaporation), solvent-diffusion method, hot homogenization process, detergent removal methods, or combinations thereof. Any naturally occurring or synthetic vesicle forming lipid or combinations thereof can be used, including for example, di-aliphatic chain lipids, such as phospholipids; diglycerides; di-aliphatic glycolipids; single lipids such as sphingomyelin or glycosphingolipid; steroidal lipids; hydrophilic polymer derivatized lipids; or mixtures thereof. Liposome and lipid nanoparticle compositions of the disclosure may include one or more cationic and/or ionizable lipids, phospholipids, neutral or non-cationic lipids, polyethyleneglycol (PEG)-lipid conjugates, and/or sterols. In some embodiments, the lipid nanoparticle comprises a cationic lipid and/or ionizable lipid, a neutral or non-cationic lipid, and cholesterol.

The liposomes and lipid nanoparticles described herein may also include other components typically used in the formation of vesicles (e.g., for stabilization). Examples of such other components includes, without being limited thereto, fatty alcohols, fatty acids, and/or any other pharmaceutically acceptable excipients which may affect the surface charge, the membrane fluidity and assist in the incorporation of the lipid into the lipid assembly.

The liposome and lipid nanoparticle compositions of the disclosure can also be targeting compositions, e.g., contain one or more targeting moieties or biodistribution modifiers on the surface. A targeting moiety can be any agent that is capable of specifically binding or interacting with a desired target and are generally known in the art, for example ligands such as folic acid, proteins, antibody or antibody fragments, and the like).

The phrase “pharmaceutically acceptable,” as used in connection with the present disclosure, refers to molecular entities and other ingredients of such compositions that are physiologically tolerable and do not typically produce untoward reactions when administered to a subject (e.g., a mammal, a human). Preferably, as used herein, the term “pharmaceutically acceptable” means approved by a regulatory agency of the federal or a state government or listed in the U.S. Pharmacopeia or other generally recognized pharmacopeia for use in mammals, and more particularly in humans. “Acceptable” means that the carrier is compatible with the composition (e.g., the nucleic acids, vectors, cells, proteins, or polypeptides) and does not negatively affect the subject to which the composition(s) are administered. Any of the compositions to be used in the present methods can comprise pharmaceutically acceptable carriers, excipients, or stabilizers in the form of lyophilized formations or aqueous solutions.

### Nucleic Acids

Also disclosed herein are nucleic acids encoding the engineered nucleases or fusion proteins as described herein. The nucleic acids may be DNA, RNA, or combinations thereof. In some embodiments, the nucleic acids comprise one or more messenger RNAs, one or more vectors, or any combination thereof.

In certain embodiments, the nucleic acids are engineered for codon-optimization. It will be appreciated altering codons to those most frequently used in the cells or subject of interest allows for maximum expression. Such modified nucleic acid sequences are commonly described in the art as “codon-optimized.” In some embodiments, the nucleic acid sequence is considered codon-optimized if at least about 60% (e.g., about 65%, about 70%, about 75%, about 80%, about 85%, about 90%, about 95%, or about 98%) of the codons encoded therein are preferred codons to the subject of interest.

The present disclosure also provides for DNA segments encoding the engineered nucleases or fusion proteins disclosed herein, vectors containing these segments, and cells containing the vectors. The vectors may be used to propagate the DNA segment in an appropriate cell and/or to allow expression from the segment (e.g., an expression vector). The person of ordinary skill in the art would be aware of the various vectors available for propagation and expression of a nucleic acid sequence.

The present disclosure further provides engineered, non-naturally occurring vectors and vector systems, which can encode the engineered nucleases, fusion proteins, or one or more or all of the components of the systems or compositions, as disclosed herein. The vector(s) can be introduced into a cell that is capable of expressing the polypeptide encoded thereby, including any suitable prokaryotic or eukaryotic cell.

The vectors of the present disclosure may be delivered to a eukaryotic cell. Modification of the eukaryotic cells via the present system can take place in a cell culture, where the method comprises isolating the eukaryotic cell from a subject prior to the modification. In some embodiments, the method further comprises returning said eukaryotic cell and/or cells derived therefrom to the subject.

Viral and non-viral based gene transfer methods can be used to introduce nucleic acids encoding components of the present system into cells, tissues, or a subject. Such methods can be used to administer nucleic acids encoding components of the present system to cells in culture, or in a host organism. Non-viral vector delivery systems include DNA plasmids, cosmids, RNA (e.g., a transcript of a vector described herein), a nucleic acid, and a nucleic acid complexed with a delivery vehicle. Viral vector delivery systems include DNA and RNA viruses, which have either episomal or integrated genomes after delivery to the cell. Viral vectors include, for example, retroviral, lentiviral, adenoviral, adeno-associated and herpes simplex viral vectors.

In certain embodiments, plasmids that are non-replicative, or plasmids that can be cured by high temperature may be used, such that any or all of the necessary components of the system may be removed from the cells under certain conditions. For example, this may allow for DNA integration by transforming bacteria of interest, but then being left with engineered strains that have no memory of the plasmids or vectors used for the integration.

Drug selection strategies may be adopted by positively selecting for cells that underwent DNA integration. A donor nucleic acid may contain one or more drug-selectable markers within the cargo. Then presuming that the original donor plasmid is removed, drug selection may be used to enrich for integrated clones. Colony screenings may be used to isolate clonal events.

A variety of viral constructs may be used to deliver the engineered nucleases, fusion proteins, or one or more or all of the components of the system or compositions to the targeted cells and/or a subject. Nonlimiting examples of such recombinant viruses include recombinant adeno-associated virus (AAV), recombinant adenoviruses, recombinant lentiviruses, recombinant retroviruses, recombinant herpes simplex viruses, recombinant poxviruses, phages, etc. The present disclosure provides vectors capable of integration in the host genome, such as retrovirus or lentivirus. See, e.g., Ausubel et al., Current Protocols in Molecular Biology, John Wiley & Sons, New York, 1989; Kay, M. A., et al., 2001 Nat. Medic. 7(1):33-40; and Walther W. and Stein U., 2000 Drugs, 60(2): 249-71, incorporated herein by reference.

In one embodiment, a DNA segment encoding an engineered nuclease, fusion protein, or one or more or all of the components of the system or compositions is contained in a plasmid vector that allows expression of the protein(s) and subsequent isolation and purification produced by the recombinant vector. Accordingly, the proteins disclosed herein can be purified following expression, obtained by chemical synthesis, or obtained by recombinant methods.

To construct cells that express an engineered nuclease, fusion protein, or one or more or all of the components of the system or compositions, expression vectors for stable or transient expression may be constructed via conventional methods as described herein and introduced into cells. For example, nucleic acids encoding an engineered nuclease, fusion protein, or one or more or all of the components of the system or compositions may be cloned into a suitable expression vector, such as a plasmid or a viral vector in operable linkage to a suitable promoter. The selection of expression vectors/plasmids/viral vectors should be suitable for integration and replication in eukaryotic cells.

In certain embodiments, vectors of the present disclosure can drive the expression of one or more sequences in prokaryotic cells. Promoters that may be used include T7 RNA polymerase promoters, constitutive E. coli promoters, and promoters that could be broadly recognized by transcriptional machinery in a wide range of bacterial organisms. The system may be used with various bacterial hosts.

In certain embodiments, vectors of the present disclosure can drive the expression of one or more sequences in mammalian cells using a mammalian expression vector. Examples of mammalian expression vectors include pCDM8 (Seed, Nature (1987) 329:840, incorporated herein by reference) and pMT2PC (Kaufman, et al., EMBO J. (1987) 6:187, incorporated herein by reference). When used in mammalian cells, the expression vector's control functions are typically provided by one or more regulatory elements. For example, commonly used promoters are derived from polyoma, adenovirus 2, cytomegalovirus, simian virus 40, and others disclosed herein and known in the art. For other suitable expression systems for both prokaryotic and eukaryotic cells see, e.g., Chapters 16 and 17 of Sambrook, et al., MOLECULAR CLONING: A LABORATORY MANUAL. 2nd eds., Cold Spring Harbor Laboratory, Cold Spring Harbor Laboratory Press, Cold Spring Harbor, N.Y., 1989, incorporated herein by reference.

Vectors of the present disclosure can comprise any of a number of promoters known to the art, wherein the promoter is constitutive, regulatable or inducible, cell type specific, tissue-specific, or species specific. In addition to the sequence sufficient to direct transcription, a promoter sequence of the invention can also include sequences of other regulatory elements that are involved in modulating transcription (e.g., enhancers, Kozak sequences and introns). Many promoter/regulatory sequences useful for driving constitutive expression of a gene are available in the art and include, but are not limited to, for example, CMV (cytomegalovirus promoter), EF1a (human elongation factor 1 alpha promoter), SV40 (simian vacuolating virus 40 promoter), PGK (mammalian phosphoglycerate kinase promoter), Ubc (human ubiquitin C promoter), human beta-actin promoter, rodent beta-actin promoter, CBh (chicken beta-actin promoter), CAG (hybrid promoter contains CMV enhancer, chicken beta actin promoter, and rabbit beta-globin splice acceptor), TRE (Tetracycline response element promoter), H1 (human polymerase III RNA promoter), U6 (human U6 small nuclear promoter), and the like. Additional promoters that can be used for expression of the components of the present system, include, without limitation, cytomegalovirus (CMV) intermediate early promoter, a viral LTR such as the Rous sarcoma virus LTR, HIV-LTR, HTLV-1 LTR, Maloney murine leukemia virus (MMLV) LTR, myeoloproliferative sarcoma virus (MPSV) LTR, spleen focus-forming virus (SFFV) LTR, the simian virus 40 (SV40) early promoter, herpes simplex tk virus promoter, elongation factor 1-alpha (EF1-α) promoter with or without the EF1-α intron. Additional promoters include any constitutively active promoter. Alternatively, any regulatable promoter may be used, such that its expression can be modulated within a cell.

Moreover, inducible and tissue specific expression can be accomplished by placing the nucleic acid encoding such a molecule under the control of an inducible or tissue specific promoter/regulatory sequence. Examples of tissue specific or inducible promoter/regulatory sequences which are useful for this purpose include, but are not limited to, the rhodopsin promoter, the MMTV LTR inducible promoter, the SV40 late enhancer/promoter, synapsin 1 promoter, ET hepatocyte promoter, GS glutamine synthase promoter and many others. In addition, promoters which are well known in the art can be induced in response to inducing agents such as metals, glucocorticoids, tetracycline, hormones, and the like, are also contemplated for use with the invention. Thus, it will be appreciated that the present disclosure includes the use of any promoter/regulatory sequence capable of driving expression of the desired protein operably linked thereto.

The vectors of the present disclosure may direct expression of the nucleic acid in a particular cell type (e.g., tissue-specific regulatory elements are used to express the nucleic acid). Such regulatory elements include promoters that may be tissue specific or cell specific. The term “tissue specific” as it applies to a promoter refers to a promoter that is capable of directing selective expression of a nucleotide sequence of interest to a specific type of tissue (e.g., seeds) in the relative absence of expression of the same nucleotide sequence of interest in a different type of tissue. The term “cell type specific” as applied to a promoter refers to a promoter that is capable of directing selective expression of a nucleotide sequence of interest in a specific type of cell in the relative absence of expression of the same nucleotide sequence of interest in a different type of cell within the same tissue. The term “cell type specific” when applied to a promoter also means a promoter capable of promoting selective expression of a nucleotide sequence of interest in a region within a single tissue. Cell type specificity of a promoter may be assessed using methods well known in the art, e.g., immunohistochemical staining.

Additionally, the vector may contain, for example, some or all of the following: a selectable marker gene, such as the neomycin gene for selection of stable or transient transfectants in host cells; enhancer/promoter sequences from the immediate early gene of human CMV for high levels of transcription; transcription termination and RNA processing signals from SV40 for mRNA stability; 5′- and 3′-untranslated regions for mRNA stability and translation efficiency from highly-expressed genes like α-globin or β-globin; SV40 polyoma origins of replication and ColE1 for proper episomal replication; internal ribosome binding sites (IRESes), versatile multiple cloning sites; T7 and SP6 RNA promoters for in vitro transcription of sense and antisense RNA; a “suicide switch” or “suicide gene” which when triggered causes cells carrying the vector to die (e.g., HSV thymidine kinase, an inducible caspase such as iCasp9), and reporter gene for assessing expression of the chimeric receptor. Suitable vectors and methods for producing vectors containing transgenes are well known and available in the art. Selectable markers also include chloramphenicol resistance, tetracycline resistance, spectinomycin resistance, streptomycin resistance, erythromycin resistance, rifampicin resistance, bleomycin resistance, thermally adapted kanamycin resistance, gentamycin resistance, hygromycin resistance, trimethoprim resistance, dihydrofolate reductase (DHFR), GPT; the URA3, HIS4, LEU2, and TRP1 genes of S. cerevisiae.

When introduced into the cell, the vectors may be maintained as an autonomously replicating sequence or extrachromosomal element or may be integrated into host DNA.

The proteins (e.g., engineered nucleases, fusion protein, effector domains), polynucleotides encoding these proteins, and systems and compositions comprising the proteins and/or polynucleotides described herein may be delivered by any suitable means. In certain embodiments, the delivery is in vivo. In other embodiments, the delivery is to isolated/cultured cells (e.g., autologous iPS cells) in vitro to provide modified cells useful for in vivo delivery to patients afflicted with a disease or condition.

Vectors according to the present disclosure can be transformed, transfected, or otherwise introduced into a wide variety of cells. Transfection refers to the taking up of a vector by a cell whether or not any coding sequences are in fact expressed. Numerous methods of transfection are known to the ordinarily skilled artisan, for example, lipofectamine, calcium phosphate co-precipitation, electroporation, DEAE-dextran treatment, microinjection, viral infection, and other methods known in the art. Transduction refers to entry of a virus into the cell and expression (e.g., transcription and/or translation) of sequences delivered by the viral vector genome. In the case of a recombinant vector, “transduction” generally refers to entry of the recombinant viral vector into the cell and expression of a nucleic acid of interest delivered by the vector genome.

Any of the vectors comprising a nucleic acid sequence that encodes an engineered nuclease, fusion protein, or one or more or all of the components of the system or compositions is also within the scope of the present disclosure. Such a vector may be delivered into host cells by a suitable method. Methods of delivering vectors to cells are well known in the art and may include DNA or RNA electroporation, transfection reagents such as liposomes or nanoparticles to delivery DNA or RNA; delivery of DNA, RNA, or protein by mechanical deformation (see, e.g., Sharei et al. Proc. Natl. Acad. Sci. USA 110(6): 2082-2087(2013) incorporated herein by reference); or viral transduction. In some embodiments, the vectors are delivered to host cells by viral transduction. Nucleic acids can be delivered as part of a larger construct, such as a plasmid or viral vector, or directly, e.g., by electroporation, lipid vesicles, viral transporters, microinjection, and biolistics (high-speed particle bombardment). Similarly, the construct containing the one or more transgenes can be delivered by any method appropriate for introducing nucleic acids into a cell. In some embodiments, the construct or the nucleic acid encoding the components of the present system is a DNA molecule. In some embodiments, the nucleic acid encoding the components of the present system is a DNA vector and may be electroporated to cells. In some embodiments, the nucleic acid encoding the components of the present system is an RNA molecule, which may be electroporated to cells.

Additionally, delivery vehicles such as nanoparticle- and lipid-based mRNA or protein delivery systems can be used. Further examples of delivery vehicles include lentiviral vectors, ribonucleoprotein (RNP) complexes, lipid-based delivery system, gene gun, hydrodynamic, electroporation or nucleofection microinjection, and biolistics. Various gene delivery methods are discussed in detail by Nayerossadat et al. (Adv Biomed Res. 2012; 1:27) and Ibraheem et al. (Int J Pharm. 2014 Jan. 1; 459(1-2):70-83), incorporated herein by reference.

In some embodiments, the engineered nucleases, fusion proteins, or one or more or all of the components of the system or compositions may be mixed, individually or in any combination, with a carrier which are also within the scope of the present disclosure. Exemplary carriers include buffers, antioxidants, preservatives, carbohydrates, surfactants, and the like, and are described in detail elsewhere herein.

Also disclosed are cells comprising the engineered nucleases, fusion proteins, nucleic acids, or one or more or all of the components of the system or compositions described herein. In some embodiments, the cell is a prokaryotic cell. In some embodiments, the cell is a eukaryotic cell. In some embodiments, the cell is a mammalian cell. In some embodiments, the cell is a human cell.

### Methods

The disclosure also provides methods of modifying a target nucleic acid sequence. The phrase “modifying a nucleic acid sequence,” as used herein, refers to modifying at least one physical feature of a nucleic acid sequence of interest or a functional feature of a nucleic acid sequence. In some embodiments, the nucleic acid alterations include, for example, single or double strand breaks, deletion, or insertion of one or more nucleotides, and other modifications that affect the structural integrity or nucleotide sequence of the nucleic acid sequence.

In some embodiments, the methods introduce a single strand or double strand break in the target nucleic acid sequence. In this respect, the disclosed systems may direct cleavage of one or two strands of a target nucleic acid sequence, such as within a target genomic DNA sequence and/or within the complement of the target sequence.

In some embodiments, altering a nucleic acid sequence comprises a deletion. The deletion may be upstream or downstream of nuclease binding site, so called unidirectional deletions. The deletion may encompass sequences on either side of the binding site, a bidirectional deletion. The deletion of the nucleic sequence may be of any size. The methods can be used to delete nucleic acids from a target sequence in a host cell by cleaving the target sequence and allowing the host cell to repair the cleaved sequence. Deletion of a nucleic acid sequence in this manner can be used in a variety of applications, such as, for example, to remove disease-causing trinucleotide repeat sequences in neurons, to create gene knock-outs or knock-downs, and to generate mutations for disease models in research.

In some embodiments, the systems and methods described herein may be used to insert a gene or fragment thereof into a cell. For example, the systems or methods may include an exogenous nucleic acid molecule which encodes a gene protein (e.g., a nucleic acid encoding a gene or gene product) which is inserted at the site of nucleic acid cleavage.

In some embodiments, the methods do not introduce a single strand or double strand break in the target nucleic acid sequence. The methods may result in modifying the nucleic acid sequence as a result of one of more of the effector or functional domains as described above. For example, the methods may modulate the transcription of a target nucleic acid, may add or remove moieties from the target nucleic acid (e.g., methyl groups), may edit bases in the target nucleic acid (e.g., deaminate, depurinate, depyrimidinate), may unwind, replication, of combine target nucleic acids, and/or may add or remove moieties from histones (e.g., methylate, demethylate, acetylate, deacetylate, ubiquitinate, phosphorylate, sumoylate) bound to the nucleic acid.

The methods comprise contacting a target nucleic acid sequence with an engineered nuclease, fusion protein, composition, or system as described herein. In some embodiments, contacting a target nucleic acid sequence comprises introducing the engineered nuclease, fusion protein, composition, or system into the cell. The engineered nuclease, fusion protein, composition, or system may be introduced into eukaryotic or prokaryotic cells by methods known in the art, as described elsewhere herein.

The cell may be a prokaryotic cell, a plant cell, an insect cell, a vertebrate cell, an invertebrate cell, an animal cell, a mammalian cell, or a human cell. In some embodiments, the cell is a stem cell.

In some embodiments, the cell is ex vivo (e.g., fresh isolate-early passage). In some cases, the cell is in vivo. In some cases, the cell is in culture or in vitro (e.g., immortalized cell line). Cells may be from established cell lines or they may be primary cells, where “primary cells,” “primary cell lines,” and “primary cultures” are used interchangeably herein to refer to cells and cells cultures that have been derived from a subject and allowed to grow in vitro for a limited number of passages of the culture. For example, primary cultures are cultures that may have been passaged 0 times, 1 time, 2 times, 4 times, 5 times, 10 times, or 15 times, but not enough times go through the crisis stage. Typically, the primary cell lines are maintained for fewer than 10 passages in culture.

In some embodiments, introducing the engineered nuclease, fusion protein, composition, or system into a cell comprises administering the engineered nuclease, fusion protein, composition, or system to a subject. In some embodiments, the subject is human. The administering may comprise in vivo administration of the engineered nuclease, fusion protein, composition, system, or a nucleic acid encoding the engineered nuclease, fusion protein, or system. In alternative embodiments, an in vitro or ex vivo treated cell is transplanted into a subject.

In some embodiments, the target nucleic acid is a nucleic acid endogenous to a target cell. In some embodiments, the target nucleic acid is a genomic DNA sequence. The term “genomic,” as used herein, refers to a nucleic acid sequence (e.g., a gene or locus) that is located on a chromosome in a cell.

In some embodiments, the target nucleic acid encodes a gene or gene product. The term “gene product,” as used herein, refers to any biochemical product resulting from expression of a gene. Gene products may be RNA or protein. RNA gene products include non-coding RNA, such as tRNA, rRNA, micro RNA (miRNA), and small interfering RNA (siRNA), and coding RNA, such as messenger RNA (mRNA). In some embodiments, the target nucleic acid sequence encodes a protein or polypeptide.

The present methods may be used in various bacterial hosts, including human pathogens that are medically important, and bacterial pests that are key targets within the agricultural industry, as well as antibiotic resistant versions thereof. The method may be designed to target any gene or any set of genes, such as virulence or metabolic genes, for clinical and industrial applications in other embodiments. The present methods may be used to inactivate microbial genes. In some embodiments, the gene is an antibiotic resistance gene. The present methods may be used to treat a multi-drug resistance bacterial infection in a subject. The present methods may also be used for genomic engineering within complex bacterial consortia.

The methods described here also provide for treating a disease or disorder in a subject. The method may comprise administering to the subject, in vivo, an effective amount of the engineered nuclease, fusion protein, composition, or system, or by transplantation of ex vivo treated cells. A “subject” or “patient” may be human or non-human and may include, for example, animal strains or species used as “model systems” for research purposes, such a mouse model as described herein. Within the context of the present disclosure, the term “effective amount” refers to that quantity such that modification of the target nucleic acid is achieved.

In some embodiments, the systems and methods target one or more “disease-associated” genes. The term “disease-associated gene,” refers to any gene or polynucleotide whose gene products are expressed at an abnormal level or in an abnormal form in cells obtained from a disease-affected individual as compared with tissues or cells obtained from an individual not affected by the disease. A disease-associated gene may refer to a gene, the mutation or genetic variation of which is directly responsible or is in linkage disequilibrium with a gene(s) that is responsible for the etiology of a disease. In another embodiment, the target genomic DNA sequence can comprise a gene, the mutation of which contributes to a particular disease in combination with mutations in other genes.

When utilized as a method of treatment, the effective amount may depend on the particular condition being treated, the severity of the condition, the individual patient parameters including age, physical condition, size, gender and weight, the duration of the treatment, the nature of concurrent therapy (if any), the specific route of administration and like factors within the knowledge and expertise of the health practitioner. In some embodiments, the effective amount alleviates, relieves, ameliorates, improves, reduces the symptoms, or delays the progression of any disease or disorder in the subject. In some embodiments, the subject is a human.

A wide range of additional therapies may be used in conjunction with the methods of the present disclosure. The additional therapy may be administration of a therapeutic agent or may be an additional therapy not connected to administration of a therapeutic agent. Such additional therapies include, but are not limited to, surgery, immunotherapy, radiotherapy. The additional therapy may be administered at the same time as the above methods. In some embodiments, the additional therapy may precede or follow the treatment of the disclosed methods by time intervals ranging from hours to months.

In some embodiments, effective combination therapy is achieved with a single composition or pharmacological formulation or with two distinct compositions or formulations, administered at the same time or separated by a time interval. The therapeutic agent may comprise any manner of therapeutic, including protein, small molecule, nucleic acids, and the like. For example, therapeutic agents include, but are not limited to, immune modulators, chemotherapeutic agents, a nucleic acid (e.g., mRNA, aptamers, antisense oligonucleotides, ribozyme nucleic acids, interfering RNAs, antigene nucleic acids), decongestants, steroids, analgesics, antimicrobial agents, immunotherapies, or any combination thereof.

In the context of the present disclosure insofar as it relates to any of the disease conditions recited herein, the terms “treat,” “treatment,” and the like mean to relieve or alleviate at least one symptom associated with such condition, or to slow or reverse the progression of such condition. Within the meaning of the present disclosure, the term “treat” also denotes to arrest, delay the onset (e.g., the period prior to clinical manifestation of a disease) and/or reduce the risk of developing or worsening a disease. For example, in connection with cancer the term “treat” may mean elimination or reduction of a patient's tumor burden, or prevention, delay, or inhibition of metastasis, etc.

The methods disclosed herein are also applicable to plants. For example, the methods can be used to generate novel engineered plants to improve agronomic traits, for example, herbicidal resistance, resistance to environmental stress, resistance to pests, etc.

The disclosed engineered nucleases, fusion proteins, compositions, and systems can be introduced into a plant, or a plant cell, seed, fruit, plant part, or propagation material of the plant. The term “plant propagation material” refers to generative parts of a plant, which can be used for the multiplication of the plant, and vegetative plant material such as cuttings and tubers (e.g., potatoes). In some embodiments, the propagation material is a root, a corm, a tuber, a bulb, a slip, a cutting of the plant, and a rhizome. Parts of a plant are any sections of a plant (e.g., roots, cotyledons, tendrils, leaves, flowers, seeds, stems, callus tissue, nuts, and fruit) that develop from a plant propagation material or grow at a later time. The methods described herein can be used on any plant part. Examples of plant parts include but are not limited to the root, corm, tuber, bulb, slip and rhizome.

Methods of introducing exogenous nucleic acids into plant cells are well known in the art. Such plant cells are considered transformed. DNA constructs can be introduced into plant cells by various methods, including, but not limited to PEG- or electroporation-mediated protoplast transformation, tissue culture or plant tissue transformation by biolistic bombardment, or the Agrobacterium-mediated transient and stable transformation.

The transformation can be a transient or a stable transformation. As used herein, the term “stable transformation” is intended to mean that the nucleotide construct introduced into a plant integrates into the genome of the plant and is capable of being inherited by the progeny thereof. “Transient transformation” is intended to mean that a polynucleotide is introduced into the plant and does not integrate into the genome of the plant or a polypeptide is introduced into a plant. In select embodiments, the nucleic acid encoding the RNA hairpin may be stably integrated into the plant genome, for example via Agrobacterium-mediated transformation.

Suitable methods also include viral infection (such as double stranded DNA viruses), transfection, conjugation, protoplast fusion, electroporation, particle gun technology, calcium phosphate precipitation, direct microinjection, silicon carbide whiskers technology, Agrobacterium-mediated transformation, and the like. The choice of method is generally dependent on the type of cell being transformed and the circumstances under which the transformation is taking place (e.g., in vitro, ex vivo, or in vivo). Transformation methods based upon the soil bacterium Agrobacterium tumefaciens are useful for introducing an exogenous nucleic acid molecule into a vascular plant. The wild-type form of Agrobacterium contains a Ti (tumor-inducing) plasmid that directs production of tumorigenic crown gall growth on host plants. Transfer of the tumor-inducing T-DNA region of the Ti plasmid to a plant genome requires the Ti plasmid-encoded virulence genes as well as T-DNA borders, which are a set of direct DNA repeats that delineate the region to be transferred. An Agrobacterium-based vector is a modified form of a Ti plasmid, in which the tumor inducing functions are replaced by the nucleic acid sequence of interest to be introduced into the plant host.

Agrobacterium-mediated transformation generally employs cointegrate vectors or binary vector systems, in which the components of the Ti plasmid are divided between a helper vector, which resides permanently in the Agrobacterium host and carries the virulence genes, and a shuttle vector, which contains the gene of interest bounded by T-DNA sequences. A variety of binary vectors are well known in the art and are commercially available, for example, from Clontech (Palo Alto, Calif.). Methods of co-culturing Agrobacterium with cultured plant cells or wounded tissue such as leaf tissue, root explants, hypocotyledons, stem pieces or tubers, for example, also are well known in the art. See., e.g., Glick and Thompson, (eds.), Methods in Plant Molecular Biology and Biotechnology, Boca Raton, Fla.: CRC Press (1993), incorporated herein by reference.

Microprojectile-mediated transformation also can be used. This method, first described by Klein et al. (Nature 327:70-73 (1987), incorporated herein by reference), relies on microprojectiles such as gold or tungsten that are coated with the desired nucleic acid molecule by precipitation with calcium chloride, spermidine, or polyethylene glycol. The microprojectile particles are accelerated at high speed into an angiosperm tissue using a device such as the BIOLISTIC PD-1000 (Biorad; Hercules Calif.).

As such, the disclosure also provides plants and plant propagation materials (e.g., plant cell, seed, fruit, or plant parts) produced using the methods disclosed herein. Genetically modified, transformed, or transgenic plants include plants into which an exogenous polynucleotide, e.g., a polynucleotide encoding the engineered nuclease or fusion protein disclosed herein, has been introduced.

The methods disclosed herein are suitable for use with any plant, for example, grain crops, fruit crops, forage crops, root vegetable crops, leafy vegetable crops, flowering plants, conifers, trees, oil crops, plants used in phytoremediation, industrial crops, medicinal crops, laboratory model plants, and the like. As such, non-limiting examples of plants that may be used with the present methods include: grains, forage crops, fruits, vegetables, oil seed crops, palms, forestry, vines, maize (corn, Zea mays), banana, peanut, field peas, sunflower, tomato, canola, tobacco, wheat, barley, oats, potato, soybeans, cotton, carnations, sorghum, lupin, rice, rutabaga, celery, switchgrass, apple, petunias, Arabidopsis thaliana, Medicago truncatula, Medicago sativa, Brachypodium distachyon, Nicotiana benthamiana, or Setaria viridis.

### Kits

Also within the scope of the present disclosure are kits that include the engineered nucleases, fusion proteins, nucleic acids, cells, compositions, systems, or components thereof as disclosed herein.

The kits may contain one or more reagents or other components useful, necessary, or sufficient for practicing any of the methods described herein, such as, transfection or administration reagents, negative and positive control samples (e.g., cells, template DNA), cells, containers housing one or more components (e.g., microcentrifuge tubes, boxes), detectable labels, detection and analysis instruments, software, instructions, and the like.

The kit may include instructions for use in any of the methods described herein. Kits optionally may provide additional components such as buffers and interpretive information. Normally, the kit comprises a container and a label or package insert(s) on or associated with the container. Instructions supplied in the kits of the disclosure are typically written instructions on a label or package insert. In some embodiment, the disclosure provides articles of manufacture comprising contents of the kits described above.

The kits provided herein are in suitable packaging. Suitable packaging includes, but is not limited to, vials, bottles, jars, flexible packaging, and the like. A kit may have a sterile access port (for example, the container may be an intravenous solution bag or a vial having a stopper pierceable by a hypodermic injection needle). The container may also have a sterile access port.

The packaging may be unit doses, bulk packages (e.g., multi-dose packages) or sub-unit doses.

The kit will typically be provided with its various components in one or more packages, e.g., a fiber-based, a cardboard, polymeric, or a Styrofoam box. The enclosure(s) can be configured so as to maintain a temperature differential between the interior and the exterior, for example, to provide insulating properties to keep the reagents at a preselected temperature for a preselected time. The packaging can be air-tight, waterproof (e.g., impermeable to changes in moisture or evaporation), and/or light-tight.

### Examples

The following are examples of the present invention and are not to be construed as limiting.

### Materials and Methods

### Cell Culture, Plasmid Construction, and Transient Transfection

HEK293T cells (ATCC) were cultured at 37° C. and 5% (v/v) CO₂ in high glucose DMEM with 4 mM L-glutamine, 1 mM sodium pyruvate and phenol red pH indicator (Gibco), supplemented with 10% FBS and 1X penicillin-streptomycin. 24 hours prior to transfection, cells were seeded at a density of 10³ cells/well in 96 well tissue culture-treated plates.

The guide RNA (gRNA) was cloned into pGuide plasmid with a U6 promoter driving its expression and a CMV-driven GFP transfection reporter using HiFi DNA Assembly protocols (New England Biolabs). Nuclease enzyme constructs were assembled using HiFi DNA Assembly (New England Biolabs) of Gene Fragments (Twist Biosciences) into the pTwist CMV vector.

For each transfection well, 50 ng of gRNA plasmid and 50 ng of nuclease enzyme plasmid or base editor enzyme plasmid were added to 5 μL of Opti-MEM (Gibco). One non-targeting guide RNA negative control was included for each experiment. 0.2 μL of TransIT®-2020 transfection reagent (Mirus Bio) was diluted into 4 μL of Opti-MEM. Plasmid and TransIT®-2020 mixtures were combined, incubated for 15-30 min at room temperature, and added to HEK293T cells in a dropwise manner. Plates were gently rocked to mix and incubated for 72 hours. Guide RNA sequences are listed in Table 1.

### Microscopy, Sample Preparation, and Sequencing

72 hours post-transfection, qualitative assessments of transfection efficiency and cell health were made using fluorescence and brightfield microscopy (Revolve Echo). Quantitative transfection efficiency measurements were made using 96 well plate-based fluorescence measurements (Tecan Spark). Culture media was aspirated from cells prior to washing with PBS (Gibco). 35 μL lysis buffer (100 mM Tris-HCl, pH 7.5; 0.05% SDS; 25 μg/mL Proteinase K) was added to each well. The samples were then incubated at 37° C. for 1 hour before being transferred to 96-well PCR plates and boiled at 98° C. for 15 min. Guide-specific primers were used to PCR amplify the genomic target region of interest from the cell lysates (Q5 DNA Polymerase NEB). Five samples per plate were spot-checked via gel electrophoresis to confirm the presence of the expected amplicon size. The Mag-Bind® RxnPure Plus (Omega Bio-tek) PCR clean-up kit was used to purify the PCR products prior to sequencing submission. Once eluted, DNA yields were quantified via the QuantiFluor® kit (Promega). DNA concentrations were normalized to 2 ng/100 bp of amplicon length and submitted to the UC Berkeley Sequencing facility for Sanger sequencing along with the appropriate forward PCR primer. PCR and Sanger sequencing primers are listed in Table 2.

### Sequencing Analysis

To determine the editing efficiency of nucleases from Sanger sequencing data, the open source Inference of CRISPR Edits program (ICE) v1.2.0 was used, using default parameters (Conant et al. 2022; liebertpub.com/doi/full/10.1089/crispr.2021.0113; github.com/synthego-open/ice). ICE determines potential outcomes for editing with guide RNAs which are supported by the Sanger data via regression. The ICE algorithm produces accurate estimates of editing compared to other Sanger analysis tools, and compared to NGS-based quantification of editing.

For indel quantification, target sites were amplified using a two-step PCR reaction. First, 5 μL of lysate (corresponding to ˜ 10,000 cells) was used as a template for PCR with (Platinum™ SuperFi II PCR Master Mix, Thermo Fisher Scientific) unique primer pairs containing an internal locus-specific region and an outer Illumina-compatible adapter sequence (Table 3). The resulting product was then diluted 1:100 in molecular biology grade water and used as a template in a second PCR reaction (Q5® High-Fidelity DNA Polymerase, New England Biolabs) targeting the outer-adapter sequence, appending unique indices to each amplicon for pooled sequencing (xGen™ UDI 10nt Primer Plates 1-16, Catalog number: 10008054, Integrated DNA Technologies). Amplicons were pooled 1:1 and sequenced on a NovaseqX with 2×151 paired end reads (Seqmatic). All amplicons across experiments included reference samples that were not treated with active nuclease to control for any variant reads relative to reference genome that are not due to gene editing. After de-multiplexing, ˜0.025M-6M reads per amplicon were obtained. Reads were trimmed and filtered using fastp removing ˜2-5% of reads per sample. Next, CRISPResso2 v2.2.24 was run in “CRISPRessoBatch” mode with default parameters using the processed reads, amplicon sequences, and spacer sequences as input. For off-target sites, spacer sequences were edited to match the corresponding region on the amplicon. The indel rate was computed as the percentage of aligned reads having an insertion or deletion within one bp of the cleavage site. Eight amplicons which displayed low read quality, low alignment rates, high rates of substitutions, or high rates of editing at negative controls were excluded.

To quantify base editing efficiency from Sanger sequencing data, an open source program called Beat was used using default parameters (Xu et al. CRISPR J 2019). Beat is a Python-based program that determines editing efficiency after subtracting the background noise and without the need to normalize to control samples. Beat produces results that are concordant with EditR (another commonly used software for base editing) and agree with base editing quantification based on NGS.

TABLE 1

| | | SEQ |
|---|---|---|
| Guide RNA ID | Guide sequence | ID NO |
| HEK3-gRNA | GGCCCAGACUGAGCACGUGA | 400 |
| CD3G_1-gRNA | ACAUACUUCUGUAAUACACU | 401 |
| HEK2-gRNA | GAACACAAAGCAUAGACUGC | 402 |
| Non-targeting-gRNA | GAGCAGAUCGUUGAUUGUAG | 403 |
| control | | |
| AAVS1-on | GGGGCCACUAGGGACAGGAU | 413 |
| AAVS1-off1 | GGGGCCACUAGGGACAGGAU | 414 |
| AAVS1-off2 | GGGGCCACUAGGGACAGGAU | 415 |
| AAVS1-off3 | GGGGCCACUAGGGACAGGAU | 416 |
| FANCF-on | GGAAUCCCUUCUGCAGCACC | 417 |
| FANCF-off1 | GGAAUCCCUUCUGCAGCACC | 418 |
| FANCF-off2 | GGAAUCCCUUCUGCAGCACC | 419 |
| FANCF-off3 | GGAAUCCCUUCUGCAGCACC | 420 |
| VEGFA-on | GGGUGGGGGGAGUUUGCUCC | 421 |
| VEGFA-off1 | GGGUGGGGGGAGUUUGCUCC | 422 |
| VEGFA-off2 | GGGUGGGGGGAGUUUGCUCC | 423 |
| VEGFA-off3 | GGGUGGGGGGAGUUUGCUCC | 424 |
| HEK2-on | GAACACAAAGCAUAGACUGC | 425 |
| HEK2-off1 | GAACACAAAGCAUAGACUGC | 426 |
| HEK2-off2 | GAACACAAAGCAUAGACUGC | 427 |
| HEK3-on | GGCCCAGACUGAGCACGUGA | 428 |
| HEK3-off1 | GGCCCAGACUGAGCACGUGA | 429 |
| HEK3-off2 | GGCCCAGACUGAGCACGUGA | 430 |
| HEK3-off3 | GGCCCAGACUGAGCACGUGA | 431 |
| T39 | GGACAGCUUUUCCUAGACAG | 432 |

TABLE 2

| PCR and Sanger | | SEQ |
|---|---|---|
| Sequencing | | ID |
| Primer | Sequence | NO: |
| HEK3 forward | ATGTGGGCTGCCTAGAAAGG | 404 |
| HEK3 reverse | CCCAGCCAAACTTGTCAACC | 405 |
| CD3G_1 forward | GACGCAGATATTGGCGTCAC | 406 |
| CD3G_1 reverse | GGAAGCCCATGTAGTCTAACTACC | 407 |
| HEK2 forward | CTGGTGGTACTTGAATCAAGCAC | 408 |
| HEK2 reverse | GAAGGAGACTTGTGCACATTCTATAG | 409 |
| T39 forward | GCCTGTGATGGGCTAATTGATG | 433 |
| T39 reverse | GTCTCTGACCACTGCGATATG | 434 |

TABLE 3

| Illumina | | |
|---|---|---|
| NGS | | SEQ |
| Sequencing | | ID |
| Primer | Sequence | NO: |
| AAVS1-on_fwd | CACTCTTTCCCTACACGACGCTCTTCCGA | 435 |
| | TCTACAGGAGGTGGGGGTTAGAC | |
| AAVS1-on_rev | GGAGTTCAGACGTGTGCTCTTCCGATCTC | 436 |
| | GGTTAATGTGGCTCTGGTT | |
| AAVS1-off1_fwd | CACTCTTTCCCTACACGACGCTCTTCCGA | 437 |
| | TCTAACACCACCACTGCTTACCC | |
| AAVS1-off1_rev | GGAGTTCAGACGTGTGCTCTTCCGATCTT | 438 |
| | CCATTAAGTTCCAGGGGTG | |
| AAVS1-off2_fwd | CACTCTTTCCCTACACGACGCTCTTCCGA | 439 |
| | TCTAGAGGTCACCCTCACCACAC | |
| AAVS1-off2_rev | GGAGTTCAGACGTGTGCTCTTCCGATCTT | 440 |
| | GTTCACAGCTTGTAGGCCA | |
| AAVS1-off3_fwd | CACTCTTTCCCTACACGACGCTCTTCCGA | 441 |
| | TCTCTTGGCTCCTTCGTTACAGC | |
| AAVS1-off3_rev | GGAGTTCAGACGTGTGCTCTTCCGATCTG | 442 |
| | GTAGATGGGCAACCAACTC | |
| FANCF-on_fwd | CACTCTTTCCCTACACGACGCTCTTCCGA | 443 |
| | TCTCTTGCCTCCACTGGTTGTG | |
| FANCF-on_rev | GGAGTTCAGACGTGTGCTCTTCCGATCTG | 444 |
| | CCGTCTCCAAGGTGAAA | |
| FANCF-off1_fwd | CACTCTTTCCCTACACGACGCTCTTCCGA | 445 |
| | TCTGCCTGCTGCACTCTCTGAGTA | |
| FANCF-off1_rev | GGAGTTCAGACGTGTGCTCTTCCGATCTC | 446 |
| | ACAGATTGATGCCACTGGA | |
| FANCF-off2_fwd | CACTCTTTCCCTACACGACGCTCTTCCGA | 447 |
| | TCTCGAACTCCTGGGGCTTCT | |
| FANCF-off2_rev | GGAGTTCAGACGTGTGCTCTTCCGATCTG | 448 |
| | TGGGGAGGGAGAGCAGT | |
| FANCF-off3_fwd | CACTCTTTCCCTACACGACGCTCTTCCGA | 449 |
| | TCTGTGACCCAGGTCCAGTGTTT | |
| FANCF-off3_rev | GGAGTTCAGACGTGTGCTCTTCCGATCTT | 450 |
| | GAGACCTCTGACCTCCACA | |
| VEGFA-on_fwd | CACTCTTTCCCTACACGACGCTCTTCCGA | 451 |
| | TCTCCAGATGGCACATTGTCAGA | |
| VEGFA-on_rev | GGAGTTCAGACGTGTGCTCTTCCGATCTG | 452 |
| | CTAAAGAGGGAATGGGCTT | |
| VEGFA-off1_fwd | CACTCTTTCCCTACACGACGCTCTTCCGAT | 453 |
| | CTCATCAGGCAATGAGTCAGGA | |
| VEGFA-off1_rev | GGAGTTCAGACGTGTGCTCTTCCGATCTTC | 454 |
| | TTAGGAGGTTGGGTGTGC | |
| VEGFA-off2_fwd | CACTCTTTCCCTACACGACGCTCTTCCGAT | 455 |
| | CTTCTATCCTTTCCAAGGGCAA | |
| VEGFA-off2_rev | GGAGTTCAGACGTGTGCTCTTCCGATCTTC | 456 |
| | AATAACAAGGCTCCCAGG | |
| VEGFA-off3_fwd | CACTCTTTCCCTACACGACGCTCTTCCGAT | 457 |
| | CTGTGAAGCGTGACACTCCCTT | |
| VEGFA-off3_rev | GGAGTTCAGACGTGTGCTCTTCCGATCTTG | 458 |
| | CTCAGTCCAGAGCTCTTCT | |
| HEK2-on_fwd | CACTCTTTCCCTACACGACGCTCTTCCGAT | 459 |
| | CTCCAGCCCCATCTGTCAAACT | |
| HEK2-on_rev | GGAGTTCAGACGTGTGCTCTTCCGATCTTG | 460 |
| | AATGGATTCCTTGGAAACAATGA | |
| HEK2-off1_fwd | CACTCTTTCCCTACACGACGCTCTTCCGAT | 461 |
| | CTGTGTGGAGAGTGAGTAAGCCA | |
| HEK2-off1_rev | GGAGTTCAGACGTGTGCTCTTCCGATCTAC | 462 |
| | GGTAGGATGATTTCAGGCA | |
| HEK2-off2_fwd | CACTCTTTCCCTACACGACGCTCTTCCGAT | 463 |
| | CTTTTTTGGTACTCGAGTGTTATTCAG | |
| HEK2-off2_rev | GGAGTTCAGACGTGTGCTCTTCCGATCTCA | 464 |
| | CAAAGCAGTGTAGCTCAGG | |
| HEK3-on_fwd | CACTCTTTCCCTACACGACGCTCTTCCGAT | 465 |
| | CTATGTGGGCTGCCTAGAAAGG | |
| HEK3-on_rev | GGAGTTCAGACGTGTGCTCTTCCGATCTCC | 466 |
| | CAGCCAAACTTGTCAACC | |
| HEK3-off1_fwd | CACTCTTTCCCTACACGACGCTCTTCCGAT | 467 |
| | CTTCCCCTGTTGACCTGGAGAA | |
| HEK3-off1_rev | GGAGTTCAGACGTGTGCTCTTCCGATCTCA | 468 |
| | CTGTACTTGCCCTGACCA | |
| HEK3-off2_fwd | CACTCTTTCCCTACACGACGCTCTTCCGAT | 469 |
| | CTTTGGTGTTGACAGGGAGCAA | |
| HEK3-off2_rev | GGAGTTCAGACGTGTGCTCTTCCGATCTCT | 470 |
| | GAGATGTGGGCAGAAGGG | |
| HEK3-off2_fwd | CACTCTTTCCCTACACGACGCTCTTCCGAT | 471 |
| | CTTTGGTGTTGACAGGGAGCAA | |
| HEK3-off2_rev | GGAGTTCAGACGTGTGCTCTTCCGATCTCT | 472 |
| | GAGATGTGGGCAGAAGGG | |
| HEK3-off3_fwd | CACTCTTTCCCTACACGACGCTCTTCCGAT | 473 |
| | CTTGAGAGGGAACAGAAGGGCT | |
| HEK3-off3_rev | GGAGTTCAGACGTGTGCTCTTCCGATCTGT | 474 |
| | CCAAAGGCCCAAGAACCT | |

Sequences

| SEQ ID | |
|---|---|
| NO | Nuclease Sequence |
| 1 | MKKSYSIGLDIGTNSVGWSVITDDYKVPAKKMKVLGNTDKKYIKKNLLGALLFDSGETAEATRLK |
| | RTARRRYTRRKNRLRYLQEIFAKEMAKVDESFFQRLEESFLTDDDKNFDSHPIFGNKAEEDAYHDE |
| | FPTIYHLRQYLADSPQKADLRLVYLALAHMIKFRGHFLIEGQLNAENTDVQTLFNDFVEVYDKTVE |
| | ESHLSEITVDALSILTEKVSKSRRLENLVKCYPTEKKNTLFGNLIALSLGLQPNFKTNFKLSEDAKLQ |
| | FSKDTYEEDLEELLGKIGDEYADLFASAKNLYDAILLSGILTVDDNTTKAPLSASMVKRYAEHHED |
| | LEKLKEFVKANKPELYHDIFKDETKNGYAGYIENGVKQDEFYKYLKNTLSKIAGSDYFLDKIERED |
| | FLRKQRTFDNGSIPHQIHLQEMHAILRRQGEYYPFLKENQAKIEKILTFRIPYYVGPLARKNSRFAW |
| | AEYHSDEKITPWNFEEIVDKEESATAFIHRMTNNDLYLPEEKVLPKHSLIYEEFTVYNELTKVKYVT |
| | DQGKAYYFDANMKQEIFDGVFKEHRKVTKDKLMNFLEKEFDEFRIVDLTGLDKENKSFNASLGTY |
| | HDLKKILDKSFLDDKTNETIIEDIIQTLTLFEDRDMIRQRLQKYSDIFTPQQLKKLERRHYTGWGRLS |
| | YKLINGIRNKENGKTILDYLIDDGYANRNFMQLINDDTLPFKQIIQEAQIIGDIDDIETVVHDLPGSPA |
| | IKKGILQSVKIVDELVKVMGHNPDNIVIEMARENQTTNRGRSQSQQRLKKLQESLKELGSNILNEEK |
| | PSYVEDKVENSHLQNDKLFLYYIQNGKDMYTGEELDIDHLSDYDIDHIIPQAFIKDNSIDNRVLTSS |
| | AKNRGKSDDVPSLDIVHDRKADWIRLYKSGLISKRKFDNLTKAEHGGLTEDDKAGFIKRQLVETR |
| | QITKHVAQILDSRENTERDENDKVIRDVKVITLKSNLVSQFRKDFELHKVREINDYHHAHDAYLNA |
| | VIGNALLVKYPQLEPEFVYGKFKHFHGHKENKATAKKFFYSNIMALFKKDVEYSDDTGEMVWNK |
| | EKDFATVRKVLSCPQVNIVKKTEVQTGGFSKESILPKRNSDKLIARKKDWDPKKYGGFDSPTVAYS |
| | VLVVAKVEKGKSKKLKSVKELLGITIMERSSFEKNPIDFLEAKGYKEVKKDLIIKLPKYSLFELENG |
| | RKRMLASAGELQKGNELALPSKYVNFLYLASHYEKLKGSPEDNEQKQLFVEQHKHYLDEIIEQISE |
| | FSKRVILADANLDKVLSAYNKHRDKPIREQAENIIHLFTLTNLGAPAAFKYFDTTIDRKRYTSTKEV |
| | LDATLIHQSITGLYETRIDLSQLGGD |
| 2 | MKKSYSIGLDIGTNSVGWSVVTDDYKVPAKKMKVFGNTDKKYIKKNLLGALLFDSGETAEGTRL |
| | KRTARRRYTRRRNRILYLQEIFAKEMAKVDESFFQRLEESFLTDDDKNFDSHPIFGNKAEEDAYHE |
| | QFPTIYHLRKHLADSSEKADLRLVYLALAHMIKFRGHFLIEGQLKAENTNVQTLFNDFVEVYDKTV |
| | EESHLAEITVDALSILTEKVSKSRRLENLLKYYPTEKKNTLFGNLIALSLGLQPNFKTNFKLSEDAKL |
| | QFSKDTYEEDLEELLGKIGDEYADLFTSAKNLYDAILLSGILTVDDKSTKAPLSASMLKRYVEHHE |
| | DLEKLKDFVKANKPELYHDIFKDETKNGYAGYIENGVKQDEFYKYLKNTLSKIAGSDYFLDKIERE |
| | DFLRKQRTFDNGSIPHQVHLQEMRAILRRQGEYYPFLKENQAKIEKILTFRIPYYVGPLARKDSRFA |
| | WAEYHSDEKITPWNFDKVIDKESSARAFIEHMTNNDLYLPEEKVLPKHSPLYEKYTVYNELTKVK |
| | YVTEQGKSSFFDANMKQEIFEHVFKKNRKVTKDKFLNYLNKEFPEYRIHDLIGLDKENKSFNASLG |
| | TYHDLKKILDKSFLDNKENEEIIEDVVLTLTLFEDRDMIRQRLQKYSDIFTKDQLKKLERRHYTGW |
| | GRLSYKLINGIRDKQSNKTILDYLLEDDGVKKNINRNFMQLINDDSLSFKTIIQEAQVIGDIDDIKAV |
| | VHDLPGSPAIKKGILQSVKIVDELVKVMGHNPDNIVIEMARENQTTNRGRSQSQQRLKKLQESLKE |
| | LGSNILNEEKPSYIEDKVENSHLQNDRLFLYYIQNGKDMYTGDELDIDHLSDYDIDHIIPQAFIKDNS |
| | IDNRVLTSSAKNRGKSDDVPSLDIVRERKADWIRLYKAGLISKRKFDNLTKAERGGLTENDKAGFI |
| | KRQLVETRQITKHVAQILDARFNTKRDENDKVIRDVKIITLKSNLVSQFRKEFGFYKVREINDYHHA |
| | HDAYLNAVVGTALIKKYPKLESEFVYGDYKVYDVRKMIAKSEQEIGKATAKRFFYSNIMNFFKTEI |
| | KLANGEIRKRPLIETNEETGEVVWNKERDFAIVRKVLSCPQVNIVKKTEVQTGGFSKESILPKRNSD |
| | KLIARKKDWDPKKYGGFDSPTVAYSVLVVAKVEKGKSKKLKSVKELLGITIMERSSFEKNPIDFLE |
| | AKGYKEVKKDLIIKLPKYSLFELENGRKRMLASAGELQKGNELALPSKYVNFLYLASHYEKLKGSP |
| | EDNEQKQLFVEQHKHYLDEIIEQISEFSKRVILADANLDKVLSAYNKHRDKPIREQAENIIHLFTLTN |
| | LGAPAAFKYFDTTIDRKRYTSTKEVLDATLIHQSITGLYETRIDLSQLGGD |
| 3 | MNKPYSIGLDIGTNSVGWSVVDDDYKVPAKKMRVFGDTDKGHIKKNLLGTLLFDSGETAESRRLK |
| | RTARRRYTRRRNRILYLQEIFAKEMAKVDESFFHRLDESFLTDDDKTFDSHPIFGNKAEEDAYHQQ |
| | FPTIYHLRKHLADSTEKADLRLVYLALAHMIKFRGHFLIEGELNAENTDVQKLFADFVGVYDRTFD |
| | DSHLSEITVDASSILTEKISKSRRLEKLIKHYPTEKKNTLFGNLVALALGLQPNFKTNFKLSEDAKLQ |
| | FSKDTYEEDLEELLGKIGDDYADLFTSAKNLYDAILLSGILTVDDESTKAPLSASMIKRYAEHHEDL |
| | EKLKEFVKANKPELYHDIFKDESKNGYAGYIENGVKQDEFYKYLKETLSKQDGSQYFLDKIEREDF |
| | LRKQRTFDNGSIPHQLHLKEMRSILHRQGEYYPFLKENQAKIEKILTFRIPYYVGPLARKDSRFAWA |
| | EYHSDEPITPWNFDEVVDKEKSAEKFITRMTLNDLYLPEEKVLPKHSHVYETYTVYNELTKVKYV |
| | NEQGEAKFFDANMKQEIFDHVFKENRKVTKDKLMDFLGKEFDEFRIVDITGLDKENKAFNASLGT |
| | YHDLKKILDKSFLDDKENETIIEDIIQTLTLFEDRDMIRQRLQKYSDIFTPQQLKKLERRHYTGWGRL |
| | SYKLINGIRDKQTGKTILDYLIDDGYANRNFMQLINDDTLPFKEIIQKAQIIGDIDDIKAVVHDLPGSP |
| | AIKKGILQSVKIVDELVKVMGHNPDNIVIEMARENQTTNRGRTQSQQRLKKLQESLKELGSNILNE |
| | KKPSYVEDKVENTHLQNDRLFLYYIQNGKDMYTGDELDIDHLSDYDIDHIIPQAFIKDDSIDNRVLT |
| | SSAKNRGKSDDVPSIEIVRDRKADWMRLRKAGLISKRKFDNLTKAERGGLTEADKAGFIKRQLVET |
| | RQITKHVAQILDSRENTERDENDKVIREVKVITLKSNLVSQFRKDFQFYKVREINDYHHAHDAYLN |
| | AVVGTALIKKYPKLESEFVYGDYKVYDVRKMIAKSEQEIGKATAKRFFYSNIMNFFKTEIKLANGE |
| | IRKRPLIETNEETGEIVWNKEKDFATVRKVLSCPQVNIVKKTEVQTGGFSKESILPKRNSDKLIARKK |
| | DWDPKKYGGFDSPTVAYSVLVVAKVEKGKSKKLKSVKELLGITIMERSSFEKNPIDFLEAKGYKEV |
| | KKDLIIKLPKYSLFELENGRKRMLASAGELQKGNELALPSKYVNFLYLASHYEKLKGSPEDNEQKQ |
| | LFVEQHKHYLDEIIEQISEFSKRVILADANLDKVLSAYNKHRDKPIREQAENIIHLFTLTNLGAPAAF |
| | KYFDTTIDRKRYTSTKEVLDATLIHQSITGLYETRIDLSQLGGD |
| 4 | MKKPYSIGLDIGTNSVGWAVVTDDYKVPAKKMKVLGDTDKSHIKKNLLGTLLFESGETAEGRRLK |
| | RTARRRYTRRRNRILYLQEIFAEEMNKVDDSFFQRLDESFLKDDDKNFDSHPIFGNKAEEDAYHQK |
| | FPTIYHLRQYLADSPEKADLRLVYLALAHIIKYRGHELFEGDLKSENKDVQHLFNDFVEMEDKTVE |
| | GSYLSENLPNVADVLVEKVSKSRRLENVLHYFPNEKKNGLFGNFIALALGLQPNFKTNFKLSEDAK |
| | LQFSKDTYDEDLEELLGKIGDDYADLFVAAKNLYDAILLSGILTVTDPTTKAPLSASMIDRYEKHH |
| | KDLALLKNLIKQNLTQEKYDEVFSDESKNGYAGYISGKVKQEEFYKYLKNTLSKIEGSDYFLDKIE |
| | REDFLRKQRTFDNGSIPHQIHLQEMNAIIRRQGEHYPFLKENKEKIEKILTFRIPYYVGPLARGNRDF |
| | AWLTRNSDQAIRPWNFEEIVDQASSAEDFINKMTNYDLYLPEEKVLPKHSLLYETFAVYNELTKVK |
| | FIAEGLRDYQFLDSGQKKQIVNQLFKEKRKVTEKDIIQYLHNVDGYDGIELKGIEKQFNASLSTYHD |
| | LLKIIKDKEFMDDPKNEEILENIVHTLTIFEDREMIKQRLAQYDSLFDEKVIKALTRRHYTGWGKLS |
| | AKLINGIYDKQSKKTILDYLIDDGEINRNFMQLINDDGLSFKDIIQKAQVVGKTNDVKQVVQELPGS |
| | PAIKKGILQSIKLVDELVKVMGHAPESIVIEMARENQTTARGKKNSQQRYKRIEDALKNLAPGLDS |
| | NILKENPTDNIQLQNDRLFLYYLQNGKDMYTGEALDINQLSNYDIDHIVPQAFIKDDSLDNRVLTSS |
| | KDNRGKSDNVPSIEVVQKRKAFWQQLLDSKLISERKENNLTKAERGGLDERDKVGFIKRQLVETR |
| | QITKHVAQILDARFNTEVDENNQRIRKVKIITLKSNLVSNFRKEFGLYKVREINDYHHAHDAYLNA |
| | VVAKAILKKYPKLEPEFVYGDYQKYDLKRYISRSKDPKDIEKATEKYFFYSNLLNFFKEEVHYADG |
| | TIIKRENIEYSNDTGEIAWNKEKDFATVRKVLSCPQVNIVKKTEVQTGGFSKESILPKRNSDKLIARK |
| | KDWDPKKYGGFDSPTVAYSVLVVAKVEKGKSKKLKSVKELLGITIMERSSFEKNPIDFLEAKGYKE |
| | VKKDLIIKLPKYSLFELENGRKRMLASAGELQKGNELALPSKYVNFLYLASHYEKLKGSPEDNEQK |
| | QLFVEQHKHYLDEIIEQISEFSKRVILADANLDKVLSAYNKHRDKPIREQAENIIHLFTLTNLGAPAA |
| | FKYFDTTIDRKRYTSTKEVLDATLIHQSITGLYETRIDLSQLGGD |
| 5 | MDKKYSIGLDIGTNSVGWAVITDDYKVPAKKMKVLGNTDKRTIKKNLLGALLFDSGETAEATRLK |
| | RTARRRYTRRKNRLRYLQEIFSKEMSNVDANFFYRLEDSFLVPEDKQQERHPIFGNLVEEIEYHKEF |
| | PTIYHLRKQLADSKEKADLRLIYLALAHMIKYRGHELFEGDLKSENTDVQRLFKDFVEVYDKTVEG |
| | SYLSENLTNVTDVLVEKISKSRRLENILHYFPNEKKNGLFGNFLALALGLQPNFKNNFDLAEEAKL |
| | QISKDTYEEELEVLLAQIGDNYAELFIATKELYDGILLAGILTVTDSSTKAPLSASMIERYENHQGDL |
| | AKLKAFIRRFCKETYDEVFSDEVKDGYAGYIEGKTSQEDFYVHLKKLLANLEGADYFLEKINREDL |
| | LRKQRTFDNGSIPYQIHLVEMRAILDKQAKFYPFLAKNQDRIEKLLTFRIPYYVGPLARGKSDFAWL |
| | SRKSADKITPWNFDEIVDKESSAEAFINRMTNYDLYLPNQKVLPKHSLLYEKFTVYNELTKVKYKT |
| | EQGKTAFFDANMKQEIFDGVFKVYRKVTKDKLMDFLEKEFDEFRIVDLMGLDKENKAFNASYGT |
| | YHDLRKILDKDFLDNSDNEKILEDIVLTLTLFEDREMIRKRLEKYKDIFTEKQRKQLERRHYTGWG |
| | RLSAKLIHGIRNKESRKTILDYLIDDGYSNRNFMQLINDDTLTFKEEIAKAQVVGETDSLKQFISELA |
| | GSPAIKKGILQTVKIVDELVKVMGHKPENIVIEMARENQTTQKGQKNSQERMKRIEEGIKELGSQIL |
| | KEHPVENTQLQNEKLYLYYLQNGRDMYVDQELDINRLSDYDIDHIIPQAFIKDDSIDNRVLTSSKD |
| | NRGKSDNVPSEDVVKKMKNYWRQLLNAKLISQRKFDNLTKAERGGLSELDKVGFIKRQLVETRQI |
| | TKHVAQILDSRMNTKYDENDKLIRDVKIITLKSKLVSDFRKDFQFYKVREINNYHHAHDAYLNAV |
| | VGTALIKKYPKLESEFVYGDYKVYDVQKMIAKSEQEIGKATAKFFFYSNIMNFFKTEITLANGEIRK |
| | RPMIETNEETGEIVWDKGRDFATVRKVLCMPQVNIVKKTEVQTGGFSKESILPKRNSDKLIARKKD |
| | WDPKKYGGFDSPTVAYSVLVVAKVEKGKSKKLKSVKELLGITIMERSSFEKNPIDFLEAKGYKEVK |
| | KDLIIKLPKYSLFELENGRKRMLASAGELQKGNELALPSKYVNFLYLASHYEKLKGSPEDNEQKQL |
| | FVEQHKHYLDEIIEQISEFSKRVILADANLDKVLSAYNKHRDKPIREQAENIIHLFTLTNLGAPAAFK |
| | YFDTTIDRKRYTSTKEVLDATLIHQSITGLYETRIDLSQLGGD |
| 6 | MTKPYSIGLDIGTNSVGWAVITDSYKVPSKKMKVLGNTSKKYVKKNLLGALLFESGITAEGRRLK |
| | RTARRRYTRRRNRILYLQEIFSTEMSKIDESFFQRLDDSFLVPDDKRDSKYPIFGNLVEEKTYHDEFP |
| | TIYHLRKYLADSSKKADLRLVYLALAHMIKYRGHFLIEGDFNSRNNDIKKNFQDFLDSYNAIFESDL |
| | SLENSKQLEEIVKDKISKSAKKDRILKLFPGEKNSGIFSEFLKLIVGNQADFKKYFNLDDQASLHFSK |
| | ESYDEDLETLLGHIGDDYSDVFLKAKKLYDAILLSGILTVTDNETEAPLSSAMIMRYKEHEEDLALL |
| | KAYIRNISLETYNEVFKDDTKNGYAGYIDGKTNQEDFYVYLKKLLTGLEGADYFLEKINREDLLRK |
| | QRTFDNGSIPYQIHLQEMRAIIDKQAKFYPFLAKNKERIEKILTFRIPYYVGPLARGNSDFAWSIRKR |
| | NEKITPWNFEDVIDKESSAEAFINRMTSFDLYLPEEKVLPKHSLLYETFTVYNELTKVRFIAESMRD |
| | YQFLDSKQKKDIVRLYFKGKRKVTDKDIIDYLHAIDGYDGIELKGIEKQFNSSLSTYHDLLNIINDKE |
| | FLDDASNEAIIEEIIHTLTIFEDREMIKQRLSKFENIFDKSVLKKLSRRHYTGWGKLSAKLINGIRDEK |
| | SGKTILDYLIDDGISNRNFMQLIHDDTLSFKKKIQKAQIIGDQDNIKQVVQSLPGSPAIKKGILQSIKI |
| | VDELVKVMGRNPESIVVEMARENQYTNQGKSNSQQRLKRLEESLKELGSKILKENVPAKLSKIDN |
| | NALQNERLYLYYLQNGKDMYTGKDLDIDRLSNYDIDHIIPQAFLKDNSIDNKVLVSSASNRGKSDD |
| | VPSLDVVKKRKGFWHQLLKSKLISQRKFDNLTKAERGGLTEDDKAGFIKRQLVETRQITKHVAQIL |
| | DSRFNTKRDDNDKVIRDVKIITLKSNLVSQFRKEFGLYKVREINDYHHAHDAYLNAVVATAILKKY |
| | PKLEPEFVYGDYQKYDIKKYLASKDDSSLGKATAKMFFYSNLMNFFKKKVRLSDGTVLIRPVIEYS |
| | KDTGEIAWNKEKDFATVRKVLSCPQVNIVKKTEVQTGGFSKESILPKRNSDKLIARKKDWDPKKY |
| | GGFDSPTVAYSVLVVAKVEKGKSKKLKSVKELLGITIMERSSFEKNPIDFLEAKGYKEVKKDLIIKL |
| | PKYSLFELENGRKRMLASAGELQKGNELALPSKYVNFLYLASHYEKLKGSPEDNEQKQLFVEQHK |
| | HYLDEIIEQISEFSKRVILADANLDKVLSAYNKHRDKPIREQAENIIHLFTLTNLGAPAAFKYFDTTID |
| | RKRYTSTKEVLDATLIHQSITGLYETRIDLSQLGGD |
| 7 | MTKPYSIGLDIGTNSVGWAVITDSYKVPSKKMKVLGNTSKKYVKKNLLGALLFESGITAEGRRLK |
| | RTARRRYTRRRNRILYLQEIFSTEMAKIDESFFQRLDDSFLVPDDKRDSKYPIFGNLVKEKTYHDEF |
| | PTIYHLRKYLADSSKKADLRLVYLALAHMIKYRGHFLIEGDFNSRNNDIKKNFQNFLDSYNAIFESD |
| | LSLENSKQLEEIVKDKISKSAKRERVLKLFPDEKSTGLFSEFLKLIVGNQADFKKHFDLEEKAPLQFS |
| | KDTYDEDLENLLGQIGDDFADLFLVAKKLYDAILLSGILTVTDPSTKAPLSASMIERYENHQKDLSA |
| | LKQFIRSNLSEKYDEVFSDQSKDGYAGYINGKTTQEAFYKYIKNLLSKFEGADYFLDKIEREDFLRK |
| | QRTFDNGSIPHQIHLQEMNAIIRRQGEHYPFLKENKEKIEKILTFRIPYYVGPLARGNRDFAWLTRNS |
| | DQAIRPWNFEEVVDKASSAEAFINKMTNFDLYLPEEKVLPKHSLLYETFAVYNELTKVKFIAEGLR |
| | DYQFLDSGQKKQIVNQLFKEKRKVTEKDIIQFLHNVDGYDGIELKGIEKQFNASLATYHDLLKIIKD |
| | KEFMDDPKNEEILENIIHTLTIFEDREMIKQRLAQYDSLFDEKVIKALTRRHYTGWGKLSAKLINGIC |
| | DKQTGKTILDYLIDDGFANRNFMQLINDDTLPFKQIIQKAQIVGDVDDIEQVVHNLPGSPAIKKGIL |
| | QSIKIVDELVKVMGHNPDNIVIEMARENQTTNRGRSQSQQRLKKLQESLKELGSNILNEEKPSYVES |
| | KVENSQLQNDRLFLYYIQNGKDMYTGEELDIDRLSDYDIDHIIPQAFIKDDSIDNRVLTSSAKNRGK |
| | SDDVPSLEVVKKRKTYWHQLLKAKLISQRKFDNLTKAERGGLTEKDKAGFIKRQLVETRQITKHV |
| | AQILDSRFNTKRDENDKVIRDVKVITLKSNLVSQFRKDFGFYKVREINDYHHAHDAYLNAVVGTA |
| | LLKKYPKLAPEFVYGEYKKYDVRKIVRSKDESSLGKATAKMFFYSNLMNFFKKEVRLSDGTVIVR |
| | PVIEYSKDTGEIAWNKEKDFATVRKVLSCPQVNIVKKTEVQTGGFSKESILPKRNSDKLIARKKDW |
| | DPKKYGGFDSPTVAYSVLVVAKVEKGKSKKLKSVKELLGITIMERSSFEKNPIDFLEAKGYKEVKK |
| | DLIIKLPKYSLFELENGRKRMLASAGELQKGNELALPSKYVNFLYLASHYEKLKGSPEDNEQKQLF |
| | VEQHKHYLDEIIEQISEFSKRVILADANLDKVLSAYNKHRDKPIREQAENIIHLFTLTNLGAPAAFKY |
| | FDTTIDRKRYTSTKEVLDATLIHQSITGLYETRIDLSQLGGD |
| 8 | MTKPYSIGLDIGTNSVGWAVITDSYKVPSKKMKVLGNTSKKYVKKNLLGALLFESGITAEGRRLK |
| | RTARRRYTRRRNRILYLQEIFSTEMAKIDESFFQRLDDSFLVPDDKRDSKYPIFGNLVKEKTYHDEF |
| | PTIYHLRKYLADSSKKADLRLVYLALAHMIKYRGHFLIEGDFNSRNNDIKKNFQDFLDSYNAIFESD |
| | LSLENSEQLEEIVKDKISKSAKRERVLKLFPDEKSTGLFSEFLKLIVGNQADFKKHFDLEEKAPLQFS |
| | KDTYDEDLENLLGQIGDDFADLFLVAKKLYDAILLSGILTVTDPSTKAPLSASMIERYENHQKDLAT |
| | LKQFVRTNLPEKYDEVFSDQSKDGYAGYIDGKTTQEAFYKYIKNLLSKFEGADYFLDKIEREDFLR |
| | KQRTFDNGSIPHQIHLQEMNAIIRRQGEHYPFLKENKEKIEKILTFRIPYYVGPLARGNRDFAWLTR |
| | NSDQAIRPWNFEEIVDQASSAEDFINKMTNYDLYLPEEKVLPKHSLLYETFAVYNELTKVKFIAEGL |
| | RDYQFLDSGQKKQIVNQLFKEKRKVTEKDIIHYLHNVDGYDGIELKGIEKQFNASLSTYHDLLKIIK |
| | DKEFMDDPKNEEILENVVHTLTIFEDREMIKQRLAQYDSLFDEKVIKALTRRHYTGWGKLSAKLIN |
| | GICDKQTGKTILDYLIDDGEINRNFMQLIHDDGLSFKDIIQKAQVVGKTDDVKQVVQELPGSPAIKK |
| | GILQSIKIVDELVKVMGHNPENIVVEMARENQFTNQGRRNSQQRYKKIEDAIKNLDSNLNSKILKE |
| | YPTNNQALQNDRLFLYYLQNGKDMYTGEELDINQLSNYDIDHIVPQAFIKDDSLDNRVLTSSKDNR |
| | GKSDNVPSLEVVQKRKAFWQQLLDSKLISERKENNLTKAERERGGLNELDKVGFIKRQLVETRQIT |
| | KHVAQILDARFNTEVNEKGQRIRKVKIITLKSNLVSNFRKEFGLYKVREINDYHHAHDAYLNAVVA |
| | KAILKKYPKLEPEFVYGDYQKYDVKRYISRSKDPKEIEKATEKYFFYSNLLNFFKEEVHYADGTIIK |
| | RENIEYSKDTGEIAWNKEKDFATVRKVLSCPQVNIVKKTEVQTGGFSKESILPKRNSDKLIARKKD |
| | WDPKKYGGFDSPTVAYSVLVVAKVEKGKSKKLKSVKELLGITIMERSSFEKNPIDFLEAKGYKEVK |
| | KDLIIKLPKYSLFELENGRKRMLASAGELQKGNELALPSKYVNFLYLASHYEKLKGSPEDNEQKQL |
| | FVEQHKHYLDEIIEQISEFSKRVILADANLDKVLSAYNKHRDKPIREQAENIIHLFTLTNLGAPAAFK |
| | YFDTTIDRKRYTSTKEVLDATLIHQSITGLYETRIDLSQLGGD |
| 9 | MNKPYSIGLDIGTNSVGWAVVDDDYKVPAKKMRVFGDTDRSHIKKNLLGTLLFDSGETAEGRRL |
| | KRTARRRYTRRRNRILYLQEIFAEEMSKVDDSFFHRLDESFLTDDDKNFDSHPIFGNKAEEDAYHQ |
| | KFPTIYHLRKHLADSTEKADLRLVYLALAHMIKFRGHFLIEGQLNAENTDVQKLFADFVGVYDRTF |
| | DDSHLSEITVDASSILTEKISKSRRLENLITHYPKEKKNTLFGNLIALALGLQPNFKTNFKLSEDAKL |
| | QFSKDTYEEDLEELLGKIGDDYADLFTSAKNLYDAILLSGILTVDDESTKAPLSASMVKRYAEHHE |
| | DLEKLKEFIKANKPELYHDIFKDETKNGYAGYIENGVKQDEFYKYLKNTLSKIAGSDYFLDKIERE |
| | DFLRKQRTFDNGSIPHQIHLQEMHAILRRQGDYYPFLKENQDRIEKILTFRIPYYVGPLARKDSRFA |
| | WANYHSEEKITPWNFDKVIDKEKSAEKFITRMTSNDLYLPEEKVLPKHSLLYETYTVYNELTKVKY |
| | VNEQGNATFFDSNIKQEIFEQLFKTKRKVTKKDIINYLENAYCIDDVEIMGVEDRFNASLGTYHDLL |
| | KIIQDKSFLDDKKNEEIIEDVVLTLTLFEDRDMIRQRLQKYSDIFTKQQLKKLERRHYTGWGRLSYK |
| | LINGIRNKENNKTILDFLIDDGRANRNFMQLINDDSLSFKTIIQEAQVVGDVDDIETVVHDLPGSPAI |
| | KKGILQSIKIVDELVKVMGHNPDNIVIEMARENQTTNRGRSQSQQRLKKLQESLKELGSSILNKEKP |
| | SYIEDKVENSHLQNDKLFLYYIQNGKDMYTGEELDIDHLSDYDIDHIIPQAFIKDDSIDNRVLTSSAK |
| | NRGKSDDVPSLDIVRDRKAEWIRLYKSKLISKRKFDNLTKAERGGLTENDKAGFIKRQLVETRQIT |
| | KHVAQILDARFNTERDENDKVIRDVKVITLKSNLVSQFRKEFGFYKVREINDYHHAHDAYLNAVV |
| | GTALLKKYPKLAPEFVYGEYQKYDVRKFISTKDNKDIGKATAKYFFYSNLLNFFKEEVRYANGTII |
| | VRPVIEYSKDTGEIAWNKEKDFATVRKVLSCPQVNIVKKTEVQTGGFSKESILPKRNSDKLIARKKD |
| | WDPKKYGGFDSPTVAYSVLVVAKVEKGKSKKLKSVKELLGITIMERSSFEKNPIDFLEAKGYKEVK |
| | KDLIIKLPKYSLFELENGRKRMLASAGELQKGNELALPSKYVNFLYLASHYEKLKGSPEDNEQKQL |
| | FVEQHKHYLDEIIEQISEFSKRVILADANLDKVLSAYNKHRDKPIREQAENIIHLFTLTNLGAPAAFK |
| | YFDTTIDRKRYTSTKEVLDATLIHQSITGLYETRIDLSQLGGD |
| 10 | MKKPYSIGLDIGTNSVGWAVVTDDYKVPAKKMKVLGNTNKESIKKNLIGALLFDAGNTAADRRL |
| | KRTARRRYTRRRNRILYLQEIFAEEMNKVDESFFHRLDDSFLVPEDKRGSKYPIFATLEEEKEYHKK |
| | FPTIYHLRKHLADSKEKADLRLIYLALAHMIKYRGHFLYEESFDIKNNDIQKIFNEFISIYDNTFEGSS |
| | LSGQNAQVEAIFTDKISKSAKRERVLKLFPDEKSTGLFSEFLKLIVGNQADFKKHFDLEEKAPLQFS |
| | KDTYDEDLENLLGQIGDDFADLFLVAKKLYDAILLSGILTVTDPSTKAPLSASMIERYENHQKDLA |
| | ALKQFIQNNLQEKYDEVFSDQSKDGYAGYIDGKTTQEAFYKYIKNLLSKLEGADYFLDKIEREDFL |
| | RKQRTFDNGSIPHQIHLQEMNAIIRRQGEHYPFLKENKEKIEKILTFRIPYYVGPLARGNRDFAWLTR |
| | NSDQAIRPWNFEEIVDQASSAEDFINKMTNYDLYLPEEKVLPKHSLLYETFAVYNELTKVKFIAEG |
| | MRDYQFLDSGQKKKIVSQLFEDKRKVTEKDIIHYLHTIDGYDGIELSGIEKQFNASLSTYHDLLKIIK |
| | DKAFLDDSENEAIIEEIIHTLTIFEDREMIKQRLAQYADIFDKSVLKKLARRHYTGWGKLSAKLINGI |
| | RDKQSNKTILDYLIDDGKINRNFMQLINDDSLSFKETIQKAQVVGDTDDVKQVVQELPGSPAIKKGI |
| | LQSIKIVDELVKVMGHNPDNIVIEMARENQTTNRGRSQSQQRLKKLQDSLKELGSNILNEEKPSYIE |
| | DKVENSHLQNDQLFLYYIQNGKDMYTGEELDIDHLSDYDIDHIIPQAFIKDDSIDNRVLTSSAKNRG |
| | KSDDVPSLDIVRERKADWIRLYKSKLISKRKFDNLTKAERGGLTEADKAGFIKRQLVETRQITKHV |
| | AQILDSRFNTKRDENDKVIRDVKVITLKSNLVSQFRKDFELYKVREINDYHHAHDAYLNAVVGTA |
| | LLKKYPKLAPEFVYGEYKKYDIRKFITSSKDKATAKYFFYSNLMNFFKRIIRYSNGKIVVRPVVEYS |
| | KDTGEIAWNKEKDFATVRKVLSCPQVNIVKKTEVQTGGFSKESILPKRNSDKLIARKKDWDPKKY |
| | GGFDSPTVAYSVLVVAKVEKGKSKKLKSVKELLGITIMERSSFEKNPIDFLEAKGYKEVKKDLIIKL |
| | PKYSLFELENGRKRMLASAGELQKGNELALPSKYVNFLYLASHYEKLKGSPEDNEQKQLFVEQHK |
| | HYLDEIIEQISEFSKRVILADANLDKVLSAYNKHRDKPIREQAENIIHLFTLTNLGAPAAFKYFDTTID |
| | RKRYTSTKEVLDATLIHQSITGLYETRIDLSQLGGD |
| 11 | MKKSYSIGLDIGTNSVGWAVVDDDYKVPAKKMRVFGDTDRSHIKKNLLGTLLFDSGETAESRRLK |
| | RTARRRYTRRRNRILYLQEIFAEEMAKVDDSFFHRLDESFLTDDDKTFDSHPIFGNKAEEDAYHQK |
| | FPTIYHLRKHLADSTEKADLRLVYLALAHMIKFRGHFLIEGQLNAENTDVQTLFNDFVEVYDKTVE |
| | ESHLAEITVDALSILTEKVSKSRRLENLIKYYPTEKKNTLFGNLVALALGLQPNFKTNFKLSEDAKL |
| | QFSKDTYEEDLEELLGKIGDEYADLFTSAKNLYDAILLSGILTVDDNSTKAPLSASMIKRYEEHHED |
| | LEKLKTFVKANNSDKYHEIFSDKDKNGYAGYIENGVKQDEFYKYLKNTLSKIAGSDYFLDKIERED |
| | FLRKQRTFDNGSIPHQIHLQEMHAILRRQGDYYPFLKENQDRIEKILTFRIPYYVGPLARKDSRFAW |
| | ANYHSDEPITPWNFDEVVDKEKSAEKFITRMTLNDLYLPEEKVLPKHSHVYETFAVYNELTKIKYV |
| | NEQGKSFFFDANMKQEIFDHVFKENRKVTKDKLLDFLNKEFEEFRIVDLIGLDKENKSFNASLGTY |
| | HDLKKILDKSFLDDKTNEQIIEDIVLTLTLFEDRDMIRQRLQKYSDIFTKQQLKKLERRHYTGWGRL |
| | SYKLINGIRNKENNKTILDFLIDDGRANRNFMQLINDDSLSFKTIIQEAQVVGDVDDIEAVVHDLPG |
| | SPAIKKGILQSVKIVDELVKVMGHNPDNIVIEMARENQTTNRGRVQSQQRLKKLQESLKELGSEILN |
| | VEKPSYVEGKVENNHLQNDKLFLYYIQNGKDMYTGEELDIDHLSDYDIDHIIPQAFIKDDSIDNRVL |
| | TSSAKNRGKSDDVPSLEIVHDRKADWIRLYKSGLISKRKFDNLTKAERGGLTEADKAGFIKRQLVE |
| | TRQITKHVAQILDARFNTERDENDKVIRDVKVITLKSNLVSQFRKDFKFYKVREINDYHHAHDAYL |
| | NAVVGTALLKKYPKLAPEFVYGEYKKYDIRKFITNSSDKATAKYFFYSNLLNFFKTKVRYADGTIL |
| | VRPVIEYSKDTGEIAWNKEKDFATVRKVLSCPQVNIVKKTEVQTGGFSKESILPKRNSDKLIARKKD |
| | WDPKKYGGFDSPTVAYSVLVVAKVEKGKSKKLKSVKELLGITIMERSSFEKNPIDFLEAKGYKEVK |
| | KDLIIKLPKYSLFELENGRKRMLASAGELQKGNELALPSKYVNFLYLASHYEKLKGSPEDNEQKQL |
| | FVEQHKHYLDEIIEQISEFSKRVILADANLDKVLSAYNKHRDKPIREQAENIIHLFTLTNLGAPAAFK |
| | YFDTTIDRKRYTSTKEVLDATLIHQSITGLYETRIDLSQLGGD |
| 12 | MKKPYSIGLDIGTNSVGWAVVDDDYKVPAKKMRVFGDTDRSHIKKNLLGTLLFDDGNTAESRRL |
| | KRTARRRYTRRRNRILYLQEIFAEEMNKVDESFFHRLDDSFLVPEDKRGSKYPIFATLEEEKEYHKK |
| | FPTIYHLRKHLADSKEKTDLRLIYLALAHMIKYRGHFLYEESFDIKNNDIQKIFNEFISLYDNTFEGSS |
| | LSGQNAQVEAIFTDKISKSAKRERVLKLFPDEKSTGLFSEFLKLIVGNQADFKKHFDLEEKAPLQFS |
| | KDTYDEDLENLLGQIGDDFADLFLVAKKLYDAILLSGILTVTDPSTKAPLSASMIERYENHEEDLAA |
| | LKQFVKANLPEKYDEVFSDQSKDGYAGYIDGKTTQEAFYKYIKNLLSKLEGADYFLDKIEREDFLR |
| | KQRTFDNGSIPHQIHLQEMNAILRRQGEHYPFLKDNKEKIEKILTFRIPYYVGPLARGNRDFAWLTR |
| | NSDQAIRPWNFEEVVDKASSAEDFINKMTNYDLYLPEEKVLPKHSLLYETFAVYNELTKVKFIAEG |
| | LRDYQFLDSGQKKQIVNQLFKTKRKVTEKDIINYLHKVDGYDGVEISGVEDRFNASLGTYHDLLKI |
| | IKDKSFLDDKKNEEIIEDIVLTLTLFEDRDMIRQRLQKYSDIFTPQQLKKLERRHYTGWGRLSYKLIN |
| | GIRNKENNKSILDYLIDDGYANRNFMQLINDDTLPFKQIIQKSQVVGDIDDIKTVVHDLPGSPAIKK |
| | GILQSVKIVDELVKVMGHNPDNIVIEMARENQTTNRGRSQSQQRLKKLQESLKELGSNILNVEKPS |
| | YIEDKVENNHLQNDKLFLYYIQNGKDMYTGEELDIDHLSDYDIDHIIPQAFIKDDSIDNRVLTSSAK |
| | NRGKSDDVPSLDIVRGRKAEWIRLYKAKLISQRKFDNLTKAERGGLTENDKAGFIKRQLVETRQIT |
| | KHVAQILDARFNTKRDENDKLIREVKVITLKSNLVSQFRKEFGFYKVREINDYHHAHDAYLNAVV |
| | GTAILKKYPKLTSEFVYGEYKKYDIRKFIKSDDHSEMGKATAKYFFYSNLLNFFKEEIKYANGKIVV |
| | RPVVEYSKDTGEIAWNKEKDFATVRKVLSCPQVNIVKKTEVQTGGFSKESILPKRNSDKLIARKKD |
| | WDPKKYGGFDSPTVAYSVLVVAKVEKGKSKKLKSVKELLGITIMERSSFEKNPIDFLEAKGYKEVK |
| | KDLIIKLPKYSLFELENGRKRMLASAGELQKGNELALPSKYVNFLYLASHYEKLKGSPEDNEQKQL |
| | FVEQHKHYLDEIIEQISEFSKRVILADANLDKVLSAYNKHRDKPIREQAENIIHLFTLTNLGAPAAFK |
| | YFDTTIDRKRYTSTKEVLDATLIHQSITGLYETRIDLSQLGGD |
| 13 | MKKPYSIGLDIGTNSVGWTVITDDYKVPAKKMKVLGNTDKSHMKKNLLGALLFDSGNTAENRRL |
| | KRTARRRYTRRRNRILYLQEIFAEEMGKVDDSFFHRLEDSFLVVEDKRGERHPIFGNLEEEVKYHE |
| | NFPTIYHLRQYLADNPAKADLRLVYLALAHIIKFRGHFLIEGSFDTRNNDVQRLFSEFLSVYDNVFE |
| | GSSLSEQNAQVEAILTDKISKSAKRERVLKLFPDEKSTGLFAEFLKLIVGNQADFKKHFDLEEKAPL |
| | QFSKDTYDEDLENLLGQIGDDFADLFLVAKKLYDAILLSGILTVTDPSTKAPLSASMIERYESHQKD |
| | LAALKQFIQNNLSDKYDEVFSDQSKDGYAGYIDGKTTQEAFYKYVKKLIEKIEGTDYFLDKIKRED |
| | LLRKQRTFDNGSIPHQIHLGELRAILRRQGDFYPFLKDNQERIEKILTFRIPYYVGPLARGNNDFAWL |
| | TRNSDQAIRPWNFEEVVDKARSAEDFIHRMTSFDLYLPEEKVLPKHSLLYETFTVYNELTKVKFIAE |
| | GLRDYQFLDRGQKKEIVNQLFKEKRKVTKKDIEHYLHTIDGYDGIELKGIEKQFNSSLSTYHDLLKI |
| | IKDKEFLDDASNEAILEEIVHTLTIFEDREMIKQRLAQYSAIFDDKVIKALSRRHYTGWGRLSAKLIN |
| | GIRDEKSGKTILDYLIDDGLANRNFMQLIHDDSLTFVEEIQKAQVSGQGHSLHEQIANLAGSPAIKK |
| | GILQTVKIVDELVKIMGHNPTNIVIEMARENQTTNQGRRNSQQRLKGIEEGMKELGSQILKEHPVEN |
| | SQLQNEKLYLYYLQNGRDMYTGQELDINRLSDYDIDHIVPQSFIKDDSIDNRVLTLSNENRGKSDD |
| | VPSEEVVKKMKNYWRQLLNVKLITQRKFDNLTKAERGGLSELDKAGFIKRQLVETRQITKHVAQI |
| | LDSRMNAKYDENDKLIRDVKIITLKSKLVSDFRKDFQFYKVREINNYHHAHDAYLNAVVGTALIK |
| | KYPKLESEFVYGDYKVYDVRKMIAKSEQEIGKATAKYFFYSNIMNFFKTEITLANGEIRKRPVIEAN |
| | EETGEIVWDKGRDFTMVRKVLCMPQVNIVKKTEVQTGGFSKESILPKRNSDKLIARKKDWDPKKY |
| | GGFDSPTVAYSVLVVAKVEKGKSKKLKSVKELLGITIMERSSFEKNPIDFLEAKGYKEVKKDLIIKL |
| | PKYSLFELENGRKRMLASAGELQKGNELALPSKYVNFLYLASHYEKLKGSPEDNEQKQLFVEQHK |
| | HYLDEIIEQISEFSKRVILADANLDKVLSAYNKHRDKPIREQAENIIHLFTLTNLGAPAAFKYFDTTID |
| | RKRYTSTKEVLDATLIHQSITGLYETRIDLSQLGGD |
| 14 | MDKKYSIGLDIGTNSVGWAVITDDYKVPAKKMKVLGNTDKHTIKKNLLGALLFDSGETAEATRLK |
| | RTARRRYTRRKNRIRYLQEIFSEEMGKVDDSFFHRLEDSFYVEEDKKHDRHPIFANLVEEVAYHKN |
| | FPTIYHLRKKLVDNPDKVDLRLIYLALAHIVKFRGHFLIEGDFDIKNNDIQKIFNEFLQIYNSIFESNS |
| | LQKGNVQVEEILTDKISKSAKRERVLKLFPDEKSTGLFAEFLKLIVGNQADFKKHFDLEEKAPLQFS |
| | KDTYDEDLENLLGQIGDDFADLFLVAKKLYDAILLAGILSVDAPGTKAPLSASMIQRFDEHQEDLV |
| | RLKAFIRRSLPETYNEVFKDDTKNGYAGYIDGKTNQEDFYAYLKKLLTGLEGADYFLEKINREDLL |
| | RKQRTFDNGSIPYQIHLQEMRAILDKQAKFYPFLAKNKERIEKILTFRIPYYVGPLARGNSDFAWSIR |
| | KRNEKITPWNFEDVIDKESSAEAFINRMTSFDLYLPEEKVLPKHSLLYETFTVYNELTKVKFIAEGL |
| | RDYQFLDAEQKKEIVTLLFKEKRKVTVKDIEHYLHAIDGYDGIELKGIEKQFNSSLSTYHDLLKIIKD |
| | KEFMDDAKNEAILENIVHTLTIFEDREMIKQRLAQYASIFDEKVIKALTRRHYTGWGKLSAKLINGI |
| | HDKKSGKTILDYLIDDGYTNRNFMQLIHDDGLSFKEIIQKAQVVGKTDNVKQVVQELPGSPAIKKG |
| | ILQSIKIVDELVKVMGHAPESIVIEMARENQTTARGKKNSQQRYKRIEDSLKNLASGLDSQILKEHP |
| | TDNIQLQNDRLFLYYLQNGRDMYVGQELDINRLSDYDVDHIIPQAFIKDDSIDNRVLTSSKDNRGK |
| | SDNVPSEDVVKKMKNYWRQLLNAKLITQRKFDNLTKAERGGLSELDKAGFIKRQLVETRQITKHV |
| | AQILDSRMNAKYDENDKLIRDVKIITLKSKLVSDFRKDFQFYKVREINNYHHAHDAYLNAVVGTA |
| | LIKKYPKLESEFVYGDYKVYDVRKMIAKSEQEIGKATAKRFFYSNIMNFFKTEITLANGEIRKRPLIE |
| | TNEETGEIVWDKGRDFATVRKVLCMPQVNIVKKTEVQTGGFSKESILPKRNSDKLIARKKDWDPK |
| | KYGGFDSPTVAYSVLVVAKVEKGKSKKLKSVKELLGITIMERSSFEKNPIDFLEAKGYKEVKKDLII |
| | KLPKYSLFELENGRKRMLASAGELQKGNELALPSKYVNFLYLASHYEKLKGSPEDNEQKQLFVEQ |
| | HKHYLDEIIEQISEFSKRVILADANLDKVLSAYNKHRDKPIREQAENIIHLFTLTNLGAPAAFKYFDT |
| | TIDRKRYTSTKEVLDATLIHQSITGLYETRIDLSQLGGD |
| 15 | MTKPYSIGLDIGTNSVGWSVITDDYKVPAKKMKVLGNTDKQTIKKNLLGALLFDSGNTAEATRLK |
| | RTARRRYTRRRNRILYLQEIFAEEINKIDDSFFQRLDDSFLIVEDKQGSKHPIFGTLQEEKEYHKQFPT |
| | IYHLRKQLADSSQKADIRLIYLALAHIIKYRGHELFEGELKSENKDVQHLFNDFVEMEDKTVEGSYL |
| | SENLPNVADVLVEKISKSRRLENILQYFPNEKKNGLFGNFLALALGLTPNFKSNFDLAEDAKLQLSK |
| | DSYDEDLESLLAQIGDDYSDVFLKAKKLYDAILLSGILTVTDNETEAPLSSAMIMRYKEHEEDLALL |
| | KAYIRNISLDTYNEVFKDDTKNGYAGYIDGKTNQEDFYVYLKKLLAKLEGADYFLEKINREDLLR |
| | KQRTFDNGSIPYQIHLGEMRAILDKQAKFYPFLAENQDRIEKILTFRIPYYVGPLARGNSDFAWSIRK |
| | RDEKITPWNFEDVIDKESSAEAFINRMTSFDLYLPEEKVLPKHSLLYETFTVYNELTKVKFIAEGLR |
| | DYQFLDSEQKKQIVNQLFKEKRKVTEKDIEHYLHTVDGYDGIELKGIEKQFNSSLSTYHDLLKIIKD |
| | KEFMDDAKNEAILENIVHTLTIFEDREMIKQRLAQYDSIFDEKVIKALTRRHYTGWGKLSAKLINGI |
| | CDKQTGKTILDYLIDDGEMNRNFMQLINDDGLSFKEIIQKAQVVGKTDNVKQVVQELPGSPAIKKG |
| | ILQSIKIVDELVKVMGHAPESIVIEMARENQTTARGKKNSQQRYKRIEDSLKNLASGLDSNILKENP |
| | TDNIQLQNDRLFLYYLQNGRDMYTGKPLDINQLSSYDIDHIVPQAFIKDDSIDNRVLTSSKDNRGKS |
| | DNVPSEEVVKKMKAFWRQLLDAKLISQRKFDNLTKAERGGLTNDDKARFIQRQLVETRQITKHVA |
| | RILDERENTETDENNQKIRTVKIVTLKSNLVSNFRKEYQLYKVREINNYHHAHDAYLNAVVAKAIL |
| | TKYPQLEPEFVYGDYPKYNSYKTRKSATEKLFFYSNIMNFFKTEILLANGEIRKRPQIETNDETGEIV |
| | WNKCRDFATVRKVLSMPQVNIVKKTEVQTGGFSKESILPKRNSDKLIARKKDWDPKKYGGFDSPT |
| | VAYSVLVVAKVEKGKSKKLKSVKELLGITIMERSSFEKNPIDFLEAKGYKEVKKDLIIKLPKYSLFE |
| | LENGRKRMLASAGELQKGNELALPSKYVNFLYLASHYEKLKGSPEDNEQKQLFVEQHKHYLDEII |
| | EQISEFSKRVILADANLDKVLSAYNKHRDKPIREQAENIIHLFTLTNLGAPAAFKYFDTTIDRKRYTS |
| | TKEVLDATLIHQSITGLYETRIDLSQLGGD |
| 16 | MKKPYSIGLDIGTNSVGWAVITDSYKVPSKKMKVLGNTSKKYVKKNLLGALLFESGITAEGRRLK |
| | RTARRRYTRRRNRILYLQEIFAEEMNKIDESFFHRLDDSFLVPEDKRGSKYPIFATLEEEKEYHKQFP |
| | TIYHLRKHLADSKEKTDLRLIYLALAHMIKYRGHFLYEESFDIKNNDIQKIFNEFISIYDNTFEGSSLS |
| | GQNAQVEAIFTDKISKSAKRERVLKLFPDEKSTGLFSEFLKLIVGNQADFKKHFDLEEKAPLQFSKD |
| | TYDEDLENLLGQIGDDFADLFLVAKKLYDAILLSGILTVTDPSTKAPLSASMIERYENHQKDLAALK |
| | QFIQNNLQEKYDEVFSDQSKDGYAGYINGKTTQEAFYKYIKNLLNKVEGADYFLDKIEREDFLRKQ |
| | RTFDNGSIPHQIHLQEMHAILQRQGEHYPFLKDNKEKIEKILTFRIPYYVGPLARGNRDFAWLTRNS |
| | DQAIRPWNFEEVVDQASSAEDFINKMTNYDLYLPEEKVLPKHSLLYETFAVYNELTKVKFIAEGLR |
| | DYQFLDSGQKKQIVNQLFKEKRKVTEKDIIHYLHNVDGYDGIELKGIEKQFNASLATYHDLLKIIKD |
| | KEFMDDPKNEEILEKIIHTLTLFEDREMIKQRLEQYADIFDKSVLKKLSRRHYTGWGKLSAKLINGI |
| | RDKKTGKTILDYLIDDGYSNRNFMQLINDDSLSFKEKIKEAQRSGEGDDLREEVQNLAGSPAIKKGI |
| | LQSLKIVDELIEVMGRDPEHIVVEMARENQFTNQGRRNSQQRYKKLKNAIKNLDNNLNSKILKEYP |
| | TNNQALQNDRLFLYYIQNGKDMYTGEELDIDNLSQYDIDHIIPQAFIKDDSIDNRVLTSSAKNRGKS |
| | DDVPSIEIVKDRRNFWKQLLDSQLISQRKFDNLTKAERGGLTEADKAGFIKRQLVETRQITKHVAQI |
| | LDARFNTKRDENDKVIRDVKVITLKSNLVSQFRKDFQFYKVREINDYHHAHDAYLNAVVGTALIK |
| | KYPKLESEFVYGDYKVYDVRRMIAKSEQEIGKATAKRFFYSNILNFFKTEIKLANGEIRKRPLIETNE |
| | ETGEIVWNKEKDFATVRKVLSCPQVNIVKKTEVQTGGFSKESILPKRNSDKLIARKKDWDPKKYG |
| | GFDSPTVAYSVLVVAKVEKGKSKKLKSVKELLGITIMERSSFEKNPIDFLEAKGYKEVKKDLIIKLP |
| | KYSLFELENGRKRMLASAGELQKGNELALPSKYVNFLYLASHYEKLKGSPEDNEQKQLFVEQHKH |
| | YLDEIIEQISEFSKRVILADANLDKVLSAYNKHRDKPIREQAENIIHLFTLTNLGAPAAFKYFDTTIDR |
| | KRYTSTKEVLDATLIHQSITGLYETRIDLSQLGGD |
| 17 | MKKPYSIGLDIGTNSVGWAVITDDYKVPAKKMKVLGNTNKETIKKNLLGALLFDSGETAEATRLK |
| | RTARRRYTRRKNRLRYLQEIFSEEMAKVDDSFFHRLEESFLKDDDKQFDKHPIFGNKAEEKAYHK |
| | KFPTIYHLRKHLADSKEKADLRLVYLALAHMIKFRGHFLIEGELKAENTDIQALFKDFVEVYDKTID |
| | ESHISEIEVDAISILTEKISKSRRLEKLIQHYSLEKKNTLFGNLIALSLGLQPNFKTNFQLSEDAKLQFS |
| | KDTYEEDLEGLLGQVGDEYADLFASAKNLYDAILLSGILTVDDNSTKAPLSASMIKRYKEHKEDLA |
| | LFKQFIKSNIPEKYNHIFALGEDEKYNGYTGGNDKKLGHRSGKLVTEEEFYKYLKNILNKVEGADY |
| | FLDKIDREDFLRKQRTFDNGSIPHQIHLQEMHAILHRQGEHYPFLKENQDKIEKILTFRIPYYVGPLA |
| | RKGSRFAWAEYKTDEKITPWNFEDIIDKEKSAEKFITRMTLNDLYLPEEKVLPKHSLLYEEFAVYNE |
| | LTKVRYVTEQGKSFFFDANMKQEIFDGVFKVYRKVTKEKLLDFLDNEFDEFRIVDLTGLDKENKA |
| | FNASLGTYHDLKKIIDKDLLDNSSNKTILEDLVLTLTLFEDKKMIKKCLQKYDNLFDDKVLKKLKR |
| | RHYTGWGRLSAKLINGIRDKKSGKTILDELTDDGYVNRNFIQLINDGRLTFKKAIEKAQVSGQGDS |
| | LTEEVQSLAGSPAIKKGILQSLKIVDELIKVMGYKPENIVIEMARENQTTNQGRRKSQQRLKGLIDSI |
| | KEFGSNILKEHPTDNSALQNDRLYLYYLQNGKDMYTGDELDIDQLSNYDIDHIIPQAFLKDNSIDNK |
| | VLVSSASNRGKSDDVPSLEVVKKRKTFWHQLLKSKLISQRKFDNLTKAERGGLSPEDKAGFIQRQL |
| | VETRQITKHVARLLDERFNNKKDEDNKTLRTVKIITLKSSLVSQFRKDFQLYKVREINDFHHAHDA |
| | YLNAVVASALLKKYPKLEPEFVYGDYPKYNSYKTRKSATEKLFFYSNIMNFFKTEITLANGEIRKRP |
| | LIETNEETGEIVWNKCRDFATVRKVLSMPQVNIVKKTEVQTGGFSKESILPKRNSDKLIARKKDWD |
| | PKKYGGFDSPTVAYSVLVVAKVEKGKSKKLKSVKELLGITIMERSSFEKNPIDFLEAKGYKEVKKD |
| | LIIKLPKYSLFELENGRKRMLASAGELQKGNELALPSKYVNFLYLASHYEKLKGSPEDNEQKQLFV |
| | EQHKHYLDEIIEQISEFSKRVILADANLDKVLSAYNKHRDKPIREQAENIIHLFTLTNLGAPAAFKYF |
| | DTTIDRKRYTSTKEVLDATLIHQSITGLYETRIDLSQLGGD |
| 18 | MKKPYSIGLDIGTNSVGWAVVTDDYKVPAKKMKVLGNTDKSHIKKNLLGALLFDSGNTAEDRRL |
| | KRTARRRYTRRRNRILYLQEIFAEEMNKVDESFFHRLDDSFLVPEDKRGSKYPIFATLEEEKEYHKK |
| | FPTIYHLRKHLADSKEKTDLRLIYLALAHMIKYRGHFLYEESFDIKNNDIQKIFNEFISIYDNTFEGSS |
| | LSGQNAQVEEIFTDKISKSAKRERVLKLFPDEKSTGLFSEFLKLIVGNQADFKKHFDLEEKAPLQFS |
| | KDTYDEDLENLLGQIGDDFADLFLVAKKLYDAILLSGILTVTDPSTKAPLSASMIERYENHQKDLA |
| | ALKQFIKNNLSEKYDEVFSDQSKDGYAGYIEGKTTQEAFYKYIKKAIEKIEGSDYFLDKIERENFLR |
| | KQRTFDNGSIPHQIHLQEMHAIIHRQGEYYPFLKENKEKIEKILTFRIPYYVGPLARGKSDFAWLTRK |
| | SDDAIRPWNFEEMVDKESSAEAFIHRMTNYDLYLPEEKVLPKHSLLYEKFAVYNELTKVRYVTEQ |
| | GKSFFFDANMKQEIFDGIFKKYRKVTKEKLMDFLGKEFDEFRIVDLMGLDKDNKSFNASLGTYHD |
| | LKKIVSKDFLDNPENEDILENVVLTLTLFEDREMIRKRLEKYKDVLTEEQRKKLERRHYTGWGRLS |
| | AKLINGIRDKVTRKTILDYLIDDGTSNRNFMQLIHDDTLSFVDEIRLAQGSGEAEDYREEVQNLAGS |
| | PAIKKGILQSLKIVDELVGVMGYDPEHIVVEMARENQFTNQGRRNSQQRYKKIKDAIKNLDSNLNS |
| | KILKEYPTDNQALQNDRLFLYYLQNGKDMYTGEALDINQLSNYDIDHIIPQAFIKDDSLDNRVLTSS |
| | KDNRGKSDNVPSIEVVEKMKAFWQQLLDSKLISERKYNNLTKAERERGGLNELDKVGFIKRQLVE |
| | TRQITKHVAQILDARFNTEVDEKGKRIRKVKIITLKSNLVSNFRKEFGLYKVREINDYHHAHDAYL |
| | NAVVAKAILKKYPKLEPEFVYGDYQKYDIKRYISRSKDPKDIEKATEKYFFYSNLLNFFKEEVHYA |
| | DGTIVKRENIEYSKDTGEIAWNKEKDFATVRKVLSCPQVNIVKKTEVQTGGFSKESILPKRNSDKLI |
| | ARKKDWDPKKYGGFDSPTVAYSVLVVAKVEKGKSKKLKSVKELLGITIMERSSFEKNPIDFLEAK |
| | GYKEVKKDLIIKLPKYSLFELENGRKRMLASAGELQKGNELALPSKYVNFLYLASHYEKLKGSPED |
| | NEQKQLFVEQHKHYLDEIIEQISEFSKRVILADANLDKVLSAYNKHRDKPIREQAENIIHLFTLTNLG |
| | APAAFKYFDTTIDRKRYTSTKEVLDATLIHQSITGLYETRIDLSQLGGD |
| 19 | MKKSYSIGLDIGTNSVGWSVITDDYKVPAKKMKVLGNTDKKYIKKNLLGALLFDSGETAEATRLK |
| | RTARRRYTRRKNRLRYLQEIFAKEMAKVDESFFQRLEESFLTDDDKNFDSHPIFGNKAEEDAYHDE |
| | FPTIYHLRQYLADSPQKADLRLVYLALAHMIKFRGHFLIEGQLNAENTDVQTLFNDFVEVYDKTVE |
| | ESHLSEITVDALSILTEKVSKSRRLENLVKCYPTEKKNTLFGNLIALSLGLQPNFKTNFKLSEDAKLQ |
| | FSKDTYEEDLEELLGKIGDEYADLFASAKNLYDAILLSGILTVDDNTTKAPLSASMVKRYAEHHED |
| | LEKLKEFVKANKPELYHDIFKDETKNGYAGYIENGVKQDEFYKYLKNTLSKIAGSDYFLDKIERED |
| | FLRKQRTFDNGSIPHQIHLQEMHAILRRQGEYYPFLKENQAKIEKILTFRIPYYVGPLARKNSRFAW |
| | AEYHSDEKITPWNFEEIVDKEESATAFIHRMTNNDLYLPEEKVLPKHSLIYEEFTVYNELTKVKYVT |
| | DQGKAYYFDANMKQEIFDGVFKEHRKVTKDKLMNFLEKEFDEFRIVDLTGLDKENKSFNASLGTY |
| | HDLKKILDKSFLDDKTNETIIEDIIQTLTLFEDRDMIRQRLQKYSDIFTPQQLKKLERRHYTGWGRLS |
| | YKLINGIRNKENGKTILDYLIDDGYANRNFMQLINDDTLPFKQIIQEAQIIGDIDDIETVVHDLPGSPA |
| | IKKGILQSVKIVDELVKVMGHNPDNIVIEMARENQTTNRGRSQSQQRLKKLQESLKELGSNILNEEK |
| | PSYVEDKVENSHLQNDKLFLYYIQNGKDMYTGEELDIDHLSDYDIDHIIPQAFIKDNSIDNRVLTSS |
| | AKNRGKSDDVPSLDIVHDRKADWIRLYKSGLISKRKFDNLTKAEHGGLTEDDKAGFIKRQLVETR |
| | QITKHVAQILDSRENTERDENDKVIRDVKVITLKSNLVSQFRKDFELHKVREINDYHHAHDAYLNA |
| | VIGNALLVKYPQLEPEFVYGKFKHFHGHKENKATAKKFFYSNIMALFKKDVEYSDDTGEMVWNK |
| | EKDFATVRKVLSCPQVNIVKKTEVQTGGFSKESILPKGNSDKLIPRKTKKFYWDTKKYGGFDSPIV |
| | AYSVLVIADIEKGKAKKLKTVKALVGVTIMEKMTFERDPVAFLERKGYRNVQEENIIKLPKYSLFK |
| | LENGRKRLLASARELQKGNEIVLPNHLGTLLYHAKNIHKVDEPKHLDYVDKHKDEFKELLDVVSN |
| | FSKKYTLAEGNLEKIKELYAQNNGEDLKELASSFINLLTFTAIGAPATFKFFDKNIDRKRYTSTTEIL |
| | NATLIHQSITGLYETRIDLSKLGGD |
| 20 | MGKPYSIGLDIGTNSVGWAVITDDYKVPAKKMKVLGNTDKKYIKKNLLGALLFDSGETAEATRLK |
| | RTARRRYTRRKNRLRYLQEIFAEEMAKVDSTFFRRLNESFLTDDDKQFNACPIFGNLAEEDAYHEK |
| | FPTIYHLRKYLADNPDKADLRLIYLALAHMIKFRGHFLIEGELNSENTDIQKVFEEFVEIFERDVKSE |
| | LSESKLEIASVLLEKTAKARRLESVLAQFPNEKRTGLFAEFLKLIVGNQADFKKHFDLEEKAPLQFS |
| | KDTYDEDLENLLGQIGDGFAELFLVAKKLYDAILLSGILTVTDPSTKAPLSASMIERYESHQKDLAA |
| | LKQFVRRNLPEKYDEVFSDQSKDGYAGYIDGKTTQEAFYKYIKKLIEKIDGKGYFLAKIKQEDLLR |
| | KQRTFDNGSIPHQIHLGELHAIIRRQEDFYPFLKENRKKIEKLLTFRIPYYVGPLARGNNDFAWLTRN |
| | SDQAIRPWNFEEIVDKASSAEDFIHKMTNYDLYLPEEKVLPKHSLLYETFTVYNELTKVKFIAEGLR |
| | DYQFLDSGQKKQIVNQLFKEKRKVTEKDIIHYLHTVDGYDGIELKGIEKQFNSSLSTYHDLLKIIKD |
| | KEFMDDAKNEVILENIVHTLTIFEDREMIKQRLAQYSSIFDEKVIKALTRRHYTGWGKLSAKLINGI |
| | CDKKTGKTILDYLIDDGYTNRNFMQLINDDSLSFKEIIEKAQVSGKGDSVHEQIANLAGSPAIKKGIL |
| | QSIKIVDELVKVMGHKPENIVIEMARENQTTAKGKKNSQQRYKRIEEGLENLAKELGSQILKEHPA |
| | DNIQLQNDRLYLYYLQNGKDMYTGEALDINRLSDYDIDHIVPQAFIKDNSIDNKVLTYSKENRGKS |
| | DNVPSEDVVKKMKNYWRQLLNAKLITQRKFDNLTKAERGGLSELDKAGFIKRQLVETRQITKHVA |
| | QILDSRMNAKYDENDKLIRDVKIITLKSKLVSDFRKDFQFYKVREINNYHHAHDAYLNAVVGTALI |
| | KKYPKLESEFVYGDYKVYDVRKMIAKSEQEIGKATAKQFFYSNIMNFFKTEITLANGEIRKRPVIET |
| | NEETGEIVWDKGRDFATVRKVLCMPQVNIVKKTEVQTGGFSKESILPKRNSDKLIARKKDWDPKK |
| | YGGFDSPTVAYSVLVVAKVEKGKSKKLKSVKELLGITIMERSSFEKNPIDFLEAKGYKEVKKDLIIK |
| | LPKYSLFELENGRKRMLASAGELQKGNELALPSKYVNFLYLASHYEKLKGSPEDNEQKQLFVEQH |
| | KHYLDEIIEQISEFSKRVILADANLDKVLSAYNKHRDKPIREQAENIIHLFTLTNLGAPAAFKYFDTTI |
| | DRKRYTSTKEVLDATLIHQSITGLYETRIDLSQLGGD |
| 21 | MKKSYSIGLDIGTNSVGWSVVTDDYKVPAKKMKVFGNTDKKYIKKNLLGALLFDSGETAEGTRL |
| | KRTARRRYTRRRNRILYLQEIFAKEMAKVDESFFQRLEESFLTDDDKNFDSHPIFGNKAEEDAYHE |
| | QFPTIYHLRKHLADSSEKADLRLVYLALAHMIKFRGHFLIEGQLKAENTNVQTLFNDFVEVYDKTV |
| | EESHLAEITVDALSILTEKVSKSRRLENLLKYYPTEKKNTLFGNLIALSLGLQPNFKTNFKLSEDAKL |
| | QFSKDTYEEDLEELLGKIGDEYADLFTSAKNLYDAILLSGILTVDDKSTKAPLSASMLKRYVEHHE |
| | DLEKLKDFVKANKPELYHDIFKDETKNGYAGYIENGVKQDEFYKYLKNTLSKIAGSDYFLDKIERE |
| | DFLRKQRTFDNGSIPHQVHLQEMRAILRRQGEYYPFLKENQAKIEKILTFRIPYYVGPLARKDSRFA |
| | WAEYHSDEKITPWNFDKVIDKESSARAFIEHMTNNDLYLPEEKVLPKHSPLYEKYTVYNELTKVK |
| | YVTEQGKSSFFDANMKQEIFEHVFKKNRKVTKDKFLNYLNKEFPEYRIHDLIGLDKENKSFNASLG |
| | TYHDLKKILDKSFLDNKENEEIIEDVVLTLTLFEDRDMIRQRLQKYSDIFTKDQLKKLERRHYTGW |
| | GRLSYKLINGIRDKQSNKTILDYLLEDDGVKKNINRNFMQLINDDSLSFKTIIQEAQVIGDIDDIKAV |
| | VHDLPGSPAIKKGILQSVKIVDELVKVMGHNPDNIVIEMARENQTTNRGRSQSQQRLKKLQESLKE |
| | LGSNILNEEKPSYIEDKVENSHLQNDRLFLYYIQNGKDMYTGDELDIDHLSDYDIDHIIPQAFIKDNS |
| | IDNRVLTSSAKNRGKSDDVPSLDIVRERKADWIRLYKAGLISKRKFDNLTKAERGGLTENDKAGFI |
| | KRQLVETRQITKHVAQILDARFNTKRDENDKVIRDVKIITLKSNLVSQFRKEFGFYKVREINDYHHA |
| | HDAYLNAVVGTALIKKYPKLESEFVYGDYKVYDVRKMIAKSEQEIGKATAKRFFYSNIMNFFKTEI |
| | KLANGEIRKRPLIETNEETGEVVWNKERDFAIVRKVLSCPQVNIVKKTEVQTGGFSKESILPKGNSD |
| | KLIARKKDWDPKKYGGFDSPIVAYSVLVVAKVAKGKSQKIKTIKELVGIKIMEQDEFEKDPIAFLEK |
| | KGYQDIQKDLIIKLPKYSLFELENGRKRLLASAGELQKGNELALPSKYVNFLYLASHYEKLKGSPE |
| | DNEQKQLFVEQHQHYLDEIIEQISEFSKRVILADANLDKVLSAYNKHRDKPIREQAENIIHLFTLTNL |
| | GAPAAFKYFDTTIDRKRYTSTKEVLDATLIHQSITGLYETRIDLSQLGGD |
| 22 | MKKSYSIGLDIGTNSVGWSVVTDDYKVPAKKMKVFGNTDKKYIKKNLLGALLFDSGETAEGTRL |
| | KRTARRRYTRRRNRILYLQEIFAKEMAKVDESFFQRLEESFLTDDDKNFDSHPIFGNKAEEDAYHE |
| | QFPTIYHLRKHLADSSEKADLRLVYLALAHMIKFRGHFLIEGQLKAENTNVQTLFNDFVEVYDKTV |
| | EESHLAEITVDALSILTEKVSKSRRLENLLKYYPTEKKNTLFGNLIALSLGLQPNFKTNFKLSEDAKL |
| | QFSKDTYEEDLEELLGKIGDEYADLFTSAKNLYDAILLSGILTVDDKSTKAPLSASMLKRYVEHHE |
| | DLEKLKDFVKANKPELYHDIFKDETKNGYAGYIENGVKQDEFYKYLKNTLSKIAGSDYFLDKIERE |
| | DFLRKQRTFDNGSIPHQVHLQEMRAILRRQGEYYPFLKENQAKIEKILTFRIPYYVGPLARKDSRFA |
| | WAEYHSDEKITPWNFDKVIDKESSARAFIEHMTNNDLYLPEEKVLPKHSPLYEKYTVYNELTKVK |
| | YVTEQGKSSFFDANMKQEIFEHVFKKNRKVTKDKFLNYLNKEFPEYRIHDLIGLDKENKSFNASLG |
| | TYHDLKKILDKSFLDNKENEEIIEDVVLTLTLFEDRDMIRQRLQKYSDIFTKDQLKKLERRHYTGW |
| | GRLSYKLINGIRDKQSNKTILDYLLEDDGVKKNINRNFMQLINDDSLSFKTIIQEAQVIGDIDDIKAV |
| | VHDLPGSPAIKKGILQSVKIVDELVKVMGHNPDNIVIEMARENQTTNRGRSQSQQRLKKLQESLKE |
| | LGSNILNEEKPSYIEDKVENSHLQNDRLFLYYIQNGKDMYTGDELDIDHLSDYDIDHIIPQAFIKDNS |
| | IDNRVLTSSAKNRGKSDDVPSLDIVRERKADWIRLYKAGLISKRKFDNLTKAERGGLTENDKAGFI |
| | KRQLVETRQITKHVAQILDARFNTKRDENDKVIRDVKIITLKSNLVSQFRKEFGFYKVREINDYHHA |
| | HDAYLNAVVGTALIKKYPKLESEFVYGDYKVYDVRKMIAKSEQEIGKATAKRFFYSNIMNFFKTEI |
| | KLANGEIRKRPLIETNEETGEVVWNKERDFAIVRKVLSCPQVNIVKKTEVQTGALTNESIYARGSFD |
| | KLISRKHRFESSKYGGFGSPTVTYSVLVVAKSKVQDGKVKKIKTGKELIGITLLDKLVFEKNPLKFIE |
| | DKGYGNVQIDKCIKLPKYSLFEFENGTRRMLASVMANNNSRGDLQKANEMFLPAKLVTLLYHAH |
| | KIESSKELEHEAYILDHYNDLYQLLSYIERFASLYVDVEKNISKVKELFSNIESYSISEICSSVINLLTL |
| | TASGAPADFKFLGTTIPRKRYGSPQSILSSTLIHQSITGLYETRIDLSQLGGD |
| 23 | MTKPYSIGLDIGTNSVGWAVITDSYKVPSKKMKVLGNTSKKYVKKNLLGALLFESGITAEGRRLK |
| | RTARRRYTRRRNRILYLQEIFAAEMNKIDESFFQRLDDSFLVPDDKRDSKYPIFGNLAEEKAYHEKF |
| | PTIYHLRKYLADSSEKADLRLVYLALAHMIKYRGHFLIEGDFNSRNNDIKKNFQDFLDSYNAIFESD |
| | LSLENSKQLEEIVKDKISKSAKRERVLKLFPDEKSTGLFSEFLKLIVGNQAEFKKHFDLEEKAPLQFS |
| | KDTYDEDLENLLGQIGDDFADLFLVAKKLYDAILLSGILTVTDPSTKAPLSASMIERYENHQKDLA |
| | ALKQFIQNNLQEKYDEVFSDQSKDGYAGYIDGKTTQEAFYKYIKNILSKLEGSDYFLDKIEREDFLR |
| | KQRTFDNGSIPHQIHLQEMNAILRRQGEHYPFLKENKEKIQQILTFRIPYYVGPLARGNRDFAWLTR |
| | NSDQAIRPWNFEEIVDQASSAEEFINKMTNYDLYLPEEKVLPKHSLLYETFAVYNELTKVKFIAEGL |
| | RDYQFLDSGQKKQIVNQLFKEKRKVTEKDIIHYLHNVDGYDGIELKGIEKQFNASLSTYHDLLKIIK |
| | DKEFMDDPKNEEILENIVHTLTIFEDREMIKQRLAQYDSLFDEKVIKALARRHYTGWGRLSAKLID |
| | GIYDKQSRKTILDYLIDDGEINRNFMQLINDDGLSFKEIIQKAQVVGKTDDVKQVVQELPGSPAIKK |
| | GILQSIKIVDELVKVMGHAPESIVIEMARENQTTARGKKNSQQRYKRIEDALKNLAPGLDSNILKEN |
| | PTDNIQLQNDRLFLYYLQNGKDMYTDEELDINQLSNYDIDHIIPQAFIKDDSLDNRVLTSSKDNRGK |
| | SDNVPSLEVVQKRKAFWQQLLDSKLISERKENNLTKAERGGLDERDKVGFIKRQLVETRQITKHVA |
| | QILDARFNTEVDENNQRIRKVKIVTLKSNLVSNFRKEFGLYKVREINDYHHAHDAYLNAVVAKAIL |
| | KKYPKLEPEFVYGDYQKYDVKRYISRSKDPKEIEKATQKYFFYSNLLNFFKEEVHYADGTIIKRENI |
| | EYSKDTGEIAWNKEKDFATVRKVLSCPQVNIVKKTEVQTGGFSKESILPKRNSDKLIARKKDWDPK |
| | KYGGFDSPTVAYSVLVVAKVEKGKSKKLKSVKELLGITIMERSSFEKNPIDFLEAKGYKEVRKDLI |
| | VKLPKYSLFELENGRKRMLASAGELQKGNELALPSKYVNFLYLASHYEKLKGSPEDNEQKQLFVE |
| | QHKHYLDEIIEQISEFSKRVILADANLDKVLSAYNKHRDKPIREQAENIIHLFTLTNLGAPAAFKYFD |
| | TTIDRKRYTSTKEVLDATFIHQSITGLYETRIDLSQLGGD |
| 24 | MTKPYSIGLDIGTNSVGWAVITDSYKVPSKKMKVLGNTSKKYVKKNLLGALLFESGITAEGRRLK |
| | RTARRRYTRRRNRILYLQEIFSTEMAKIDESFFQRLDDSFLVPDDKRDSKYPIFGNLVEEKVYHDEF |
| | PTIYHLRKYLADSSKKADLRLVYLALAHMIKYRGHFLIEGDFNSRNNDIKKNFQNFLDSYNAIFESD |
| | LSLENSKQLEEIVKDKISKSAKRERVLKLFPDEKSTGLFSEFLKLIVGNQADFKKHFDLEEKAPLQFS |
| | KDTYDEDLENLLGQIGDDFADLFLVAKKLYDAILLSGILTVTDPSTKAPLSASMIERYENHQKDLA |
| | ALKQFIQNNLQEKYDEVFSDQSKDGYAGYIDGKTTQEAFYKYIKNLLSKLEGADYFLDKIEREDFL |
| | RKQRTFDNGSIPHQIHLQEMNAILRRQGEHYPFLKENKEKIQQILTFRIPYYVGPLARGNRDFAWLT |
| | RNSDQAIRPWNFEEIVDKASSAEDFINKMTNYDLYLPEEKVLPKHSLLYETFAVYNELTKVKFIAEG |
| | LRDYQFLDSGQKKQIVNQLFKEKRKVTEKDIIQFLHNVDGYDGIELKGIEKQFNASLSTYHDLLKII |
| | KDKEFMDDAKNEEILENIVHTLTIFEDREMIKQRLAQYDSLFDEKVIKALARRHYTGWGKLSAKLI |
| | NGIRDKQSQKTILDYLIDDGEINRNFMQLIHDDGLSFKDIIQKAQVVGDTDDLKQVVQNLPGSPAIK |
| | KGILQSIKIVDELVKVMGGDPESIVVEMARENQFTNQGKRNSQQRYKRIEDAIKNLDSNLNSKILKE |
| | YPTDNQALQNDRLFLYYLQNGKDMYTGEALDINQLSNYDIDHIIPQAFIKDDSLDNRVLTSSKDNR |
| | GKSDNVPSIEVVKARKAFWQQLLDSKLISERKFNNLTKAERGGLNELDKVGFIKRQLVETRQITKH |
| | VAQILDARFNTEVNEENQKIRTVKIITLKSNLVSNFRKEFGLYKVREINDYHHAHDAYLNAVVAKA |
| | ILKKYPKLEPEFVYGDYKRYDLQKLRAKSEKEMGKATAKKFFYSNLMNFFKKEVRLADGTVIVRP |
| | VIEYSKDTGEIAWNKEKDFATVRKVLSCPQVNIVKKTEVQTGGFSKESILPKRNSDKLIARKKDWD |
| | PKKYGGFDSPTVAYSVLVVAKVEKGKSKKLKSVKELLGITIMERSSFEKNPIDFLEAKGYKEVRKD |
| | LIVKLPKYSLFELENGRKRMLASAGELQKGNELALPSKYVNFLYLASHYEKLKGSPEDNEQKQLFV |
| | EQHKHYLDEIIEQISEFSKRVILADANLDKVLSAYNKHRDKPIREQAENIIHLFTLTNLGAPAAFKYF |
| | DTTIDRKRYTSTKEVLDATFIHQSITGLYETRIDLSQLGGD |
| 25 | MTKPYSIGLDIGTNSVGWAVITDNYKVPSKKMKVLGNTSKKYVKKNLLGALLFESGITAEGRRLK |
| | RTARRRYTRRRNRILYLQEIFSTEMAKIDESFFQRLDDSFLVPDDKRDSKYPIFGNLVEEKAYHDEF |
| | PTIYHLRKYLADSSKKADLRLVYLALAHMIKYRGHFLIEGDFNSRNNDIKKNFQNFLDSYNAIFESD |
| | LSLENSKQLEEIVKDKISKSAKRERVLKLFPDEKSTGLFSEFLKLIVGNQADFKKHFDLEEKAPLQFS |
| | KDTYDEDLENLLGQIGDDFADLFLVAKKLYDAILLSGILTVTDPSTKAPLSASMIERYENHQKDLA |
| | ALKQFIQNNLQEKYDEVFSDQSKDGYAGYIDGKTTQEAFYKYIKNLLSKLEGADYFLEKIEREDFL |
| | RKQRTFDNGSIPHQIHLQEMNAILHNQGEYYPFLKENSEKIQQILTFRIPYYVGPLARGNRDFAWLT |
| | RNSDQAIRPWNFEEIVDKASSAEDFINKMTNYDLYLPEEKVLPKHSLLYETFAVYNELTKVKFIAEG |
| | LRDYQFLDSGQKKQIVNQLFKEKRKVTEKDIIQFLHNVDGYDGIELKGIEKQFNASLSTYHDLLKII |
| | KDKEFMDDPKNEEILENIIHTLTIFEDREMIKQRLAQYDSIFDEKVIKALTRRHYTGWGKLSAKLIN |
| | GIRDKKSGKTILDYLIDDDKINRNFMQLIHDDGLSFKEIIQKAQVVGKTDDVKQVVQELPGSPAIKK |
| | GILQSIKIVDELVKVMGHAPESIVIEMARENQTTNQGKRNSQQRYKRIEEAIKNLAPELDGNILKEY |
| | PTDNQALQNDRLFLYYLQNGKDMYTGEELDINQLSNYDIDHIVPQAFIKDDSIDNRVLTSSKDNRG |
| | KSDNVPSLEVVQKRKAFWQQLLDSKLISERKENNLTKAERGGLDERDKVGFIRRQLVETRQITKHV |
| | AQILDARFNTEVNEKNQKIRSVKIITLKSNLVSNFRKEFELYKVREINDYHHAHDAYLNAVVAKAIL |
| | KKYPKLEPEFVYGDYQKYDIKRYISRSKDPKDIEKATEKYFFYSNLLNFFKEEVHYADGTIIKRENIE |
| | YSKDTGEIAWNKEKDFATVRKVLSCPQVNIVKKTEVQTGGFSKESILPKRNSDKLIARKKDWDPK |
| | KYGGFDSPTVAYSVLVVAKVEKGKSKKLKSVKELLGITIMERSSFEKNPIDFLEAKGYKEVRKDLI |
| | VKLPKYSLFELENGRKRMLASAGELQKGNELALPSKYVNFLYLASHYEKLKGSPEDNEQKQLFVE |
| | QHKHYLDEIIEQISEFSKRVILADANLDKVLSAYNKHRDKPIREQAENIIHLFTLTNLGAPAAFKYFD |
| | TTIDRKRYTSTKEVLDATFIHQSITGLYETRIDLSQLGGD |
| 26 | MTKPYSIGLDIGTNSVGWAVITDDYKVPSKKMKVLGNTSKKYVKKNLLGALLFESGITAEGRRLK |
| | RTARRRYTRRRNRILYLQEIFSTEMNKIDESFFQRLDDSFLVPDDKRDSKYPIFGNLAQEKTYHDEF |
| | PTIYHLRKYLADSSKKADLRLVYLALAHMIKYRGHFLIEGDFNSKSNSIKETFSKFLDSYNAIFESDL |
| | SLENSKQLEEIVKDKISKSAKRERVLKLFPDEKSTGLFSEFLKLIVGNQADFKKHFDLEEKAPLQFSK |
| | DTYDEDLENLLGQIGDGFADLFLVAKKLYDAILLSGILTVTDPSTKAPLSASMIERYENHQKDLATL |
| | KQFIKNNLSEKYDEVFSDQSKDGYAGYIDGKTTQEAFYKYIKNLLSKLEGADYFLDKIEREDFLRK |
| | QRTFDNGSIPHQIHLQEMNAILRRQGEHYPFLKENREKIEKILTFRIPYYVGPLARGNRDFAWLTRN |
| | SDQAIRPWNFEEIVDQASSAEDFINKMTNYDLYLPEEKVLPKHSLLYETFAVYNELTKVKFIAEGLR |
| | DYQFLDSGQKKQIVNQLFKEKRKVTEKDIIQFLHNVDGYDGIELKGIEKQFNASLSTYHDLLKIIKD |
| | KEFMDDPKNEEILENIVHTLTIFEDREMIKQRLAQYDSLFDEKVIKALTRRHYTGWGKLSAKLINGI |
| | RDKKSGKTILDYLIDDGEINRNFMQLIHDDGLSFKEIIQKAQVFGKTDDVKQVVQNLPGSPAIKKGI |
| | LQSIKIVDELVKVMGKDPESIVIEMARENQTTNQGRRNSQQRYKKIEEAIKKLDIDLNSKILKEYPT |
| | NNQALQNERLFLYYLQNGKDMYTDQELDINRLSDYDIDHIVPQAFIKDDSLDNRVLTSSKDNRGKS |
| | DNVPSEEVVQKMKGFWQQLLKSKLISKRKFDNLTKAERGGLTEKDKAGFIKRQLVETRQITKHVA |
| | QILDARFNTEVNEKNQKIRKVKIVTLKSNLVSNFRKEFGLYKVREINDYHHAHDAYLNAVVAKAIL |
| | KKYPKLEPEFVYGDYQKYDIKRYISRSKDPKDIEKATEKYFFYSNLLNFFKEEVHYADGTIIKRENIE |
| | YSKDTGEIAWNKEKDFATVRKVLSCPQVNIVKKTEVQTGGFSKESILPKRNSDKLIARKKDWDPK |
| | KYGGFDSPTVAYSVLVVAKVEKGKSKKLKSVKELLGITIMERSSFEKNPIDFLEAKGYKEVRKDLI |
| | VKLPKYSLFELENGRKRMLASAGELQKGNELALPSKYVNFLYLASHYEKLKGSPEDNEQKQLFVE |
| | QHKHYLDEIIEQISEFSKRVILADANLDKVLSAYNKHRDKPIREQAENIIHLFTLTNLGAPAAFKYFD |
| | TTIDRKRYTSTKEVLDATFIHQSITGLYETRIDLSQLGGD |
| 27 | MTKPYSIGLDIGTNSVGWAVITDSYKVPSKKMKVLGNTSKKYVKKNLLGALLFESGITAEGRRLK |
| | RTARRRYTRRRNRILYLQEIFSTEMSKIDESFFQRLDDSFLVPDDKRDSKYPIFGNLVKEKTYHDEFP |
| | TIYHLRKYLADSSKKADLRLVYLALAHMIKYRGHFLIEGDFNSRNNDIKKNFQNFLDSYNAIFESDL |
| | SLENSKQLEEIVKDKISKSAKRERVLKLFPDEKSTGLFSEFLKLIVGNQADFKKHFDLEEKAPLQFSK |
| | DTYDEDLENLLGQIGDGFADLFLVAKKLYDAILLSGILTVTDPSTKAPLSASMIERYENHQKDLAAL |
| | KQFIKTNLPEKYDEVFSDQSKDGYAGYIDGKTTQEAFYKYIKNTLSKIEGSDYFLDKIEREDFLRKQ |
| | RTFDNGSIPHQIHLQEMNAIIRRQGEHYPFLKENKEKIEKILTFRIPYYVGPLARGNRDFAWLTRNSD |
| | QAIRPWNFEEIVDQASSAEDFINKMTNYDLYLPEEKVLPKHSLLYETFAVYNELTKVKFIAEGLRD |
| | YQFLDSGQKKQIVNQLFKEKRKVTEKDIIQFLHNVDGYDGIELKGIEKQFNASLSTYHDLLKIIKDK |
| | EFMDDPKNQEILENIVHTLTIFEDREMIKQRLAQYDSIFDEKVIKALTRRHYTGWGKLSAKLINGIC |
| | DKKTGKTILDYLIDDGEINRNFMQLIHDDGLSFKEIIQKAQVFGKTDDVKQVVQELPGSPAIKKGIL |
| | QSIKIVDELVKVMGHAPESIVIEMARENQTTNQGRRNSQQRYKKIKEAIKNLDSNLNSKILKEYPTD |
| | NQALQNDRLFLYYLQNGKDMYTGEPLDINQLSSYDIDHIIPQAFIKDDSLDNRVLTSSKDNRGKSD |
| | NVPSLEIVEKMKGFWQQLLNSKLISERKFNNLTKAERGGLDERDKVGFIKRQLVETRQITKHVAQI |
| | LDARFNTEVNDKNQKIRTVKIITLKSNLVSNFRKEFGLYKVREINDYHHAHDAYLNAVVAKAILKK |
| | YPKLEPEFVYGDYQKYDLKRYISKSKNPKELDKATQKYFFYSNLLNFFKEEVHYADGTIIKRENIEY |
| | SKDTGEIAWNKEKDFATVRKVLSCPQVNIVKKTEVQTGGFSKESILPKRNSDKLIARKKDWDPKK |
| | YGGFDSPTVAYSVLVVAKVEKGKSKKLKSVKELLGITIMERSSFEKNPIDFLEAKGYKEVRKDLIV |
| | KLPKYSLFELENGRKRMLASAGELQKGNELALPSKYVNFLYLASHYEKLKGSPEDNEQKQLFVEQ |
| | HKHYLDEIIEQISEFSKRVILADANLDKVLSAYNKHRDKPIREQAENIIHLFTLTNLGAPAAFKYFDT |
| | TIDRKRYTSTKEVLDATFIHQSITGLYETRIDLSQLGGD |
| 28 | MTKPYSIGLDIGTNSVGWAVITDSYKVPSKKMKVLGNTSKKYVKKNLLGALLFESGITAEGRRLK |
| | RTARRRYTRRRNRILYLQEIFDTEMKEIDESFFQRLDDSFLVPDDKRDSKYPIFGNLAQEKTYHDEF |
| | PTIYHLRKYLADSSKKADLRLVYLALAHMIKYRGHFLIEGDFNSRNNDIKKNFQDFLDSYNAIFESD |
| | LSLENSKQLEEIVKDKISKSAKRERVLKLFPDEKSTGLFSEFLKLIVGNQADFKKHFDLEEKAPLQFS |
| | KDTYDEDLENLLGQIGDDFADLFLVAKKLYDAILLSGILTVTDPSTKAPLSASMIERYENHQKDLA |
| | ALKQFIKNNLSEKYDEVFSDESKDGYAGYINGKTTQEAFYKYIKNLLSKLEGADYFLDKIEREDFL |
| | RKQRTFDNGSIPHQIHLQEMNAIIRRQGEHYPFLQENKEKIEKILTFRIPYYVGPLARGNRDFAWLTR |
| | NSDQAIRPWNFEEIVDKASSAEDFINKMTNYDLYLPEEKVLPKHSLLYETFAVYNELTKVKFIAEGL |
| | RDYQFLDSGQKKQIVNQLFKEKRKVTEKDIIQFLHNVDGYDGIELKGIEKQFNASLSTYHDLLKIIK |
| | DKEFMDDPKNEEILENIVHTLTIFEDREMIKQRLAQYDSLFDEKVIKALTRRHYTGWGKLSAKLING |
| | IYDKQSQKTILDYLIDDDKINRNFMQLINDDGLSFKEIIQKAQVVGKTDDVKQVVQELPGSPAIKKG |
| | ILQSIKIVDELVKVMGHAPESIVIEMARENQTTARGRKNSQQRYKKIMDAIKNLDSNLNSKILKEYP |
| | TDNQALQNDRLFLYYLQNGKDMYTGEPLDINQLSSYDIDHIIPQAFIKDDSLDNRVLTSSKDNRGK |
| | SDNVPSIEVVKKMKGFWQQLLDSKLISERKENNLTKAERGGLDERDKVGFIRRQLVETRQITKHVA |
| | QILDARFNTEVDEKGKRIRKVKIITLKSNLVSNFRKEFGLYKVREINDYHHAHDAYLNAVVAKAIL |
| | KKYPKLEPEFVYGDYQKYDVKRYISRSKDPKEVEKATEKYFFYSNLLNFFKEEVHYADGTIVKRE |
| | NIEYSKDTGEIAWNKEKDFATVRKVLSCPQVNIVKKTEVQTGGFSKESILPKRNSDKLIARKKDWD |
| | PKKYGGFDSPTVAYSVLVVAKVEKGKSKKLKSVKELLGITIMERSSFEKNPIDFLEAKGYKEVRKD |
| | LIVKLPKYSLFELENGRKRMLASAGELQKGNELALPSKYVNFLYLASHYEKLKGSPEDNEQKQLFV |
| | EQHKHYLDEIIEQISEFSKRVILADANLDKVLSAYNKHRDKPIREQAENIIHLFTLTNLGAPAAFKYF |
| | DTTIDRKRYTSTKEVLDATFIHQSITGLYETRIDLSQLGGD |
| 29 | MKKPYSIGLDIGTNSVGWAVVDDDYKVPAKKMRVFGDTDRSHIKKNLLGTLLFDDGNTAESRRL |
| | KRTARRRYTRRRNRILYLQEIFAEEMNKVDESFFHRLDDSFLVPEDKRGSKYPIFATLEEEKEYHKK |
| | FPTIYHLRKHLADSKEKTDLRLIYLALAHMIKYRGHFLYEESFDIKNNDIQKIFNEFISLYDNTFEGSS |
| | LSGQNAQVEAIFTDKISKSAKRERVLKLFPDEKSTGLFSEFLKLIVGNQADFKKHFDLEEKAPLQFS |
| | KDTYDEDLENLLGQIGDDFADLFLVAKKLYDAILLSGILTVTDPSTKAPLSASMIERYENHEEDLAA |
| | LKQFVKANLPEKYDEVFSDQSKDGYAGYIDGKTTQEAFYKYIKNLLSKLEGADYFLDKIEREDFLR |
| | KQRTFDNGSIPHQIHLQEMNAILRRQGEHYPFLKDNKEKIEKILTFRIPYYVGPLARGNRDFAWLTR |
| | NSDQAIRPWNFEEVVDKASSAEDFINKMTNYDLYLPEEKVLPKHSLLYETFAVYNELTKVKFIAEG |
| | LRDYQFLDSGQKKQIVNQLFKTKRKVTEKDIINYLHKVDGYDGVEISGVEDRFNASLGTYHDLLKI |
| | IKDKSFLDDKKNEEIIEDIVLTLTLFEDRDMIRQRLQKYSDIFTPQQLKKLERRHYTGWGRLSYKLIN |
| | GIRNKENNKSILDYLIDDGYANRNFMQLINDDTLPFKQIIQKSQVVGDIDDIKTVVHDLPGSPAIKK |
| | GILQSVKIVDELVKVMGHNPDNIVIEMARENQTTNRGRSQSQQRLKKLQESLKELGSNILNVEKPS |
| | YIEDKVENNHLQNDKLFLYYIQNGKDMYTGEELDIDHLSDYDIDHIIPQAFIKDDSIDNRVLTSSAK |
| | NRGKSDDVPSLDIVRGRKAEWIRLYKAKLISQRKFDNLTKAERGGLTENDKAGFIKRQLVETRQIT |
| | KHVAQILDARFNTKRDENDKLIREVKVITLKSNLVSQFRKEFGFYKVREINDYHHAHDAYLNAVV |
| | GTAILKKYPKLTSEFVYGEYKKYDIRKFIKSDDHSEMGKATAKYFFYSNLLNFFKEEIKYANGKIVV |
| | RPVVEYSKDTGEIAWNKEKDFATVRKVLSCPQVNIVKKTEVQTGGFSKESILPKRNSDKLIARKKD |
| | WDPKKYGGFDSPTVAYSVLVVAKVEKGKSKKLKSVKELLGITIMERSSFEKNPIDFLEAKGYKEVR |
| | KDLIVKLPKYSLFELENGRKRMLASAGELQKGNELALPSKYVNFLYLASHYEKLKGSPEDNEQKQ |
| | LFVEQHKHYLDEIIEQISEFSKRVILADANLDKVLSAYNKHRDKPIREQAENIIHLFTLTNLGAPAAF |
| | KYFDTTIDRKRYTSTKEVLDATFIHQSITGLYETRIDLSQLGGD |
| 30 | MTKPYSIGLDIGTNSVGWAVITDSYKVPSKKMKVLGNTSKKYVKKNLLGALLFESGITAEGRRLK |
| | RTARRRYTRRRNRILYLQEIFSTEMAKIDESFFQRLDDSFLVPDDKRDSKYPIFGNLVEEKTYHDEFP |
| | TIYHLRKYLADSSKKADLRLVYLALAHMIKYRGHFLIEGDFNSRNNDIKKNFQNFLDSYNAIFESDL |
| | SLENSEQVEEIVKDKISKSAKRERVLKLFPDEKSTGLFSEFLKLIVGNQADFKKHFDLEEKAPLQFSK |
| | DTYDEDLENLLGQIGDDFADLFLVAKKLYDAILLSGILTVTDPSTKAPLSASMIERYENHQKDLATL |
| | KQFVKANLPEKYDEVFSDQSKDGYAGYIDGKTTQEAFYKYIKNLLSKFEGADYFLDKIEREDFLRK |
| | QRTFDNGSIPHQIHLQEMNAIIRRQGEHYPFLKENKEKIEKILTFRIPYYVGPLARGNRDFAWLTRNS |
| | DQAIRPWNFEEVVDQASSAEEFINKMTNYDLYLPEEKVLPKHSLLYETFAVYNELTKVKFIAEGLR |
| | DYQFLDSGQKKQIVNQLFKEKRKVTEKDIIHYLHTVDGYDGIELKGIEKQFNASLSTYHDLLKIIKD |
| | KEFMDDPKNEEILENIVHTLTIFEDREMIKQRLAQYDSLFDEKVIKALTRRHYTGWGKLSAKLINGI |
| | YDKQSQKTILDYLIDDDKINRNFMQLIHDDGLSFKEIIQKAQVFGKTDDVKQVVQELPGSPAIKKGI |
| | LQSIKIVDELVKVMGHAPESIVIEMARENQTTARGRKNSQQRYKKIMDAIKNLDSNLNSKILKEYPT |
| | DNQALQNDRLFLYYLQNGKDMYTGEPLDINQLSSYDIDHIIPQAFIKDDSLDNRVLTSSKDNRGKS |
| | DNVPSLEIVEKMKGFWQQLLDSKLISERKENNLTKAERGGLDERDKVGFIKRQLVETRQITKHVAQ |
| | ILDARFNTEVNEKKQKIRTVKIITLKSNLVSNFRKEFRLYKVREINDYHHAHDAYLNAVVAKAILK |
| | KYPKLEPEFVYGDYQKYDLKRYISKSKNPKSIDKATQKYFFYSNLLNFFKEEVHYADGTIIKRENIE |
| | YSKDTGEIAWNKEKDFATVRKVLSCPQVNIVKKTEVQTGGFSKESILPKRNSDKLIARKKDWDPK |
| | KYGGFDSPTVAYSVLVVAKVEKGKSKKLKSVKELLGITIMERSSFEKNPIDFLEAKGYKEVRKDLI |
| | VKLPKYSLFELENGRKRMLASAGELQKGNELALPSKYVNFLYLASHYEKLKGSPEDNEQKQLFVE |
| | QHKHYLDEIIEQISEFSKRVILADANLDKVLSAYNKHRDKPIREQAENIIHLFTLTNLGAPAAFKYFD |
| | TTIDRKRYTSTKEVLDATFIHQSITGLYETRIDLSQLGGD |
| 31 | MTKPYSIGLDIGTNSVGWAVITDNYKVPSKKMKVLGNTSKKYVKKNLLGALLFESGITAEGRRLK |
| | RTARRRYTRRRNRILYLQEIFSTEMSKVDESFFQRLDDSFLVPDDKRDSKYPIFGNLVEEKTYHDEF |
| | PTIYHLRKYLADSTKKADLRLVYLALAHMIKYRGHFLIEGDFNSRNNDIKKNFQDFLDSYNAIFESD |
| | LSLENSKQLEEIVKDKISKSAKRERVLKLFPDEKSTGLFSEFLKLIVGNQADFKKHFDLEEKAPLQFS |
| | KDTYDEDLENLLGQIGDDFADLFLVAKKLYDAILLSGILTVTDPSTKAPLSASMIERYENHQKDLA |
| | ALKQFIQNNLQEKYDEVFSDQSKDGYAGYINGKTTQEAFYKYIKNLLSKLKGADYFLEKIEREDFL |
| | RKQRTFDNGSIPHQIHLQEMNAILRRQGEHYPFLKENREKIEKILTFRIPYYVGPLARGNRDFAWLT |
| | RNSDQAIRPWNFEEVVDQASSAEDFINKMTNYDLYLPEEKVLPKHSLLYETFAVYNELTKVKFIAE |
| | GLRDYQFLDSGQKKQIVTQLFKEKRKVTEKDIIQFLHNVDGYDGIELKGIEKQFNASLSTYHDILKII |
| | KDKEFMDDPKNGEILENIIHTLTIFEDREMIKQRLAQYDSIFDEKVIKALARRHYTGWGKLSAKLIN |
| | GIRDKQTNKTILDYLIDDDKINRNFMQLINDDGLSFKDIIQKAQVVGKTDDVKQVVQELPGSPAIKK |
| | GILQSIKIVDELVKVMGHAPESIVIEMARENQTTNQGKRNSQQRYKRIEEAIKNLDSNLNSKILKEYP |
| | TDNQALQNDRLFLYYLQNGKDMYTGEELDINQLSNYDIDHIIPQAFIKDDSLDNRVLTSSKDNRGK |
| | SDNVPSLEVVQKRKGFWQQLLNSKLISERKENNLTKAERGGLDERDKVGFIKRQLVETRQITKHVA |
| | QILDARFNTEVDENGKRIRKVKIITLKSNLVSNFRKEFGLYKVREINDYHHAHDAYLNAVVAKAIL |
| | KKYPKLEPEFVYGDYQKYDIKRYISKSRNPRSVDKATQKYFFYSNLLNFFKEEVHYADGTIIKRENI |
| | EYSKDTGEIAWNKEKDFATVRKVLSCPQVNIVKKTEVQTGGFSKESILPKRNSDKLIARKKDWDPK |
| | KYGGFDSPTVAYSVLVVAKVEKGKSKKLKSVKELLGITIMERSSFEKNPIDFLEAKGYKEVRKDLI |
| | VKLPKYSLFELENGRKRMLASAGELQKGNELALPSKYVNFLYLASHYEKLKGSPEDNEQKQLFVE |
| | QHKHYLDEIIEQISEFSKRVILADANLDKVLSAYNKHRDKPIREQAENIIHLFTLTNLGAPAAFKYFD |
| | TTIDRKRYTSTKEVLDATFIHQSITGLYETRIDLSQLGGD |
| 32 | MTKPYSIGLDIGTNSVGWAVITDSYKVPSKKMKVLGNTSKKYVKKNLLGALLFESGITAEGRRLK |
| | RTARRRYTRRRNRILYLQEIFSTEMAKIDESFFQRLDDSFLVPDDKRDSKYPIFGNLVEEKTYHDEFP |
| | TIYHLRKYLADSSKKADLRLVYLALAHMIKYRGHFLIEGDFNSRNNDIKKNFQNFLDSYNAIFESDL |
| | SLENSEQLEEIVKDKISKSAKRERVLKLFPDEKSTGLFSEFLKLIVGNQADFKKHFDLEEKAPLQFSK |
| | DTYDEDLENLLGQIGDDFADLFLVAKKLYDAILLSGILTVTDPSTKAPLSASMIERYENHQKDLAAL |
| | KQFIQNNLQEKYDEVFSDQSKDGYAGYIDGKTTQEAFYKYIKNLLSKFEGADYFLEKIEREDFLRK |
| | QRTFDNGSIPHQVHLQEMNAILHRQGEYYPFLQENKEKIQQILTFRIPYYVGPLARGNRDFAWLTR |
| | NSDQAIRPWNFEEVVDQASSAEDFINKMTNYDLYLPEEKVLPKHSLLYETFAVYNELTKVKFIAEG |
| | LRDYQFLDSGQKKQIVTQLFKEKRKVTEKDIIQFLHNVDGYDGIELKGIEKQFNASLSTYHDLLKII |
| | KDKEFMDDPKNEEILENIVHTLTIFEDREMIKQRLAQYDSLFDEKVIKALTRRHYTGWGKLSAKLIN |
| | GIYDKQSKKTILDYLIDDDKINRNFMQLINDDGLSFKEIIQKAQVVGKTDDVKQVVQELPGSPAIKK |
| | GILQSIKIVDELVKVMGHAPESIVIEMARENQTTNQGKRNSQQRYKRIEDAVKNLASELDGNILKEY |
| | PTDNQALQNDRLFLYYLQNGKDMYTGEPLDINQLSSYDIDHIVPQAFIKDDSLDNRVLTSSKDNRG |
| | KSDNVPSLEVVQKRKAFWQQLLDSKLISERKENNLTKAERGGLDERDKVGFIKRQLVETRQITKHV |
| | AQILDARFNTEVNEKGQKIRKVKIITLKSNLVSNFRKEFGLYKVREINDYHHAHDAYLNAVVAKAI |
| | LKKYPKLEPEFVYGDYQKYDIKRYISRSKDPKDVEKATQKYFFYSNLLNFFKEEVHYADGTIVKRE |
| | NIEYSKDTGEIAWNKEKDFATVRKVLSCPQVNIVKKTEVQTGGFSKESILPKRNSDKLIARKKDWD |
| | PKKYGGFDSPTVAYSVLVVAKVEKGKSKKLKSVKELLGITIMERSSFEKNPIDFLEAKGYKEVRKD |
| | LIVKLPKYSLFELENGRKRMLASAGELQKGNELALPSKYVNFLYLASHYEKLKGSPEDNEQKQLFV |
| | EQHKHYLDEIIEQISEFSKRVILADANLDKVLSAYNKHRDKPIREQAENIIHLFTLTNLGAPAAFKYF |
| | DTTIDRKRYTSTKEVLDATFIHQSITGLYETRIDLSQLGGD |
| 33 | MTKPYSIGLDIGTNSVGWAVITDSYKVPSKKMKVLGNTSKKYVKKNLLGALLFESGITAEGRRLK |
| | RTARRRYTRRRNRILYLQEIFSTEMAKIDESFFQRLDDSFLVPDDKRDSKYPIFGNLVEEKTYHDEFP |
| | TIYHLRKYLADSSKKADLRLVYLALAHMIKYRGHFLIEGDFNSRNNDIKKNFQDFLDSYNAIFESDL |
| | SLENSKQLEEIVKDKISKSAKRERVLKLFPDEKSTGLFSEFLKLIVGNQADFKKHFDLEEKAPLQFSK |
| | DTYDEDLENLLGQIGDDFADLFLVAKKLYDAILLSGILTVTDPSTKAPLSASMIERYENHQKDLAAL |
| | KQFIKSNLSEKYDEVFSDESKNGYAGYIENGVKQDEFYKYLKNTLSKIEGSDYFLDKIEREDFLRKQ |
| | RTFDNGSIPHQIHLQEMNAIIRRQGEHYPFLKENKEKIEKILTFRIPYYVGPLARGNRDFAWLTRNSD |
| | QAIRPWNFEEVVDKASSAEEFINKMTNYDLYLPEEKVLPKHSLLYETFAVYNELTKVKFIAEGLRD |
| | YQFLDSGQKKQIVNQLFKEKRKVTEKDIIHFLHNVDGYDGIELKGIEKQFNASLSTYHDLLKIIKDK |
| | EFMDDPKNEEILENIVHTLTIFEDREMIKQRLAQYDSLFDEKVIKALTRRHYTGWGKLSAKLINGIC |
| | DKKTGKTILDYLIDDGEINRNFMQLIHDDGLSFKEIIQNAQTFGKTDDVKQVVQELPGSPAIKKGIL |
| | QSIKIVDELVKVMGHAPESIVIEMARENQTTARGKKNSQQRYKRIEDSLKNLASGLDSNILKENPTD |
| | NIQLQNDRLFLYYLQNGKDMYTGEALDINQLSSYDIDHIIPQAFIKDDSLDNRVLTSSKDNRGKSDN |
| | VPSEEVVEKMKAFWQQLLDSKLISERKYNNLTKAERERDGLNELDKVGFIKRQLVETRQITKHVA |
| | QILDARFNTEVNEKKQKIRSVKIITLKSNLVSNFRKEFGLYKVREINDYHHAHDAYLNAVLAKAILK |
| | KYPKLEPEFVYGDYQKYDLKRYISKSKEPKDIEKATEKYFFYSNLLNFFKEEVHYADGTIVKRENIE |
| | YSKDTGEIAWNKEKDFATVRKVLSCPQVNIVKKTEVQTGGFSKESILPKRNSDKLIARKKDWDPK |
| | KYGGFDSPTVAYSVLVVAKVEKGKSKKLKSVKELLGITIMERSSFEKNPIDFLEAKGYKEVRKDLI |
| | VKLPKYSLFELENGRKRMLASAGELQKGNELALPSKYVNFLYLASHYEKLKGSPEDNEQKQLFVE |
| | QHKHYLDEIIEQISEFSKRVILADANLDKVLSAYNKHRDKPIREQAENIIHLFTLTNLGAPAAFKYFD |
| | TTIDRKRYTSTKEVLDATFIHQSITGLYETRIDLSQLGGD |
| 34 | MTKPYSIGLDIGTNSVGWAVITDSYKVPSKKMKVLGNTSKKYVKKNLLGALLFESGITAEGRRLK |
| | RTARRRYTRRRNRILYLQEIFSTEMNKIDESFFQRLDDSFLVPDDKRDSKYPIFGNLVEEKAYHDEF |
| | PTIYHLRKYLADSQKKADLRLVYLALAHMIKYRGHFLIEGDFNSRNNDIKKNFQDFLDSYNAIFES |
| | DLSLENSKQLEEIVKDKISKSAKKDRILKLFPGEKNSGIFSEFLKLIVGNQADFKKYFNLDEKAPLHF |
| | SKESYDEDLETLLGHIGDDYSDVFLKAKKLYDAILLSGILTVTDNETEAPLSSAMIMRYKEHEEDLA |
| | LLKAYIRKISLETYNEVFKDDTKNGYAGYIDGKTNQEDFYVYLKKLLTELKGADYFLDKINREDLL |
| | RKQRTFDNGSIPYQIHLQEMHAILRKQEKFYPFLAKNKERIEKILTFRIPYYVGPLARGNSDFAWSIR |
| | KRNEKITPWNFEDVIDKESSAEAFINRMTSFDLYLPEEKVLPKHSLLYETFTVYNELTKVRFIAESM |
| | RDYQFLDSKQKKDIVRLYFKGKRKVTDKDIIDYLHAIDGYDGIELKGIEKQFNSSLSTYHDLLNIIN |
| | DKEFLDDSSNEAIIEEIIHTLTIFEDREMIKQRLSKFENIFDKSVLKKLSRRHYTGWGKLSAKLINGIR |
| | DEKSGKTILDYLIDDGISNRNFMQLIHDDTLSFKKKIQKAQIIGDQDNIKQVVQSLPGSPAIKKGILQS |
| | IKIVDELVKVMGRNPESIVVEMARENQYTNQGKSNSQQRLKRLEESLKELGSKILKENVPVKLSKI |
| | DNNALQNERLYLYYLQNGKDMYTGQDLDIDRLSNYDIDHIIPQAFLKDNSIDNKVLVSSASNRGKS |
| | DDVPSLDVVKKRKGFWHQLLKSKLISQRKFDNLTKAERGGLTEADKAGFIQRQLVETRQITKHVA |
| | QILDERFNNEVTDKGKKIRKVKIITLKSNLVSQFRKEFGFYKVREINDYHHAHDAYLNAVVATALL |
| | KKYPQLAPEFVYGDYKQVKIDKNGKRTKSQIETSSDKATAKYFFYSNLLNFFKTKVRYADGTVLV |
| | RPVIEYSKDTGEIAWNKEKDFATVRKVLSCPQVNIVKKTEVQTGGFSKESILPKRNSDKLIARKKD |
| | WDPKKYGGFDSPTVAYSVLVVAKVEKGKSKKLKSVKELLGITIMERSSFEKNPIDFLEAKGYKEVR |
| | KDLIVKLPKYSLFELENGRKRMLASAGELQKGNELALPSKYVNFLYLASHYEKLKGSPEDNEQKQ |
| | LFVEQHKHYLDEIIEQISEFSKRVILADANLDKVLSAYNKHRDKPIREQAENIIHLFTLTNLGAPAAF |
| | KYFDTTIDRKRYTSTKEVLDATFIHQSITGLYETRIDLSQLGGD |
| 35 | MTKPYSIGLDIGTNSVGWAVITDNYKVPSKKMKVLGNTSKKYVKKNLLGALLFESGITAEGRRLK |
| | RTARRRYTRRRNRILYLQEIFATEMAKIDESFFQRLDDSFLVPDDKRDSKYPIFGNLVEEKTYHDEF |
| | PTIYHLRKYLADSSKKADLRLVYLALAHMIKYRGHFLIEGDFNSRNNDIKKNFQDFLDSYNAIFESD |
| | LSLENSKQLEEIVKDKISKSAKRERVLKLFPDEKSTGLFSEFLKLIVGNQADFKKHFDLEEKAPLQFS |
| | KDTYDEDLENLLGQIGDDFADLFLVAKKLYDAILLSGILTVTDPSTKAPLSASMIERYENHQKDLA |
| | ALKQFIQNNLQEKYDEVFSDQSKDGYAGYIDGKTTQEAFYKYIKNLLSKFEGADYFLDKIEREDFL |
| | RKQRTFDNGSIPHQIHLQEMNAILRRQGEHYPFLKENKEKIEKILTFRIPYYVGPLARGNRDFAWLT |
| | RNSDQAIRPWNFEEVVDKASSAEDFINKMTNYDLYLPEEKVLPKHSLLYETFAVYNELTKVKFIAE |
| | GLRDYQFLDSGQKKQIVNQLFKEKRKVTEKDIIHFLHNVDGYDGIELKGIEKQFNASLSTYHDLLKI |
| | IKDKEFMDDPKNEEILENIVHTLTIFEDREMIKQRLNQYDSLFDEKVIKALTRRHYTGWGKLSAKLI |
| | NGIRDKQTGKTILDYLIDDGYSNRNFMQLINDDGLSFKEIIQKAQVVGESDNLKQVVQNLPGSPAIK |
| | KGILQSIKIVDELVKVMGHDPEQIVIEMARENQTTNQGRRNSQQRYKKIENAIKNLDSNLNSKILKE |
| | YPTDNKALQNERLFLYYLQNGRDMYRDEELDINQLSNYDIDHIVPQAFIKDDSLDNRVLTSSKDNR |
| | GKSDNVPNIEIVEKMKGFWRQLLKSKLISQRKFDNLTKAERGGLTEHDKAGFIKRQLVETRQITKH |
| | VAQILDARFNTEVNEKNQKIRTVKIITLKSNLVSNFRKEFGLYKVREINDYHHAHDAYLNAVVAKA |
| | ILKKYPKLEPEFVYGDYQKYDIKRYISRSKDPKEIEKATEKYFFYSNLLNFFKEEVHYADGTIVKRE |
| | NVEYSNDTGEIAWNKEKDFATVRKVLSCPQVNIVKKTEVQTGGFSKESILPKRNSDKLIARKKDW |
| | DPKKYGGFDSPTVAYSVLVVAKVEKGKSKKLKSVKELLGITIMERSSFEKNPIDFLEAKGYKEVRK |
| | DLIVKLPKYSLFELENGRKRMLASAGELQKGNELALPSKYVNFLYLASHYEKLKGSPEDNEQKQLF |
| | VEQHKHYLDEIIEQISEFSKRVILADANLDKVLSAYNKHRDKPIREQAENIIHLFTLTNLGAPAAFKY |
| | FDTTIDRKRYTSTKEVLDATFIHQSITGLYETRIDLSQLGGD |
| 36 | MKKPYSIGLDIGTNSVGWAVVTDDYKVPAKKMKVLGNTDKSHIKKNLLGALLFEGGETAEGRRL |
| | KRTARRRYTRRRNRILYLQEIFSTEMAKIDESFFQRLDDSFLVPDDKRDSKYPIFGNLVKEKTYHDE |
| | FPTIYHLRKYLADSSKKADLRLVYLALAHMIKYRGHFLIEGEFNSRNNDIKKNFQEFLDSYNAIFES |
| | DLSLSHSEQVEEIVKDKISKSAKRERVLKLFPDEKSTGLFSEFLKLIVGNQADFKKHFDLEEKAPLQF |
| | SKDTYDEDLENLLGQIGDDFADLFLVAKKLYDAILLSGILTVTDPSTKAPLSASMIERYENHQKDLA |
| | ALKQFIKNNLSEKYDEVFSDQSKDGYAGYIDGKTTQEAFYKYIKNLLSKLEGADYFLDKIEREDFL |
| | RKQRTFDNGSIPHQIHLQEMNAIIRRQGEHYPFLKENKEKIEKILTFRIPYYVGPLARGNRDFAWLTR |
| | NSDQAIRPWNFEEIVDQASSAEDFINKMTNYDLYLPEEKVLPKHSLLYETFAVYNELTKVKFIAEGL |
| | RDYQFLDSGQKKQIVTQLFKEKRKVTEKDIIQFLHNVDGYDGIELKGIEKQFNASLSTYHDLLKIIK |
| | DKEFMDDPKNEEILENIVHTLTIFEDREMIKQRLAQYDSLFDEKVIKALTRRHYTGWGKLSAKLING |
| | ICDKQTGDTILDFLIDDDKINRNFMQLINDDGLSFKEIIQKAQVVGKTDDLKQVVQELPGSPAIKKGI |
| | LQSIKIVDELVKVMGHDPESIVIEMARENQTTNQGRRNSQQRYKKIEEAIKNLDSNLNSKILKEYPT |
| | NNQALQNDRLFLYYLQNGKDMYTGEPLDINQLSNYDIDHIVPQAFIKDDSLDNRVLTSSKDNRGKS |
| | DNVPSLEVVQKRKAFWQQLLDSKLISERKENNLTKAERGGLDERDKVGFIKRQLVETRQITKHVA |
| | QILDARFNTEVNDKKQKIRNVKIITLKSNLVSNFRKEFGLYKVREINDYHHAHDAYLNAVVAKAIL |
| | KKYPKLEPEFVYGDYQKYDIKRYISRSKDPKDIEKATEKYFFYSNLLNFFKEEVHYADGTIIKRENIE |
| | YSKDTGEIAWNKEKDFATVRKVLSCPQVNIVKKTEVQTGGFSKESILPKRNSDKLIARKKDWDPK |
| | KYGGFDSPTVAYSVLVVAKVEKGKSKKLKSVKELLGITIMERSSFEKNPIDFLEAKGYKEVRKDLI |
| | VKLPKYSLFELENGRKRMLASAGELQKGNELALPSKYVNFLYLASHYEKLKGSPEDNEQKQLFVE |
| | QHKHYLDEIIEQISEFSKRVILADANLDKVLSAYNKHRDKPIREQAENIIHLFTLTNLGAPAAFKYFD |
| | TTIDRKRYTSTKEVLDATFIHQSITGLYETRIDLSQLGGD |
| 37 | MTKPYSIGLDIGTNSVGWAVITDSYKVPSKKMKVLGNTSKKYVKKNLLGALLFESGITAEGRRLK |
| | RTARRRYTRRRNRILYLQEIFSTEMAKIDESFFQRLDDSFLVPDDKRDSKYPIFGNLVEEKTYHDEFP |
| | TIYHLRKYLADSTKKADLRLVYLALAHMIKYRGHFLIEGDFNSRNNDIKKNFQDFLDSYNAIFESD |
| | LSLENSKQLEEIVKDKISKSAKKDRLLKLFPGEKNSGIFSEFLKLIVGNQADFRKYFNLDEKASLHES |
| | KESYDEDLETLLGHIGDDYSDVFLKAKKLYDAILLSGILTVTDNETEAPLSSAMIMRYKEHEEDLAL |
| | LKAYIRKISLETYNEVFKDDTKNGYAGYIDGKTNQEDFYVYLKKLLTGLEGADYFLEKINREDLLR |
| | KQRTFDNGSIPYQIHLQEMRAIIDKQAKFYPFLAKNKERIEKILTFRIPYYVGPLARGNSDFAWSIRK |
| | RNEKITPWNFEDVIDKESSAEAFINRMTSFDLYLPEEKVLPKHSLLYETFTVYNELTKVRFIAESMR |
| | DYQFLDSKQKKDIVRLYFKGKRKVTDKDIIEYLHAIDGYDGIELKGIEKQFNSSLSTYHDLLNIINDK |
| | EFLDDSSNEAIIEEIIHTLTIFEDREMIKQRLSKFENIFDKSVLKKLSRRHYTGWGKLSAKLINGIRDE |
| | KSGKTILDYLIDDGVSNRNFMQLIHDDTLSFKKKIQKAQIIGDQDNIKQVVQSLPGSPAIKKGILQSI |
| | KIVDELVKVMGRNPEHIVVEMARENQFTNQGRRNSQQRLKRLEESLKELGSKILKENVPAKLSKID |
| | NNALQNDRLYLYYLQNGKDMYTGEELDIDRLSDYDIDHIIPQAFLKDDSIDNRVLVSSASNRGKSD |
| | DVPSLEVVKRRKNFWRQLLKSKLISQRKFDNLTKAERGGLTEKDKAGFIHRQLVETRQITKHVAQL |
| | LDSRFNTKRDDAGKVIREVKIITLKSSLVSQFRKDFKLYKVREINDYHHAHDAYLNAVVATAILKK |
| | YPKLEPEFVYGNYRDYQKYLMIKSDKDYGKATAKMFFYSNLMNFFKKEVRLSDGTVIVRPVIEYS |
| | KDTGEIAWNKEKDFATVRKVLSCPQVNIVKKTEVQTGGFSKESILPKRNSDKLIARKKDWDPKKY |
| | GGFDSPTVAYSVLVVAKVEKGKSKKLKSVKELLGITIMERSSFEKNPIDFLEAKGYKEVRKDLIVKL |
| | PKYSLFELENGRKRMLASAGELQKGNELALPSKYVNFLYLASHYEKLKGSPEDNEQKQLFVEQHK |
| | HYLDEIIEQISEFSKRVILADANLDKVLSAYNKHRDKPIREQAENIIHLFTLTNLGAPAAFKYFDTTID |
| | RKRYTSTKEVLDATFIHQSITGLYETRIDLSQLGGD |
| 38 | MTKPYSIGLDIGTNSVGWAVITDSYKVPSKKMKVLGNTSKKYVKKNLLGALLFESGITAEGRRLK |
| | RTARRRYTRRRNRILYLQEIFSTEMAKIDESFFQRLDDSFLVPDDKRDSKYPIFGNLVEEKTYHDEFP |
| | TIYHLRKYLADSSKKADLRLVYLALAHMIKYRGHFLIEGDFNSRNNDIKKNFQEFLDSYNAIFESDL |
| | SLENSKQLEEIVKDKISKSAKRERVLKLFPDEKSTGLFSEFLKLIVGNQADFKKHFDLEEKAPLQFSK |
| | DTYDEDLENLLGQIGDDFADLFLVAKKLYDAILLSGILTVTDPSTKAPLSASMIERYENHQKDLAAL |
| | KQFIKNNLSEKYDEVFSDQSKDGYAGYIDGKTTQEAFYKYIKNLLTKFEGADYFLEKIKREDFLRK |
| | QRTFDNGSIPYQVHLQEMKAILSRQGDYYPFLKENKEKIEKILTFRIPYYVGPLARGNRDFAWLTRN |
| | SDQAIRPWNFEEVVDKASSAEDFINKMTNYDLYLPEEKVLPKHSLLYETFAVYNELTKVKFIAEGL |
| | RDYQFLDSGQKKQIVNQLFKEKRKVTEKDIIHYLHNVDGYDGIELKGIEKQFNASLSTYHDLLKIIK |
| | DKEFMDDAKNEEILENIIHTLTIFEDREMIKQRLAQYDSLFDEKVIKALTRRHYTGWGKLSAKLING |
| | IYDKQSKKTILDYLIDDDKINRNFMQLINDDGLSFKEIIQKAQVVGKTDDLKQVVQNLPGSPAIKKG |
| | ILQSIKIVDELVKVMGHAPESIVIEMARENQTTNQGKRNSQQRYKRIEDAVKNLASELDGNILKEYP |
| | TNNQALQNDRLFLYYLQNGKDMYTGEPLDINQLSSYDIDHIIPQAFIKDDSLDNRVLTSSKDNRGK |
| | SDNVPSLEVVQKRKAFWQQLLDSKLISERKFNNLTKAERGGLDERDKVGFIKRQLVETRQITKHVA |
| | QILDARFNTEVNEKNQKIRTVKIITLKSNLVSNFRKEFGLYKVREINDYHHAHDAYLNAVVAKAIL |
| | KKYPKLEPEFVYGDYQKYDLKRYISKSRNPKAIDKATQKYFFYSNLLNFFKEEVHYADGTIIKRENI |
| | EYSKDTGEIAWNKEKDFATVRKVLSCPQVNIVKKTEVQTGGFSKESILPKRNSDKLIARKKDWDPK |
| | KYGGFDSPTVAYSVLVVAKVEKGKSKKLKSVKELLGITIMERSSFEKNPIDFLEAKGYKEVRKDLI |
| | VKLPKYSLFELENGRKRMLASAGELQKGNELALPSKYVNFLYLASHYEKLKGSPEDNEQKQLFVE |
| | QHKHYLDEIIEQISEFSKRVILADANLDKVLSAYNKHRDKPIREQAENIIHLFTLTNLGAPAAFKYFD |
| | TTIDRKRYTSTKEVLDATFIHQSITGLYETRIDLSQLGGD |
| 39 | MTKPYSIGLDIGTNSVGWAVITDSYKVPSKKMKVLGNTSKKYVKKNLLGALLFESGITAEGRRLK |
| | RTARRRYTRRRNRILYLQEIFSTEMAKIDESFFQRLDDSFLVPDDKRDSKYPIFGNLVKEKTYHDEF |
| | PTIYHLRKYLADSSKKADLRLVYLALAHMIKYRGHFLIEGDFNSRNNDIKKNFQDFLDSYNAIFESD |
| | LSLENSKQLEEIVKDKISKSAKKDRLLKLFPGEKNSGIFSEFLKLIVGNQADFRKYFNLDEKASLHES |
| | KESYDEDLETLLGHIGDDYSDVFLKAKKLYDAILLSGILTVTDNETEAPLSSAMIMRYKEHEEDLAL |
| | LKAYIRKISLETYNEVFKDDTKNGYAGYIDGKTNQEDFYVYLKKLLTGLEGADYFLEKINREDLLR |
| | KQRTFDNGSIPYQIHLQEMRAIIDKQAKFYPFLAKNKERIEKILTFRIPYYVGPLARGNSDFAWSIRK |
| | RNEKITPWNFEDVIDKESSAEAFINRMTSFDLYLPEEKVLPKHSLLYETFTVYNELTKVRFIAESMR |
| | DYQFLDSKQKKDIVRLYFKGKRKVTDKDIIDYLHAIDGYDGIELKGIEKQFNSSLSTYHDLLNIIND |
| | KEFLDDSSNEAIIEEIIHTLTIFEDREMIKQRLSKFENIFDKSVLKKLSRRHYTGWGKLSAKLINGIRD |
| | EKSGKTILDYLIDDGISNRNFMQLIHDDTLSFKKKIQKEQYIEDQDNIKQVVQSLPGSPAIKKGILQSI |
| | KIVDELVKVMGGRKPESIVVEMARENQYTNQGKSNSQQRLKRLEESLKELGSKILKENVPAKLSKI |
| | DNNALQNERLYLYYLQNGKDMYTGDDLDIDRLSNYDIDHIIPQAFLKDNSIDNKVLVSSASNRGKS |
| | DDVPSLDVVKKRKTFWHQLLKSKLISQRKFDNLTKAERGGLTEDDKAGFIRRQLVETRQITKHVA |
| | QLLDSRFNTKRDDNDKVIRDVKIITLKSSLVSQFRKDFKLYKVREINDYHHAHDAYLNAVVATAIL |
| | KKYPKLEPEFVYGEYKKYDVRKFIAKSDKDLGKATAKLFFYSNLMNFFKKEVRLADGTVVVRPVI |
| | EYSKDTGEIAWNKEKDFATVRKVLSCPQVNIVKKTEVQTGGFSKESILPKRNSDKLIARKKDWDPK |
| | KYGGFDSPTVAYSVLVVAKVEKGKSKKLKSVKELLGITIMERSSFEKNPIDFLEAKGYKEVRKDLI |
| | VKLPKYSLFELENGRKRMLASAGELQKGNELALPSKYVNFLYLASHYEKLKGSPEDNEQKQLFVE |
| | QHKHYLDEIIEQISEFSKRVILADANLDKVLSAYNKHRDKPIREQAENIIHLFTLTNLGAPAAFKYFD |
| | TTIDRKRYTSTKEVLDATFIHQSITGLYETRIDLSQLGGD |
| 40 | MNKPYSIGLDIGTNSVGWAVITDDYKVPSKKMKVLGNTSKKYVKKNLLGALLFESGITAEGRRLK |
| | RTARRRYTRRRNRILYLQEIFSTEMNKIDEGFFQRLDDSFLVPDDKRDSKYPIFGNLVEEKAYHDEF |
| | PTIYHLRKYLADSSKKADLRLVYLALAHMIKYRGHFLIEGDFNSRNNDIKKNFQEFLDSYNAIFESD |
| | LSLENSKQLEEIVKDKISKSAKRERVLRLFPDEKSTGLFSEFLKLIVGNQADFKKHFDLEEKAPLQFS |
| | KDTYDEDLENLLGQIGDDFADLFLVAKKLYDAILLSGILTVTDPSTKAPLSASMIERYENHQKDLA |
| | ALKQFIQNNLQEKYDEVFSDQSKDGYAGYIDGKTTQEAFYKYIKNLLAKFEGADYFLEKIKREDFL |
| | RKQRTFDNGTIPYQVHLAEMRAILHRQGDYYPFLKENKEKIEKILTFRIPYYVGPLARGNRDFAWL |
| | TRNSDQAIRPWNFEEIVDQASSAEEFINKMTNYDLYLPEEKVLPKHSLLYETFAVYNELTKVKFIAE |
| | GLRDYQFLDSGQKKQIVNQLFKEKRKVTEKDIIQFLHNVDGYDGIELKGIEKQFNASLSTYHDLLKI |
| | IKDKDFMDDPKNEEILENIVHTLTIFEDREMIKQRLAQYDSLFDEKVIKALTRRHYTGWGKLSAKLI |
| | NGIYDKQSKKTILDYLIDDGKINRNFMQLINDDGLSFKDIIQKAQVVGKTDDVKQVVQELPGSPAIK |
| | KGILQSIKIVDELVKVMGHAPESIVIEMARENQTTARGKKNSQQRYKRIEDALKNLAPGLDSNILKE |
| | HPTDNIQLQNDRLFLYYLQNGKDMYTGEPLDINQLSSYDIDHIVPQAFIKDDSLDNRVLTSSKDNR |
| | GKSDNVPSLEVVQKRKAFWQQLLDSKLISERKENNLTKAERGGLDERDKVGFIKRQLVETRQITKH |
| | VAQILDARFNTEVNEENQRIRKVKIITLKSNLVSNFRKEFGLYKVREINDYHHAHDAYLNAVVAKA |
| | ILKKYPKLEPEFVYGDYQKYDIKRYISRSKDPKDIEKATEKYFFYSNLLNFFKEEVHYADGTIIKREN |
| | IEYSKDTGEIAWNKEKDFATVRKVLSCPQVNIVKKTEVQTGGFSKESILPKRNSDKLIARKKDWDP |
| | KKYGGFDSPTVAYSVLVVAKVEKGKSKKLKSVKELLGITIMERSSFEKNPIDFLEAKGYKEVRKDL |
| | IVKLPKYSLFELENGRKRMLASAGELQKGNELALPSKYVNFLYLASHYEKLKGSPEDNEQKQLFVE |
| | QHKHYLDEIIEQISEFSKRVILADANLDKVLSAYNKHRDKPIREQAENIIHLFTLTNLGAPAAFKYFD |
| | TTIDRKRYTSTKEVLDATFIHQSITGLYETRIDLSQLGGD |
| 41 | MTKPYSIGLDIGTNSVGWAVITDSYKVPSKKMKVLGNTSKKYVKKNLLGALLFESGITAEGRRLK |
| | RTARRRYTRRRNRILYLQEIFSTEMAKIDESFFQRLDDSFLVPDDKRDSKYPIFGNLVEEKTYHDEFP |
| | TIYHLRKYLADSQKKADLRLVYLALAHMIKYRGHFLIEGDFNSRNNDIKKNFQDFLDSYNAIFESD |
| | LSLENSKQLEEIVKDKISKSAKRERVLKLFPDEKSTGLFSEFLKLIVGNQAEFKKHFDLEEKAPLQFS |
| | KDTYDEDLENLLGQIGDDFADLFLVAKKLYDAILLSGILTVTDPSTKAPLSASMIERYENHQKDLA |
| | ALKQFIKSNLSEKYDEVFSDQSKDGYAGYIDGKTTQEAFYKYIKNLLSKFEGADYFLDKIEREDFLR |
| | KQRTFDNGSIPHQIHLQEMNAILRRQGEHYPFLKENKEKIEKILTFRIPYYVGPLARGNRDFAWLTR |
| | NSDQAIRPWNFEEVVDQASSAEEFINKMTNYDLYLPEEKVLPKHSLLYETFAVYNELTKVKFIAEG |
| | LRDYQFLDSGQKKQIVNQLFKEKRKVTEKDIIQFLHNVDGYDGIELKGIEKQFNASLSTYHDLLKII |
| | KDKEFMDDPKNEEILENIVHTLTIFEDREMIKQRLAQYDSLFDEKVIKALTRRHYTGWGKLSAKLIN |
| | GICDKQTNKTILDYLIDDDKINRNFMQLIHDDGLSFKDIIQKAQVVGKTDDVKQVVQELPGSPAIKK |
| | GILQSIKIVDELVKVMGHAPESIVIEMARENQTTNQGRRNSQQRYKKIEEAIKNLDSNLNSKILKEYP |
| | TDNQALQNDRLFLYYLQNGKDMYTDEELDINQLSNYDIDHIVPQAFIKDDSLDNRVLTSSKDNRG |
| | KSDNVPSLEVVQKRKGFWQQLLDSKLISEHKFNNLTKAERGGLDERDKVGFIKRQLVETRQITKH |
| | VAQILDARFNTEVDENNQKIRTVKIITLKSNLVSNFRKEFGLYKVREINDYHHAHDAYLNAVVAKA |
| | ILKKYPKLEPEFVYGDYQKYDLKRYISKSKDPKEIEKATEKYFFYSNLLNFFKEEVHYADGTIIKRE |
| | NIEYSKDTGEIAWNKEKDFATVRKVLSCPQVNIVKKTEVQTGGFSKESILPKRNSDKLIARKKDWD |
| | PKKYGGFDSPTVAYSVLVVAKVEKGKSKKLKSVKELLGITIMERSSFEKNPIDFLEAKGYKEVRKD |
| | LIVKLPKYSLFELENGRKRMLASAGELQKGNELALPSKYVNFLYLASHYEKLKGSPEDNEQKQLFV |
| | EQHKHYLDEIIEQISEFSKRVILADANLDKVLSAYNKHRDKPIREQAENIIHLFTLTNLGAPAAFKYF |
| | DTTIDRKRYTSTKEVLDATFIHQSITGLYETRIDLSQLGGD |
| 42 | MTKPYSIGLDIGTNSVGWAVITDSYKVPSKKMKVLGNTSKKYVKKNLLGALLFESGITAEGRRLK |
| | RTARRRYTRRRNRILYLQEIFSTEMAKIDESFFQRLDDSFLVPDDKRDSKYPIFGNLVKEKTYHDEF |
| | PTIYHLRKYLADSSKKADLRLVYLALAHMIKYRGHFLIEGDFNSRNNDIKKNFQDFLDSYNAIFESD |
| | LSLENSKQLEEIVKDKISKSAKRERVLKLFPDEKSTGLFSEFLKLIVGNQADFKKHFDLEEKAPLQFS |
| | KDTYDEDLENLLGQIGDDFADLFLVAKKLYDAILLSGILTVTDPSTKAPLSASMIERYENHQKDLA |
| | QLKQFVKTNLPEKYDEVFSDQSKDGYAGYIDGKTTQEAFYVYLKKLLTGLEGADYFLEKIKREDF |
| | LRKQRTFDNGSIPYQVHLQEMQAILHRQGEYYPFLKENKEKIQQILTFRIPYYVGPLARGNRDFAW |
| | LTRNSDQAIRPWNFEEIVDKASSAEEFINKMTNYDLYLPEEKVLPKHSLLYETFAVYNELTKVKFIA |
| | EGLRDYQFLDSGQKKQIVNQLFKDKRKVTEKDIIHYLHNVDGYDGIELKGIEKQFNASLSTYHDLL |
| | KIIKDKEFMDDPKNEEILENIVHTLTIFEDREMIKQRLAQYDSLFDEKVIKALTRRHYTGWGKLSAK |
| | LINGICDKQTNKTILDYLIDDDKINRNFMQLINDDGLSFKDIIQKAQVVGKTDDVKQVVQELPGSPA |
| | IKKGILQSIKIVDELVKVMGHAPESIVIEMARENQTTARGKKNSQQRYKRIEDALKNLAPGLDSNIL |
| | KENPTDNIQLQNDRLFLYYLQNGKDMYTGKELDINQLSNYDIDHIIPQAFIKDDSLDNRVLTSSKDN |
| | RGKSDNVPSIEVVQKRKAFWQQLLDSKLISERKENNLTKAERERGGLNELDKVGFIKRQLVETRQI |
| | TKHVAQILDAKFNTEKDENGKRIRKVKIITLKSNLVSNFRKEFGLYKVREINDYHHAHDAYLNAVV |
| | AKAILKKYPKLEPEFVYGDYQKYDIKRYISRSKDPKDIEKATEKYFFYSNLLNFFKEEVHYADGTII |
| | KRENIEYSKDTGEIAWNKEKDFATVRKVLSCPQVNIVKKTEVQTGGFSKESILPKRNSDKLIARKK |
| | DWDPKKYGGFDSPTVAYSVLVVAKVEKGKSKKLKSVKELLGITIMERSSFEKNPIDFLEAKGYKEV |
| | RKDLIVKLPKYSLFELENGRKRMLASAGELQKGNELALPSKYVNFLYLASHYEKLKGSPEDNEQK |
| | QLFVEQHKHYLDEIIEQISEFSKRVILADANLDKVLSAYNKHRDKPIREQAENIIHLFTLTNLGAPAA |
| | FKYFDTTIDRKRYTSTKEVLDATFIHQSITGLYETRIDLSQLGGD |
| 43 | MNKPYSIGLDIGTNSVGWAVVDDDYKVPAKKMRVFGDTDRSHIKKNLLGTLLFDSGETAEGRRL |
| | KRTARRRYTRRRNRILYLQEIFAEEMSKVDDSFFHRLDESFLTDDDKNFDSHPIFGNKAEEDAYHQ |
| | KFPTIYHLRKHLADSTEKADLRLVYLALAHMIKFRGHFLIEGQLNAENTDVQKLFADFVGVYDRTF |
| | DDSHLSEITVDASSILTEKISKSRRLENLITHYPKEKKNTLFGNLIALALGLQPNFKTNFKLSEDAKL |
| | QFSKDTYEEDLEELLGKIGDDYADLFTSAKNLYDAILLSGILTVDDESTKAPLSASMVKRYAEHHE |
| | DLEKLKEFIKANKPELYHDIFKDETKNGYAGYIENGVKQDEFYKYLKNTLSKIAGSDYFLDKIERE |
| | DFLRKQRTFDNGSIPHQIHLQEMHAILRRQGDYYPFLKENQDRIEKILTFRIPYYVGPLARKDSRFA |
| | WANYHSEEKITPWNFDKVIDKEKSAEKFITRMTSNDLYLPEEKVLPKHSLLYETYTVYNELTKVKY |
| | VNEQGNATFFDSNIKQEIFEQLFKTKRKVTKKDIINYLENAYCIDDVEIMGVEDRFNASLGTYHDLL |
| | KIIQDKSFLDDKKNEEIIEDVVLTLTLFEDRDMIRQRLQKYSDIFTKQQLKKLERRHYTGWGRLSYK |
| | LINGIRNKENNKTILDFLIDDGRANRNFMQLINDDSLSFKTIIQEAQVVGDVDDIETVVHDLPGSPAI |
| | KKGILQSIKIVDELVKVMGHNPDNIVIEMARENQTTNRGRSQSQQRLKKLQESLKELGSSILNKEKP |
| | SYIEDKVENSHLQNDKLFLYYIQNGKDMYTGEELDIDHLSDYDIDHIIPQAFIKDDSIDNRVLTSSAK |
| | NRGKSDDVPSLDIVRDRKAEWIRLYKSKLISKRKFDNLTKAERGGLTENDKAGFIKRQLVETRQIT |
| | KHVAQILDARFNTERDENDKVIRDVKVITLKSNLVSQFRKEFGFYKVREINDYHHAHDAYLNAVV |
| | GTALLKKYPKLAPEFVYGEYQKYDVRKFISTKDNKDIGKATAKYFFYSNLLNFFKEEVRYANGTII |
| | VRPVIEYSKDTGEIAWNKEKDFATVRKVLSCPQVNIVKKTEVQTGGFSKESILPKRNSDKLIARKKD |
| | WDPKKYGGFDSPTVAYSVLVVAKVEKGKSKKLKSVKELLGITIMERSSFEKNPIDFLEAKGYKEVR |
| | KDLIVKLPKYSLFELENGRKRMLASAGELQKGNELALPSKYVNFLYLASHYEKLKGSPEDNEQKQ |
| | LFVEQHKHYLDEIIEQISEFSKRVILADANLDKVLSAYNKHRDKPIREQAENIIHLFTLTNLGAPAAF |
| | KYFDTTIDRKRYTSTKEVLDATFIHQSITGLYETRIDLSQLGGD |
| 44 | MTKPYSIGLDIGTNSVGWAVITDNYKVPSKKMKVLGNTSKKYVKKNLLGALLFESGITAEGRRLK |
| | RTARRRYTRRRNRILYLQEIFSTEMNKIDEGFFQRLDDSFLVPDDKRDSKYPIFGNLVEEKTYHDEF |
| | PTIYHLRKYLADSSKKADLRLVYLALAHMIKYRGHFLIEGDFNSRNNDIKKNFQDFLDSYNAIFESD |
| | LSLENSKQLEEIVKDKISKSAKRERVLKLFPDEKSTGLFSEFLKLIVGNQADFKKHFDLEEKAPLQFS |
| | KDTYDEDLENLLGQIGDDFADLFLVAKKLYDAILLSGILTVTDPSTKAPLSASMIERYENHQKDLA |
| | ALKQFIQNNLQEKYDEVFSDQSKDGYAGYINGKTTQEAFYKYIKNLLSKFEGADYFLDKIEREDFL |
| | RKQRTFDNGSIPHQIHLQEMNAIIRRQGEHYPFLKENKEKIEKILTFRIPYYVGPLARGNRDFAWLTR |
| | NSDQAIRPWNFEEIVDKASSAEDFINKMTNYDLYLPEEKVLPKHSLLYETFAVYNELTKVKFIAEGL |
| | RDYQFLDSGQKKQIVNQLFKEKRKVTEKDIIHYLHNVDGYDGIELKGIEKQFNASLSTYHDLLKIIK |
| | DKEFMDDPKNEEILENIVHTLTIFEDREMIKQRLAQYDSLFDEKVIKALTRRHYTGWGKLSAKLING |
| | IYDKQSKKTILDYLIDDDKINRNFMQLINDDGLSFKEIIQKAQVVGKTNDVKQVVQELPGSPAIKKG |
| | ILQSIKIVDELVKVMGGDPEHIVVEMARENQFTNQGRRNSQQRYKKIEDAIKNLDSNLNSKILKEYP |
| | TDNQALQNDRLFLYYLQNGKDMYTGEELDINQLSNYDIDHIVPQAFIKDDSLDNRVLTSSKDNRG |
| | KSDNVPSLEVVQKRKAFWQQLLDSKLISERKENNLTKAERGGLDERDKVGFIRRQLVETRQITKHV |
| | AQILDARFNTEVNEKGKKIRKVKIITLKSNLVSNFRKEFGLYKVREINDYHHAHDAYLNAVVAKAI |
| | LKKYPKLEPEFVYGDYQKYDIRDFIKAKRKTTDDKATAKYFFYSNLLNFFKKEVRYADGTVIVRPI |
| | VEYSKDTGEIAWNKEKDFATVRKVLSCPQVNIVKKTEVQTGGFSKESILPKRNSDKLIARKKDWDP |
| | KKYGGFDSPTVAYSVLVVAKVEKGKSKKLKSVKELLGITIMERSSFEKNPIDFLEAKGYKEVRKDL |
| | IVKLPKYSLFELENGRKRMLASAGELQKGNELALPSKYVNFLYLASHYEKLKGSPEDNEQKQLFVE |
| | QHKHYLDEIIEQISEFSKRVILADANLDKVLSAYNKHRDKPIREQAENIIHLFTLTNLGAPAAFKYFD |
| | TTIDRKRYTSTKEVLDATFIHQSITGLYETRIDLSQLGGD |
| 45 | MTKPYSIGLDIGTNSVGWAVITDNYKVPSKKMKVLGNTSKKYVKKNLLGALLFESGITAEGRRLK |
| | RTARRRYTRRRNRILYLQEIFSTEMSKVDENFFQRLDDSFLVPDDKRDSKYPIFGNLVEEKTYHDEF |
| | PTIYHLRKYLADSSKKADLRLVYLALAHMIKYRGHFLIEGDFNSRNNDIKKNFQQFLDSYNAIFESD |
| | LSLENSAQVEEIVKDKISKSAKRERVLKLFPDEKSTGLFSEFLKLIVGNQADFKKHFNLEEKAPLHES |
| | KDTYDEDLENLLGQIGDDFADLFLVAKKLYDAILLSGILTVTDPSTKAPLSASMIERYENHQKDLAT |
| | LKQFIKNNLSEKYDEVFSDQSKDGYAGYIDGKTTQEAFYKYIKNLLSKFEGADYFLDKIERENFLR |
| | KQRTFDNGSIPHQIHLQEMHAILHRQGEHYPFLKENKEKIEKILTFRIPYYVGPLARGNRDFAWLTR |
| | NSDQAIRPWNFEEVVDKASSAEDFINKMTNYDLYLPEEKVLPKHSLLYETFAVYNELTKVKFIAEG |
| | LRDYQFLDSGQKKQIVNQLFKEKRKVTEKDIIQFLHNVDGYDGIELKGIEKQFNASLSTYHDLLKII |
| | KDKEFMDDPKNEEILENIIHTLTIFEDREMIKQRLAQYDSLFDEKVIKALTRRHYTGWGKLSAKLIN |
| | GIRDKKTGKTILDYLIDDGKINRNFMQLIHDDGLSFKEIIQKAQVVGDTDDLKQVVQELPGSPAIKK |
| | GILQSIKIVDELVKVMGHNPENIVVEMARENQFTNQGRRNSQQRYKKIKDAIKNLDSNLNSKILKE |
| | YPTNNQALQNDRLFLYYLQNGKDMYTGEALDINQLSNYDIDHIIPQAFIKDDSLDNRVLTSSKDNR |
| | GKSDNVPSIEVVEKMKAFWQQLLDSKLISERKENNLTKAERERGGLNELDKVGFIKRQLVETRQIT |
| | KHVAQILDARFNTEVNEKKQKIRSVKIITLKSNLVSNFRKEFGLYKVREINDYHHAHDAYLNAVVA |
| | KAILKKYPKLEPEFVYGDYQKYDVRKFISTKDNDEIGKATAKYFFYSNLLNFFKTKVRYADGTVIV |
| | RPVIEYSKDTGEIAWNKEKDFATVRKVLSCPQVNIVKKTEVQTGGFSKESILPKRNSDKLIARKKD |
| | WDPKKYGGFDSPTVAYSVLVVAKVEKGKSKKLKSVKELLGITIMERSSFEKNPIDFLEAKGYKEVR |
| | KDLIVKLPKYSLFELENGRKRMLASAGELQKGNELALPSKYVNFLYLASHYEKLKGSPEDNEQKQ |
| | LFVEQHKHYLDEIIEQISEFSKRVILADANLDKVLSAYNKHRDKPIREQAENIIHLFTLTNLGAPAAF |
| | KYFDTTIDRKRYTSTKEVLDATFIHQSITGLYETRIDLSQLGGD |
| 46 | MTKPYSIGLDIGTNSVGWAVITDSYKVPSKKMKVLGNTSKKYVKKNLLGALLFESGITAEGRRLK |
| | RTARRRYTRRRNRILYLQEIFSTEMAKIDESFFQRLDDSFLVPDDKRDSKYPIFGNLVEEKVYHDEF |
| | PTIYHLRKYLADSSKKADLRLVYLALAHMIKYRGHFLIEGDFNSRNNDIKKNFQDFLDSYNAIFESD |
| | LSLENSKQLEEIVKDKISKSAKRERVLKLFPDEKSTGLFSEFLKLIVGNQADFKKHFDLEEKAPLQFS |
| | KDTYDEDLENLLGQIGDDFADLFLVAKKLYDAILLSGILTVTDPSTKAPLSASMIERYENHQKDLA |
| | ALKQFIQNNLQEKYDEVFSDQSKDGYAGYIDGKTTQEAFYKYIKNLLSKFEGADYFLEKIEREDFL |
| | RKQRTFDNGSIPHQIHLQEMNAILRRQGEYYPFLKENKEKIQQILTFRIPYYVGPLARGNRDFAWLT |
| | RNSDQAIRPWNFEEVVDKASSAEDFINKMTNYDLYLPEEKVLPKHSLLYETFAVYNELTKVKFIAE |
| | GLRDYQFLDSGQKKQIVNQLFKEKRKVTEKDIIHYLHNVDGYDGIELKGIEKQFNASLSTYHDLLKI |
| | IKDKEFMDDPKNEEILENIVHTLTIFEDREMIKQRLAQYDSLFDEKVIKALTRRHYTGWGKLSAKLI |
| | NGIYDKQSKKTILDYLIDDDKINRNFMQLINDDGLSFKEIIQKAQVVGKTDDVKQVVQELPGSPAIK |
| | KGILQSIKIVDELVKVMGHAPESIVIEMARENQTTARGKKNSQQRYKRIEDALKNLAPGLDSNILKE |
| | NPTDNIQLQNDRLFLYYLQNGKDMYTGESLDINQLSNYDIDHIIPQAFIKDDSLDNRVLTSSKDNRG |
| | KSDNVPSIEIVEKMKGFWQQLLNSKLISERKFNNLTKAERGGLSELDKAGFIKRQLVETRQITKHVA |
| | QILDARYNEEVDNNQKIRKVKIITLKSNLVSNFRKEFGLYKVREINDYHHAHDAYLNAVVAKAILK |
| | KYPKLEPEFVYGDYQKYDIKRYISKSKDPKDIEKATQKYFFYSNLLNFFKEEVHYADGTIIKRENIE |
| | YSKDTGEIAWNKEKDFATVRKVLSCPQVNIVKKTEVQTGGFSKESILPKRNSDKLIARKKDWDPK |
| | KYGGFDSPTVAYSVLVVAKVEKGKSKKLKSVKELLGITIMERSSFEKNPIDFLEAKGYKEVRKDLI |
| | VKLPKYSLFELENGRKRMLASAGELQKGNELALPSKYVNFLYLASHYEKLKGSPEDNEQKQLFVE |
| | QHKHYLDEIIEQISEFSKRVILADANLDKVLSAYNKHRDKPIREQAENIIHLFTLTNLGAPAAFKYFD |
| | TTIDRKRYTSTKEVLDATFIHQSITGLYETRIDLSQLGGD |
| 47 | MTKPYSIGLDIGTNSVGWAVITDSYKVPSKKMKVLGNTSKKYVKKNLLGALLFESGITAEGRRLK |
| | RTARRRYTRRRNRILYLQEIFSTEMAKIDESFFQRLDDSFLVPDDKRDSKYPIFGNLVKEKTYHDEF |
| | PTIYHLRKYLADSSKKADLRLVYLALAHMIKYRGHFLIEGDFNSRNNDIKKNFQDFLDSYNAIFESD |
| | LSLENSKQLEEIVKDKISKSAKRERVLKLFPDEKSTGLFSEFLKLIVGNQADFKKHFDLEEKAPLQFS |
| | KDTYDEDLENLLGQIGDGFADLFLVAKKLYDAILLSGILTVTDPSTKAPLSASMIERYENHQKDLSA |
| | LKQFIKRNLSEKYDEVFSDQSKDGYAGYINGKTTQEAFYKYIKNLLSKFEGADYFLDKIEREDFLR |
| | KQRTFDNGSIPHQIHLQEMNAIIRRQGEHYPFLQENKEKIEKILTFRIPYYVGPLARGNRDFAWLTR |
| | NSDQAIRPWNFEEVVDKARSAEDFINKMTNYDLYLPEEKVLPKHSLLYETFAVYNELTKVKFIAEG |
| | LRDYQFLDSGQKKQIVNQLFKEKRKVTEKDIIQFLHNVDGYDGIELKGIEKQFNASLSTYHDLLKII |
| | KDKEFMDDPKNEEILENIIHTLTIFEDREMIKQRLAQYDSLFDEKVIKALTRRHYTGWGKLSAKLIN |
| | GIYDKQSKKTILDYLIDDGEINRNFMQLIHDDGLSFKEIIQKAQVVGETDDLRQVVQNLPGSPAIKK |
| | GILQSIKLVDELVKVMGHEPEQIVIEMARENQTTNQGKRNSQQRYKRIEDGVKNLASDLNGNILKE |
| | YPTDNQALQNDRLFLYYLQNGKDMYTGEALDINQLSNYDIDHIVPQAFIKDDSLDNRVLTSSKDN |
| | RGKSDNVPSIEVVQKRKAFWQQLLDSKLISERKFNNLTKAERGGLDERDKVGFIKRQLVETRQITK |
| | HVAQILDARFNTEVNEKGKRIRKVKIITLKSNLVSNFRKEFGLYKVREINDYHHAHDAYLNAVVAK |
| | AILKKYPKLEPEFVYGDYQKYDIKRYISRSKDPKDIEKATEKYFFYSNLLNFFKEEVHYADGTIIKRE |
| | NIEYSKDTGEIAWNKEKDFATVRKVLSCPQVNIVKKTEVQTGGFSKESILPKRNSDKLIARKKDWD |
| | PKKYGGFDSPTVAYSVLVVAKVEKGKSKKLKSVKELLGITIMERSSFEKNPIDFLEAKGYKEVRKD |
| | LIVKLPKYSLFELENGRKRMLASAGELQKGNELALPSKYVNFLYLASHYEKLKGSPEDNEQKQLFV |
| | EQHKHYLDEIIEQISEFSKRVILADANLDKVLSAYNKHRDKPIREQAENIIHLFTLTNLGAPAAFKYF |
| | DTTIDRKRYTSTKEVLDATFIHQSITGLYETRIDLSQLGGD |
| 48 | MKKPYSIGLDIGTNSVGWAVITDSYKVPSKKMKVLGNTSKKYVKKNLLGALLFESGITAEGRRLK |
| | RTARRRYTRRRNRILYLQEIFAEEMNKIDESFFHRLDDSFLVPEDKRGSKYPIFATLEEEKEYHKQFP |
| | TIYHLRKHLADSKEKTDLRLIYLALAHMIKYRGHFLYEESFDIKNNDIQKIFNEFISIYDNTFEGSSLS |
| | GQNAQVEAIFTDKISKSAKRERVLKLFPDEKSTGLFSEFLKLIVGNQADFKKHFDLEEKAPLQFSKD |
| | TYDEDLENLLGQIGDDFADLFLVAKKLYDAILLSGILTVTDPSTKAPLSASMIERYENHQKDLAALK |
| | QFIQNNLQEKYDEVFSDQSKDGYAGYINGKTTQEAFYKYIKNLLNKVEGADYFLDKIEREDFLRKQ |
| | RTFDNGSIPHQIHLQEMHAILQRQGEHYPFLKDNKEKIEKILTFRIPYYVGPLARGNRDFAWLTRNS |
| | DQAIRPWNFEEVVDQASSAEDFINKMTNYDLYLPEEKVLPKHSLLYETFAVYNELTKVKFIAEGLR |
| | DYQFLDSGQKKQIVNQLFKEKRKVTEKDIIHYLHNVDGYDGIELKGIEKQFNASLATYHDLLKIIKD |
| | KEFMDDPKNEEILEKIIHTLTLFEDREMIKQRLEQYADIFDKSVLKKLSRRHYTGWGKLSAKLINGI |
| | RDKKTGKTILDYLIDDGYSNRNFMQLINDDSLSFKEKIKEAQRSGEGDDLREEVQNLAGSPAIKKGI |
| | LQSLKIVDELIEVMGYDPEHIVVEMARENQFTNQGRRNSQQRYKKLKNAIKNLDNNLNSKILKEYP |
| | TNNQALQNDRLFLYYIQNGKDMYTGEELDIDNLSQYDIDHIIPQAFIKDDSIDNRVLTSSAKNRGKS |
| | DDVPSIEIVKDRRNFWKQLLDSQLISQRKFDNLTKAERGGLTEADKAGFIKRQLVETRQITKHVAQI |
| | LDARFNTKRDENDKVIRDVKVITLKSNLVSQFRKDFQFYKVREINDYHHAHDAYLNAVVGTALIK |
| | KYPKLESEFVYGDYKVYDVRRMIAKSEQEIGKATAKRFFYSNILNFFKTEIKLANGEIRKRPLIETNE |
| | ETGEIVWNKEKDFATVRKVLSCPQVNIVKKTEVQTGGFSKESILPKRNSDKLIARKKDWDPKKYG |
| | GFDSPTVAYSVLVVAKVEKGKSKKLKSVKELLGITIMERSSFEKNPIDFLEAKGYKEVRKDLIVKLP |
| | KYSLFELENGRKRMLASAGELQKGNELALPSKYVNFLYLASHYEKLKGSPEDNEQKQLFVEQHKH |
| | YLDEIIEQISEFSKRVILADANLDKVLSAYNKHRDKPIREQAENIIHLFTLTNLGAPAAFKYFDTTIDR |
| | KRYTSTKEVLDATFIHQSITGLYETRIDLSQLGGD |
| 49 | MTKPYSIGLDIGTNSVGWAVITDSYKVPSKKMKVLGNTSKKYVKKNLLGALLFESGITAEGRRLK |
| | RTARRRYTRRRNRILYLQEIFSTEMAKIDESFFQRLDDSFLVPDDKRDSKYPIFGNLVKEKTYHDEF |
| | PTIYHLRKYLADSSKKADLRLVYLALAHMIKYRGHFLIEGDFNSRNNDIKKNFQDFLDSYNAIFESD |
| | LSLENSKQLEEIVKDKISKSAKKDRLLKLFPGEKNSGIFSEFLKLIVGNQADFRKYFNLDEKASLHES |
| | KESYDEDLETLLGHIGDDYSDVFLKAKKLYDAILLSGILTVTDNETEAPLSSAMIMRYKEHEEDLAL |
| | LKAYIRNISLETYNEVFKDDTKNGYAGYIDGKTNQEDFYVYLKKLLTGLEGADYFLEKINREDLLR |
| | KQRTFDNGSIPYQIHLQEMRAIIDKQAKFYPFLAKNKERIEKILTFRIPYYVGPLARGNSDFAWSIRK |
| | RNEKITPWNFEDVIDKESSAEAFINRMTSFDLYLPEEKVLPKHSLLYETFTVYNELTKVRFIAESMR |
| | DYQFLDSKQKKDIVRLYFKGKRKVTDKDIIEYLHAIDGYDGIELKGIEKQFNSSLSTYHDLLNIINDK |
| | EFLDDSSNEAIIEEIIHTLTIFEDREMIKQRLSKFENIFDKSVLKKLSRRHYTGWGKLSAKLINGIRDE |
| | KSGKTILDYLIDDGISNRNFMQLIHDDTLSFKKKIQKAQIIGDKDNIKQVVQSLPGSPAIKKGILQSIK |
| | IVDELVKVMGGRKPESIVVEMARENQYTNQGKSNSQQRLKRLEESLKELGSKILKENVPAKLSKID |
| | NNALQNERLYLYYLQNGKDMYTGDDLDIDRLSNYDIDHIIPQAFLKDNSIDNKVLVSSASNRGKSD |
| | DVPSLDVVKKRKGFWHQLLKSKLISQRKFDNLTKAERGGLSPEDKAGFIQRQLVETRQITKHVARI |
| | LDERFNNEVNDKGQRIRKVKIITLKSSLVSQFRKEFGLYKVREINDYHHAHDAYLNAVVATAILKK |
| | YPKLEPEFVYGDYQKYDVRKYLSTKDNKDLGKATAKLFFYSNLMNFFKKEVRLSDGTVIVRPVIE |
| | YSKDTGEIAWNKEKDFATVRKVLSCPQVNIVKKTEVQTGGFSKESILPKRNSDKLIARKKDWDPK |
| | KYGGFDSPTVAYSVLVVAKVEKGKSKKLKSVKELLGITIMERSSFEKNPIDFLEAKGYKEVRKDLI |
| | VKLPKYSLFELENGRKRMLASAGELQKGNELALPSKYVNFLYLASHYEKLKGSPEDNEQKQLFVE |
| | QHKHYLDEIIEQISEFSKRVILADANLDKVLSAYNKHRDKPIREQAENIIHLFTLTNLGAPAAFKYFD |
| | TTIDRKRYTSTKEVLDATFIHQSITGLYETRIDLSQLGGD |
| 50 | MTKPYSIGLDIGTNSVGWAVITDSYKVPSKKMKVLGNTSKKYVKKNLLGALLFESGITAEGRRLK |
| | RTARRRYTRRRNRILYLQEIFSTEMAKIDESFFQRLDDSFLVPDDKRDSKYPIFGNLVKEKTYHDEF |
| | PTIYHLRKYLADSSKKADLRLVYLALAHMIKYRGHFLIEGDFNSRNNDIKKNFQDFLDSYNAIFESD |
| | LSLENSKQLEEIVKDKISKSAKRERVLKLFPDEKSTGLFSEFLKLIVGNQADFKKHFDLEEKAPLQFS |
| | KDTYDEDLENLLGQIGDGFADLFLVAKKLYDAILLSGILTVTDPSTKAPLSASMIERYENHQKDLA |
| | ALKQFIQNNLQEKYDEVFSDQSKDGYAGYINGKTTQEAFYKYIKNLLSKLEGADYFLDKIEREDFL |
| | RKQRTFDNGSIPHQIHLQEMNAILRRQGEHYPFLKENKEKIEKILTFRIPYYVGPLARGNRDFAWLT |
| | RNSDQAIRPWNFEEIVDQASSAEDFINKMTNYDLYLPEEKVLPKHSLLYETFAVYNELTKVKFIAEG |
| | LRDYQFLDSGQKKQIVNQLFKEKRKVTEKDIIQFLHNVDGYDGIELKGIEKQFNASLSTYHDLLKII |
| | KDKEFMDDPKNEEILENVVHTLTIFEDREMIKQRLTQYASIFDEKVIKALSRRHYTGWGRLSAKLIN |
| | GIRDKQSQKTILDYLIDDDKINRNFMQLINDDGLSFKDIIQKAQVVGKTDDVKQVVQELPGSPAIKK |
| | GILQSIKIVDELVKVMGHAPESIVIEMARENQTTARGKKNSQQRYKRIEDALKNLAPELDGNILKEY |
| | PTDNQALQNDRLFLYYLQNGKDMYTGEELDINQLSNYDIDHIVPQAFIKDDSLDNRVLTSSKDNRG |
| | KSDNVPSLEVVQKRKAFWQQLLDSKLISERKENNLTKAERGGLDERDKVGFIKRQLVETRQITKHV |
| | AQILDARFNTEVDEKGKKIREVKIVTLKSNLVSNFRKEFGLYKVREINDYHHAHDAYLNAVVAKAI |
| | LKKYPKLEPEFVYGDYQKYDVKRYISRSKDPKEIEKATEKYFFYSNLLNFFKEEVHYADGTIIKREN |
| | IEYSKDTGEIAWNKEKDFATVRKVLSCPQVNIVKKTEVQTGGFSKESILPKRNSDKLIARKKDWDP |
| | KKYGGFDSPTVAYSVLVVAKVEKGKSKKLKSVKELLGITIMERSSFEKNPIDFLEAKGYKEVRKDL |
| | IVKLPKYSLFELENGRKRMLASAGELQKGNELALPSKYVNFLYLASHYEKLKGSPEDNEQKQLFVE |
| | QHKHYLDEIIEQISEFSKRVILADANLDKVLSAYNKHRDKPIREQAENIIHLFTLTNLGAPAAFKYFD |
| | TTIDRKRYTSTKEVLDATFIHQSITGLYETRIDLSQLGGD |
| 51 | MTKPYSIGLDIGTNSVGWAVITDSYKVPSKKMKVLGNTSKKYVKKNLLGALLFESGITAEGRRLK |
| | RTARRRYTRRRNRILYLQEIFSTEMAKIDESFFQRLDDSFLVPDDKRDSKYPIFGNLVEEKAYHDEF |
| | PTIYHLRKYLADSSKKADLRLVYLALAHMIKYRGHFLIEGDFNSRNNDIKKNFQDFLDSYNAIFESD |
| | LSLENSKQLEEIVKDKISKSAKKDRLLKLFPGEKNSGIFSEFLKLIVGNQADFRKYFNLDEKASLHES |
| | KESYDEDLETLLGHIGDDYSDVFLKAKKLYDAILLSGILTVTDNETEAPLSSAMIMRYKEHEEDLAL |
| | LKAYIRKISLETYNEVFKDDTKNGYAGYIDGKTNQEDFYVYLKKLLTGLEGADYFLEKINREDLLR |
| | KQRTFDNGSIPYQIHLQEMRAIIDKQAKFYPFLAKNKERIEKILTFRIPYYVGPLARGNSDFAWSIRK |
| | RNEKITPWNFEDVIDKESSAEAFINRMTSFDLYLPEEKVLPKHSLLYETFTVYNELTKVRFIAESMR |
| | DYQFLDSKQKKDIVRLYFKGKRKVTDKDIIDYLHAIDGYDGIELKGIEKQFNSSLATYHDLLNIIND |
| | KEFLDDSSNEAIIEEIIHTLTIFEDREMIKQRLSKFENIFDKSVLKKLSRRHYTGWGKLSAKLINGIRD |
| | EKSGKTILDYLIDDGVSNRNFMQLIHDDALSFKKKIQKAQIIGDQDNIKQVVQSLPGSPAIKKGILQS |
| | IKIVDELVKVMGRNPESIVVEMARENQYTNQGKSNSQQRLKRLEESLKELGSKILKENVPVKLSKI |
| | DNKALQNERLYLYYLQNGKDMYTGNDLDIDRLSNYDIDHIIPQAFLKDNSIDNKVLVSSASNRGKS |
| | DDVPSIDVVKKRKTFWHQLLKSKLISQRKFDNLTKAERGGLTEEDKAGFIHRQLVETRQITKHVAQ |
| | LLDSRFNTEKDDNNKAVRTVKIITLKSSLVSQFRKDFKLYKVREINDYHHAHDAYLNAVVATALL |
| | KKYPKLAPEFVYGQYEKYEKYKVIDKKSGEISKTKDTSKYGKATAKYFFYSNLMNFFKRVIRYSN |
| | GKVIIRPVVEYSKDTGEVAWNKEKDFATVRKVLSCPQVNIVKKTEVQTGGFSKESILPKRNSDKLI |
| | ARKKDWDPKKYGGFDSPTVAYSVLVVAKVEKGKSKKLKSVKELLGITIMERSSFEKNPIDFLEAK |
| | GYKEVRKDLIVKLPKYSLFELENGRKRMLASAGELQKGNELALPSKYVNFLYLASHYEKLKGSPE |
| | DNEQKQLFVEQHKHYLDEIIEQISEFSKRVILADANLDKVLSAYNKHRDKPIREQAENIIHLFTLTNL |
| | GAPAAFKYFDTTIDRKRYTSTKEVLDATFIHQSITGLYETRIDLSQLGGD |
| 52 | MTKPYSIGLDIGTNSVGWAVITDSYKVPSKKMKVLGNTSKKYVKKNLLGALLFESGITAEGRRLK |
| | RTARRRYTRRRNRILYLQEIFSTEMAKIDESFFQRLDDSFLVPDDKRDSKYPIFGNLVKEKTYHDEF |
| | PTIYHLRKYLADSSKKADLRLVYLALAHMIKYRGHFLIEGDFNSRNNDIKKNFQDFLDSYNAIFESD |
| | LSLENSEQLEEIVKDKISKSAKRERVLKLFPDEKSTGLFSEFLKLIVGNQADFKKHFDLEEKAPLQFS |
| | KDTYDEDLENLLGQIGDDFADLFLVAKKLYDAILLSGILTVTDPSTKAPLSASMIERYENHQKDLAT |
| | LKQFVRTNLPEKYDEVFSDQSKDGYAGYIDGKTTQEAFYKYIKNLLSKFEGADYFLDKIEREDFLR |
| | KQRTFDNGSIPHQIHLQEMNAIIRRQGEHYPFLKENKEKIEKILTFRIPYYVGPLARGNRDFAWLTR |
| | NSDQAIRPWNFEEIVDQASSAEDFINKMTNYDLYLPEEKVLPKHSLLYETFAVYNELTKVKFIAEGL |
| | RDYQFLDSGQKKQIVNQLFKEKRKVTEKDIIHYLHNVDGYDGIELKGIEKQFNASLSTYHDLLKIIK |
| | DKEFMDDPKNEEILENVVHTLTIFEDREMIKQRLAQYDSLFDEKVIKALTRRHYTGWGKLSAKLIN |
| | GICDKQTGKTILDYLIDDGEINRNFMQLIHDDGLSFKDIIQKAQVVGKTDDVKQVVQELPGSPAIKK |
| | GILQSIKIVDELVKVMGHNPENIVVEMARENQFTNQGRRNSQQRYKKIEDAIKNLDSNLNSKILKE |
| | YPTNNQALQNDRLFLYYLQNGKDMYTGEELDINQLSNYDIDHIVPQAFIKDDSLDNRVLTSSKDNR |
| | GKSDNVPSLEVVQKRKAFWQQLLDSKLISERKENNLTKAERERGGLNELDKVGFIKRQLVETRQIT |
| | KHVAQILDARFNTEVNEKGQRIRKVKIITLKSNLVSNFRKEFGLYKVREINDYHHAHDAYLNAVVA |
| | KAILKKYPKLEPEFVYGDYQKYDVKRYISRSKDPKEIEKATEKYFFYSNLLNFFKEEVHYADGTIIK |
| | RENIEYSKDTGEIAWNKEKDFATVRKVLSCPQVNIVKKTEVQTGGFSKESILPKRNSDKLIARKKD |
| | WDPKKYGGFDSPTVAYSVLVVAKVEKGKSKKLKSVKELLGITIMERSSFEKNPIDFLEAKGYKEVR |
| | KDLIVKLPKYSLFELENGRKRMLASAGELQKGNELALPSKYVNFLYLASHYEKLKGSPEDNEQKQ |
| | LFVEQHKHYLDEIIEQISEFSKRVILADANLDKVLSAYNKHRDKPIREQAENIIHLFTLTNLGAPAAF |
| | KYFDTTIDRKRYTSTKEVLDATFIHQSITGLYETRIDLSQLGGD |
| 53 | MTKPYSIGLDIGTNSVGWAVITDSYKVPSKKMKVLGNTSKKYVKKNLLGALLFESGITAEGRRLK |
| | RTARRRYTRRRNRILYLQEIFSTEMAKIDESFFQRLDDSFLVPDDKRDSKYPIFGNLVEEKTYHDEFP |
| | TIYHLRKYLADSSKKADLRLVYLALAHMIKYRGHFLIEGDFNSRNNDIKKNFQDFLDSYNAIFESDL |
| | SLENSKQLEEIVKDKISKSAKKDRLLKLFPGEKNSGIFSEFLKLIVGNQADFRKYFNLDEKASLHFSK |
| | ESYDEDLETLLGHIGDDYSDVFLKAKKLYDAILLSGILTVTDNETEAPLSSAMIMRYKEHEEDLALL |
| | KAYIRKISLETYNEVFKDDTKNGYAGYIDGKTNQEDFYVYLKKLLTGLEGADYFLEKINREDLLRK |
| | QRTFDNGSIPYQIHLQEMRAIIDKQAKFYPFLAKNKERIEKILTFRIPYYVGPLARGNSDFAWSIRKR |
| | NEKITPWNFEDVIDKESSAEAFINRMTSFDLYLPEEKVLPKHSLLYETFTVYNELTKVRFIAESMRD |
| | YQFLDSKQKKDIVRLYFKGKRKVTDKDIIEYLHAIDGYDGIELKGIEKQFNSSLSTYHDLLNIINDKE |
| | FLDDSSNEAIIEEIIHTLTIFEDREMIKQRLSKFENIFDKSVLKKLSRRHYTGWGKLSAKLINGIRDEK |
| | SGKTILDYLIDDGISNRNFMQLIHDDTLSFKKKIQKKQYIEDQDNIKQVVQSLPGSPAIKKGILQSIKI |
| | VDELVKVMGGRKPESIVVEMARENQYTNQGKSNSQQRLKRLEESLKELGSKILKENVPAKLSKID |
| | NNALQNERLYLYYLQNGKDMYTGDDLDIDRLSNYDIDHIIPQAFLKDNSIDNKVLVSSASNRGKSD |
| | DVPSLDVVKKRKTFWHQLLKSKLISQRKFDNLTKAERGGLTEDDKAGFIKRQLVETRQITKHVAQI |
| | LDARFNTKRDDNNKVIRDVKIITLKSSLVSQFRKEFKLYKVREINDYHHAHDAYLNAVVATAILKK |
| | YPKLEPEFVYGDYEKHELSDTKGDHKNFDLISKVKSDSSLGKATAKMFFYSNLMNFFKKEVRLAD |
| | GTVIVRPVIEYSKDTGEIAWNKEKDFATVRKVLSCPQVNIVKKTEVQTGGFSKESILPKRNSDKLIA |
| | RKKDWDPKKYGGFDSPTVAYSVLVVAKVEKGKSKKLKSVKELLGITIMERSSFEKNPIDFLEAKG |
| | YKEVRKDLIVKLPKYSLFELENGRKRMLASAGELQKGNELALPSKYVNFLYLASHYEKLKGSPED |
| | NEQKQLFVEQHKHYLDEIIEQISEFSKRVILADANLDKVLSAYNKHRDKPIREQAENIIHLFTLTNLG |
| | APAAFKYFDTTIDRKRYTSTKEVLDATFIHQSITGLYETRIDLSQLGGD |
| 54 | MTKPYSIGLDIGTNSVGWAVITDSYKVPSKKMKVLGNTSKKYVKKNLLGALLFESGITAEGRRLK |
| | RTARRRYTRRRNRILYLQEIFSTEMAKIDESFFQRLDDSFLVPEDKRGSKYPIFGNLAKEKTYHDEFP |
| | TIYHLRKYLADSQKKADLRLVYLALAHMIKYRGHFLIEGDFNSRNNDIKKNFQEFLDSYNAIFESD |
| | LSLENSKQLEEIVKDKISKSAKRERVLKLFPDEKSTGLFSEFLKLIVGNQADFKKHFDLEEKAPLQFS |
| | KDTYDEDLENLLGQIGDDFADLFLVAKKLYDAILLSGILTVTDPSTKAPLSASMIERYENHQKDLA |
| | QLKQFVRRNLPEKYAEVFSDQSKDGYAGYIDGKTTQEAFYKYIKNLLTKFEGAEYLLEKIKREDLL |
| | RKQRTFDNGSIPYQVHLQEMKAILQHQGEYYPFLKENKEKIQQILTFRIPYYVGPLARGNRDFAWL |
| | TRNSDQAIRPWNFEEIVDKASSAEDFINKMTNYDLYLPEEKVLPKHSLLYETFAVYNELTKVKFIAE |
| | GLRDYQFLDSGQKKQIVNQLFKEKRKVTEKDIIHYLHNVDGYDGIELKGIEKQFNASLSTYHDLLKI |
| | IKDKEFMDDPKNEEILENIVHTLTIFEDREMIKQRLAQYDSLFDEKVIKALIRRHYTGWGKLSAKLI |
| | NGIYDKQSKKTILDYLIDDGEINRNFMQLIHDDGLSFKEIIQKAQVFGKTDDVKQVVQELPGSPAIK |
| | KGILQSIKIVDELVKVMGHAPESIVIEMARENQTTARGKKNSQQRYKRIEDALKNLAPGLDSNILKE |
| | NPTDNIQLQNDRLFLYYLQNGKDMYTGEALDINQLSSYDIDHIIPQAFIKDDSLDNRVLTSSKDNRG |
| | KSDNVPSIEVVQKRKAFWQQLLDSKLISERKYNNLTKAERERGGLTELDKAGFIKRQLVETRQITK |
| | HVAQILDSRENTEVNDEGKRIRKVKIITLKSNLVSNFRKEFGLYKVREINDYHHAHDAYLNAVVAK |
| | AILKKYPKLEPEFVYGDYQKYDIKRYISRSKDPKEIEKATEKYFFYSNLLNFFKEEVHYADGTIIKRE |
| | NIEYSKDTGEIAWNKEKDFATVRKVLSCPQVNIVKKTEVQTGGFSKESILPKRNSDKLIARKKDWD |
| | PKKYGGFDSPTVAYSVLVVAKVEKGKSKKLKSVKELLGITIMERSSFEKNPIDFLEAKGYKEVRKD |
| | LIVKLPKYSLFELENGRKRMLASAGELQKGNELALPSKYVNFLYLASHYEKLKGSPEDNEQKQLFV |
| | EQHKHYLDEIIEQISEFSKRVILADANLDKVLSAYNKHRDKPIREQAENIIHLFTLTNLGAPAAFKYF |
| | DTTIDRKRYTSTKEVLDATFIHQSITGLYETRIDLSQLGGD |
| 55 | MNKPYSIGLDIGTNSVGWAVITDSYKVPSKKMKVLGNTSKKYVKKNLLGALLFESGITAEGRRLK |
| | RTARRRYTRRRNRILYLQEIFSTEMAKIDESFFQRLDDSFLVPDDKRDSKYPIFGNLVEEKTYHDEFP |
| | TIYHLRKYLADSSKKADLRLVYLALAHMIKYRGHFLIEGDFNSRNNDIKKNFQDFLDSYNAIFESDL |
| | SLENSKQLEEIVKDKISKSAKRERVLSLFPDEKSTGLFSEFLKLIVGNQADFKKHFDLEEKAPLQFSK |
| | DTYDEDLENLLGQIGDDFADLFLVAKKLYDAILLSGILTVTDPSTKAPLSASMIERYENHQKDLAAL |
| | KQFIQNNLQEKYDEVFSDQSKDGYAGYIDGKTTQEAFYKYIKNLLSKLEGADYFLDKIEREDFLRK |
| | QRTFDNGSIPHQIHLQEMNAIIHRQGEHYPFLKENREKIEKILTFRIPYYVGPLARGNRDFAWLTRNS |
| | DQAIRPWNFEEIVDQASSAEEFINKMTNYDLYLPEEKVLPKHSLLYETFAVYNELTKVKFIAEGLRD |
| | YQFLDSGQKKQIVNQLFKEKRKVTEKDIIHFLHNVDGYDGIELKGIEKQFNASLSTYHDLLKIIKDK |
| | EFMDDPKNEEILENIVHTLTIFEDREMIKQRLAQYDSLFDEKVIKALTRRHYTGWGKLSAKLINGIC |
| | DKQTGDTILDFLIDDDKINRNFMQLINDDGLSFKDIIQKAQVVGKTDDVKQVVQELPGSPAIKKGIL |
| | QSIKIVDELVKVMGHAPESIVIEMARENQTTARGKKNSQQRYKRIEDALKNLAPGLDSNILKEHPT |
| | DNIALQNDRLFLYYLQNGKDMYTGEPLDINQLSSYDIDHIVPQAFIKDDSIDNRVLTSSKDNRGKSD |
| | NVPSLEVVQKRKAFWQQLLDSKLISERKENNLTKAERGGLDERDKVGFIKRQLVETRQITKHVAQI |
| | LDARFNTEVDENGKRIRKVKIVTLKSNLVSNFRKEFGLYKVREINDYHHAHDAYLNAVVAKAILK |
| | KYPKLEPEFVYGDYQKYDVKRYISRSKDPKDIEKATQKYFFYSNLLNFFKEEVHYADGTIIKRENIE |
| | YSKDTGEIAWNKEKDFATVRKVLSCPQVNIVKKTEVQTGGFSKESILPKRNSDKLIARKKDWDPK |
| | KYGGFDSPTVAYSVLVVAKVEKGKSKKLKSVKELLGITIMERSSFEKNPIDFLEAKGYKEVRKDLI |
| | VKLPKYSLFELENGRKRMLASAGELQKGNELALPSKYVNFLYLASHYEKLKGSPEDNEQKQLFVE |
| | QHKHYLDEIIEQISEFSKRVILADANLDKVLSAYNKHRDKPIREQAENIIHLFTLTNLGAPAAFKYFD |
| | TTIDRKRYTSTKEVLDATFIHQSITGLYETRIDLSQLGGD |
| 56 | MKKSYSIGLDIGTNSVGWSVITDDYKVPAKKMKVLGNTDKKYIKKNLLGALLFDSGETAEATRLK |
| | RTARRRYTRRKNRLRYLQEIFAKEMAKVDESFFQRLEESFLTDDDKNFDSHPIFGNKAEEDAYHDE |
| | FPTIYHLRQYLADSPQKADLRLVYLALAHMIKFRGHFLIEGQLNAENTDVQTLFNDFVEVYDKTVE |
| | ESHLSEITVDALSILTEKVSKSRRLENLVKCYPTEKKNTLFGNLIALSLGLQPNFKTNFKLSEDAKLQ |
| | FSKDTYEEDLEELLGKIGDEYADLFASAKNLYDAILLSGILTVDDNTTKAPLSASMVKRYAEHHED |
| | LEKLKEFVKANKPELYHDIFKDETKNGYAGYIENGVKQDEFYKYLKNTLSKIAGSDYFLDKIERED |
| | FLRKQRTFDNGSIPHQIHLQEMHAILRRQGEYYPFLKENQAKIEKILTFRIPYYVGPLARKNSRFAW |
| | AEYHSDEKITPWNFEEIVDKEESATAFIHRMTNNDLYLPEEKVLPKHSLIYEEFTVYNELTKVKYVT |
| | DQGKAYYFDANMKQEIFDGVFKEHRKVTKDKLMNFLEKEFDEFRIVDLTGLDKENKSFNASLGTY |
| | HDLKKILDKSFLDDKTNETIIEDIIQTLTLFEDRDMIRQRLQKYSDIFTPQQLKKLERRHYTGWGRLS |
| | YKLINGIRNKENGKTILDYLIDDGYANRNFMQLINDDTLPFKQIIQEAQIIGDIDDIETVVHDLPGSPA |
| | IKKGILQSVKIVDELVKVMGHNPDNIVIEMARENQTTNRGRSQSQQRLKKLQESLKELGSNILNEEK |
| | PSYVEDKVENSHLQNDKLFLYYIQNGKDMYTGEELDIDHLSDYDIDHIIPQAFIKDNSIDNRVLTSS |
| | AKNRGKSDDVPSLDIVHDRKADWIRLYKSGLISKRKFDNLTKAEHGGLTEDDKAGFIKRQLVETR |
| | QITKHVAQILDSRENTERDENDKVIRDVKVITLKSNLVSQFRKDFELHKVREINDYHHAHDAYLNA |
| | VIGNALLVKYPQLEPEFVYGKFKHFHGHKENKATAKKFFYSNIMALFKKDVEYSDDTGEMVWNK |
| | EKDFATVRKVLSCPQVNIVKKTEVQTGGFSKESILPKRNSDKLIARKKDWDPKKYGGFDSPTVAYS |
| | VLVVAKVEKGKSKKLKSVKELLGITIMERSSFEKNPIDFLEAKGYKEVRKDLIVKLPKYSLFELENG |
| | RKRMLASAGELQKGNELALPSKYVNFLYLASHYEKLKGSPEDNEQKQLFVEQHKHYLDEIIEQISE |
| | FSKRVILADANLDKVLSAYNKHRDKPIREQAENIIHLFTLTNLGAPAAFKYFDTTIDRKRYTSTKEV |
| | LDATFIHQSITGLYETRIDLSQLGGD |
| 57 | MTKPYSIGLDIGTNSVGWAVITDSYKVPSKKMKVLGNTSKKYVKKNLLGALLFESGITAEGRRLK |
| | RTARRRYTRRRNRILYLQEIFSTEMAKVDESFFQRLDDSFLVPDDKRDSKYPIFGNLVEEKTYHDEF |
| | PTIYHLRKYLADSSKKADLRLVYLALAHMIKYRGHFLIEGDFDTRNNNIKGIYQKFLDSYNAIFESD |
| | LSLENSKQLEEIVKDKISKSAKRERVLKLFPDEKSTGLFSEFLKLIVGNQADFKKHFELEEKAPLQFS |
| | KDTYDEDLENLLGQIGDDFADLFLVAKKLYDAILLSGILTVTDPSTKAPLSASMIERYENHQKDLA |
| | ALKQFIQNNLQEKYDEVFSDQSKDGYAGYIDGKTTQEAFYKYIKNLLSKLEGADYFLDKIEREDFL |
| | RKQRTFDNGSIPHQIHLQEMNAIIRRQGEHYPFLQENKEKIEKILTFRIPYYVGPLARGNRDFAWLTR |
| | NSDQAIRPWNFEEVVDKASSAEDFINKMTNYDLYLPEEKVLPKHSLLYETFAVYNELTKVKFIAEG |
| | LRDYQFLDSGQKKQIVNQLFKEKRKVTEKDIIHYLHNVDGYDGIELKGIEKQFNASLSTYHDLLKII |
| | KDKEFMDDPKNEEILENIVHTLTIFEDREMIKQRLAQYDSLFDEKVIKALTRRHYTGWGKLSAKLIN |
| | GIRDKQTNKTILDYLIDDGKINRNFMQLIHDDGLSFKEIIQKAQVVGKTDDVKQVVQELPGSPAIKK |
| | GILQSIKIVDELVKVMGHAPESIVIEMARENQTTNQGRRNSQQRYKLIEDGVKNLAKDLDGNILKE |
| | YPTDNQALQNDRLFLYYLQNGKDMYTGEALDINQLSNYDIDHIIPQAFIKDDSLDNRVLTSSKDNR |
| | GKSDNVPSIEVVQKRKAFWQQLLDSKLISERKENNLTKAERGGLDERDKVGFIKRQLVETRQITKH |
| | VAQILDSRFNTEVDETGKRIRKVKIITLKSNLVSNFRKEFGLYKVREINDYHHAHDAYLNAVVAKA |
| | ILKKYPKLEPEFVYGDYQKYDIKRYISRSKDPKDIEKATEKYFFYSNLLNFFKEEVHYADGTILKKD |
| | SVEYSKDTGEIAWNKKKDFAIVRKVLSCPQVNIVKKTEVQTGGFSKESILPKRNSDKLIARKKDWD |
| | PKKYGGFDSPTVAYSVLVVAKVEKGKSKKLKSVKELLGITIMERSSFEKNPIDFLEAKGYKEVRKD |
| | LIVKLPKYSLFELENGRKRMLASAGELQKGNELALPSKYVNFLYLASHYEKLKGSPEDNEQKQLFV |
| | EQHKHYLDEIIEQISEFSKRVILADANLDKVLSAYNKHRDKPIREQAENIIHLFTLTNLGAPAAFKYF |
| | DTTIDRKRYTSTKEVLDATFIHQSITGLYETRIDLSQLGGD |
| 58 | MKKSYSIGLDIGTNSVGWAVVDDDYKVPAKKMRVFGDTDRSHIKKNLLGTLLFDSGETAESRRLK |
| | RTARRRYTRRRNRILYLQEIFAEEMNKVDDSFFHRLDESFLTDDDKTFDSHPIFGNKAEEDAYHQK |
| | FPTIYHLRKHLADSTEKADLRLVYLALAHMIKFRGHFLIEGELNAENTDVQKLFADFVGVYDRTFD |
| | DSHLSEITVDASSILTEKISKSRRLENLIKHYPTEKKNTLFGNLVALALGLQPNFKTNFKLSEDAKLQ |
| | FSKDTYEEDLEELLGKIGDDYADLFTSAKNLYDAILLSGILTVDDESTKAPLSASMIKRYAEHHEDL |
| | EKLKEFIKANKPELYHDIFKDETKNGYAGYIENGVKQDEFYKYLKETLSKIAGSDYFLDKIEREDFL |
| | RKQRTFDNGSIPHQIHLQEMHAILRRQGDYYPFLKENQAKIEKILTFRIPYYVGPLARKDSRFAWAN |
| | YHSDEPITPWNFDEVVDKEKSAEKFITRMTLNDLYLPEEKVLPKHSHVYETYAVYNELTKIKYVNE |
| | QGKSFFFDANMKQEIFEHVFKENRKVTKEKFLNYLNKEFPEYRIQDLIGLDKENKSFNASLGTYHD |
| | LKKILDKSFLDDKENETIIEDIIQTLTLFEDRDMIRQRLQKYSDIFTPQQLKKLERRHYTGWGRLSYK |
| | LINGIRNKENNKTILDYLIDDGYANRNFMQLINDDTLPFKQIIQKSQVVGDIDDIKAVVHDLPGSPAI |
| | KKGILQSVKIVDELVKVMGHNPDNIVIEMARENQTTNRGRSQSQQRLKKLQDSLKELGSNILNEEK |
| | PSYIEDKVENSHLQNDKLFLYYIQNGKDMYTGEELDIDHLSDYDIDHIIPQAFIKDDSIDNRVLTSSA |
| | KNRGKSDDVPSLDIVHDRKADWIRLYKSGLISKRKFDNLTKAERGGLTEADKAGFIKRQLVETRQI |
| | TKHVAQILDARFNTERDENDKVIRDVKVITLKSNLVSQFRKDFQFYKVREINDYHHAHDAYLNAV |
| | VGTALLKKYPKLAPEFVYGEYKKYDIRKFITNSSDKATAKYFFYSNLLNFFKTKVRYADGTVIVRP |
| | VIEYSKDTGEIAWNKEKDFATVRKVLSCPQVNIVKKTEVQTGGFSKESILPKRNSDKLIARKKDWD |
| | PKKYGGFDSPTVAYSVLVVAKVEKGKSKKLKSVKELLGITIMERSSFEKNPIDFLEAKGYKEVRKD |
| | LIVKLPKYSLFELENGRKRMLASAGELQKGNELALPSKYVNFLYLASHYEKLKGSPEDNEQKQLFV |
| | EQHKHYLDEIIEQISEFSKRVILADANLDKVLSAYNKHRDKPIREQAENIIHLFTLTNLGAPAAFKYF |
| | DTTIDRKRYTSTKEVLDATFIHQSITGLYETRIDLSQLGGD |
| 59 | MTKPYSIGLDIGTNSVGWAVITDSYKVPSKKMKVLGNTSKKYVKKNLLGALLFESGITAEGRRLK |
| | RTARRRYTRRRNRILYLQEIFSTEMAKIDESFFQRLDDSFLVPDDKRDSKYPIFGNLVKEKTYHDEF |
| | PTIYHLRKYLADSSKKADLRLVYLALAHMIKYRGHFLIEGDFNSRNNDIKKNFQEFLDSYNAIFESD |
| | LSLENSKQLEEIVKDKISKSAKRERVLKLFPDEKSTGLFSEFLKLIVGNQADFKKHFDLEEKAPLQFS |
| | KDTYDEDLENLLGQIGDDFADLFLVAKKLYDAILLSGILTVTDPSTKAPLSASMIERYENHQKDLA |
| | ALKQFIKANLPEKYDEVFSDQSKDGYAGYIDGKTTQEAFYKYIKNLLSKFEGADYFLEKIEREDFLR |
| | KQRTFDNGSIPHQIHLQEMNAILHRQGDYYPFLKENKEKIQQILTFRIPYYVGPLARGNRDFAWLTR |
| | NSDQAIRPWNFEEVVDKASSAEDFINKMTNYDLYLPEEKVLPKHSLLYETFAVYNELTKVKFIAEG |
| | LRDYQFLDSGQKKQIVNQLFKEKRKVTEKDIIHYLHNVDGYDGIELKGIEKQFNASLSTYHDLLKII |
| | KDKEFMDDPKNEEILENIVHTLTIFEDREMIKQRLAQYDSLFDEKVIKALTRRHYTGWGKLSAKLIN |
| | GICDKQTGDTILDFLIDDDKINRNFMQLINDDGLSFKEIIQKAQVVGKTDDVKQVVQELPGSPAIKK |
| | GILQSIKIVDELVKVMGHAPESIVIEMARENQTTNQGRRNSQQRYKKIEEAIKNLDSNLNSKILKEYP |
| | TNNQALQNDRLFLYYLQNGKDMYTGEELDINQLSNYDIDHIVPQAFIKDDSLDNRVLTSSKDNRG |
| | KSDNVPSLEVVQKRKGFWQQLLDSKLISERKENNLTKAERGGLDERDKVGFIKRQLVETRQITKHV |
| | AQILDARFNKEVNDKKQRIRKVKIITLKSNLVSNFRKEFGLYKVREINDYHHAHDAYLNAVVAKAI |
| | LKKYPKLEPEFVYGDYQKYDIKRYISRSKDPKDVEKATEKYFFYSNLLNFFKEEVHYADGTIIKREN |
| | IEYSKDTGEIAWNKEKDFATVRKVLSCPQVNIVKKTEVQTGGFSKESILPKRNSDKLIARKKDWDP |
| | KKYGGFDSPTVAYSVLVVAKVEKGKSKKLKSVKELLGITIMERSSFEKNPIDFLEAKGYKEVRKDL |
| | IVKLPKYSLFELENGRKRMLASAGELQKGNELALPSKYVNFLYLASHYEKLKGSPEDNEQKQLFVE |
| | QHKHYLDEIIEQISEFSKRVILADANLDKVLSAYNKHRDKPIREQAENIIHLFTLTNLGAPAAFKYFD |
| | TTIDRKRYTSTKEVLDATFIHQSITGLYETRIDLSQLGGD |
| 60 | MTKPYSIGLDIGTNSVGWAVITDSYKVPSKKMKVLGNTSKKYVKKNLLGALLFESGITAEGRRLK |
| | RTARRRYTRRRNRILYLQEIFSTEMSKIDESFFQRLDDSFLVPDDKRDSKYPIFGNLAQEKTYHDEFP |
| | TIYHLRKYLADSSKKADLRLVYLALAHMIKYRGHFLIEGDENSKNNDIKKNFQEFLDSYNAIFESDL |
| | SLENSKQLEEIVKDKISKSAKRERVLKLFPDEKSTGLFSEFLKLIVGNQADFKKHFDLEEKAPLQFSK |
| | DTYDEDLENLLGQIGDDFADLFLVAKKLYDAILLSGILTVTDPSTKAPLSASMIERYENHQKDLAAL |
| | KQFIQNNLQEKYDEVFSDQSKDGYAGYIDGKTTQEAFYKYIKNLLSKFEGADYFLEKIEREDFLRK |
| | QRTFDNGTIPYQVHLEEMKAILHRQGDYYPFLKENKEKIQQLLTFRIPYYVGPLARGNRDFAWLTR |
| | NSDQAIRPWNFEEVVDKARSAEDFINKMTNYDLYLPEEKVLPKHSLLYETFAVYNELTKVKFIAEG |
| | LRDYQFLDSGQKKQIVNQLFKEKRKVTEKDIIHYLHTVDGYDGIELKGIEKQFNASLSTYHDLLKII |
| | KDKEFMDDPKNEEILENIVHTLTIFEDREMIKQRLTQYASIFDEKVIKALSRRHYTGWGKLSAKLIN |
| | GIRDKQSRKTILDYLIDDGEINRNFMQLIHDDGLSFKDIIQKAQVVGKTDDVKQVVQELPGSPAIKK |
| | GILQSIKLVDELVKVMGHAPESIVIEMARENQTTARGKKNSQQRYKRIEDALKNLAPGLDSNILKE |
| | HPTDNIQLQNDRLFLYYLQNGKDMYTGKSLDINQLSNYDIDHIVPQAFIKDDSLDNRVLTSSKDNR |
| | GKSDNVPSLEVVQKRKAFWQQLLDSKLISERKENNLTKAERGGLDERDKVGFIKRQLVETRQITKH |
| | VAQILDARFNTEVNEKNQKIRKVKIVTLKSNLVSNFRKEFGLYKVREINDYHHAHDAYLNAVVAK |
| | AILKKYPKLEPEFVYGDYQKYDVKRYISRSKDPKDIEKATEKYFFYSNLLNFFKEEVHYADGTIIKR |
| | ENIEYSKDTGEIAWNKEKDFATVRKVLSCPQVNIVKKTEVQTGGFSKESILPKRNSDKLIARKKDW |
| | DPKKYGGFDSPTVAYSVLVVAKVEKGKSKKLKSVKELLGITIMERSSFEKNPIDFLEAKGYKEVRK |
| | DLIVKLPKYSLFELENGRKRMLASAGELQKGNELALPSKYVNFLYLASHYEKLKGSPEDNEQKQLF |
| | VEQHKHYLDEIIEQISEFSKRVILADANLDKVLSAYNKHRDKPIREQAENIIHLFTLTNLGAPAAFKY |
| | FDTTIDRKRYTSTKEVLDATFIHQSITGLYETRIDLSQLGGD |
| 61 | MNKPYSIGLDIGTNSVGWAVITDSYKVPSKKMKVLGNTSKKYVKKNLLGALLFESGITAEGRRLK |
| | RTARRRYTRRRNRILYLQEIFSTEMAKIDESFFQRLDDSFLVPDDKRDSKYPIFGNLVEEKVYHDEF |
| | PTIYHLRKYLADSSKKADLRLVYLALAHMIKYRGHFLIEGDFNSRNNDIKKNFQDFLDSYNAIFESD |
| | LSLENSAQVEEIVKDKISKSAKRERVLKLFPDEKSTGLFSEFLKLIVGNQADFKKHFDLEEKAPLQFS |
| | KDTYDEDLENLLGQIGDDFADLFLVAKKLYDAILLSGILTVTDPSTKAPLSASMIERYENHQNDLSA |
| | LKQFIRRNIPEKYDEVFSDQSKDGYAGYIDGKTTQEAFYKYIKNTLSKIEGADYFLDKIEREDFLRK |
| | QRTFDNGSIPHQIHLQEMNAILHHQGEYYPFLQENKEKIEKILTFRIPYYVGPLARGNRDFAWLTRN |
| | SDQAIRPWNFEEVVDKASSAEDFINKMTNYDLYLPEEKVLPKHSLLYETFAVYNELTKVKFIAEGL |
| | RDYQFLDSGQKKQIVNQLFKEKRKVTEKDIIHFLHNVDGYDGIELKGIEKQFNASLSTYHDLLKIIK |
| | DKEFMDDPKNEEILENIVHTLTIFEDREMIKQRLAQYDSLFDEKVIKALTRRHYTGWGKLSAKLING |
| | ICDKQTGDTILDFLIDDDKINRNFMQLINDDGLSFKDIIQKAQVVGKTDDVKQVVQELPGSPAIKKG |
| | ILQSIKLVDELVKVMGHAPESIVIEMARENQTTARGKKNSQQRYKRIEDALKNLAPGLDSNILKENP |
| | TDNIQLQNDRLFLYYLQNGKDMYTGEALDINQLSNYDIDHIVPQAFIKDDSLDNRVLTSSKDNRGK |
| | SDNVPSLEVVQKRKAFWQQLLDSKLISEHKFNNLTKAERGGLDERDKVGFIRRQLVETRQITKHVA |
| | QILDARFNTEVNEKNQKIRKVKIITLKSNLVSNFRKEFGLYKVREINDYHHAHDAYLNAVVAKAIL |
| | KKYPKLEPEFVYGDYQKYDLKKYIPKSKSHGTIEKATQKYFFYSNLLNFFKEEVHYADGTIIKRENI |
| | EYSKDTGEIAWNKEKDFATVRKVLSCPQVNIVKKTEVQTGGFSKESILPKRNSDKLIARKKDWDPK |
| | KYGGFDSPTVAYSVLVVAKVEKGKSKKLKSVKELLGITIMERSSFEKNPIDFLEAKGYKEVRKDLI |
| | VKLPKYSLFELENGRKRMLASAGELQKGNELALPSKYVNFLYLASHYEKLKGSPEDNEQKQLFVE |
| | QHKHYLDEIIEQISEFSKRVILADANLDKVLSAYNKHRDKPIREQAENIIHLFTLTNLGAPAAFKYFD |
| | TTIDRKRYTSTKEVLDATFIHQSITGLYETRIDLSQLGGD |
| 62 | MTKPYSIGLDIGTNSVGWAVITDSYKVPSKKMKVLGNTSKKYVKKNLLGALLFESGITAEGRRLK |
| | RTARRRYTRRRNRILYLQEIFSTEMAKIDESFFQRLDDSFLVPDDKRDSKYPIFGNLVKEKTYHDEF |
| | PTIYHLRKYLADSSKKADLRLVYLALAHMIKYRGHFLIEGDFNSRNNDIKKNFQNFLDSYNAIFESD |
| | LSLENSKQLEEIVKDKISKSAKRERVLKLFPDEKSTGLFSEFLKLIVGNQADFKKHFDLEEKAPLQFS |
| | KDTYDEDLENLLGQIGDDFADLFLVAKKLYDAILLSGILTVTDPSTKAPLSASMIERYENHQKDLA |
| | QLKQFVKTNLPEKYDEVFSDQSKDGYAGYIDGKTTEEAFYKYIKNILNNVKGADYFLDKIEREDFL |
| | RKQRTFDNGSIPHQIHLQEMNAILRRQGEHYPFLKENKEKIEKILTFRIPYYVGPLARGNRDFAWLT |
| | RNSDQAIRPWNFEEIVDQASSAEDFINKMTNYDLYLPEEKVLPKHSLLYETFAVYNELTKVKFIAEG |
| | LRDYQFLDSGQKKQIVNQLFKEKRKVTEKDIIHYLHNVDGYDGIELKGIEKQFNASLSTYHDLLKII |
| | KDKEFMDDPKNEEILENIVHTLTIFEDREMIKQRLAQYDSLFDEKVIKALTRRHYTGWGKLSAKLIN |
| | GICDKQTGDTILDFLIDDDKINRNFMQLINDDGLSFKEIIQKAQVVGENDNLKQVVQNLPGSPAIKK |
| | GILQSIKIVDELVKVMGGDPEHIVVEMARENQFTNQGRRNSQQRYKKIENAIKNLDSSLNSKILKEY |
| | PTNNQALQNDRLFLYYLQNGKDMYTGEPLDINQLSNYDIDHIVPQAFIKDDSLDNRVLTSSKDNRG |
| | KSDNVPSIEIVEKMKGFWQQLLDSKLISERKENNLTKAERGGLDERDKVGFIKRQLVETRQITKHV |
| | AQILDARFNTEVDENNQKIRTVKIITLKSNLVSNFRKEFGLYKVREINDYHHAHDAYLNAVVAKAI |
| | LKKYPKLEPEFVYGDYQKYDLKRYLSKSGNPKAIDKATQKYFFYSNLLNFFKEEVHYADGTIVKR |
| | ENIEYSKDTGEIAWNKEKDFATVRKVLSCPQVNIVKKTEVQTGGFSKESILPKRNSDKLIARKKDW |
| | DPKKYGGFDSPTVAYSVLVVAKVEKGKSKKLKSVKELLGITIMERSSFEKNPIDFLEAKGYKEVRK |
| | DLIVKLPKYSLFELENGRKRMLASAGELQKGNELALPSKYVNFLYLASHYEKLKGSPEDNEQKQLF |
| | VEQHKHYLDEIIEQISEFSKRVILADANLDKVLSAYNKHRDKPIREQAENIIHLFTLTNLGAPAAFKY |
| | FDTTIDRKRYTSTKEVLDATFIHQSITGLYETRIDLSQLGGD |
| 63 | MTKPYSIGLDIGTNSVGWAVITDSYKVPSKKMKVLGNTSKKYVKKNLLGALLFESGITAEGRRLK |
| | RTARRRYTRRRNRILYLQEIFSTEMAKIDESFFQRLDDSFLVPDDKRDSKYPIFGNLVEEKTYHDEFP |
| | TIYHLRKYLADSSKKADLRLVYLALAHMIKYRGHFLIEGDFNSRNNDIKKNFQEFLDSYNAIFESDL |
| | SLENSAQVEEIVKDKISKSAKRERVLKLFPDEKSTGLFSEFLKLIVGNQADFKKHFDLEEKAPLQFS |
| | KDTYDEDLENLLGQIGDGFADLFLVAKKLYDAILLSGILTVTDPSTKAPLSASMIERYENHQKDLA |
| | ALKQFVKANLPEKYDEVFSDQSKDGYAGYIDGKTTQEAFYKYIKNLLSKLEGADYFLDKIEREDFL |
| | RKQRTFDNGSIPHQIHLQEMNAILRRQGEYYPFLKENKEKIEKILTFRIPYYVGPLARGNRDFAWLT |
| | RNSDQAIRPWNFEEVVDQASSAEDFINKMTNYDLYLPEEKVLPKHSLLYETFAVYNELTKVKFIAE |
| | GLRDYQFLDSGQKKQIVNQLFKEKRKVTEKDIIHFLHNVDGYDGIELKGIEKQFNASLSTYHDLLKI |
| | IKDKEFMDDPKNEEILENIVHTLTIFEDREMIKQRLAQYDSLFDEKVIKALARRHYTGWGKLSAKLI |
| | NGIHDKQSQKTILDYLIDDDKINRNFMQLINDDGLSFKDIIQKAQVVGKTDDVKQVVQELPGSPAIK |
| | KGILQSIKIVDELVKVMGHAPESIVIEMARENQTTARGKKNSQQRYKRIEDAIKGLSSDLNHKILKE |
| | HPTDNQALQNDRLFLYYLQNGKDMYTGEELDINQLSNYDIDHIIPQAFIKDDSLDNRVLTSSKDNR |
| | GKSDNVPSIEVVQKRKAFWQQLLDSKLISERKENNLTKAERGGLDEKDKVGFIRRQLVETRQITKH |
| | VAQILDARFNTEVDDKGKRIRKVKIVTLKSNLVSNFRKEFGLYKVREINDYHHAHDAYLNAVVAK |
| | AILKKYPKLEPEFVYGDYQKYDVKRYISRSKEPKDIEKATEKYFFYSNLLNFFKEEVHYADGTIIKR |
| | ENIEYSNDTGEIAWNKEKDFATVRKVLSCPQVNIVKKTEVQTGGFSKESILPKRNSDKLIARKKDW |
| | DPKKYGGFDSPTVAYSVLVVAKVEKGKSKKLKSVKELLGITIMERSSFEKNPIDFLEAKGYKEVRK |
| | DLIVKLPKYSLFELENGRKRMLASAGELQKGNELALPSKYVNFLYLASHYEKLKGSPEDNEQKQLF |
| | VEQHKHYLDEIIEQISEFSKRVILADANLDKVLSAYNKHRDKPIREQAENIIHLFTLTNLGAPAAFKY |
| | FDTTIDRKRYTSTKEVLDATFIHQSITGLYETRIDLSQLGGD |
| 64 | MTKPYSIGLDIGTNSVGWAVITDSYKVPSKKMKVLGNTSKKYVKKNLLGALLFESGITAEGRRLK |
| | RTARRRYTRRRNRILYLQEIFSTEMNKIDDAFFQRLDDSFLVPDDKRDSKYPIFGNLVKEKTYHDEF |
| | PTIYHLRKYLADSSKKADLRLVYLALAHMIKYRGHFLIEGDFNSRNNDIKKNFQDFLDSYNAIFESD |
| | LSLENSKQLEEIVKDKISKSAKKDRLLKLFPGEKNSGIFSEFLKLIVGNQADFRKYFNLDEKASLHES |
| | KESYDEDLETLLGHIGDDYSDVFLKAKKLYDAILLSGILTVTDNETEAPLSSAMIMRYKEHEEDLAL |
| | LKAYIRKISLETYNEVFKDDTKNGYAGYIDGKTNQEDFYVYLKKLLTGLEGADYFLEKINREDLLR |
| | KQRTFDNGSIPYQIHLQEMRAIIDKQAKFYPFLAKNKERIEKILTFRIPYYVGPLARGNSDFAWSIRK |
| | RNEKITPWNFEDVIDKESSAEAFINRMTSFDLYLPEEKVLPKHSLLYETFTVYNELTKVRFIAESMR |
| | DYQFLDSKQKKDIVRLYFKGKRKVTDKDIIEYLHAIDGYDGIELKGIEKQFNSSLATYHDLLNIIND |
| | KEFLDDSSNEAIIEEIIHTLTIFEDREMIKQRLSKFENIFDKSVLKKLSRRHYTGWGKLSAKLINGIRD |
| | EKSGKTILDYLIDDGISNRNFMQLIHDDTLSFKKKIQKAQIIGDQDNIKQVVQSLPGSPAIKKGILQSI |
| | KIVDELVKVMGRNPESIVVEMARENQYTNQGKSNSQQRLKRLEESLKELGSKILKENVPAKLSKID |
| | NNALQNERLYLYYLQNGKDMYTGKDLDIDRLSNYDIDHIIPQAFLKDNSIDNKVLVSSASNRGKSD |
| | DVPSLEVVKKRKTFWHQLLKSKLISQRKFDNLTKAERGGLTENDKAGFIKRQLVETRQITKHVAQI |
| | LDARFNTEKDDNNKAIREVKIITLKSSLVSQFRKDFQLYKVREINDYHHAHDAYLNAVVATAILKK |
| | YPKLEPEFVYGEYKKYDVRKFVAKDDKSLGKATAKMFFYSNLMNFFKKEVRFADGTVIVRPLIEY |
| | SKDTGEIAWNKEKDFATVRKVLSCPQVNIVKKTEVQTGGFSKESILPKRNSDKLIARKKDWDPKK |
| | YGGFDSPTVAYSVLVVAKVEKGKSKKLKSVKELLGITIMERSSFEKNPIDFLEAKGYKEVRKDLIV |
| | KLPKYSLFELENGRKRMLASAGELQKGNELALPSKYVNFLYLASHYEKLKGSPEDNEQKQLFVEQ |
| | HKHYLDEIIEQISEFSKRVILADANLDKVLSAYNKHRDKPIREQAENIIHLFTLTNLGAPAAFKYFDT |
| | TIDRKRYTSTKEVLDATFIHQSITGLYETRIDLSQLGGD |
| 65 | MTKPYSIGLDIGTNSVGWAVITDSYKVPSKKMKVLGNTSKKYVKKNLLGALLFESGITAEGRRLK |
| | RTARRRYTRRRNRILYLQEIFSTEMAKIDESFFQRLDDSFLVPDDKRDSKYPIFGNLVKEKTYHDEF |
| | PTIYHLRKYLADSSKKADLRLVYLALAHMIKYRGHFLIEGDFNSRNNDIKKNFQDFLDSYNAIFESD |
| | LSLENSKQLEEIVKDKISKSAKKDRLLKLFPGEKNSGIFSEFLKLIVGNQADFRKYFNLDEKASLHES |
| | KESYDEDLETLLGHIGDDYSDVFLKAKKLYDAILLSGILTVTDNETEAPLSSAMIMRYKEHEEDLAL |
| | LKAYIRKISLETYNEVFKDDTKNGYAGYIDGKTNQEDFYVYLKKLLTGLEGADYFLEKINREDLLR |
| | KQRTFDNGSIPYQIHLQEMRAIIDKQAKFYPFLAKNKERIEKILTFRIPYYVGPLARGNSDFAWSIRK |
| | RNEKITPWNFEDVIDKESSAEAFINRMTSFDLYLPEEKVLPKHSLLYETFTVYNELTKVRFIAESMR |
| | DYQFLDSKQKKDIVRLYFKGKRKVTDKDIIDYLHAIDGYDGIELKGIEKQFNSSLSTYHDLLNIIND |
| | KEFLDDSSNEAIIEEIIHTLTIFEDREMIKQRLSKFENIFDKSVLKKLSRRHYTGWGKLSAKLINGIRD |
| | EKSGKTILDYLIDDGISNRNFMQLIHDDTLSFKKKIQKEQYIEDQDNIKQVVQSLPGSPAIKKGILQSI |
| | KIVDELVKVMGGRKPESIVVEMARENQYTNQGKSNSQQRLKRLEESLKELGSKILKENVPAKLSKI |
| | DNNALQNERLYLYYLQNGKDMYTGEDLDIDRLSNYDIDHIIPQAFLKDNSIDNKVLVSSASNRGKS |
| | DDVPSLDVVKKRKGFWHQLLKSQLISQRKFDNLTKAERGGLTEDDKAGFIKRQLVETRQITKHVA |
| | QILDARFNTEPDENGKRIRKVKIITLKSSLVSQFRKEFGLYKVREINDYHHAHDAYLNAVVATAILK |
| | KYPKLEPEFVYGDYQKYDIRKYLSTKDNKDLGKATAKMFFYSNLMNFFKKEVRLSDGTVVVRPVI |
| | EYSKDTGEIAWNKEKDFATVRKVLSCPQVNIVKKTEVQTGGFSKESILPKRNSDKLIARKKDWDPK |
| | KYGGFDSPTVAYSVLVVAKVEKGKSKKLKSVKELLGITIMERSSFEKNPIDFLEAKGYKEVRKDLI |
| | VKLPKYSLFELENGRKRMLASAGELQKGNELALPSKYVNFLYLASHYEKLKGSPEDNEQKQLFVE |
| | QHKHYLDEIIEQISEFSKRVILADANLDKVLSAYNKHRDKPIREQAENIIHLFTLTNLGAPAAFKYFD |
| | TTIDRKRYTSTKEVLDATFIHQSITGLYETRIDLSQLGGD |
| 66 | MTKPYSIGLDIGTNSVGWAVITDSYKVPSKKMKVLGNTSKKYVKKNLLGALLFESGITAEGRRLK |
| | RTARRRYTRRRNRILYLQEIFSTEMAKIDESFFQRLDDSFLVPDDKRDSKYPIFGNLVKEKTYHDEF |
| | PTIYHLRKYLADSSKKADLRLVYLALAHMIKYRGHFLIEGDFNSRNNDIKKNFQEFLDSYNAIFESD |
| | LSLENSKQLEEIVKDKISKSAKRERVLKLFPDEKSTGLFSEFLKLIVGNQADFKKHFDLEEKAPLQFS |
| | KDTYDEDLENLLGQIGDDFADLFLVAKKLYDAILLSGILTVTDPSTKAPLSASMIERYENHQKDLA |
| | ALKQFVRANLPEKYDEVFSDQSKDGYAGYIDGKTTQEAFYKYIKNLLSKFEGADYFLDKIEREDFL |
| | RKQRTFDNGSIPHQIHLQEMNAIIRRQGEYYPFLKENKEKIEKILTFRIPYYVGPLARGNRDFAWLTR |
| | NSDQAIRPWNFEEVVDQASSAEDFINKMTNYDLYLPEEKVLPKHSLLYETFAVYNELTKVKFIAEG |
| | LRDYQFLDSGQKKQIVNQLFKEKRKVTEKDIIHYLHNVDGYDGIELKGIEKQFNASLSTYHDLLKII |
| | KDKDFMDDPKNEEILENIVHTLTIFEDREMIKQRLAQYDSLFDEKVIKALTRRHYTGWGKLSAKLI |
| | NGIRDKKSGKTILDYLIDDDKINRNFMQLIHDDGLSFKEIIQKAQVVGKTDDLKQVVQELPGSPAIK |
| | KGILQSIKIVDELVKVMGHAPESIVIEMARENQTTNQGRRNSQQRYKLIEDGVKNLASELDGNILKE |
| | YPTDNQALQNDRLFLYYLQNGKDMYTGEPLDINHLSDYDIDHIVPQAFIKDDSIDNRVLTSSKDNR |
| | GKSDNVPSLEIVEKMKGFWQQLLDSKLISERKENNLTKAERGGLDERDKVGFIKRQLVETRQITKH |
| | VAQILDARFNTEVNEKNQKIRSVKIVTLKSNLVSNFRKEFGLYKVREINDYHHAHDAYLNAVVAK |
| | AILKKYPKLEPEFVYGDYQKYDIKRYISRSKDPKDIEKATEKYFFYSNLLNFFKEEVHYADGTIIKRE |
| | NIEYSKDTGEIAWNKEKDFATVRKVLSCPQVNIVKKTEVQTGGFSKESILPKRNSDKLIARKKDWD |
| | PKKYGGFDSPTVAYSVLVVAKVEKGKSKKLKSVKELLGITIMERSSFEKNPIDFLEAKGYKEVRKD |
| | LIVKLPKYSLFELENGRKRMLASAGELQKGNELALPSKYVNFLYLASHYEKLKGSPEDNEQKQLFV |
| | EQHKHYLDEIIEQISEFSKRVILADANLDKVLSAYNKHRDKPIREQAENIIHLFTLTNLGAPAAFKYF |
| | DTTIDRKRYTSTKEVLDATFIHQSITGLYETRIDLSQLGGD |
| 67 | MTKPYSIGLDIGTNSVGWAVITDSYKVPSKKMKVLGNTSKKYVKKNLLGALLFESGITAEGRRLK |
| | RTARRRYTRRRNRILYLQEIFSTEMAKIDESFFQRLDDSFLVPDDKRDSKYPIFGNLVEEKTYHDEFP |
| | TIYHLRKYLADSSKKADLRLVYLALAHMIKYRGHFLIEGEFNSKNNDIKKNFQEFLDSYNAIFESDL |
| | SLENNAQVEEIVKDKISKSAKRERVLKLFPDEKSTGLFSEFLKLIVGNQADFKKHFDLEEKAPLQFS |
| | KDTYDEDLENLLGQIGDDFADLFLVAKKLYDAILLSGILTVTDPSTKAPLSASMIERYENHQKDLA |
| | ALKQFIRTNLPEKYDEVFSDDSKDGYAGYIDGKTTQEEFYKYIKNLLSKLEGADYFLEKIEREDFLR |
| | KQRTFDNGSIPHQIHLQEMNAILRRQGEYYPFLQENKEKIEKILTFRIPYYVGPLARGNRDFAWLTR |
| | NSDQAIRPWNFEEIVDKASSAEDFINKMTNYDLYLPEEKVLPKHSLLYETFAVYNELTKVKFIAEGL |
| | RDYQFLDSGQKKQIVNQLFKEKRKVTEKDIIQFLHNVDGYDGIELKGIEKQFNASLSTYHDLLKIIK |
| | DKEFMDDPKNEEILENIVHTLTIFEDREMIKQRLAQYDSLFDEKVIKALTRRHYTGWGKLSAKLING |
| | ICDKQTGNTILDYLIDDDKINRNFMQLIHDDGLSFKEIIQKAQVVGKTDDVKQVVQELPGSPAIKKG |
| | ILQSIKIVDELVKVMGHEPEQIVVEMARENQTTNQGRRNSRQRYKLLDDGVKNLASDLNGNILKE |
| | YPTDNQALQNDRLFLYYLQNGKDMYTGEPLDINQLSSYDIDHIIPQAFIKDDSLDNRVLTSSKDNR |
| | GKSDNVPSLEIVEKMKGFWQQLLDSKLISERKYNNLTKAERERGGLNELDKVGFIKRQLVETRQIT |
| | KHVAQILDARFNTEVNDKKQKIRKVKIITLKSNLVSNFRKEFGLYKVREINDYHHAHDAYLNAVV |
| | AKAILKKYPKLEPEFVYGDYQKYDIKRYISRSKDPKDIEKATEKYFFYSNLLNFFKEEVHYADGTII |
| | KRENIEYSKDTGEIAWNKEKDFATVRKVLSCPQVNIVKKTEVQTGGFSKESILPKRNSDKLIARKK |
| | DWDPKKYGGFDSPTVAYSVLVVAKVEKGKSKKLKSVKELLGITIMERSSFEKNPIDFLEAKGYKEV |
| | RKDLIVKLPKYSLFELENGRKRMLASAGELQKGNELALPSKYVNFLYLASHYEKLKGSPEDNEQK |
| | QLFVEQHKHYLDEIIEQISEFSKRVILADANLDKVLSAYNKHRDKPIREQAENIIHLFTLTNLGAPAA |
| | FKYFDTTIDRKRYTSTKEVLDATFIHQSITGLYETRIDLSQLGGD |
| 68 | MTKPYSIGLDIGTNSVGWAVITDSYKVPSKKMKVLGNTSKKYVKKNLLGALLFESGITAEGRRLK |
| | RTARRRYTRRRNRILYLQEIFSTEMAKIDESFFQRLDDSFLVPDDKRDSKYPIFGNLVEEKTYHDEFP |
| | TIYHLRKYLADSSKKADLRLVYLALAHMIKYRGHFLIEGDFNSRNNDIKKNFQDFLDSYNAIFESDL |
| | SLENSKQLEEIVKDKISKSAKRERVLKLFPDEKSTGLFSEFLKLIVGNQADFKKHFDLEEKAPLQFSK |
| | DTYDEDLENLLGQIGDDFADLFLVAKKLYDAILLSGILTVTDPSTKAPLSASMIERYENHQKDLAAL |
| | KQFIQNNLQEKYDEVFSDQSKDGYAGYIDGKTTQEAFYKYIKNLLSKFEGADYFLEKIEREDFLRK |
| | QRTFDNGSIPHQIHLQEMNAILRRQGEYYPFLKENKEKIEKILTFRIPYYVGPLARGNRDFAWLTRN |
| | SDQAIRPWNFEEIVDQASSAEDFINKMTNYDLYLPEEKVLPKHSLLYETFAVYNELTKVKFIAEGLR |
| | DYQFLDSGQKKQIVTQLFKEKRKVTEKDIIQFLHNVDGYDGIELKGIEKQFNASLSTYHDLLKIIKD |
| | KEFMDDPKNEEILENIVHTLTIFEDREMIKQRLAQYDSLFDEKVIKALTRRHYTGWGKLSAKLINGI |
| | CDKQTGDTILDYLIDDDKINRNFMQLINDDGLSFKEIIQKAQVVGKTDDVKQVVQELPGSPAIKKGI |
| | LQSIKIVDELVKVMGHEPESIVIEMARENQTTNQGKRNSQQRYKRLEDGVKNLASDLNGNILKEYP |
| | TDNQALQNDRLFLYYLQNGKDMYTGEPLDINQLSSYDIDHIIPQAFIKDDSLDNRVLTSSKDNRGK |
| | SDNVPSIEVVQKRKGFWQQLLDSKLISERKENNLTKAERGGLDERDKVGFIKRQLVETRQITKHVA |
| | QILDSRFNTEVDESGKRIRKVKIITLKSNLVSNFRKEFGLYKVREINDYHHAHDAYLNAVVAKAILK |
| | KYPKLEPEFVYGDYQKYDVRKYLSTKDDKDFGKATAKLFFYSNLMNFFKKEVRFADGTIIVRPVIE |
| | YSKDTGEIAWNKEKDFATVRKVLSCPQVNIVKKTEVQTGGFSKESILPKRNSDKLIARKKDWDPK |
| | KYGGFDSPTVAYSVLVVAKVEKGKSKKLKSVKELLGITIMERSSFEKNPIDFLEAKGYKEVRKDLI |
| | VKLPKYSLFELENGRKRMLASAGELQKGNELALPSKYVNFLYLASHYEKLKGSPEDNEQKQLFVE |
| | QHKHYLDEIIEQISEFSKRVILADANLDKVLSAYNKHRDKPIREQAENIIHLFTLTNLGAPAAFKYFD |
| | TTIDRKRYTSTKEVLDATFIHQSITGLYETRIDLSQLGGD |
| 69 | MNKPYSIGLDIGTNSVGWSVITDDYKVPSKKMKVLGNTDKKYIKKNLLGALLFEGGETAEGRRLK |
| | RTARRRYTRRRNRILYLQEIFSTEMSKIDESFFQRLDDSFLVPDDKRDSKYPIFANLAQEKTYHDKFP |
| | TIYHLRKYLADSSKKADLRLVYLALAHMIKYRGHFLIEGDFNSRNNDIKKNFQNFLDSYNAIFESDL |
| | SLENSAQVEEIVKDKISKSAKRERVLKLFPDEKSTGLFSEFLKLIVGNQADFKKHFDLEEKAPLQFS |
| | KDTYDEDLENLLGQIGDDFADLFLVAKKLYDAILLSGILTVTDPSTKAPLSASMIERYENHQKDLA |
| | ALKQFIKTNLPEKYDEVFSDQSKDGYAGYIDGKTTQEAFYKYIKNTLNNVKGADYFLDKIEREDFL |
| | RKQRTFDNGSIPHQIHLQEMNAIIRRQGEHYPFLKENREKIEKILTFRIPYYVGPLARGNRDFAWLTR |
| | NSDQAIRPWNFEEVVDKASSAEEFINKMTNYDLYLPEEKVLPKHSLLYETFAVYNELTKVKFIAEG |
| | LRDYQFLDSGQKKQIVNQLFKEKRKVTEKDIIHYLHNVDGYDGIELKGIEKQFNASLSTYHDLLKII |
| | KDKEFMDDPKNEEILENIVHTLTIFEDREMIKQRLAQYDTLFDEKVIKALTRRHYTGWGKLSAKLI |
| | NGICDKQTGETILDFLIDDDKINRNFMQLINDDGLSFKEIIQKAQVVGKTDDVKQVVQELPGSPAIK |
| | KGILQSIKIVDELVKVMGHDPESIVIEMARENQTTNQGRRNSQQRYKKIEEAIKNLDSNLNSKILKE |
| | YPTDNQALQNDRLFLYYLQNGKDMYTDEELDINQLSNYDIDHIVPQAFIKDDSIDNRVLTSSKDNR |
| | GKSDNVPSLEVVQKRKAFWQQLLDSKLISERKENNLTKAERGGLDERDKVGFIKRQLVETRQITKH |
| | VAQILDSRENTEVDENGKRIRKVKIITLKSNLVSNFRKEFGLYKVREINDYHHAHDAYLNAVVAKA |
| | ILKKYPKLEPEFVYGDYQKYDIKRYISRSKDPKDIEKATEKYFFYSNLLNFFKEEVHYADGTIIKREN |
| | IEYSKDTGEVAWNKEKDFATVRKVLSCPQVNIVKKTEVQTGGFSKESILPKRNSDKLIARKKDWDP |
| | KKYGGFDSPTVAYSVLVVAKVEKGKSKKLKSVKELLGITIMERSSFEKNPIDFLEAKGYKEVRKDL |
| | IVKLPKYSLFELENGRKRMLASAGELQKGNELALPSKYVNFLYLASHYEKLKGSPEDNEQKQLFVE |
| | QHKHYLDEIIEQISEFSKRVILADANLDKVLSAYNKHRDKPIREQAENIIHLFTLTNLGAPAAFKYFD |
| | TTIDRKRYTSTKEVLDATFIHQSITGLYETRIDLSQLGGD |
| 70 | MTKPYSIGLDIGTNSVGWAVITDDYKVPSKKMKVLGNTSKKYVKKNLLGALLFESGITAEGRRLK |
| | RTARRRYTRRRNRILYLQEIFATEMAKVDESFFQRLDDSFLVPDDKRDSKYPIFGNLVEEKTYHDEF |
| | PTIYHLRKYLADSSKKADLRLVYLALAHMIKYRGHFLIEGDFNSRNNDIKKNFQNFLDSYNAIFESD |
| | LSLENSAQVEEIVKDKISKSAKRERVLKLFPDEKSTGLFSEFLKLIVGNQAEFKKHFDLEEKAPLQFS |
| | KDTYDEDLENLLGQIGDDFADLFLVAKKLYDAILLSGILTVTDPSTKAPLSASMIERYENHQKDLA |
| | ALKQFVKANLPEKYDEVFSDQSKDGYAGYIDGKTTQEAFYKYIKNLLSKFEGADYFLEKIEREDFL |
| | RKQRTFDNGSIPHQIHLQEMNAILRRQGEYYPFLKENREKIEKILTFRIPYYVGPLARGNRDFAWLT |
| | RNSDQAIRPWNFEEIVDQASSAEDFINKMTNYDLYLPEEKVLPKHSLLYETFAVYNELTKVKFIAEG |
| | LRDYQFLDSGQKKQIVNQLFKEKRKVTEKDIIHYLHNVDGYDGIELKGIEKQFNASLSTYHDLLKII |
| | KDKEFMDDPKNEEILENIVHTLTIFEDREMIKQRLAQYDTLFDEKVIKALTRRHYTGWGKLSAKLI |
| | NGISDKQTGDTILDFLIDDDKINRNFMQLINDDGLSFKEIIQKAQVVGKTDDLKQVVQELPGSPAIK |
| | KGILQSIKIVDELVKVMGHAPESIVIEMARENQTTNQGRRNSRQRYKLIEDGVKNLASDLNGNILKE |
| | YPTDNQALQNDRLFLYYLQNGKDMYTGEALDINQLSNYDIDHIVPQAFIKDDSLDNRVLTSSKDN |
| | RGKSDNVPSLEVVEKMKGFWQQLLDSKLISERKENNLTKAERGGLDERDKVGFIKRQLVETRQIT |
| | KHVAQILDARFNTEVNEKGQKIRKVKIITLKSNLVSNFRKEFGLYKVREINDYHHAHDAYLNAVV |
| | AKAILKKYPKLEPEFVYGDYQKYDIKRYISRSKDPKEIEKATEKYFFYSNLLNFFKEKVYYADGTII |
| | KRDSVEYSNDTGEIAWNKEKDFATVRKVLSCPQVNIVKKTEVQTGGFSKESILPKRNSDKLIARKK |
| | DWDPKKYGGFDSPTVAYSVLVVAKVEKGKSKKLKSVKELLGITIMERSSFEKNPIDFLEAKGYKEV |
| | RKDLIVKLPKYSLFELENGRKRMLASAGELQKGNELALPSKYVNFLYLASHYEKLKGSPEDNEQK |
| | QLFVEQHKHYLDEIIEQISEFSKRVILADANLDKVLSAYNKHRDKPIREQAENIIHLFTLTNLGAPAA |
| | FKYFDTTIDRKRYTSTKEVLDATFIHQSITGLYETRIDLSQLGGD |
| 71 | MKKPYSIGLDIGTNSVGWAVVTDDYKVPAKKMKVLGNTDKSHIKKNLLGALLFDSGNTAEDRRL |
| | KRTARRRYTRRRNRILYLQEIFAEEMNKVDESFFHRLDDSFLVPEDKRGSKYPIFATLEEEKEYHKK |
| | FPTIYHLRKHLADSKEKTDLRLIYLALAHMIKYRGHFLYEESFDIKNNDIQKIFNEFISIYDNTFEGSS |
| | LSGQNAQVEEIFTDKISKSAKRERVLKLFPDEKSTGLFSEFLKLIVGNQADFKKHFDLEEKAPLQFS |
| | KDTYDEDLENLLGQIGDDFADLFLVAKKLYDAILLSGILTVTDPSTKAPLSASMIERYENHQKDLA |
| | ALKQFIKNNLSEKYDEVFSDQSKDGYAGYIEGKTTQEAFYKYIKKAIEKIEGSDYFLDKIERENFLR |
| | KQRTFDNGSIPHQIHLQEMHAIIHRQGEYYPFLKENKEKIEKILTFRIPYYVGPLARGKSDFAWLTRK |
| | SDDAIRPWNFEEMVDKESSAEAFIHRMTNYDLYLPEEKVLPKHSLLYEKFAVYNELTKVRYVTEQ |
| | GKSFFFDANMKQEIFDGIFKKYRKVTKEKLMDFLGKEFDEFRIVDLMGLDKDNKSFNASLGTYHD |
| | LKKIVSKDFLDNPENEDILENVVLTLTLFEDREMIRKRLEKYKDVLTEEQRKKLERRHYTGWGRLS |
| | AKLINGIRDKVTRKTILDYLIDDGTSNRNFMQLIHDDTLSFVDEIRLAQGSGEAEDYREEVQNLAGS |
| | PAIKKGILQSLKIVDELVGVMGYDPEHIVVEMARENQFTNQGRRNSQQRYKKIKDAIKNLDSNLNS |
| | KILKEYPTDNQALQNDRLFLYYLQNGKDMYTGEALDINQLSNYDIDHIIPQAFIKDDSLDNRVLTSS |
| | KDNRGKSDNVPSIEVVEKMKAFWQQLLDSKLISERKYNNLTKAERERGGLNELDKVGFIKRQLVE |
| | TRQITKHVAQILDARFNTEVDEKGKRIRKVKIITLKSNLVSNFRKEFGLYKVREINDYHHAHDAYL |
| | NAVVAKAILKKYPKLEPEFVYGDYQKYDIKRYISRSKDPKDIEKATEKYFFYSNLLNFFKEEVHYA |
| | DGTIVKRENIEYSKDTGEIAWNKEKDFATVRKVLSCPQVNIVKKTEVQTGGFSKESILPKRNSDKLI |
| | ARKKDWDPKKYGGFDSPTVAYSVLVVAKVEKGKSKKLKSVKELLGITIMERSSFEKNPIDFLEAK |
| | GYKEVRKDLIVKLPKYSLFELENGRKRMLASAGELQKGNELALPSKYVNFLYLASHYEKLKGSPE |
| | DNEQKQLFVEQHKHYLDEIIEQISEFSKRVILADANLDKVLSAYNKHRDKPIREQAENIIHLFTLTNL |
| | GAPAAFKYFDTTIDRKRYTSTKEVLDATFIHQSITGLYETRIDLSQLGGD |
| 72 | MTKPYSIGLDIGTNSVGWAVITDSYKVPSKKMKVLGNTSKKYVKKNLLGALLFESGITAEGRRLK |
| | RTARRRYTRRRNRILYLQEIFSTEMAKIDESFFQRLDDSFLVPDDKRDSKYPIFGNLVEEKTYHDEFP |
| | TIYHLRKYLADSSKKADLRLVYLALAHMIKYRGHFLIEGDFNSRNNDIKKNFQDFLDSYNAIFESDL |
| | SLENSKQLEEIVKDKISKSAKKDRLLKLFPGEKNSGIFSEFLKLIVGNQADFRKYFNLDEKASLHFSK |
| | ESYDEDLETLLGHIGDDYSDVFLKAKKLYDAILLSGILTVTDNETEAPLSSAMIMRYKEHEEDLALL |
| | KAYIRKISLETYNEVFKDDTKNGYAGYIDGKTNQEDFYVYLKKLLTGLEGADYFLEKINREDLLRK |
| | QRTFDNGSIPYQIHLQEMRAIIDKQAKFYPFLAKNKERIEKILTFRIPYYVGPLARGNSDFAWSIRKR |
| | NEKITPWNFEDVIDKESSAEAFINRMTSFDLYLPEEKVLPKHSLLYETFTVYNELTKVRFIAESMRD |
| | YQFLDSKQKKDIVRLYFKGKRKVTDKDIIDYLHAIDGYDGIELKGIEKQFNSSLSTYHDLLKIIKDKE |
| | FLDDASNEAIIEEIIHTLTIFEDREMIKQRLSKFENIFDKSVLKKLSRRHYTGWGKLSAKLINGIRDEK |
| | SGKTILDYLIDDGVSNRNFMQLIHDDTLSFKKKIQKAQIIGDQDNIKQVVQSLPGSPAIKKGILQSIKI |
| | VDELVKVMGRNPESIVVEMARENQYTNQGKSNSQQRLKRLEESLKELGSKILKENVPAKLSKIDN |
| | NALQNERLYLYYLQNGKDMYTGDDLDIDRLSNYDIDHIIPQAFLKDNSIDNKVLVSSASNRGKSDD |
| | VPSLDVVKKRKTFWHQLLKSQLISQRKFDNLTKAERGGLTEKDKAGFIKRQLVETRQITKHVAQIL |
| | DSRFNTKRDDNDKVIRNVKIITLKSSLVSQFRKEFGLYKVREINDYHHAHDAYLNAVVATAILKKY |
| | PKLEPEFVYGDYKTYTYDELKQGDLRQIKRADIDKSLGKATAKMFFYSNLMNFFKKEVRLADGTV |
| | IVRPVIEYSKDTGEIAWNKEKDFATVRKVLSCPQVNIVKKTEVQTGGFSKESILPKRNSDKLIARKK |
| | DWDPKKYGGFDSPTVAYSVLVVAKVEKGKSKKLKSVKELLGITIMERSSFEKNPIDFLEAKGYKEV |
| | RKDLIVKLPKYSLFELENGRKRMLASAGELQKGNELALPSKYVNFLYLASHYEKLKGSPEDNEQK |
| | QLFVEQHKHYLDEIIEQISEFSKRVILADANLDKVLSAYNKHRDKPIREQAENIIHLFTLTNLGAPAA |
| | FKYFDTTIDRKRYTSTKEVLDATFIHQSITGLYETRIDLSQLGGD |
| 73 | MTKPYSIGLDIGTNSVGWAVITDSYKVPSKKMKVLGNTSKKYVKKNLLGALLFESGITAEGRRLK |
| | RTARRRYTRRRNRILYLQEIFSTEMAKIDESFFQRLDDSFLVPDDKRDSKYPIFGNLVEEKVYHDEF |
| | PTIYHLRKYLADSSKKADLRLVYLALAHMIKYRGHFLIEGEFNSKNNDIKKNFQEFLDSYNAIFESD |
| | LSLENSKQLEEIVKDKISKSAKRERVLKLFPDEKSTGLFSEFLKLIVGNQADFKKHFDLEEKAPLQFS |
| | KDTYDEDLENLLGQIGDDFADLFLVAKKLYDAILLSGILTVTDPSTKAPLSASMIERYENHQKDLA |
| | ALKQFIKTNLPEKYDEVFSDQSKDGYAGYIDGKTTQEAFYKYIKNLLSKFEGADYFLDKIEREDFLR |
| | KQRTFDNGSIPHQIHLQEMNAIIRRQGEHYPFLKENKEKIEKILTFRIPYYVGPLARGNRDFAWLTR |
| | NSDQAIRPWNFEEIVDKASSAEDFINKMTNYDLYLPEEKVLPKHSLLYETFAVYNELTKVKFIAEDL |
| | TRYQFLDKKQKKDIFDTFFKAEDKRKVTEKDIIHYLHNVDGYDGIELKGIEKQFNASLSTYHDLLKI |
| | IKDKEFMDDAKNEEILENIIHTLTIFEDREMIKQRLAQYDSLFDEKVIKALTRRHYTGWGKLSAKLI |
| | NGIRDKKSGKTILDYLIDDGEINRNFMQLIHDDGLSFKEIIQKAQVFGKTDDIKQVVQNLPGSPAIKK |
| | GILQSIKIVDELVKVMGKDPEHIVVEMARENQFTNQGRRNSQQRYKKIKDAIKNLDSNLNSKILKE |
| | YPTNNQALQNDRLFLYYLQNGKDMYTGEPLEINQLSNYDIDHIIPQAFIKDDSLDNRVLTSSKDNR |
| | GKSDNVPSIEVVEKMKGFWKQLLDSKLISERKYNNLTKAERERDGLNELDKVGFIKRQLVETRQIT |
| | KHVAQILDARFNTEVNEKNQKIRNVKIITLKSNLVSNFRKEFGLYKVREINDYHHAHDAYLNAVV |
| | AKAILKKYPKLEPEFVYGDYQKYDLKRYISRSKDPKEIEKATQKYFFYSNLLNFFKEEVHYADGTI |
| | VKRENIEYSKDTGEIAWNKEKDFATVRKVLSCPQVNIVKKTEVQTGGFSKESILPKRNSDKLIARK |
| | KDWDPKKYGGFDSPTVAYSVLVVAKVEKGKSKKLKSVKELLGITIMERSSFEKNPIDFLEAKGYKE |
| | VRKDLIVKLPKYSLFELENGRKRMLASAGELQKGNELALPSKYVNFLYLASHYEKLKGSPEDNEQ |
| | KQLFVEQHKHYLDEIIEQISEFSKRVILADANLDKVLSAYNKHRDKPIREQAENIIHLFTLTNLGAPA |
| | AFKYFDTTIDRKRYTSTKEVLDATFIHQSITGLYETRIDLSQLGGD |
| 74 | MTKPYSIGLDIGTNSVGWAVITDSYKVPSKKMKVLGNTSKKYVKKNLLGALLFESGITAEGRRLK |
| | RTARRRYTRRRNRILYLQEIFSTEMAKIDESFFQRLDDSFLVPDDKRGSKYPIFGNLAKEKTYHDEF |
| | PTIYHLRKYLADSSKKADLRLVYLALAHMIKYRGHFLIEGDFNSRNNDIKKNFQNFLDSYNAIFESD |
| | LSLENSEQLEEIVKDKISKSAKRERVLKLFPDEKSTGLFSEFLKLIVGNQADFKKHFDLEEKAPLQFS |
| | KDTYDEDLENLLGQIGDDFADLFLVAKKLYDAILLSGILTVTDPSTKAPLSASMIERYENHQKDLA |
| | ALKQFIQNNLQEKYDEVFSDQSKDGYAGYIDGKTTQEAFYKYIKNLLSKLEGADYFLDKIEREDFL |
| | RKQRTFDNGSIPHQIHLQEMNAIIRRQGEHYPFLKENKEKIEKILTFRIPYYVGPLARGNRDFAWLTR |
| | NSDQAIRPWNFEEIVDQASSAEDFINKMTNYDLYLPEEKVLPKHSLLYETFAVYNELTKVKFIAEGL |
| | RDYQFLDSGQKKQIVNQLFKEKRKVTEKDIIHYLHNVDGYDGIELKGIEKQFNASLSTYHDLLKIIK |
| | DKEFMDDPKNEEILENIVHTLTIFEDREMIKQRLAQYDTLFDEKVIKALTRRHYTGWGKLSAKLIN |
| | GISDKQTGKTILDFLIDDDKINRNFMQLINDDGLSFKEIIQKAQVIGETDNIKQVVQNLPGSPAIKKGI |
| | LQSIKIVDELVKVMGHDPESIVIEMARENQTTNQGRRNSQQRYKRIEEAIKNLDSNLNSKILKEYPT |
| | DNQALQNDRLFLYYLQNGKDMYTGQELDINQLSNYDIDHIVPQAFIKDDSLDNRVLTSSKDNRGK |
| | SDNVPSIEIVQKRKAFWQQLLDSKLISERKENNLTKAERGGLNERDKVGFIKRQLVETRQITKHVA |
| | QILDARFNTEVNAKGKKIRKVKIITLKSNLVSNFRKEFGLYKVREINDYHHAHDAYLNAVVAKAIL |
| | KKYPKLEPEFVYGDYQKYDVKRYISRSKDPKDIEKATEKYFFYSNLLNFFKEEVHYADGTIVKREN |
| | IEYSKDTGEIAWNKEKDFATVRKVLSCPQVNIVKKTEVQTGGFSKESILPKRNSDKLIARKKDWDP |
| | KKYGGFDSPTVAYSVLVVAKVEKGKSKKLKSVKELLGITIMERSSFEKNPIDFLEAKGYKEVRKDL |
| | IVKLPKYSLFELENGRKRMLASAGELQKGNELALPSKYVNFLYLASHYEKLKGSPEDNEQKQLFVE |
| | QHKHYLDEIIEQISEFSKRVILADANLDKVLSAYNKHRDKPIREQAENIIHLFTLTNLGAPAAFKYFD |
| | TTIDRKRYTSTKEVLDATFIHQSITGLYETRIDLSQLGGD |
| 75 | MTKPYSIGLDIGTNSVGWAVITDSYKVPSKKMKVLGNTSKKYVKKNLLGALLFESGITAEGRRLK |
| | RTARRRYTRRRNRILYLQEIFSTEMAKIDESFFQRLDDSFLVPDDKRDSKYPIFGNLVKEKTYHDEF |
| | PTIYHLRKYLADSSKKADLRLVYLALAHMIKYRGHFLIEGDFNSRNNDIKKNFQDFLDSYNAIFESD |
| | LSLENSKQLEEIVKDKISKSAKKERLLKLFPGEKNSGILAEFLKLIVGNQADFRKYFNLDEKASLHES |
| | KESYDEDLETLLGHIGDDYSDVFLKAKKLYDAILLSGILTVTDNETEAPLSSAMIMRYKEHEEDLAL |
| | LKAYIRNISLETYNEVFKDDTKNGYAGYIDGKTNQEDFYVYLKKLLTGLEGADYFLEKINREDLLR |
| | KQRTFDNGSIPYQIHLQEMRAILDKQAKFYPFLAKNKERIEKILTFRIPYYVGPLARGNSDFAWSIRK |
| | RNEKITPWNFEDVIDKESSAEAFINRMTSFDLYLPEEKVLPKHSLLYETFTVYNELTKVRFIAESMR |
| | DYQFLDSKQKKDIVRLYFKGKRKVTDKDIIDYLHAIDGYDGIELKGIEKQFNSSLSTYHDLLKIIKD |
| | KEFLDDSSNEAIIEEIIHTLTIFEDREMIKQRLSKFENIFDKSVLKKLSRRHYTGWGKLSAKLINGIRD |
| | EKSGKTILDYLIDDGISNRNFMQLIHDDTLSFKKKIQKAQIIGDQDNIKQVVQSLPGSPAIKKGILQSI |
| | KIVDELVKVMGRRPESIVVEMARENQYTNQGKSNSQQRLKRLEESLKELGSKILKENVPAKLSKID |
| | NNALQNERLYLYYLQNGKDMYTGQELDIDRLSNYDIDHIIPQAFLKDNSIDNKVLVSSASNRGKSD |
| | DVPSLDVVKKRKTFWHQLLKSKLISQRKFDNLTKAERGGLTEDDKAGFIKRQLVETRQITKHVAQI |
| | LDARFNTEVNEKNQKIRTVKIVTLKSNLVSNFRKEFELYKVREINDYHHAHDAYLNAVVAKAILTK |
| | YPKLEPEFVYGDYPKYNSFRERKSATEKVYFYSNIMNTFKKSITLADGTVIDRPLIEVNEETGESVW |
| | NKEKDFATVRKVLSCPQVNIVKKTEVQTGGFSKESILPKRNSDKLIARKKDWDPKKYGGFDSPTVA |
| | YSVLVVAKVEKGKSKKLKSVKELLGITIMERSSFEKNPIDFLEAKGYKEVRKDLIVKLPKYSLFELE |
| | NGRKRMLASAGELQKGNELALPSKYVNFLYLASHYEKLKGSPEDNEQKQLFVEQHKHYLDEIIEQI |
| | SEFSKRVILADANLDKVLSAYNKHRDKPIREQAENIIHLFTLTNLGAPAAFKYFDTTIDRKRYTSTK |
| | EVLDATFIHQSITGLYETRIDLSQLGGD |
| 76 | MNKPYSIGLDIGTNSVGWAVITDDYKVPSKKMKVLGNTSKKYVKKNLLGALLFESGITAEGRRLK |
| | RTARRRYTRRRNRILYLQEIFSTEMAKIDESFFQRLDDSFLVPDDKRDSKYPIFGNLAKEKTYHDEF |
| | PTIYHLRKYLADSTKKADLRLVYLALAHMIKYRGHFLIEGDFNSRNNDIKKNFQDFLDSYNAIFESD |
| | LSLENSKQLEEIVKDKISKSAKRERVLKLFPDEKSTGLFSEFLKLIVGNQADFKKHFDLEEKAPLQFS |
| | KDTYDEDLENLLGQIGDDFADLFLVAKKLYDAILLSGILTVTDPSTKAPLSASMIDRYENHQKDLA |
| | ALKQFIRKNLPEKYDEVFSDESKDGYAGYIDGKTTQENFYKYIKNAISKIEGADYFLEKIEREDFLR |
| | KQRTFDNGSIPHQIHLQEMNAILHNQGEYYPFLKENKEKIEKILTFRIPYYVGPLARGNRDFAWLTR |
| | NSDQAIRPWNFEEVVDQASSAEEFINKMTNYDLYLPEEKVLPKHSLLYETFAVYNELTKVKFIAEG |
| | LRDYQFLDSGQKKQIVNQLFKEKRKVTEKDIIHYLHNVDGYDGIELKGIEKQFNASLSTYHDLLKII |
| | KDKEFMDDPKNEEILENIVHTLTIFEDREMIKQRLAQYDTLFDEKVIKALTRRHYTGWGKLSAKLI |
| | NGIRDKKSGKTILDYLIDDDKINRNFMQLIHDDGLSFKEIIQKAQVVGDSDNLKQVVQNLPGSPAIK |
| | KGILQSIKIVDELVKVMGHDPEQIVIEMARENQTTNEGRRNSRQRYKLIEDGVKNLAKDLDGNILK |
| | EYPTDNQALQNDRLFLYYLQNGKDMYTGEPLDINQLSSYDIDHIVPQAFIKDDSLDNRVLTSSKDN |
| | RGKSDNVPSLEVVQKRKAFWQQLLDSKLISEHKFNNLTKAERGGLDERDKVGFIRRQLVETRQITK |
| | HVAQILDARFNTEVNEKNQKIRTVKIVTLKSNLVSNFRKEFGLYKVREINDYHHAHDAYLNAVVA |
| | KAILKKYPKLEPEFVYGDYQKYDLKRYISKSKDPKEVEKATQKYFFYSNLLNFFKEEVHYADGTII |
| | KRENIEYSKDTGEIAWNKEKDFATVRKVLSCPQVNIVKKTEVQTGGFSKESILPKRNSDKLIARKK |
| | DWDPKKYGGFDSPTVAYSVLVVAKVEKGKSKKLKSVKELLGITIMERSSFEKNPIDFLEAKGYKEV |
| | RKDLIVKLPKYSLFELENGRKRMLASAGELQKGNELALPSKYVNFLYLASHYEKLKGSPEDNEQK |
| | QLFVEQHKHYLDEIIEQISEFSKRVILADANLDKVLSAYNKHRDKPIREQAENIIHLFTLTNLGAPAA |
| | FKYFDTTIDRKRYTSTKEVLDATFIHQSITGLYETRIDLSQLGGD |
| 77 | MTKPYSIGLDIGTNSVGWAVITDSYKVPSKKMKVLGNTSKKYVKKNLLGALLFESGITAEGRRLK |
| | RTARRRYTRRRNRILYLQEIFSTEMNKIDESFFQRLDDSFLVPDDKRDSKYPIFGNLVKEKTYHDEF |
| | PTIYHLRKYLADSSKKADLRLVYLALAHMIKYRGHFLIEGDFNSRNNDIKKNFQNFLDSYNAIFESD |
| | LSLENSKQLEEIVKDKISKSAKRERVLKLFPDEKSTGLFSEFLKLIVGNQADFKKHFDLEEKAPLQFS |
| | KDTYDEDLENLLGQIGDGFADLFLVAKKLYDAILLSGILTVTDPSTKAPLSASMIERYENHQKDLAT |
| | LKQFIKTNLPEKYDEVFSDQSKDGYAGYIDGKTTQEEFYKYLKNTLSKIEGSDYFLDKIEREDFLRK |
| | QRTFDNGSIPHQIHLQEMNAIIRRQGEHYPFLKENKEKIEKILTFRIPYYVGPLARGNRDFAWLTRNS |
| | DQAIRPWNFEEIVDQASSAEDFINKMTNYDLYLPEEKVLPKHSLLYETFAVYNELTKVKFIAEGLR |
| | DYQFLDSGQKKQIVNQLFKDKRKVTEKDIIQYLHNVDGYDGIELKGIEKQFNASLSTYHDLLKIIKD |
| | KEFMDDPKNEEILENIVHTLTIFEDREMIKQRLAKYANIFDKSVLKKLARRHYTGWGKLSAKLINGI |
| | YDKQSRKTILDYLIDDDKINRNFMQLINDDGLSFKEIIQKAQVVGKTDDVKQVVQELPGSPAIKKGI |
| | LQSIKIVDELVKVMGHAPESIVIEMARENQTTARGKKNSQQRYKRIEDSLKNLASGLDSNILKENPT |
| | DNIQLQNDRLFLYYLQNGKDMYTGESLDINQLSSYDIDHIIPQAFIKDDSLDNRVLTSSKDNRGKSD |
| | NVPSIEVVQKRKAFWQQLLDSKLISERKENNLTKAERGGLDERDKVGFIRRQLVETRQITKHVAQI |
| | LDARFNTEVNEKKQKIRKVKIITLKSNLVSNFRKEFRLYKVREINDYHHAHDAYLNAVVAKAILKK |
| | YPKLEPEFVYGDYQKYDLKRYISKSKDPKEIEKATQKYFFYSNLLNFFKEEVHYADGTIIKRENIEY |
| | SKDTGEIAWNKEKDFATVRKVLSCPQVNIVKKTEVQTGGFSKESILPKRNSDKLIARKKDWDPKK |
| | YGGFDSPTVAYSVLVVAKVEKGKSKKLKSVKELLGITIMERSSFEKNPIDFLEAKGYKEVRKDLIV |
| | KLPKYSLFELENGRKRMLASAGELQKGNELALPSKYVNFLYLASHYEKLKGSPEDNEQKQLFVEQ |
| | HKHYLDEIIEQISEFSKRVILADANLDKVLSAYNKHRDKPIREQAENIIHLFTLTNLGAPAAFKYFDT |
| | TIDRKRYTSTKEVLDATFIHQSITGLYETRIDLSQLGGD |
| 78 | MTKPYSIGLDIGTNSVGWAVITDNYKVPSKKMKVLGNTSKKYVKKNLLGALLFESGITAEGRRLK |
| | RTARRRYTRRRNRILYLQEIFSTEMAKIDESFFQRLDDSFLVPDDKRDSKYPIFGNLVKEKTYHDEF |
| | PTIYHLRKYLADSQKKADLRLVYLALAHMIKYRGHFLIEGDFNSRNNDIKKNFQNFLDSYNAIFES |
| | DLSLENSKQLEEIVKDKISKSAKRERVLKLFPDEKSTGLFSEFLKLIVGNQADFKKHFDLEEKAPLQF |
| | SKDTYDEDLENLLGQIGDDFADLFLVAKKLYDAILLSGILTVTDPSTKAPLSASMIERYENHQKDLA |
| | ALKQFIQNNLQEKYDEVFSDQSKDGYAGYIDGKTTQEAFYKYIKNLLSKLEGSDYFLDKIEREDFL |
| | RKQRTFDNGSIPHQIHLQEMNAIIRRQGEHYPFLKENKEKIEKILTFRIPYYVGPLARGNRDFAWLTR |
| | NSDQAIRPWNFEEIVDKASSAEDFINKMTNYDLYLPEEKVLPKHSLLYETFAVYNELTKVKFIAEGL |
| | RDYQFLDSGQKKQIVNQLFKEKRKVTEKDIIQFLHTVDGYDGIELKGIEKQFNASLSTYHDLLKIIK |
| | DKEFMDDPKNEEILENIVHTLTIFEDREMIKQRLAQYDSLFDEKVIKALIRRHYTGWGKLSAKLING |
| | IYDKQSKKTILDYLIDDDKINRNFMQLINDDGLSFKDIIQKAQVVGKTNDVKQVVQELPGSPAIKKG |
| | ILQSIKLVDELVKVMGHAPESIVIEMARENQTTARGKKNSQQRYKRIEDALKNLAPGLDSNILKEHP |
| | TDNIQLQNDRLFLYYLQNGRDMYTGEPLDINQLSSYDIDHIIPQAFIKDDSLDNRVLTSSKDNRGKS |
| | DNVPSLEVVQKRKAFWQQLLDSKLISERKENNLTKAERGGLDERDKVGFIKRQLVETRQITKHVA |
| | QILDARFNTEVDENNQRIRKVKIITLKSNLVSNFRKEFGLYKVREINDYHHAHDAYLNAVVAKAIL |
| | KKYPKLEPEFVYGDYQKYDIKRYIRKFQPSKEIDKATEKYFFYSNLLNFFKEEVHYADGTIIKRENIE |
| | YSKDTGEIAWNKEKDFATVRKVLSCPQVNIVKKTEVQTGGFSKESILPKRNSDKLIARKKDWDPK |
| | KYGGFDSPTVAYSVLVVAKVEKGKSKKLKSVKELLGITIMERSSFEKNPIDFLEAKGYKEVRKDLI |
| | VKLPKYSLFELENGRKRMLASAGELQKGNELALPSKYVNFLYLASHYEKLKGSPEDNEQKQLFVE |
| | QHKHYLDEIIEQISEFSKRVILADANLDKVLSAYNKHRDKPIREQAENIIHLFTLTNLGAPAAFKYFD |
| | TTIDRKRYTSTKEVLDATFIHQSITGLYETRIDLSQLGGD |
| 79 | MKKPYSIGLDIGTNSVGWAVVTDDYKVPAKKMKVLGNTDKSHIKKNLLGALLFDSGETAEGRRL |
| | KRTARRRYTRRRNRILYLQEIFAEEMNKVDESFFHRLDDSFLVPEDKRGSKYPIFATLEEEKEYHKK |
| | FPTIYHLRKHLADSKEKTDLRLIYLALAHMIKYRGHFLYEESFDIKNNDIQKIFNEFISIYDNTFEGSS |
| | LSGQNAQVEAIFTDKISKSAKRERVLKLFPDEKSTGLFSEFLKLIVGNQADFKKHFDLEEKAPLQFS |
| | KDTYDEDLENLLGQIGDDFADLFLVAKKLYDAILLSGILTVKDPSTKAPLSASMIKRYAEHHEDLE |
| | KLKEFVKANAPELYHDIFKDESKNGYAGYIENGVKQDEFYKYLKNTLSKIEGSDYFLDKIEREDFL |
| | RKQRTFDNGSIPHQIHLQEMNAIIRRQGEYYPFLQENKEKIEKILTFRIPYYVGPLARGNRDFAWLTR |
| | NSDEAIRPWNFEEVVDKASSAEDFINKMTNYDLYLPEEKVLPKHSLLYEKFAVYNELTKVRYVTE |
| | QGKSFFFDANMKQEIFDKTFKVYRKVTKEKLMDFLGKEFDEFRIVDLIGLDKDNKSFNASLGTYHD |
| | LKKIVSKDFLDNPENEDILENVVLTLTLFEDREMIRKRLEKYKDVLTEEQQKKLERRHYTGWGRLS |
| | AKLINGIRNKTTRKTILDYLIDDGTSNRNFMQLIHDDTLSFVDEIRLAQGSGEAEDYRAEVQNLAGS |
| | PAIKKGILQSLKIVDELIEVMGYEPEHIVVEMARENQFTNQGRRNSQQRYKKIENAIKNLDSNLNSK |
| | ILKEYPTNNQALQNDRLFLYYLQNGKDMYTGEALDINQLSNYDIDHIIPQAFIKDDSLDNRVLTSSK |
| | DNRGKSDNVPSIEVVEKMKAFWQQLLDSKLISERKYNNLTKAERERGGLNELDKVGFIKRQLVET |
| | RQITKHVAQILDARFNTEVDENNQRIRKVKIITLKSNLVSNFRKEFGLYKVREINDYHHAHDAYLN |
| | AVVAKAILKKYPKLEPEFVYGDYQKYDIKRYISRSKDPKDIEKATEKYFFYSNLLNFFKEEVHYAD |
| | GTIIKRENIEYSKDTGEIAWNKEKDFATVRKVLSCPQVNIVKKTEVQTGGFSKESILPKRNSDKLIAR |
| | KKDWDPKKYGGFDSPTVAYSVLVVAKVEKGKSKKLKSVKELLGITIMERSSFEKNPIDFLEAKGY |
| | KEVRKDLIVKLPKYSLFELENGRKRMLASAGELQKGNELALPSKYVNFLYLASHYEKLKGSPEDN |
| | EQKQLFVEQHKHYLDEIIEQISEFSKRVILADANLDKVLSAYNKHRDKPIREQAENIIHLFTLTNLGA |
| | PAAFKYFDTTIDRKRYTSTKEVLDATFIHQSITGLYETRIDLSQLGGD |
| 80 | MTKPYSIGLDIGTNSVGWAVITDSYKVPSKKMKVLGNTSKKYVKKNLLGALLFESGITAEGRRLK |
| | RTARRRYTRRRNRILYLQEIFSTEMAKIDESFFQRLDDSFLVPDDKRDSKYPIFGNLVKEKTYHDEF |
| | PTIYHLRKYLADSSKKADLRLVYLALAHMIKYRGHFLIEGDFDSRNNDIKKNYQNFLDSYNAIFES |
| | DLSLENNAQVEEIVKDKISKSAKRERVLKLFPDEKSTGLFSEFLKLIVGNQADFKKHFDLEEKAPLQ |
| | FSKDTYDEDLENLLGQIGDDFADLFLVAKKLYDAILLSGILTVTDPSTKAPLSASMIERYENHQKDL |
| | AALKQFIKNNLSEKYDEVFSDQSKDGYAGYINGKTTQEAFYKYIKNLLSKLEGADYFLDKIEREDF |
| | LRKQRTFDNGSIPHQIHLQEMNAIIRRQGEHYPFLKENKEKIEKILTFRIPYYVGPLARGNRDFAWLT |
| | RNSDQAIRPWNFEEVVDQASSAEDFINKMTNYDLYLPEEKVLPKHSLLYETFAVYNELTKVKFIAE |
| | GLRDYQFLDSGQKKQIVNQLFKEKRKVTEKDIIQFLHNVDGYDGIELKGIEKQFNASLSTYHDLLKI |
| | IKDKEFMDDPKNEEILENIVHTLTIFEDREMIKQRLAQYASIFDEKVIKALTRRHYTGWGRLSAKLIN |
| | GIRDKQSRKTILDYLIDDDKINRNFMQLINDDGLSFKDIIQKAQVVGKTDDVKQVVQELPGSPAIKK |
| | GILQSIKIVDELVKVMGKDPESIVIEMARENQTTNQGRRNSQQRYKRIEEAIKNLSIDLNSKILKEYP |
| | TDNQALQNDRLFLYYLQNGKDMYTGEELDINQLSNYDIDHIVPQAFIKDDSLDNRVLTSSKDNRG |
| | KSDNVPSIEVVQRRKAFWQQLLDSKLISERKENNLTKAERGGLDERDKVGFIKRQLVETRQITKHV |
| | AQILDARFNTEVNENNQRIRKVKIITLKSNLVSNFRKEFGLYKVREINDYHHAHDAYLNAVVAKAI |
| | LKKYPKLEPEFVYGDYQKYDIKRYISRSKDPKEIEKATEKYFFYSNLLNFFKEEVHYADGTIVKREN |
| | IEYSKDTGEVAWNKEKDFATVRKVLSCPQVNIVKKTEVQTGGFSKESILPKRNSDKLIARKKDWDP |
| | KKYGGFDSPTVAYSVLVVAKVEKGKSKKLKSVKELLGITIMERSSFEKNPIDFLEAKGYKEVRKDL |
| | IVKLPKYSLFELENGRKRMLASAGELQKGNELALPSKYVNFLYLASHYEKLKGSPEDNEQKQLFVE |
| | QHKHYLDEIIEQISEFSKRVILADANLDKVLSAYNKHRDKPIREQAENIIHLFTLTNLGAPAAFKYFD |
| | TTIDRKRYTSTKEVLDATFIHQSITGLYETRIDLSQLGGD |
| 81 | MTKPYSIGLDIGTNSVGWAVITDSYKVPSKKMKVLGNTSKKYVKKNLLGALLFESGITAEGRRLK |
| | RTARRRYTRRRNRILYLQEIFSTEMAKIDESFFQRLDDSFLVPDDKRDSKYPIFGNLVEEKTYHDEFP |
| | TIYHLRKYLADSSKKADLRLVYLALAHMIKYRGHFLIEGDENSKNNDIKKNFQEFLDSYNAIFESDL |
| | SLENSKQLEEIVKDKISKSAKRERVLKLFPDEKSTGLFSEFLKLIVGNQADFKKHFDLEEKAPLQFSK |
| | DTYDEDLENLLGQIGDDFADLFLVAKKLYDAILLSGILTVTDPSTKAPLSASMIERYENHQKDLAAL |
| | KQFIQNNLQEKYDEVFSDQSKDGYAGYIDGKTTQEAFYKYIKNLLSKLEGADYFLDKIEREDFLRK |
| | QRTFDNGSIPHQIHLQEMNAILRRQGEHYPFLKENKEKIEKILTFRIPYYVGPLARGNRDFAWLTRN |
| | SDQAIRPWNFEEIVDQASSAEDFINKMTNYDLYLPEEKVLPKHSLLYETFAVYNELTKVKFIAEGLR |
| | DYQFLDSGQKKQIVNQLFKEKRKVTEKDIIQFLHNVDGYDGIELKGIEKQFNASLSTYHDLLKIIKD |
| | KDFMDDPKNEEILENIVHTLTIFEDREMIKKRLEQYDSIFDEKVIKALTRRHYTGWGRLSAKLINGIR |
| | DKQSNKTILDYLIDDDKINRNFMQLINDDGLSFKSIISKAQAGSHSDDLKEVVGELAGSPAIKKGILQ |
| | SLKIVDELVKVMGYEPEQIVVEMARENQTTNQGRRNSRQRYKLLDDGIKNLAKDLDGNILKEYPT |
| | NNQALQNDRLFLYYLQNGKDMYTGEALDINQLSSYDIDHIVPQAFIKDDSIDNRVLTSSKDNRGKS |
| | DNVPSLEVVQKRKAFWQQLLDSKLISERKENNLTKAERGGLDERDKVGFIRRQLVETRQITKHVA |
| | QILDARFNTEVNENNQKIRKVKIITLKSNLVSNFRKEFGLYKVREINDYHHAHDAYLNAVVAKAIL |
| | KKYPKLEPEFVYGDYQKYDIKRYISRSKDPKDIEKATEKYFFYSNLLNFFKEEVHYADGTIIKRENIE |
| | YSKDTGEIAWNKEKDFATVRKVLSCPQVNIVKKTEVQTGGFSKESILPKRNSDKLIARKKDWDPK |
| | KYGGFDSPTVAYSVLVVAKVEKGKSKKLKSVKELLGITIMERSSFEKNPIDFLEAKGYKEVRKDLI |
| | VKLPKYSLFELENGRKRMLASAGELQKGNELALPSKYVNFLYLASHYEKLKGSPEDNEQKQLFVE |
| | QHKHYLDEIIEQISEFSKRVILADANLDKVLSAYNKHRDKPIREQAENIIHLFTLTNLGAPAAFKYFD |
| | TTIDRKRYTSTKEVLDATFIHQSITGLYETRIDLSQLGGD |
| 82 | MKKSYSIGLDIGTNSVGWSVVTDDYKVPAKKMKVFGNTDKKYIKKNLLGALLFDSGETAEGTRL |
| | KRTARRRYTRRRNRILYLQEIFAKEMAKVDESFFQRLEESFLTDDDKNFDSHPIFGNKAEEDAYHE |
| | QFPTIYHLRKHLADSSEKADLRLVYLALAHMIKFRGHFLIEGQLKAENTNVQTLFNDFVEVYDKTV |
| | EESHLAEITVDALSILTEKVSKSRRLENLLKYYPTEKKNTLFGNLIALSLGLQPNFKTNFKLSEDAKL |
| | QFSKDTYEEDLEELLGKIGDEYADLFTSAKNLYDAILLSGILTVDDKSTKAPLSASMLKRYVEHHE |
| | DLEKLKDFVKANKPELYHDIFKDETKNGYAGYIENGVKQDEFYKYLKNTLSKIAGSDYFLDKIERE |
| | DFLRKQRTFDNGSIPHQVHLQEMRAILRRQGEYYPFLKENQAKIEKILTFRIPYYVGPLARKDSRFA |
| | WAEYHSDEKITPWNFDKVIDKESSARAFIEHMTNNDLYLPEEKVLPKHSPLYEKYTVYNELTKVK |
| | YVTEQGKSSFFDANMKQEIFEHVFKKNRKVTKDKFLNYLNKEFPEYRIHDLIGLDKENKSFNASLG |
| | TYHDLKKILDKSFLDNKENEEIIEDVVLTLTLFEDRDMIRQRLQKYSDIFTKDQLKKLERRHYTGW |
| | GRLSYKLINGIRDKQSNKTILDYLLEDDGVKKNINRNFMQLINDDSLSFKTIIQEAQVIGDIDDIKAV |
| | VHDLPGSPAIKKGILQSVKIVDELVKVMGHNPDNIVIEMARENQTTNRGRSQSQQRLKKLQESLKE |
| | LGSNILNEEKPSYIEDKVENSHLQNDRLFLYYIQNGKDMYTGDELDIDHLSDYDIDHIIPQAFIKDNS |
| | IDNRVLTSSAKNRGKSDDVPSLDIVRERKADWIRLYKAGLISKRKFDNLTKAERGGLTENDKAGFI |
| | KRQLVETRQITKHVAQILDARFNTKRDENDKVIRDVKIITLKSNLVSQFRKEFGFYKVREINDYHHA |
| | HDAYLNAVVGTALIKKYPKLESEFVYGDYKVYDVRKMIAKSEQEIGKATAKRFFYSNIMNFFKTEI |
| | KLANGEIRKRPLIETNEETGEVVWNKERDFAIVRKVLSCPQVNIVKKTEVQTGGFSKESILPKRNSD |
| | KLIARKKDWDPKKYGGFDSPTVAYSVLVVAKVEKGKSKKLKSVKELLGITIMERSSFEKNPIDFLE |
| | AKGYKEVRKDLIVKLPKYSLFELENGRKRMLASAGELQKGNELALPSKYVNFLYLASHYEKLKGS |
| | PEDNEQKQLFVEQHKHYLDEIIEQISEFSKRVILADANLDKVLSAYNKHRDKPIREQAENIIHLFTLT |
| | NLGAPAAFKYFDTTIDRKRYTSTKEVLDATFIHQSITGLYETRIDLSQLGGD |
| 83 | MTKPYSIGLDIGTNSVGWAVITDDYKVPSKKMKVLGNTSKKYVKKNLLGALLFESGITAEGRRLK |
| | RTARRRYTRRRNRILYLQEIFATEMNKIDESFFQRLDDSFLVPDDKRDSKYPIFGNLAQEKTYHDEF |
| | PTIYHLRKYLADSSKKADLRLVYLALAHMIKYRGHFLIEGDFNSRNNDIKKNFQNFLDSYNAIFESD |
| | LSLENSEQLEEIVKDKISKSAKRERVLKLFPDEKSTGLFSEFLKLIVGNQADFKKHFDLEEKAPLQFS |
| | KDTYDEDLENLLGQIGDDFADLFLVAKKLYDAILLSGILTVTDPSTKAPLSASMIERYENHQKDLA |
| | ALKQFIQNNLQEKYDEVFSDQSKDGYAGYINGKTTQEAFYKYIKNLLSKFEGADYFLDKIEREDFL |
| | RKQRTFDNGSIPHQIHLQEMNAILRRQGEHYPFLKENREKIEKILTFRIPYYVGPLARGNRDFAWLT |
| | RNSDQAIRPWNFEEIVDQASSAEEFINKMTNYDLYLPEEKVLPKHSLLYETFAVYNELTKVKFIAEG |
| | LRDYQFLDSGQKKQIVNQLFKEKRKVTEKDIIHYLHNVDGYDGIELKGIEKQFNASLSTYHDLLKII |
| | KDKEFMDDPKNEEILENIVHTLTIFEDREMIKQRLAQYDSLFDEKVIKALTRRHYTGWGKLSAKLIN |
| | GICDKQTGDTILDYLIDDDKINRNFMQLINDDGLSFKEIIQKAQVVGKTDDLKQVVQELPGSPAIKK |
| | GILQSIKIVDELVKVMGYEPEQIVIEMARENQTTNQGRRNSQQRYKLIEDGVKNLASDLNGNILKE |
| | YPTDNQALQNDRLFLYYLQNGKDMYTGEALDINQLSNYDIDHIVPQAFIKDDSLDNRVLTSSKDN |
| | RGKSDNVPSLEVVQKRKAFWQQLLDSKLISERKFNNLTKAERGGLDERDKVGFIKRQLVETRQITK |
| | HVAQILDARFNTEVDESGKRIRKVKIITLKSNLVSNFRKEFGLYKVREINDYHHAHDAYLNAVVAK |
| | AILKKYPKLEPEFVYGDYQKYDLKRYISKSKNPKEVDKATQKYFFYSNLLNFFKEEVHYADGTIIK |
| | RENIEYSKDTGEIAWNKEKDFATVRKVLSCPQVNIVKKTEVQTGGFSKESILPKRNSDKLIARKKD |
| | WDPKKYGGFDSPTVAYSVLVVAKVEKGKSKKLKSVKELLGITIMERSSFEKNPIDFLEAKGYKEVR |
| | KDLIVKLPKYSLFELENGRKRMLASAGELQKGNELALPSKYVNFLYLASHYEKLKGSPEDNEQKQ |
| | LFVEQHKHYLDEIIEQISEFSKRVILADANLDKVLSAYNKHRDKPIREQAENIIHLFTLTNLGAPAAF |
| | KYFDTTIDRKRYTSTKEVLDATFIHQSITGLYETRIDLSQLGGD |
| 84 | MTKPYSIGLDIGTNSVGWAVITDSYKVPSKKMKVLGNTSKKYVKKNLLGALLFESGITAEGRRLK |
| | RTARRRYTRRRNRILYLQEIFSTEMAKIDESFFQRLDDSFLVPDDKRDSKYPIFGNLVKEKTYHDEF |
| | PTIYHLRKYLADSSKKADLRLVYLALAHMIKYRGHFLIEGDFNSRNNDIKKNFQNFLDSYNAIFESD |
| | LSLENSKQLEEIVKDKISKSAKRERVLKLFPDEKSTGLFSEFLKLIVGNQADFKKHFDLEEKAPLQFS |
| | KDTYDEDLENLLGQIGDDFADLFLVAKKLYDAILLSGILTVTDPSTKAPLSASMIERYENHQKDLA |
| | ALKQFIKNNLPEKYDEVFSDQSKDGYAGYIDGKTTQEAFYKYIKNLLSKFEGADYFLEKIEREDFLR |
| | KQRTFDNGSIPHQIHLQEMNAILRHQGEYYPFLKENKEKIEKILTFRIPYYVGPLARGNRDFAWLTR |
| | NSDQAIRPWNFEEIVDKASSAEDFINKMTNYDLYLPEEKVLPKHSLLYETFAVYNELTKVKFIAEGL |
| | RDYQFLDSGQKKQIVNQLFKEKRKVTEKDIIHYLHNVDGYDGIELKGIEKQFNASLSTYHDLLKIIK |
| | DKEFMDDPKNEEILENIVHTLTIFEDREMIKQRLAQYDSVFDEKVIKALARRHYTGWGKLSAKLIN |
| | GIYDKQSKKTILDYLIDDDKINRNFMQLINDDGLSFKDIIQKAQVVGKTDDVKQVVQELPGSPAIKK |
| | GILQSIKIVDELVKVMGSDPESIVIEMARENQTTNQGRRNSQQRYKKIKEAIKNLDSELNSKILKEYP |
| | TNNQALQNDRLFLYYLQNGKDMYTGEPLDINQLSSYDIDHIVPQAFIKDDSLDNRVLTSSKDNRGK |
| | SDNVPSIEIVERMKGFWQQLLNSKLISERKFNNLTKAERGGLDERDKVGFIKRQLVETRQITKHVA |
| | QILDARFNTEVDSKGKKIRKVKIITLKSNLVSNFRKEFGLYKVREINDYHHAHDAYLNAVVAKAIL |
| | KKYPKLEPEFVYGDYQKYDLKRYISKSKNPKSIDKATQKYFFYSNLLNFFKEEVHYADGTIIKRENI |
| | EYSKDTGEIAWNKEKDFATVRKVLSCPQVNIVKKTEVQTGGFSKESILPKRNSDKLIARKKDWDPK |
| | KYGGFDSPTVAYSVLVVAKVEKGKSKKLKSVKELLGITIMERSSFEKNPIDFLEAKGYKEVRKDLI |
| | VKLPKYSLFELENGRKRMLASAGELQKGNELALPSKYVNFLYLASHYEKLKGSPEDNEQKQLFVE |
| | QHKHYLDEIIEQISEFSKRVILADANLDKVLSAYNKHRDKPIREQAENIIHLFTLTNLGAPAAFKYFD |
| | TTIDRKRYTSTKEVLDATFIHQSITGLYETRIDLSQLGGD |
| 85 | MTKPYSIGLDIGTNSVGWAVITDSYKVPSKKMKVLGNTSKKYVKKNLLGALLFESGITAEGRRLK |
| | RTARRRYTRRRNRILYLQEIFSTEMSKIDESFFQRLDDSFLVPDDKRDSKYPIFGNLVEEKVYHDEFP |
| | TIYHLRKYLADSSKKADLRLVYLALAHMIKYRGHFLIEGDFNSRNNDIKKNFQDFLDSYNAIFESDL |
| | SLENSKQLEEIVKDKISKSAKKDRLLKLFPGEKNSGIFSEFLKLIVGNQADFRKYFNLDEKASLHFSK |
| | ESYDEDLETLLGHIGDDYSDVFLKAKKLYDAILLSGILTVTDNETEAPLSSAMIMRYKEHEEDLALL |
| | KAYIRKISLETYNEVFKDDTKNGYAGYIDGKTNQEDFYVYLKKLLTGLEGADYFLEKINREDLLRK |
| | QRTFDNGSIPYQIHLQEMRAILDKQAKFYPFLAKNKERIEKILTFRIPYYVGPLARGNSDFAWSIRKR |
| | NEKITPWNFEDVIDKESSAEAFINRMTSFDLYLPEEKVLPKHSLLYETFTVYNELTKVRFIAESMRD |
| | YQFLDSKQKKDIVRLYFKGKRKVTDKDIIDYLHAIDGYDGIELKGIEKQFNASLSTYHDLLKIIKDK |
| | EFLDDSENEAIIEEIIHTLTIFEDREMIKQRLSKYEDVFSKSVLKKLSRRHYTGWGKLSAKLINGIRDE |
| | KSGKTILDYLIDDGVSNRNFMQLIHDDTLSFKKKIQKAQIIGDQDNIKQVVQSLPGSPAIKKGILQSI |
| | KIVDELVKVMGRNPESIVVEMARENQYTNQGKSNSQQRLKRLEESLKELGSKILKENVPAKLSKID |
| | NNALQNERLYLYYLQNGKDMYTGEELDIDRLSDYDIDHIIPQAFIKDNSIDNKVLVSSASNRGKSD |
| | DVPSLDVVKKRKGFWHQLLKSKLISQRKFDNLTKAERGGLTDDDKAGFIKRQLVETRQITKHVAQ |
| | ILDARFNVKRDDDNKVIRDVKIVLLKSSLVSQFRKDFELYKVREINDFHHAHDAYLNAVVASALLK |
| | KYPKLEPEFVYGDYSKSHISKEQDKATAKYFFYSNLLNFFKSDQKYLTDGTVVFRENIEYSKDTGEI |
| | AWNKEKDFATVRKVLSCPQVNIVKKTEVQTGGFSKESILPKRNSDKLIARKKDWDPKKYGGFDSP |
| | TVAYSVLVVAKVEKGKSKKLKSVKELLGITIMERSSFEKNPIDFLEAKGYKEVRKDLIVKLPKYSLF |
| | ELENGRKRMLASAGELQKGNELALPSKYVNFLYLASHYEKLKGSPEDNEQKQLFVEQHKHYLDEI |
| | IEQISEFSKRVILADANLDKVLSAYNKHRDKPIREQAENIIHLFTLTNLGAPAAFKYFDTTIDRKRYT |
| | STKEVLDATFIHQSITGLYETRIDLSQLGGD |
| 86 | MTKPYSIGLDIGTNSVGWAVITDSYKVPSKKMKVLGNTSKKYVKKNLLGALLFESGITAEGRRLK |
| | RTARRRYTRRRNRILYLQEIFAAEMNKIDESFFHRLDDSFLVPEDKRGSKYPIFGNLEEEKAYHEKF |
| | PTIYHLRKYLADSSKKADLRLVYLALAHMIKYRGHFLIEGDFNSRNNDIKKSFQNFLDSYNAIFESD |
| | LSLEQNAQVEEIVKDKISKSAKRERVLKLFPDEKSTGLFSEFLKLIVGNQADFKKHFDLEEKAPLQF |
| | SKDTYDEDLENLLGQIGDDFADLFLVAKKLYDAILLSGILTVTDPSTKAPLSASMIERYENHQKDLA |
| | ALKQFIQNNLQEKYDEVFSDQSKDGYAGYINGKTTQEAFYKYIKNLLSKLEGADYFLDKIEREDFL |
| | RKQRTFDNGSIPHQIHLQEMNAILRRQGEYYPFLKENREKIEKILTFRIPYYVGPLARGNRDFAWLT |
| | RNSDQAIRPWNFEEVVDQASSAEDFINKMTNYDLYLPEEKVLPKHSLLYETFAVYNELTKVKFIAE |
| | GLRDYQFLDSGQKKQIVNQLFKEKRKVTEKDIIHYLHNVDGYDGIELKGIEKQFNASLSTYHDLLKI |
| | IKDKEFMDDPKNEEILENIVHTLTIFEDREMIKQRLAQYDSLFDEKVIKALTRRHYTGWGKLSAKLI |
| | NGICDKQTGDTILDFLIDDDKINRNFMQLINDDGLSFKDIIQKAQVVGKTDDVKQVVQELPGSPAIK |
| | KGILQSIKIVDELVKVMGHAPESIVIEMARENQTTNQGKRNSQQRYKRIEEAIKNLAPELDGNILKE |
| | YPTDNQALQNDRLFLYYLQNGKDMYTGEELDINQLSNYDIDHIVPQAFIKDDSLDNRVLTSSKDNR |
| | GKSDNVPSLEVVQKRKAFWQQLLDSKLISERKYNNLTKAERERGGLNELDKVGFIKRQLVETRQIT |
| | KHVARILDERFNEEVDENKRIRKVKIITLKSNLVSNFRKEFELYKVREINDYHHAHDAYLNAVVAK |
| | AILKKYPKLEPEFVYGDYQKYDIKRYISRSKDSKEIDKATEKYFFYSNLLNFFKEEVHYADGTVVFR |
| | RNIEYSKDTGEIAWNKEKDFATVRKVLSCPQVNIVKKTEVQTGGFSKESILPKRNSDKLIARKKDW |
| | DPKKYGGFDSPTVAYSVLVVAKVEKGKSKKLKSVKELLGITIMERSSFEKNPIDFLEAKGYKEVRK |
| | DLIVKLPKYSLFELENGRKRMLASAGELQKGNELALPSKYVNFLYLASHYEKLKGSPEDNEQKQLF |
| | VEQHKHYLDEIIEQISEFSKRVILADANLDKVLSAYNKHRDKPIREQAENIIHLFTLTNLGAPAAFKY |
| | FDTTIDRKRYTSTKEVLDATFIHQSITGLYETRIDLSQLGGD |
| 87 | MKKPYSIGLDIGTNSVGWAVVTDDYKVPAKKMKVLGNTNKESIKKNLIGALLFDAGNTAADRRL |
| | KRTARRRYTRRRNRILYLQEIFAEEMNKVDESFFHRLDDSFLVPEDKRGSKYPIFATLEEEKEYHKK |
| | FPTIYHLRKHLADSKEKADLRLIYLALAHMIKYRGHFLYEESFDIKNNDIQKIFNEFISIYDNTFEGSS |
| | LSGQNAQVEAIFTDKISKSAKRERVLKLFPDEKSTGLFSEFLKLIVGNQADFKKHFDLEEKAPLQFS |
| | KDTYDEDLENLLGQIGDDFADLFLVAKKLYDAILLSGILTVTDPSTKAPLSASMIERYENHQKDLA |
| | ALKQFIQNNLQEKYDEVFSDQSKDGYAGYIDGKTTQEAFYKYIKNLLSKLEGADYFLDKIEREDFL |
| | RKQRTFDNGSIPHQIHLQEMNAIIRRQGEHYPFLKENKEKIEKILTFRIPYYVGPLARGNRDFAWLTR |
| | NSDQAIRPWNFEEIVDQASSAEDFINKMTNYDLYLPEEKVLPKHSLLYETFAVYNELTKVKFIAEG |
| | MRDYQFLDSGQKKKIVSQLFEDKRKVTEKDIIHYLHTIDGYDGIELSGIEKQFNASLSTYHDLLKIIK |
| | DKAFLDDSENEAIIEEIIHTLTIFEDREMIKQRLAQYADIFDKSVLKKLARRHYTGWGKLSAKLINGI |
| | RDKQSNKTILDYLIDDGKINRNFMQLINDDSLSFKETIQKAQVVGDTDDVKQVVQELPGSPAIKKGI |
| | LQSIKIVDELVKVMGHNPDNIVIEMARENQTTNRGRSQSQQRLKKLQDSLKELGSNILNEEKPSYIE |
| | DKVENSHLQNDQLFLYYIQNGKDMYTGEELDIDHLSDYDIDHIIPQAFIKDDSIDNRVLTSSAKNRG |
| | KSDDVPSLDIVRERKADWIRLYKSKLISKRKFDNLTKAERGGLTEADKAGFIKRQLVETRQITKHV |
| | AQILDSRFNTKRDENDKVIRDVKVITLKSNLVSQFRKDFELYKVREINDYHHAHDAYLNAVVGTA |
| | LLKKYPKLAPEFVYGEYKKYDIRKFITSSKDKATAKYFFYSNLMNFFKRIIRYSNGKIVVRPVVEYS |
| | KDTGEIAWNKEKDFATVRKVLSCPQVNIVKKTEVQTGGFSKESILPKRNSDKLIARKKDWDPKKY |
| | GGFDSPTVAYSVLVVAKVEKGKSKKLKSVKELLGITIMERSSFEKNPIDFLEAKGYKEVRKDLIVKL |
| | PKYSLFELENGRKRMLASAGELQKGNELALPSKYVNFLYLASHYEKLKGSPEDNEQKQLFVEQHK |
| | HYLDEIIEQISEFSKRVILADANLDKVLSAYNKHRDKPIREQAENIIHLFTLTNLGAPAAFKYFDTTID |
| | RKRYTSTKEVLDATFIHQSITGLYETRIDLSQLGGD |
| 88 | MTKPYSIGLDIGTNSVGWAVITDSYKVPSKKMKVLGNTSKKYVKKNLLGALLFESGITAEGRRLK |
| | RTARRRYTRRRNRILYLQEIFSTEMAKVDESFFQRLDDSFLVPDDKRDSKYPIFGNLVEEKTYHDEF |
| | PTIYHLRKYLADSQKKADLRLVYLALAHMIKYRGHFLIEGDFNSRNNDIKKNFQNFLDSYNAIFES |
| | DLSLENSKQLEEIVKDKISKSAKRERVLKLFPDEKSTGLFSEFLKLIVGNQAEFKKHFDLEEKAPLQF |
| | SKDTYDEDLENLLGQIGDDFADLFLVAKKLYDAILLSGILTVTDPSTKAPLSASMIERYENHQKDLA |
| | ALKQFIKANLPEKYDEVFSDESKDGYAGYINGKTTQEAFYKYIKNLLSKFEGADYFLEKIEREDFLR |
| | KQRTFDNGSIPHQIHLQEMNAILRRQGEHYPFLKENKEKIQQILTFRIPYYVGPLARGNRDFAWLTR |
| | NSDQAIRPWNFEEIVDQASSAEDFINKMTNYDLYLPEEKVLPKHSLLYETFAVYNELTKVKFIAEGL |
| | RDYQFLDSGQKKQIVNQLFKEKRKVTEKDIIQFLHNVDGYDGIELKGIEKQFNASLSTYHDLLKIIK |
| | DKEFMDDPKNEEILENIVHTLTIFEDREMIKQRLAQYDSLFDEKVIKALTRRHYTGWGKLSAKLING |
| | IRDKQTSKTILDYLIDDGKINRNFMQLINDDGLSFKEIIQKAQVVGKTDDVKQVVQELPGSPAIKKGI |
| | LQSIKIVDELVKVMGHAPESIVIEMARENQTTARGKRNSQQRYKKIEEAVKNLASELDGNILKEYPT |
| | DNAQLQNERLFLYYLQNGRDMYTGKALDINNLSNYDIDHIVPQAFIKDDSIDNRVLTSSKDNRGKS |
| | DNVPSIEIVEKMKGFWQQLLNSKLISERKYNNLTKAERERGGLNELDKVGFIKRQLVETRQITKHV |
| | AQILDARFNTEVNEKNQKIRKVKIITLKSNLVSNFRKEFGLYKVREINDYHHAHDAYLNAVVAKAI |
| | LKKYPKLEPEFVYGDYQKYDIKRYISRSKDPKDIEKATEKYFFYSNLLNFFKEEVHYADGTIVKREN |
| | IEYSKDTGEIAWNKEKDFATVRKVLSCPQVNIVKKTEVQTGGFSKESILPKRNSDKLIARKKDWDP |
| | KKYGGFDSPTVAYSVLVVAKVEKGKSKKLKSVKELLGITIMERSSFEKNPIDFLEAKGYKEVRKDL |
| | IVKLPKYSLFELENGRKRMLASAGELQKGNELALPSKYVNFLYLASHYEKLKGSPEDNEQKQLFVE |
| | QHKHYLDEIIEQISEFSKRVILADANLDKVLSAYNKHRDKPIREQAENIIHLFTLTNLGAPAAFKYFD |
| | TTIDRKRYTSTKEVLDATFIHQSITGLYETRIDLSQLGGD |
| 89 | MTKPYSIGLDIGTNSVGWAVITDSYKVPSKKMKVLGNTSKKYVKKNLLGALLFESGITAEGRRLK |
| | RTARRRYTRRRNRILYLQEIFSTEMAKIDESFFQRLDDSFLVPDDKRDSKYPIFGNLVEEKTYHDEFP |
| | TIYHLRKYLADSTKKADLRLVYLALAHMIKYRGHFLIEGDFNSRNNDIKKNFQDFLDSYNAIFESD |
| | LSLENSKQLEEIVKDKISKSAKRERVLKLFPDEKSTGLFSEFLKLIVGNQADFKKHFDLEEKAPLQFS |
| | KDTYDEDLENLLGQIGDDFADLFLVAKKLYDAILLSGILTVTDPSTKAPLSASMIERYENHQKDLA |
| | ALKQFIQNNLQEKYDEVFSDQSKDGYAGYIDGKTTQEAFYKYIKNLLSKLEGADYFLDKIEREDFL |
| | RKQRTFDNGSIPHQIHLQEMNAILRRQGEHYPFLKENKEKIEKILTFRIPYYVGPLARGNRDFAWLT |
| | RNSDQAIRPWNFEEIVDQASSAEDFINKMTNYDLYLPEEKVLPKHSLLYETFAVYNELTKVKFIAEG |
| | LRDYQFLDSGQKKQIVNQLFKDKRKVTEKDIIHYLHTVDGYDGIELKGIEKQFNASLSTYHDLLKII |
| | KDKEFMDDPKNEEILENIVHTLTIFEDREMIKQRLAQYDSLFDEKVIKALTRRHYTGWGKLSAKLIN |
| | GIYDKQSKKTILDYLIDDDKINRNFMQLINDDGLSFKEIIQKAQVVGKTDDVKQVVQELPGSPAIKK |
| | GILQSIKIVDELVKVMGHAPESIVIEMARENQTTNQGKRNSQQRYKRIEEAIKNLSIDLNSKILKEYP |
| | TDNQALQNDRLFLYYLQNGKDMYTGKELDINQLSNYDIDHIVPQAFIKDDSLDNRVLTSSKDNRG |
| | KSDNVPSIEVVQKRKAFWQQLLDSKLISQRKFDNLTKAERGGLTEDDKAGFIKRQLVETRQITKHV |
| | AQILDARFNTEVDENGKRIRKVKIITLKSNLVSNFRKEFGLYKVREINDYHHAHDAYLNAVVAKAI |
| | LKKYPKLEPEFVYGDYQKYDIKRYISRSKDPKDVEKATQKYFFYSNLLNFFKEEVHYADGTIIKRE |
| | NIEYSKDTGEIAWNKEKDFATVRKVLSCPQVNIVKKTEVQTGGFSKESILPKRNSDKLIARKKDWD |
| | PKKYGGFDSPTVAYSVLVVAKVEKGKSKKLKSVKELLGITIMERSSFEKNPIDFLEAKGYKEVRKD |
| | LIVKLPKYSLFELENGRKRMLASAGELQKGNELALPSKYVNFLYLASHYEKLKGSPEDNEQKQLFV |
| | EQHKHYLDEIIEQISEFSKRVILADANLDKVLSAYNKHRDKPIREQAENIIHLFTLTNLGAPAAFKYF |
| | DTTIDRKRYTSTKEVLDATFIHQSITGLYETRIDLSQLGGD |
| 90 | MTKPYSIGLDIGTNSVGWAVITDSYKVPSKKMKVLGNTSKKYVKKNLLGALLFESGITAEGRRLK |
| | RTARRRYTRRRNRILYLQEIFSTEMAKIDESFFQRLDDSFLVPDDKRDSKYPIFGNLVEEKVYHDEF |
| | PTIYHLRKYLADSSKKADLRLVYLALAHMIKYRGHFLIEGDFNSRNNDIKKNFQNFLDSYNAIFESD |
| | LSLENSKQLEEIVKDKISKSAKRERVLKLFPDEKSTGLFSEFLKLIVGNQADFKKHFDLEEKAPLQFS |
| | KDTYDEDLENLLGQIGDDFADLFLVAKKLYDAILLSGILTVTDPSTKAPLSASMIERYENHQKDLA |
| | ALKQFVKANLPEKYDEVFSDQSKDGYAGYIDGKTTQEAFYKYIKNLISKIEGADYFLEKIEREDFLR |
| | KQRTFDNGSIPHQIHLQEMHAILHNQGEYYPFLKDNKEKIEKILTFRIPYYVGPLARGNRDFAWLTR |
| | NSDQAIRPWNFEEIVDKASSAEDFINKMTNYDLYLPEEKVLPKHSLLYETFAVYNELTKVKFIAEGL |
| | RDYQFLDSGQKKQIVNQLFKDKRKVTEKDIIQFLHNVDGYDGIELKGIEKQFNASLSTYHDLLKIIK |
| | DKEFMDDPKNEEILENIIHTLTIFEDREMIKQRLAQYDSLFDEKVIKALTRRHYTGWGKLSAKLINGI |
| | YDKQSRKTILDYLIDDDKINRNFMQLINDDGLSFKDIIQKAQVVGKTDDVKQVVQELPGSPAIKKGI |
| | LQSIKIVDELVKVMGHAPESIVIEMARENQTTARGKKNSQQRYKRIEDALKNLAPGLDSNILKENPT |
| | DNIQLQNDRLFLYYLQNGKDMYTGEALDINQLSNYDIDHIIPQAFIKDDSLDNRVLTSSKDNRGKS |
| | DNVPSIEVVQKRKAFWQQLLDSKLISERKENNLTKAERGGLDERDKVGFIKRQLVETRQITKHVAQ |
| | ILDARFNTEVDDNNKKIRKVKIITLKSNLVSNFRKEFGLYKVREINDYHHAHDAYLNAVVAKAILK |
| | KYPKLEPEFVYGDYQKYDIKRYISRSKDPKDIEKATEKYFFYSNLLNFFKEEVHYADGTIIKRDKVE |
| | YSNDTGEIAWNKEKDFATVRKVLSCPQVNIVKKTEVQTGGFSKESILPKRNSDKLIARKKDWDPK |
| | KYGGFDSPTVAYSVLVVAKVEKGKSKKLKSVKELLGITIMERSSFEKNPIDFLEAKGYKEVRKDLI |
| | VKLPKYSLFELENGRKRMLASAGELQKGNELALPSKYVNFLYLASHYEKLKGSPEDNEQKQLFVE |
| | QHKHYLDEIIEQISEFSKRVILADANLDKVLSAYNKHRDKPIREQAENIIHLFTLTNLGAPAAFKYFD |
| | TTIDRKRYTSTKEVLDATFIHQSITGLYETRIDLSQLGGD |
| 91 | MTKPYSIGLDIGTNSVGWAVITDDYKVPSKKMKVLGNTSKKYVKKNLLGALLFESGITAEGRRLK |
| | RTARRRYTRRRNRILYLQEIFSTEMNKIDEGFFQRLDDSFLVPDDKRDSKYPIFGNLVEEKTYHDEF |
| | PTIYHLRKYLADSSKKADLRLVYLALAHMIKYRGHFLIEGDFNSRNNDIKKNFQDFLDSYNAIFESD |
| | LSLENSKQLEEIVKDKISKSAKRERVLKLFPDEKSTGLFSEFLKLIVGNQADFKKHFDLEEKAPLQFS |
| | KDTYDEDLENLLGQIGDDFADLFLVAKKLYDAILLSGILTVTDPSTKAPLSASMIERYENHQNDLSA |
| | LKQFIKRNLSEKYDEVFSDESKDGYAGYINGKTTQEAFYKYIKNLISKIEGADYFLEKIEREDFLRK |
| | QRTFDNGSIPHQIHLQEMNAILRHQGEYYPFLQENKEKIEKILTFRIPYYVGPLARGNRDFAWLTRN |
| | SDQAIRPWNFEEIVDQASSAEDFINKMTNYDLYLPEEKVLPKHSLLYETFAVYNELTKVKFIAEGLR |
| | DYQFLDSGQKKQIVNQLFKEKRKVTEKDIIQFLHNVDGYDGIELKGIEKQFNASLSTYHDLLKIIKD |
| | KEFMDDPKNEEILENIIHTLTIFEDREMIKQRLAQYDSLFDEKVIKALTRRHYTGWGKLSAKLINGIC |
| | DKKTGKTILDYLIDDGEINRNFMQLIHDDGLSFKDIIQKAQVVGKTDDVKQVVQELPGSPAIKKGIL |
| | QSIKIVDELVKVMGHAPESIVIEMARENQTTARGKKNSQQRYKRIEDALKNLAPGLDSNILKENPT |
| | DNIQLQNDRLFLYYLQNGKDMYTGEPLDINQLSNYDIDHIVPQAFIKDDSLDNRVLTSSKDNRGKS |
| | DNVPSIEVVQKRKAFWQQLLDSKLISERKENNLTKAERGGLDERDKVGFIKRQLVETRQITKHVAQ |
| | ILDSRENTEVNDQGKKIRKVKIITLKSNLVSNFRKEFGLYKVREINDYHHAHDAYLNAVVAKAILK |
| | KYPKLEPEFVYGDYQKYDIKRYISKSRNPKTIDKATQKYFFYSNLLNFFKEEVHYADGTIVKRENV |
| | EYSNDTGEIAWNKEKDFATVRKVLSCPQVNIVKKTEVQTGGFSKESILPKRNSDKLIARKKDWDPK |
| | KYGGFDSPTVAYSVLVVAKVEKGKSKKLKSVKELLGITIMERSSFEKNPIDFLEAKGYKEVRKDLI |
| | VKLPKYSLFELENGRKRMLASAGELQKGNELALPSKYVNFLYLASHYEKLKGSPEDNEQKQLFVE |
| | QHKHYLDEIIEQISEFSKRVILADANLDKVLSAYNKHRDKPIREQAENIIHLFTLTNLGAPAAFKYFD |
| | TTIDRKRYTSTKEVLDATFIHQSITGLYETRIDLSQLGGD |
| 92 | MTKPYSIGLDIGTNSVGWAVITDSYKVPSKKMKVLGNTSKKYVKKNLLGALLFESGITAEGRRLK |
| | RTARRRYTRRRNRILYLQEIFSTEMAKIDESFFQRLDDSFLVPDDKRDSKYPIFGNLAKEKTYHDEF |
| | PTIYHLRKYLADSQKKADLRLVYLALAHMIKYRGHFLIEGDFNSRNNDIKKNFQKFLDSYNAIFES |
| | DLSLENSKQLEEIVKDKISKSAKRERVLKLFPDEKSTGLFSEFLKLIVGNQAEFKKHFDLEEKAPLQF |
| | SKDTYDEDLENLLGQIGDGFADLFLVAKKLYDAILLSGILTVTDPSTKAPLSASMIERYENHQKDLA |
| | ALKQFIQNNLQEKYDEVFSDQSKDGYAGYIDGKTTQEAFYKYIKNTLSKIEGSDYFLDKIEREDFLR |
| | KQRTFDNGSIPHQIHLQEMNAIIRRQGEHYPFLKENKEKIEKILTFRIPYYVGPLARGNRDFAWLTR |
| | NSDQAIRPWNFEEVVDKASSAEDFINKMTNYDLYLPEEKVLPKHSLLYETFAVYNELTKVKFIAEG |
| | LRDYQFLDSGQKKQIVNQLFKEKRKVTEKDIIHYLHTVDGYDGIELKGIEKQFNASLSTYHDLLKII |
| | KDKEFMDDPKNEEILENIVHTLTIFEDREMIKQRLAQYDSLFDEKVIKALTRRHYTGWGKLSAKLIN |
| | GIRDKKSGKTILDYLIDDGKINRNFMQLIHDDGLSFKEIIQKAQVVGKTDDVKQVVQELPGSPAIKK |
| | GILQSIKIVDELVKVMGHAPESIVIEMARENQTTNRGKRNSQQRYKRIENAVKNLASELNGNILKEY |
| | PTNNQALQNERLFLYYLQNGKDMYTGESLDINQLSSYDIDHIVPQAFIKDDSLDNRVLTSSKDNRG |
| | KSDNVPSLEIVEKMKGFWQQLLDSKLISERKENNLTKAERGGLDERDKVGFIKRQLVETRQITKHV |
| | AQILDARFNTEVNENNQKIRKVKIITLKSNLVSNFRKEFGLYKVREINDYHHAHDAYLNAVVAKAI |
| | LKKYPKLEPEFVYGDYQKYDVKRYISRSKDPKEIEKATEKYFFYSNLLNFFKEEVHYADGTIIKREN |
| | VEYSNDTGEIAWNKEKDFATVRKVLSCPQVNIVKKTEVQTGGFSKESILPKRNSDKLIARKKDWDP |
| | KKYGGFDSPTVAYSVLVVAKVEKGKSKKLKSVKELLGITIMERSSFEKNPIDFLEAKGYKEVRKDL |
| | IVKLPKYSLFELENGRKRMLASAGELQKGNELALPSKYVNFLYLASHYEKLKGSPEDNEQKQLFVE |
| | QHKHYLDEIIEQISEFSKRVILADANLDKVLSAYNKHRDKPIREQAENIIHLFTLTNLGAPAAFKYFD |
| | TTIDRKRYTSTKEVLDATFIHQSITGLYETRIDLSQLGGD |
| 93 | MNKPYSIGLDIGTNSVGWAVITDSYKVPSKKMKVLGNTSKKYVKKNLLGALLFESGITAEGRRLK |
| | RTARRRYTRRRNRILYLQEIFATEMAKIDESFFQRLDDSFLVPDDKRDSKYPIFGNLVEEKTYHDEF |
| | PTIYHLRKYLADSQKKADLRLVYLALAHMIKYRGHFLIEGDFNSRNNDIKKNFQDFLDSYNAIFES |
| | DLSLENNAQVEEIVKDKISKSAKRERVLKLFPDEKSTGLFSEFLKLIVGNQADFKKHFDLEEKAPLQ |
| | FSKDTYDEDLENLLGQIGDDFADLFLVAKKLYDAILLSGILTVTDPSTKAPLSASMIERYENHQKDL |
| | AALKQFVKANLPEKYDEVFSDQSKDGYAGYIDGKTTQEAFYKYIKNLLSKFEGADYFLEKIEREDF |
| | LRKQRTFDNGSIPHQIHLQEMNAIIRRQGEHYPFLKENKEKIEKILTFRIPYYVGPLARGNRDFAWLT |
| | RNSDQAIRPWNFEEIVDQASSAEDFINKMTNYDLYLPEEKVLPKHSLLYETFAVYNELTKVKFIAEG |
| | LRDYQFLDSGQKKQIVNQLFKEKRKVTEKDIIHYLHTVDGYDGIELKGIEKQFNASLSTYHDLLKII |
| | KDKEFMDDPKNEEILENIVHTLTIFEDREMIKQRLAQYDSLFDEKVIKALTRRHYTGWGKLSAKLIN |
| | GISDKQTGNTILDFLIDDKKINRNFMQLIHDDGLSFKEIIQKAQVVGKTDDVKQVVQELPGSPAIKK |
| | GILQSIKIVDELVKVMGHDPEHIVVEMARENQFTNQGRRNSQQRYKKIENAIKNLDSNLNSKILKE |
| | YPTNNQALQNDRLFLYYLQNGKDMYTGEALDINQLSNYDIDHIVPQAFIKDDSLDNRVLTSSKDN |
| | RGKSDNVPSIEVVEKMKAFWQQLLDSKLISERKYNNLTKAERERGGLNELDKVGFIKRQLVETRQI |
| | TKHVAQILDARFNTEVNEKGKKIRKVKIITLKSNLVSNFRKEFGLYKVREINDYHHAHDAYLNAVV |
| | AKAILKKYPKLEPEFVYGDYQKYDIKRYISRSKDPKDVEKATEKYFFYSNLLNFFKEEVHYADGTI |
| | VKRENIEYSKDTGEIAWNKEKDFATVRKVLSCPQVNIVKKTEVQTGGFSKESILPKRNSDKLIARK |
| | KDWDPKKYGGFDSPTVAYSVLVVAKVEKGKSKKLKSVKELLGITIMERSSFEKNPIDFLEAKGYKE |
| | VRKDLIVKLPKYSLFELENGRKRMLASAGELQKGNELALPSKYVNFLYLASHYEKLKGSPEDNEQ |
| | KQLFVEQHKHYLDEIIEQISEFSKRVILADANLDKVLSAYNKHRDKPIREQAENIIHLFTLTNLGAPA |
| | AFKYFDTTIDRKRYTSTKEVLDATFIHQSITGLYETRIDLSQLGGD |
| 94 | MTKPYSIGLDIGTNSVGWAVITDSYKVPSKKMKVLGNTSKKYVKKNLLGALLFESGITAEGRRLK |
| | RTARRRYTRRRNRILYLQEIFATEMAKIDESFFQRLDDSFLVPDDKRDSKYPIFGNLVKEKTYHDEF |
| | PTIYHLRKYLADSSKKADLRLVYLALAHMIKYRGHFLIEGDFNSRNNDIKKNFQNFLDSYNAIFESD |
| | LSLENSKQLEEIVKDKISKSAKRERVLKLFPDEKSTGLFSEFLKLIVGNQADFKKHFDLEEKAPLQFS |
| | KDTYDEDLENLLGQIGDDFADLFLVAKKLYDAILLSGILTVTDPSTKAPLSASMIERYENHQKDLAT |
| | LKQFIKNNLSEKYDEVFSDQSKDGYAGYIDGKTTQEAFYKYIKNTLSKIEGSDYFLDKIEREDFLRK |
| | QRTFDNGSIPHQIHLQEMNAILHRQGEHYPFLKENKEKIEQILTFRIPYYVGPLARGNRDFAWLTRN |
| | SDQAIRPWNFEEVVDKASSAEDFINKMTNYDLYLPEEKVLPKHSLLYETFAVYNELTKVKFIAEGL |
| | RDYQFLDSGQKKQIVNQLFKEKRKVTEKDIIHYLHNVDGYDGIELKGIEKQFNASLSTYHDLLKIIK |
| | DKEFMDDPKNEEILENIVHTLTIFEDREMIKQRLAQYDSLFDEKVIKALTRRHYTGWGKLSAKLING |
| | IYDKQSRKTILDYLIDDDKINRNFMQLIHDDGLSFKEIIQKAQVVGKTDDVKQVVQELPGSPAIKKG |
| | ILQSIKIVDELVKVMGHAPESIVIEMARENQTTARGKKNSQQRYKRIEEALKNLAPGLDSNILKEHP |
| | TDNIQLQNDRLFLYYLQNGRDMYTGKPLDINQLSNYDIDHIVPQAFIKDDSIDNRVLTSSKDNRGKS |
| | DNVPSLEVVQKRKAFWQQLLDSKLISERKENNLTKAERGGLDERDKVGFIKRQLVETRQITKHVA |
| | QILDARFNTEVNDKKQKIRKVKIITLKSNLVSNFRKEFGLYKVREINDYHHAHDAYLNAVVAKAIL |
| | KKYPKLEPEFVYGDYQKYDLKRYISKSKDPKEIEKATQKYFFYSNLLNFFKEEVHYADGTIIKRENI |
| | EYSKDTGEIAWNKEKDFATVRKVLSCPQVNIVKKTEVQTGGFSKESILPKRNSDKLIARKKDWDPK |
| | KYGGFDSPTVAYSVLVVAKVEKGKSKKLKSVKELLGITIMERSSFEKNPIDFLEAKGYKEVRKDLI |
| | VKLPKYSLFELENGRKRMLASAGELQKGNELALPSKYVNFLYLASHYEKLKGSPEDNEQKQLFVE |
| | QHKHYLDEIIEQISEFSKRVILADANLDKVLSAYNKHRDKPIREQAENIIHLFTLTNLGAPAAFKYFD |
| | TTIDRKRYTSTKEVLDATFIHQSITGLYETRIDLSQLGGD |
| 95 | MTKPYSIGLDIGTNSVGWAVITDSYKVPSKKMKVLGNTSKKYVKKNLLGALLFESGITAEGRRLK |
| | RTARRRYTRRRNRILYLQEIFSTEMSKIDEGFFQRLDDSFLVPDDKRDSKYPIFGNLVEEKVYHDEF |
| | PTIYHLRKYLADSSKKADLRLVYLALAHMIKYRGHFLIEGDFNSRNNDIKKNFQDFLDSYNAIFESD |
| | LSLENSKQLEEIVKDKISKSAKRERVLKLFPDEKSTGLFSEFLKLIVGNQADFKKHFDLEEKAPLQFS |
| | KDTYDEDLENLLGQIGDDFADLFLVAKKLYDAILLSGILTVTDPSTKAPLSASMIQRYEDHQEDLK |
| | KLKEFIRRNIPEKYDEVFADSSKNGYAGYIDGKTTQEEFYKYIKEKLNNIKGADYFLDKIEREDFLR |
| | KQRTFDNGSIPHQIHLQEMNAIIRRQGEHYPFLKENKEKIEKILTFRIPYYVGPLARGNRDFAWLTR |
| | NSDQAIRPWNFEEVVDQASSAEDFINKMTNYDLYLPEEKVLPKHSLLYETFAVYNELTKVKFIAEG |
| | LRDYQFLDSGQKKQIVNQLFKEKRKVTEKDIIQFLHNVDGYDGIELKGIEKQFNASLSTYHDLLKII |
| | KDKEFMDDPKNEEILENIVHTLTIFEDREMIKQRLAQYDSLFDEKVIKALTRRHYTGWGKLSAKLIN |
| | GICDKKTGKTILDYLIDDGKINRNFMQLIHDDGLSFKEIIQKAQVVGKTDDVKQVVQELPGSPAIKK |
| | GILQSIKIVDELVKVMGHAPESIVIEMARENQTTNQGKRNSQQRYKRIEDGVKNLASDLNGNILKE |
| | YPTDNQALQNDRLFLYYLQNGKDMYTGEELDINQLSNYDIDHIVPQAFIKDDSLDNRVLTSSKDNR |
| | GKSDNVPSLEVVQKRKAFWQQLLDSKLISERKFNNLTKAERGGLDERDKVGFIKRQLVETRQITKH |
| | VAQILDARFNTEKDENGKKIRKVKIITLKSNLVSNFRKEFGLYKVREINDYHHAHDAYLNAVVAK |
| | AILKKYPKLEPEFVYGDYQKYDVKRYISRSKDPKEIEKATQKYFFYSNLLNFFKEEVHYADGTIIKR |
| | ENIEYSNDTGEIAWNKEKDFATVRKVLSCPQVNIVKKTEVQTGGFSKESILPKRNSDKLIARKKDW |
| | DPKKYGGFDSPTVAYSVLVVAKVEKGKSKKLKSVKELLGITIMERSSFEKNPIDFLEAKGYKEVRK |
| | DLIVKLPKYSLFELENGRKRMLASAGELQKGNELALPSKYVNFLYLASHYEKLKGSPEDNEQKQLF |
| | VEQHKHYLDEIIEQISEFSKRVILADANLDKVLSAYNKHRDKPIREQAENIIHLFTLTNLGAPAAFKY |
| | FDTTIDRKRYTSTKEVLDATFIHQSITGLYETRIDLSQLGGD |
| 96 | MTKPYSIGLDIGTNSVGWAVITDSYKVPSKKMKVLGNTSKKYVKKNLLGALLFESGITAEGRRLK |
| | RTARRRYTRRRNRILYLQEIFSTEMAKIDESFFQRLDDSFLVPDDKRDSKYPIFGNLVEEKTYHDEFP |
| | TIYHLRKYLADSQKKADLRLVYLALAHMIKYRGHFLIEGDFNSRNNDIKKNFQNFLDSYNAIFESD |
| | LSLENSKQLEEIVKDKISKSAKRERVLKLFPDEKSTGLFSEFLKLIVGNQADFKKHFNLEEKAPLHFS |
| | KDTYDEDLENLLGQIGDDFADLFLVAKKLYDAILLSGILTVTDPSTKAPLSASMIDRYENHQKDLA |
| | ALKQFIQNNLQEKYDEVFSDESKDGYAGYIDGKTTQEAFYKYIKNLISKFEGADYFLEKIEREDFLR |
| | KQRTFDNGSIPHQIHLQEMNAILHHQGEYYPFLKENSEKIQQILTFRIPYYVGPLARGNRDFAWLTR |
| | NSDQAIRPWNFEEIVDKASSAEDFINKMTNYDLYLPEEKVLPKHSLLYETFAVYNELTKVKFIAEGL |
| | RDYQFLDSGQKKQIVNQLFKEKRKVTEKDIIHYLHNVDGYDGIELKGIEKQFNASLSTYHDLLKIIK |
| | DKEFMDDPKNEEILENIVHTLTIFEDREMIKQRLAQYDSLFDEKVIKALTRRHYTGWGKLSAKLING |
| | ISDKQTGNTILDYLIDDDKINRNFMQLIHDDGLSFKEIIQKAQVVGKTDDVKQVVQELPGSPAIKKG |
| | ILQSIKIVDELVKVMGHAPESIVIEMARENQTTNQGRRNSQQRYKLIEDGVKNLASDLNGNILKEYP |
| | TNNQALQNDRLFLYYLQNGKDMYTGESLDINQLSSYDIDHIVPQAFIKDDSLDNRVLTSSKDNRGK |
| | SDNVPSLEVVQKRKAFWQQLLDSKLISERKENNLTKAERGGLDERDKVGFIRRQLVETRQITKHVA |
| | QILDARFNVEVNDKKQKIRNVKIITLKSNLVSNFRKEFGLYKVREINDYHHAHDAYLNAVVAKAIL |
| | KKYPKLEPEFVYGDYQKYDIKRYISRSKDPKEIEKATEKYFFYSNLLNFFKEEVHYADGTIIKRENIE |
| | YSKDTGEIAWNKEKDFATVRKVLSCPQVNIVKKTEVQTGGFSKESILPKRNSDKLIARKKDWDPK |
| | KYGGFDSPTVAYSVLVVAKVEKGKSKKLKSVKELLGITIMERSSFEKNPIDFLEAKGYKEVRKDLI |
| | VKLPKYSLFELENGRKRMLASAGELQKGNELALPSKYVNFLYLASHYEKLKGSPEDNEQKQLFVE |
| | QHKHYLDEIIEQISEFSKRVILADANLDKVLSAYNKHRDKPIREQAENIIHLFTLTNLGAPAAFKYFD |
| | TTIDRKRYTSTKEVLDATFIHQSITGLYETRIDLSQLGGD |
| 97 | MTKPYSIGLDIGTNSVGWAVITDDYKVPSKKMKVLGNTSKKYVKKNLLGALLFESGITAEGRRLK |
| | RTARRRYTRRRNRILYLQEIFSTEMNKIDESFFQRLDDSFLVPDDKRDSKYPIFGNLVEEKTYHDEFP |
| | TIYHLRKYLADSQKKADLRLVYLALAHMIKYRGHFLIEGDFNSRNNDIKKNFQNFLDSYNAIFESD |
| | LSLENSKQLEEIVKDKISKSAKRERVLKLFPDEKSTGLFSEFLKLIVGNQADFKKHFDLEEKAPLQFS |
| | KDTYDEDLENLLGQIGDDYADLFLVAKKLYDAILLSGILTATNPITKAPLSASMIERYDKHHKDLV |
| | LLKELVRRNLPEKYDEVFSDQSKDGYAGYIDGKTTQEAFYKYIKNTLNKIEGADYFLDKIEREDFL |
| | RKQRTFDNGSIPHQIHLQEMNAILRRQGEHYPFLKENKEKIQQILTFRIPYYVGPLARGNRDFAWLT |
| | RNSDQAIRPWNFEEIVDKASSAEDFINKMTNYDLYLPEEKVLPKHSLLYETFAVYNELTKVKFIAEG |
| | LRDYQFLDSGQKKQIVNQLFKEKRKVTEKDIIQFLHNVDGYDGIELKGIEKQFNASLSTYHDLLKII |
| | KDKEFMDDPKNEEILENIVHTLTIFEDREMIKQRLAQYDSLFDEKVIKALTRRHYTGWGKLSAKLIN |
| | GIYDKQSKKTILDYLIDDDKINRNFMQLINDDGLSFKEIIQKAQVVGKTDDVKQVVQELPGSPAIKK |
| | GILQSIKIVDELVKVMGHDPESIVIEMARENQTTNQGRRNSQQRYKKIENAIKNLDSELNSKILKEYP |
| | TDNQALQNERLFLYYLQNGKDMYTDEELDINQLSNYDIDHIVPQAFIKDDSLDNRVLTSSKDNRGK |
| | SDNVPSIEVVQKRKAFWQQLLDSKLISERKENNLTKAERGGLDERDKVGFIKRQLVETRQITKHVA |
| | QILDARFNTEVNEKKQKIRTVKIITLKSNLVSNFRKEFGLYKVREINDYHHAHDAYLNAVVAKAIL |
| | KKYPKLEPEFVYGDYQKYDIKRYISKSKDPKDIEKATEKYFFYSNLLNFFKEEVHYADGTIIKRENIE |
| | YSKDTGEIAWNKEKDFATVRKVLSCPQVNIVKKTEVQTGGFSKESILPKRNSDKLIARKKDWDPK |
| | KYGGFDSPTVAYSVLVVAKVEKGKSKKLKSVKELLGITIMERSSFEKNPIDFLEAKGYKEVRKDLI |
| | VKLPKYSLFELENGRKRMLASAGELQKGNELALPSKYVNFLYLASHYEKLKGSPEDNEQKQLFVE |
| | QHKHYLDEIIEQISEFSKRVILADANLDKVLSAYNKHRDKPIREQAENIIHLFTLTNLGAPAAFKYFD |
| | TTIDRKRYTSTKEVLDATFIHQSITGLYETRIDLSQLGGD |
| 98 | MTKPYSIGLDIGTNSVGWAVITDSYKVPSKKMKVLGNTSKKYVKKNLLGALLFESGITAEGRRLK |
| | RTARRRYTRRRNRILYLQEIFSTEMAKIDESFFQRLDDSFLVPDDKRDSKYPIFGNLVEEKTYHDEFP |
| | TIYHLRKYLADSSKKADLRLVYLALAHMIKYRGHFLIEGDFNSRNNDIKKNFQDFLDSYNAIFESDL |
| | SLENSKQLEEIVKDKISKSAKKDRLLKLFPGEKNSGIFSEFLKLIVGNQADFRKYFNLDEKASLHESK |
| | ESYDEDLETLLGHIGDDYSDVFLKAKKLYDAILLSGILTVTDNETEAPLSSAMIMRYKEHEGDLALL |
| | KAYIRNISLETYNEVFKDDTKNGYAGYIDGKTNQEDFYVYLKKLLTGLEGADYFLEKINREDLLRK |
| | QRTFDNGSIPYQIHLQEMRAIIDKQAKFYPFLAKNKERIEKILTFRIPYYVGPLARGNSDFAWSIRKR |
| | NEKITPWNFEDVIDKESSAEAFINRMTSFDLYLPEEKVLPKHSLLYETFTVYNELTKVRFIAESMRD |
| | YQFLDSKQKKDIVRLYFKGKRKVTDKDIIEYLHAIDGYDGIELKGIEKQFNSSLATYHDLLNIINDK |
| | EFLDDSSNEAIIEEIIHTLTIFEDREMIKQRLSKFENIFDKSVLKKLSRRHYTGWGKLSAKLINGIRDE |
| | KSGKTILDYLIDDGISNRNFMQLIHDDTLSFKKKIQKAQIIGDQDNIKQVVQSLPGSPAIKKGILQSIK |
| | IVDELVKVMGRNPESIVVEMARENQYTNQGKSNSQQRLKRLEEAIKELGSNVLKKENPVLKETKID |
| | NQALQNERLYLYYLQNGKDMYTGDDLDIDRLSNYDIDHIIPQAFLKDNSIDNKVLVSSASNRGKSD |
| | DVPSLDVVKKRKTFWHQLLKSKLISQRKFDNLTKAERGGLTPEDKAGFIRRQLVETRQITKHVARL |
| | LDERFNVKRDDNDKLIRNVKIITLKSSLVSQFRKDFELYKVREINDFHHAHDAYLNAVVAKAILTK |
| | YPKLEPEFVYGDYPKYNSFRERKSATQKVFFYSNIMNLFKKEVRLADGTIVVRPVIEYSKDTGEVA |
| | WNKEKDFATVRKVLSCPQVNIVKKTEVQTGGFSKESILPKRNSDKLIARKKDWDPKKYGGFDSPT |
| | VAYSVLVVAKVEKGKSKKLKSVKELLGITIMERSSFEKNPIDFLEAKGYKEVRKDLIVKLPKYSLFE |
| | LENGRKRMLASAGELQKGNELALPSKYVNFLYLASHYEKLKGSPEDNEQKQLFVEQHKHYLDEII |
| | EQISEFSKRVILADANLDKVLSAYNKHRDKPIREQAENIIHLFTLTNLGAPAAFKYFDTTIDRKRYTS |
| | TKEVLDATFIHQSITGLYETRIDLSQLGGD |
| 99 | MTKPYSIGLDIGTNSVGWAVITDSYKVPSKKMKVLGNTSKKYVKKNLLGALLFESGITAEGRRLK |
| | RTARRRYTRRRNRILYLQEIFAAEMNKIDESFFQRLDDSFLVPDDKRDSKYPIFGNLVEEKAYHDEF |
| | PTIYHLRKYLADSSKKADLRLVYLALAHMIKYRGHFLIEGDFNSRNNDIKKNFQEFLDSYNAIFESD |
| | LSLENSKQLEEIVKDKISKSAKRERVLKLFPDEKSTGLFSEFLKLIVGNQADFKKHFDLEEKAPLQFS |
| | KDTYDEDLENLLGQIGDDFADLFLVAKKLYDAILLSGILTVTDPSTKAPLSASMIERYENHQKDLAL |
| | LKQFIKQNLSQEKYDEVFKDESKNGYAGYIENGVKQDEFYKYLKNTLSKIEGSDYFLDKIEREDFL |
| | RKQRTFDNGSIPHQIHLQEMNAIIRRQGEHYPFLKENKEKIEKILTFRIPYYVGPLARGNRDFAWLTR |
| | NSDQAIRPWNFEEVVDQASSAEDFINKMTNYDLYLPEEKVLPKHSLLYETFAVYNELTKVKFIAEG |
| | LRDYQFLDSGQKKQIVNQLFKEKRKVTEKDIIQFLHNVDGYDGIELKGIEKQFNASLSTYHDLLKII |
| | KDKEFMDDPKNEEILENIVHTLTIFEDREMIKQRLAQYDSLFDEKVIKALTRRHYTGWGKLSAKLIN |
| | GICDKKTGKTILDYLIDDGEINRNFMQLINDDGLSFKEIIQKAQVVGKTDDVKQVVQELPGSPAIKK |
| | GILQSIKIVDELVKVMGHAPESIVIEMARENQTTARGKKNSQQRYKRIEDALKNLAPGLDSNILKEN |
| | PTDNIQLQNDRLFLYYLQNGKDMYTGEPLDINQLSSYDIDHIVPQAFIKDDSLDNRVLTSSKDNRG |
| | KSDNVPSIEVVQKRKAFWQQLLDSKLISERKENNLTKAERGGLDERDKVGFIKRQLVETRQITKHV |
| | AQILDARFNTEVNEKGQKIRKVKIITLKSNLVSNFRKEFGLYKVREINDYHHAHDAYLNAVVAKAI |
| | LKKYPKLEPEFVYGDYQKYDIKRYISRSKDPKDVEKATQKYFFYSNLLNFFKEEVHYADGTIIKRE |
| | NIEYSKDTGEVAWNKEKDFATVRKVLSCPQVNIVKKTEVQTGGFSKESILPKRNSDKLIARKKDW |
| | DPKKYGGFDSPTVAYSVLVVAKVEKGKSKKLKSVKELLGITIMERSSFEKNPIDFLEAKGYKEVRK |
| | DLIVKLPKYSLFELENGRKRMLASAGELQKGNELALPSKYVNFLYLASHYEKLKGSPEDNEQKQLF |
| | VEQHKHYLDEIIEQISEFSKRVILADANLDKVLSAYNKHRDKPIREQAENIIHLFTLTNLGAPAAFKY |
| | FDTTIDRKRYTSTKEVLDATFIHQSITGLYETRIDLSQLGGD |
| 100 | MKKPYSIGLDIGTNSVGWAVVTDDYKVPAKKMKVLGNTDKSHIKKNLLGALLFDSGETAEGRRL |
| | KRTARRRYTRRRNRILYLQEIFATEMAKVDESFFQRLDDSFLVPDDKRDSKYPIFGNLAQEKTYHD |
| | EFPTIYHLRKYLADSQKKADLRLVYLALAHMIKYRGHFLIEGDFNSRNNDIKKNFQDFLDSYNAIFE |
| | SDLSLENSKQLEEIVKDKISKSAKRERVLKLFPDEKSTGLFSEFLKLIVGNQADFKKHFDLEEKAPLQ |
| | FSKDTYDEDLENLLGQIGDDFADLFLVAKKLYDAILLSGILTVTDPSTKAPLSASMIERYENHQKDL |
| | AALKQFIQNNLQEKYDEVFSDQSKDGYAGYINGKTTQEAFYKYIKNLLSKLEGSDYFLDKIEREDF |
| | LRKQRTFDNGSIPHQIHLQEMNAIIRRQGEHYPFLQENKEKIEKILTFRIPYYVGPLARGNRDFAWLT |
| | RNSDQAIRPWNFEEVVDKASSAEDFINKMTNYDLYLPEEKVLPKHSLLYETFAVYNELTKVKFIAE |
| | GLRDYQFLDSGQKKQIVNQLFKEKRKVTEKDIIHFLHNVDGYDGIELKGIEKQFNASLSTYHDLLKI |
| | IKDKEFMDDPKNEEILENIVHTLTIFEDREMIKQRLAQYDTLFDEKVIKALTRRHYTGWGKLSAKLI |
| | NGICDKQTGDTILDFLIDDDKINRNFMQLINDDGLSFKEIIQKAQVVGKTDDVKQVVQELPGSPAIK |
| | KGILQSIKIVDELVKVMGHNPESIVIEMARENQTTNQGRRNSQQRYKKIEEAIKNLDSNLNSKILKE |
| | YPTENQALQNDRLFLYYLQNGKDMYTDEELDINQLSNYDIDHIVPQAFIKDDSLDNRVLTSSKDNR |
| | GKSDNVPSIEIVQKRKGFWQQLLDSKLISERKENNLTKAERGGLDERDKVGFIKRQLVETRQITKH |
| | VAQILDARFNTEVNEKKQKIRTVKIITLKSNLVSNFRKEFGLYKVREINDYHHAHDAYLNAVVAKA |
| | ILKKYPKLEPEFVYGDYQKYDLKRYISRSKDPKDVEKATQKYFFYSNLLNFFKEEVHYADGTIIKRE |
| | NIEYSKDTGEIAWNKEKDFATVRKVLSCPQVNIVKKTEVQTGGFSKESILPKRNSDKLIARKKDWD |
| | PKKYGGFDSPTVAYSVLVVAKVEKGKSKKLKSVKELLGITIMERSSFEKNPIDFLEAKGYKEVRKD |
| | LIVKLPKYSLFELENGRKRMLASAGELQKGNELALPSKYVNFLYLASHYEKLKGSPEDNEQKQLFV |
| | EQHKHYLDEIIEQISEFSKRVILADANLDKVLSAYNKHRDKPIREQAENIIHLFTLTNLGAPAAFKYF |
| | DTTIDRKRYTSTKEVLDATFIHQSITGLYETRIDLSQLGGD |
| 101 | MTKPYSIGLDIGTNSVGWAVITDSYKVPSKKMKVLGNTSKKYVKKNLLGALLFESGITAEGRRLK |
| | RTARRRYTRRRNRILYLQEIFSTEMAKIDESFFQRLDDSFLVPDDKRDSKYPIFGNLVKEKTYHDEF |
| | PTIYHLRKYLADSSKKADLRLVYLALAHMIKYRGHFLIEGDFNSRNNDIKKNFQNFLDSYNAIFESD |
| | LSLENSKQLEEIVKDKISKSAKRERVLKLFPDEKSTGLFSEFLKLIVGNQADFKKHFDLEEKAPLQFS |
| | KDTYDEDLENLLGQIGDDFADLFLVAKKLYDAILLSGILTVTDPSTKAPLSASMIERYENHQKDLA |
| | ALKQFIKNNLSEKYDEVFSDQSKDGYAGYIDGKTTQEAFYKYIKNLLSKFEGADYFLEKIEREDFLR |
| | KQRTFDNGSIPHQIHLQEMNAILRRQGEHYPFLKENKEKIEKILTFRIPYYVGPLARGNRDFAWLTR |
| | NSDQAIRPWNFEEVVDKASSAEDFINKMTNYDLYLPEEKVLPKHSLLYETFAVYNELTKVKFIAEG |
| | LRDYQFLDSGQKKQIVNQLFKEKRKVTEKDIIHYLHNVDGYDGIELKGIEKQFNASLSTYHDLLKII |
| | KDKEFMDDPKNEEILENIVHTLTIFEDREMIKQRLAQYDSLFDEKVIKALTRRHYTGWGKLSAKLIN |
| | GIYDKQSKKTILDYLIDDDKINRNFMQLIHDDGLSFKEIIQKAQVVGKTDDVKQVVQELPGSPAIKK |
| | GILQSIKIVDELVKVMGHAPESIVIEMARENQTTNQGRRNSQQRYKLIEDGVKNLASDLNGNILKEY |
| | PTNNQALQNERLFLYYLQNGKDMYTGEALDINQLSNYDIDHIIPQAFIKDDSLDNRVLTSSKDNRG |
| | KSDNVPSIEVVQKRKAFWQQLLDSKLISERKENNLTKAERGGLDERDKVGFIKRQLVETRQITKHV |
| | AQILDARFNTEVDETGKKIRKVKIITLKSNLVSNFRKEFGLYKVREINDYHHAHDAYLNAVVAKAI |
| | LKKYPKLEPEFVYGDYQKYDIKRYISKSKDPKDIEKATEKYFFYSNLLNFFKEKVTYADGTITKREN |
| | IEYSKDTGEIAWNKEKDFATVRKVLSCPQVNIVKKTEVQTGGFSKESILPKRNSDKLIARKKDWDP |
| | KKYGGFDSPTVAYSVLVVAKVEKGKSKKLKSVKELLGITIMERSSFEKNPIDFLEAKGYKEVRKDL |
| | IVKLPKYSLFELENGRKRMLASAGELQKGNELALPSKYVNFLYLASHYEKLKGSPEDNEQKQLFVE |
| | QHKHYLDEIIEQISEFSKRVILADANLDKVLSAYNKHRDKPIREQAENIIHLFTLTNLGAPAAFKYFD |
| | TTIDRKRYTSTKEVLDATFIHQSITGLYETRIDLSQLGGD |
| 102 | MNKPYSIGLDIGTNSVGWAVITDNYKVPSKKMKVLGNTSKKYVKKNLLGALLFESGITAEGRRLK |
| | RTARRRYTRRRNRILYLQEIFIDGMNEIDESFFQRLDDSFLVPDDKRDSKYPIFGNLVEEKTYHDEFP |
| | TIYHLRKYLADSSKKADLRLVYLALAHMIKYRGHFLIEGDFNSRNNDIKKNFQEFLDSYNAIFESDL |
| | SLENSKQLEEIVKDKISKSAKRERVLKLFPDEKSTGLFSEFLKLIVGNQAEFKKHFDLEEKAPLQFSK |
| | DTYDEDLENLLGQIGDDFADLFLVAKKLYDAILLSGILTVTDPSTKAPLSASMIERYENHQKDLAAL |
| | KQFIQNNLQEKYDEVFSDQSKDGYAGYIDGKTTQEAFYKYIKNLLSKLEGADYFLDKIEREDFLRK |
| | QRTFDNGSIPHQIHLQEMNAILRRQGEHYPFLKENKEKIEKILTFRIPYYVGPLARGNRDFAWLTRN |
| | SDQAIRPWNFEEIVDKASSAEDFINKMTNYDLYLPEEKVLPKHSLLYETFAVYNELTKVKFIAEGLR |
| | DYQFLDSGQKKQIVNQLFKEKRKVTEKDIIQYLHNVDGYDGIELKGIEKQFNASLSTYHDLLKIIKD |
| | KEFMDDPKNEEILENIVHTLTIFEDREMIKQRLAQYDSLFDEKVIKALTRRHYTGWGKLSAKLIDGI |
| | YDKQSRKTILDYLIDDDKINRNFMQLINDDGLSFKEIIQKAQVVGKTDDVKQVVQELPGSPAIKKGI |
| | LQSIKIVDELVKVMGHDPESIVIEMARENQTTNQGRRNSQQRYKKIENAIKNLDSNLNSKILKEYPT |
| | NNQALQNDRLFLYYLQNGKDMYKDQELDINQLSNYDIDHIVPQAFIKDDSLDNRVLTSSKDNRGK |
| | SDNVPSLEVVQKRKGFWQQLLDSKLISERKENNLTKAERGGLDERDKVGFIKRQLVETRQITKHVA |
| | QILDARFNTEVNENNQRIRKVKIITLKSNLVSNFRKEFGLYKVREINDYHHAHDAYLNAVVAKAIL |
| | KKYPKLEPEFVYGDYQKYDVKRYISRSKDPKDIEKATEKYFFYSNLLNFFKEEVHYADGTIVKREN |
| | VEYSNDTGEIAWNKEKDFATVRKVLSCPQVNIVKKTEVQTGGFSKESILPKRNSDKLIARKKDWDP |
| | KKYGGFDSPTVAYSVLVVAKVEKGKSKKLKSVKELLGITIMERSSFEKNPIDFLEAKGYKEVRKDL |
| | IVKLPKYSLFELENGRKRMLASAGELQKGNELALPSKYVNFLYLASHYEKLKGSPEDNEQKQLFVE |
| | QHKHYLDEIIEQISEFSKRVILADANLDKVLSAYNKHRDKPIREQAENIIHLFTLTNLGAPAAFKYFD |
| | TTIDRKRYTSTKEVLDATFIHQSITGLYETRIDLSQLGGD |
| 103 | MNKPYSIGLDIGTNSVGWAVITDSYKVPSKKMKVLGNTSKKYVKKNLLGALLFESGITAEGRRLK |
| | RTARRRYTRRRNRILYLQEIFSTEMNKIDESFFQRLDDSFLVPDDKRDSKYPIFGNLAQEKTYHEEFP |
| | TIYHLRKYLADSQKKADLRLVYLALAHMIKYRGHFLIEGDFNSRNNDIKKNFEKFLDSYNAIFESD |
| | LSLENSAQVEEIVKDKISKSAKRERVLKLFPDEKSTGLFSEFLKLIVGNQADFKKHFDLEEKAPLQFS |
| | KDTYDEDLENLLGQIGDGFADLFLVAKKLYDAILLSGILTVTDPSTKAPLSASMIERYENHQKDLA |
| | ALKQFIKSNLSEKYDEVFSDQSKDGYAGYINGKTTQEAFYKYIKNLLSKFEGADYFLEKIEREDFLR |
| | KQRTFDNGSIPHQIHLQEMHAILHRQGEYYPFLKENKEKIEKILTFRIPYYVGPLARGNRDFAWLTR |
| | NSDQAIRPWNFEEIVDQASSAEDFINKMTNYDLYLPEEKVLPKHSLLYETFAVYNELTKVKFIAEGL |
| | RDYQFLDSGQKKQIVNQLFKEKRKVTEKDIIHYLHNVDGYDGIELKGIEKQFNASLSTYHDLLKIIK |
| | DKEFMDDPKNEEILENIVHTLTIFEDREMIKQRLAQYDSLFDEKVIKALTRRHYTGWGKLSAKLING |
| | ICDKQTGDTILDFLIDDDKINRNFMQLINDDGLSFKEIIQKAQVVGKTDDVKQVVQELPGSPAIKKGI |
| | LQSIKIVDELVKVMGHAPESIVIEMARENQTTNQGRRNSQQRYKKIENAIKNLDSNLNSKILKEYPT |
| | DNQALQNDRLFLYYLQNGKDMYTDEELDINQLSNYDIDHIVPQAFIKDDSLDNRVLTSSKDNRGK |
| | SDNVPSLEVVQKRKAFWQQLLDSKLISERKYNNLTKAERERGGLNELDKVGFIKRQLVETRQITKH |
| | VAQILDARFNTEVNEKKQKIRTVKIITLKSNLVSNFRKEFGLYKVREINDYHHAHDAYLNAVVAKA |
| | ILKKYPKLEPEFVYGDYQKYDIKRYISKSKDPKEIDKATEKYFFYSNLLNFFKEEVHYADGTIIKREN |
| | IEYSKDTGEIAWNKEKDFATVRKVLSCPQVNIVKKTEVQTGGFSKESILPKRNSDKLIARKKDWDP |
| | KKYGGFDSPTVAYSVLVVAKVEKGKSKKLKSVKELLGITIMERSSFEKNPIDFLEAKGYKEVRKDL |
| | IVKLPKYSLFELENGRKRMLASAGELQKGNELALPSKYVNFLYLASHYEKLKGSPEDNEQKQLFVE |
| | QHKHYLDEIIEQISEFSKRVILADANLDKVLSAYNKHRDKPIREQAENIIHLFTLTNLGAPAAFKYFD |
| | TTIDRKRYTSTKEVLDATFIHQSITGLYETRIDLSQLGGD |
| 104 | MTKPYSIGLDIGTNSVGWAVITDSYKVPSKKMKVLGNTSKKYVKKNLLGALLFESGITAEGRRLK |
| | RTARRRYTRRRNRILYLQEIFSTEMAKIDESFFQRLDDSFLVPDDKRDSKYPIFGNLVEEKTYHDEFP |
| | TIYHLRKYLADSSKKADLRLVYLALAHMIKYRGHFLIEGDFNSRNNDIKKNFQEFLDSYNAIFESDL |
| | SLENSKQLEEIVKDKISKSAKRERVLKLFPDEKSTGLFSEFLKLIVGNQADFKKHFDLEEKAPLQFSK |
| | DTYDEDLENLLGQIGDGFADLFLVAKKLYDAILLSGILTVTDPSTKAPLSASMIERYENHQKDLAAL |
| | KQFIKSNLSEKYDEVFSDQSKDGYAGYIDGKTTQEAFYKYIKNLLSKFEGADYFLDKIEREDFLRK |
| | QRTFDNGSIPHQIHLQEMNAIIRRQGEHYPFLKENKEKIEKILTFRIPYYVGPLARGNRDFAWLTRNS |
| | DQAIRPWNFEEIVDQASSAEDFINKMTNYDLYLPEEKVLPKHSLLYETFAVYNELTKVKFIAEGLR |
| | DYQFLDSGQKKQIVNQLFKEKRKVTEKDIIHYLHNVDGYDGIELKGIEKQFNASLSTYHDLLKIIKD |
| | KAFMDDAKNEEILENIVHTLTIFEDREMIKQRLAQYDSLFDEKVIKALTRRHYTGWGKLSAKLINGI |
| | CDKQTGNTILDYLIDDDKINRNFMQLINDDGLSFKEIIQKAQVVGKTDDLKQVVQNLPGSPAIKKGI |
| | LQSIKLVDELVKVMGSDPESIVIEMARENQTTNQGRRNSQQRYKLLEDGVKNLASDLNGNILKEYP |
| | TDNQALQNERLFLYYLQNGKDMYTGEALDINQLSSCDIDHIIPQAFIKDDSLDNRVLTSSKDNRGK |
| | SDNVPSIEIVQKMKGFWQQLLDSKLISERKENNLTKAERGGLDERDKVGFIRRQLVETRQITKHVA |
| | QILDARFNTEVNEKKQKIRKVKIITLKSNLVSNFRKEFGLYKVREINDYHHAHDAYLNAVVAKAIL |
| | KKYPKLEPEFVYGDYQKYDIKRYISRSKDPKTIEKATQKYFFYSNLLNFFKEEVHYADGTIVKRENI |
| | EYSKDTGEIAWNKEKDFATVRKVLSCPQVNIVKKTEVQTGGFSKESILPKRNSDKLIARKKDWDPK |
| | KYGGFDSPTVAYSVLVVAKVEKGKSKKLKSVKELLGITIMERSSFEKNPIDFLEAKGYKEVRKDLI |
| | VKLPKYSLFELENGRKRMLASAGELQKGNELALPSKYVNFLYLASHYEKLKGSPEDNEQKQLFVE |
| | QHKHYLDEIIEQISEFSKRVILADANLDKVLSAYNKHRDKPIREQAENIIHLFTLTNLGAPAAFKYFD |
| | TTIDRKRYTSTKEVLDATFIHQSITGLYETRIDLSQLGGD |
| 105 | MTKPYSIGLDIGTNSVGWAVITDSYKVPSKKMKVLGNTSKKYVKKNLLGALLFESGITAEGRRLK |
| | RTARRRYTRRRNRILYLQEIFSTEMAKIDESFFQRLDDSFLVPDDKRDSKYPIFGNLVEEKTYHDEFP |
| | TIYHLRKYLADSTKKADLRLVYLALAHMIKYRGHFLIEGEFNSKNNDIKKNFQNFLDSYNAIFESDL |
| | SLENSAQVEEIVKDKISKSAKRERVLKLFPDEKSTGLFSEFLKLIVGNQADFKKHFDLEEKAPLQFS |
| | KDTYDEDLENLLGQIGDDFADLFLVAKKLYDAILLSGILTVTDPSTKAPLSASMIERYENHQKDLA |
| | ALKQFIQNNLQEKYDEVFSDQSKDGYAGYIDGKTTQEAFYKYIKNLLSKLEGADYFLDKIEREDFL |
| | RKQRTFDNGSIPHQIHLQEMNAIIRRQGEHYPFLQENKEKIEKILTFRIPYYVGPLARGNRDFAWLTR |
| | NSDQAIRPWNFEEIVDKASSAEDFINKMTNYDLYLPEEKVLPKHSLLYETFAVYNELTKVKFIAEGL |
| | RDYQFLDSGQKKQIVNQLFKEKRKVTEKDIIHFLHNVDGYDGIELKGIEKQFNASLSTYHDLLKIIK |
| | DKEFMDDPKNQEILENIVHTLTIFEDREMIKQRLAQYDSLFDEKVIKALTRRHYTGWGKLSAKLIN |
| | GICDKQTGKTILDYLIDDGEINRNFMQLIHDDGLSFKEIIQKAQVVGKTDDVKQVVQELPGSPAIKK |
| | GILQSIKIVDELVKVMGHAPESIVIEMARENQTTARGKKNSQQRYKRIEDAIKNLAPELDSKILKEYP |
| | TDNQALQNDRLFLYYLQNGKDMYTGNPLDINRLSDYDIDHIIPQAFIKDDSIDNRVLTSSKDNRGKS |
| | DNVPSLEVVEKMKGFWQQLLKSKLISQRKFDNLTKAERGGLTEHDKAGFIKRQLVETRQITKHVA |
| | QILDARFNTEVNDKKQKIRNVKIITLKSNLVSNFRKEFGLYKVREINDYHHAHDAYLNAVVAKAIL |
| | KKYPKLEPEFVYGDYQKYDIKRYISRSKDPKDIEKATEKYFFYSNLLNFFKEEVHYADGTIVKRENI |
| | EYSKDTGEIAWNKEKDFATVRKVLSCPQVNIVKKTEVQTGGFSKESILPKRNSDKLIARKKDWDPK |
| | KYGGFDSPTVAYSVLVVAKVEKGKSKKLKSVKELLGITIMERSSFEKNPIDFLEAKGYKEVRKDLI |
| | VKLPKYSLFELENGRKRMLASAGELQKGNELALPSKYVNFLYLASHYEKLKGSPEDNEQKQLFVE |
| | QHKHYLDEIIEQISEFSKRVILADANLDKVLSAYNKHRDKPIREQAENIIHLFTLTNLGAPAAFKYFD |
| | TTIDRKRYTSTKEVLDATFIHQSITGLYETRIDLSQLGGD |
| 106 | MTKPYSIGLDIGTNSVGWAVITDSYKVPSKKMKVLGNTSKKYVKKNLLGALLFESGITAEGRRLK |
| | RTARRRYTRRRNRILYLQEIFSTEMAKIDESFFQRLDDSFLVPDDKRDSKYPIFGNLVEEKTYHDEFP |
| | TIYHLRKYLADSSKKADLRLVYLALAHMIKYRGHFLIEGDENSKNNDIKKNYQNFLDSYNAIFESD |
| | LSLENNAQVEEIVKDKISKSAKRERVLKLFPDEKSTGLFSEFLKLIVGNQADFKKHFDLEEKAPLQF |
| | SKDTYDEDLENLLGQIGDDFADLFLVAKKLYDAILLSGILTVTDPSTKAPLSASMIERYENHQKDLA |
| | ALKQFVRSNLPEKYDEVFSDQSKDGYAGYIDGKTTQEAFYKYIKNLLSKFEGSDYFLDKIEREDFL |
| | RKQRTFDNGSIPHQIHLQEMKAIIHRQGEHYPFLQENKEKIEKILTFRIPYYVGPLARGNRDFAWLT |
| | RNSDQAIRPWNFEEIVDKASSAEDFINKMTNYDLYLPEEKVLPKHSLLYETFAVYNELTKVKFIAEG |
| | LRDYQFLDSGQKKQIVNQLFKEKRKVTEKDIIQFLHNVDGYDGIELKGIEKQFNASLSTYHDLLKII |
| | KDKEFMDDPKNEEILENIVHTLTIFEDREMIKQRLAQYDSLFDEKVIKALTRRHYTGWGKLSAKLIN |
| | GIYDKQSKKTILDYLIDDDKINRNFMQLINDDGLSFKEIIQKAQVVGETDDLKQVVQNLPGSPAIKK |
| | GILQSIKIVDELVKVMGSDPESIVIEMARENQTTNQGRRNSQQRYKKIEDAIKNLDSNLNSKILKEYP |
| | TDNQALQNDRLFLYYLQNGKDMYTGEELDINQLSNYDIDHIIPQAFIKDDSLDNRVLTSSKDNRGK |
| | SDNVPSIEVVEKMKGFWQQLLNSKLISEHKFNNLTKAERGGLDERDKVGFIKRQLVETRQITKHVA |
| | QILDARFNTEVNENGKKIRKVKIITLKSNLVSNFRKEFGLYKVREINDYHHAHDAYLNAVVAKAIL |
| | KKYPKLEPEFVYGDYQKYDIKRYISRSKDPKDVEKATQKYFFYSNLLNFFKEEVHYADGTIIKRENI |
| | EYSKDTGEIAWNKEKDFATVRKVLSCPQVNIVKKTEVQTGGFSKESILPKRNSDKLIARKKDWDPK |
| | KYGGFDSPTVAYSVLVVAKVEKGKSKKLKSVKELLGITIMERSSFEKNPIDFLEAKGYKEVRKDLI |
| | VKLPKYSLFELENGRKRMLASAGELQKGNELALPSKYVNFLYLASHYEKLKGSPEDNEQKQLFVE |
| | QHKHYLDEIIEQISEFSKRVILADANLDKVLSAYNKHRDKPIREQAENIIHLFTLTNLGAPAAFKYFD |
| | TTIDRKRYTSTKEVLDATFIHQSITGLYETRIDLSQLGGD |
| 107 | MTKPYSIGLDIGTNSVGWAVITDSYKVPSKKMKVLGNTSKKYVKKNLLGALLFESGITAEGRRLK |
| | RTARRRYTRRRNRILYLQEIFSTEMAKIDESFFQRLDDSFLVPDDKRDSKYPIFGNLVEEKAYHDEF |
| | PTIYHLRKYLADSSKKADLRLVYLALAHMIKYRGHFLIEGDFNSRNNDIKKNFQEFLDSYNAIFESD |
| | LSLENSKQLEEIVKDKISKSAKRERVLKLFPDEKSTGLFSEFLKLIVGNQADFKKHFDLEEKAPLQFS |
| | KDTYDEDLENLLGQIGDGFADLFLVAKKLYDAILLSGILTVTDPSTKAPLSASMIERYENHQKDLA |
| | ALKQFIKANLPEKYDEVFSDQSKDGYAGYIDGKTTQEAFYKYIKNLLSKFEGADYFLDKIEREDFL |
| | RKQRTFDNGSIPHQIHLQEMNAILRRQGEHYPFLKENKEKIEKILTFRIPYYVGPLARGNRDFAWLT |
| | RNSDQAIRPWNFEEIVDQASSAEDFINKMTNYDLYLPEEKVLPKHSLLYETFAVYNELTKVKFIAEG |
| | LRDYQFLDSGQKKQIVNQLFKEKRKVTEKDIIQFLHNVDGYDGIELKGIEKQFNASLSTYHDLLKII |
| | KDKEFMDDPKNEEILENIVHTLTIFEDREMIKQRLAQYDSLFDEKVIKALTRRHYTGWGKLSAKLIN |
| | GIRDKKSGKTILDYLIDDDKINRNFMQLINDDGLSFKEIIQKAQVVGKTDDLKQVVQELPGSPAIKK |
| | GILQSIKIVDELVKVMGHNPENIVVEMARENQFTNQGRRNSQQRYKKIKEAIKNLDSNLNSKILKE |
| | YPTDNQALQNDRLFLYYLQNGKDMYTGEALDINQLSNYDIDHIIPQAFIKDDSLDNRVLTSSKDNR |
| | GKSDNVPSIEVVEKMKAFWQQLLDSKLISERKENNLTKAERERGGLNELDKVGFIKRQLVETRQIT |
| | KHVAQILDARFNTEVNEKNQKIREVKIITLKSNLVSNFRKEFGLYKVREINDYHHAHDAYLNAVVA |
| | KAILKKYPKLEPEFVYGDYQKYDIKRYISRSKDPKEIEKATEKYFFYSNLLNFFKEEVHYADGTIVK |
| | RENIEYSKDTGEIAWNKEKDFATVRKVLSCPQVNIVKKTEVQTGGFSKESILPKRNSDKLIARKKD |
| | WDPKKYGGFDSPTVAYSVLVVAKVEKGKSKKLKSVKELLGITIMERSSFEKNPIDFLEAKGYKEVR |
| | KDLIVKLPKYSLFELENGRKRMLASAGELQKGNELALPSKYVNFLYLASHYEKLKGSPEDNEQKQ |
| | LFVEQHKHYLDEIIEQISEFSKRVILADANLDKVLSAYNKHRDKPIREQAENIIHLFTLTNLGAPAAF |
| | KYFDTTIDRKRYTSTKEVLDATFIHQSITGLYETRIDLSQLGGD |
| 108 | MKKPYSIGLDIGTNSVGWAVVDDDYKVPAKKMRVFGDTDKGHIKKNLLGTLLFDSGETAESRRL |
| | KRTARRRYTRRRNRILYLQEIFAEEMSKVDDSFFHRLEESFLTDDDKNFDSHPIFGNKAEEDAYHQ |
| | KFPTIYHLRKHLADSTEKADLRLVYLALAHMIKFRGHFLIEGQLNAENTDVQTLFNDFVEVYDKTV |
| | EESHLAEITVDALSILTEKVSKSRRLENLIKHYPTEKKNTLFGNLVALALGLQPNFKTNFKLSEDAK |
| | LQFSKDTYEEDLEELLGKIGDDYADLFTSAKNLYDAILLSGILTVDDNSTKAPLSASMIKRYAEHHE |
| | DLEKLKEFIKANKPELYHDIFKDETKNGYAGYIENGVKQDEFYKYLKNTLSKIAGSDYFLDKIERE |
| | DFLRKQRTFDNGSIPHQIHLQEMHAILRRQGDYYPFLKENQAKIEKILTFRIPYYVGPLARKNSRFA |
| | WAEYHSDEPITPWNFDEVVDKEKSAEKFITRMTMNDLYLPEEKVLPKHSHVYETYTVYNELTKIK |
| | YVNEQGESFFFDANMKQEIFDHVFKENRKVSKEKILNYLNKEFPEYRIQDLIGLDKENKSFNASLGT |
| | YHDLKKILDKSFLDDKTNEQIIEDIVLTLTLFEDRDMIRERLQKYSDIFTKQQLKKLERRHYTGWGR |
| | LSYKLINGIRNKENNKTILDFLIDDGRANRNFMQLINDDSLSFKTIIQEAQVVGDVDDIEAVVHDLP |
| | GSPAIKKGILQSVKIVDELVKVMGHNPDNIVIEMARENQTTNRGRSQSQQRLKKLQESLKELGSNIL |
| | NEEKPSYIEDKVENSHLQNDRLFLYYIQNGKDMYTGDELDIDHLSDYDIDHIIPQAFIKDDSIDNRVL |
| | TSSAKNRGKSDDVPSLDIVHDRKADWIRLYKSGLISKRKFDNLTKAERGGLTEADKAGFIKRQLVE |
| | TRQITKHVAQILDGRFNTKRDENDKVIRDVKVITLKSNLVSQFRKDFGFYKVREINDYHHAHDAYL |
| | NAVVGTALLKKYPKLAPEFVYGEYKKYDIRKFITNSSDKATAKYFFYSNLMNFFKRVIRYSNGTVI |
| | VRPVIEYSKDTGEIAWNKEKDFATVRKVLSCPQVNIVKKTEVQTGGFSKESILPKRNSDKLIARKKD |
| | WDPKKYGGFDSPTVAYSVLVVAKVEKGKSKKLKSVKELLGITIMERSSFEKNPIDFLEAKGYKEVR |
| | KDLIVKLPKYSLFELENGRKRMLASAGELQKGNELALPSKYVNFLYLASHYEKLKGSPEDNEQKQ |
| | LFVEQHKHYLDEIIEQISEFSKRVILADANLDKVLSAYNKHRDKPIREQAENIIHLFTLTNLGAPAAF |
| | KYFDTTIDRKRYTSTKEVLDATFIHQSITGLYETRIDLSQLGGD |
| 109 | MTKPYSIGLDIGTNSVGWAVITDSYKVPSKKMKVLGNTSKKYVKKNLLGALLFESGITAEGRRLK |
| | RTARRRYTRRRNRILYLQEIFSTEMSKIDESFFQRLDDSFLVPDDKRDSKYPIFGNLVEEKTYHDEFP |
| | TIYHLRKYLADSSKKADLRLVYLALAHMIKYRGHFLIEGDFNSRNNDIKKNFQDFLDSYNAIFESDL |
| | SLENSKQLEEIVKDKISKSAKKDRILKLFPGEKNSGIFSEFLKLIVGNQADFKKYFNLDDQASLHESK |
| | ESYDEDLETLLGHIGDDYSDVFLKAKKLYDAILLSGILTVTDNETEAPLSSAMIMRYKEHEEDLALL |
| | KAYIRNISLETYNEVFKDDTKNGYAGYIDGKTNQEDFYVYLKKLLTGLEGADYFLEKINREDLLRK |
| | QRTFDNGSIPYQIHLQEMRAIIDKQAKFYPFLAKNKERIEKILTFRIPYYVGPLARGNSDFAWSIRKR |
| | NEKITPWNFEDVIDKESSAEAFINRMTSFDLYLPEEKVLPKHSLLYETFTVYNELTKVRFIAESMRD |
| | YQFLDSKQKKDIVRLYFKGKRKVTDKDIIDYLHAIDGYDGIELKGIEKQFNSSLSTYHDLLNIINDKE |
| | FLDDASNEAIIEEIIHTLTIFEDREMIKQRLSKFENIFDKSVLKKLSRRHYTGWGKLSAKLINGIRDEK |
| | SGKTILDYLIDDGISNRNFMQLIHDDTLSFKKKIQKAQIIGDQDNIKQVVQSLPGSPAIKKGILQSIKI |
| | VDELVKVMGRNPESIVVEMARENQYTNQGKSNSQQRLKRLEESLKELGSKILKENVPAKLSKIDN |
| | NALQNERLYLYYLQNGKDMYTGKDLDIDRLSNYDIDHIIPQAFLKDNSIDNKVLVSSASNRGKSDD |
| | VPSLDVVKKRKGFWHQLLKSKLISQRKFDNLTKAERGGLTEDDKAGFIKRQLVETRQITKHVAQIL |
| | DSRFNTKRDDNDKVIRDVKIITLKSNLVSQFRKEFGLYKVREINDYHHAHDAYLNAVVATAILKKY |
| | PKLEPEFVYGDYQKYDIKKYLASKDDSSLGKATAKMFFYSNLMNFFKKKVRLSDGTVLIRPVIEYS |
| | KDTGEIAWNKEKDFATVRKVLSCPQVNIVKKTEVQTGGFSKESILPKRNSDKLIARKKDWDPKKY |
| | GGFDSPTVAYSVLVVAKVEKGKSKKLKSVKELLGITIMERSSFEKNPIDFLEAKGYKEVRKDLIVKL |
| | PKYSLFELENGRKRMLASAGELQKGNELALPSKYVNFLYLASHYEKLKGSPEDNEQKQLFVEQHK |
| | HYLDEIIEQISEFSKRVILADANLDKVLSAYNKHRDKPIREQAENIIHLFTLTNLGAPAAFKYFDTTID |
| | RKRYTSTKEVLDATFIHQSITGLYETRIDLSQLGGD |
| 110 | MNKPYSIGLDIGTNSVGWAVITDSYKVPSKKMKVLGNTSKKYVKKNLLGALLFESGITAEGRRLK |
| | RTARRRYTRRRNRILYLQEIFSTEMNKIDEGFFQRLDDSFLVPDDKRDSKYPIFGNLVEEKTYHDEF |
| | PTIYHLRKYLADSSKKADLRLVYLALAHMIKYRGHFLIEGDFNSRNNDIKKNFSKFLDSYNAIFESD |
| | LSLENSEQVEEIVKDKISKSAKRERVLKLFPDEKSTGLFSEFLKLIVGNQADFKKHFDLEEKAPLQFS |
| | KDTYEEDLESLLGQIGDIYADLFVVAKKLYDAILLAGILSVKDPSTKAPLSASMIERYDNHQNDLSA |
| | LKQFIRRNLSEKYDEVFSDESKDGYAGYINGKTTQEAFYKYIKNLLSKLEGADYFLDKIEREDFLRK |
| | QRTFDNGSIPHQIHLQEMNAILRRQGEHYPFLKENKEKIEKILTFRIPYYVGPLARGNRDFAWLTRN |
| | SDQAIRPWNFEEIVDQASSAEDFINKMTNYDLYLPEEKVLPKHSLLYETFAVYNELTKVKFIAEGLR |
| | DYQFLDSGQKKQIVNQLFKEKRKVTEKDIIHYLHNVDGYDGIELKGIEKQFNASLSTYHDLLKIIKD |
| | KEFMDDPKNEEILENIVHTLTIFEDREMIKQRLAQYDSLFDEKVIKALTRRHYTGWGKLSAKLINGI |
| | CDKKTGKTILDYLIDDDKINRNFMQLIHDDGLSFKEIIQKAQVVGKTDDVKQVVQELPGSPAIKKGI |
| | LQSIKLVDELVKVMGHAPESIVIEMARENQTTARGKKNSQQRYKRIEDALKNLAPGLDSNILKENP |
| | TDNIQLQNDRLFLYYLQNGKDMYTGEALDINQLSSYDIDHIVPQAFIKDDSLDNRVLTSSKDNRGK |
| | SDNVPSIEVVQKRKAFWQQLLDSKLISERKENNLTKAERGGLDERDKVGFIKRQLVETRQITKHVA |
| | QILDARFNTEVDENGKKIRKVKIITLKSNLVSNFRKEFRLYKVREINDYHHAHDAYLNAVVAKAIL |
| | KKYPKLEPEFVYGDYQKYDIKRYISRSKDPKDIEKATEKYFFYSNLLNFFKEEVHYADGTIVKRENI |
| | EYSKDTGEIAWNKEKDFATVRKVLSCPQVNIVKKTEVQTGGFSKESILPKRNSDKLIARKKDWDPK |
| | KYGGFDSPTVAYSVLVVAKVEKGKSKKLKSVKELLGITIMERSSFEKNPIDFLEAKGYKEVRKDLI |
| | VKLPKYSLFELENGRKRMLASAGELQKGNELALPSKYVNFLYLASHYEKLKGSPEDNEQKQLFVE |
| | QHKHYLDEIIEQISEFSKRVILADANLDKVLSAYNKHRDKPIREQAENIIHLFTLTNLGAPAAFKYFD |
| | TTIDRKRYTSTKEVLDATFIHQSITGLYETRIDLSQLGGD |
| 111 | MTKPYSIGLDIGTNSVGWAVISDSYKVPSKKMKVLGNTSKKYVKKNLLGALLFESGITAEGRRLKR |
| | TARRRYTRRRNRILYLQEIFSTEMAKIDESFFQRLDDSFLVPDDKRDSKYPIFGNLVEEKTYHDKFPT |
| | IYHLRKYLADSSKKADLRLVYLALAHMIKYRGHFLIEGDFNSRNNDIKKNFQNFLDSYNAIFESDLS |
| | LENSKQLEEIVKDKISKSAKRERVLKLFPDEKSTGLFSEFLKLIVGNQADFKKHFDLEEKAPLQFSK |
| | DTYDEDLENLLGQIGDDFADLFLVAKKLYDAILLSGILTVTDPSTKAPLSASMIERYENHQKDLAAL |
| | KQFIQNNLQEKYDEVFSDQSKDGYAGYIDGKTTQEAFYKYIKDLLNNVEGADYFLEKIEREDFLRK |
| | QRTFDNGTIPYQIHLQEMKAILHNQGEYYPFLKENKEKIEKILTFRIPYYVGPLARGNRDFAWLTRN |
| | SDQAIRPWNFEEIVDQASSAEDFINKMTNYDLYLPEEKVLPKHSLLYETFAVYNELTKVKFIAEGLR |
| | DYQFLDSGQKKQIVNQLFKEKRKVTEKDIIQFLHNVDGYDGIELKGIEKQFNASLSTYHDLLKIIKD |
| | KEFMDDPKNEEILENIVHTLTIFEDREMIKQRLAQYDSLFDEKVIKALTRRHYTGWGKLSAKLINGI |
| | YDKQSKKTILDYLIDDGEINRNFMQLINDDGLSFKEIIQKAQVVGKTDDVKQVVQELPGSPAIKKGI |
| | LQSIKLVDELVKVMGDDPESIVIEMARENQTTARGKKNSQQRYKRIEDALKNLAPGLDSNILKEHP |
| | TDNIQLQNDRLFLYYLQNGKDMYTGEALDINQLSNYDIDHIVPQAFIKDDSLDNRVLTSSKDNRGK |
| | SDNVPSIEVVQKRKAFWQQLLDSKLISEHKFNNLTKAERGGLDERDKVGFIKRQLVETRQITKHVA |
| | QILDARFNTEVNDKNQKIRSVKIITLKSNLVSNFRKEFGLYKVREINDYHHAHDAYLNAVVAKAIL |
| | KKYPKLEPEFVYGDYQKYDLKRYISKSKEPKDIEKATEKYFFYSNLLNFFKEEVHYADGTIIKRENI |
| | EYSKDTGEIAWNKEKDFATVRKVLSCPQVNIVKKTEVQTGGFSKESILPKRNSDKLIARKKDWDPK |
| | KYGGFDSPTVAYSVLVVAKVEKGKSKKLKSVKELLGITIMERSSFEKNPIDFLEAKGYKEVRKDLI |
| | VKLPKYSLFELENGRKRMLASAGELQKGNELALPSKYVNFLYLASHYEKLKGSPEDNEQKQLFVE |
| | QHKHYLDEIIEQISEFSKRVILADANLDKVLSAYNKHRDKPIREQAENIIHLFTLTNLGAPAAFKYFD |
| | TTIDRKRYTSTKEVLDATFIHQSITGLYETRIDLSQLGGD |
| 112 | MNKPYSIGLDIGTNSVGWAVITDSYKVPSKKMKVLGNTSKKYVKKNLLGALLFESGITAEGRRLK |
| | RTARRRYTRRRNRILYLQEIFSTEMAKVDESFFQRLDDSFLVPDDKRDSKYPIFGNLVEEKTYHEEF |
| | PTIYHLRKYLADSSKKADLRLVYLALAHMIKYRGHFLIEGDFNSRNNDIKKNFQDFLDSYNAIFESD |
| | LSLENSKQLEEIVKDKISKSAKRERVLKLFPDEKSTGLFSEFLKLIVGNQADFKKHFDLEEKAPLQFS |
| | KDTYDEDLENLLGQIGDDFADLFLVAKKLYDAILLSGILTVTDPNTKAPLSASMIERYENHEEDLA |
| | ALKQFIKNNLPEKYDEVFSDQSKDGYAGYINGKTTQEAFYKYIKNLLSKFEGADYFLDKIEREDFL |
| | RKQRTFDNGSIPHQIHLQEMNAILRRQGEHYPFLKENKEKIEKILTFRIPYYVGPLARGNRDFAWLT |
| | RNSDQAIRPWNFEEVVDKASSAEDFINKMTNYDLYLPEEKVLPKHSLLYETFAVYNELTKVKFIAE |
| | GLRDYQFLDSGQKKQIVNQLFKEKRKVTEKDIIQFLHNVDGYDGIELKGIEKQFNASLSTYHDLLKI |
| | IKDKEFMDDPKNEEILENIVHTLTIFEDREMIKQRLAQYDSLFDEKVIKALTRRHYTGWGKLSAKLI |
| | NGICDKQTGNTILDYLIDDDKINRNFMQLINDDGLSFKEIIQKAQVVGKTNDVKQVVQELPGSPAIK |
| | KGILQSIKLVDELVKVMGHEPEQIVIEMARENQTTNQGKRNSQQRYKRIEDGVKNLASDLNGNILK |
| | EYPTDNQALQNDRLFLYYLQNGKDMYTGEPLDINQLSNYDIDHIVPQAFIKDDSLDNRVLTSSKDN |
| | RGKSDNVPSIEVVQKRKAFWQQLLDSKLISERKENNLTKAERGGLDERDKVGFIRRQLVETRQITK |
| | HVAQILDARFNTEVDDTGKRIRKVKIITLKSNLVSNFRKEFGLYKVREINDYHHAHDAYLNAVVAK |
| | AILKKYPKLEPEFVYGDYQKYDVKRYISRSKDPKDIEKATEKYFFYSNLLNFFKEEVHYADGTIIKR |
| | ENIEYSKDTGEIAWNKEKDFATVRKVLSCPQVNIVKKTEVQTGGFSKESILPKRNSDKLIARKKDW |
| | DPKKYGGFDSPTVAYSVLVVAKVEKGKSKKLKSVKELLGITIMERSSFEKNPIDFLEAKGYKEVRK |
| | DLIVKLPKYSLFELENGRKRMLASAGELQKGNELALPSKYVNFLYLASHYEKLKGSPEDNEQKQLF |
| | VEQHKHYLDEIIEQISEFSKRVILADANLDKVLSAYNKHRDKPIREQAENIIHLFTLTNLGAPAAFKY |
| | FDTTIDRKRYTSTKEVLDATFIHQSITGLYETRIDLSQLGGD |
| 113 | MTKPYSIGLDIGTNSVGWAVITDSYKVPSKKMKVLGNTSKKYVKKNLLGALLFESGITAEGRRLK |
| | RTARRRYTRRRNRILYLQEIFSTEMAKIDESFFQRLDDSFLVPDDKRDSKYPIFGNLVKEKTYHDEF |
| | PTIYHLRKYLADSSKKADLRLVYLALAHMIKYRGHFLIEGDFNSRNNDIKKNFQNFLDSYNAIFESD |
| | LSLENSKQLEEIVKDKISKSAKRERVLKLFPDEKSTGLFSEFLKLIVGNQADFKKHFDLEEKAPLQFS |
| | KDTYDEDLENLLGQIGDDFADLFLVAKKLYDAILLSGILTVTDPSTKAPLSASMIERYENHQKDLSA |
| | LKQFIRSNLSEKYDEVFSDQSKDGYAGYINGKTTQEAFYKYIKNLLSKFEGADYFLDKIEREDFLRK |
| | QRTFDNGSIPHQIHLQEMNAIIRRQGEHYPFLKENKEKIEKILTFRIPYYVGPLARGNRDFAWLTRNS |
| | DQAIRPWNFEEVVDKASSAEAFINKMTNFDLYLPEEKVLPKHSLLYETFAVYNELTKVKFIAEGLR |
| | DYQFLDSGQKKQIVNQLFKEKRKVTEKDIIQFLHNVDGYDGIELKGIEKQFNASLATYHDLLKIIKD |
| | KEFMDDPKNEEILENIIHTLTIFEDREMIKQRLAQYDSLFDEKVIKALTRRHYTGWGKLSAKLINGIC |
| | DKQTGKTILDYLIDDGFANRNFMQLINDDTLPFKQIIQKAQIVGDVDDIEQVVHNLPGSPAIKKGIL |
| | QSIKIVDELVKVMGHNPDNIVIEMARENQTTNRGRSQSQQRLKKLQESLKELGSNILNEEKPSYVES |
| | KVENSQLQNDRLFLYYIQNGKDMYTGEELDIDRLSDYDIDHIIPQAFIKDDSIDNRVLTSSAKNRGK |
| | SDDVPSLEVVKKRKTYWHQLLKAKLISQRKFDNLTKAERGGLTEKDKAGFIKRQLVETRQITKHV |
| | AQILDSRFNTKRDENDKVIRDVKVITLKSNLVSQFRKDFGFYKVREINDYHHAHDAYLNAVVGTA |
| | LLKKYPKLAPEFVYGEYKKYDVRKIVRSKDESSLGKATAKMFFYSNLMNFFKKEVRLSDGTVIVR |
| | PVIEYSKDTGEIAWNKEKDFATVRKVLSCPQVNIVKKTEVQTGGFSKESILPKRNSDKLIARKKDW |
| | DPKKYGGFDSPTVAYSVLVVAKVEKGKSKKLKSVKELLGITIMERSSFEKNPIDFLEAKGYKEVRK |
| | DLIVKLPKYSLFELENGRKRMLASAGELQKGNELALPSKYVNFLYLASHYEKLKGSPEDNEQKQLF |
| | VEQHKHYLDEIIEQISEFSKRVILADANLDKVLSAYNKHRDKPIREQAENIIHLFTLTNLGAPAAFKY |
| | FDTTIDRKRYTSTKEVLDATFIHQSITGLYETRIDLSQLGGD |
| 114 | MKKPYSIGLDIGTNSVGWAVITDDYKVPSKKMKVLGNTSKKYVKKNLLGALLFESGITAEGRRLK |
| | RTARRRYTRRRNRILYLQEIFETGMKEIDESFFQRLDDSFLVPDDKRDSKYPIFGNLAQEKTYHDEF |
| | PTIYHLRKYLADSSKKADLRLVYLALAHMIKYRGHFLIEGDFNSRNNDIKKNFQNFLDSYNAIFESD |
| | LSLENSKQLEEIVKDKISKSAKRERVLKLFPDEKSTGLFSEFLKLIVGNQADFKKHFDLEEKAPLQFS |
| | KDTYDEDLENLLGQIGDDFADLFLVAKKLYDAILLSGILTVTDPSTKAPLSASMIERYENHQKDLA |
| | ALKQFIQNNLQEKYDEVFSDQSKDGYAGYIDGKTTQEAFYKYIKNTLSKIEGSDYFLDKIEREDFLR |
| | KQRTFDNGSIPHQIHLQEMNAIIRRQGEHYPFLQENKEKIEKILTFRIPYYVGPLARGNRDFAWLTR |
| | NSDQAIRPWNFEEVVDQASSAEDFINKMTNYDLYLPEEKVLPKHSLLYETFAVYNELTKVKFIAEG |
| | LRDYQFLDSGQKKQIVNQLFKEKRKVTEKDIIHYLHNVDGYDGIELKGIEKQFNASLSTYHDLLKII |
| | RDKDFMDDPKNEEILENIVHTLTIFEDREMIKQRLAQYDSIFDEKVIKALARRHYTGWGRLSAKLIN |
| | GIRDKQSKKTILDYLIDDDKINRNFMQLINDDGLSFKEIIQKAQVVGKTDDVKQVVQELPGSPAIKK |
| | GILQSIKIVDELVKVMGYDPEQIVIEMARENQTTNQGRRNSQQRYKLIEDGVKNLASDLNGNILKE |
| | YPTDNQALQNDRLFLYYLQNGKDMYTGEPLDINQLSSYDIDHIIPQAFIKDDSLDNRVLTSSKDNR |
| | GKSDNVPSIEVVQKRKAFWQQLLDSKLISERKENNLTKAERGGLDERDKVGFIKRQLVETRQITKH |
| | VAQILDARFNTEVNEKKQKIRSVKIITLKSNLVSNFRKEFGLYKVREINDYHHAHDAYLNAVVAKA |
| | ILKKYPKLEPEFVYGDYQKYDIKRYISRSKDPKDIEKATEKYFFYSNLLNFFKEEVHYADGTIVKRE |
| | NIEYSKDTGEIAWNKEKDFATVRKVLSCPQVNIVKKTEVQTGGFSKESILPKRNSDKLIARKKDWD |
| | PKKYGGFDSPTVAYSVLVVAKVEKGKSKKLKSVKELLGITIMERSSFEKNPIDFLEAKGYKEVRKD |
| | LIVKLPKYSLFELENGRKRMLASAGELQKGNELALPSKYVNFLYLASHYEKLKGSPEDNEQKQLFV |
| | EQHKHYLDEIIEQISEFSKRVILADANLDKVLSAYNKHRDKPIREQAENIIHLFTLTNLGAPAAFKYF |
| | DTTIDRKRYTSTKEVLDATFIHQSITGLYETRIDLSQLGGD |
| 115 | MTKPYSIGLDIGTNSVGWAVISDSYKVPSKKMKVLGNTSKKYVKKNLLGALLFESGITAEGRRLKR |
| | TARRRYTRRRNRILYLQEIFSTEMAKIDESFFQRLDDSFLVPDDKRDSKYPIFGNLVEEKVYHDEFPT |
| | IYHLRKYLADSQKKADLRLVYLALAHMIKYRGHFLIEGDFNSRNNDIKKNFQNFLDSYNAIFESDL |
| | SLENSKQLEEIVKDKISKSAKRERVLSLFPDEKSTGLFSEFLKLIVGNQADFKKHFELEEKAPLQFSK |
| | DTYDEDLENLLGQIGDDFADLFLVAKKLYDAILLSGILTVTDPSTKAPLSASMIERYENHQKDLAQL |
| | KQFVKTNLPEKYDEVFSDQSKDGYAGYIDGKTTQEAFYKYIKETLNKVEGADYFLDKIERENFLRK |
| | QRTFDNGSIPHQIHLQEMNAILHRQGEHYPFLKENKEKIEKILTFRIPYYVGPLARGNRDFAWLTRN |
| | SDQAIRPWNFEEVVDQASSAEEFINKMTNYDLYLPEEKVLPKHSLLYETFAVYNELTKVKFIAEGL |
| | RDYQFLDSGQKKQIVNQLFKEKRKVTEKDIIQFLHNVDGYDGIELKGIEKQFNASLSTYHDLLKIIK |
| | DKEFMDDPKNEEILENIIHTLTIFEDREMIKQRLAQYDTLFDEKVIKALTRRHYTGWGKLSAKLINGI |
| | CDKQTGDTILDFLIDDDKINRNFMQLINDDGLSFKDIIQKAQVVGKTDDVKQVVQELPGSPAIKKGI |
| | LQSIKIVDELVKVMGHAPESIVIEMARENQTTARGKKNSQQRYKRIEDALKNLAPGLDSNILKENPT |
| | DNIQLQNDRLFLYYLQNGKDMYTGEPLDINQLSSYDIDHIVPQAFIKDDSLDNRVLTSSKDNRGKS |
| | DNVPSIEVVQKRKAFWQQLLDSKLISERKENNLTKAERGGLDERDKVGFIRRQLVETRQITKHVAQ |
| | ILDARFNTEVDSKGKKIRKVKIITLKSNLVSNFRKEFGLYKVREINDYHHAHDAYLNAVVAKAILK |
| | KYPKLEPEFVYGDYQKYDVKRYISRSKDPKEVEKATQKYFFYSNLLNFFKEEVHYADGTIIKRENIE |
| | YSKDTGEIAWNKEKDFATVRKVLSCPQVNIVKKTEVQTGGFSKESILPKRNSDKLIARKKDWDPK |
| | KYGGFDSPTVAYSVLVVAKVEKGKSKKLKSVKELLGITIMERSSFEKNPIDFLEAKGYKEVRKDLI |
| | VKLPKYSLFELENGRKRMLASAGELQKGNELALPSKYVNFLYLASHYEKLKGSPEDNEQKQLFVE |
| | QHKHYLDEIIEQISEFSKRVILADANLDKVLSAYNKHRDKPIREQAENIIHLFTLTNLGAPAAFKYFD |
| | TTIDRKRYTSTKEVLDATFIHQSITGLYETRIDLSQLGGD |
| 116 | MTKPYSIGLDIGTNSVGWAVITDSYKVPSKKMKVLGNTSKKYVKKNLLGALLFESGITAEGRRLK |
| | RTARRRYTRRRNRILYLQEIFSTEMAKIDESFFQRLDDSFLVPDDKRDSKYPIFGNLVKEKTYHDEF |
| | PTIYHLRKYLADSTKKADLRLVYLALAHMIKYRGHFLIEGDFNSRNNDIKKNFQDFLDSYNAIFESD |
| | LSLENSKQLEEIVKDKISKSAKKDRLLKLFPGEKNSGIFSEFLKLIVGNQADFKKYFNLDEKAPLHES |
| | KESYDEDLETLLGHIGDDYSDVFLKAKKLYDAILLSGILTVTDNETEAPLSSAMIMRYKEHEEDLAL |
| | LKAYIRKISLETYNEVFKDDTKNGYAGYIDGKTSQEDFYVHLKKLLTGLEGADYFLEKINREDLLR |
| | KQRTFDNGSIPYQIHLQEMRAILDKQAKFYPFLAKNKERIEKILTFRIPYYVGPLARGNSDFAWSIRK |
| | RNEKITPWNFEDVIDKESSAEAFINRMTSFDLYLPEEKVLPKHSLLYETFTVYNELTKVRFIAESMR |
| | DYQFLDSKQKKDIVRLYFKGKRKVTDKDIIDYLHAIDGYDGIELKGVEDQFNASLSTYHDLLNIIND |
| | KEFLDDSSNEAIIEEIIHTLTIFEDREMIKQRLSKYEDIFGKSVLKKLSRRHYTGWGKLSAKLINGIRD |
| | EKSGKTILDYLIDDGISNRNFMQLIHDDTLSFKKKIQKAQIIGDQDNIKQVVQSLPGSPAIKKGILQSI |
| | KIVDELVKVMGRRPESIVVEMARENQYTNQGKSNSQQRLKRLEESLKELGSKILKENVPAKLSKID |
| | NNALQNERLYLYYLQNGKDMYTGDDLDIDRLSNYDIDHIIPQAFLKDNSIDNKVLVSSASNRGKSD |
| | DVPSLDVVKKRKTFWHQLLKSQLISQRKFDNLTKAERGGLSPEDKAGFIQRQLVETRQITKHVARL |
| | LDERFNNEKDEDNKALRTVKIITLKSSLVSQFRKDFELYKVREINDFHHAHDAYLNAVVATALLKK |
| | YPKLEPEFVYGDYPKYNSYKSRKSATEKVLFYSNIMNFFKRLVRLSDGRVLIRPVIEYSKDTGEVA |
| | WNKEKDFATVRKVLSCPQVNIVKKTEVQTGGFSKESILPKRNSDKLIARKKDWDPKKYGGFDSPT |
| | VAYSVLVVAKVEKGKSKKLKSVKELLGITIMERSSFEKNPIDFLEAKGYKEVRKDLIVKLPKYSLFE |
| | LENGRKRMLASAGELQKGNELALPSKYVNFLYLASHYEKLKGSPEDNEQKQLFVEQHKHYLDEII |
| | EQISEFSKRVILADANLDKVLSAYNKHRDKPIREQAENIIHLFTLTNLGAPAAFKYFDTTIDRKRYTS |
| | TKEVLDATFIHQSITGLYETRIDLSQLGGD |
| 117 | MTKPYSIGLDIGTNSVGWAVITDSYKVPSKKMKVLGNTSKKYVKKNLLGALLFESGITAEGRRLK |
| | RTARRRYTRRRNRILYLQEIFSTEMAKIDESFFQRLDDSFLVPDDKRDSKYPIFGNLVKEKAYHDEF |
| | PTIYHLRKYLADSSKKADLRLVYLALAHMIKYRGHFLIEGDFNSRNNDIKKQFQDFLDSYNAIFESS |
| | LSLEQSEQVEEIVKDKISKSAKRERVLKLFPDEKSTGLFSEFLKLIVGNQADFKKHFDLEEKAPLQFS |
| | KDTYDEDLENLLGQIGDGFADLFLVAKKLYDAILLSGILTVTDPSTKAPLSASMIERYENHQKDLA |
| | ALKQFIQNNLQEKYDEVFSDQSKDGYAGYIDGKTTQEAFYKYIKNLLSKFEGADYFLEKIEREDFL |
| | RKQRTFDNGSIPHQIHLQEMNAIIRRQGEHYPFLKENKEKIEKILTFRIPYYVGPLARGNRDFAWLTR |
| | NSDQAIRPWNFEEIVDQASSAEDFINKMTNYDLYLPEEKVLPKHSLLYETFAVYNELTKVKFIAEGL |
| | RDYQFLDSGQKKQIVNQLFKEKRKVTEKDIIHYLHNVDGYDGIELKGIEKQFNASLSTYHDLLKIIK |
| | DKEFMDDPKNEEILENIVHTLTIFEDREMIKQRLAQYDSLFDEKVIKALTRRHYTGWGKLSAKLING |
| | ICDKKTGNTILDYLIDDDKINRNFMQLINDDSLSFKDIIQKAQVVGKTDDVKQVVQELPGSPAIKKG |
| | ILQSIKIVDELVKVMGHAPESIVIEMARENQTTNQGRRNSQQRYKRIEEAIKNLSVDLDSKILKEYPT |
| | DNQALQNDRLFLYYLQNGKDMYTGEPLDINQLSNYDIDHIVPQAFIKDDSLDNRVLTSSKDNRGKS |
| | DNVPSLEIVQKRKAFWQQLLDSKLISERKENNLTKAERGGLDERDKVGFIRRQLVETRQITKHVAQ |
| | ILDARFNTEVNDKGQKIRKVKIITLKSNLVSNFRKEFGLYKVREINDYHHAHDAYLNAVVAKAILK |
| | KYPKLEPEFVYGDYQKYDIKRYISRSKDPKDIEKATEKYFFYSNLLNFFKEEVHYADGTIIKRENVE |
| | YSNDTGEIAWNKEKDFATVRKVLSCPQVNIVKKTEVQTGGFSKESILPKRNSDKLIARKKDWDPK |
| | KYGGFDSPTVAYSVLVVAKVEKGKSKKLKSVKELLGITIMERSSFEKNPIDFLEAKGYKEVRKDLI |
| | VKLPKYSLFELENGRKRMLASAGELQKGNELALPSKYVNFLYLASHYEKLKGSPEDNEQKQLFVE |
| | QHKHYLDEIIEQISEFSKRVILADANLDKVLSAYNKHRDKPIREQAENIIHLFTLTNLGAPAAFKYFD |
| | TTIDRKRYTSTKEVLDATFIHQSITGLYETRIDLSQLGGD |
| 118 | MNKPYSIGLDIGTNSVGWAVITDSYKVPSKKMKVLGNTSKKYVKKNLLGALLFESGITAEGRRLK |
| | RTARRRYTRRRNRILYLQEIFGTEMNEIDESFFQRLDDSFLVPDDKRDSKYPIFGNLVEEKTYHDEFP |
| | TIYHLRKYLADSSKKADLRLVYLALAHMIKYRGHFLIEGDFNSRNNDIKKNFQEFLDSYNAIFESDL |
| | SLENSKQLEEIVKDKISKSAKRERVLKLFPDEKSTGLFSEFLKLIVGNQAEFKKHFDLEEKAPLQFSK |
| | DTYDEDLENLLGQIGDDFADLFLVAKKLYDAILLSGILTVTDPSTKAPLSASMIERYENHQKDLAAL |
| | KQFVKNNLPEKYDEVFSDQSKDGYAGYIDGKTTQEAFYKYIKNLLSKFEGADYFLEKIEREDFLRK |
| | QRTFDNGSIPHQIHLQEMQAILHRQGEYYPFLKENREKIEKILTFRIPYYVGPLARGNRDFAWLTRN |
| | SDQAIRPWNFEEVVDQASSAEEFINKMTNYDLYLPEEKVLPKHSLLYETFAVYNELTKVKFIAEGL |
| | RDYQFLDSGQKKQIVNQLFKEKRKVTEKDIIHYLHNVDGYDGIELKGIEKQFNASLSTYHDLLKIIK |
| | DKEFMDDAKNEEILENIVHTLTIFEDREMIKQRLAQYDSLFDEKVIKALTRRHYTGWGKLSAKLIN |
| | GICDKQTGDTILDYLIDDDKINRNFMQLINDDGLSFKEIIQKAQVFGKTDDVKQVVQELPGSPAIKK |
| | GILQSIKIVDELVKVMGHAPESIVIEMARENQTTNQGKRNSQQRYKRIEDGVKNLASDLNGNILKE |
| | YPTNNQALQNDRLFLYYLQNGKDMYTGESLDINQLSSYDIDHIIPQAFIKDDSIDNRVLTSSKDNRG |
| | KSDNVPSLEVVQKRKAFWQQLLDSKLISERKFNNLTKAERGGLDERDKVGFIKRQLVETRQITKHV |
| | AQILDARFNTEVDENNQKIRKVKIITLKSNLVSNFRKEFGLYKVREINDYHHAHDAYLNAVVAKAI |
| | LKKYPKLEPEFVYGDYQKYDLKRYISKSKDPKDIEKATEKFFFYSNLLNFFKEKVTYADGTIKKRE |
| | NIEYSNDTGEIAWNKEKDFATVRKVLSCPQVNIVKKTEVQTGGFSKESILPKRNSDKLIARKKDWD |
| | PKKYGGFDSPTVAYSVLVVAKVEKGKSKKLKSVKELLGITIMERSSFEKNPIDFLEAKGYKEVRKD |
| | LIVKLPKYSLFELENGRKRMLASAGELQKGNELALPSKYVNFLYLASHYEKLKGSPEDNEQKQLFV |
| | EQHKHYLDEIIEQISEFSKRVILADANLDKVLSAYNKHRDKPIREQAENIIHLFTLTNLGAPAAFKYF |
| | DTTIDRKRYTSTKEVLDATFIHQSITGLYETRIDLSQLGGD |
| 119 | MTKPYSIGLDIGTNSVGWAVITDDYKVPSKKMKVLGNTSKKYVKKNLLGALLFESGITAEGRRLK |
| | RTARRRYTRRRNRILYLQEIFSTEMNKIDESFFQRLDDSFLVPDDKRDSKYPIFGNLAQEKTYHDEF |
| | PTIYHLRKYLADSSKKADLRLVYLALAHMIKYRGHFLIEGDFNSRNNDIKKNFQEFLDSYNAIFESD |
| | LSLENSKQLEEIVKDKISKSAKRERVLKLFPDEKSTGLFSEFLKLIVGNQADFKKHFDLEEKAPLQFS |
| | KDTYDEDLENLLGQIGDGFADLFLVAKKLYDAILLSGILTVTDPSTKAPLSASMIERYENHQKDLA |
| | ALKQFVRTNLPEKYDEVFSDQSKDGYAGYIDGKTTQEAFYKYLKKLLTGLEGADYFLEKIEREDFL |
| | RKQRTFDNGTIPYQLHLEELKAIIHNQGEYYPFLQENKEKIEKILTFRIPYYVGPLARGNRDFAWLT |
| | RNSDQAIRPWNFEEVVDQASSAEDFINKMTNYDLYLPEEKVLPKHSLLYETFAVYNELTKVKFIAE |
| | GLRDYQFLDSGQKKQIVNQLFKEKRKVTEKDIIHYLHNVDGYDGIELKGIEKQFNASLSTYHDLLKI |
| | IKDKEFMDDPKNEEILENIVHTLTIFEDREMIKQRLAQYDSLFDEKVIKALTRRHYTGWGKLSAKLI |
| | NGICDKQTGDTILDYLIDDDKINRNFMQLINDDGLSFKEIIQKAQVVGKTDDVKQVVQELPGSPAIK |
| | KGILQSIKIVDELVKVMGHAPESIVIEMARENQTTARGKKNSQQRYKRIEDALKNLAPGLDSNILKE |
| | HPTDNIQLQNDRLFLYYLQNGKDMYTGEPLDINQLSSYDIDHIVPQAFIKDDSLDNRVLTSSKDNR |
| | GKSDNVPSLEVVQKRKAFWQQLLDSKLISERKENNLTKAERGGLDERDKVGFIRRQLVETRQITKH |
| | VAQILDARFNTEVNEKGKKIRKVKIITLKSNLVSNFRKEFGLYKVREINDYHHAHDAYLNAVVAK |
| | AILKKYPKLEPEFVYGDYQKYDLKRYISKSKDPKEVEKATQKYFFYSNLLNFFKEEVHYADGTIIK |
| | RENIEYSKDTGEIAWNKEKDFATVRKVLSCPQVNIVKKTEVQTGGFSKESILPKRNSDKLIARKKD |
| | WDPKKYGGFDSPTVAYSVLVVAKVEKGKSKKLKSVKELLGITIMERSSFEKNPIDFLEAKGYKEVR |
| | KDLIVKLPKYSLFELENGRKRMLASAGELQKGNELALPSKYVNFLYLASHYEKLKGSPEDNEQKQ |
| | LFVEQHKHYLDEIIEQISEFSKRVILADANLDKVLSAYNKHRDKPIREQAENIIHLFTLTNLGAPAAF |
| | KYFDTTIDRKRYTSTKEVLDATFIHQSITGLYETRIDLSQLGGD |
| 120 | MTKPYSIGLDIGTNSVGWAVITDSYKVPSKKMKVLGNTSKKYVKKNLLGALLFESGITAEGRRLK |
| | RTARRRYTRRRNRILYLQEIFSTEMAKVDESFFQRLDDSFLVPDDKRDSKYPIFGNLVQEKTYHDEF |
| | PTIYHLRKYLADSSKKADLRLVYLALAHMIKYRGHFLIEGDFNSKNNDIKKNFQEFLDSYNAIFESD |
| | LSLENNAQVEEIVKDKISKSAKRERVLKLFPDEKSTGLFSEFLKLIVGNQADFKKHFDLEEKAPLQF |
| | SKDTYDEDLENLLGQIGDGFADLFLVAKKLYDAILLSGILTVTDPSTKAPLSASMIERYENHQKDLA |
| | ALKQFVKANLPEKYDEVFSDQSKDGYAGYIDGKTTQEAFYKYIKNLLAKFEGADYFLEKIEREDFL |
| | RKQRTFDNGVIPYQVHLTEMRAILHRQGEYYPFLKENREKIEKILTFRIPYYVGPLARGNRDFAWLT |
| | RNSDQAIRPWNFEEIVDKASSAEDFINKMTNYDLYLPEEKVLPKHSLLYETFAVYNELTKVKFIAEG |
| | LRDYQFLDSGQKKQIVNQLFKEKRKVTEKDIIHYLHNVDGYDGIELKGIEKQFNASLSTYHDLLKII |
| | KDKDFMDDPKNEEILENIVHTLTIFEDREMIKQRLAQYDSLFDEKVIKALTRRHYTGWGKLSAKLI |
| | NGICDKQTGDTILDYLIDDDKINRNFMQLINDDGLSFKEIIQKAQVVGKTDDVKQVVQELPGSPAIK |
| | KGILQSIKIVDELVKVMGHAPESIVIEMARENQTTNQGKRNSQQRYKKIEDAIKNLDSNLNSKILKE |
| | YPTNNQALQNDRLFLYYLQNGKDMYTGEELDINQLSNYDIDHIIPQAFIKDDSLDNRVLTSSKDNR |
| | GKSDNVPSIEVVQKRKAFWQQLLDSKLISERKENNLTKAERGGLDERDKVGFIKRQLVETRQITKH |
| | VAQILDARFNTEVDENGKRIRKVKIITLKSNLVSNFRKEFGLYKVREINDYHHAHDAYLNAVVAKA |
| | ILKKYPKLEPEFVYGDYQKYDIKRYISRSKDPKEVEKATEKYFFYSNLLNFFKEEVTYADGTIKKRE |
| | NIEYSNDTGEIAWNKEKDFATVRKVLSCPQVNIVKKTEVQTGGFSKESILPKRNSDKLIARKKDWD |
| | PKKYGGFDSPTVAYSVLVVAKVEKGKSKKLKSVKELLGITIMERSSFEKNPIDFLEAKGYKEVRKD |
| | LIVKLPKYSLFELENGRKRMLASAGELQKGNELALPSKYVNFLYLASHYEKLKGSPEDNEQKQLFV |
| | EQHKHYLDEIIEQISEFSKRVILADANLDKVLSAYNKHRDKPIREQAENIIHLFTLTNLGAPAAFKYF |
| | DTTIDRKRYTSTKEVLDATFIHQSITGLYETRIDLSQLGGD |
| 121 | MTKPYSIGLDIGTNSVGWAVITDNYKVPSKKMKVLGNTSKKYVKKNLLGALLFESGITAEGRRLK |
| | RTARRRYTRRRNRILYLQEIFSTEMSKIDESFFQRLDDSFLVPDDKRDSKYPIFGNLVEEKTYHDEFP |
| | TIYHLRKYLADSSKKADLRLVYLALAHMIKYRGHFLIEGDFNSRNNDIKKNFQNFLDSYNAIFESDL |
| | SLENSEQLEEIVKDKISKSAKRERVLKLFPDEKSTGLFSEFLKLIVGNQAEFKKHFDLEEKAPLQFSK |
| | DTYDEDLENLLGQIGDDFADLFLVAKKLYDAILLSGILTVTDPSTKAPLSASMIERYENHQKDLAAL |
| | KQFIQNNLQEKYDEVFSDQSKDGYAGYIDGKTTQEAFYKYIKNLLSKLEGADYFLDKIEREDFLRK |
| | QRTFDNGSIPHQIHLQEMNAILRRQGEHYPFLKENKEKIEKILTFRIPYYVGPLARGNRDFAWLTRN |
| | SDQAIRPWNFEEIVDQASSAEDFINKMTNYDLYLPEEKVLPKHSLLYETFAVYNELTKVKFIAEGLR |
| | DYQFLDSGQKKQIVNQLFKDKRKVTEKDIIHYLHNVDGYDGIELKGIEKQFNASLSTYHDLLKIIKD |
| | KEFMDDPKNEEILENIVHTLTIFEDREMIKQRLAQYDSLFDEKVIKALTRRHYTGWGKLSAKLINGI |
| | CDKQTGDTILDYLIDDDKINRNFMQLINDDGLSFKEIIQKAQVVGKTDDVKQVVQELPGSPAIKKGI |
| | LQSIKIVDELVKVMGHAPESIVIEMARENQTTNQGKRNSQQRYKRIEDGIKNLASDLNGNILKEYPT |
| | DNQALQNDRLFLYYLQNGKDMYTGEPLDINQLSNYDIDHIVPQAFIKDDSLDNRVLTSSKDNRGKS |
| | DNVPSLEVVQKRKAFWQQLLDSQLISQRKFDNLTKAERGGLTDHDKAGFIKRQLVETRQITKHVA |
| | QILDARFNTEVTEKDKKIRNVKIITLKSNLVSNFRKEFGLYKVREINDYHHAHDAYLNAVVAKAIL |
| | KKYPKLEPEFVYGDYQKYDIKRYISRSKDPKDIDKATEKYFFYSNLLNFFKEEVHYADGTIVKRENI |
| | EYSKDTGEIAWNKEKDFATVRKVLSCPQVNIVKKTEVQTGGFSKESILPKRNSDKLIARKKDWDPK |
| | KYGGFDSPTVAYSVLVVAKVEKGKSKKLKSVKELLGITIMERSSFEKNPIDFLEAKGYKEVRKDLI |
| | VKLPKYSLFELENGRKRMLASAGELQKGNELALPSKYVNFLYLASHYEKLKGSPEDNEQKQLFVE |
| | QHKHYLDEIIEQISEFSKRVILADANLDKVLSAYNKHRDKPIREQAENIIHLFTLTNLGAPAAFKYFD |
| | TTIDRKRYTSTKEVLDATFIHQSITGLYETRIDLSQLGGD |
| 122 | MTKPYSIGLDIGTNSVGWAVITDSYKVPSKKMKVLGNTSKKYVKKNLLGALLFESGITAEGRRLK |
| | RTARRRYTRRRNRILYLQEIFSTEMAKIDESFFQRLDDSFLVPDDKRDSKYPIFGNLVKEKTYHDEF |
| | PTIYHLRKYLADSSKKADLRLVYLALAHMIKYRGHFLIEGDFNSRNNDIKKNFQNFLDSYNAIFESD |
| | LSLENSKQLEEIVKDKISKSAKRERVLKLFPDEKSTGLFSEFLKLIVGNQADFKKHFDLEEKAPLQFS |
| | KDTYDEDLENLLGQIGDDFADLFLVAKKLYDAILLSGILTVTDPSTKAPLSASMIERYENHQKDLA |
| | ALKQFIKSNLSEKYDEVFSDQSKDGYAGYIDGKTTQEAFYKYIKNLLSKLEGSDYFLDKIEREDFLR |
| | KQRTFDNGSIPHQIHLQEMNAIIRRQGEHYPFLKENKEKIEKILTFRIPYYVGPLARGNRDFAWLTR |
| | NSDQAIRPWNFEEVVDKASSAEDFINKMTNYDLYLPEEKVLPKHSLLYETFAVYNELTKVKFIAEG |
| | LRDYQFLDSGQKKQIVNQLFKEKRKVTEKDIIQYLHNVDGYDGIELKGIEKQFNASLSTYHDLLKII |
| | KDKDFMDDPKNEEILENIVHTLTIFEDREMIKQRLAQYDSLFDEKVIKALTRRHYTGWGKLSAKLI |
| | DGIYDKQSRKTILDYLIDDGEINRNFMQLIHDDGLSFKEIIQKAQVVGETDNLKQVVQELPGSPAIK |
| | KGILQSIKIVDELVKVMGHAPESIVIEMARENQTTARGKKNSQQRYKRIEDALKNLAPGLDSNILKE |
| | NPTDNVQLQNDRLFLYYLQNGKDMYTGEPLDINQLSSYDIDHIVPQAFIKDDSLDNRVLTSSKDNR |
| | GKSDNVPSLEVVQKRKAFWQQLLDSKLISQRKFDNLTKAERGGLTENDKAGFIKRQLVETRQITK |
| | HVAQILDARFNTEVNENNQKIRKVKIITLKSNLVSNFRKEFGLYKVREINDYHHAHDAYLNAVVA |
| | KAILKKYPKLEPEFVYGDYQKYDIKRYISRSKDPKDIEKATEKYFFYSNLLNFFKEEVHYADGTIIK |
| | RENIEYSNDTGEIAWNKEKDFATVRKVLSCPQVNIVKKTEVQTGGFSKESILPKRNSDKLIARKKD |
| | WDPKKYGGFDSPTVAYSVLVVAKVEKGKSKKLKSVKELLGITIMERSSFEKNPIDFLEAKGYKEVR |
| | KDLIVKLPKYSLFELENGRKRMLASAGELQKGNELALPSKYVNFLYLASHYEKLKGSPEDNEQKQ |
| | LFVEQHKHYLDEIIEQISEFSKRVILADANLDKVLSAYNKHRDKPIREQAENIIHLFTLTNLGAPAAF |
| | KYFDTTIDRKRYTSTKEVLDATFIHQSITGLYETRIDLSQLGGD |
| 123 | MTKPYSIGLDIGTNSVGWAVITDSYKVPSKKMKVLGNTSKKYVKKNLLGALLFESGITAEGRRLK |
| | RTARRRYTRRRNRILYLQEIFSTEMAEIDESFFQRLDDSFLVPDDKRDSKYPIFGNLVKEKTYHDEFP |
| | TIYHLRKYLADSTKKADLRLVYLALAHMIKYRGHFLIEGDFNSRNNDIKKNFQDFLDSYNAIFESD |
| | LSLENSKQLEEIVKDKISKSAKRERVLKLFPDEKSTGLFSEFLKLIVGNQADFKKHFDLEEKAPLQFS |
| | KDTYDEDLENLLGQIGDDFADLFLVAKKLYDAILLSGILTVTDPSTKAPLSASMIERYENHQKDLA |
| | QLKQFVKTNLPEKYDEVFSDQSKDGYAGYINGKTTQEAFYKYIKNLLSKLEGADYFLDKIEREDFL |
| | RKQRTFDNGSIPHQIHLQEMNAILHRQGEHYPFLKENKEKIQQLLTFRIPYYVGPLARGNRDFAWL |
| | TRNSDQAIRPWNFEEIVDKASSAEDFINKMTNYDLYLPEEKVLPKHSLLYETFAVYNELTKVKFIAE |
| | GLRDYQFLDSGQKKQIVNQLFKEKRKVTEKDIIHYLHNVDGYDGIELKGIEKQFNASLSTYHDLLKI |
| | IKDKDFMDDPKNEEILENIVHTLTIFEDREMIKQRLAQYDTLFDEKVIKALARRHYTGWGKLSAKLI |
| | NGIYDKQSKKTILDYLIDDDKINRNFMQLINDDGLSFKDIIQKAQVVGKTDDVKQVVQELPGSPAIK |
| | KGILQSIKLVDELVKVMGHAPESIVIEMARENQTTARGKKNSQQRYKRIEDALKNLAPGLDSNILK |
| | ENPTDNIQLQNDRLFLYYLQNGKDMYTGEALDINQLSNYDIDHIVPQAFIKDDSLDNRVLTSSKDN |
| | RGKSDNVPSLEVVQKRKAFWQQLLDSKLISERKENNLTKAERGGLDERDKVGFIKRQLVETRQITK |
| | HVAQILDARFNTEVNEKKQKIRNVKIITLKSNLVSNFRKEFGLYKVREINDYHHAHDAYLNAVVA |
| | KAILKKYPKLEPEFVYGDYQKYDIKRYISRSKDPKDIEKATQKYFFYSNLLNFFKEEVHYADGTIVK |
| | RENVEYSNDTGEIAWNKEKDFATVRKVLSCPQVNIVKKTEVQTGGFSKESILPKRNSDKLIARKKD |
| | WDPKKYGGFDSPTVAYSVLVVAKVEKGKSKKLKSVKELLGITIMERSSFEKNPIDFLEAKGYKEVR |
| | KDLIVKLPKYSLFELENGRKRMLASAGELQKGNELALPSKYVNFLYLASHYEKLKGSPEDNEQKQ |
| | LFVEQHKHYLDEIIEQISEFSKRVILADANLDKVLSAYNKHRDKPIREQAENIIHLFTLTNLGAPAAF |
| | KYFDTTIDRKRYTSTKEVLDATFIHQSITGLYETRIDLSQLGGD |
| 124 | MKKPYSIGLDIGTNSVGWAVITDDYKVPSKKMKVLGNTSKKYVKKNLLGALLFESGITAEGRRLK |
| | RTARRRYTRRRNRILYLQEIFSTEMSKIDESFFQRLDDSFLVPDDKRDSKYPIFGNLVEEKTYHDEFP |
| | TIYHLRKYLADSSKKADLRLVYLALAHMIKYRGHFLIEGDFNSRNNDIKKNFQNFLDSYNAIFESDL |
| | SLENSKQLEEIVKDKISKSAKRERVLKLFPDEKSTGLFSEFLKLIVGNQADFKKHFDLEEKAPLQFSK |
| | DTYDEDLENLLGQIGDDFADLFLVAKKLYDAILLSGILTVTDPSTKAPLSASMIERYENHQKDLAAL |
| | KQFIQNNLQEKYDEVFSDQSKDGYAGYIDGKTTQEAFYKYIKNLLSKFEGADYFLDKIEREDFLRK |
| | QRTFDNGSIPHQIHLQEMNAIIRRQGEHYPFLKENKEKIEKILTFRIPYYVGPLARGNRDFAWLTRNS |
| | DQAIRPWNFEEVVDKASSAEDFINKMTNYDLYLPEEKVLPKHSLLYETFAVYNELTKVKFIAEGLR |
| | DYQFLDSGQKKQIVNQLFKEKRKVTEKDIIHYLHTVDGYDGIELKGIEKQFNASLSTYHDLLKIIKD |
| | KEFMDDPKNEEILENIIHTLTIFEDREMIKQRLAQYDSLFDEKVIKALTRRHYTGWGKLSAKLINGIR |
| | DDKSGNTILDYLIDDDKINRNFMQLIHDDGLSFKEIIQKAQVFGKTDDVKQVVQELPGSPAIKKGIL |
| | QSIKIVDELVKVMGHAPESIVIEMARENQTTNQGKRNSQQRYKRIEEGIKNLASELNGNILKEYPTN |
| | NQALQNERLFLYYLQNGRDMYTGKSLDINQLSNYDIDHIIPQAFIKDDSLDNRVLTSSKDNRGKSD |
| | NVPSIEVVEKMKAFWQQLLDSKLISERKENNLTKAERERGGLNELDKVGFIKRQLVETRQITKHVA |
| | QILDARFNTEVNEKKQKIREVKIITLKSNLVSNFRKEFGLYKVREINDYHHAHDAYLNAVVAKAIL |
| | KKYPKLEPEFVYGDYQKYDIKRYISRSKDPKEIEKATEKYFFYSNLLNFFKEEVHYADGTIVKRENI |
| | EYSKDTGEIAWNKEKDFATVRKVLSCPQVNIVKKTEVQTGGFSKESILPKRNSDKLIARKKDWDPK |
| | KYGGFDSPTVAYSVLVVAKVEKGKSKKLKSVKELLGITIMERSSFEKNPIDFLEAKGYKEVRKDLI |
| | VKLPKYSLFELENGRKRMLASAGELQKGNELALPSKYVNFLYLASHYEKLKGSPEDNEQKQLFVE |
| | QHKHYLDEIIEQISEFSKRVILADANLDKVLSAYNKHRDKPIREQAENIIHLFTLTNLGAPAAFKYFD |
| | TTIDRKRYTSTKEVLDATFIHQSITGLYETRIDLSQLGGD |
| 125 | MTKPYSIGLDIGTNSVGWAVITDSYKVPSKKMKVLGNTSKKYVKKNLLGALLFESGITAEGRRLK |
| | RTARRRYTRRRNRILYLQEIFSTEMAKVDESFFQRLDDSFLVPDDKRDSKYPIFGNLVKEKTYHDEF |
| | PTIYHLRKYLADSSKKADLRLVYLALAHMIKYRGHFLIEGDFNSRNNDIKKNFQDFLDSYNAIFESD |
| | LSLENSKQLEEIVKDKISKSAKRERVLKLFPDEKSTGLFSEFLKLIVGNQAEFKKHFDLEEKAPLQFS |
| | KDTYDEDLENLLGQIGDDFADLFLVAKKLYDAILLSGILTVTDPSTKAPLSASMIERYENHQKDLAT |
| | LKQFIKNNLSEKYDEVFSDQSKDGYAGYIDGKTTQEAFYKYIKNLLSKLEGADYFLDKIEREDFLR |
| | KQRTFDNGSIPHQIHLQEMNAILRRQGEHYPFLKENKEKIEKILTFRIPYYVGPLARGNRDFAWLTR |
| | NSDQAIRPWNFEEIVDQASSAEDFINKMTNYDLYLPEEKVLPKHSLLYETFAVYNELTKVKFIAEGL |
| | RDYQFLDSGQKKQIVNQLFKEKRKVTEKDIIHYLHNVDGYDGIELKGIEKQFNASLSTYHDLLKIIK |
| | DKEFMDDPKNEEILENIVHTLTIFEDREMIKQRLAQYDSLFDEKVIKALTRRHYTGWGKLSAKLING |
| | IYDKQSKKTILDYLIDDDKINRNFMQLIHDDGLSFKDIIQKAQVVGKTDDVKQVVQELPGSPAIKKG |
| | ILQSIKIVDELVKVMGHAPESIVIEMARENQTTARGKKNSQQRYKRIEDALKNLAPGLDSNILKENP |
| | TDNIQLQNDRLFLYYLQNGKDMYTGEPLDINQLSSYDIDHIVPQAFIKDDSLDNRVLTSSKDNRGK |
| | SDNVPSLEVVQKRKGFWQQLLDSKLISERKENNLTKAERGGLDERDKVGFIKRQLVETRQITKHVA |
| | QILDARFNTEVNEKNQKIRTVKIITLKSNLVSNFRKEFRLYKVREINDYHHAHDAYLNAVVAKAIL |
| | KKYPKLEPEFVYGDYQKYDVRRYLSSKDSKDFGKATAKMFFYSNLLNFFKKEVRLADGTVIVRPV |
| | IEYSKDTGEIAWNKEKDFATVRKVLSCPQVNIVKKTEVQTGGFSKESILPKRNSDKLIARKKDWDP |
| | KKYGGFDSPTVAYSVLVVAKVEKGKSKKLKSVKELLGITIMERSSFEKNPIDFLEAKGYKEVRKDL |
| | IVKLPKYSLFELENGRKRMLASAGELQKGNELALPSKYVNFLYLASHYEKLKGSPEDNEQKQLFVE |
| | QHKHYLDEIIEQISEFSKRVILADANLDKVLSAYNKHRDKPIREQAENIIHLFTLTNLGAPAAFKYFD |
| | TTIDRKRYTSTKEVLDATFIHQSITGLYETRIDLSQLGGD |
| 126 | MKKPYSIGLDIGTNSVGWAVVTDDYKVPAKKMKVLGNTDKSHIKKNLLGALLFESGETAEGRRL |
| | KRTARRRYTRRRNRILYLQEIFATEMNKIDESFFQRLDDSFLVPDDKRDSKYPIFGNLAQEKTYHEK |
| | FPTIYHLRKYLADSSKKADLRLVYLALAHMIKYRGHFLIEGDFNSRNNDIKKNFQNFLDSYNAIFES |
| | DLSLENSKQLEEIVKDKISKSAKRERVLKLFPDEKSTGLFSEFLKLIVGNQADFKKHFDLEEKAPLQF |
| | SKDTYDEDLENLLGQIGDDFADLFLVAKKLYDAILLSGILTVTDPSTKAPLSASMIERYENHQKDLA |
| | TLKQFIKNNLSEKYDEVFSDQSKDGYAGYIDGKTTQEAFYKYIKNLLSKLEGADYFLDKIEREDFL |
| | RKQRTFDNGSIPHQIHLQEMNAIIRRQGEHYPFLQENKEKIEKILTFRIPYYVGPLARGNRDFAWLTR |
| | NSDQAIRPWNFEEIVDQASSAEDFINKMTNYDLYLPEEKVLPKHSLLYETFAVYNELTKVKFIAEGL |
| | RDYQFLDSGQKKQIVNQLFKEKRKVTEKDIIHYLHTVDGYDGIELKGIEKQFNASLSTYHDLLKIIK |
| | DKEFMDDPKNEEILENIIHTLTIFEDREMIKQRLAQYDSIFDEKVIKALTRRHYTGWGKLSAKLINGI |
| | YDKQSKKTILDYLIDDDKINRNFMQLINDDGLSFKEIIQKAQVVGKTDDVKQVVQELPGSPAIKKGI |
| | LQSIKIVDELVKVMGGDPESIVVEMARENQFTNQGRRNSQQRYKKIKEAIKNLDSDLNSKILKEYPT |
| | DNQALQNDRLFLYYLQNGKDMYTGEALDINQLSNYDIDHIIPQAFIKDDSLDNRVLTSSKDNRGKS |
| | DNVPSLEIVEKMKGFWRQLLDSKLISQRKFDNLTKAERGGLTEHDKAGFIKRQLVETRQITKHVAQ |
| | ILDARFNTEVNDKKQKIRNVKIITLKSNLVSNFRKEFGLYKVREINDYHHAHDAYLNAVVAKAILK |
| | KYPKLEPEFVYGDYQKYDIKRYISRSKDPKEIEKATEKYFFYSNLLNFFKEEVHYADGTIIKRENIEY |
| | SKDTGEIAWNKEKDFATVRKVLSCPQVNIVKKTEVQTGGFSKESILPKRNSDKLIARKKDWDPKK |
| | YGGFDSPTVAYSVLVVAKVEKGKSKKLKSVKELLGITIMERSSFEKNPIDFLEAKGYKEVRKDLIV |
| | KLPKYSLFELENGRKRMLASAGELQKGNELALPSKYVNFLYLASHYEKLKGSPEDNEQKQLFVEQ |
| | HKHYLDEIIEQISEFSKRVILADANLDKVLSAYNKHRDKPIREQAENIIHLFTLTNLGAPAAFKYFDT |
| | TIDRKRYTSTKEVLDATFIHQSITGLYETRIDLSQLGGD |
| 127 | MTKPYSIGLDIGTNSVGWAVITDSYKVPSKKMKVLGNTSKKYVKKNLLGALLFESGITAEGRRLK |
| | RTARRRYTRRRNRILYLQEIFSTEMAKIDESFFQRLDDSFLVPDDKRDSKYPIFGNLVEEKAYHDEF |
| | PTIYHLRKYLADSSKKADLRLVYLALAHMIKYRGHFLIEGDFNSRNNDIKKNFQDFLDSYNAIFESD |
| | LSLENSKQLEEIVKDKISKSAKKDRILKLFPGEKNSGIFSEFLKLIVGNQADFKKYFNLDEKAPLHES |
| | KESYDEDLETLLGHIGDDYSDVFLKAKKLYDAILLSGILTVTDNETEAPLSSAMIMRYKEHEEDLAL |
| | LKAYIRNISLETYNEVFKDDTKNGYAGYIDGKTNQEDFYVYLKKLLTGLEGADYFLEKINREDLLR |
| | KQRTFDNGSIPHQIHLQEMHAILRKQEKFYPFLAKNKERIEKILTFRIPYYVGPLARGNSDFAWSIRK |
| | RNEKITPWNFEDVIDKESSAEAFINRMTSFDLYLPEEKVLPKHSLLYETFTVYNELTKVRFIAESMR |
| | DYQFLDSKQKKDIVRLYFKGKRKVTDKDIIDYLHAIDGYDGIELKGIEKQFNSSLSTYHDLLNIIND |
| | KEFLDDSSNEAIIEEIIHTLTIFEDREMIKQRLSKFENIFDKSVLKKLSRRHYTGWGKLSAKLINGIRD |
| | EKSGKTILDYLIDDGVSNRNFMQLIHDDTLSFKKKIQKAQIIGDQDNIKQVVQSLPGSPAIKKGILQSI |
| | KIVDELVKVMGRNPESIVVEMARENQYTNQGKSNSQQRLKRLEESLKELGSKILKENVPAKLSKID |
| | NNALQNERLYLYYLQNGKDMYTGDDLDIDRLSNYDIDHIIPQAFLKDNSIDNKVLVSSASNRGKSD |
| | DVPSLDVVKKRKTFWHQLLKSKLISQRKFDNLTKAERGGLTPEDKAGFIQRQLVETRQITKHVAQL |
| | LDNRFNEKRDEDKKVIRKVNIVLLKSSLVSQFRKDFELYKVREINDFHHAHDAYLNAVVATALLK |
| | KYPKLEPEFVYGEYSKFKVSKDIDKATEKYFFYSNLLNFFKEKVLYADGTVVFRKNVEYSKDTGEI |
| | AWNKEKDFATVRKVLSCPQVNIVKKTEVQTGGFSKESILPKRNSDKLIARKKDWDPKKYGGFDSP |
| | TVAYSVLVVAKVEKGKSKKLKSVKELLGITIMERSSFEKNPIDFLEAKGYKEVRKDLIVKLPKYSLF |
| | ELENGRKRMLASAGELQKGNELALPSKYVNFLYLASHYEKLKGSPEDNEQKQLFVEQHKHYLDEI |
| | IEQISEFSKRVILADANLDKVLSAYNKHRDKPIREQAENIIHLFTLTNLGAPAAFKYFDTTIDRKRYT |
| | STKEVLDATFIHQSITGLYETRIDLSQLGGD |
| 128 | MTKPYSIGLDIGTNSVGWAVITDSYKVPSKKMKVLGNTSKKYVKKNLLGALLFESGITAEGRRLK |
| | RTARRRYTRRRNRILYLQEIFSTEMAKIDESFFQRLDDSFLVPDDKRDSKYPIFGNLVEEKTYHDEFP |
| | TIYHLRKYLADSSKKADLRLVYLALAHMIKYRGHFLIEGDFNSRNNDIKKNFQEFLDSYNAIFESDL |
| | SLENSKQLEEIVKDKISKSAKRERVLKLFPDEKSTGLFSEFLKLIVGNQADFKKHFDLEEKAPLQFSK |
| | DTYDEDLENLLGQIGDDFADLFLVAKKLYDAILLSGILTVTDPSTKAPLSASMIERYENHQKDLAAL |
| | KQFIQNNIQEKYDEVFSDQSKDGYAGYINGKTTQEAFYKYIKNLLSKLEGADYFLDKIEREDFLRK |
| | QRTFDNGSIPHQIHLQEMNAIIRRQGEHYPFLQENKEKIEKILTFRIPYYVGPLARGNRDFAWLTRNS |
| | DQAIRPWNFEEVVDQASSAEEFINKMTNYDLYLPEEKVLPKHSLLYETFAVYNELTKVKFIAEGLR |
| | DYQFLDSGQKKQIVNQLFKEKRKVTEKDIIHYLHNVDGYDGIELKGIEKQFNASLSTYHDLLKIIKD |
| | KEFMDDPKNEEILENIIHTLTIFEDREMIKQRLAQYDSIFDEKVIKALTRRHYTGWGKLSAKLINGIY |
| | DKQSKKTILDYLIDDDKINRNFMQLINDDGLSFKEIIQKAQVVGKTDDVKQVVQELPGSPAIKKGIL |
| | QSIKLVDELVKVMGHAPESIVIEMARENQTTARGKKNSQQRYKRIEDALKNLAPGLDSNILKEHPT |
| | DNIQLQNDRLFLYYLQNGKDMYTGNPLDINQLSSYDIDHIVPQAFIKDDSLDNRVLTSSKDNRGKS |
| | DNVPSLEVVQKRKAFWQQLLDSKLISEHKFNNLTKAERGGLDRQDKVGFIKRQLVETRQITKHVA |
| | QILDARFNTEVNENNQKIRKVKIITLKSNLVSNFRKEFGLYKVREINDYHHAHDAYLNAVVAKAIL |
| | KKYPKLEPEFVYGDYQKYDIKRYISRSKDPKEIEKATEKYFFYSNLLNFFKEEVHYADGTIVKRENI |
| | EYSNDTGEIAWNKEKDFATVRKVLSCPQVNIVKKTEVQTGGFSKESILPKRNSDKLIARKKDWDPK |
| | KYGGFDSPTVAYSVLVVAKVEKGKSKKLKSVKELLGITIMERSSFEKNPIDFLEAKGYKEVRKDLI |
| | VKLPKYSLFELENGRKRMLASAGELQKGNELALPSKYVNFLYLASHYEKLKGSPEDNEQKQLFVE |
| | QHKHYLDEIIEQISEFSKRVILADANLDKVLSAYNKHRDKPIREQAENIIHLFTLTNLGAPAAFKYFD |
| | TTIDRKRYTSTKEVLDATFIHQSITGLYETRIDLSQLGGD |
| 129 | MNKPYSIGLDIGTNSVGWAVITDDYKVPSKKMKVLGNTDKKYIKKNLLGALLFEGGETAEGRRLK |
| | RTARRRYTRRRNRILYLQEIFAAEMNKVDESFFHRLDDSFLVPEDKRGSKYPIFGTLEEEKEYHKKF |
| | PTIYHLRKHLADSKEKADLRLIYLALAHMIKYRGHFLYEDSFDIKNNDIQKIFNEFTSIYDNTFEESS |
| | LSKGNAQVEEIFTDKISKSAKRERVLKLFPDEKSTGLFSEFLKLIVGNQADFKKHFDLEEKAPLQFS |
| | KDTYDEDLENLLGQIGDMYADLFLVAKKLYDAILLSGILTVTDPTTKAPLSASMIERYENHQEDLA |
| | ALKQFIKNNLSEKYAEVFSDASKDGYAGYIEGKTTQENFYKFIKKAIEKIEGSDYFIDKIEREDFLRK |
| | QRTFDNGSIPHQIHLQEMHAIIRRQAEFYPFLAENQDKIEKILTFRIPYYVGPLARGKSEFAWLNRKS |
| | DEKIRPWNFDEMVDKETSAENFITRMTNYDQYLPDQKVLPKHSLLYEKFAVYNELTKVRYVTEQG |
| | KSFFFDANMKQEIFDKTFKVYRKVTKEKLMDFLGKEFDEFRIVDLMGLDKDNKSFNASLGTYHDL |
| | KKIVSKDFLDNPENEDILENVVLTLTLFEDREMIRKRLEKYKDVLTEEQRKKLERRHYTGWGRLSA |
| | KLINGIRDKVTRKTILDYLIDDGTSNRNFMQLIHDDTLSFVDEIRLAQSSGEAEDYRAEVQNLAGSP |
| | AIKKGILQSLKIVDELIEVMGYDPEHIVVEMARENQFTNQGRRNSQQRYKKIKDAIKNLDSNLNSKI |
| | LKEYPTNNQALQNDRLFLYYLQNGKDMYTGEALDINQLSNYDIDHIIPQAFIKDDSLDNRVLTSSK |
| | DNRGKSDNVPSIEVVEKMKAFWQQLLDSKLISERKYNNLTKAERERGGLNELDKVGFIKRQLVET |
| | RQITKHVAQILDAKFNTEKDENGKRIRKVKIITLKSNLVSNFRKEFGLYKVREINDYHHAHDAYLN |
| | AVVAKAILKKYPKLEPEFVYGDYQKYDIKRYISRSKDPKEIEKATEKYFFYSNLLNFFKEEVHYAD |
| | GTIIKRENIEYSKDTGEIAWNKEKDFATVRKVLSCPQVNIVKKTEVQTGGFSKESILPKRNSDKLIAR |
| | KKDWDPKKYGGFDSPTVAYSVLVVAKVEKGKSKKLKSVKELLGITIMERSSFEKNPIDFLEAKGY |
| | KEVRKDLIVKLPKYSLFELENGRKRMLASAGELQKGNELALPSKYVNFLYLASHYEKLKGSPEDN |
| | EQKQLFVEQHKHYLDEIIEQISEFSKRVILADANLDKVLSAYNKHRDKPIREQAENIIHLFTLTNLGA |
| | PAAFKYFDTTIDRKRYTSTKEVLDATFIHQSITGLYETRIDLSQLGGD |
| 130 | MKKSYSIGLDIGTNSVGWAVVDDDYKVPAKKMRVFGDTDRSHIKKNLLGTLLFDSGETAESRRLK |
| | RTARRRYTRRRNRILYLQEIFAEEMNKVDDSFFHRLDESFLTDDDKTFDSHPIFGNKAEEDAYHQK |
| | FPTIYHLRKHLADSTEKADLRLVYLALAHMIKFRGHFLIEGELNAENTDVQKLFAQFVGVYDRTFD |
| | DSHLSEITVDASSILTEKISKSRRLENLIKHYPTEKKNTLFGNLVALALGLQPNFKTNFKLSEDAKLQ |
| | FSKDTYEEDLEELLGKIGDEYADLFTSAKNLYDAILLSGILTVDDTSTKAPLSASMIKRYAEHHEDL |
| | EKLKEFIKANKPELYHDIFKDETKNGYAGYIENGVKQDEFYKYLKTTLSKIDGSQYFLDKIEREDFL |
| | RKQRTFDNGSIPHQIHLQEMHAILRRQGEYYPFLKENQAKIEKILTFRIPYYVGPLARKDSRFAWAN |
| | YHSDEPITPWNFDEVVDKEKSAEKFITRMTLNDLYLPEEKVLPKHSHVYETFTVYNELTKIKYVNE |
| | QGESFFFDANMKQEIFDHVFKENRKVTKDKLLDFLNKEFEEFRIVDLIGLDKENKSFNASLGTYHD |
| | LKKILDKSFLDDKTNEQIIEDIVLTLTLFEDRDMIRQRLQKYSDIFTPQQLKKLERRHYTGWGRLSY |
| | KLINGIRNKENNKTILDFLIDDGHANRNFMQLINDESLSFKTIIQEAQVVGDVDDIEAVVHDLPGSPA |
| | IKKGILQSVKIVDELVKVMGDNPDNIVIEMARENQTTNRGRTQSQQRLKKLQESLKELGSNILNEE |
| | KPSYIEDKVENSHLQNDKLFLYYIQNGKDMYTGDELDIDHLSDYDIDHIIPQAFIKDDSIDNRVLTSS |
| | AKNRGKSDDVPSLDIVHDRKADWIRLYKSGLISKRKFDNLTKAERGGLTEADKAGFIKRQLVETR |
| | QITKHVAQILDARFNTKRDENDKVIRDVKIITLKSNLVSQFRKEFGFYKVREINDYHHAHDAYLNA |
| | VVGTALLKKYPKLAPEFVYGEYIKYDIKGDITKSKIETTRDKATAKYFFYSNLLNFFKTKVRYADG |
| | TVIVRPVIEYSKDTGEIAWNKEKDFATVRKVLSCPQVNIVKKTEVQTGGFSKESILPKRNSDKLIAR |
| | KKDWDPKKYGGFDSPTVAYSVLVVAKVEKGKSKKLKSVKELLGITIMERSSFEKNPIDFLEAKGY |
| | KEVRKDLIVKLPKYSLFELENGRKRMLASAGELQKGNELALPSKYVNFLYLASHYEKLKGSPEDN |
| | EQKQLFVEQHKHYLDEIIEQISEFSKRVILADANLDKVLSAYNKHRDKPIREQAENIIHLFTLTNLGA |
| | PAAFKYFDTTIDRKRYTSTKEVLDATFIHQSITGLYETRIDLSQLGGD |
| 131 | MTKPYSIGLDIGTNSVGWAVITDSYKVPSKKMKVLGNTSKKYVKKNLLGALLFESGITAEGRRLK |
| | RTARRRYTRRRNRILYLQEIFSTEMAKIDESFFQRLDDSFLVPDDKRDSKYPIFGNLVEEKTYHDEFP |
| | TIYHLRKYLADSSKKADLRLVYLALAHMIKYRGHFLIEGDFNSRNNDIKKNFQDFLDSYNAIFESDL |
| | SLENSKQLEEIVKDKISKSAKKDRLLKLFPGEKNSGIFSEFLKLIVGNQADFRKYFNLDEKASLHFSK |
| | ESYDEDLETLLGHIGDDYSDVFLKAKKLYDAILLSGILTVTDNETEAPLSSAMIMRYKEHEEDLALL |
| | KAYIRKISLETYNEVFKDDTKNGYAGYIDGKTNQEDFYVYLKKLLTGLEGADYFLEKINREDLLRK |
| | QRTFDNGSIPYQIHLQEMRAIIDKQAKFYPFLAKNKERIEKILTFRIPYYVGPLARGNSDFAWSIRKR |
| | NEKITPWNFEDVIDKESSAEAFINRMTSFDLYLPEEKVLPKHSLLYETFTVYNELTKVRFIAESMRD |
| | YQFLDSKQKKDIVRLYFKGKRKVTDKDIIDYLHAIDGYDGIELKGIEKQFNSSLSTYHDLLNIINDKE |
| | FLDDSSNEAIIEEIIHTLTIFEDREMIKQRLSKFENIFDKSVLKKLSRRHYTGWGKLSAKLINGIRDEK |
| | SGKTILDYLIDDGVSNRNFMQLIHDDTLSFKKKIQKAQIIGDQDNIKQVVQSLPGSPAIKKGILQSIKI |
| | VDELVKVMGREPESIVVEMARENQYTNQGKSNSQQRLKRLEESLKELGSKILKENVPAKLSKIDNN |
| | ALQNERLYLYYLQNGKDMYTGDDLDIDRLSNYDIDHIIPQAFLKDNSIDNKVLVSSASNRGKSDDV |
| | PSIEVVKARKTFWHQLLKSQLISQRKFDNLTKAERGGLTDDDKAGFIRRQLVETRQITKHVAQLLD |
| | SRFNTKRDDNDKVIRNVKIITLKSSLVSQFRKEFGLYKVREINDYHHAHDAYLNAVVATAILKKYP |
| | KLEPEFVYGDYQKYDVRKYLSTKDNKDLGKATAKMFFYSNLMNFFKKEVRLADGTVVVRPVIEY |
| | SKDTGEIAWNKEKDFATVRKVLSCPQVNIVKKTEVQTGGFSKESILPKRNSDKLIARKKDWDPKK |
| | YGGFDSPTVAYSVLVVAKVEKGKSKKLKSVKELLGITIMERSSFEKNPIDFLEAKGYKEVRKDLIV |
| | KLPKYSLFELENGRKRMLASAGELQKGNELALPSKYVNFLYLASHYEKLKGSPEDNEQKQLFVEQ |
| | HKHYLDEIIEQISEFSKRVILADANLDKVLSAYNKHRDKPIREQAENIIHLFTLTNLGAPAAFKYFDT |
| | TIDRKRYTSTKEVLDATFIHQSITGLYETRIDLSQLGGD |
| 132 | MKKYSIGLDIGTNSVGWAVVDDDYKVPAKKMRVFGDTDRSHIKKNLLGTLLFDSGETAESRRLKR |
| | TARRRYTRRRNRILYLQEIFAEEMNKVDDSFFHRLDESFLTDDDKNFDSHPIFGNKAEEDAYHQKF |
| | PTIYHLRKHLADSTEKADLRLVYLALAHMIKFRGHFLIEGELNAENTDVQKLFEQFVGVYDRTFDD |
| | SHLSEITVDASSILTEKISKSRRLENLIKHYPTEKKNTLFGNLVALALGLQPNFKTNFKLSEDAKLQF |
| | SKDTYEEDLEELLGKIGDEYADLFTSAKNLYDAILLSGILTVDDNSTKAPLSASMIKRYEEHHEDLE |
| | KLKTFIRVNNSDKYHEIFNDKDKNGYAGYIENGVKQDEFYKYLKETLSKIAGSQYFLDKIEREDFL |
| | RKQRTFDNGSIPHQIHLQEMHAILRRQGEYYPFLKENQAKIEKILTFRIPYYVGPLARKDSRFAWAN |
| | YHSDEPITPWNFDEVVDKEKSAEKFITRMTINDLYLPEEKVLPKHSHVYETYAVYNELTKIKYVNE |
| | QGKESFFDANMKQEIFEHVFKENRKVTKDKFLNYLNKEFPEYRIQDLIGLDKENKSFNASLGTYHD |
| | LKKILDKSFLDDKTNEQIIEDIIQTLTLFEDRDMIRQRLQKYSDIFTKQQLKKLERRHYTGWGRLSY |
| | KLINGIRNKENNKTILDYLIDDGYANRNFMQLINDDTLPFKQIIQKSQVVGDIDDIKAVVHDLPGSP |
| | AIKKGILQSVKIVDELVKVMGHNPDNIVIEMARENQTTNRGRSQSQQRLKKLQESLKELGSNILNEE |
| | KPSYIEDKVENSHLQNDKLFLYYIQNGKDMYTGDELDIDHLSDYDIDHIIPQAFIKDDSIDNRVLTSS |
| | AKNRGKSDDVPSLDIVHDRKADWIRLYKSGLISKRKFDNLTKAERGGLTEADKAGFIKRQLVETR |
| | QITKHVAQILDARFNTKRDDNDKVIRDVKVITLKSNLVSQFRKEFGFYKVREINDYHHAHDAYLN |
| | AVVGTALLKKYPKLAPEFVYGEYKKYDIRKFITATDDKATAKYFFYSNLLNFFKTKVRYADGTVI |
| | VRPVIEYSKDTGEIAWNKEKDFATVRKVLSCPQVNIVKKTEVQTGGFSKESILPKRNSDKLIARKKD |
| | WDPKKYGGFDSPTVAYSVLVVAKVEKGKSKKLKSVKELLGITIMERSSFEKNPIDFLEAKGYKEVR |
| | KDLIVKLPKYSLFELENGRKRMLASAGELQKGNELALPSKYVNFLYLASHYEKLKGSPEDNEQKQ |
| | LFVEQHKHYLDEIIEQISEFSKRVILADANLDKVLSAYNKHRDKPIREQAENIIHLFTLTNLGAPAAF |
| | KYFDTTIDRKRYTSTKEVLDATFIHQSITGLYETRIDLSQLGGD |
| 133 | MTKPYSIGLDIGTNSVGWAVITDSYKVPSKKMKVLGNTSKKYVKKNLLGALLFESGITAEGRRLK |
| | RTARRRYTRRRNRILYLQEIFSTEMSKIDESFFQRLDDSFLVPDDKRDSKYPIFGNLVEEKTYHDEFP |
| | TIYHLRKYLADSSKKADLRLVYLALAHMIKYRGHFLIEGDENSKNNDIKKNFQNFLDSYNAIFESD |
| | LSLENSKQLEEIVKDKISKSAKRERVLKLFPDEKSTGLFSEFLKLIVGNQADFKKHFDLEEKAPLQFS |
| | KDTYDEDLENLLGQIGDGFADLFLVAKKLYDAILLSGILTVTDPSTKAPLSASMIERYENHQKDLA |
| | ALKQFIKNNLPEKYDEVFSDQSKDGYAGYIDGKTTQEAFYKYIKNLLSKFEGADYFLDKIEREDFL |
| | RKQRTFDNGSIPHQIHLQEMNAIIRRQGEHYPFLKENKEKIEKILTFRIPYYVGPLARGNRDFAWLTR |
| | NSDQAIRPWNFEEIVDQASSAEDFINKMTNYDLYLPEEKVLPKHSLLYETFAVYNELTKVKFIAEGL |
| | RDYQFLDSGQKKQIVNQLFKEKRKVTEKDIIQFLHNVDGYDGIELKGIEKQFNASLSTYHDLLKIIK |
| | DKEFMDDPKNEEILENIVHTLTIFEDREMIKQRLAQYDSLFDEKVIKALTRRHYTGWGKLSAKLING |
| | IRDKQSQKTILDYLIDDGEINRNFMQLIHDDGLSFKEIIQKAQVFGKTDDVKQVVQELPGSPAIKKGI |
| | LQSIKIVDELVKVMGHNPEHIVVEMARENQFTNQGRRNSQQRYKKIMEAIKNLDSNLNSKILKEYP |
| | TDNQALQNDRLFLYYLQNGKDMYTGEALDINQLSNYDIDHIIPQAFIKDDSLDNRVLTSSKDNRGK |
| | SDNVPSLEIVEKMKGFWQQLLNSKLISERKFNNLTKAERGGLDERDKVGFIKRQLVETRQITKHVA |
| | QILDARFNTEVNEKNQKIRTVKIITLKSNLVSNFRKEFGLYKVREINDYHHAHDAYLNAVVAKAIL |
| | KKYPKLEPEFVYGDYQKYEISRYISRSKISDSIDKATAKYFFYSNLLNFFKEKVRYSNGTVIIRPVIEY |
| | SKDTGEIAWNKEKDFATVRKVLSCPQVNIVKKTEVQTGGFSKESILPKRNSDKLIARKKDWDPKK |
| | YGGFDSPTVAYSVLVVAKVEKGKSKKLKSVKELLGITIMERSSFEKNPIDFLEAKGYKEVRKDLIV |
| | KLPKYSLFELENGRKRMLASAGELQKGNELALPSKYVNFLYLASHYEKLKGSPEDNEQKQLFVEQ |
| | HKHYLDEIIEQISEFSKRVILADANLDKVLSAYNKHRDKPIREQAENIIHLFTLTNLGAPAAFKYFDT |
| | TIDRKRYTSTKEVLDATFIHQSITGLYETRIDLSQLGGD |
| 134 | MTKPYSIGLDIGTNSVGWAVITDNYKVPSKKMKVLGNTSKKYVKKNLLGALLFESGITAEGRRLK |
| | RTARRRYTRRRNRILYLQEIFSTEMAEIDESFFQRLDDSFLVPDDKRDSKYPIFGNLVKEKTYHDEFP |
| | TIYHLRKYLADSSKKADLRLVYLALAHMIKYRGHFLIEGDFNSRNNDIKKNFQEFLDSYNAIFESDL |
| | SLENSKQLEEIVKDKISKSAKRERVLKLFPDEKSTGLFSEFLKLIVGNQADFKKHFDLEEKAPLQFSK |
| | DTYDEDLENLLGQIGDDFADLFLVAKKLYDAILLSGILTVTDPSTKAPLSASMIERYENHQKDLAAL |
| | KQFIQNNLQEKYDEVFSDQSKDGYAGYIDGKTTQEAFYKYIKNLLSKFEGADYFLEKIEREDFLRK |
| | QRTFDNGSIPHQIHLQEMNAIIRRQGEHYPFLKENKEKIEKILTFRIPYYVGPLARGNRDFAWLTRNS |
| | DQAIRPWNFEEVVDKASSAEDFINKMTNYDLYLPEEKVLPKHSLLYETFAVYNELTKVKFIAEGLD |
| | RYQFLDKKQKKDIFDTFFKAEDKRKVTEKDIIHYLHNVDGYDGIELKGIEKQFNASLSTYHDLLKII |
| | KDKEFMDDPKNEEILENIVHTLTIFEDREMIKQRLAQYDSLFDEKVIKALTRRHYTGWGKLSAKLIN |
| | GIYDKQSKKTILDYLIDDDKINRNFMQLINDDGLSFKDIIQKAQVVGKTDDVKQVVQELPGSPAIKK |
| | GILQSIKIVDELVKVMGHAPESIVIEMARENQTTARGKKNSQQRYKRIEDALKNLAPGLDSNILKEN |
| | PTDNVQLQNDRLFLYYLQNGKDMYTGEALDINQLSNYDIDHIIPQAFIKDDSLDNRVLTSSKDNRG |
| | KSDNVPSIEVVQKRKAFWQQLLDSKLISERKENNLTKAERGGLDERDKVGFIKRQLVETRQITKHV |
| | AQILDARFNTEVDENNKKIRKVKIITLKSNLVSNFRKEFELYKVREINDYHHAHDAYLNAVVAKAI |
| | LKKYPKLEPEFVYGDYQKYDIKRYISKSKDPKEIDKATEKYFFYSNLLNFFKEEVHYADGTIIKRENI |
| | EYSKDTGEIAWNKEKDFATVRKVLSCPQVNIVKKTEVQTGGFSKESILPKRNSDKLIARKKDWDPK |
| | KYGGFDSPTVAYSVLVVAKVEKGKSKKLKSVKELLGITIMERSSFEKNPIDFLEAKGYKEVRKDLI |
| | VKLPKYSLFELENGRKRMLASAGELQKGNELALPSKYVNFLYLASHYEKLKGSPEDNEQKQLFVE |
| | QHKHYLDEIIEQISEFSKRVILADANLDKVLSAYNKHRDKPIREQAENIIHLFTLTNLGAPAAFKYFD |
| | TTIDRKRYTSTKEVLDATFIHQSITGLYETRIDLSQLGGD |
| 135 | MKKPYSIGLDIGTNSVGWAVVTDDYKVPAKKMKVLGDTDKSHIKKNLLGTLLFESGETAEGRRLK |
| | RTARRRYTRRRNRILYLQEIFAEEMNKVDDSFFQRLDESFLKDDDKNFDSHPIFGNKAEEDAYHQK |
| | FPTIYHLRQYLADSPEKADLRLVYLALAHIIKYRGHELFEGDLKSENKDVQHLFNDFVEMEDKTVE |
| | GSYLSENLPNVADVLVEKVSKSRRLENVLHYFPNEKKNGLFGNFIALALGLQPNFKTNFKLSEDAK |
| | LQFSKDTYDEDLEELLGKIGDDYADLFVAAKNLYDAILLSGILTVTDPTTKAPLSASMIDRYEKHH |
| | KDLALLKNLIKQNLTQEKYDEVFSDESKNGYAGYISGKVKQEEFYKYLKNTLSKIEGSDYFLDKIE |
| | REDFLRKQRTFDNGSIPHQIHLQEMNAIIRRQGEHYPFLKENKEKIEKILTFRIPYYVGPLARGNRDF |
| | AWLTRNSDQAIRPWNFEEIVDQASSAEDFINKMTNYDLYLPEEKVLPKHSLLYETFAVYNELTKVK |
| | FIAEGLRDYQFLDSGQKKQIVNQLFKEKRKVTEKDIIQYLHNVDGYDGIELKGIEKQFNASLSTYHD |
| | LLKIIKDKEFMDDPKNEEILENIVHTLTIFEDREMIKQRLAQYDSLFDEKVIKALTRRHYTGWGKLS |
| | AKLINGIYDKQSKKTILDYLIDDGEINRNFMQLINDDGLSFKDIIQKAQVVGKTNDVKQVVQELPGS |
| | PAIKKGILQSIKLVDELVKVMGHAPESIVIEMARENQTTARGKKNSQQRYKRIEDALKNLAPGLDS |
| | NILKENPTDNIQLQNDRLFLYYLQNGKDMYTGEALDINQLSNYDIDHIVPQAFIKDDSLDNRVLTSS |
| | KDNRGKSDNVPSIEVVQKRKAFWQQLLDSKLISERKENNLTKAERGGLDERDKVGFIKRQLVETR |
| | QITKHVAQILDARFNTEVDENNQRIRKVKIITLKSNLVSNFRKEFGLYKVREINDYHHAHDAYLNA |
| | VVAKAILKKYPKLEPEFVYGDYQKYDLKRYISRSKDPKDIEKATEKYFFYSNLLNFFKEEVHYADG |
| | TIIKRENIEYSNDTGEIAWNKEKDFATVRKVLSCPQVNIVKKTEVQTGGFSKESILPKRNSDKLIARK |
| | KDWDPKKYGGFDSPTVAYSVLVVAKVEKGKSKKLKSVKELLGITIMERSSFEKNPIDFLEAKGYKE |
| | VRKDLIVKLPKYSLFELENGRKRMLASAGELQKGNELALPSKYVNFLYLASHYEKLKGSPEDNEQ |
| | KQLFVEQHKHYLDEIIEQISEFSKRVILADANLDKVLSAYNKHRDKPIREQAENIIHLFTLTNLGAPA |
| | AFKYFDTTIDRKRYTSTKEVLDATFIHQSITGLYETRIDLSQLGGD |
| 136 | MKKPYSIGLDIGTNSVGWAVITDDYKVPAKKMKVLGNTDKSHIKKNLIGALLFDSGNTAEDRRLK |
| | RTARRRYTRRRNRILYLQEIFAEEMNKVDESFFHRLDDSFLVPEDKRGSKYPIFATLEEEKEYHKKF |
| | PTIYHLRKHLADSKEKTDLRLIYLALAHMIKYRGHFLYEESFDIKNNDIQKIFNEFISIYDNTFEGSSL |
| | SGQNAQVEAIFTDKISKSAKRERVLKLFPDEKSTGLFSEFLKLIVGNQADFKKHFDLEEKAPLQFSK |
| | DTYDEDLENLLGQIGDDFADLFLVAKKLYDAILLSGILTVTDPSTKAPLSASMIERYENHQEDLAAL |
| | KQFIRNNLQEKYDEVFSDSSKDGYAGYIEGKTNQEAFYKYMKKLLTKVEGADYFLEKIKDENFLR |
| | KQRTFDNGSIPHQVHLREMHAIIRRQGEYYPFLKENREKIEKILTFRIPYYVGPLARGKSDFAWMTR |
| | KSDDAIRPWNFEEMVDKEASAMAFIHRMTNNDFYLPEEKVLPKHSLLYEKFAVYNELTKVRYVTE |
| | QGKSFFFDANMKQEIFDKTFKVYRKVTKEKLMDFLGKEFDEFRIVDLMGLDKENKSFNASLGTYH |
| | DLKKIVSKDFLDNPENEDILENVVLTLTLFEDREMIRKRLEKYKDVLTEEQRKKLERRHYTGWGRL |
| | SAKLINGIRDKVTRKTILDYLIDDGTSNRNFMQLIHDDTLSFVDEIRLAQSSGEAEDYRAEVQNLAG |
| | SPAIKKGILQSLKIVDELIEVMGYDPEHIVVEMARENQFTNQGRRNSQQRYKKIKEAIKNLDSNLNS |
| | KILKEYPTNNQALQNDRLFLYYLQNGKDMYTGEALDINQLSSYDIDHIVPQAFIKDDSLDNRVLTS |
| | SKDNRGKSDNVPSLEVVQKRKAFWQQLLDSKLISERKENNLTKAERGGLDERDKVGFIKRQLVET |
| | RQITKHVAQILDARFNTEVNEKGKRIRKVKIITLKSNLVSNFRKEFGLYKVREINDYHHAHDAYLN |
| | AVVAKAILKKYPKLEPEFVYGDYQKYDIKRYISRSKDPKEIEKATQKYFFYSNLLNFFKEEVHYAD |
| | GTIIKRENIEYSKDTGEIAWNKEKDFATVRKVLSCPQVNIVKKTEVQTGGFSKESILPKRNSDKLIAR |
| | KKDWDPKKYGGFDSPTVAYSVLVVAKVEKGKSKKLKSVKELLGITIMERSSFEKNPIDFLEAKGY |
| | KEVRKDLIVKLPKYSLFELENGRKRMLASAGELQKGNELALPSKYVNFLYLASHYEKLKGSPEDN |
| | EQKQLFVEQHKHYLDEIIEQISEFSKRVILADANLDKVLSAYNKHRDKPIREQAENIIHLFTLTNLGA |
| | PAAFKYFDTTIDRKRYTSTKEVLDATFIHQSITGLYETRIDLSQLGGD |
| 137 | MNKPYSIGLDIGTNSVGWAVITDSYKVPSKKMKVLGNTSKKYVKKNLLGALLFESGITAEGRRLK |
| | RTARRRYTRRRNRILYLQEIFSTEMKKIDESFFQRLDDSFLVPEDKRGSKYPIFGNLAQEKTYHEKFP |
| | TIYHLRKYLADSSKKADLRLVYLALAHMIKYRGHFLIEGDFNSRNNDIKKNFQNFLDSYNAIFESDL |
| | SLENSKQLEEIVKDKISKSAKRERVLKLFPDEKSTGLFSEFLKLIVGNQADFKKHFDLEEKAPLQFSK |
| | DTYDEDLENLLGQIGDDFADLFLVAKKLYDAILLSGILTVTDPSTKAPLSASMIERYENHQKDLAAL |
| | KQFIQNNLQEKYDEVFSDQSKDGYAGYIDGKTTQEAFYKYIKNLLSKFEGADYFLEKIEREDFLRK |
| | QRTFDNGSIPHQIHLQEMNAILRRQGEYYPFLKENKEKIEKILTFRIPYYVGPLARGNRDFAWLTRN |
| | SDQAIRPWNFEEVVDQASSAEEFINKMTNYDLYLPEEKVLPKHSLLYETFAVYNELTKVKFIAEGL |
| | RDYQFLDSGQKKQIVNQLFKEKRKVTEKDIIQFLHNVDGYDGIELKGIEKQFNASLSTYHDLLKIIK |
| | DKEFMDDPKNEEILENIVHTLTIFEDREMIKQRLAQYDSLFDEKVIKALTRRHYTGWGKLSAKLING |
| | IRDKQSGKTILDFLIDDDKINRNFMQLINDDGLSFKEIIQKAQVVGKTDDIKQVVQNLPGSPAIKKGI |
| | LQSIKIVDELVKVMGRDPESIVVEMARENQFTNQGRRNSQQRYKKIENAIKNLDKNLNSKILKEYP |
| | TNNQALQNDRLFLYYLQNGKDMYKDEELDINQLSNYDIDHIVPQAFIKDDSLDNRVLTSSKDNRG |
| | KSDNVPSIEVVQKRKGFWQQLLDSKLISERKENNLTKAERGGLDERDKVGFIKRQLVETRQITKHV |
| | AQILDARFNTEVNENNQKIREVKIITLKSNLVSNFRKEFGLYKVREINDYHHAHDAYLNAVVAKAI |
| | LKKYPKLEPEFVYGDYQKYDIKRYISRSKDPKDIEKATEKYFFYSNLLNFFKEEVTYADGTIKKREN |
| | IEYSNDTGEIAWNKEKDFATVRKVLSCPQVNIVKKTEVQTGGFSKESILPKRNSDKLIARKKDWDP |
| | KKYGGFDSPTVAYSVLVVAKVEKGKSKKLKSVKELLGITIMERSSFEKNPIDFLEAKGYKEVRKDL |
| | IVKLPKYSLFELENGRKRMLASAGELQKGNELALPSKYVNFLYLASHYEKLKGSPEDNEQKQLFVE |
| | QHKHYLDEIIEQISEFSKRVILADANLDKVLSAYNKHRDKPIREQAENIIHLFTLTNLGAPAAFKYFD |
| | TTIDRKRYTSTKEVLDATFIHQSITGLYETRIDLSQLGGD |
| 138 | MTKPYSIGLDIGTNSVGWAVITDSYKVPSKKMKVLGNTSKKYVKKNLLGALLFESGITAEGRRLK |
| | RTARRRYTRRRNRILYLQEIFSTEMAKIDESFFQRLDDSFLVPDDKRDSKYPIFGNLVEEKAYHDEF |
| | PTIYHLRKYLADSSKKADLRLVYLALAHMIKYRGHFLIEGDFNSRNNDIKKNFQDFLDSYNAIFESD |
| | LSLENSKQLEEIVKDKISKSAKKDRLLKLFPGEKNSGIFSEFLKLIVGNQADFRKYFNLEDQASLQFS |
| | KESYDEDLETLLGHIGDDYSDVFLKAKKLYDAILLSGILTVTDNETEAPLSSAMIMRYKEHEEDLAL |
| | LKAYIRNISLETYNEVFKDDTKNGYAGYIDGKTNQEDFYVYLKKLLTGLEGADYFLEKINREDLLR |
| | KQRTFDNGSIPYQIHLQEMRAIIDKQAKFYPFLAKNKERIEKILTFRIPYYVGPLARGNSDFAWSIRK |
| | RNEKITPWNFEDVIDKESSAEAFINRMTSFDLYLPEEKVLPKHSLLYETFTVYNELTKVRFIAESMR |
| | DYQFLDSKQKKDIVRLYFKGKRKVTDKDIIDYLHAIDGYDGIELKGIEKQFNSSLSTYHDLLNIIND |
| | KEFLDDSSNEAIIEEIIHTLTIFEDREMIKQRLSKFENIFDKSVLKKLSRRHYTGWGRLSAKLINGIRD |
| | EKSGKTILDYLIDDGVSNRNFMQLIHDDTLSFKKKIQKAQIIGDQDNIKQVVQSLPGSPAIKKGILQSI |
| | KIVDELVKVMGRNPESIVVEMARENQYTNQGKSNSQQRLKRLEESLKDLGSKILKENVPAKLSKID |
| | NNALQNERLYLYYLQNGKDMYTGEELDIDRLSNYDIDHIIPQAFLKDNSIDNKVLVSSASNRGKSD |
| | DVPSLDVVKKRKTFWHQLLKSQLISQRKFDNLTKAERGGLTEADKAGFIQRQLVETRQITKHVAR |
| | LLDARFNTKRDDNDKVIRDVKVITLKSSLVSQFRKDFELYKVREINDFHHAHDAYLNAVVATALL |
| | KKYPKLEPEFVYGDYPRYNSFRERKSATERMLFYSNIMNFFKRVKVHLDSNGKVVVRPVIEYSKD |
| | TGEIAWNKEKDFATVRKVLSCPQVNIVKKTEVQTGGFSKESILPKRNSDKLIARKKDWDPKKYGG |
| | FDSPTVAYSVLVVAKVEKGKSKKLKSVKELLGITIMERSSFEKNPIDFLEAKGYKEVRKDLIVKLPK |
| | YSLFELENGRKRMLASAGELQKGNELALPSKYVNFLYLASHYEKLKGSPEDNEQKQLFVEQHKHY |
| | LDEIIEQISEFSKRVILADANLDKVLSAYNKHRDKPIREQAENIIHLFTLTNLGAPAAFKYFDTTIDRK |
| | RYTSTKEVLDATFIHQSITGLYETRIDLSQLGGD |
| 139 | MTKPYSIGLDIGTNSVGWAVITDSYKVPSKKMKVLGNTSKKYVKKNLLGALLFESGITAEGRRLK |
| | RTARRRYTRRRNRILYLQEIFSTEMAKIDESFFQRLDDSFLVPDDKRDSKYPIFGNLVEEKTYHDEFP |
| | TIYHLRKYLADSSKKADLRLVYLALAHMIKYRGHFLIEGDFNSRNNDIKKNFSNFLDSYNAIFESDL |
| | SLENSKQLEEIVKDKISKSAKRERVLKLFPDEKSTGLFSEFLKLIVGNQAEFKKHFDLEEKAPLQFSK |
| | DTYDEDLENLLGQIGDDFADLFLVAKKLYDAILLSGILTVTDPATKAPLSASMIERYENHQKDLSAL |
| | KQFIKRNLSEKYDEVFSDQSKDGYAGYINGKTTQEAFYKYIKNLLSKLEGADYFLDKIEREDFLRK |
| | QRTFDNGSIPHQIHLQEMNAILRRQGEHYPFLKENKEKIQQILTFRIPYYVGPLARGNRDFAWLTRN |
| | SDQAIRPWNFEEIVDKASSAEDFINKMTNYDLYLPEEKVLPKHSLLYETFAVYNELTKVKFIAEGLR |
| | DYQFLDSGQKKQIVNQLFKEKRKVTEKDIIHYLHNVDGYDGIELKGIEKQFNASLSTYHDLLKIIKD |
| | KEFMDDPKNEEILENIVHTLTIFEDREMIKQRLAQYDSIFDEKVIKALTRRHYTGWGKLSAKLINGI |
| | YDKQSKKTILDYLIDDDKINRNFMQLINDDSLSFKDIIQKAQVVGKTDDVKQVVQELPGSPAIKKGI |
| | LQSIKIVDELVKVMGHAPESIVIEMARENQTTARGKKNSQQRYKRIEDALKNLAPGLDSNILKENPT |
| | DNVQLQNDRLFLYYLQNGKDMYTGEALDINQLSNYDIDHIVPQAFIKDDSLDNRVLTSSKDNRGK |
| | SDNVPSLEVVQKRKAFWQQLLDSKLISQRKFDNLTKAERGGLTEHDKAGFIKRQLVETRQITKHV |
| | AQILDARFNTEVNEKNQKIRKVKIITLKSNLVSNFRKEFRLYKVREINDYHHAHDAYLNAVVAKAI |
| | LKKYPKLEPEFVYGDYQKYDIKRYISRTKDPKAIEKATQKYFFYSNLLNFFKEEVHYADGTIIKREN |
| | IEYSKDTGEIAWNKEKDFATVRKVLSCPQVNIVKKTEVQTGGFSKESILPKRNSDKLIARKKDWDP |
| | KKYGGFDSPTVAYSVLVVAKVEKGKSKKLKSVKELLGITIMERSSFEKNPIDFLEAKGYKEVRKDL |
| | IVKLPKYSLFELENGRKRMLASAGELQKGNELALPSKYVNFLYLASHYEKLKGSPEDNEQKQLFVE |
| | QHKHYLDEIIEQISEFSKRVILADANLDKVLSAYNKHRDKPIREQAENIIHLFTLTNLGAPAAFKYFD |
| | TTIDRKRYTSTKEVLDATFIHQSITGLYETRIDLSQLGGD |
| 140 | MTKPYSIGLDIGTNSVGWAVISDSYKVPSKKMKVLGNTSKKYVKKNLLGALLFESGITAEGRRLKR |
| | TARRRYTRRRNRILYLQEIFATEMSKIDESFFQRLDDSFLVPDDKRDSKYPIFGNLVKEKTYHDEFPT |
| | IYHLRKYLADSSKKADLRLVYLALAHMIKYRGHFLIEGDFNSRNNDIKKNFQNFLDSYNAIFESDLS |
| | LENSKQLEEIVKDKISKSAKRERVLKLFPDEKSTGLFSEFLKLIVGNQADFKKHFDLEEKAPLQFSK |
| | DTYDEDLENLLGQIGDDFADLFLVAKKLYDAILLSGILTVTDPSTKAPLSASMIERYENHQKDLATL |
| | KQFIKTNLPEKYDEVFSDQSKDGYAGYIDGKTTQEAFYKYIKNLLSKLEGADYFLDKIEREDFLRK |
| | QRTFDNGSIPHQIHLQEMNAILRRQGEHYPFLKENKEKIEKILTFRIPYYVGPLARGNRDFAWLTRN |
| | SDQAIRPWNFEEIVDKASSAEDFINKMTNYDLYLPEEKVLPKHSLLYETFAVYNELTKVKFIAEGLR |
| | DYQFLDSGQKKQIVNQLFKEKRKVTEKDIIHYLHNVDGYDGIELKGIEKQFNASLSTYHDLLKIIKD |
| | KEFMDDPKNEEILENIVHTLTIFEDREMIKQRLAQYDSLFDEKVIKALTRRHYTGWGKLSAKLINGI |
| | RDKQSQKTILDYLIDDGEINRNFMQLIHDDGLSFKEIIQKAQVFGKTDDVKQVVQELPGSPAIKKGI |
| | LQSIKIVDELVKVMGKDPESIVVEMARENQFTNQGRRNSQQRYKRIENAIKNLDSNLNSKILKEHPT |
| | DNQALQNDRLFLYYLQNGKDMYTGEELDINQLSSYDIDHIIPQAFIKDDSIDNRVLTSSKDNRGKSD |
| | NVPSLEIVQKRKGFWQQLLNSKLISERKENNLTKAERGGLDERDKVGFIKRQLVETRQITKHVAQIL |
| | DARFNTEVDEKGKRIRKVKIITLKSNLVSNFRKEFGLYKVREINDYHHAHDAYLNAVVAKAILKKY |
| | PKLEPEFVYGDYQKYDIKRYISRSKDPKEIEKATEKYFFYSNLLNFFKEEVHYADGTIIKRENIEYSK |
| | DTGEVAWNKEKDFATVRKVLSCPQVNIVKKTEVQTGGFSKESILPKRNSDKLIARKKDWDPKKYG |
| | GFDSPTVAYSVLVVAKVEKGKSKKLKSVKELLGITIMERSSFEKNPIDFLEAKGYKEVRKDLIVKLP |
| | KYSLFELENGRKRMLASAGELQKGNELALPSKYVNFLYLASHYEKLKGSPEDNEQKQLFVEQHKH |
| | YLDEIIEQISEFSKRVILADANLDKVLSAYNKHRDKPIREQAENIIHLFTLTNLGAPAAFKYFDTTIDR |
| | KRYTSTKEVLDATFIHQSITGLYETRIDLSQLGGD |
| 141 | MKKPYSIGLDIGTNSVGWAVITDDYKVPSKKMKVLGNTDKSHIKKNLIGALLFDSGNTAEDRRLK |
| | RTARRRYTRRRNRILYLQEIFAEEMNKVDESFFHRLDDSFLVPEDKRGSKYPIFATLEEEKEYHKKF |
| | PTIYHLRKHLADSKEKTDLRLIYLALAHMIKYRGHFLYEESFDIKNNDIQKIFNEFISIYDNTFEGSSL |
| | SGQNAQVEAIFTDKISKSAKRERILKLFAYEKPTGLFSEFLKLIVGNQADFKKHFDLEEKAPLQFSK |
| | DTYDEDLENLLGQIGDMYADLFLVAKKLYDAILLSGILTVTDPTTKAPLSASMIERYEEHQKDLAQ |
| | LKEFVNQKLSEKYNEVESDVSKDGYAGYIEGKTTQENFYKFIKKAIEKIEGSDYFLDKINREDLLRK |
| | QRTFDNGSIPHQIHLQEMHAIIRRQAEYYPFLAENQDKIEKILTFRIPYYVGPLARGKSEFAWLNRKS |
| | DEKIRPWNFDEMVDKETSAENFITRMTNYDQYLPDQKVLPKHSLLYEKFAVYNELTKVRYVTEQG |
| | KSFFFDANMKQEIFDGVFKVYRKVTKKKLMDFLAKEFDEFRIVDLMGLDKENKSFNASLGTYHDL |
| | KKIVSKDFLDNPENEEILENVVLTLTLFEDREMIRKRLEKYKDVLTEEQRKKLERRHYTGWGRLSA |
| | KLINGIRDKVTRKTILDYLIDDGTSNRNFMQLIHDDTLSFVDEIRLAQGSGEAEDYRTEVQNLAGSP |
| | AIKKGILQSLKIVDELIEVMGYEPEHIVVEMARENQFTNQGRRNSQQRYNKIKEAIKNLDSNLNSKI |
| | LKEYPTNNQALQNDRLFLYYLQNGKDMYTGEPLDINQLSSYDIDHIVPQAFIKDDSLDNRVLTSSK |
| | DNRGKSDNVPSLEVVEKMKAFWQQLLDSKLISERKYNNLTKAERERGGLNELDKVGFIKRQLVET |
| | RQITKHVAQILDARFNTEVNENNQKIRKVKIITLKSNLVSNFRKEFGLYKVREINDYHHAHDAYLN |
| | AVVAKAILKKYPKLEPEFVYGDYQKYDIKRYISRSRDPKEIEKATEKYFFYSNLLNFFKEEVHYAD |
| | GTIVKRENIEYSNDTGEIAWNKEKDFATVRKVLSCPQVNIVKKTEVQTGGFSKESILPKRNSDKLIA |
| | RKKDWDPKKYGGFDSPTVAYSVLVVAKVEKGKSKKLKSVKELLGITIMERSSFEKNPIDFLEAKG |
| | YKEVRKDLIVKLPKYSLFELENGRKRMLASAGELQKGNELALPSKYVNFLYLASHYEKLKGSPED |
| | NEQKQLFVEQHKHYLDEIIEQISEFSKRVILADANLDKVLSAYNKHRDKPIREQAENIIHLFTLTNLG |
| | APAAFKYFDTTIDRKRYTSTKEVLDATFIHQSITGLYETRIDLSQLGGD |
| 142 | MNKPYSIGLDIGTNSVGWAVITDSYKVPSKKMKVLGNTSKKYVKKNLLGALLFESGITAEGRRLK |
| | RTARRRYTRRRNRILYLQEIFATEMNKVDESFFQRLDDSFLVPDDKRDSKYPIFGNLVEEKTYHDEF |
| | PTIYHLRKYLADSSKKADLRLVYLALAHMIKYRGHFLIEGDFNSRNNDIKKNFQNFLDSYNAIFESD |
| | LSLENSKQLEEIVKDKISKSAKRERVLKLFPDEKSTGLFSEFLKLIVGNQADFKKHFDLEEKAPLQFS |
| | KDTYDEDLENLLGQIGDDFADLFLVAKKLYDAILLSGILTVTDPSTKAPLSASMIERYENHQKDLA |
| | ALKQFVKANLPEKYDEVFSDQSKDGYAGYIDGKTTQEAFYKYIKNLLSKFEGADYFLDKIEREDFL |
| | RKQRTFDNGSIPHQIHLQEMNAILRRQGEHYPFLKDNKEKIEKILTFRIPYYVGPLARGNRDFAWLT |
| | RNSDQAIRPWNFEEIVDKASSAEDFINKMTNYDLYLPEEKVLPKHSLLYETFAVYNELTKVKFIAEG |
| | LRDYQFLDSGQKKQIVNQLFKEKRKVTEKDIIHYLHNVDGYDGIELKGIEKQFNASLSTYHDLLKII |
| | KDKEFMDDPKNEEILENIVHTLTIFEDREMIKQRLEQYDSLFDEKVIKALTRRHYTGWGKLSAKLIN |
| | GIRDKQSQKTILDYLIDDDKINRNFMQLINDDGLSFKEIIQKAQVVGKTDDVKQVVQELPGSPAIKK |
| | GILQSIKIVDELVKVMGHAPENIVIEMARTNQGQKSSQDRKTRIENGVKNLASDLNGNILKEYPTDN |
| | IQLQNDRLFLYYLQNGKDMYTGNPLDINQLSSYDIDHIVPQAFIKDDSLDNRVLTSSKDNRGKSDN |
| | VPSIEIVQKRKAFWQQLLDSKLISERKENNLTKAERGGLDERDKVGFIKRQLVETRQITKHVAQILD |
| | ARFNTEVNEKGQRIRKVKIITLKSNLVSNFRKEFGLYKVREINDYHHAHDAYLNAVVAKAILKKYP |
| | KLEPEFVYGDYQKYDIKRYISRSKEPKDIEKATEKYFFYSNLLNFFKEEVHYADGTIIKRENIEYSKD |
| | TGEIAWNKEKDFATVRKVLSCPQVNIVKKTEVQTGGFSKESILPKRNSDKLIARKKDWDPKKYGG |
| | FDSPTVAYSVLVVAKVEKGKSKKLKSVKELLGITIMERSSFEKNPIDFLEAKGYKEVRKDLIVKLPK |
| | YSLFELENGRKRMLASAGELQKGNELALPSKYVNFLYLASHYEKLKGSPEDNEQKQLFVEQHKHY |
| | LDEIIEQISEFSKRVILADANLDKVLSAYNKHRDKPIREQAENIIHLFTLTNLGAPAAFKYFDTTIDRK |
| | RYTSTKEVLDATFIHQSITGLYETRIDLSQLGGD |
| 143 | MTKPYSIGLDIGTNSVGWAVITDSYKVPSKKMKVLGNTSKKYVKKNLLGALLFESGITAEGRRLK |
| | RTARRRYTRRRNRILYLQEIFSTEMNKIDESFFQRLDDSFLVPDDKRDSKYPIFGNLVEEKTYHDEFP |
| | TIYHLRKYLADSPKKADLRLVYLALAHMIKYRGHFLIEGDFNSRNNDIKKNFQEFLDSYNAIFESDL |
| | SLENSKQLEEIVKDKISKSAKRERVLKLFPDEKSTGLFSEFLKLIVGNQADFKKHFDLEEKAPLQFSK |
| | DTYDEDLENLLGQIGDDFADLFLVAKKLYDAILLSGILTVTDPSTKAPLSASMIDRYENHQKDLAA |
| | LKQFVKANLPEKYDEVFSDQSKDGYAGYIDGKTTQEAFYKYIKNLLNKLEGADYFLDKIEREDFLR |
| | KQRTFDNGSIPHQIHLQEMHAILHRQGEHYPFLKENREKIEKILTFRIPYYVGPLARGNRDFAWLTR |
| | NSDQAIRPWNFEEVVDQASSAEEFINKMTNYDLYLPEEKVLPKHSLLYETFAVYNELTKVKFIAEG |
| | LRDYQFLDSGQKKQIVNQLFKEKRKVTEKDIIHYLHNVDGYDGIELKGIEKQFNASLSTYHDLLKII |
| | KDKEFMDDAKNEAILENIVHTLTIFEDREMIKQRLAQYNSLFDEKVIKALTRRHYTGWGRLSAKLI |
| | NGIRDKQSKKTILDYLIDDDKINRNFMQLIHDDGLSFKEIIQKAQVGSHTDDLKQVVQELAGSPAIK |
| | KGILQSLKIVDELIEVMGYDPEHIVVEMARENQFTNQGRRNSQQRYKKIEDAIKNLDSNLNSKILKE |
| | YPTNNQALQNDRLFLYYLQNGKDMYTGEELDINQLSNYDIDHIVPQAFIKDDSLDNRVLTSSKDNR |
| | GKSDNVPSIEVVQKRKAFWQQLLDSKLISERKENNLTKAERGGLDERDKVGFIKRQLVETRQITKH |
| | VAQILDSRENTEVNDKGQKIRKVKIVTLKSNLVSNFRKEFGLYKVREINDYHHAHDAYLNAVVAK |
| | AILKKYPKLEPEFVYGDYQKYDIKRYISRSKDPKDIEKATQKYFFYSNLLNFFKEEVHYADGTIIKR |
| | ENIEYSKDTGEIAWNKEKDFATVRKVLSCPQVNIVKKTEVQTGGFSKESILPKRNSDKLIARKKDW |
| | DPKKYGGFDSPTVAYSVLVVAKVEKGKSKKLKSVKELLGITIMERSSFEKNPIDFLEAKGYKEVRK |
| | DLIVKLPKYSLFELENGRKRMLASAGELQKGNELALPSKYVNFLYLASHYEKLKGSPEDNEQKQLF |
| | VEQHKHYLDEIIEQISEFSKRVILADANLDKVLSAYNKHRDKPIREQAENIIHLFTLTNLGAPAAFKY |
| | FDTTIDRKRYTSTKEVLDATFIHQSITGLYETRIDLSQLGGD |
| 144 | MTKPYSIGLDIGTNSVGWAVITDSYKVPSKKMKVLGNTSKKYVKKNLLGALLFESGITAEGRRLK |
| | RTARRRYTRRRNRILYLQEIFSTEMAKIDESFFQRLDDSFLVPDDKRDSKYPIFGNLVEEKTYHEKFP |
| | TIYHLRKYLADSSKKADLRLVYLALAHMIKYRGHFLIEGDFNSRNNDIKKNFQNFLDSYNAIFESDL |
| | SLENSKQLEEIVKDKISKSAKRERVLKLFPDEKSTGLFSEFLKLIVGNQADFKKHFDLEEKAPLQFSK |
| | DTYDEDLENLLGQIGDDFADLFLVAKKLYDAILLSGILTVTDPSTKAPLSASMIERYENHQKDLAAL |
| | KQFVKANLPEKYDEVFSDQSKDGYAGYIDGKTTQEAFYKYIKNLLSKFEGADYFLEKIEREDFLRK |
| | QRTFDNGSIPHQIHLQEMNAILRRQGEHYPFLKENKEKIEKILTFRIPYYVGPLARGNRDFAWLTRN |
| | SDQAIRPWNFEEIVDQASSAEDFINKMTNYDLYLPEEKVLPKHSLLYETFAVYNELTKVKFIAEGLR |
| | DYQFLDSGQKKQIVNQLFKEKRKVTEKDIIQFLHNVDGYDGIELKGIEKQFNASLSTYHDLLKIIKD |
| | KEFMDDPKNEEILENIVHTLTIFEDREMIKQRLAQYDSLFDEKVIKALTRRHYTGWGKLSAKLINGI |
| | RDKQSNKTILDYLIDDDKINRNFMQLINDDGLSFKDIIQKAQVVGKTDDLKQVVQELPGSPAIKKGI |
| | LQSIKIVDELVKVMGHAPESIVIEMARENQTTARGKKNSQQRYKRIEEALKNLAPGLDSKILKEHPT |
| | DNTQLQNDKLFLYYLQNGRDMYIDEELDINRLSDYDIDHIVPQAFIKDDSLDNRVLTSSKDNRGKS |
| | DNVPSLEVVQKRKGFWRQLLNSKLISKRKFDNLTKAERGGLTENDKAGFIKRQLVETRQITKHVA |
| | QILDARFNTEVDENNKKIRKVKIITLKSNLVSNFRKEFELYKVREINDYHHAHDAYLNAVVAKAIL |
| | KKYPKLEPEFVYGDYQKYDIKRYISRSKDPKEVEKATEKYFFYSNLLNFFKEEVHYADGTIIKRENI |
| | EYSKDTGEIAWNKEKDFATVRKVLSCPQVNIVKKTEVQTGGFSKESILPKRNSDKLIARKKDWDPK |
| | KYGGFDSPTVAYSVLVVAKVEKGKSKKLKSVKELLGITIMERSSFEKNPIDFLEAKGYKEVRKDLI |
| | VKLPKYSLFELENGRKRMLASAGELQKGNELALPSKYVNFLYLASHYEKLKGSPEDNEQKQLFVE |
| | QHKHYLDEIIEQISEFSKRVILADANLDKVLSAYNKHRDKPIREQAENIIHLFTLTNLGAPAAFKYFD |
| | TTIDRKRYTSTKEVLDATFIHQSITGLYETRIDLSQLGGD |
| 145 | MNKPYSIGLDIGTNSVGWAVITDDYKVPSKKMKVLGNTSKKYVKKNLLGALLFESGITAEGRRLK |
| | RTARRRYTRRRNRILYLQEIFEKGMSEIDESFFQRLDDSFLVPDDKRDSKYPIFGNLVKEKTYHDEF |
| | PTIYHLRKYLADSSKKADLRLVYLALAHMIKYRGHFLIEGDFNSRNNDIKKNFQQFLDSYNAIFESD |
| | LSLENSKQLEEIVKDKISKSAKRERVLKLFPDEKSTGLFSEFLKLIVGNQADFKKHFDLEEKAPLQFS |
| | KDTYDEDLENLLGQIGDDFADLFLVAKKLYDAILLSGILTVTDPSTKAPLSASMIERYENHQKDLAT |
| | LKQFVKTNLPEKYDEVFSDQSKDGYAGYIDGKTTQEAFYKYIKNLLSKFEGSDYFLDKIEREDFLR |
| | KQRTFDNGSIPHQIHLQEMNAIIRRQGEHYPFLKENREKIEKILTFRIPYYVGPLARGNRDFAWLTRN |
| | SDQAIRPWNFEEIVDKASSAEEFINKMTNYDLYLPEEKVLPKHSLLYETFAVYNELTKVKFIAEGLR |
| | DYQFLDSGQKKQIVNQLFKEKRKVTEKDIIQFLHNVDGYDGIELKGIEKQFNASLSTYHDLLKIIRD |
| | KEFMDDPKNEEILENVVHTLTIFEDREMIKQRLAQYDSLFDEKVIKALSRRHYTGWGRLSAKLINGI |
| | RDKQSQKTILDYLIDDGEINRNFMQLIHDDGLSFKEIIQKAQVVSHSDDLKQVVQELPGSPAIKKGIL |
| | QSIKIVDELVKVMGHNPESIVIEMARENQTTNQGRRNSQQRYKKINEAIKNLDSNLNSKILKEYPTN |
| | NQALQNDRLFLYYLQNGKDMYTGEALDINQLSNYDIDHIIPQAFIKDDSLDNRVLTSSKDNRGKSD |
| | NVPSIEVVEKMKGFWQQLLDSKLISERKENNLTKAERGGLDERDKVGFIKRQLVETRQITKHVAQL |
| | LDARFNVEVNDKKQRIRNVKIITLKSNLVSNFRKEFGLYKVREINDYHHAHDAYLNAVVAKAILK |
| | KYPKLEPEFVYGDYQKYDVRKFIKSKSEQEIGKATAKKFFYSNLLNFFKKEVRLADGTVIVRPVVE |
| | YSKDTGEIAWNKEKDFATVRKVLSCPQVNIVKKTEVQTGGFSKESILPKRNSDKLIARKKDWDPK |
| | KYGGFDSPTVAYSVLVVAKVEKGKSKKLKSVKELLGITIMERSSFEKNPIDFLEAKGYKEVRKDLI |
| | VKLPKYSLFELENGRKRMLASAGELQKGNELALPSKYVNFLYLASHYEKLKGSPEDNEQKQLFVE |
| | QHKHYLDEIIEQISEFSKRVILADANLDKVLSAYNKHRDKPIREQAENIIHLFTLTNLGAPAAFKYFD |
| | TTIDRKRYTSTKEVLDATFIHQSITGLYETRIDLSQLGGD |
| 146 | MKKPYSIGLDIGTNSVGWAVVTDDYKVPAKKMKVLGNTDKSHIKKNLLGALLFDSGNTAEDRRL |
| | KRTARRRYTRRRNRILYLQEIFAEEMNKVDESFFHRLDDSFLVPEDKRGSKYPIFATLEEEKEYHKK |
| | FPTIYHLRKHLADSKEKTDLRLIYLALAHMIKYRGHFLYEESFDIKNNDIQKIFNEFISIYDNTFEGSS |
| | LSGQNAQVEAIFTDKISKSAKRERVLKLFPDEKSTGLFSEFLKLIVGNQADFKKHFDLEEKAPLQFS |
| | KDTYDEDLENLLGQIGDDFADLFLVAKKLYDAILLSGILTVKDLSTKAPLSASMIERYENHQEDLK |
| | QLKRFVNQKLSEKYNEVESDDSKDGYAGYIEGKTNQEGFYKYIKNLIEKLEGADYFLEKINREDLL |
| | RKQRTFDNGSIPHQIHLQEMHAIIRRQAEFYPFLAENQDKIEKILTFRIPYYVGPLARGKSEFAWLNR |
| | KSDEKIRPWNFDEMVDKETSAENFITRMTNYDQYLPDQKVLPKHSLLYEKFAVYNELTKVRYVTE |
| | QGKSFFFDANMKQEIFDGVFKVYRKVTKEKLLDFLDKEFDEFRIVDLIGLDKENKAFNASLGTYHD |
| | LKKIVSKDFLDNPENEDILENVVLTLTLFEDREMIRKRLEKYKDVLTEEQRKKLERRHYTGWGRLS |
| | AKLINGIRNKTTRKTILDYLIDDGTSNRNFMQLIHDDTLSFVDEIRLAQGSGEAEDYRAEVQNLAGS |
| | PAIKKGILQSLKIVDELIEVMGYDPEHIVVEMARENQFTNQGRRNSQQRYKKIKDAIKNLDSNLNSK |
| | ILKEYPTNNQALQNDRLFLYYLQNGKDMYTGKELDINQLSNYDIDHIVPQAFIKDDSLDNRVLTSS |
| | KDNRGKSDNVPSLEVVQKRKAFWQQLLDSKLISERKYNNLTKAERERGGLNELDKVGFIKRQLVE |
| | TRQITKHVAQILDARFNTEVDENGKRIRKVKIITLKSNLVSNFRKEFGLYKVREINDYHHAHDAYL |
| | NAVVAKAILKKYPKLEPEFVYGDYQKYDIKRYISRSKDPKDIEKATEKYFFYSNLLNFFKEEVHYA |
| | DGTIIKRENIEYSKDTGEIAWNKEKDFATVRKVLSCPQVNIVKKTEVQTGGFSKESILPKRNSDKLIA |
| | RKKDWDPKKYGGFDSPTVAYSVLVVAKVEKGKSKKLKSVKELLGITIMERSSFEKNPIDFLEAKG |
| | YKEVRKDLIVKLPKYSLFELENGRKRMLASAGELQKGNELALPSKYVNFLYLASHYEKLKGSPED |
| | NEQKQLFVEQHKHYLDEIIEQISEFSKRVILADANLDKVLSAYNKHRDKPIREQAENIIHLFTLTNLG |
| | APAAFKYFDTTIDRKRYTSTKEVLDATFIHQSITGLYETRIDLSQLGGD |
| 147 | MTKPYSIGLDIGTNSVGWAVITDSYKVPSKKMKVLGNTSKKYVKKNLLGALLFESGITAEGRRLK |
| | RTARRRYTRRRNRILYLQEIFSTEMAKIDESFFQRLDDSFLVPDDKRDSKYPIFGNLVKEKTYHDEF |
| | PTIYHLRKYLADSSKKADLRLVYLALAHMIKYRGHFLIEGDFNSRNNDIKKNFQDFLDSYNAIFESD |
| | LSLENSKQLEEIVKDKISKSAKKDRLLKLFPGEKNSGIFSEFLKLIVGNQADFRKYFNLDEKASLHES |
| | KESYDEDLETLLGHIGDDYSDVFLKAKKLYDAILLSGILTVTDNETEAPLSSAMIMRYKEHEEDLAL |
| | LKAYIRKISLETYNEVFKDDTKNGYAGYIDGKTNQEDFYVYLKKLLTGLEGADYFLEKINREDLLR |
| | KQRTFDNGSIPYQIHLQEMRAIIDKQAKFYPFLAKNKERIEKILTFRIPYYVGPLARGNSDFAWSIRK |
| | RNEKITPWNFEDVIDKESSAEAFINRMTSFDLYLPEEKVLPKHSLLYETFTVYNELTKVRFIAESMR |
| | DYQFLDSKQKKDIVRLYFKGKRKVTDKDIIDYLHAIDGYDGIELKGIEKQFNSSLSTYHDLLKIIKD |
| | KEFLDDASNEAIIEEIIHTLTIFEDREMIKQRLSKFENIFDKSVLKKLSRRHYTGWGKLSAKLINGIRD |
| | EKSGKTILDYLIDDGVSNRNFMQLIHDDTLSFKKKIQKAQIIGDQDNIKQVVQSLPGSPAIKKGILQSI |
| | KIVDELVKVMGRNPESIVVEMARENQYTNQGKSNSQQRLKRLEESLKELGSKILKENVPAKLSKID |
| | NNALQNERLYLYYLQNGKDMYTGDDLDIDRLSNYDIDHIIPQAFLKDNSIDNKVLVSSASNRGKSD |
| | DVPSLDVVKKRKTFWHQLLKSKLISQRKFDNLTKAERGGLTEDDKAGFIQRQLVETRQITKHVARI |
| | LDERFNNELDENGKRIRKVKIITLKSSLVSQFRKEFGLYKVREINDYHHAHDAYLNAVVATAILKK |
| | YPKLEPEFVYGEYKKYEVKSTYGEKKEYVREIVRSKTDSSLGKATAKMFFYSNLMNFFKKEVRLS |
| | DGTVIVRPVIEYSKDTGEIAWNKEKDFATVRKVLSCPQVNIVKKTEVQTGGFSKESILPKRNSDKLI |
| | ARKKDWDPKKYGGFDSPTVAYSVLVVAKVEKGKSKKLKSVKELLGITIMERSSFEKNPIDFLEAK |
| | GYKEVRKDLIVKLPKYSLFELENGRKRMLASAGELQKGNELALPSKYVNFLYLASHYEKLKGSPE |
| | DNEQKQLFVEQHKHYLDEIIEQISEFSKRVILADANLDKVLSAYNKHRDKPIREQAENIIHLFTLTNL |
| | GAPAAFKYFDTTIDRKRYTSTKEVLDATFIHQSITGLYETRIDLSQLGGD |
| 148 | MKKYSIGLDIGTNSVGWAVVDDDYKVPAKKMRVFGDTDRSHIKKNLLGTLLFDSGETAENRRLK |
| | RTARRRYTRRRNRILYLQEIFAKEMAKVDESFFHRLDESFLTDDDKTFDSHPIFGNKAEEDAYHQK |
| | FPTIYHLRKHLADSTEKADLRLVYLALAHMIKFRGHFLIEGELNAENTDVQKLFAQFVGVYDRTFD |
| | DSHLSEITVDASSILTEKISKSRRLENLIKHYPTEKKNTLFGNLVALALGLQPNFKTNFKLSEDAKLQ |
| | FSKDTYEEDLEELLGKIGDEYADLFTSAKNLYDAILLSGILTVDDNSTKAPLSASMIKRYVEHHEDL |
| | EKLKEFIKANKPDFYHDIFKDKDKNGYAGYIENGVKQDEFYKYLKNTLSKIAGSDYFLDKIEREDF |
| | LRKQRTFDNGSIPHQIHLQEMHAILRRQGEYYPFLKENQAKIEKILTFRIPYYVGPLARKDSRFAWA |
| | NYHSDEPITPWNFDEVVDKEKSAEKFITRMTLNDLYLPEEKVLPKHSLVYETYTVYNELTKIKYVN |
| | EQGKETFFDANMKQEIFEHVFKENRKVTKEKFLNYLNKEFPEYRIQDLIGLDKENKSFNASLGTYH |
| | DLKKILDKSFLDDKTNEQIIEDIIQTLTLFEDRDMIRQRLQKYSDIFTKQQLKKLERRHYTGWGRLS |
| | YKLINGIRNKENNKTILDYLIDDGYANRNFMQLINDDTLPFKQIIQKSQVVGDIDDIKAVVHDLPGS |
| | PAIKKGILQSVKIVDELVKVMGHNPDNIVIEMARENQTTNRGRTQSQQRLKKLQDSLKELGSNILN |
| | EEKPSYVEGKVENNHLQNDKLFLYYIQNGKDMYTGEELDIDHLSDYDIDHIIPQAFIKDDSIDNRVL |
| | TSSAKNRGKSDDVPSLDIVHDRKADWIRLYKSGLISKRKFDNLTKAERGGLTEADKAGFIKRQLVE |
| | TRQITKHVAQILDARFNTKRDENDKVIRDVKVITLKSNLVSQFRKDFQFYKVREINDYHHAHDAYL |
| | NAVVGTALLKKYPKLAPEFVYGEYKKYDIRKFITNSSDKATAKYFFYSNLLNFFKTKVRYADGTVI |
| | VRPVIEYSKDTGEIAWNKEKDFATVRKVLSCPQVNIVKKTEVQTGGFSKESILPKRNSDKLIARKKD |
| | WDPKKYGGFDSPTVAYSVLVVAKVEKGKSKKLKSVKELLGITIMERSSFEKNPIDFLEAKGYKEVR |
| | KDLIVKLPKYSLFELENGRKRMLASAGELQKGNELALPSKYVNFLYLASHYEKLKGSPEDNEQKQ |
| | LFVEQHKHYLDEIIEQISEFSKRVILADANLDKVLSAYNKHRDKPIREQAENIIHLFTLTNLGAPAAF |
| | KYFDTTIDRKRYTSTKEVLDATFIHQSITGLYETRIDLSQLGGD |
| 149 | MKKYSIGLDIGTNSVGWAVVDDDYKVPAKKMRVFGDTDRSHIKKNLLGTLLFDSGETAESRRLKR |
| | TARRRYTRRRNRILYLQEIFAEEMNKVDDSFFHRLDESFLTDDDKTFDSHPIFGNKAEEDAYHQKFP |
| | TIYHLRKHLADSTEKADLRLVYLALAHMIKFRGHFLIEGELNAENTDVQKLFAQFVGVYDRTFDDS |
| | HLSEITVDASSILTEKISKSRRLENLIKHYPTEKKNTLFGNLVALALGLQPNFKTNFKLSEDAKLQFS |
| | KDTYEEDLEELLGKIGDDYADLFTSAKNLYDAILLSGILTVDDESTKAPLSASMVKRYAEHHEDLE |
| | KLKEFIKANKPELYHDIFKDETKNGYAGYIENGVKQDEFYKYLKTTLSKIEGSDYFLDKIEREDFLR |
| | KQRTFDNGSIPHQIHLQEMHAILRRQGDYYPFLKENQAKIEKILTFRIPYYVGPLARKDSRFAWANY |
| | HSDEPITPWNFDEVVDKEKSAEKFITRMTINDLYLPEEKVLPKHSHVYETYAVYNELTKIKYVNEQ |
| | GKSFFFDANMKQEIFEHVFKENRKVTKDKFLNYLNKEFPEYRIQDLIGLDKENKSFNASLGTYHDL |
| | KKILDKSFLDDKTNEQIIEDIVLTLTLFEDRDMIRQRLQKYSDIFTPQQLKKLERRHYTGWGRLSYK |
| | LINGIRNKENNKTILDFLIDDGHANRNFMQLINDDSLSFKTIIQEAQVVGDVDDIEAVVHDLPGSPAI |
| | KKGILQSVKIVDELVKVMGHNPDNIVIEMARENQTTNRGRSQSQQRLKKLQDSLKELGSNILNEEK |
| | PSYIEDKVENSHLQNDKLFLYYIQNGKDMYTGEELDIDHLSDYDIDHIIPQAFIKDDSIDNRVLTSSA |
| | KNRGKSDDVPSLDIVHDRKADWIRLYKSGLISKRKFDNLTKAERGGLTEADKAGFIKRQLVETRQI |
| | TKHVAQILDARFNTERDENDKVIRDVKVITLKSNLVSQFRKDFQFYKVREINDYHHAHDAYLNAV |
| | VGTALLKKYPKLAPEFVYGEYKKYDIRKFITNSSDKATAKYFFYSNLLNFFKTKVRYADGTVIVRP |
| | VIEYSKDTGEIAWNKEKDFATVRKVLSCPQVNIVKKTEVQTGGFSKESILPKRNSDKLIARKKDWD |
| | PKKYGGFDSPTVAYSVLVVAKVEKGKSKKLKSVKELLGITIMERSSFEKNPIDFLEAKGYKEVRKD |
| | LIVKLPKYSLFELENGRKRMLASAGELQKGNELALPSKYVNFLYLASHYEKLKGSPEDNEQKQLFV |
| | EQHKHYLDEIIEQISEFSKRVILADANLDKVLSAYNKHRDKPIREQAENIIHLFTLTNLGAPAAFKYF |
| | DTTIDRKRYTSTKEVLDATFIHQSITGLYETRIDLSQLGGD |
| 150 | MKKPYSIGLDIGTNSVGWAVVTDDYKVPAKKMKVLGNTDKSHIKKNLLGALLFDSGETAEATRL |
| | KRTARRRYTRRRNRILYLQEIFAKEMAKVDESFFHRLDESFLTDDDKNFDSHPIFGNKAEEDAYHQ |
| | KFPTIYHLRKHLADSTEKADLRLVYLALAHMIKFRGHFLIEGQLNAENTDVQTLFNDFVEVYDKTV |
| | EESHLSEITVDALSILTEKVSKSRRLENLIKYYPTEKKNTLFGNLIALALGLQPNFKTNFKLSEDAKL |
| | QFSKDTYEEDLEELLGKIGDEYADLFTSAKNLYDAILLSGILTVDDESTKAPLSASMIKRYAEHHED |
| | LEKLKEFIKANKPELYHDIFKDETKNGYAGYIENGVKQDEFYKYLKNTLTKIEGSDYFLDKIEREDF |
| | LRKQRTFDNGSIPHQIHLQEMHAILRRQGDYYPFLKENQDRIEKILTFRIPYYVGPLARKDSRFAWA |
| | NYHSDEPITPWNFDEVVDKEKSAEKFITRMTLNDLYLPEEKVLPKHSHVYETYAVYNELTKIKYVN |
| | EQGKESFFDANMKQEIFEHVFKENRKVTKEKFLNYLNKEFPEYRIQDLIGLDKENKSFNASLGTYH |
| | DLKKILDKSFLDDKTNEQIIEDIIQTLTLFEDRDMIRQRLQKYSDIFTKQQLKKLERRHYTGWGRLS |
| | YKLINGIRNKENNKTILDYLIDDGYANRNFMQLINDDTLPFKQIIQKSQVIGDIDDIKAVVHDLPGSP |
| | AIKKGILQSVKIVDELVKVMGDNPDNIVIEMARENQTTNRGRTQSQQRLKKLQESLKELGSNILNE |
| | EKPSYIEDKVENSHLQNDKLFLYYIQNGKDMYTGEELDIDHLSDYDIDHIIPQAFIKDDSIDNRVLTS |
| | SAKNRGKSDDVPSLDIVHDRKADWIRLYKSGLISKRKFDNLTKAERGGLTEADKAGFIKRQLVETR |
| | QITKHVAQILDARFNTERDENDKVIRDVKVITLKSNLVSQFRKDFQFYKVREINDYHHAHDAYLNA |
| | VVGTALLKKYPKLAPEFVYGEYKKYDIRKFITASGDKATAKYFFYSNLLNFFKTKVRYADGTVIVR |
| | PVIEYSKDTGEIAWNKEKDFATVRKVLSCPQVNIVKKTEVQTGGFSKESILPKRNSDKLIARKKDW |
| | DPKKYGGFDSPTVAYSVLVVAKVEKGKSKKLKSVKELLGITIMERSSFEKNPIDFLEAKGYKEVRK |
| | DLIVKLPKYSLFELENGRKRMLASAGELQKGNELALPSKYVNFLYLASHYEKLKGSPEDNEQKQLF |
| | VEQHKHYLDEIIEQISEFSKRVILADANLDKVLSAYNKHRDKPIREQAENIIHLFTLTNLGAPAAFKY |
| | FDTTIDRKRYTSTKEVLDATFIHQSITGLYETRIDLSQLGGD |
| 151 | MSNKPYSIGLDIGTNSVGWAVVTDDYKVPAKKMKVLGDTDKSHIKKNLLGALLFEGGETAEGRR |
| | LKRTARRRYTRRRNRILYLQEIFAEEMNKVDESFFHRLDDSFLVPEDKRGSKYPIFATLEEEKEYHK |
| | KFPTIYHLRKHLADSKEKTDLRLIYLALAHMIKYRGHFLYEESFDIKNNDIQKIFNEFISIYDNTFEGS |
| | SLSGQNAQVEAIFTDKISKSAKRERVLKLFPDEKSTGLFSEFLKLIVGNQADFKKHFDLEEKAPLQFS |
| | KDTYDEDLENLLGQIGDDFADLFLVAKKLYDAILLSGILTVTDPSTKAPLSASMIQRYEEHQEDLKK |
| | LKQFIKRNIPEKYDEVFSDQSKNGYAGYIDGKTTQEEFYKYLKEKLTSVKGADYFLEKIKNEDFLR |
| | KQRTFDNGVIPYQVHLSEMRAILHRQGEYYPFLKENKEKIEKILTFRIPYYVGPLARGNRDFAWLTR |
| | NSDEAIRPWNFEEVVDKASSAEDFINKMTNYDLYLPEEKVLPKHSLLYEKFAVYNELTKVRYVTE |
| | QGKSFFFDANMKQEIFDKTFKVYRKVTKEKLMDFLGKEFDEFRIVDLMGLDKDNKSFNASLGTYH |
| | DLKKIVSKDFLDNPENEDILENVVLTLTLFEDREMIRKRLEKYKDVLTEEQRKKLERRHYTGWGRL |
| | SAKLINGIRDKVTRKTILDYLIDDGTSNRNFMQLIHDDTLSFVDEIRLAQGSGEAEDYRTEVQNLAG |
| | SPAIKKGILQSLKIVDELIEVMGYDPEHIVVEMARENQFTNQGRRNSQQRYKKIENAIKNLDSNLNS |
| | KILKEYPTNNQALQNDRLFLYYLQNGKDMYTGEALDINQLSNYDIDHIIPQAFIKDDSLDNRVLTSS |
| | KDNRGKSDNVPSLEVVEKMKAFWQQLLDSKLISERKYNNLTKAERERGGLNELDKVGFIKRQLVE |
| | TRQITKHVAQILDARFNTEVNEKNQKIRKVKIITLKSNLVSNFRKEFGLYKVREINDYHHAHDAYL |
| | NAVVAKAILKKYPKLEPEFVYGDYQKYDIKRYISRSKDPKDIEKATEKYFFYSNLLNFFKEEVHYA |
| | DGTIIKRENIEYSKDTGEIAWNKEKDFATVRKVLSCPQVNIVKKTEVQTGGFSKESILPKRNSDKLIA |
| | RKKDWDPKKYGGFDSPTVAYSVLVVAKVEKGKSKKLKSVKELLGITIMERSSFEKNPIDFLEAKG |
| | YKEVRKDLIVKLPKYSLFELENGRKRMLASAGELQKGNELALPSKYVNFLYLASHYEKLKGSPED |
| | NEQKQLFVEQHKHYLDEIIEQISEFSKRVILADANLDKVLSAYNKHRDKPIREQAENIIHLFTLTNLG |
| | APAAFKYFDTTIDRKRYTSTKEVLDATFIHQSITGLYETRIDLSQLGGD |
| 152 | MKKSYSIGLDIGTNSVGWAVVDDDYKVPAKKMRVFGDTDRSHIKKNLLGTLLFDSGETAESRRLK |
| | RTARRRYTRRRNRILYLQEIFAEEMNKVDDSFFHRLDESFLTDDDKNFDSHPIFGNKAEEDAYHQK |
| | FPTIYHLRKHLADSTEKADLRLVYLALAHMIKFRGHFLIEGQLNAENTDVQTLFNDFVEVYDKTVE |
| | ESHLSEITVDALSILTEKVSKSRRLENLIKYYPTEKKNTLFGNLIALALGLQPNFKTNFKLSEDAKLQ |
| | FSKDTYEEDLEELLGKIGDEYADLFTSAKNLYDAILLSGILTVDDNSTKAPLSASMLKRYVEHHEDL |
| | EKLKEFIKANKSELYHDIFKDENKNGYAGYIENGVKQDEFYKYLKNTLSKIAGSDYFLDKIEREDFL |
| | RKQRTFDNGSIPHQIHLQEMHAILRRQGDYYPFLKENQDRIEKILTFRIPYYVGPLARKDSRFAWAN |
| | YHSDEPITPWNFDEVVDKEKSAEKFITRMTLNDLYLPEEKVLPKHSHVYETYAVYNELTKIKYVNE |
| | QGKSFFFDANMKQEIFEHVFKENRKVTKDKFLNYLNKEFPEYRIQDLIGLDKENKSFNASLGTYHD |
| | LKKILDKSFLDDKTNEQIIEDIIQTLTLFEDRDMIRQRLQKYSDIFTKQQLKKLERRHYTGWGRLSY |
| | KLINGIRNKENNKTILDYLIDDGYANRNFMQLINDDTLPFKQIIQEAQVVGDVDDIEAVVHDLPGSP |
| | AIKKGILQSVKIVDELVKVMGHNPDNIVIEMARENQTTNRGRTQSQQRLKKLQESLKELGSNILNE |
| | EKPSYVEGKVENNQLQNDRLFLYYIQNGKDMYTGEELDIDHLSDYDIDHIIPQAFIKDDSIDNRVLT |
| | SSAKNRGKSDDVPSLDIVHDRKADWIRLYKSGLISKRKFDNLTKAERGGLTEADKAGFIKRQLVET |
| | RQITKHVAQILDSRFNTKRDENDKVIRDVKVITLKSNLVSQFRKDFQFYKVREINDYHHAHDAYLN |
| | AVVGTALLKKYPKLAPEFVYGEYKKYDIRKFITASGDKATAKYFFYSNLLNFFKTKVRYADGTVIV |
| | RPVIEYSKDTGEIAWNKEKDFATVRKVLSCPQVNIVKKTEVQTGGFSKESILPKRNSDKLIARKKD |
| | WDPKKYGGFDSPTVAYSVLVVAKVEKGKSKKLKSVKELLGITIMERSSFEKNPIDFLEAKGYKEVR |
| | KDLIVKLPKYSLFELENGRKRMLASAGELQKGNELALPSKYVNFLYLASHYEKLKGSPEDNEQKQ |
| | LFVEQHKHYLDEIIEQISEFSKRVILADANLDKVLSAYNKHRDKPIREQAENIIHLFTLTNLGAPAAF |
| | KYFDTTIDRKRYTSTKEVLDATFIHQSITGLYETRIDLSQLGGD |
| 153 | MKKPYSIGLDIGTNSVGWAVITDDYKVPSKKMKVLGNTDKSHIKKNLIGALLFDSGNTAEDRRLK |
| | RTARRRYTRRRNRILYLQEIFSEEMNKVDESFFHRLDDSFLVPEDKRGSKYPIFATLEEEKEYHKKF |
| | PTIYHLRKHLADSKEKTDLRLIYLALAHMIKYRGHFLYEESFDIKNNDIQKIFNEFISIYDNTFEGSSL |
| | SGQNAQVEAIFTDKISKSAKRERVLRLFPDEKSTGLFSEFLKLIVGNQADFKKHFELEEKAPLQFSK |
| | DTYDEDLEELLGKIGDDYADLFVAAKKLYDAILLSGILTVTDSTTKAPLSSSMVNRYEEHQKDLAL |
| | LKQFVRTNLPEKYAEVFSDASKDGYAGYIEGKTTQENFYKFIKKAIEKIEGSDYFLDKIEREDFLRK |
| | QRTFDNGSIPHQIHLQEMHAIIRRQAEFYPFLAENQDKIEKILTFRIPYYVGPLARGKSEFAWLNRKS |
| | DEKIRPWNFDEMVDKETSAENFITRMTNYDQYLPDQKVLPKHSLLYEKFAVYNELTKVRYVTEQG |
| | KSFFFDANMKQEIFDGVFKVYRKVTKEKLMDFLGKEFDEFRIVDLMGLDKDNKSFNASLGTYHDL |
| | KKIVSKDFLDNPENEDILENVVLTLTLFEDREMIRKRLEKYKDVLTEEQRKKLERRHYTGWGRLSA |
| | KLINGIRDKVTRKTILDYLIDDGTSNRNFMQLIHDDTLSFVDEIRLAQGSGEAEDYRAEVQNLAGSP |
| | AIKKGILQSLKIVDELIEVMGYDPEHIVVEMARENQFTNQGRRNSQQRYKKIKDAIKNLDSNLNSKI |
| | LKEYPTNNQALQNDRLFLYYLQNGKDMYTGEALDINQLSNYDIDHIIPQAFIKDDSLDNRVLTSSK |
| | DNRGKSDNVPSLEVVEKMKAFWQQLLDSKLISERKYNNLTKAERERGGLNELDKVGFIKRQLVET |
| | RQITKHVAQILDARFNTEVDENNQRIRKVKIITLKSNLVSNFRKEFGLYKVREINDYHHAHDAYLN |
| | AVVAKAILKKYPKLEPEFVYGDYQKYDIKRYISRSKDPKEIEKATEKYFFYSNLLNFFKEEVHYAD |
| | GTIVKRENIEYSKDTGEIAWNKEKDFATVRKVLSCPQVNIVKKTEVQTGGFSKESILPKRNSDKLIA |
| | RKKDWDPKKYGGFDSPTVAYSVLVVAKVEKGKSKKLKSVKELLGITIMERSSFEKNPIDFLEAKG |
| | YKEVRKDLIVKLPKYSLFELENGRKRMLASAGELQKGNELALPSKYVNFLYLASHYEKLKGSPED |
| | NEQKQLFVEQHKHYLDEIIEQISEFSKRVILADANLDKVLSAYNKHRDKPIREQAENIIHLFTLTNLG |
| | APAAFKYFDTTIDRKRYTSTKEVLDATFIHQSITGLYETRIDLSQLGGD |
| 154 | MTKPYSIGLDIGTNSVGWAVITDSYKVPSKKMKVLGNTSKKYVKKNLLGALLFESGITAEGRRLK |
| | RTARRRYTRRRNRILYLQEIFSTEMAKIDESFFQRLDDSFLVPDDKRDSKYPIFGNLVEEKTYHDEFP |
| | TIYHLRKYLADSSKKADLRLVYLALAHMIKYRGHFLIEGDFNSRNNDIKKNFQDFLDSYNAIFESDL |
| | SLENSKQLEEIVKDKISKSAKKDRLLKLFPGEKNSGIFSEFLKLIVGNQADFRKYFNLDEKASLHFSK |
| | ESYDEDLETLLGHIGDDYSDVFLKAKKLYDAILLSGILTVTDNETEAPLSSAMIMRYKEHEEDLALL |
| | KAYIRNISLETYNEVFKDDTKNGYAGYIDGKTNQEDFYVYLKKLLTGLEGADYFLEKINREDLLRK |
| | QRTFDNGSIPYQIHLQEMRAIIDKQAKFYPFLAKNKERIEKILTFRIPYYVGPLARGNSDFAWSIRKR |
| | NEKITPWNFEDVIDKESSAEAFINRMTSFDLYLPEEKVLPKHSLLYETFTVYNELTKVRFIAESMRD |
| | YQFLDSKQKKDIVRLYFKGKRKVTDKDIIEYLHAIDGYDGIELKGIEKQFNSSLSTYHDLLKIIKDKE |
| | FLDDSSNEAIIEEIIHTLTIFEDREMIKQRLSKFENIFDKSVLKKLSRRHYTGWGKLSAKLINGIRDEK |
| | SGKTILDYLIDDGISNRNFMQLIHDDTLSFKKKIQKAQIIGDQDNIKQVVQSLPGSPAIKKGILQSIKI |
| | VDELVKVMGRNPESIVVEMARENQYTNQGKSNSQQRLKRLEESLKELGSKILKEKVPVKLSKIDN |
| | KALQNERLYLYYLQNGKDMYTGDDLDIDRLSNYDIDHIIPQAFLKDNSIDNKVLVSSASNRGKSDD |
| | VPSLDVVKKRKTFWHQLLKSKLISQRKFDNLTKAERGGLTEADKAGFIKRQLVETRQITKHVAQIL |
| | DARFNTKRDDNDKLIREVKIITLKSSLVSQFRKEFQLYKVREINDYHHAHDAYLNAVVATALLKKY |
| | PKLAPEFVYGEYNRYKSDSKKTKRVSVMSDLTVLFRDNVEYSNDTGEIAWNKEKDFATVRKVLSC |
| | PQVNIVKKTEVQTGGFSKESILPKRNSDKLIARKKDWDPKKYGGFDSPTVAYSVLVVAKVEKGKS |
| | KKLKSVKELLGITIMERSSFEKNPIDFLEAKGYKEVRKDLIVKLPKYSLFELENGRKRMLASAGELQ |
| | KGNELALPSKYVNFLYLASHYEKLKGSPEDNEQKQLFVEQHKHYLDEIIEQISEFSKRVILADANLD |
| | KVLSAYNKHRDKPIREQAENIIHLFTLTNLGAPAAFKYFDTTIDRKRYTSTKEVLDATFIHQSITGLY |
| | ETRIDLSQLGGD |
| 155 | MTKPYSIGLDIGTNSVGWAVITDSYKVPSKKMKVLGNTSKKYVKKNLLGALLFESGITAEGRRLK |
| | RTARRRYTRRRNRILYLQEIFSTEMAKIDESFFQRLDDSFLVPDDKRDSKYPIFGNLVKEKTYHDEF |
| | PTIYHLRKYLADSSKKADLRLVYLALAHMIKYRGHFLIEGDFNSRNNDIKKNFQDFLDSYNAIFESD |
| | LSLENSKQLEEIVKDKISKSAKKDRLLKLFPGEKNSGIFSEFLKLIVGNQADFRKYFNLDEKASLHES |
| | KESYDEDLETLLGHIGDDYSDVFLKAKKLYDAILLSGILTVTDNETEAPLSSAMIMRYKEHEEDLAL |
| | LKAYIRKISLETYNEVFKDDTKNGYAGYIDGKTNQEDFYVYLKKLLTGLEGADYFLEKINREDLLR |
| | KQRTFDNGSIPYQIHLQEMRAIIDKQAKFYPFLAKNKERIEKILTFRIPYYVGPLARGNSDFAWSIRK |
| | RNEKITPWNFEDVIDKESSAEAFINRMTSFDLYLPEEKVLPKHSLLYETFTVYNELTKVRFIAESMR |
| | DYQFLDSKQKKDIVRLYFKGKRKVTDKDIIEYLHAIDGYDGIELKGIEKQFNSSLSTYHDLLNIINDK |
| | EFLDDSSNEAIIEEIIHTLTIFEDREMIKQRLSKFENIFDKSVLKKLSRRHYTGWGKLSAKLINGIRDE |
| | KSGKTILDYLIDDGVSNRNFMQLIHDDTLSFKKKIQKAQIIGDQDNIKQVVQSLPGSPAIKKGILQSI |
| | KIVDELVKVMGRNPESIVVEMARENQYTNQGKSNSQQRLKRLEESLKELGSKILKENVPVKLSKID |
| | NNALQNDRLYLYYLQNGKDMYTGEELDIDNLSNYDIDHIIPQAFLKDDSIDNKVLVSSASNRGKSD |
| | DVPSLEVVRRRKNFWKQLLDSQLISQRKFDNLTKAERGGLTDDDKAGFIKRQLVETRQITKHVAQI |
| | LDARFNTKRDDNDKVIRNVKIITLKSSLVSQFRKEFGLYKVREINDYHHAHDAYLNAVVATAILKK |
| | YPKLEPEFVYGDYQKYDVRKYLSTKDNKDLGKATAKMFFYSNLMNFFKKEVRLSDGTVVVRPVI |
| | EYSKDTGEIAWNKEKDFATVRKVLSCPQVNIVKKTEVQTGGFSKESILPKRNSDKLIARKKDWDPK |
| | KYGGFDSPTVAYSVLVVAKVEKGKSKKLKSVKELLGITIMERSSFEKNPIDFLEAKGYKEVRKDLI |
| | VKLPKYSLFELENGRKRMLASAGELQKGNELALPSKYVNFLYLASHYEKLKGSPEDNEQKQLFVE |
| | QHKHYLDEIIEQISEFSKRVILADANLDKVLSAYNKHRDKPIREQAENIIHLFTLTNLGAPAAFKYFD |
| | TTIDRKRYTSTKEVLDATFIHQSITGLYETRIDLSQLGGD |
| 156 | MTKPYSIGLDIGTNSVGWAVITDSYKVPSKKMKVLGNTSKKYVKKNLLGALLFESGITAEGRRLK |
| | RTARRRYTRRRNRILYLQEIFSTEMAKIDESFFQRLDDSFLVPDDKRDSKYPIFGNLVEEKTYHDEFP |
| | TIYHLRKYLADSSKKADLRLVYLALAHMIKYRGHFLIEGDFNSRNNDIKKNFQDFLDSYNAIFESDL |
| | SLENSKQLEEIVKDKISKSAKKDRLLKLFPGEKNSGIFSEFLKLIVGNQADFRKYFNLDEKASLHFSK |
| | ESYDEDLETLLGHIGDDYSDVFLKAKKLYDAILLSGILTVTDNETEAPLSSAMIMRYKEHEEDLALL |
| | KAYIRKISLETYNEVFKDDTKNGYAGYIDGKTNQEDFYAYLKKLLTGLEGADYFLEKINREDLLRK |
| | QRTFDNGSIPHQIHLQEMHAILRKQEKFYPFLAKNKERIEKILTFRIPYYVGPLARGNSDFAWSIRKR |
| | NEKITPWNFEDVIDKESSAEAFINRMTSFDLYLPEEKVLPKHSLLYETFTVYNELTKVRFIAESMRD |
| | YQFLDSKQKKDIVRLYFKGKRKVTDKDIIDYLHAIDGYDGIELKGIEKQFNSSLSTYHDLLNIINDKE |
| | FLDDSSNEAIIEEIIHTLTIFEDREMIKQRLSKFENIFDKSVLKKLSRRHYTGWGKLSAKLINGIRDEK |
| | SGKTILDYLIDDGVSNRNFMQLIHDDTLSFKKKIQKAQIIGDQDNIKQVVQSLPGSPAIKKGILQSIKI |
| | VDELVKVMGRNPESIVVEMARENQYTNQGKSNSQQRLKRLEESLKELGSKILKENVPAKLSKIDN |
| | NALQNERLYLYYLQNGKDMYTGDDLDIDRLSNYDIDHIIPQAFLKDNSIDNKVLVSSASNRGKSDD |
| | VPSIDVVKKRKTFWHQLLKSQLISQRKFDNLTKAERGGLTEDDKAGFIKRQLVETRQITKHVAQIL |
| | DSRENTEVNDKGKKIRKVKIITLKSSLVSQFRKEFGLYKVREINDYHHAHDAYLNAVVATAILKKY |
| | PKLEPEFVYGDYQSYSLGHDKIVDVSKLIVKSDSSLGKATAKMFFYSNLMNFFKKKVRLSDGTVV |
| | VRPVVEYSNDTGEIAWNKEKDFATVRKVLSCPQVNIVKKTEVQTGGFSKESILPKRNSDKLIARKK |
| | DWDPKKYGGFDSPTVAYSVLVVAKVEKGKSKKLKSVKELLGITIMERSSFEKNPIDFLEAKGYKEV |
| | RKDLIVKLPKYSLFELENGRKRMLASAGELQKGNELALPSKYVNFLYLASHYEKLKGSPEDNEQK |
| | QLFVEQHKHYLDEIIEQISEFSKRVILADANLDKVLSAYNKHRDKPIREQAENIIHLFTLTNLGAPAA |
| | FKYFDTTIDRKRYTSTKEVLDATFIHQSITGLYETRIDLSQLGGD |
| 157 | MTKPYSIGLDIGTNSVGWAVITDSYKVPSKKMKVLGNTSKKYVKKNLLGALLFESGITAEGRRLK |
| | RTARRRYTRRRNRILYLQEIFSTEMAKIDEGFFQRLDDSFLVPDDKRDSKYPIFGNLVEEKVYHEEF |
| | PTIYHLRKYLADSSKKADLRLVYLALAHMIKYRGHFLIEGDFNSRNNDIKKNFQNFLDSYNAIFESD |
| | LSLENSKQLEEIVKDKISKSAKRERVLKLFPDEKSTGLFSEFLKLIVGNQADFKKHFDLEEKAPLQFS |
| | KDTYDEDLENLLGQIGDDFADLFLVAKKLYDAILLSGILTVTDPSTKAPLSASMIERYENHQKDLA |
| | ALKQFIQNNLQEKYDEVFSDQSKDGYAGYIDGKTTQEAFYKYIKNLLSKLEGADYFLDKIEREDFL |
| | RKQRTFDNGSIPHQIHLQEMNAIIRRQGEHYPFLKENKEKIEKILTFRIPYYVGPLARGNRDFAWLTR |
| | NSDQAIRPWNFEEIVDKASSAEDFINKMTNYDLYLPEEKVLPKHSLLYETFAVYNELTKVKFIAEGL |
| | RDYQFLDSGQKKQIVNQLFKEKRKVTEKDIIHYLHNVDGYDGIELKGIEKQFNASLSTYHDLLKIIK |
| | DKEFMDDPKNEEILENIVHTLTIFEDREMIKQRLAQYDSLFDEKVIKALTRRHYTGWGKLSAKLING |
| | IYDKQSRKTILDYLIDDDKINRNFMQLIHDDGLSFKEIIQKAQVFGKTDDVKQVVQELPGSPAIKKGI |
| | LQSIKIVDELVKVMGHAPESIVIEMARENQTTNQGKRNSQQRYKRIEDAIKNLSVDLNSKILKEYPT |
| | DNQALQNDRLFLYYLQNGKDMYTGEALDINQLSNYDIDHIIPQAFIKDDSLDNRVLTSSKDNRGKS |
| | DDVPSIEVVKARKGFWQQLLDSKLISERKENNLTKAERGGLDERDKVGFIRRQLVETRQITKHVAQ |
| | ILDARFNTEVNDKKQKIRTVKIVTLKSNLVSNFRKEFRLYKVREINDYHHAHDAYLNAVVAKAILK |
| | KYPKLEPEFVYGDYQKYDVKRYISRSKDPKDVEKATEKYFFYSNLLNFFKEEVHYADGTIIKRENIE |
| | YSKDTGEVAWNKEKDFATVRKVLSCPQVNIVKKTEVQTGGFSKESILPKRNSDKLIARKKDWDPK |
| | KYGGFDSPTVAYSVLVVAKVEKGKSKKLKSVKELLGITIMERSSFEKNPIDFLEAKGYKEVRKDLI |
| | VKLPKYSLFELENGRKRMLASAGELQKGNELALPSKYVNFLYLASHYEKLKGSPEDNEQKQLFVE |
| | QHKHYLDEIIEQISEFSKRVILADANLDKVLSAYNKHRDKPIREQAENIIHLFTLTNLGAPAAFKYFD |
| | TTIDRKRYTSTKEVLDATFIHQSITGLYETRIDLSQLGGD |
| 158 | MTKPYSIGLDIGTNSVGWAVITDSYKVPSKKMKVLGNTSKKYVKKNLLGALLFESGITAEGRRLK |
| | RTARRRYTRRRNRILYLQEIFSTEMAKIDESFFQRLDDSFLVPDDKRDSKYPIFGNLVEEKAYHEKF |
| | PTIYHLRKYLADSTKKADLRLVYLALAHMIKYRGHFLIEGDFNSRNNDIKKNFQNFLDSYNAIFESD |
| | LSLENSKQLEEIVKDKISKSAKRERVLKLFPDEKSTGLFSEFLKLIVGNQADFKKHFDLEEKAPLQFS |
| | KDTYDEDLENLLGQIGDGFADLFLVAKKLYDAILLSGILTVTDPSTKAPLSASMIERYENHQKDLAT |
| | LKQFIKSNLSEKYDEVFSDQSKDGYAGYIDGKTTQEAFYKYIKNLLSKLEGADYFLDKIEREDFLRK |
| | QRTFDNGSIPHQIHLQEMNAIIRRQGEHYPFLKENKEKIEKILTFRIPYYVGPLARGNRDFAWLTRNS |
| | DQAIRPWNFEEIVDQASSAEDFINKMTNYDLYLPEEKVLPKHSLLYETFAVYNELTKVKFIAEGMR |
| | DYQFLDSGQKKQIVNQLFKEKRKVTEKDIIQFLHNVDGYDGIELKGIEKQFNASLSTYHDLLKIIKD |
| | KDFMDDPKNEEILENIIHTLTIFEDREMIKQRLAQYDSLFDEKVIKALTRRHYTGWGKLSAKLINGI |
| | YDKQSKKTILDYLIDDGEINRNFMQLINDDGLSFKEIIQKAQVVGKTNDVKQVVQELPGSPAIKKGI |
| | LQSIKLVDELVKVMGHAPESIVIEMARENQTTARGKKNSQQRYKRIEDALKNLAPGLDSNILKEHP |
| | TDNVQLQNDRLFLYYLQNGKDMYTGEALDINQLSNYDIDHIVPQAFIKDDSLDNRVLTSSKDNRG |
| | KSDNVPSLEVVQKRKAFWQQLLDSKLISQRKFDNLTKAERGGLTEHDKAGFIKRQLVETRQITKH |
| | VAQILDARFNTEVDSKGKRIRKVKIITLKSNLVSNFRKEFGLYKVREINDYHHAHDAYLNAVVAKA |
| | ILKKYPKLEPEFVYGDYQKYDLKRYISRSKDPKDVEKATQKYFFYSNLLNFFKEEVHYADGTIIKRE |
| | NIEYSKDTGEIAWNKEKDFATVRKVLSCPQVNIVKKTEVQTGGFSKESILPKRNSDKLIARKKDWD |
| | PKKYGGFDSPTVAYSVLVVAKVEKGKSKKLKSVKELLGITIMERSSFEKNPIDFLEAKGYKEVRKD |
| | LIVKLPKYSLFELENGRKRMLASAGELQKGNELALPSKYVNFLYLASHYEKLKGSPEDNEQKQLFV |
| | EQHKHYLDEIIEQISEFSKRVILADANLDKVLSAYNKHRDKPIREQAENIIHLFTLTNLGAPAAFKYF |
| | DTTIDRKRYTSTKEVLDATFIHQSITGLYETRIDLSQLGGD |
| 159 | MTKPYSIGLDIGTNSVGWAVITDSYKVPSKKMKVLGNTSKKYVKKNLLGALLFESGITAEGRRLK |
| | RTARRRYTRRRNRILYLQEIFSTEMAEIDESFFQRLDDSFLVPDDKRDSKYPIFGNLVKEKTYHDEFP |
| | TIYHLRKYLADSSKKADLRLVYLALAHMIKYRGHFLIEGDFNSRNNDIKKNFQEFLDSYNAIFESDL |
| | SLENSKQLEEIVKDKISKSAKRERVLKLFPDEKSTGLFSEFLKLIVGNQADFKKHFDLEEKAPLQFSK |
| | DTYDEDLENLLGQIGDDFADLFLVAKKLYDAILLSGILTVTDPSTKAPLSASMIERYENHQKDLAAL |
| | KQFIQNNLQEKYDEVFSDQSKDGYAGYIDGKTTQEAFYKYIKNILTKVEGADYFLEKIKREDFLRK |
| | QRTFDNGTIPYQVHLQEMKAILHNQGEYYPFLKENREKIQQILTFRIPYYVGPLARGNRDFAWLTR |
| | NSDQAIRPWNFEEIVDQASSAEDFINKMTNYDLYLPEEKVLPKHSLLYETFAVYNELTKVKFIAEGL |
| | RDYQFLDSGQKKQIVNQLFKEKRKVTEKDIIHYLHNVDGYDGIELKGIEKQFNASLSTYHDLLKIIK |
| | DKEFMDDPKNEEILENIVHTLTIFEDREMIKQRLAQYDSLFDEKVIKALTRRHYTGWGKLSAKLING |
| | IYDKQSKKTILDYLIDDDKINRNFMQLINDDGLSFKDIIQKAQVVGKTDDVKQVVQELPGSPAIKKG |
| | ILQSIKLVDELVKVMGHAPESIVIEMARENQTTARGKKNSQQRYKRIEDALKNLAPGLDGNILKEY |
| | PTDNIQLQNDRLFLYYLQNGRDMYTGEPLDINQLSSYDIDHIVPQAFIKDDSLDNRVLTSSKDNRGK |
| | SDNVPSLEVVQKRKAFWQQLLDSKLISERKENNLTKAERGGLDERDKVGFIRRQLVETRQITKHVA |
| | QILDARFNAEVNDKKQRIRKVKIITLKSNLVSNFRKEFGLYKVREINDYHHAHDAYLNAVVAKAIL |
| | KKYPKLEPEFVYGDYQKYDLKRYISRSKDPKEIEKATQKYFFYSNLLNFFKEEVHYADGTIVKRENI |
| | EYSKDTGEIAWNKEKDFATVRKVLSCPQVNIVKKTEVQTGGFSKESILPKRNSDKLIARKKDWDPK |
| | KYGGFDSPTVAYSVLVVAKVEKGKSKKLKSVKELLGITIMERSSFEKNPIDFLEAKGYKEVRKDLI |
| | VKLPKYSLFELENGRKRMLASAGELQKGNELALPSKYVNFLYLASHYEKLKGSPEDNEQKQLFVE |
| | QHKHYLDEIIEQISEFSKRVILADANLDKVLSAYNKHRDKPIREQAENIIHLFTLTNLGAPAAFKYFD |
| | TTIDRKRYTSTKEVLDATFIHQSITGLYETRIDLSQLGGD |
| 160 | MTKPYSIGLDIGTNSVGWAVITDSYKVPSKKMKVLGNTSKKYVKKNLLGALLFESGITAEGRRLK |
| | RTARRRYTRRRNRILYLQEIFSTEMAKIDESFFQRLDDSFLVPDDKRDSKYPIFGNLVEEKTYHDEFP |
| | TIYHLRKYLADSQKKADLRLVYLALAHMIKYRGHFLIEGDENSKNNDIKKNFQEFLDSYNAIFESD |
| | LSLENSKQLEEIVKDKISKSAKRERVLKLFPDEKSTGLFSEFLKLIVGNQADFKKHFDLEEKAPLQFS |
| | KDTYDEDLENLLGQIGDDFADLFLVAKKLYDAILLSGILTVTDPSTKAPLSASMIERYENHQKDLA |
| | ALKQFIQNNLQEKYDEVFSDQSKNGYAGYIDGKTTQEEFYKYLKKILTGLEGADYFLEKIKREDLL |
| | RKQRTFDNGTIPYQVHLAEMRAIIHRQGEHYPFLKENKEKIEKILTFRIPYYVGPLARGNRDFAWLT |
| | RNSDQAIRPWNFEEVVDKASSAEDFINKMTNYDLYLPEEKVLPKHSLLYETFAVYNELTKVKFIAE |
| | GLRDYQFLDSGQKKQIVNQLFKEKRKVTEKDIIHYLHNVDGYDGIELKGIEKQFNASLSTYHDLLKI |
| | IKDKEFMDDAKNEEILENIVHTLTIFEDREMIKQRLAQYDSLFDEKVIKALTRRHYTGWGKLSAKLI |
| | NGISDKQTGKTILDYLIDDGKINRNFMQLIHDDGLSFKEIIQKAQVVGKTDDLKQVVQELPGSPAIK |
| | KGILQSIKIVDELVKVMGHAPESIVIEMARENQTTNQGRRNSQQRYKKIEDAIKNLDSNLNSKILKE |
| | YPTDNQALQNERLFLYYLQNGKDMYTGEALDINQLSNYDIDHIVPQAFIKDDSLDNRVLTSSKDNR |
| | GKSDNVPSIEIVQKRKAFWQQLLDSKLISERKENNLTKAERGGLDERDKVGFIKRQLVETRQITKH |
| | VAQILDARFNTEVDENNQKIRTVKIITLKSNLVSNFRKEFGLYKVREINDYHHAHDAYLNAVLAKA |
| | ILKKYPKLEPEFVYGDYQKYDLKRYISRSKDPKDIEKATEKYFFYSNLLNFFKEEVHYADGTIIKRG |
| | NVEYSKDTGEIAWNKEKDFATVRKVLSCPQVNIVKKTEVQTGGFSKESILPKRNSDKLIARKKDW |
| | DPKKYGGFDSPTVAYSVLVVAKVEKGKSKKLKSVKELLGITIMERSSFEKNPIDFLEAKGYKEVRK |
| | DLIVKLPKYSLFELENGRKRMLASAGELQKGNELALPSKYVNFLYLASHYEKLKGSPEDNEQKQLF |
| | VEQHKHYLDEIIEQISEFSKRVILADANLDKVLSAYNKHRDKPIREQAENIIHLFTLTNLGAPAAFKY |
| | FDTTIDRKRYTSTKEVLDATFIHQSITGLYETRIDLSQLGGD |
| 161 | MTKPYSIGLDIGTNSVGWAVITDSYKVPSKKMKVLGNTSKKYVKKNLLGALLFESGITAEGRRLK |
| | RTARRRYTRRRNRILYLQEIFATEMAKIDESFFQRLDDSFLVPDDKRDSKYPIFGNLVEEKTYHDEF |
| | PTIYHLRKYLADSQKKADLRLVYLALAHMIKYRGHFLIEGDFNSRNNDIKKNFQEFLDSYNAIFESD |
| | LSLENSKQLEEIVKDKISKSAKRERVLKLFPDEKSTGLFSEFLKLIVGNQADFKKHFDLEEKAPLQFS |
| | KDTYDEDLENLLGQIGDDFADLFLVAKKLYDAILLSGILTVTDPSTKAPLSASMIERYENHQKDLA |
| | ALKQFIQNNLQEKYDEVFSDQSKDGYAGYIDGKTTQEAFYKYIKNLLSKFEGADYFLDKIEREDFL |
| | RKQRTFDNGSIPHQIHLQEMNAILRRQGEHYPFLKENKEKIEKILTFRIPYYVGPLARGNRDFAWLT |
| | RNSDQAIRPWNFEEVVDKASSAEDFINKMTNYDLYLPEEKVLPKHSLLYETFAVYNELTKVKFIAE |
| | GLRDYQFLDSGQKKQIVNQLFKEKRKVTEKDIIHFLHNVDGYDGIELKGIEKQFNASLSTYHDLLKI |
| | IKDKEFMDDPKNEEILENIVHTLTIFEDREMIKQRLAQYDSLFDEKVIKALTRRHYTGWGKLSAKLI |
| | NGIRDKQTNKTILDYLIDDDKINRNFMQLIHDDGLSFKEIIQKAQVVGDTDDLRQVVQNLPGSPAIK |
| | KGILQSIKIVDELVKVMGHAPEQIVIEMARENQTTNQGRRNSQQRYKLIEDGVKNLASDLNGNILK |
| | EYPTDNQALQNDRLFLYYLQNGKDMYTGEELDINQLSNYDIDHIVPQAFIKDDSLDNRVLTSSKDN |
| | RGKSDNVPSIEVVQKRKAFWQQLLDSKLISERKENNLTKAERGGLDERDKVGFIRRQLVETRQITK |
| | HVAQILDARFNTEVDENGKRIRKVKIITLKSNLVSNFRKEFGLYKVREINDYHHAHDAYLNAVVAK |
| | AILKKYPKLEPEFVYGDYQKYDISRYISRSKVSDSIDKATAKYFFYSNLLNFFKEKVRYSNGTIIVRP |
| | VIEYSKDTGEIAWNKEKDFATVRKVLSCPQVNIVKKTEVQTGGFSKESILPKRNSDKLIARKKDWD |
| | PKKYGGFDSPTVAYSVLVVAKVEKGKSKKLKSVKELLGITIMERSSFEKNPIDFLEAKGYKEVRKD |
| | LIVKLPKYSLFELENGRKRMLASAGELQKGNELALPSKYVNFLYLASHYEKLKGSPEDNEQKQLFV |
| | EQHKHYLDEIIEQISEFSKRVILADANLDKVLSAYNKHRDKPIREQAENIIHLFTLTNLGAPAAFKYF |
| | DTTIDRKRYTSTKEVLDATFIHQSITGLYETRIDLSQLGGD |
| 162 | MTKPYSIGLDIGTNSVGWAVITDSYKVPSKKMKVLGNTSKKYVKKNLLGALLFESGITAEGRRLK |
| | RTARRRYTRRRNRILYLQEIFSTEMSEIDESFFQRLDDSFLVPDDKRDSKYPIFGNLVEEKTYHDEFP |
| | TIYHLRKYLADSQKKADLRLVYLALAHMIKYRGHFLIEGDFNSRNNDIKKNFQDFLDSYNAIFESD |
| | LSLENSEQLEEIVKDKISKSAKRERVLKLFPDEKSTGLFSEFLKLIVGNQADFKKHFDLEEKAPLQFS |
| | KDTYDEDLENLLGQIGDDFADLFLVAKKLYDAILLSGILTVTDPSTKAPLSASMIERYENHQKDLSA |
| | LKQFIKRNLSEKYDEVFSDESKDGYAGYINGKTTQEAFYKYIKNLLSKLEGADYFLEKIEREDFLRK |
| | QRTFDNGSIPHQIHLQEMNAIIRRQGEHYPFLKENKEKIQQILTFRIPYYVGPLARGNRDFAWLTRNS |
| | DQAIRPWNFEEIVDKASSAEDFINKMTNYDLYLPEEKVLPKHSLLYETFAVYNELTKVKFIAEGLR |
| | DYQFLDSGQKKQIVNQLFKEKRKVTEKDIIHYLHNVDGYDGIELKGIEKQFNASLSTYHDLLKIIKD |
| | KEFMDDPKNEEILENIVHTLTIFEDREMIKQRLAQYDSLFDEKVIKALTRRHYTGWGKLSAKLINGI |
| | CDKKTGNTILDYLIDDDKINRNFMQLINDDGLSFKEIIQKAQVVGKTDDVKQVVQELPGSPAIKKGI |
| | LQSIKIVDELVKVMGHEPESIVIEMARENQTTARGKKNSQQRYKRIEDALKNLAPGLDSNILKEHPT |
| | DNIQLQNDRLFLYYLQNGKDMYTGEELDIHHLSDYDIDHIVPQAFIKDDSIDNRVLTSSKDNRGKS |
| | DNVPSIEVVQKRKAFWQQLLDSKLISEHKFNNLTKAERGGLDERDKVGFIRRQLVETRQITKHVAQ |
| | ILDARFNTEVNEKGQKIRKVKIVTLKSNLVSNFRKEFELYKVREINDYHHAHDAYLNAVVAKAILK |
| | KYPKLEPEFVYGDYQKYDVKRYISRSKDPRDLHKATEKYFFYSNLLNFFKEEVHYADGTIVFRRNV |
| | EYSNDTGEIAWNKEKDFATVRKVLSCPQVNIVKKTEVQTGGFSKESILPKRNSDKLIARKKDWDPK |
| | KYGGFDSPTVAYSVLVVAKVEKGKSKKLKSVKELLGITIMERSSFEKNPIDFLEAKGYKEVRKDLI |
| | VKLPKYSLFELENGRKRMLASAGELQKGNELALPSKYVNFLYLASHYEKLKGSPEDNEQKQLFVE |
| | QHKHYLDEIIEQISEFSKRVILADANLDKVLSAYNKHRDKPIREQAENIIHLFTLTNLGAPAAFKYFD |
| | TTIDRKRYTSTKEVLDATFIHQSITGLYETRIDLSQLGGD |
| 163 | MTKPYSIGLDIGTNSVGWAVITDSYKVPSKKMKVLGNTSKKYVKKNLLGALLFESGITAEGRRLK |
| | RTARRRYTRRRNRILYLQEIFSTEMSKIDESFFQRLDDSFLVPDDKRDSKYPIFGNLVEEKTYHDEFP |
| | TIYHLRKYLADSSKKADLRLVYLALAHMIKYRGHFLIEGDFNSRNNDIKKNFQQFLDSYNSLYESD |
| | FSLENNAQVEEIVKDKISKSAKRERVLKLFPDEKSTGLFSEFLKLIVGNQADFKKHFDLEEKAPLQF |
| | SKDTYDEDLENLLGQIGDDFADLFLVAKKLYDAILLSGILTVTDPSTKAPLSASMIERYENHQKDLA |
| | ALKQFIKNNIPEKYDEVFADQSKDGYAGYIDGKTTQEAFYKYVKEKLNNLKGADYFLDKIEREDF |
| | LRKQRTFDNGSIPHQIHLQEMNAIIRRQGEHYPFLKENKEKIEKILTFRIPYYVGPLARGNRDFAWLT |
| | RNSDQAIRPWNFEEIVDQASSAEEFINKMTNYDLYLPEEKVLPKHSLLYETFAVYNELTKVKFIAEG |
| | LRDYQFLDSGQKKQIVNQLFKEKRKVTEKDIIQFLHNVDGYDGIELKGIEKQFNASLSTYHDLLKII |
| | KDKEFMDDPKNEEILENIVHTLTIFEDREMIKQRLAQYDSLFDEKVIKALTRRHYTGWGKLSAKLIN |
| | GICDKKTGKTILDYLIDDDKINRNFMQLINDDGLSFKDIIQKAQVVGKTDDVKQVVQELPGSPAIKK |
| | GILQSIKIVDELVKVMGHAPESIVIEMARENQTTARGKKNSQQRYKRIEDALKNLAPGLDSNILKEH |
| | PTDNTALQNDRLFLYYLQNGRDMYTGNPLDINQLSSYDIDHIVPQAFIKDDSLDNRVLTSSKDNRG |
| | KSDNVPSLEVVQKRKAFWQQLLDSKLISERKENNLTKAERGGLDERDKVGFIKRQLVETRQITKHV |
| | AQILDARFNTEVDENNQKIRKVKIVTLKSNLVSNFRKEFGLYKVREINDYHHAHDAYLNAVVAKA |
| | ILKKYPKLEPEFVYGDYQKYDIKRYISRSKDPKEIEKATEKYFFYSNLLNFFKEEVHYADGTIVKRE |
| | NIEYSKDTGEIAWNKEKDFATVRKVLSCPQVNIVKKTEVQTGGFSKESILPKRNSDKLIARKKDWD |
| | PKKYGGFDSPTVAYSVLVVAKVEKGKSKKLKSVKELLGITIMERSSFEKNPIDFLEAKGYKEVRKD |
| | LIVKLPKYSLFELENGRKRMLASAGELQKGNELALPSKYVNFLYLASHYEKLKGSPEDNEQKQLFV |
| | EQHKHYLDEIIEQISEFSKRVILADANLDKVLSAYNKHRDKPIREQAENIIHLFTLTNLGAPAAFKYF |
| | DTTIDRKRYTSTKEVLDATFIHQSITGLYETRIDLSQLGGD |
| 164 | MTKPYSIGLDIGTNSVGWAVITDSYKVPSKKMKVLGNTSKKYVKKNLLGALLFESGITAEGRRLK |
| | RTARRRYTRRRNRILYLQEIFSTEMAKIDESFFQRLDDSFLVPDDKRDSKYPIFGNLVEEKTYHDEFP |
| | TIYHLRKYLADSQKKADLRLVYLALAHMIKYRGHFLIEGDFNSRNNDIKKNFQDFLDSYNAIFESD |
| | LSLENSKQLEEIVKDKISKSAKRERVLKLFPDEKSTGLFSEFLKLIVGNQADFKKHFNLEEKAPLHFS |
| | KDTYDEDLENLLGQIGDDFADLFLVAKKLYDAILLSGILTVTDPSTKAPLSASMIERYENHQKDLA |
| | ALKQFIQNNLQEKYDEVFSDQSKDGYAGYIDGKTTQEAFYKYIKNLLSKFEGADYFLEKIEREDFL |
| | RKQRTFDNGSIPYQIHLQEMRAILSRQGEYYPFLKDNKEKIEKILTFRIPYYVGPLARGNRDFAWLT |
| | RNSDQAIRPWNFEEIVDQASSAEDFINKMTNYDLYLPEEKVLPKHSLLYETFAVYNELTKVKFIAEG |
| | LRDYQFLDSGQKKQIVNQLFKDKRKVTEKDIIHYLHNVDGYDGIELKGIEKQFNASLSTYHDLLKII |
| | KDKEFMDDPKNEEILENIVHTLTIFEDREMIKQRLAQYDSLFDEKVIKALTRRHYTGWGKLSAKLIN |
| | GIRDKKSGKTILDYLIDDDKINRNFMQLINDDGLSFKDIIQKAQVVGKTDDVKQVVQELPGSPAIKK |
| | GILQSIKIVDELVKVMGHAPESIVIEMARENQTTARGKKNSQQRYKRIEDALKNLAPGLDSNILKEH |
| | PTDNIQLQNDRLFLYYLQNGKDMYTGNPLDINQLSSYDIDHIVPQAFIKDDSLDNRVLTSSKDNRG |
| | KSDNVPSLEVVQKRKAFWQQLLDSKLISERKYNNLTKAERERDGLNELDKVGFIKRQLVETRQITK |
| | HVAQILDARFNTEVNEKNQKIRKVKIITLKSNLVSNFRKEFGLYKVREINDYHHAHDAYLNAVVA |
| | KAILKKYPKLEPEFVYGDYQKYDLKRYISRSKDPKDIEKATEKYFFYSNLLNFFKEEVHYADGTIIK |
| | RENIEYSNDTGEIAWNKEKDFATVRKVLSCPQVNIVKKTEVQTGGFSKESILPKRNSDKLIARKKD |
| | WDPKKYGGFDSPTVAYSVLVVAKVEKGKSKKLKSVKELLGITIMERSSFEKNPIDFLEAKGYKEVR |
| | KDLIVKLPKYSLFELENGRKRMLASAGELQKGNELALPSKYVNFLYLASHYEKLKGSPEDNEQKQ |
| | LFVEQHKHYLDEIIEQISEFSKRVILADANLDKVLSAYNKHRDKPIREQAENIIHLFTLTNLGAPAAF |
| | KYFDTTIDRKRYTSTKEVLDATFIHQSITGLYETRIDLSQLGGD |
| 165 | MTKPYSIGLDIGTNSVGWAVITDSYKVPSKKMKVLGNTSKKYVKKNLLGALLFESGITAEGRRLK |
| | RTARRRYTRRRNRILYLQEIFATEMAKIDESFFQRLDDSFLVPDDKRDSKYPIFGNLVKEKTYHDEF |
| | PTIYHLRKYLADSSKKADLRLVYLALAHMIKYRGHFLIEGDFNSRNNDIKKNFQEFLDSYNAIFESD |
| | LSLENSKQLEEIVKDKISKSAKRERVLKLFPDEKSTGLFSEFLKLIVGNQADFKKHFDLEEKAPLQFS |
| | KDTYDEDLENLLGQIGDDFADLFLVAKKLYDAILLSGILTVTDPSTKAPLSASMIERYENHQKDLAT |
| | LKQFIKNNLSEKYDEVFSDQSKDGYAGYIDGKTTQEAFYKYIKNTLSKIEGADYFLDKIEREDFLRK |
| | QRTFDNGSIPHQIHLQEMHAIIHRQGEYYPFLQENKEKIEKILTFRIPYYVGPLARGNRDFAWLTRNS |
| | DQAIRPWNFEEIVDKASSAEDFINKMTNYDLYLPEEKVLPKHSLLYETFAVYNELTKVKFIAEGLR |
| | DYQFLDSGQKKQIVNQLFKEKRKVTEKDIIQFLHNVDGYDGIELKGIEKQFNASLSTYHDLLKIIKD |
| | KEFMDDPKNEEILENIVHTLTIFEDREMIKQRLAQYDTLFDEKVIKALTRRHYTGWGKLSAKLINGI |
| | RDKQSQKTILDYLIDDDKINRNFMQLINDDGLSFKDIIQKAQVVGKTDDVKQVVQELPGSPAIKKGI |
| | LQSIKIVDELVKVMGHAPESIVIEMARENQTTARGKKNSQQRYKRIEDALKNLAPGLDSNILKENPT |
| | DNIQLQNDRLFLYYLQNGKDMYTGEELDINQLSNYDIDHIVPQAFIKDDSLDNRVLTSSKDNRGKS |
| | DNVPSIEVVQKRKAFWQQLLDSKLISERKENNLTKAERGGLDERDKVGFIKRQLVETRQITKHVAQ |
| | ILDARFNTEVNDKNQKIRTVKIITLKSNLVSNFRKEFRLYKVREINDYHHAHDAYLNAVVAKAILK |
| | KYPKLEPEFVYGDYHHYDLKRQISKSKISKSIDKATQKYFFYSNLLNFFKEEVHYADGTIIKRENIEY |
| | SKDTGEIAWNKEKDFATVRKVLSCPQVNIVKKTEVQTGGFSKESILPKRNSDKLIARKKDWDPKK |
| | YGGFDSPTVAYSVLVVAKVEKGKSKKLKSVKELLGITIMERSSFEKNPIDFLEAKGYKEVRKDLIV |
| | KLPKYSLFELENGRKRMLASAGELQKGNELALPSKYVNFLYLASHYEKLKGSPEDNEQKQLFVEQ |
| | HKHYLDEIIEQISEFSKRVILADANLDKVLSAYNKHRDKPIREQAENIIHLFTLTNLGAPAAFKYFDT |
| | TIDRKRYTSTKEVLDATFIHQSITGLYETRIDLSQLGGD |
| 166 | MTKPYSIGLDIGTNSVGWAVITDSYKVPSKKMKVLGNTSKKYVKKNLLGALLFESGITAEGRRLK |
| | RTARRRYTRRRNRILYLQEIFSTEMAKIDESFFQRLDDSFLVPDDKRDSKYPIFGNLVEEKTYHDEFP |
| | TIYHLRKYLADSSKKADLRLVYLALAHMIKYRGHFLIEGDFNSRNNDIKKNFQNFLDSYNAIFESDL |
| | SLENSKQLEEIVKDKISKSAKRERVLKLFPDEKSTGLFSEFLKLIVGNQADFKKHFDLEEKAPLQFSK |
| | DTYDEDLENLLGQIGDDFADLFLVAKKLYDAILLSGILTVTDPSTKAPLSASMIERYENHQKDLAAL |
| | KQFIQNNLQEKYDEVFSDQSKDGYAGYIDGKTTQEAFYKYIKNLLAKFEGADYFLEKIEREDFLRK |
| | QRTFDNGSIPYQIHLQEMQAIIHRQGEYYPFLKENKEKIEKILTFRIPYYVGPLARGNRDFAWLTRNS |
| | DQAIRPWNFEEIVDQASSAEEFINKMTNYDLYLPEEKVLPKHSLLYETFAVYNELTKVKFIAEGLRD |
| | YQFLDSGQKKQIVNQLFKEKRKVTEKDIIHYLHNVDGYDGIELKGIEKQFNASLSTYHDLLKIIKDK |
| | AFMDDAKNEAILENIIHTLTIFEDREMIKQRLAQYDSLFDEKVIKALTRRHYTGWGKLSAKLINGIC |
| | DKQTGDTILDYLIDDDKINRNFMQLINDDGLSFKEIIQKAQVVGKTDDVKQVVQELPGSPAIKKGIL |
| | QSIKIVDELVKVMGYEPEQIVIEMARENQTTNQGRRNSQQRYKLLEDGVKNLAKDLDGNILKEYPT |
| | DNQALQNDRLFLYYLQNGKDMYTGEPLDINQLSSYDIDHIIPQAFIKDDSLDNRVLTSSKDNRGKS |
| | DNVPSIEVVQKRKAFWQQLLDSKLISERKENNLTKAERGGLDERDKVGFIKRQLVETRQITKHVAQ |
| | ILDARFNVEVNDKKQKIRTVKIITLKSNLVSNFRKEFGLYKVREINDYHHAHDAYLNAVVAKAILK |
| | KYPKLEPEFVYGDYQKYDLKRYISRSKDPKDVEKATQKYFFYSNLLNFFKEEVHYADGTIIKRENIE |
| | YSKDTGEIAWNKEKDFATVRKVLSCPQVNIVKKTEVQTGGFSKESILPKRNSDKLIARKKDWDPK |
| | KYGGFDSPTVAYSVLVVAKVEKGKSKKLKSVKELLGITIMERSSFEKNPIDFLEAKGYKEVRKDLI |
| | VKLPKYSLFELENGRKRMLASAGELQKGNELALPSKYVNFLYLASHYEKLKGSPEDNEQKQLFVE |
| | QHKHYLDEIIEQISEFSKRVILADANLDKVLSAYNKHRDKPIREQAENIIHLFTLTNLGAPAAFKYFD |
| | TTIDRKRYTSTKEVLDATFIHQSITGLYETRIDLSQLGGD |
| 167 | MTKPYSIGLDIGTNSVGWAVITDNYKVPSKKMKVLGNTSKKYVKKNLLGALLFESGITAEGRRLK |
| | RTARRRYTRRRNRILYLQEIFATEMNKIDESFFQRLDDSFLVPDDKRDSKYPIFGNLVEEKTYHDEF |
| | PTIYHLRKYLADSSKKADLRLVYLALAHMIKYRGHFLIEGDFNSRNNDIKKNFQDFLDSYNAIFESD |
| | LSLENSKQLEEIVKDKISKSAKRERVLKLFPDEKSTGLFSEFLKLIVGNQADFKKHFDLEEKAPLQFS |
| | KDTYDEDLENLLGQIGDDFADLFLVAKKLYDAILLSGILTVTDPSTKAPLSASMIERYENHQKDLA |
| | ALKQFIQNNLQEKYDEVFSDQSKDGYAGYIDGKTTQEAFYKYIKNLLSKFEGADYFLEKIEREDFL |
| | RKQRTFDNGSIPHQIHLQEMNAIIRRQGEYYPFLQENKEKIEKILTFRIPYYVGPLARGNRDFAWLTR |
| | NSDQAIRPWNFEEIVDQASSAEDFINKMTNYDLYLPEEKVLPKHSLLYETFAVYNELTKVKFIAEGL |
| | RDYQFLDSGQKKQIVNQLFKEKRKVTEKDIIHFLHNVDGYDGIELKGIEKQFNASLSTYHDLLKIIK |
| | DKEFMDDPKNEEILENIVHTLTIFEDREMIKQRLAQYDSLFDEKVIKALTRRHYTGWGKLSAKLING |
| | IRDKQSQKTILDYLIDDDQINRNFMQLINDDGLSFKEIIQKAQVVGKTDDLRQVVQNLPGSPAIKKG |
| | ILQSIKLVDELVKVMGKDPESIVIEMARENQTTARGKKNSQQRYKRIEDALKNLAPGLDSNILKEHP |
| | TDNIQLQNDRLYLYYLQNGKDMYTGNPLDINQLSNYDIDHIVPQAFIKDDSIDNRVLTSSKDNRGK |
| | SDNVPSIEIVQKRKAFWQQLLDSKLISERKENNLTKAERGGLDEKDKVGFIRRQLVETRQITKHVA |
| | QILDARFNTELDENGKRIRKVKIVTLKSNLVSNFRKEFGLYKVREINDYHHAHDAYLNAVVAKAIL |
| | KKYPKLEPEFVYGDYQKYDVKRYISRSKNPKEIDKATEKYFFYSNLLNFFKEEVHYADGTIVKREN |
| | IEYSKDTGEIAWNKEKDFATVRKVLSCPQVNIVKKTEVQTGGFSKESILPKRNSDKLIARKKDWDP |
| | KKYGGFDSPTVAYSVLVVAKVEKGKSKKLKSVKELLGITIMERSSFEKNPIDFLEAKGYKEVRKDL |
| | IVKLPKYSLFELENGRKRMLASAGELQKGNELALPSKYVNFLYLASHYEKLKGSPEDNEQKQLFVE |
| | QHKHYLDEIIEQISEFSKRVILADANLDKVLSAYNKHRDKPIREQAENIIHLFTLTNLGAPAAFKYFD |
| | TTIDRKRYTSTKEVLDATFIHQSITGLYETRIDLSQLGGD |
| 168 | MTKPYSIGLDIGTNSVGWAVITDSYKVPSKKMKVLGNTSKKYVKKNLLGALLFESGITAEGRRLK |
| | RTARRRYTRRRNRILYLQEIFSTEMAKIDESFFQRLDDSFLVPDDKRDSKYPIFGNLVKEKTYHDEF |
| | PTIYHLRKYLADSSKKADLRLVYLALAHMIKYRGHFLIEGDFNSRNNDIKKNFQDFLDSYNAIFESD |
| | LSLENSKQLEEIVKDKISKSAKRERVLKLFPDEKSTGLFSEFLKLIVGNQADFKKHFDLEEKAPLQFS |
| | KDTYDEDLENLLGQIGDDFADLFLVAKKLYDAILLSGILTVTDPSTKAPLSASMIERYENHQKDLA |
| | ALKQFIKGNLSEKYDEVFSDQSKDGYAGYIDGKTTQEAFYKYIKNLLSKLEGADYFLDKIEREDFL |
| | RKQRTFDNGSIPHQIHLQEMNAIIRRQGEHYPFLKENKEKIEKILTFRIPYYVGPLARGNRDFAWLTR |
| | NSDQAIRPWNFEEIVDQASSAEDFINKMTNYDLYLPEEKVLPKHSLLYETFAVYNELTKVKFIAEGL |
| | RDYQFLDSGQKKQIVNQLFKEKRKVTEKDIIHFLHNVDGYDGIELKGIEKQFNASLSTYHDLLKIIK |
| | DKEFMDDPKNEEILENIVHTLTIFEDREMIKQRLAQYDSLFDEKVIKALTRRHYTGWGKLSAKLING |
| | ICDKQTGDTILDFLIDDDKINRNFMQLINDDGLSFKEIIQKAQVVGKTDDLKQVVQELPGSPAIKKGI |
| | LQSIKIVDELVKVMGHAPEQIVIEMARENQTTNQGRRNSRQRYKLIEDGVKNLASDLNGNILKEYP |
| | TDNQALQNDRLFLYYLQNGKDMYTGEELDINQLSNYDIDHIVPQAFIKDDSLDNRVLTSSKDNRG |
| | KSDNVPSLEIVQKRKGFWQQLLDSKLISERKENNLTKAERGGLDERDKVGFIKRQLVETRQITKHV |
| | AQILDSRFNTEVDEKGKKIRDVKIITLKSNLVSNFRKEFGLYKVREINDYHHAHDAYLNAVVAKAI |
| | LKKYPKLEPEFVYGDYQKYDIKRYISRSKDPKDVEKATEKYFFYSNLLNFFKEEVHYADGTIVKRE |
| | NIEYSKDTGEIAWNKEKDFATVRKVLSCPQVNIVKKTEVQTGGFSKESILPKRNSDKLIARKKDWD |
| | PKKYGGFDSPTVAYSVLVVAKVEKGKSKKLKSVKELLGITIMERSSFEKNPIDFLEAKGYKEVRKD |
| | LIVKLPKYSLFELENGRKRMLASAGELQKGNELALPSKYVNFLYLASHYEKLKGSPEDNEQKQLFV |
| | EQHKHYLDEIIEQISEFSKRVILADANLDKVLSAYNKHRDKPIREQAENIIHLFTLTNLGAPAAFKYF |
| | DTTIDRKRYTSTKEVLDATFIHQSITGLYETRIDLSQLGGD |
| 169 | MTKPYSIGLDIGTNSVGWAVITDSYKVPSKKMKVLGNTSKKYVKKNLLGALLFESGITAEGRRLK |
| | RTARRRYTRRRNRILYLQEIFSTEMAKIDESFFQRLDDSFLVPDDKRDSKYPIFGNLVEEKTYHDEFP |
| | TIYHLRKYLADSSKKADLRLVYLALAHMIKYRGHFLIEGDFNSRNNDIKKNFQNFLDSYNAIFESDL |
| | SLENNAQVEEIVKDKISKSAKRERVLKLFPDEKSTGLFSEFLKLIVGNQADFKKHFDLEEKAPLQFS |
| | KDTYDEDLENLLGQIGDDFADLFLVAKKLYDAILLSGILTVTDPSTKAPLSASMIERYENHQKDLA |
| | ALKQFIKNNLPEKYDEVFSDESKDGYAGYINGKTTQEAFYKYIKNLLSKLEGADYFLDKIEREDFL |
| | RKQRTFDNGSIPHQIHLQEMNAIIRRQGEHYPFLKENKEKIEKILTFRIPYYVGPLARGNRDFAWLTR |
| | NSDQAIRPWNFEEVVDKASSAEDFINKMTNYDLYLPEEKVLPKHSLLYETFAVYNELTKVKFIAEG |
| | LRDYQFLDSGQKKQIVNQLFKDKRKVTEKDIIHYLHNVDGYDGIELKGIEKQFNASLSTYHDLLKII |
| | KDKEFMDDPKNEEILENIVHTLTIFEDREMIKQRLAQYDSLFDEKVIKALTRRHYTGWGKLSAKLIN |
| | GISDKQTGNTILDYLIDDDKINRNFMQLIHDDGLSFKEIIQKAQVVGESDNLKQVVQNLPGSPAIKK |
| | GILQSIKIVDELVKVMGKDPEHIVVEMARENQFTNQGRRNSQQRYKKIMDAIKNLDSNLNSKILKE |
| | YPTNNQALQNDRLFLYYLQNGKDMYTGEPLDINQLSNYDIDHIIPQAFIKDDSLDNRVLTSSKDNR |
| | GKSDNVPSLEVVEKMKAFWQQLLDSKLISERKYNNLTKAERERGGLNELDKVGFIKRQLVETRQI |
| | TKHVAQILDARFNTEVNEKGKKIRKVKIITLKSNLVSNFRKEFGLYKVREINDYHHAHDAYLNAVV |
| | AKAILKKYPKLEPEFVYGDYQKYDIKRYISRSKDPKDIEKATEKYFFYSNLLNFFKEEVHYADGTIV |
| | KRENIEYSKDTGEIAWNKEKDFATVRKVLSCPQVNIVKKTEVQTGGFSKESILPKRNSDKLIARKK |
| | DWDPKKYGGFDSPTVAYSVLVVAKVEKGKSKKLKSVKELLGITIMERSSFEKNPIDFLEAKGYKEV |
| | RKDLIVKLPKYSLFELENGRKRMLASAGELQKGNELALPSKYVNFLYLASHYEKLKGSPEDNEQK |
| | QLFVEQHKHYLDEIIEQISEFSKRVILADANLDKVLSAYNKHRDKPIREQAENIIHLFTLTNLGAPAA |
| | FKYFDTTIDRKRYTSTKEVLDATFIHQSITGLYETRIDLSQLGGD |
| 170 | MTKPYSIGLDIGTNSVGWAVITDSYKVPSKKMKVLGNTSKKYVKKNLLGALLFESGITAEGRRLK |
| | RTARRRYTRRRNRILYLQEIFSTEMAKIDESFFQRLDDSFLVPDDKRDSKYPIFGNLVEEKTYHDEFP |
| | TIYHLRKYLADSSKKADLRLVYLALAHMIKYRGHFLIEGDFNSRNNDIKKNFQDFLDSYNAIFESDL |
| | SLENSKQLEEIVKDKISKSAKKDRLLKLFPGEKNSGIFSEFLKLIVGNQADFRKYFNLDEKASLHFSK |
| | ESYDEDLETLLGHIGDDYSDVFLKAKKLYDAILLSGILTVTDNETEAPLSSAMIMRYKEHEEDLALL |
| | KAYIRKISLETYNEVFKDDTKNGYAGYIDGKTNQEDFYVYLKKLLTGLEGADYFLEKINREDLLRK |
| | QRTFDNGKIPYQIHLQEMRAIIDKQAKFYPFLAKNKEKIEKILTFRIPYYVGPLARGNSDFAWSIRKR |
| | NEKITPWNFEDVIDKESSAEAFINRMTSFDLYLPEEKVLPKHSLLYETFTVYNELTKVRFIAESMRD |
| | YQFLDSKQKKDIVRLYFKGKRKVTDKDIIDYLHAIDGYDGIELKGIEKQFNSSLATYHDLLNIINDK |
| | EFLDDSSNEAIIEEIIHTLTIFEDREMIKQRLSKFENIFDKSVLKKLSRRHYTGWGKLSAKLINGIRDE |
| | KSGKTILDYLIDDGVSNRNFMQLIHDDTLSFKKKIQKAQIIGDQDNIKQVVQSLPGSPAIKKGILQSI |
| | KIVDELVKVMGREPESIVVEMARENQYTNQGKSNSQQRLKRLEESLKDLGSKILKENVPAKLSKID |
| | NNALQNERLYLYYLQNGKDMYTGQDLDIDRLSNYDIDHIIPQAFLKDNSIDNKVLVSSASNRGKSD |
| | DVPSLEVVRQRKTFWHQLLKSKLISQRKFDNLTKAERGGLTEDDKAGFIKRQLVETRQITKHVAQI |
| | LDSRFNTERDDNDKVIRNVKIITLKSNLVSQFRKDFQLYKVREINDYHHAHDAYLNAVVATAILKK |
| | YPKLEPEFVYGDYKKYDVRKLISKSDQSMGKATAKMFFYSNLMNFFKKKVRLSDGTVIVRPVIEY |
| | SKDTGEIAWNKEKDFATVRKVLSCPQVNIVKKTEVQTGGFSKESILPKRNSDKLIARKKDWDPKK |
| | YGGFDSPTVAYSVLVVAKVEKGKSKKLKSVKELLGITIMERSSFEKNPIDFLEAKGYKEVRKDLIV |
| | KLPKYSLFELENGRKRMLASAGELQKGNELALPSKYVNFLYLASHYEKLKGSPEDNEQKQLFVEQ |
| | HKHYLDEIIEQISEFSKRVILADANLDKVLSAYNKHRDKPIREQAENIIHLFTLTNLGAPAAFKYFDT |
| | TIDRKRYTSTKEVLDATFIHQSITGLYETRIDLSQLGGD |
| 171 | MTKPYSIGLDIGTNSVGWAVITDSYKVPSKKMKVLGNTSKKYVKKNLLGALLFESGITAEGRRLK |
| | RTARRRYTRRRNRILYLQEIFATEMAKIDESFFQRLDDSFLVPDDKRDSKYPIFGNLVEEKTYHDEF |
| | PTIYHLRKYLADSSKKADLRLVYLALAHMIKYRGHFLIEGDFNSRNNDIKKNFQNFLDSYNAIFESD |
| | LSLENSKQLEEIVKDKISKSAKRERVLKLFPDEKSTGLFSEFLKLIVGNQADFKKHFDLEEKAPLQFS |
| | KDTYDEDLENLLGQIGDGFADLFLVAKKLYDAILLSGILTVTDPSTKAPLSASMIERYENHQKDLA |
| | ALKQFIQNNLQEKYDEVFSDQSKDGYAGYIDGKTTQEAFYKYIKNLLSKLEGADYFLDKIEREDFL |
| | RKQRTFDNGSIPHQIHLQEMNAILRRQGEHYPFLKENKEKIEKILTFRIPYYVGPLARGNRDFAWLT |
| | RNSDQAIRPWNFEEIVDQASSAEDFINKMTNYDLYLPEEKVLPKHSLLYETFAVYNELTKVKFIAEG |
| | LRDYQFLDSGQKKQIVNQLFKEKRKVTEKDIIQYLHNVDGYDGIELKGIEKQFNASLSTYHDLLKII |
| | KDKEFMDDPKNEEILENIVHTLTIFEDREMIKQRLAQYDSLFDEKVIKALSRRHYTGWGRLSAKLIN |
| | GIHDKQSRKTILDYLIDDDKINRNFMQLINDDGLSFKEIIQKAQVVGKTDDLKQVVQNLPGSPAIKK |
| | GILQSIKIVDELVKVMGREPESIVVEMARENQFTNQGRRNSQQRYKKIKDAIKNLDSNLNSKILKEY |
| | PTNNQALQNDRLFLYYLQNGKDMYTGEALDINQLSNYDIDHIIPQAFIKDDSLDNRVLTSSKDNRG |
| | KSDNVPSIEIVEKMKGFWQQLLDSKLISERKFNNLTKAERGGLDERDKVGFIRRQLVETRQITKHV |
| | AQILDARFNTEVNDKKQRIRKVKIVTLKSNLVSNFRKEFGLYKVREINDYHHAHDAYLNAVVAKA |
| | ILKKYPKLEPEFVYGDYQKYDLKRYISSSNPSKEIDKATEKYFFYSNLLNFFKEEVHYADGTIVKRE |
| | NIEYSKDTGEIAWNKEKDFATVRKVLSCPQVNIVKKTEVQTGGFSKESILPKRNSDKLIARKKDWD |
| | PKKYGGFDSPTVAYSVLVVAKVEKGKSKKLKSVKELLGITIMERSSFEKNPIDFLEAKGYKEVRKD |
| | LIVKLPKYSLFELENGRKRMLASAGELQKGNELALPSKYVNFLYLASHYEKLKGSPEDNEQKQLFV |
| | EQHKHYLDEIIEQISEFSKRVILADANLDKVLSAYNKHRDKPIREQAENIIHLFTLTNLGAPAAFKYF |
| | DTTIDRKRYTSTKEVLDATFIHQSITGLYETRIDLSQLGGD |
| 172 | MNKPYSIGLDIGTNSVGWAVITDNYKVPSKKMKVLGNTSKKYVKKNLLGALLFESGITAEGRRLK |
| | RTARRRYTRRRNRILYLQEIFATEMAKIDESFFQRLDDSFLVPDDKRDSKYPIFGNLAQEKTYHDEF |
| | PTIYHLRKYLADSSKKADLRLVYLALAHMIKYRGHFLIEGDFNSRNNDIKKNFQNFLDSYNAIFESD |
| | LSLENSKQLEEIVKDKISKSAKRERVLKLFPDEKSTGLFSEFLKLIVGNQADFKKHFDLEEKAPLQFS |
| | KDTYDEDLENLLGQIGDDFADLFLVAKKLYDAILLSGILTVTDPSTKAPLSASMIERYENHQKDLA |
| | ALKQFIQNNLQEKYDEVFSDQSKDGYAGYIDGKTTQEAFYKYIKNLISKIEGADYFLEKIEREDFLR |
| | KQRTFDNGSIPHQIHLQEMNAILHNQGDYYPFLKENKEKIQQILTFRIPYYVGPLARGNRDFAWLTR |
| | NSDQAIRPWNFEEIVDKASSAEDFINKMTNYDLYLPEEKVLPKHSLLYETFAVYNELTKVKFIAEGL |
| | RDYQFLDSGQKKQIVNQLFKEKRKVTEKDIIQYLHNVDGYDGIELKGIEKQFNASLSTYHDLLKIIK |
| | DKEFMDDPKNEEILENIIHTLTIFEDREMIKQRLAQYDSLFDEKVIKALTRRHYTGWGKLSAKLINGI |
| | RDKQSNKTILDYLIDDDKINRNFMQLINDDGLSFKEIIQKAQVVGKTDDVKQVVQELPGSPAIKKGI |
| | LQSIKLVDELVKVMGSDPESIVIEMARENQTTNQGRRNSQQRYKKIEEAIKNLDSNLNSKILKEYPT |
| | DNQALQNDRLFLYYLQNGKDMYTGEELDINQLSNYDIDHIVPQAFIKDDSLDNRVLTSSKDNRGK |
| | SDNVPSIEVVQKRKGFWQQLLDSKLISERKENNLTKAERGGLDERDKVGFIKRQLVETRQITKHVA |
| | QILDARFNTEVNENNQKIRKVKIITLKSNLVSNFRKEFGLYKVREINDYHHAHDAYLNAVVAKAIL |
| | KKYPKLEPEFVYGDYQKYDVKRYISRSKDPKEIEKATEKYFFYSNLLNFFKEEVHYADGTIIKRENI |
| | EYSNDTGEIAWNKEKDFATVRKVLSCPQVNIVKKTEVQTGGFSKESILPKRNSDKLIARKKDWDPK |
| | KYGGFDSPTVAYSVLVVAKVEKGKSKKLKSVKELLGITIMERSSFEKNPIDFLEAKGYKEVRKDLI |
| | VKLPKYSLFELENGRKRMLASAGELQKGNELALPSKYVNFLYLASHYEKLKGSPEDNEQKQLFVE |
| | QHKHYLDEIIEQISEFSKRVILADANLDKVLSAYNKHRDKPIREQAENIIHLFTLTNLGAPAAFKYFD |
| | TTIDRKRYTSTKEVLDATFIHQSITGLYETRIDLSQLGGD |
| 173 | MTKPYSIGLDIGTNSVGWAVITDSYKVPSKKMKVLGNTSKKYVKKNLLGALLFESGITAEGRRLK |
| | RTARRRYTRRRNRILYLQEIFSTEMAKIDESFFQRLDDSFLVPDDKRDSKYPIFGNLVEEKVYHDEF |
| | PTIYHLRKYLADSSKKADLRLVYLALAHMIKYRGHFLIEGDFNSRNNDIKKNFQEFLDSYNAIFESD |
| | LSLENNAQVEEIVKDKISKSAKRERVLKLFPDEKSTGLFSEFLKLIVGNQADFKKHFDLEEKAPLQF |
| | SKDTYDEDLENLLGQIGDDFADLFLVAKKLYDAILLSGILTVTDPSTKAPLSASMIERYENHQKDLA |
| | QLKQFIKASLPEKYYDVFADESKNGYAGYIENGVKQDEFYKYLKNTLNKIEGSDYFLDKIEREDFL |
| | RKQRTFDNGSIPHQIHLQEMNAIIRRQGEHYPFLQENKEKIEKILTFRIPYYVGPLARGNRDFAWLTR |
| | NSDQAIRPWNFEEVVDKASSAEDFINKMTNYDLYLPEEKVLPKHSLLYETFAVYNELTKVKFIAEG |
| | LRDYQFLDSGQKKQIVNQLFKEKRKVTEKDIIHYLHNVDGYDGIELKGIEKQFNASLSTYHDLLKII |
| | KDKEFMDDPKNEEILENIVHTLTIFEDREMIKQRLAQYDSLFDEKVIKALTRRHYTGWGKLSAKLIN |
| | GICDKQTGNTILDYLIDDDKINRNFMQLINDDGLSFKEIIQKAQVVGKTDDVKQVVQELPGSPAIKK |
| | GILQSIKIVDELVKVMGHAPESIVIEMARENQTTARGKKNSQQRYKRIEDALKNLAPGLDSNILKEN |
| | PTDNIQLQNDRLFLYYLQNGKDMYTGKELDINQLSNYDIDHIVPQAFIKDDSLDNRVLTSSKDNRG |
| | KSDNVPSIEVVQKRKAFWQQLLDSKLISERKENNLTKAERGGLNELDKVGFIKRQLVETRQITKHV |
| | AQILDARFNTEVDENNQRIRKVKIITLKSNLVSNFRKEFGLYKVREINDYHHAHDAYLNAVVAKAI |
| | LKKYPKLEPEFVYGDYQKYDLKRYISRSKDPKDIEKATEKYFFYSNLLNFFKEEVHYADGTIVKRE |
| | NIEYSKDTGEIAWNKEKDFATVRKVLSCPQVNIVKKTEVQTGGFSKESILPKRNSDKLIARKKDWD |
| | PKKYGGFDSPTVAYSVLVVAKVEKGKSKKLKSVKELLGITIMERSSFEKNPIDFLEAKGYKEVRKD |
| | LIVKLPKYSLFELENGRKRMLASAGELQKGNELALPSKYVNFLYLASHYEKLKGSPEDNEQKQLFV |
| | EQHKHYLDEIIEQISEFSKRVILADANLDKVLSAYNKHRDKPIREQAENIIHLFTLTNLGAPAAFKYF |
| | DTTIDRKRYTSTKEVLDATFIHQSITGLYETRIDLSQLGGD |
| 174 | MTKPYSIGLDIGTNSVGWAVITDSYKVPSKKMKVLGNTSKKYVKKNLLGALLFESGITAEGRRLK |
| | RTARRRYTRRRNRILYLQEIFSTEMAKVDESFFQRLDDSFLVPDDKRDSKYPIFGNLVEEKTYHDEF |
| | PTIYHLRKYLADSSKKADLRLVYLALAHMIKYRGHFLIEGDFNSRNNDIKKNFQNFLDSYNAIFESD |
| | LSLENSEQLEEIVKDKISKSAKRERVLKLFPDEKSTGLFSEFLKLIVGNQAEFKKHFDLEEKAPLQFS |
| | KDTYDEDLENLLGQIGDGFADLFLVAKKLYDAILLSGILTVTDPSTKAPLSASMIERYENHQKDLA |
| | ALKQFIQNNLQEKYDEVFSDQSKDGYAGYIDGKTTQEAFYKYIKNLLSKFEGADYFLDKIEREDFL |
| | RKQRTFDNGSIPHQIHLQEMNAILRRQGEHYPFLKENKEKIQQILTFRIPYYVGPLARGNRDFAWLT |
| | RNSDQAIRPWNFEEVVDKASSAEDFINKMTNYDLYLPEEKVLPKHSLLYETFAVYNELTKVKFIAE |
| | GLRDYQFLDSGQKKQIVNQLFKEKRKVTEKDIIHFLHNVDGYDGIELKGIEKQFNASLSTYHDLLKI |
| | IKDKDFMDDPKNEEILENIVHTLTIFEDREMIKQRLAQYDSLFDEKVIKALTRRHYTGWGKLSAKLI |
| | NGICDKQTGDTILDYLIDDDKINRNFMQLIHDDGLSFKEIIQKAQVFGKTNDVKQVVQELPGSPAIK |
| | KGILQSIKIVDELVKVMGHAPESIVIEMARENQTTNQGRRNSQQRYKLIEDGVKNLASDLNGNILKE |
| | YPTDNQALQNDRLFLYYLQNGKDMYTGNPLDINQLSNYDIDHIVPQAFIKDDSLDNRVLTSSKDNR |
| | GKSDNVPSLEVVEKMKAFWQQLLDSKLISERKYNNLTKAERERGGLNELDKVGFIKRQLVETRQI |
| | TKHVAQILDARFNTEVDENNQKIRKVKIITLKSNLVSNFRKEFGLYKVREINDYHHAHDAYLNAVV |
| | AKAILKKYPKLEPEFVYGDYQKYDLKRYISRSKDPKEIEKATEKYFFYSNLLNFFKEEVHYADGTII |
| | KRENIEYSKDTGEIAWNKEKDFATVRKVLSCPQVNIVKKTEVQTGGFSKESILPKRNSDKLIARKK |
| | DWDPKKYGGFDSPTVAYSVLVVAKVEKGKSKKLKSVKELLGITIMERSSFEKNPIDFLEAKGYKEV |
| | RKDLIVKLPKYSLFELENGRKRMLASAGELQKGNELALPSKYVNFLYLASHYEKLKGSPEDNEQK |
| | QLFVEQHKHYLDEIIEQISEFSKRVILADANLDKVLSAYNKHRDKPIREQAENIIHLFTLTNLGAPAA |
| | FKYFDTTIDRKRYTSTKEVLDATFIHQSITGLYETRIDLSQLGGD |
| 175 | MNKPYSIGLDIGTNSVGWSVVDDDYKVPAKKMRVFGDTDKGHIKKNLLGTLLFDSGETAESRRLK |
| | RTARRRYTRRRNRILYLQEIFAKEMAKVDESFFHRLDESFLTDDDKTFDSHPIFGNKAEEDAYHQQ |
| | FPTIYHLRKHLADSTEKADLRLVYLALAHMIKFRGHFLIEGELNAENTDVQKLFADFVGVYDRTFD |
| | DSHLSEITVDASSILTEKISKSRRLEKLIKHYPTEKKNTLFGNLVALALGLQPNFKTNFKLSEDAKLQ |
| | FSKDTYEEDLEELLGKIGDDYADLFTSAKNLYDAILLSGILTVDDESTKAPLSASMIKRYAEHHEDL |
| | EKLKEFVKANKPELYHDIFKDESKNGYAGYIENGVKQDEFYKYLKETLSKQDGSQYFLDKIEREDF |
| | LRKQRTFDNGSIPHQLHLKEMRSILHRQGEYYPFLKENQAKIEKILTFRIPYYVGPLARKDSRFAWA |
| | EYHSDEPITPWNFDEVVDKEKSAEKFITRMTLNDLYLPEEKVLPKHSHVYETYTVYNELTKVKYV |
| | NEQGEAKFFDANMKQEIFDHVFKENRKVTKDKLMDFLGKEFDEFRIVDITGLDKENKAFNASLGT |
| | YHDLKKILDKSFLDDKENETIIEDIIQTLTLFEDRDMIRQRLQKYSDIFTPQQLKKLERRHYTGWGRL |
| | SYKLINGIRDKQTGKTILDYLIDDGYANRNFMQLINDDTLPFKEIIQKAQIIGDIDDIKAVVHDLPGSP |
| | AIKKGILQSVKIVDELVKVMGHNPDNIVIEMARENQTTNRGRTQSQQRLKKLQESLKELGSNILNE |
| | KKPSYVEDKVENTHLQNDRLFLYYIQNGKDMYTGDELDIDHLSDYDIDHIIPQAFIKDDSIDNRVLT |
| | SSAKNRGKSDDVPSIEIVRDRKADWMRLRKAGLISKRKFDNLTKAERGGLTEADKAGFIKRQLVET |
| | RQITKHVAQILDSRENTERDENDKVIREVKVITLKSNLVSQFRKDFQFYKVREINDYHHAHDAYLN |
| | AVVGTALIKKYPKLESEFVYGDYKVYDVRKMIAKSEQEIGKATAKRFFYSNIMNFFKTEIKLANGE |
| | IRKRPLIETNEETGEIVWNKEKDFATVRKVLSCPQVNIVKKTEVQTGGFSKESILPKRNSDKLIARKK |
| | DWDPKKYGGFDSPTVAYSVLVVAKVEKGKSKKLKSVKELLGITIMERSSFEKNPIDFLEAKGYKEV |
| | RKDLIVKLPKYSLFELENGRKRMLASAGELQKGNELALPSKYVNFLYLASHYEKLKGSPEDNEQK |
| | QLFVEQHKHYLDEIIEQISEFSKRVILADANLDKVLSAYNKHRDKPIREQAENIIHLFTLTNLGAPAA |
| | FKYFDTTIDRKRYTSTKEVLDATFIHQSITGLYETRIDLSQLGGD |
| 176 | MTKPYSIGLDIGTNSVGWAVITDSYKVPSKKMKVLGNTSKKYVKKNLLGALLFESGITAEGRRLK |
| | RTARRRYTRRRNRILYLQEIFSTEMAKIDENFFQRLDDSFLVPDDKRDSKYPIFGNLVEEKVYHDEF |
| | PTIYHLRKYLADSSKKADLRLVYLALAHMIKYRGHFLIEGDFNSRNNDIKKNFQDFLDSYNAIFESD |
| | LSLENSKQLEEIVKDKISKSAKRERVLSLFPDEKSTGLFSEFLKLIVGNQADFKKHFDLEEKAPLQFS |
| | KDTYDEDLENLLGQIGDDFADLFLVAKKLYDAILLSGILTVTDPSTKAPLSASMIDRYENHQKDLA |
| | ALKQFVKANLPEKYDEVFSDQSKDGYAGYIDGKTTQEAFYKYIKNTLSKIEGADYFLDKIEREDFL |
| | RKQRTFDNGSIPHQIHLQEMNAIIRRQGEHYPFLQENKEKIEKILTFRIPYYVGPLARGNRDFAWLTR |
| | NSDQAIRPWNFEEIVDQASSAEDFINKMTNYDLYLPEEKVLPKHSLLYETFAVYNELTKVKFIAEGL |
| | RDYQFLDSGQKKQIVNQLFKEKRKVTEKDIIQFLHNVDGYDGIELKGIEKQFNASLSTYHDLLKIIK |
| | DKEFMDDPKNEEILENIVHTLTIFEDREMIKQRLAQYDTLFDEKVIKALTRRHYTGWGKLSAKLIN |
| | GICDKQTGDTILDYLIDDDKINRNFMQLINDDGLSFKDIIQKAQVVGKTDDVKQVVQELPGSPAIKK |
| | GILQSIKIVDELVKVMGHAPESIVIEMARENQTTARGKKNSQQRYKRIEDALKNLAPGLDSNILKEN |
| | PTDNIQLQNDRLFLYYLQNGKDMYTGEALDINQLSNYDIDHIVPQAFIKDDSLDNRVLTSSKDNRG |
| | KSDNVPSLEVVQKRKAFWQQLLDSKLISERKENNLTKAERGGLDERDKVGFIKRQLVETRQITKHV |
| | AQILDARFNTEVNEKNQKIRKVKIVTLKSNLVSNFRKEFKLYKVREINDYHHAHDAYLNAVVAKA |
| | ILKKYPKLEPEFVYGDYQKYDVKRYISRKELTTSQDKATAKYFFYSNLLNFFKKEVRLADGTIVVR |
| | PVIEYSKDTGEIAWNKEKDFATVRKVLSCPQVNIVKKTEVQTGGFSKESILPKRNSDKLIARKKDW |
| | DPKKYGGFDSPTVAYSVLVVAKVEKGKSKKLKSVKELLGITIMERSSFEKNPIDFLEAKGYKEVRK |
| | DLIVKLPKYSLFELENGRKRMLASAGELQKGNELALPSKYVNFLYLASHYEKLKGSPEDNEQKQLF |
| | VEQHKHYLDEIIEQISEFSKRVILADANLDKVLSAYNKHRDKPIREQAENIIHLFTLTNLGAPAAFKY |
| | FDTTIDRKRYTSTKEVLDATFIHQSITGLYETRIDLSQLGGD |
| 177 | MNKKYSIGLDIGTNSVGWAVVDDDYKVPAKKMRVFGDTDRSHIKKNLLGALLFDSGETAESRRL |
| | KRTARRRYTRRRNRILYLQEIFAEEMAKVDESFFHRLDESFLTDDDKTFDSHPIFGNKAEEDAYHK |
| | QFPTIYHLRKHLADSSEKADLRLVYLALAHMIKFRGHFLIEGELNAENTDVQKLFEQFVGVYDRTF |
| | DDSHLSEITVDASSILTEKISKSRRLEKLIKHYPTEKKNTLFGNLIALSLGLQPNFKTNFKLSEDAKLQ |
| | FSKDTYEEDLEELLGKIGDDYADLFTLAKNLYDAILLSGILTVDDESTKAPLSASMIKRYAEHHEDL |
| | EKLKEFVKANKPELYHDIFKDETKNGYAGYIENGVKQDEFYKYLKNTLSKIDGSQYFLDKIEREDF |
| | LRKQRTFDNGSIPHQIHLQEMHAILHRQGEYYPFLKENQAKIEKILTFRIPYYVGPLARKNSRFAWA |
| | NYHSDEPITPWNFDEVVDKEKSAEKFITRMTLNDLYLPEEKVLPKHSLVYETYTVYNELTKVKYV |
| | NEQGKESFFDANMKQEIFDHVFKENRKVTKDKLLNYLDKEFPEYRIQDLIGLDKENKSFNASLGTY |
| | HDLKKILDKSFLDDKENEQIIEDIIQTLTLFEDRDMIRQRLQKYSDIFTKQQLKKLERRHYTGWGRL |
| | SYKLINGIRDKKSGKSILDYLIDDGYANRNFMQLINDDTLPFKQIIQKAQVVGDIDDIKAVVHDLPG |
| | SPAIKKGILQSVKIVDELVKVMGHNPDNIVIEMARENQTTNRGRTQSQQRLKKLQESLKDLGSNIL |
| | NEEKPSYIEDKVENSHLQNDKLFLYYIQNGKDMYTGDELDIDHLSDYDIDHIVPQAFIKDDSIDNRV |
| | LTSSSKNRGKSDDVPSIEVVCARKADWMRLRKAGLISQRKFDNLTKAERGGLTEHDKAGFIKRQL |
| | VETRQITKHVAQILDSRFNTKRDDNDKVIREVKVITLKSNLVSQFRKDFQFYKVREINDYHHAHDA |
| | YLNAVVGTALIKKYPKLESEFVYGDYKVYDVRKMIAKSEQEIGKATAKRFFYSNIMNFFKTEIKLA |
| | NGEIRKRPLIEVNEETGEIVWNKEKDFATVRKVLSCPQVNIVKKTEVQTGGFSKESILPKRNSDKLI |
| | ARKKDWDPKKYGGFDSPTVAYSVLVVAKVEKGKSKKLKSVKELLGITIMERSSFEKNPIDFLEAK |
| | GYKEVRKDLIVKLPKYSLFELENGRKRMLASAGELQKGNELALPSKYVNFLYLASHYEKLKGSPE |
| | DNEQKQLFVEQHKHYLDEIIEQISEFSKRVILADANLDKVLSAYNKHRDKPIREQAENIIHLFTLTNL |
| | GAPAAFKYFDTTIDRKRYTSTKEVLDATFIHQSITGLYETRIDLSQLGGD |
| 178 | MTKPYSIGLDIGTNSVGWAVITDSYKVPSKKMKVLGNTSKKYVKKNLLGALLFESGITAEGRRLK |
| | RTARRRYTRRRNRILYLQEIFSTEMSKIDESFFQRLDDSFLVPDDKRDSKYPIFGNLVEEKAYHDEFP |
| | TIYHLRKYLADSSKKADLRLVYLALAHMIKYRGHFLIEGDFNSKNNSIKKTFQQFLDSYNAIFEDDL |
| | SLKNSEQLEEIVKDKISKSAKRERVLKLFPDEKSTGLFSEFLKLIVGNQADFKKHFDLEEKAPLQFSK |
| | DTYDEDLENLLGQIGDDFADLFLVAKKLYDAILLSGILTVTDPSTKAPLSASMIERYENHQKDLAAL |
| | KQFIQNNLQEKYDEVFSDQSKDGYAGYIDGKTTQEAFYKYIKETLSKIEGSDYFLDKIEREDFLRKQ |
| | RTFDNGSIPHQIHLQEMNAILRRQGEHYPFLKENKEKIEKILTFRIPYYVGPLARGNRDFAWLTRNS |
| | DQAIRPWNFEEVVDQASSAEEFINKMTNYDLYLPEEKVLPKHSLLYETFAVYNELTKVKFIAEGLR |
| | DYQFLDSGQKKQIVNQLFKEKRKVTEKDIIQFLHNVDGYDGIELKGIEKQFNASLSTYHDLLKIIKD |
| | KEFMDDPKNEEILENIVHTLTIFEDREMIKQRLAQYDSLFDEKVIKALTRRHYTGWGKLSAKLINGI |
| | YDKQSKKTILDYLIDDDKINRNFMQLINDDGLSFKDIIQKAQVVGKTDDVKQVVQELPGSPAIKKGI |
| | LQSIKLVDELVKVMGHAPESIVIEMARENQTTARGKKNSQQRYKRIEDALKNLAPGLDSNILKENP |
| | TDNVQLQNDRLFLYYLQNGKDMYTGEALDINQLSNYDIDHIVPQAFIKDDSLDNRVLTSSKDNRG |
| | KSDNVPSLEVVQKRKGFWQQLLDSKLISERKENNLTKAERGGLDERDKVGFIRRQLVETRQITKHV |
| | AQILDERFNTEVDSKGKKIRKVKIITLKSNLVSNFRKEFGLYKVREINDYHHAHDAYLNAVVAKAI |
| | LKKYPKLEPEFVYGDYQKYDIKRYISRSKDPKEVEKATEKYFFYSNLLNFFKEEVHYADGTIVKRE |
| | NIEYSNDTGEIAWNKEKDFATVRKVLSCPQVNIVKKTEVQTGGFSKESILPKRNSDKLIARKKDWD |
| | PKKYGGFDSPTVAYSVLVVAKVEKGKSKKLKSVKELLGITIMERSSFEKNPIDFLEAKGYKEVRKD |
| | LIVKLPKYSLFELENGRKRMLASAGELQKGNELALPSKYVNFLYLASHYEKLKGSPEDNEQKQLFV |
| | EQHKHYLDEIIEQISEFSKRVILADANLDKVLSAYNKHRDKPIREQAENIIHLFTLTNLGAPAAFKYF |
| | DTTIDRKRYTSTKEVLDATFIHQSITGLYETRIDLSQLGGD |
| 179 | MTKPYSIGLDIGTNSVGWAVITDSYKVPSKKMKVLGNTSKKYVKKNLLGALLFESGITAEGRRLK |
| | RTARRRYTRRRNRILYLQEIFIAEMNKIDESFFQRLDDSFLVPDDKRGSKYPIFGNLAKEKTYHDEFP |
| | TIYHLRKYLADSTKKADLRLVYLALAHMIKYRGHFLIEGDFNSRNNDIKKNFQNFLDSYNAIFESD |
| | LSLENSKQLEEIVKDKISKSAKRERVLKLFPDEKSTGLFSEFLKLIVGNQADFKKHFDLEEKAPLQFS |
| | KDTYDEDLENLLGQIGDDFADLFLVAKKLYDAILLSGILTVTDPSTKAPLSASMIERYENHQKDLA |
| | ALKQFIQNNLQEKYDEVFSDQSKDGYAGYIDGKTTQEAFYKYIKNLLSKFEGADYFLEKIKREDFL |
| | RKQRTFDNGTIPYQVHLTEMKAILHRQGDYYPFLKENKEKIEQILTFRIPYYVGPLARGNRDFAWL |
| | TRNSDQAIRPWNFEEVVDQASSAEDFINKMTNYDLYLPEEKVLPKHSLLYETFAVYNELTKVKFIA |
| | EGLRDYQFLDSGQKKQIVNQLFKEKRKVTEKDIIHFLHNVDGYDGIELKGIEKQFNASLSTYHDLL |
| | KIIKDKEFMDDAKNEAILENIIHTLTIFEDREMIKQRLAQYDSLFDEKVIKALTRRHYTGWGKLSAK |
| | LINGIRDKQTGKTILDYLIDDGEINRNFMQLIHDDGLSFKEIIQKAQVVGKTDDLKQVVQELPGSPAI |
| | KKGILQSIKIVDELVKVMGHAPESIVIEMARENQTTNQGKRNSQQRYKRIEDGVKNLASDLNGNIL |
| | KEYPTDNQALQNDRLFLYYLQNGKDMYTGEPLDINQLSNYDIDHIVPQAFIKDDSLDNRVLTSSKD |
| | NRGKSDNVPSIEVVEKMKGFWQQLLDSKLISERKFNNLTKAERGGLDERDKVGFIKRQLVETRQIT |
| | KHVAQILDARFNTEVDEKGKKIRKVKIITLKSNLVSNFRKEFGLYKVREINDYHHAHDAYLNAVV |
| | AKAILKKYPKLEPEFVYGDYQKYDLKRYISKSKNPKEIDKATEKYFFYSNLLNFFKEEVHYADGTII |
| | KRENIEYSKDTGEIAWNKEKDFATVRKVLSCPQVNIVKKTEVQTGGFSKESILPKRNSDKLIARKK |
| | DWDPKKYGGFDSPTVAYSVLVVAKVEKGKSKKLKSVKELLGITIMERSSFEKNPIDFLEAKGYKEV |
| | RKDLIVKLPKYSLFELENGRKRMLASAGELQKGNELALPSKYVNFLYLASHYEKLKGSPEDNEQK |
| | QLFVEQHKHYLDEIIEQISEFSKRVILADANLDKVLSAYNKHRDKPIREQAENIIHLFTLTNLGAPAA |
| | FKYFDTTIDRKRYTSTKEVLDATFIHQSITGLYETRIDLSQLGGD |
| 180 | MNKPYSIGLDIGTNSVGWAVITDSYKVPSKKMKVLGNTSKKYVKKNLLGALLFESGITAEGRRLK |
| | RTARRRYTRRRNRILYLQEIFAAEMNKIDESFFQRLDDSFLVPDDKRGSKYPIFGNLAKEKTYHDEF |
| | PTIYHLRKYLADSSKKADLRLVYLALAHMIKYRGHFLIEGDFNSKNNDIKKNFQEFLDSYNAIFESD |
| | LSLENSKQLEEIVKDKISKSAKRERVLKLFPDEKSTGLFSEFLKLIVGNQADFKKHFDLEEKAPLQFS |
| | KDTYDEDLENLLGQIGDDFADLFLVAKKLYDAILLSGILTVTDPSTKAPLSASMIERYENHQKDLAT |
| | LKQFIKTNLPEKYDEVFSDQSKDGYAGYIDGKTTQEAFYKYIKNLLSKIEGADYFLEKIEREDFLRK |
| | QRTFDNGSIPHQIHLQEMNAILRRQGEHYPFLKENKEKIQQILTFRIPYYVGPLARGNRDFAWLTRN |
| | SDQAIRPWNFEEIVDQASSAEDFINKMTNYDLYLPEEKVLPKHSLLYETFAVYNELTKVKFIAEGLR |
| | DYQFLDSGQKKQIVNQLFKEKRKVTEKDIIHYLHNVDGYDGIELKGIEKQFNASLSTYHDLLKIIKD |
| | KEFMDDPKNEEILENIVHTLTIFEDREMIKQRLAQYDSLFDEKVIKALTRRHYTGWGKLSAKLINGI |
| | YDKQSKKTILDYLIDDGEINRNFMQLINDDGLSFKEIIQKAQVVGKTDDVKQVVQELPGSPAIKKGI |
| | LQSIKLVDELVKVMGHAPESIVIEMARENQTTARGKKNSQQRYKRIEDALKNLAPGLDSNILKENP |
| | TDNIQLQNDRLFLYYLQNGKDMYTGEALDINQLSSYDIDHIVPQAFIKDDSLDNRVLTSSKDNRGK |
| | SDNVPSLEVVQKRKAFWQQLLDSKLISERKENNLTKAERGGLTEHDKAGFIKRQLVETRQITKHVA |
| | QILDERFNTEVNEEGKRIRKVKIITLKSNLVSNFRKEFGLYKVREINDYHHAHDAYLNAVVAKAILK |
| | KYPKLEPEFVYGEYKKYHIKSSLNSKTIDKATEKYFFYSNLLNFFKEEVHYADGTIIKRENIEYSKDT |
| | GEIAWNKEKDFATVRKVLSCPQVNIVKKTEVQTGGFSKESILPKRNSDKLIARKKDWDPKKYGGF |
| | DSPTVAYSVLVVAKVEKGKSKKLKSVKELLGITIMERSSFEKNPIDFLEAKGYKEVRKDLIVKLPKY |
| | SLFELENGRKRMLASAGELQKGNELALPSKYVNFLYLASHYEKLKGSPEDNEQKQLFVEQHKHYL |
| | DEIIEQISEFSKRVILADANLDKVLSAYNKHRDKPIREQAENIIHLFTLTNLGAPAAFKYFDTTIDRKR |
| | YTSTKEVLDATFIHQSITGLYETRIDLSQLGGD |
| 181 | MTKPYSIGLDIGTNSVGWAVISDSYKVPSKKMKVLGNTSKKYVKKNLLGALLFESGITAEGRRLKR |
| | TARRRYTRRRNRILYLQEIFSTEMAKIDESFFQRLDDSFLVPDDKRDSKYPIFGNLVEEKTYHDEFPT |
| | IYHLRKYLADSSKKADLRLVYLALAHMIKYRGHFLIEGDFNSRNNDIKKNFQNFLDSYNAIFESDLS |
| | LENSKQLEEIVKDKISKSAKRERVLKLFPDEKSTGLFSEFLKLIVGNQADFKKHFDLEEKAPLQFSK |
| | DTYDEDLENLLGQIGDDFADLFLVAKKLYDAILLSGILTVTDPSTKAPLSASMIERYENHQKDLAAL |
| | KQFIQNNLQEKYDEVFSDQSKDGYAGYINGKTTQEAFYKYIKNLLSKLEGADYFLDKIEREDFLRK |
| | QRTFDNGSIPHQIHLQEMNAIIRRQGEHYPFLKENKEKIEKILTFRIPYYVGPLARGNRDFAWLTRNS |
| | DQAIRPWNFEEVVDKASSAEDFINKMTNYDLYLPEEKVLPKHSLLYETFAVYNELTKVKFIAEGLR |
| | DYQFLDSGQKKQIVNQLFKEKRKVTEKDIIHYLHNVDGYDGIELKGIEKQFNASLSTYHDLLKIIKD |
| | KAFMDDAKNEEILENIVHTLTIFEDREMIKQRLAQYDSLFDEKVIKALTRRHYTGWGKLSARLIDGI |
| | YDKQSRKTILDYLIDDGEINRNFMQLIHDDGLSFKDIIQKAQVVGKTDDVKQVVQELPGSPAIKKGI |
| | LQSIKIVDELVKVMGHAPESIVIEMARENQTTARGKKNSQQRYKRIEDALKNLAPGLDSNILKENPT |
| | DNIQLQNDRLFLYYLQNGKDMYTGEALDINQLSNYDIDHIVPQAFIKDDSLDNRVLTSSKDNRGKS |
| | DNVPSLEVVQRRKAFWQQLLDSKLISERKENNLTKAERGGLTEHDKAGFIKRQLVETRQITKHVA |
| | QILDARFNTEVDEKGKRIRKVKIITLKSNLVSNFRKEFGLYKVREINDYHHAHDAYLNAVVAKAIL |
| | KKYPKLEPEFVYGDYQKYDIKRYISRSKDSKDIEKATEKYFFYSNLLNFFKEEVHYADGTIVKRENI |
| | EYSNDTGEIAWNKEKDFATVRKVLSCPQVNIVKKTEVQTGGFSKESILPKRNSDKLIARKKDWDPK |
| | KYGGFDSPTVAYSVLVVAKVEKGKSKKLKSVKELLGITIMERSSFEKNPIDFLEAKGYKEVRKDLI |
| | VKLPKYSLFELENGRKRMLASAGELQKGNELALPSKYVNFLYLASHYEKLKGSPEDNEQKQLFVE |
| | QHKHYLDEIIEQISEFSKRVILADANLDKVLSAYNKHRDKPIREQAENIIHLFTLTNLGAPAAFKYFD |
| | TTIDRKRYTSTKEVLDATFIHQSITGLYETRIDLSQLGGD |
| 182 | MKKPYSIGLDIGTNSVGWAVITDDYKVPSKKMKVLGNTSKKYVKKNLLGALLFESGITAEGRRLK |
| | RTARRRYTRRRNRILYLQEIFSTEMAQIDDSFFQRLDDSFLVPDDKRDSKYPIFGNLVEEKVYHDEF |
| | PTIYHLRKYLADSQKKADLRLVYLALAHMIKYRGHFLIEGDFNSRNNDIKKNFQDFLDSYNAIFES |
| | DLSLENSKQLEEIVKDKISKSAKRERVLKLFPDEKSTGLFSEFLKLIVGNQADFKKHFDLEEKAPLQF |
| | SKDTYDEDLENLLGQIGDDFADLFLVAKKLYDAILLSGILTVTDPSTKAPLSASMIERYENHQKDLA |
| | ALKQFIQNNLQEKYDEVFSDQSKDGYAGYIDGKTTQEAFYKYIKNLLSKFEGADYFLEKINRDDLL |
| | RKQRTFDNGSIPHQIHLQEMHAIIRRQGEYYPFLKENKEKIEKILTFRIPYYVGPLARGNRDFAWLTR |
| | NSDQAIRPWNFEEIVDKASSAEDFINKMTNYDLYLPEEKVLPKHSLLYETFAVYNELTKVKFIAEGL |
| | RDYQFLDSGQKKQIVNQLFKEKRKVTEKDIIHYLHNVDGYDGIELKGIEKQFNASLSTYHDLLKIIK |
| | DKEFMDDPKNEEILENIVHTLTIFEDREMIKQRLAQYDSLFDEKVIKALTRRHYTGWGKLSAKLING |
| | ICDKQTGNTILDYLIDDDKINRNFMQLINDDGLSFKEIIQKAQVVGETDNIKQVVQELPGSPAIKKGI |
| | LQSLKIVDELIEVMGYDPEHIVVEMARENQFTNQGRRNSQQRYKKIEDAIKNLDSNLNSKILKEYPT |
| | DNNALQNDRLFLYYLQNGKDMYTGEALDINQLSNYDIDHIIPQAFIKDDSLDNRVLTSSKDNRGKS |
| | DNVPSIEIVEKMKGFWQQLLNSKLISERKENNLTKAERGGLDERDKVGFIKRQLVETRQITKHVAQI |
| | LDARFNTEVNENNQKIRKVKIITLKSNLVSNFRKEFGLYKVREINDYHHAHDAYLNAVVAKAILKK |
| | YPKLEPEFVYGDYQKYDIKRYISRSKDPKDIEKATEKYFFYSNLLNFFKEEVHYADGTIIKRENIEYS |
| | KDTGEIAWNKEKDFATVRKVLSCPQVNIVKKTEVQTGGFSKESILPKRNSDKLIARKKDWDPKKY |
| | GGFDSPTVAYSVLVVAKVEKGKSKKLKSVKELLGITIMERSSFEKNPIDFLEAKGYKEVRKDLIVKL |
| | PKYSLFELENGRKRMLASAGELQKGNELALPSKYVNFLYLASHYEKLKGSPEDNEQKQLFVEQHK |
| | HYLDEIIEQISEFSKRVILADANLDKVLSAYNKHRDKPIREQAENIIHLFTLTNLGAPAAFKYFDTTID |
| | RKRYTSTKEVLDATFIHQSITGLYETRIDLSQLGGD |
| 183 | MTKPYSIGLDIGTNSVGWAVITDSYKVPSKKMKVLGNTSKKYVKKNLLGALLFESGITAEGRRLK |
| | RTARRRYTRRRNRILYLQEIFSTEMAKIDESFFQRLDDSFLVPDDKRDSKYPIFGNLVEEKTYHDEFP |
| | TIYHLRKYLADSQKKADLRLVYLALAHMIKYRGHFLIEGDFNSRNNDIKKNFQNFLDSYNAIFESD |
| | LSLENNAQVEEIVKDKISKSAKRERVLKLFPDEKSTGLFSEFLKLIVGNQADFKKHFDLEEKAPLQF |
| | SKDTYDEDLENLLGQIGDDFADLFLVAKKLYDAILLSGILTVTDPSTKAPLSASMIDRYENHQKDL |
| | AALKQFIKANLPEKYDEVFSDQSKDGYAGYIDGKTTQEAFYKYIKNLLSKFEGADYFLEKIEREDF |
| | LRKQRTFDNGSIPHQIHLQEMNAILRRQGEHYPFLKENKEKIEKILTFRIPYYVGPLARGNRDFAWL |
| | TRNSDQAIRPWNFEEVVDQASSAEDFINKMTNYDLYLPEEKVLPKHSLLYETFAVYNELTKVKFIA |
| | EGLRDYQFLDSGQKKQIVNQLFKEKRKVTEKDIIQFLHNVDGYDGIELKGIEKQFNASLSTYHDLL |
| | KIIKDKEFMDDPKNEEILENIVHTLTIFEDREMIKQRLAQYDSLFDEKVIKALTRRHYTGWGKLSAK |
| | LINGICDKQTGDTILDFLIDDDKINRNFMQLINDDGLSFKEIIQKAQVVGKTDDVKQVVQELPGSPAI |
| | KKGILQSIKIVDELVKVMGYEPEQIVIEMARENQTTNQGRRNSQQRYKLIEDGVKNLASDLDGNIL |
| | KEYPTDNQALQNDRLFLYYLQNGKDMYTGESLDINQLSSYDIDHIVPQAFIKDDSLDNRVLTSSKD |
| | NRGKSDNVPSLEVVQKRKAFWQQLLDSKLISERKENNLTKAERGGLDERDKVGFIKRQLVETRQIT |
| | KHVAQILDARFNTEVNENNQKIRKVKIITLKSNLVSNFRKEFGLYKVREINDYHHAHDAYLNAVV |
| | AKAILKKYPKLEPEFVYGDYQKYDIKRYISRSKDPKAIEKATQKYFFYSNLLNFFKEEVHYADGTII |
| | KRENIEYSKDTGEIAWNKEKDFATVRKVLSCPQVNIVKKTEVQTGGFSKESILPKRNSDKLIARKK |
| | DWDPKKYGGFDSPTVAYSVLVVAKVEKGKSKKLKSVKELLGITIMERSSFEKNPIDFLEAKGYKEV |
| | RKDLIVKLPKYSLFELENGRKRMLASAGELQKGNELALPSKYVNFLYLASHYEKLKGSPEDNEQK |
| | QLFVEQHKHYLDEIIEQISEFSKRVILADANLDKVLSAYNKHRDKPIREQAENIIHLFTLTNLGAPAA |
| | FKYFDTTIDRKRYTSTKEVLDATFIHQSITGLYETRIDLSQLGGD |
| 184 | MTKPYSIGLDIGTNSVGWAVITDSYKVPSKKMKVLGNTSKKYVKKNLLGALLFESGITAEGRRLK |
| | RTARRRYTRRRNRILYLQEIFSTEMAEIDESFFQRLDDSFLVPDDKRDSKYPIFGNLVKEKTYHDEFP |
| | TIYHLRKYLADSSKKADLRLVYLALAHMIKYRGHFLIEGDFNSKNNDIKKNFQEFLDSYNAIFESDL |
| | SLENSKQLEEIVKDKISKSAKRERVLKLFPDEKSTGLFSEFLKLIVGNQADFKKHFDLEEKAPLQFSK |
| | DTYDEDLENLLGQIGDDFADLFLVAKKLYDAILLSGILTVTDPSTKAPLSASMIERYENHQKDLAAL |
| | KQFIKNNLPEKYDEVFSDDSKDGYAGYIDGKTSQEDFYKYLKKLLTGIEGADYFLEKIEREDFLRK |
| | QRTFDNGVIPYQIHLQEMKAILHNQGEYYPFLKENKEKIEQILTFRIPYYVGPLARGNRDFAWLTRN |
| | SDQAIRPWNFEEIVDKASSAEDFINKMTNYDLYLPEEKVLPKHSLLYERFAVYNELTKVKFIAEGLR |
| | DYQFLDSGQKKQIVNQLFKEKRKVTEKDIIHYLHNVDGYDGIELKGIEKQFNASLSTYHDLLKIIKD |
| | KEFMDDPKNEEILENIVHTLTIFEDREMIKQRLAQYDSLFDEKVIKALTRRHYTGWGKLSAKLINGI |
| | CDKQTGDTILDYLIDDDKINRNFMQLIHDDGLSFKEIIQKAQVVGKTDDLKQVVQELPGSPAIKKGI |
| | LQSIKIVDELVKVMGYEPEQIVIEMARENQTTNQGRRNSRQRYKLLEDGVKNLASDLNGNILKEYP |
| | TDNQALQNDRLFLYYLQNGKDMYTGEALDINQLSSYDIDHIIPQAFIKDDSLDNRVLTSSKDNRGK |
| | SDNVPSLEVVQKRKAFWQQLLDSKLISERKENNLTKAERGGLDERDKVGFIKRQLVETRQITKHVA |
| | QILDARFNTELDENGKRIRKVKIITLKSNLVSNFRKEFGLYKVREINDYHHAHDAYLNAVVAKAIL |
| | KKYPKLEPEFVYGDYQKYDLKRYISRSKDPKEVEKATEKYFFYSNLLNFFKEEVHYADGTIVKREN |
| | IEYSKDTGEIAWNKEKDFATVRKVLSCPQVNIVKKTEVQTGGFSKESILPKRNSDKLIARKKDWDP |
| | KKYGGFDSPTVAYSVLVVAKVEKGKSKKLKSVKELLGITIMERSSFEKNPIDFLEAKGYKEVRKDL |
| | IVKLPKYSLFELENGRKRMLASAGELQKGNELALPSKYVNFLYLASHYEKLKGSPEDNEQKQLFVE |
| | QHKHYLDEIIEQISEFSKRVILADANLDKVLSAYNKHRDKPIREQAENIIHLFTLTNLGAPAAFKYFD |
| | TTIDRKRYTSTKEVLDATFIHQSITGLYETRIDLSQLGGD |
| 185 | MTKPYSIGLDIGTNSVGWAVITDSYKVPSKKMKVLGNTSKKYVKKNLLGALLFESGITAEGRRLK |
| | RTARRRYTRRRNRILYLQEIFATEMAKIDESFFQRLDDSFLVPDDKRDSKYPIFGNLAQEKTYHDEF |
| | PTIYHLRKYLADSSKKADLRLVYLALAHMIKYRGHFLIEGDFNSRNNDIKKNFQNFLDSYNAIFESD |
| | LSLKNSEQLEEIVKDKISKSAKRERVLKLFPDEKSTGLFSEFLKLIVGNQADFKKHFDLEEKAPLQFS |
| | KDTYDEDLENLLGQIGDDFADLFLVAKKLYDAILLSGILTVTDPSTKAPLSASMIERYENHQKDLA |
| | ALKQFIQNNLQEKYDEVFSDESKDGYAGYIDGKTTQEAFYKYIKNLLSKLEGADYFLDKIEREDFL |
| | RKQRTFDNGSIPHQIHLQEMNAILRRQGEHYPFLQENKEKIEKILTFRIPYYVGPLARGNRDFAWLT |
| | RNSDQAIRPWNFEEVVDKASSAEDFINKMTNYDLYLPEEKVLPKHSLLYERFAVYNELTKVKFIAE |
| | GLRDYQFLDSGQKKQIVNQLFKEKRKVTEKDIIHYLHNVDGYDGIELKGIEKQFNASLSTYHDLLKI |
| | IKDKEFMDDPKNEEILENIIHTLTIFEDREMIKQRLAQYDSLFDEKVIKALTRRHYTGWGKLSAKLIN |
| | GIYDKQSKKTILDYLIDDDKINRNFMQLINDDGLSFKDIIQKAQVVGKTDDVKQVVQNLPGSPAIK |
| | KGILQSIKIVDELVKVMGSDPESIVIEMARENQTTNQGRRNSQQRYKKIEDAIKNLDSNLNSKILKE |
| | YPTDNQALQNDRLFLYYLQNGKDMYTGEPLDINQLSNYDIDHIVPQAFIKDDSLDNRVLTSSKDNR |
| | GKSDNVPSEEVVKRMRGFWRQLLDSKLISQRKFDNLTKAERGGLTEHDKAGFIKRQLVETRQITK |
| | HVAQILDARFNTEVDEKNKRIRNVKIITLKSNLVSNFRKEFRLYKVREINDYHHAHDAYLNAVVAK |
| | AILKKYPKLEPEFVYGDYQKYDIKRYIKRSKPHGTVEKATQKYFFYSNLLNFFKEEVHYADGTIVK |
| | REAVEYSKDTGEIAWNKEKDFATVRKVLSCPQVNIVKKTEVQTGGFSKESILPKRNSDKLIARKKD |
| | WDPKKYGGFDSPTVAYSVLVVAKVEKGKSKKLKSVKELLGITIMERSSFEKNPIDFLEAKGYKEVR |
| | KDLIVKLPKYSLFELENGRKRMLASAGELQKGNELALPSKYVNFLYLASHYEKLKGSPEDNEQKQ |
| | LFVEQHKHYLDEIIEQISEFSKRVILADANLDKVLSAYNKHRDKPIREQAENIIHLFTLTNLGAPAAF |
| | KYFDTTIDRKRYTSTKEVLDATFIHQSITGLYETRIDLSQLGGD |
| 186 | MTKPYSIGLDIGTNSVGWAVITDSYKVPSKKMKVLGNTSKKYVKKNLLGALLFESGITAEGRRLK |
| | RTARRRYTRRRNRILYLQEIFSTEMAKIDESFFQRLDDSFLVPDDKRDSKYPIFGNLVEEKTYHDEFP |
| | TIYHLRKYLADSTKKADLRLVYLALAHMIKYRGHFLIEGDFNSRNNDIKKNFQDFLDSYNAIFESD |
| | LSLENSKQLEEIVKDKISKSAKKDRLLKLFPGEKNSGIFSEFLKLIVGNQADFRKYFNLDEKASLHES |
| | KESYDEDLETLLGHIGDDYSDVFLKAKKLYDAILLSGILTVTDNETEAPLSSAMIMRYKEHEEDLAL |
| | LKAYIRKISLETYNEVFKDDTKNGYAGYIDGKTNQEDFYVYLKKLLTGLEGADYFLEKINREDLLR |
| | KQRTFDNGTIPYQIHLQEMRAILDKQAKFYPFLAKNKEKIEKILTFRIPYYVGPLARGNSDFAWSIR |
| | KRNEKITPWNFEDVIDKESSAEAFINRMTSFDLYLPEEKVLPKHSLLYETFTVYNELTKVKFIAEGM |
| | RDYQFLDSKQKKDIVRLYFKGKRKVTDKDIIEYLHAIDGYDGIELKGIEKQFNSSLATYHDLLNIIN |
| | DKEFLDDSSNEAIIEEIIHTLTIFEDREMIKQRLSKFENIFDKSVLKKLSRRHYTGWGKLSAKLINGIR |
| | DEKSGKTILDYLIDDGVSNRNFMQLIHDDTLSFKKKIQKAQIIGDQDNIKQVVQSLPGSPAIKKGILQ |
| | SIKIVDELVKVMGRRPESIVVEMARENQYTNQGKSNSQQRLKRLEESLKELGSKILKENVPVKLSKI |
| | DNQALQNDRLYLYYLQNGKDMYTGEELDIDRLSNYDIDHIIPQSFLTDNSIDNKVLVSSASNRGKS |
| | DDVPSLEVVKKRKGFWHQLLKSKLISKRKFDNLTKAERGGLTEADKAGFIKRQLVETRQITKHVA |
| | QILDARFNTEKDEDNKAIRTVKVITLKSNLVSQFRKDFELYKVREINDFHHAHDAYLNAVVATAIL |
| | KKYPKLEPEFVYGEYKKYDIRKFITNSSDKATAKYFFYSNLMNFFKRVIRYANGKVIVRPVVEYSK |
| | DTGEIAWNKEKDFATVRKVLSCPQVNIVKKTEVQTGGFSKESILPKRNSDKLIARKKDWDPKKYG |
| | GFDSPTVAYSVLVVAKVEKGKSKKLKSVKELLGITIMERSSFEKNPIDFLEAKGYKEVRKDLIVKLP |
| | KYSLFELENGRKRMLASAGELQKGNELALPSKYVNFLYLASHYEKLKGSPEDNEQKQLFVEQHKH |
| | YLDEIIEQISEFSKRVILADANLDKVLSAYNKHRDKPIREQAENIIHLFTLTNLGAPAAFKYFDTTIDR |
| | KRYTSTKEVLDATFIHQSITGLYETRIDLSQLGGD |
| 187 | MTKPYSIGLDIGTNSVGWAVITDSYKVPSKKMKVLGNTSKKYVKKNLLGALLFESGITAEGRRLK |
| | RTARRRYTRRRNRILYLQEIFSTEMAKVDENFFQRLDDSFLVPDDKRDSKYPIFGNLVEEKAYHDE |
| | FPTIYHLRKYLADSSKKADLRLVYLALAHMIKYRGHFLIEGDFNSRNNDIKKNFQDFLDSYNAIFES |
| | DLSLENSKQLEEIVKDKISKSAKRERVLKLFPDEKSTGLFSEFLKLIVGNQADFKKHFDLEEKAPLQF |
| | SKDTYDEDLENLLGQIGDDFADLFLVAKKLYDAILLSGILTVTDPSTKAPLSASMIERYENHQKDLA |
| | ALKQFVKANLPEKYDEVFSDQSKDGYAGYIDGKTTQEAFYKYIKNLLSKFEGADYFLEKIEREDFL |
| | RKQRTFDNGSIPHQIHLQEMNAIIRRQGEYYPFLKENKEKIEKILTFRIPYYVGPLARGNRDFAWLTR |
| | NSDQAIRPWNFEEIVDQASSAEEFINKMTNYDLYLPEEKVLPKHSLLYETFAVYNELTKVKFIAEGL |
| | RDYQFLDSGQKKQIVNQLFKEKRKVTEKDIIQFLHNVDGYDGIELKGIEKQFNASLSTYHDLLKIIK |
| | DKEFMDDPKNEEILENIVHTLTIFEDREMIKQRLAQYDSLFDEKVIKALARRHYTGWGKLSAKLIN |
| | GIYDKQSKKTILDYLIDDDKINRNFMQLINDDGLSFKEIIQKAQVVGKTDDVKQVVQELPGSPAIKK |
| | GILQSIKIVDELVKVMGHAPESIVIEMARENQTTNQGRRNSQQRYKKIQEAIKNLAPDLDGNILKEY |
| | PTDNQALQNDRLFLYYLQNGKDMYTGESLDINQLSSYDIDHIVPQAFIKDDSLDNRVLTSSKDNRG |
| | KSDNVPSLEVVQKRKAFWQQLLDSKLISERKENNLTKAERGGLNERDKVGFIKRQLVETRQITKHV |
| | AQILDARFNTEVNEEGKRIRKVKIITLKSNLVSNFRKEFGLYKVREINDYHHAHDAYLNAVVAKAI |
| | LKKYPKLEPEFVYGDYQKYDIKRYISRSKNPKEIDKATEKYFFYSNLLNFFKEEVHYADGTIVKREN |
| | IEYSNDTGEIAWNKEKDFATVRKVLSCPQVNIVKKTEVQTGGFSKESILPKRNSDKLIARKKDWDP |
| | KKYGGFDSPTVAYSVLVVAKVEKGKSKKLKSVKELLGITIMERSSFEKNPIDFLEAKGYKEVRKDL |
| | IVKLPKYSLFELENGRKRMLASAGELQKGNELALPSKYVNFLYLASHYEKLKGSPEDNEQKQLFVE |
| | QHKHYLDEIIEQISEFSKRVILADANLDKVLSAYNKHRDKPIREQAENIIHLFTLTNLGAPAAFKYFD |
| | TTIDRKRYTSTKEVLDATFIHQSITGLYETRIDLSQLGGD |
| 188 | MTKPYSIGLDIGTNSVGWAVITDSYKVPSKKMKVLGNTSKKYVKKNLLGALLFESGITAEGRRLK |
| | RTARRRYTRRRNRILYLQEIFSTEMAKIDESFFQRLDDSFLVPDDKRDSKYPIFGNLAQEKTYHDEF |
| | PTIYHLRKYLADSSKKADLRLVYLALAHMIKYRGHFLIEGDFNSRNNDIKKNFQDFLDSYNAIFESD |
| | LSLENSKQLEEIVKDKISKSAKRERVLKLFPDEKSTGLFSEFLKLIVGNQADFKKHFDLEEKAPLQFS |
| | KDTYDEDLENLLGQIGDDFADLFLVAKKLYDAILLSGILTVTDPSTKAPLSASMIERYENHQKDLAS |
| | LKQFIQNNLQEKYDEVFSDQSKDGYAGYIDGKTTQEAFYKYIKNLLSKFEGSDYFLDKIEREDFLR |
| | KQRTFDNGSIPHQIHLQEMNAILRRQGEHYPFLKENKEKIEQILTFRIPYYVGPLARGNRDFAWLTR |
| | NSDQAIRPWNFEEVVDQASSAEDFINKMTNYDLYLPEEKVLPKHSLLYETFAVYNELTKVKFIAEG |
| | LRDYQFLDSGQKKQIVNQLFKEKRKVTEKDIIQFLHNVDGYDGIELKGIEKQFNASLSTYHDLLKII |
| | KDKDFMDDPKNEEILENIVHTLTIFEDREMIKQRLAQYDSIFDEKVIKALTRRHYTGWGKLSAKLIN |
| | GIYDKQSKKTILDYLIDDDKINRNFMQLINDDGLSFKEIIQKAQVVGKTDDVKQVVQELPGSPAIKK |
| | GILQSIKIVDELVKVMGHAPESIVIEMARENQTTNQGRRNSQQRYKLIEDGVKNLASDLNGNILKEY |
| | PTNNQALQNERLFLYYLQNGKDMYTGEALDINQLSSYDIDHIIPQAFIKDDSLDNRVLTSSKDNRG |
| | KSDNVPSIEVVQKRKAFWQQLLDSKLISERKENNLTKAERGGLDERDKVGFIKRQLVETRQITKHV |
| | AQILDARFNTEVNENNQKIRKVKIITLKSNLVSNFRKEFGLYKVREINDYHHAHDAYLNAVVAKAI |
| | LKKYPKLEPEFVYGDYQKYDLKRYISKSKDPKDIEKATEKYFFYSNLLNFFKEEVHYADGTIIKREN |
| | VEYSNDTGEIAWNKEKDFATVRKVLSCPQVNIVKKTEVQTGGFSKESILPKRNSDKLIARKKDWDP |
| | KKYGGFDSPTVAYSVLVVAKVEKGKSKKLKSVKELLGITIMERSSFEKNPIDFLEAKGYKEVRKDL |
| | IVKLPKYSLFELENGRKRMLASAGELQKGNELALPSKYVNFLYLASHYEKLKGSPEDNEQKQLFVE |
| | QHKHYLDEIIEQISEFSKRVILADANLDKVLSAYNKHRDKPIREQAENIIHLFTLTNLGAPAAFKYFD |
| | TTIDRKRYTSTKEVLDATFIHQSITGLYETRIDLSQLGGD |
| 189 | MNKPYSIGLDIGTNSVGWAVVTDSYKVPSKKMKVLGNTSKKYVKKNLLGALLFESGITAEGRRLK |
| | RTARRRYTRRRNRILYLQEIFSEEMNKIDDSFFQRLDDSFLVVEDKRGERHPIFGNLAQEKTYHEKF |
| | PTIYHLRKYLADSSKKADLRLVYLALAHMIKYRGHFLIEGDFNSRNNDIKGTFQKFLDSYNAIFESD |
| | LSLKNSEQLEEIVKDKISKSAKRERVLKLFPDEKSTGLFSEFLKLIVGNQADFKKHFDLEEKAPLQFS |
| | KDTYDEDLENLLGQIGDGFADLFLVAKKLYDAILLSGILTVTDPSTKAPLSASMIERYENHQKDLA |
| | ALKQFIQNNLQEKYDEVFSDQSKDGYAGYIDGKTTQEAFYKYIKNLLSKLEGADYFLDKIEREDFL |
| | RKQRTFDNGSIPHQIHLQEMNAIIRRQGEHYPFLQENKEKIEKILTFRIPYYVGPLARGNRDFAWLTR |
| | NSDQAIRPWNFEEIVDKASSAEDFINKMTNYDLYLPEEKVLPKHSLLYERFAVYNELTKVKFIAEGL |
| | RDYQFLDSGQKKQIVNQLFKEKRKVTEKDIIHYLHNVDGYDGIELKGIEKQFNASLSTYHDLLKIIK |
| | DKEFMDDPKNEEILENIVHTLTIFEDREMIKQRLAQYDSLFDEKVIKALTRRHYTGWGKLSAKLING |
| | ICDKQTGNTILDYLIDDDKINRNFMQLINDDGLSFKDIIQKAQVVGKTDDVKQVVQELPGSPAIKKG |
| | ILQSIKIVDELVKVMGHAPESIVIEMARENQTTNQGKRNSQQRYKRLEEGIKNLAKDLNGNILKEYP |
| | TDNQALQNDRLFLYYLQNGKDMYTGEPLDINQLSSYDIDHIIPQAFIKDDSLDNRVLTSSKDNRGK |
| | SDNVPSLEVVQKRKAFWQQLLDSKLISERKENNLTKAERGGLNELDKVGFIKRQLVETRQITKHVA |
| | QILDERFNNEVDSKGKRIRKVKIITLKSNLVSNFRKEFGLYKVREINDYHHAHDAYLNAVVAKAIL |
| | KKYPKLEPEFVYGDYQKYDIKRYISRSKDPKEIEKATEKYFFYSNLLNFFKEEVHYADGTIIKRENIE |
| | YSKDTGEIAWNKEKDFATVRKVLSCPQVNIVKKTEVQTGGFSKESILPKRNSDKLIARKKDWDPK |
| | KYGGFDSPTVAYSVLVVAKVEKGKSKKLKSVKELLGITIMERSSFEKNPIDFLEAKGYKEVRKDLI |
| | VKLPKYSLFELENGRKRMLASAGELQKGNELALPSKYVNFLYLASHYEKLKGSPEDNEQKQLFVE |
| | QHKHYLDEIIEQISEFSKRVILADANLDKVLSAYNKHRDKPIREQAENIIHLFTLTNLGAPAAFKYFD |
| | TTIDRKRYTSTKEVLDATFIHQSITGLYETRIDLSQLGGD |
| 190 | MTKPYSIGLDIGTNSVGWAVISDSYKVPSKKMKVLGNTSKKYVKKNLLGALLFESGITAEGRRLKR |
| | TARRRYTRRRNRILYLQEIFSTEMAKIDESFFQRLDDSFLVPDDKRDSKYPIFGNLVEEKTYHDEFPT |
| | IYHLRKYLADSSKKADLRLVYLALAHMIKYRGHFLIEGDFNSRNNDIKKNFQDFLDSYNAIFESDLS |
| | LENSKQLEEIVKDKISKSAKRERVLKLFPDEKSTGLFSEFLKLIVGNQADFKKHFDLEEKAPLQFSK |
| | DTYDEDLENLLGQIGDGFADLFLVAKKLYDAILLSGILTVTDPSTKAPLSASMIERYENHQKDLATL |
| | KQFIKNNLSEKYDEVFSDQSKDGYAGYIDGKTTQEAFYKYIKNLLSKFEGSDYFLDKIEREDFLRK |
| | QRTFDNGSIPHQIHLQEMNAIIRRQGEHYPFLKENKEKIEKILTFRIPYYVGPLARGNRDFAWLTRNS |
| | DQAIRPWNFEEVVDKASSAEDFINKMTNYDLYLPEEKVLPKHSLLYETFAVYNELTKVKFIAEGLR |
| | DYQFLDSGQKKQIVNQLFKEKRKVTEKDIIHYLHNVDGYDGIELKGIEKQFNASLSTYHDLLKIIKD |
| | KEFMDDPKNEEILENIVHTLTIFEDREMIKQRLAQYDTLFDEKVIKALTRRHYTGWGKLSAKLINGI |
| | RDNRTRKTILDYLIDDGEINRNFMQLIHDDGLSFKEIIQKAQVVGKTDDVKQVVQELPGSPAIKKGI |
| | LQSIKLVDELVKVMGHAPESIVIEMARENQTTARGKKNSQQRYKRIEDALKNLAPGLDSNILKENP |
| | TDNIQLQNDRLFLYYLQNGRDMYTGEPLDINQLSNYDIDHIVPQAFIKDDSLDNRVLTSSKDNRGK |
| | SDNVPSIEVVQKRKGFWRQLLDSQLISQRKFDNLTKAERGKLTEKDKAGFIKRQLVETRQITKHVA |
| | QILDARFNTKRDENDKVIRDVKIITLKSNLVSNFRKEFGLYKVREINDYHHAHDAYLNAVVAKAIL |
| | KKYPKLEPEFVYGDYQKYDIKRYISRSKDPKDVEKATEKYFFYSNLLNFFKEEVHYADGTIIKRENI |
| | EYSKDTGEIAWNKEKDFATVRKVLSCPQVNIVKKTEVQTGGFSKESILPKRNSDKLIARKKDWDPK |
| | KYGGFDSPTVAYSVLVVAKVEKGKSKKLKSVKELLGITIMERSSFEKNPIDFLEAKGYKEVRKDLI |
| | VKLPKYSLFELENGRKRMLASAGELQKGNELALPSKYVNFLYLASHYEKLKGSPEDNEQKQLFVE |
| | QHKHYLDEIIEQISEFSKRVILADANLDKVLSAYNKHRDKPIREQAENIIHLFTLTNLGAPAAFKYFD |
| | TTIDRKRYTSTKEVLDATFIHQSITGLYETRIDLSQLGGD |
| 191 | MKKSYSIGLDIGTNSVGWAVVDDDYKVPAKKMRVFGDTDRSHIKKNLLGTLLFDSGETAESRRLK |
| | RTARRRYTRRRNRILYLQEIFAEEMAKVDDSFFHRLDESFLTDDDKTFDSHPIFGNKAEEDAYHQK |
| | FPTIYHLRKHLADSTEKADLRLVYLALAHMIKFRGHFLIEGQLNAENTDVQTLFNDFVEVYDKTVE |
| | ESHLAEITVDALSILTEKVSKSRRLENLIKYYPTEKKNTLFGNLVALALGLQPNFKTNFKLSEDAKL |
| | QFSKDTYEEDLEELLGKIGDEYADLFTSAKNLYDAILLSGILTVDDNSTKAPLSASMIKRYEEHHED |
| | LEKLKTFVKANNSDKYHEIFSDKDKNGYAGYIENGVKQDEFYKYLKNTLSKIAGSDYFLDKIERED |
| | FLRKQRTFDNGSIPHQIHLQEMHAILRRQGDYYPFLKENQDRIEKILTFRIPYYVGPLARKDSRFAW |
| | ANYHSDEPITPWNFDEVVDKEKSAEKFITRMTLNDLYLPEEKVLPKHSHVYETFAVYNELTKIKYV |
| | NEQGKSFFFDANMKQEIFDHVFKENRKVTKDKLLDFLNKEFEEFRIVDLIGLDKENKSFNASLGTY |
| | HDLKKILDKSFLDDKTNEQIIEDIVLTLTLFEDRDMIRQRLQKYSDIFTKQQLKKLERRHYTGWGRL |
| | SYKLINGIRNKENNKTILDFLIDDGRANRNFMQLINDDSLSFKTIIQEAQVVGDVDDIEAVVHDLPG |
| | SPAIKKGILQSVKIVDELVKVMGHNPDNIVIEMARENQTTNRGRVQSQQRLKKLQESLKELGSEILN |
| | VEKPSYVEGKVENNHLQNDKLFLYYIQNGKDMYTGEELDIDHLSDYDIDHIIPQAFIKDDSIDNRVL |
| | TSSAKNRGKSDDVPSLEIVHDRKADWIRLYKSGLISKRKFDNLTKAERGGLTEADKAGFIKRQLVE |
| | TRQITKHVAQILDARFNTERDENDKVIRDVKVITLKSNLVSQFRKDFKFYKVREINDYHHAHDAYL |
| | NAVVGTALLKKYPKLAPEFVYGEYKKYDIRKFITNSSDKATAKYFFYSNLLNFFKTKVRYADGTIL |
| | VRPVIEYSKDTGEIAWNKEKDFATVRKVLSCPQVNIVKKTEVQTGGFSKESILPKRNSDKLIARKKD |
| | WDPKKYGGFDSPTVAYSVLVVAKVEKGKSKKLKSVKELLGITIMERSSFEKNPIDFLEAKGYKEVR |
| | KDLIVKLPKYSLFELENGRKRMLASAGELQKGNELALPSKYVNFLYLASHYEKLKGSPEDNEQKQ |
| | LFVEQHKHYLDEIIEQISEFSKRVILADANLDKVLSAYNKHRDKPIREQAENIIHLFTLTNLGAPAAF |
| | KYFDTTIDRKRYTSTKEVLDATFIHQSITGLYETRIDLSQLGGD |
| 192 | MTKPYSIGLDIGTNSVGWAVITDSYKVPSKKMKVLGNTSKKYVKKNLLGALLFESGITAEGRRLK |
| | RTARRRYTRRRNRILYLQEIFATEMNKVDESFFHRLDDSFLVPEDKRGERHPIFGNLEEEKAYHEKF |
| | PTIYHLRKYLADSSKKADLRLVYLALAHMIKYRGHFLIEGDFNSRNNDIKKNFQEFLDSYNAIFESD |
| | LSLENSAQVEEIVKDKISKSAKRERVLKLFPDEKSTGLFSEFLKLIVGNQADFKKHFDLEEKAPLQFS |
| | KDTYDEDLENLLGQIGDDFADLFLVAKKLYDAILLSGILTVTDPSTKAPLSASMIERYENHQKDLA |
| | ALKQFVKTNLPEKYDEVFSDQSKDGYAGYINGKTTQEAFYKYIKNLLSKLEGADYFLDKIEREDFL |
| | RKQRTFDNGSIPHQIHLQEMNAILRRQGEHYPFLKENKEKIEKILTFRIPYYVGPLARGNRDFAWLT |
| | RNSDQAIRPWNFEEVVDKASSAEDFINKMTNYDLYLPEEKVLPKHSLLYETFAVYNELTKVKFIAE |
| | GLRDYQFLDSGQKKQIVNQLFKEKRKVTEKDIIHYLHNVDGYDGIELKGIEKQFNASLSTYHDLLKI |
| | IKDKEFMDDPKNEEILENIVHTLTIFEDREMIKQRLAQYDSLFDEKVIKALTRRHYTGWGKLSAKLI |
| | NGIRDKKSGKTILDYLIDDGEINRNFMQLIHDDGLSFKEIIQKAQVFGKTDDVKQVVQELPGSPAIK |
| | KGILQSIKIVDELVKVMGHAPESIVIEMARENQTTNQGKRNSQQRYKRLEDGIKNLAKDLNGKILK |
| | EYPTDNQALQNERLFLYYLQNGKDMYTDEELDINQLSNYDIDHIIPQAFIKDDSLDNRVLTSSKDN |
| | RGKSDNVPSIEVVEKMKAFWKQLLDSKLISERKYNNLTKAERERDGLNELDKVGFIKRQLVETRQI |
| | TKHVAQILDARFNTEVNEKGKKIRKVKIITLKSNLVSNFRKEFGLYKVREINDYHHAHDAYLNAVL |
| | AKAILKKYPKLEPEFVYGDYQKYDIKRYISKSKDPKDIDKATEKYFFYSNLLNFFKEEVHYADGTIV |
| | KRENIEYSKDTGEIAWNKEKDFATVRKVLSCPQVNIVKKTEVQTGGFSKESILPKRNSDKLIARKK |
| | DWDPKKYGGFDSPTVAYSVLVVAKVEKGKSKKLKSVKELLGITIMERSSFEKNPIDFLEAKGYKEV |
| | RKDLIVKLPKYSLFELENGRKRMLASAGELQKGNELALPSKYVNFLYLASHYEKLKGSPEDNEQK |
| | QLFVEQHKHYLDEIIEQISEFSKRVILADANLDKVLSAYNKHRDKPIREQAENIIHLFTLTNLGAPAA |
| | FKYFDTTIDRKRYTSTKEVLDATFIHQSITGLYETRIDLSQLGGD |
| 193 | MTKPYSIGLDIGTNSVGWAVITDSYKVPSKKMKVLGNTSKKYVKKNLLGALLFESGITAEGRRLK |
| | RTARRRYTRRRNRILYLQEIFSTEMSKIDESFFQRLDDSFLVPDDKRDSKYPIFGNLVEEKTYHDEFP |
| | TIYHLRKYLADSSKKADLRLVYLALAHMIKYRGHFLIEGEFNSKNNDIKKNFQEFLDSYNAIFESDL |
| | SLENSAQVEEIVKDKISKSAKRERVLKLFPDEKSTGLFSEFLKLIVGNQADFKKHFELEEKAPLQFSK |
| | DTYDEDLENLLGQIGDDFADLFLVAKKLYDAILLSGILTVTDPSTKAPLSASMIERYENHQKDLAAL |
| | KQFIKANLPEKYDEVFSDQSKDGYAGYINGKTTQEAFYKYIKNLLSKLEGADYFLDKIEREDFLRK |
| | QRTFDNGSIPHQIHLQEMNAIIRRQGEHYPFLKENKEKIQQILTFRIPYYVGPLARGNRDFAWLTRNS |
| | DQAIRPWNFEEIVDKASSAEEFINKMTNYDLYLPEEKVLPKHSLLYETFAVYNELTKVKFIAEGLRD |
| | YQFLDSGQKKQIVNQLFKDKRKVTEKDIIHYLHTVDGYDGIELKGIEKQFNASLSTYHDLLKIIKDK |
| | EFMDDPKNEEILENIVHTLTIFEDREMIKQRLAQYASIFDEKVIKALTRRHYTGWGKLSAKLINGIY |
| | DKQSKKTILDYLIDDGEINRNFMQLIHDDGLSFKEIIQKAQVVGKTDDLKQVVQELPGSPAIKKGIL |
| | QSIKIVDELVKVMGHAPESIVIEMARENQTTNQGKRNSQQRYKRIEDAIKNLSIDLNSKILKEYPTD |
| | NQALQNDRLFLYYLQNGKDMYTGEALDINQLSNYDIDHIIPQAFIKDDSLDNRVLTSSKDNRGKSD |
| | NVPSLEIVEKMKGFWQQLLDSKLISERKENNLTKAERGGLDERDKVGFIKRQLVETRQITKHVAQI |
| | LDARFNTEVNEKKQKIRTVKIITLKSNLVSNFRKEFGLYKVREINDYHHAHDAYLNAVVAKAILKK |
| | YPKLEPEFVYGDYQKYDIKRYISRSKDPKEVEKATQKYFFYSNLLNFFKEEVHYADGTIIKRENIEY |
| | SKDTGEIAWNKEKDFATVRKVLSCPQVNIVKKTEVQTGGFSKESILPKRNSDKLIARKKDWDPKK |
| | YGGFDSPTVAYSVLVVAKVEKGKSKKLKSVKELLGITIMERSSFEKNPIDFLEAKGYKEVRKDLIV |
| | KLPKYSLFELENGRKRMLASAGELQKGNELALPSKYVNFLYLASHYEKLKGSPEDNEQKQLFVEQ |
| | HKHYLDEIIEQISEFSKRVILADANLDKVLSAYNKHRDKPIREQAENIIHLFTLTNLGAPAAFKYFDT |
| | TIDRKRYTSTKEVLDATFIHQSITGLYETRIDLSQLGGD |
| 194 | MNKPYSIGLDIGTNSVGWAVITDDYKVPSKKMKVLGNTSKKYVKKNLLGALLFESGITAEGRRLK |
| | RTARRRYTRRRNRILYLQEIFSSEMDKIDESFFQRLDDSFLVPDDKRDSKYPIFGNLVEEKTYHDEFP |
| | TIYHLRKYLADSSKKADLRLVYLALAHMIKYRGHFLIEGDFNSRNNDIKKNFQDFLDSYNAIFESDL |
| | SLENSKQLEEIVKDKISKSAKRERVLKLFPDEKSTGLFSEFLKLIVGNQADFKKHFDLEEKAPLQFSK |
| | DTYDEDLENLLGQIGDDFADLFLVAKKLYDAILLSGILTVTDPSTKAPLSASMIERYENHQKDLAQL |
| | KQFVRRNIPEKYDEVFADQSKNGYAGYIDGKTTQEEFYKYIKNALNKIEGADYFLDKIEREDFLRK |
| | QRTFDNGSIPHQIHLQEMNAIIRRQGEHYPFLKENKEKIEKILTFRIPYYVGPLARGNRDFAWLTRNS |
| | DQAIRPWNFEEVVDQASSAEDFINKMTNYDLYLPEEKVLPKHSLLYETFAVYNELTKVKFIAEGLR |
| | DYQFLDSGQKKQIVNQLFKEKRKVTEKDIIQFLHNVDGYDGIELKGIEKQFNASLSTYHDLLKIIKD |
| | KEFMDDPKNEEILENIVHTLTIFEDREMIKQRLAQYDSLFDEKVIKALTRRHYTGWGKLSAKLINGI |
| | RDKQSQKTILDYLIDDDKINRNFMQLINDDGLSFKEIIQKAQVVGKTDDVKQVVQELPGSPAIKKGI |
| | LQSIKIVDELVKVMGHAPESIVIEMARENQTTARGKKNSQQRYKRIEDSLKNLAIGLDSKILKEHPT |
| | DNQALQNDRLFLYYLQNGKDMYTGKELDINQLSNYDIDHIIPQAFIKDDSLDNRVLTSSKDNRGKS |
| | DDVPSIEIVKARKGFWQQLLNSKLISERKENNLTKAERGGLDERDKVGFIKRQLVETRQITKHVAQI |
| | LDARFNTEVDENGKRIRKVKIITLKSNLVSNFRKEFGLYKVREINDYHHAHDAYLNAVVAKAILKK |
| | YPKLEPEFVYGDYQKYDIKRYISRSKDPKDIEKATEKYFFYSNLLNFFKEEVHYADGTILKRENIEY |
| | SNDTGEIAWNKEKDFATVRKVLSCPQVNIVKKTEVQTGGFSKESILPKRNSDKLIARKKDWDPKK |
| | YGGFDSPTVAYSVLVVAKVEKGKSKKLKSVKELLGITIMERSSFEKNPIDFLEAKGYKEVRKDLIV |
| | KLPKYSLFELENGRKRMLASAGELQKGNELALPSKYVNFLYLASHYEKLKGSPEDNEQKQLFVEQ |
| | HKHYLDEIIEQISEFSKRVILADANLDKVLSAYNKHRDKPIREQAENIIHLFTLTNLGAPAAFKYFDT |
| | TIDRKRYTSTKEVLDATFIHQSITGLYETRIDLSQLGGD |
| 195 | MKKYSIGLDIGTNSVGWAVVDDDYKVPAKKMRVFGDTDRSHIKKNLLGTLLFDSGETAESRRLKR |
| | TARRRYTRRRNRILYLQEIFAKEMAKVDESFFHRLDESFLTDDDKTFDSHPIFGNKAEEDAYHQKFP |
| | TIYHLRKHLADSTEKADLRLVYLALAHMIKFRGHFLIEGELNAENTDVQKLFAQFVGVYDRTFDDS |
| | HLSEITVDASSILTEKISKSRRLENLIKHYPTEKKNTLFGNLVALALGLQPNFKTNFKLSEDAKLQFS |
| | KDTYEEDLEELLGKIGDDYADLFTSAKNLYDAILLSGILTVDDESTKAPLSASMIKRYAEHHEDLEK |
| | LKEFIKANKPELYHDIFKDETKNGYAGYIENGVKQDEFYKYLKNTLSKIEGSDYFLDKIEREDFLRK |
| | QRTFDNGSIPHQIHLQEMHAILRRQGDYYPFLKENQDRIEKILTFRIPYYVGPLARKDSRFAWANYH |
| | SDEPITPWNFDEVVDKEKSAEKFITRMTLNDLYLPEEKVLPKHSPVYETYTVYNELTKVKYVTEQG |
| | KATFFDANMKQEIFEHVFKENRKVTKDKFLNYLNKEFPEYRIQDLIGLDKENKSFNASLGTYHDLK |
| | KILDKSFLDDKTNEEIIEDIIQTLTLFEDRDMIRQRLQKYSDIFTKQQLKKLERRHYTGWGRLSYKLI |
| | NGIRNKENNKTILDYLIDDGHANRNFMQLINDESLSFKTIIQEAQVVGDVDDIKAVVHDLPGSPAIK |
| | KGILQSVKIVDELVKVMGDNPDNIVIEMARENQTTNRGRTQSQQRLKKLQDSLKELGSNILNEEKP |
| | SYVEGKVENNHLQNDKLFLYYIQNGKDMYTGEELDIDHLSDYDIDHIIPQAFIKDDSIDNRVLTSSA |
| | KNRGKSDDVPSLDIVHDRKADWIRLYKSGLISKRKFDNLTKAERGGLTEADKAGFIKRQLVETRQI |
| | TKHVAQILDARFNTERDENDKVIRDVKVITLKSNLVSQFRKDFQFYKVREINDYHHAHDAYLNAV |
| | VGTALLKKYPKLAPEFVYGEYKKYDIRKFITASGDKATAKYFFYSNLLNFFKTKVRYADGTVIVRP |
| | VIEYSKDTGEIAWNKEKDFATVRKVLSCPQVNIVKKTEVQTGGFSKESILPKRNSDKLIARKKDWD |
| | PKKYGGFDSPTVAYSVLVVAKVEKGKSKKLKSVKELLGITIMERSSFEKNPIDFLEAKGYKEVRKD |
| | LIVKLPKYSLFELENGRKRMLASAGELQKGNELALPSKYVNFLYLASHYEKLKGSPEDNEQKQLFV |
| | EQHKHYLDEIIEQISEFSKRVILADANLDKVLSAYNKHRDKPIREQAENIIHLFTLTNLGAPAAFKYF |
| | DTTIDRKRYTSTKEVLDATFIHQSITGLYETRIDLSQLGGD |
| 196 | MTKPYSIGLDIGTNSVGWAVITDSYKVPSKKMKVLGNTSKKYVKKNLLGALLFESGITAEGRRLK |
| | RTARRRYTRRRNRILYLQEIFSTEMAKIDESFFQRLDDSFLVPDDKRDSKYPIFGNLVKEKTYHDEF |
| | PTIYHLRKYLADSSKKADLRLVYLALAHMIKYRGHFLIEGDFNSRNNDIKKNFQDFLDSYNAIFESD |
| | LSLENSKQLEEIVKDKISKSAKKDRLLKLFPGEKNSGIFSEFLKLIVGNQADFRKYFNLDEKASLHES |
| | KESYDEDLETLLGHIGDDYSDVFLKAKKLYDAILLSGILTVTDNETEAPLSSAMIMRYKEHEEDLAL |
| | LKAYIRKISLETYNEVFKDDTKNGYAGYIDGKTNQEDFYVYLKKLLTGLEGADYFLEKINREDLLR |
| | KQRTFDNGSIPYQIHLQEMRAIIDKQAKFYPFLAKNKERIEKILTFRIPYYVGPLARGNSDFAWSIRK |
| | RNEKITPWNFEDVIDKESSAEAFINRMTSFDLYLPEEKVLPKHSLLYETFTVYNELTKVRFIAESMR |
| | DYQFLDSKQKKDIVRLYFKGKRKVTDKDIIEYLHAIDGYDGIELKGIEKQFNSSLSTYHDLLNIINDK |
| | EFLDDSSNEAIIEEIIHTLTIFEDREMIKQRLSKFENIFDKSVLKKLSRRHYTGWGKLSAKLINGIRDE |
| | KSGKTILDYLIDDGVSNRNFMQLIHDDTLSFKKKIQKAQIIGDQDNIKQVVQSLPGSPAIKKGILQSI |
| | KIVDELVKVMGREPESIVVEMARENQYTNQGKSNSQQRLKRLEESLKELGSKILKENVPAKLSKID |
| | NNALQNERLYLYYLQNGKDMYTGDDLDIDRLSNYDIDHIIPQAFLKDNSIDNKVLVSSASNRGKSD |
| | DVPSIEVVKARKTFWHQLLKSQLISQRKFDNLTKAERGGLTDDDKAGFIKRQLVETRQITKHVAQL |
| | LDSRFNTKRDDNDKVIREVKIITLKSSLVSQFRKDFKLYKVREINDYHHAHDAYLNAVVATAILKK |
| | YPKLEPEFVYGDYQKYDIRKYLSTKDNKDLGKATAKMFFYSNLMNFFKKEVRLADGTVVVRPVIE |
| | YSKDTGEIAWNKEKDFATVRKVLSCPQVNIVKKTEVQTGGFSKESILPKRNSDKLIARKKDWDPK |
| | KYGGFDSPTVAYSVLVVAKVEKGKSKKLKSVKELLGITIMERSSFEKNPIDFLEAKGYKEVRKDLI |
| | VKLPKYSLFELENGRKRMLASAGELQKGNELALPSKYVNFLYLASHYEKLKGSPEDNEQKQLFVE |
| | QHKHYLDEIIEQISEFSKRVILADANLDKVLSAYNKHRDKPIREQAENIIHLFTLTNLGAPAAFKYFD |
| | TTIDRKRYTSTKEVLDATFIHQSITGLYETRIDLSQLGGD |
| 197 | MTKPYSIGLDIGTNSVGWAVITDSYKVPSKKMKVLGNTSKKYVKKNLLGALLFESGITAEGRRLK |
| | RTARRRYTRRRNRILYLQEIFSTEMAKIDESFFQRLDDSFLVPDDKRDSKYPIFGNLVKEKTYHDEF |
| | PTIYHLRKYLADSSKKADLRLVYLALAHMIKYRGHFLIEGDFNSRNNDIKKNFQDFLDSYNAIFESD |
| | LSLENSKQLEEIVKDKISKSAKKDRLLKLFPGEKNSGIFSEFLKLIVGNQADFRKYFNLDEKASLHES |
| | KESYDEDLETLLGHIGDDYSDVFLKAKKLYDAILLSGILTVTDNETEAPLSSAMIMRYKEHEEDLAL |
| | LKAYIRNISLETYNEVFKDDTKNGYAGYIDGKTNQEDFYVYLKKLLTGLEGADYFLEKINREDLLR |
| | KQRTFDNGSIPYQIHLQEMRAIIDKQAKFYPFLAKNKERIEKILTFRIPYYVGPLARGNSDFAWSIRK |
| | RNEKITPWNFEDVIDKESSAEAFINRMTSFDLYLPEEKVLPKHSLLYETFTVYNELTKVRFIAESMR |
| | DYQFLDSKQKKDIVRLYFKGKRKVTDKDIIDYLHAIDGYDGIELKGIEKQFNSSLSTYHDLLNIIND |
| | KEFLDDSSNEAIIEEIIHTLTIFEDREMIKQRLSKFENIFDKSVLKKLSRRHYTGWGKLSAKLINGIRD |
| | EKSGKTILDYLIDDGVSNRNFMQLIHDDTLTFKKKIQKAQYIEDQDNIKQVVQSLPGSPAIKKGILQS |
| | IKIVDELVKVMGGRKPESIVVEMARENQYTNQGKSNSQQRLKRLEESLKELGSKILKENVPAKLSK |
| | IDNNALQNERLYLYYLQNGKDMYTGDDLDIDRLSNYDIDHIIPQAFLKDNSIDNKVLVSSASNRGK |
| | SDDVPSLDVVKKRKTFWHQLLKSQLISQRKFDNLTKAERGGLTDDDKAGFIKRQLVETRQITKHV |
| | AQLLDSRFNTKRDDNDKVIRDVKIITLKSSLVSQFRKEFGLYKVREINDYHHAHDAYLNAVVATAI |
| | LKKYPKLEPEFVYGEYKKYDVRKFIAKSDKSLGKATAKMFFYSNLMNFFKKEVRLADGTVVVRP |
| | VVEYSNDTGEIAWNKEKDFATVRKVLSCPQVNIVKKTEVQTGGFSKESILPKRNSDKLIARKKDW |
| | DPKKYGGFDSPTVAYSVLVVAKVEKGKSKKLKSVKELLGITIMERSSFEKNPIDFLEAKGYKEVRK |
| | DLIVKLPKYSLFELENGRKRMLASAGELQKGNELALPSKYVNFLYLASHYEKLKGSPEDNEQKQLF |
| | VEQHKHYLDEIIEQISEFSKRVILADANLDKVLSAYNKHRDKPIREQAENIIHLFTLTNLGAPAAFKY |
| | FDTTIDRKRYTSTKEVLDATFIHQSITGLYETRIDLSQLGGD |
| 198 | MKKYSIGLDIGTNSVGWAVVDDDYKVPAKKMRVFGDTDRSHIKKNLLGTLLFDSGETAESRRLKR |
| | TARRRYTRRRNRILYLQEIFAEEMSKVDDSFFHRLDESFLTDDDKNFDSHPIFGNKAEEDAYHQKFP |
| | TIYHLRKHLADSTEKADLRLVYLALAHMIKFRGHFLIEGQLNAENTDVQTLFNDFVEVYDKTVEES |
| | HLSEITVDALSILTEKVSKSRRLENLIKYYPTEKKNTLFGNLIALALGLQPNFKTNFKLSEDAKLQFS |
| | KDTYEEDLEELLGKIGDDYADLFTSAKNLYDAILLSGILTVDDNSTKAPLSASMIKRYVEHHEDLE |
| | KLKDFIRANKPDFYHDIFKDENKNGYAGYIENGVKQDEFYKYLKNTLSKIAGSDYFLDKIEREDFL |
| | RKQRTFDNGSIPHQIHLQEMHAILRRQGDYYPFLKENQDRIEKILTFRIPYYVGPLARKDSRFAWAE |
| | YHLDEKITPWNFDKVIDKEKSAEKFITRMTSNDLYLPEEKVLPKHSHVYETYAVYNELTKIKYVNE |
| | QGEESFFDANMKQEIFDHVFKENRKVTKEKLLNYLNKEFPEYRIQDLIGLDKENKSFNASLGTYHD |
| | LKKILDKSFLDDKTNEQIIEDIIQTLTLFEDRDMIRQRLQKYSDIFTKQQLKKLERRHYTGWGRLSY |
| | KLINGIRNKENNKTILDFLIDDGHANRNFMQLINDDSLSFKTIIQEAQVVGDVDDIEAVVHDLPGSP |
| | AIKKGILQSVKIVDELVKVMGHNPDNIVIEMARENQTTNRGRSQSQQRLKKLQESLKELGSNILNEE |
| | KPSYIEDKVENSQLQNDRLFLYYIQNGKDMYTGDELDIDHLSDYDIDHIIPQAFIKDDSIDNRVLTSS |
| | AKNRGKSDDVPSLDIVRDRKADWIRLYKSGLISKRKFDNLTKAERGGLTEADKAGFIKRQLVETRQ |
| | ITKHVAQILDARFNTKRDENDKVIRDVKVITLKSNLVSQFRKDFGFYKVREINDYHHAHDAYLNA |
| | VVGTALLKKYPKLAPEFVYGEYKKYDVRKLIVQSDQKYGKATAKMFFYSNLMNFFKKEVRLSDG |
| | TVIVRPVIEYSKDTGEIAWNKEKDFATVRKVLSCPQVNIVKKTEVQTGGFSKESILPKRNSDKLIAR |
| | KKDWDPKKYGGFDSPTVAYSVLVVAKVEKGKSKKLKSVKELLGITIMERSSFEKNPIDFLEAKGY |
| | KEVRKDLIVKLPKYSLFELENGRKRMLASAGELQKGNELALPSKYVNFLYLASHYEKLKGSPEDN |
| | EQKQLFVEQHKHYLDEIIEQISEFSKRVILADANLDKVLSAYNKHRDKPIREQAENIIHLFTLTNLGA |
| | PAAFKYFDTTIDRKRYTSTKEVLDATFIHQSITGLYETRIDLSQLGGD |
| 199 | MKKPYSIGLDIGTNSVGWAVVTDDYKVPAKKMKVLGNTDKSHIKKNLLGALLFDSGETAEGTRL |
| | KRTARRRYTRRRNRILYLQEIFAKEMAKVDESFFQRLEESFLTDDDKTFDSHPIFGNKAEEDVYHQ |
| | KFPTIYHLRKHLADSPEKADLRLVYLALAHMIKFRGHFLIEGQLNAENTDVQTLFNDFVEVYDKTV |
| | EESHLSEITVDALSILTEKVSKSRRLENLIKYYPTEKKNTLFGNLIALALGLQPNFKTNFKLSEDAKL |
| | QFSKDTYEEDLEELLGKIGDEYADLFTSAKNLYDAILLSGILTVDDNSTKAPLSASMLKRYVEHHE |
| | DLEKLKEFIKANKSELYHDIFKDKNKNGYAGYIENGVKQDEFYKYLKNTLSKIEGSDYFLDKIERE |
| | DFLRKQRTFDNGSIPHQIHLQEMHAILRRQGEYYPFLKENQAKIEKILTFRIPYYVGPLARKDSRFA |
| | WANYHSDEPITPWNFDEVVDKEKSAEKFITRMTLNDLYLPEEKVLPKHSHVYETYTVYNELTKIK |
| | YVNEQGKATFFDANMKQEIFEHVFKENRKVTKEKFLNYLNKEFPEYRIQDLIGLDKENKSFNASLG |
| | TYHDLKKILDKSFLDDKTNEQIIEDIIQTLTLFEDRDMIRQRLQKYSDIFTKQQLKKLERRHYTGWG |
| | RLSYKLINGIRNKENNKTILDYLIDDGYANRNFMQLINDDTLPFKQIIQEAQVVGDVDDIEAVVHDL |
| | PGSPAIKKGILQSVKIVDELVKVMGHNPDNIVIEMARENQTTNRGRSQSQQRLKKLQESLKELGSNI |
| | LNEEKPSYIEDKVENSHLQNDKLFLYYIQNGKDMYTGDELDIDHLSDYDIDHIIPQAFIKDDSIDNR |
| | VLTSSAKNRGKSDDVPSLDIVHDRKADWIRLYKSGLISKRKFDNLTKAERGGLTEADKAGFIKRQL |
| | VETRQITKHVAQILDSRFNTKRDENDKVIRDVKVITLKSNLVSQFRKDFQFYKVREINDYHHAHDA |
| | YLNAVVGTALLKKYPKLAPEFVYGEYKKYDIRKFISSSSDKATAKYFFYSNLLNFFKTKVRYADGT |
| | VIVRPVIEYSKDTGEIAWNKEKDFATVRKVLSCPQVNIVKKTEVQTGGFSKESILPKRNSDKLIARK |
| | KDWDPKKYGGFDSPTVAYSVLVVAKVEKGKSKKLKSVKELLGITIMERSSFEKNPIDFLEAKGYKE |
| | VRKDLIVKLPKYSLFELENGRKRMLASAGELQKGNELALPSKYVNFLYLASHYEKLKGSPEDNEQ |
| | KQLFVEQHKHYLDEIIEQISEFSKRVILADANLDKVLSAYNKHRDKPIREQAENIIHLFTLTNLGAPA |
| | AFKYFDTTIDRKRYTSTKEVLDATFIHQSITGLYETRIDLSQLGGD |
| 200 | MTKPYSIGLDIGTNSVGWAVITDSYKVPSKKMKVLGNTSKKYVKKNLLGALLFESGITAEGRRLK |
| | RTARRRYTRRRNRILYLQEIFSTEMAKIDESFFQRLDDSFLVPDDKRDSKYPIFGNLVEEKAYHDEF |
| | PTIYHLRKYLADSSKKADLRLVYLALAHMIKYRGHFLIEGEFNSKNNDIKKNFQEFLDSYNAIFESD |
| | LSLENSAQVEEIVKDKISKSAKRERVLSLFPDEKSTGLFSEFLKLIVGNQADFKKHFDLEEKAPLQFS |
| | KDTYDEDLENLLGQIGDDFADLFLVAKKLYDAILLSGILTVTDPSTKAPLSASMIERYENHQKDLA |
| | QLKQFVRRNLPEKYAEVFADQSKNGYAGYIDGKTTQEEFYKYLKEELTGVKGADYFLEKIKREDF |
| | LRKQRTFDNGSIPYQVHLTEMKAIIHRQGEHYPFLKENKEKIEKILTFRIPYYVGPLARGNRDFAWL |
| | TRNSDQAIRPWNFEEIVDKASSAEDFINKMTNYDLYLPEEKVLPKHSLLYETFAVYNELTKVKFIAE |
| | GLRDYQFLDSGQKKQIVNQLFKEKRKVTEKDIIHYLHNVDGYDGIELKGIEKQFNASLSTYHDLLKI |
| | IKDKEFMDDPKNEEILENIVHTLTIFEDREMIKQRLAQYDSLFDEKVIKALTRRHYTGWGKLSAKLI |
| | NGICDKQTGDTILDYLIDDDKINRNFMQLINDDGLSFKEIIQKAQVVGKTDDVKQVVQELPGSPAIK |
| | KGILQSIKIVDELVKVMGHAPESIVIEMARENQTTARGKKNSQQRYKRIEDSLKNLASGLDSNILKE |
| | NPTDNVQLQNDRLFLYYLQNGRDMYTGKPLDINQLSNYDIDHIIPQAFIKDDSLDNRVLTSSKDNR |
| | GKSDNVPSLEVVEKMKAFWQQLLDSKLISERKYNNLTKAERERGGLNELDKVGFIKRQLVETRQI |
| | TKHVAQILDARFNTEVDDKGKRIRKVKIITLKSNLVSNFRKEFGLYKVREINDYHHAHDAYLNAVV |
| | AKAILKKYPKLEPEFVYGDYQKYDIKRYISRSKDPKDIEKATEKYFFYSNLLNFFKEEVHYADGTII |
| | KRENIEYSKDTGEIAWNKEKDFATVRKVLSCPQVNIVKKTEVQTGGFSKESILPKRNSDKLIARKK |
| | DWDPKKYGGFDSPTVAYSVLVVAKVEKGKSKKLKSVKELLGITIMERSSFEKNPIDFLEAKGYKEV |
| | RKDLIVKLPKYSLFELENGRKRMLASAGELQKGNELALPSKYVNFLYLASHYEKLKGSPEDNEQK |
| | QLFVEQHKHYLDEIIEQISEFSKRVILADANLDKVLSAYNKHRDKPIREQAENIIHLFTLTNLGAPAA |
| | FKYFDTTIDRKRYTSTKEVLDATFIHQSITGLYETRIDLSQLGGD |
| 201 | MKKPYSIGLDIGTNSVGWAVITDDYKVPAKKMKVLGNTDRSHIKKNLIGALLFDAGNTAEDRRLK |
| | RTARRRYTRRRNRILYLQEIFAEEMNKIDESFFHRLDDSFLVPEDKRGSKYPIFATLQEEKEYHKQFP |
| | TIYHLRKQLAESNEKADLRLVYLALAHMIKYRGHFLIDDPKFKVQNNDIQGLFEKFVEEYDNVQET |
| | SLSKIKLNVTEILTAKIPKSEKQEQLLKNYPSEKKNTLFGNLIGLALGLTPNFKTNFSLENDAKLQISS |
| | ESYEEDLGSLLALIGENFIELFSAVKNLSDGILLAGIVSDESPHAPLSTKMVIRFKEHEEDLAALKHFI |
| | KANLPEKYDEVFSDDSKNGYAGYVGVDSKVRKRNGKLATEEEFYKYLKDILNNVKGADYFLEKI |
| | KREDLLRKQRTFDNGTIPYQVHLEEMKAILQNQGEYYPFLKENKEKIQQILTFRIPYYVGPLARGNR |
| | DFAWLTRNSDQAIRPWNFEEVVDKASSAEDFINKMTNYDLYLPEEKVLPKHSLLYETFAVYNELT |
| | KVKFIAEGLRDYQFLDSGQKKQIVNQLFKEKRKVTEKDIIHYLHNVDGYDGIELKGIEKQFNASLST |
| | YHDLLKIIKDKEFMDDPKNEEILENIVHTLTIFEDREMIKQRLAQYDSLFDEKVIKALTRRHYTGWG |
| | KLSAKLINGICDKQTNKTILDFLIDDDKINRNFMQLINDDGLSFKDIIQKAQVVGKIDDVKQVVQEL |
| | PGSPAIKKGILQSIKIVDELVKVMGHAPESIVIEMARENQTTARGKKNSQQRYKRIEDALKNLAPGL |
| | DSNILKENPTDNIQLQNDRLFLYYLQNGKDMYTGEALDINQLSNYDIDHIVPQAFIKDDSLDNRVLT |
| | SSKDNRGKSDNVPSIEVVQKRKAFWQQLLDSKLISERKENNLTKAERGGLDERDKVGFIKRQLVET |
| | RQITKHVAQILDARFNTEVNEKNQKIRKVKIITLKSNLVSNFRKEFGLYKVREINDYHHAHDAYLN |
| | AVVAKAILKKYPKLEPEFVYGDYQKYDLKRYISRSKDPKEIEKATEKYFFYSNLLNFFKEEVHYAD |
| | GTIIKRENIEYSKDTGEIAWNKEKDFATVRKVLSCPQVNIVKKTEVQTGGFSKESILPKRNSDKLIAR |
| | KKDWDPKKYGGFDSPTVAYSVLVVAKVEKGKSKKLKSVKELVGITIMERSSFEKDPVDFLEAKGY |
| | KEVRKDLIIKLPKYSLFELENGRKRMLASAGELQKGNELALPSKYVNFLYLASHYEKLKGSPEDNE |
| | QKQLFVEQHKHYLDEIIEQISEFSKRVILADANLDKVLSAYNKHRDKPIREQAENIIHLFTLTNLGAP |
| | AAFKYFDTTIDRKRYTSTKEVLDATLIHQSITGLYETRIDLSQLGGD |
| 202 | MNKPYSIGLDIGTNSVGWSVVDDDYKVPAKKMRVFGDTDKGHIKKNLLGTLLFDSGETAESRRLK |
| | RTARRRYTRRRNRILYLQEIFAKEMAKVDESFFHRLDESFLTDDDKTFDSHPIFGNKAEEDAYHQQ |
| | FPTIYHLRKHLADSTEKADLRLVYLALAHMIKFRGHFLIEGELNAENTDVQKLFADFVGVYDRTFD |
| | DSHLSEITVDASSILTEKISKSRRLEKLIKHYPTEKKNTLFGNLVALALGLQPNFKTNFKLSEDAKLQ |
| | FSKDTYEEDLEELLGKIGDDYADLFTSAKNLYDAILLSGILTVDDESTKAPLSASMIKRYAEHHEDL |
| | EKLKEFVKANKPELYHDIFKDESKNGYAGYIENGVKQDEFYKYLKETLSKQDGSQYFLDKIEREDF |
| | LRKQRTFDNGSIPHQLHLKEMRSILHRQGEYYPFLKENQAKIEKILTFRIPYYVGPLARKDSRFAWA |
| | EYHSDEPITPWNFDEVVDKEKSAEKFITRMTLNDLYLPEEKVLPKHSHVYETYTVYNELTKVKYV |
| | NEQGEAKFFDANMKQEIFDHVFKENRKVTKDKLMDFLGKEFDEFRIVDITGLDKENKAFNASLGT |
| | YHDLKKILDKSFLDDKENETIIEDIIQTLTLFEDRDMIRQRLQKYSDIFTPQQLKKLERRHYTGWGRL |
| | SYKLINGIRDKQTGKTILDYLIDDGYANRNFMQLINDDTLPFKEIIQKAQIIGDIDDIKAVVHDLPGSP |
| | AIKKGILQSVKIVDELVKVMGHNPDNIVIEMARENQTTNRGRTQSQQRLKKLQESLKELGSNILNE |
| | KKPSYVEDKVENTHLQNDRLFLYYIQNGKDMYTGDELDIDHLSDYDIDHIIPQAFIKDDSIDNRVLT |
| | SSAKNRGKSDDVPSIEIVRDRKADWMRLRKAGLISKRKFDNLTKAERGGLTEADKAGFIKRQLVET |
| | RQITKHVAQILDSRENTERDENDKVIREVKVITLKSNLVSQFRKDFQFYKVREINDYHHAHDAYLN |
| | AVVGTALIKKYPKLESEFVYGDYKVYDVRKMIAKSEQEIGKATAKRFFYSNIMNFFKTEIKLANGE |
| | IRKRPLIETNEETGEIVWNKEKDFATVRKVLSCPQVNIVKKTEVQTGGFSKESILPKRNSDKLIARKK |
| | DWDPKKYGGFDSPTVAYSVLVVAKVEKGKSKKLKSVKELLGITIMERSSFEKNPIDFLEAKGYKEV |
| | RKDLIIKLPKYSLFELENGRKRMLASAGELQKGNELALPSKYVNFLYLASHYEKLKGSPEDNEQKQ |
| | LFVEQHQHYLDEIIEQISEFSKRVILADANLDKVLSAYNKHRDKPIREQAENIIHLFTLTNLGAPAAF |
| | KYFDTTIDRKRYTSTKEVLDATLIHQSITGLYETRIDLSQLGGD |
| 203 | MKKPYSIGLDIGTNSVGWAVVTDDYKVPAKKMKVLGDTDKSHIKKNLLGTLLFESGETAEGRRLK |
| | RTARRRYTRRRNRILYLQEIFAEEMNKVDDSFFQRLDESFLKDDDKNFDSHPIFGNKAEEDAYHQK |
| | FPTIYHLRQYLADSPEKADLRLVYLALAHIIKYRGHELFEGDLKSENKDVQHLFNDFVEMFDKTVE |
| | GSYLSENLPNVADVLVEKVSKSRRLENVLHYFPNEKKNGLFGNFIALALGLQPNFKTNFKLSEDAK |
| | LQFSKDTYDEDLEELLGKIGDDYADLFVAAKNLYDAILLSGILTVTDPTTKAPLSASMIDRYEKHH |
| | KDLALLKNLIKQNLTQEKYDEVFSDESKNGYAGYISGKVKQEEFYKYLKNTLSKIEGSDYFLDKIE |
| | REDFLRKQRTFDNGSIPHQIHLQEMNAIIRRQGEHYPFLKENKEKIEKILTFRIPYYVGPLARGNRDF |
| | AWLTRNSDQAIRPWNFEEIVDQASSAEDFINKMTNYDLYLPEEKVLPKHSLLYETFAVYNELTKVK |
| | FIAEGLRDYQFLDSGQKKQIVNQLFKEKRKVTEKDIIQYLHNVDGYDGIELKGIEKQFNASLSTYHD |
| | LLKIIKDKEFMDDPKNEEILENIVHTLTIFEDREMIKQRLAQYDSLFDEKVIKALTRRHYTGWGKLS |
| | AKLINGIYDKQSKKTILDYLIDDGEINRNFMQLINDDGLSFKDIIQKAQVVGKTNDVKQVVQELPGS |
| | PAIKKGILQSIKLVDELVKVMGHAPESIVIEMARENQTTARGKKNSQQRYKRIEDALKNLAPGLDS |
| | NILKENPTDNIQLQNDRLFLYYLQNGKDMYTGEALDINQLSNYDIDHIVPQAFIKDDSLDNRVLTSS |
| | KDNRGKSDNVPSIEVVQKRKAFWQQLLDSKLISERKENNLTKAERGGLDERDKVGFIKRQLVETR |
| | QITKHVAQILDARFNTEVDENNQRIRKVKIITLKSNLVSNFRKEFGLYKVREINDYHHAHDAYLNA |
| | VVAKAILKKYPKLEPEFVYGDYQKYDLKRYISRSKDPKDIEKATEKYFFYSNLLNFFKEEVHYADG |
| | TIIKRENIEYSNDTGEIAWNKEKDFATVRKVLSCPQVNIVKKTEVQTGGFSKESILPKRNSDKLIPRK |
| | TEWDPKKYGGFDSPIVAYSVLVVAKVEKGKSKKLKPVKELVGITIMERSSFEKDPVDFLEAKGYKE |
| | VRKDLIIKLPKYSLFELENGRKRMLASAGELQKGNELALPSKYVNFLYLASHYEKLKGSPEDNEQK |
| | QLFVEQHKHYLDEIVEQISEFSERVILADANLDKILSAYNKHRDKPIREQAENIINLFTFTNLGAPAA |
| | FKFFDTDIDRKRYTSTKEVLDATLIHQSITGLYETRIDLSQLGGD |
| 204 | MKKPYSIGLDIGTNSVGWAVVTDDYKVPSKKMKVLGNTDKSHIKKNLLGALLFESGETAEGRRLK |
| | RTARRRYTRRRNRILYLQEIFAEEMNKIDESFFHRLDDSFLVPEDKRGSKYPIFATLQEEKEYHKQFP |
| | TIYHLRKQLAESNEKADLRLVYLALAHMIKYRGHFLIDDPKFKVQNNDIQGLFEKFVEEYDNVQET |
| | SLSKIKLNVTEILTANIPKSEKQEQLLKNYPSEKKNTLFGNLIGLALGLTPNFKTNFSLENDAKLQISS |
| | ESYEEDLGNLLALIGENFIELFSAVKNLSDGILLAGIVSDESPHAPLSTKMVIRFKEHEEDLAALKHFI |
| | KANLPEKYDEVFSDDSKNGYAGYVGVDSKVRKRNGKLATEEEFYKYLKDILNNVKGADYFLEKI |
| | KREDFLRKQRTFDNGTIPYQVHLTEMRAIIHRQGEHYPFLQENKEKIEKILTFRIPYYVGPLARGNR |
| | DFAWLTRNSDQAIRPWNFEEVVDKASSAEDFINKMTNYDLYLPEEKVLPKHSLLYETFAVYNELT |
| | KVKFIAEGLRDYQFLDSGQKKQIVNQLFKEKRKVTEKDIIHYLHNVDGYDGIELKGIEKQFNASLST |
| | YHDLLKIIKDKEFMDDAKNEEILENIVHTLTIFEDREMIKQRLAQYDSLFDEKVIKALTRRHYTGWG |
| | KLSAKLINGIYDKQSKKTILDYLIDDDKINRNFMQLINDDGLSFKDIIQKAQVVGKTNDVKQVVQE |
| | LPGSPAIKKGILQSIKLVDELVKVMGHAPESIVIEMARENQTTARGKKNSQQRYKRIEDALKNLAPG |
| | LDSNILKENPTDNIQLQNDRLFLYYLQNGKDMYTGEPLDINQLSSYDIDHIVPQAFIKDDSLDNRVL |
| | TSSKDNRGKSDNVPSLEVVQKRKAFWQQLLDSKLISEHKFNNLTKAERGGLDERDKVGFIKRQLV |
| | ETRQITKHVAQILDARFNTEVNEKKQKIRSVKIITLKSNLVSNFRKEFRLYKVREINDYHHAHDAYL |
| | NAVVAKAILKKYPKLEPEFVYGDYQKYDIKRYISRSKDPKEVEKATQKYFFYSNLLNFFKEEVHYA |
| | DGTIIKRENIEYSKDTGEIAWNKEKDFATVRKVLSCPQVNIVKKTEVQTGGFSKESILPKRNSDKLIP |
| | RKTEWDPKKYGGFDSPTVAYSVLVVAKVEKGKSKKLKSVKELVGITIMERSSFEKDPVDFLEAKG |
| | YKEVRKDLIIKLPKYSLFELENGRRRLLASAGELQKGNELALPSKYVNFLYLASHYEKLKGSPEDN |
| | EQKQLFVEQHKHYLDEIVEQISEFSERVILADANLDKVLSAYNKNRDRPIREQAENIIHLFTLTNLGA |
| | PAAFKFFDTTIDRKRYTSTKEVLDATLIHQSITGLYETRIDLSQLGGD |
| 205 | MNKPYSIGLDIGTNSVGWSVVDDDYKVPAKKMRVFGDTDKGHIKKNLLGTLLFDSGETAESRRLK |
| | RTARRRYTRRRNRILYLQEIFAKEMAKVDESFFHRLDESFLTDDDKTFDSHPIFGNKAEEDAYHQQ |
| | FPTIYHLRKHLADSTEKADLRLVYLALAHMIKFRGHFLIEGELNAENTDVQKLFADFVGVYDRTFD |
| | DSHLSEITVDASSILTEKISKSRRLEKLIKHYPTEKKNTLFGNLVALALGLQPNFKTNFKLSEDAKLQ |
| | FSKDTYEEDLEELLGKIGDDYADLFTSAKNLYDAILLSGILTVDDESTKAPLSASMIKRYAEHHEDL |
| | EKLKEFVKANKPELYHDIFKDESKNGYAGYIENGVKQDEFYKYLKETLSKQDGSQYFLDKIEREDF |
| | LRKQRTFDNGSIPHQLHLKEMRSILHRQGEYYPFLKENQAKIEKILTFRIPYYVGPLARKDSRFAWA |
| | EYHSDEPITPWNFDEVVDKEKSAEKFITRMTLNDLYLPEEKVLPKHSHVYETYTVYNELTKVKYV |
| | NEQGEAKFFDANMKQEIFDHVFKENRKVTKDKLMDFLGKEFDEFRIVDITGLDKENKAFNASLGT |
| | YHDLKKILDKSFLDDKENETIIEDIIQTLTLFEDRDMIRQRLQKYSDIFTPQQLKKLERRHYTGWGRL |
| | SYKLINGIRDKQTGKTILDYLIDDGYANRNFMQLINDDTLPFKEIIQKAQIIGDIDDIKAVVHDLPGSP |
| | AIKKGILQSVKIVDELVKVMGHNPDNIVIEMARENQTTNRGRTQSQQRLKKLQESLKELGSNILNE |
| | KKPSYVEDKVENTHLQNDRLFLYYIQNGKDMYTGDELDIDHLSDYDIDHIIPQAFIKDDSIDNRVLT |
| | SSAKNRGKSDDVPSIEIVRDRKADWMRLRKAGLISKRKFDNLTKAERGGLTEADKAGFIKRQLVET |
| | RQITKHVAQILDSRENTERDENDKVIREVKVITLKSNLVSQFRKDFQFYKVREINDYHHAHDAYLN |
| | AVVGTALIKKYPKLESEFVYGDYKVYDVRKMIAKSEQEIGKATAKRFFYSNIMNFFKTEIKLANGE |
| | IRKRPLIETNEETGEIVWNKEKDFATVRKVLSCPQVNIVKKTEVQTGGFSKESILPKRNSDKLIPRKT |
| | EWDPKKYGGFDSPIVAYSVLVVAKVEKGKSKKLKPVKELVGITIMERSSFEKDPVDFLEAKGYKE |
| | VRKDLIIKLPKYSLFELENGRKRMLASAGELQKGNELALPSKYVNFLYLASHYEKLKGSPEDNEQK |
| | QLFVEQHKHYLDEIVEQISEFSERVILADANLDKILSAYNKHRDKPIREQAENIINLETFTNLGAPAA |
| | FKFFDTDIDRKRYTSTKEVLDATLIHQSITGLYETRIDLSQLGGD |
| 206 | MKKPYSIGLDIGTNSVGWAVVTDDYKVPAKKMKVLGDTDKSHIKKNLLGTLLFESGETAEGRRLK |
| | RTARRRYTRRRNRILYLQEIFAEEMNKVDDSFFQRLDESFLKDDDKNFDSHPIFGNKAEEDAYHQK |
| | FPTIYHLRQYLADSPEKADLRLVYLALAHIIKYRGHELFEGDLKSENKDVQHLENDFVEMFDKTVE |
| | GSYLSENLPNVADVLVEKVSKSRRLENVLHYFPNEKKNGLFGNFIALALGLQPNFKTNFKLSEDAK |
| | LQFSKDTYDEDLEELLGKIGDDYADLFVAAKNLYDAILLSGILTVTDPTTKAPLSASMIDRYEKHH |
| | KDLALLKNLIKQNLTQEKYDEVFSDESKNGYAGYISGKVKQEEFYKYLKNTLSKIEGSDYFLDKIE |
| | REDFLRKQRTFDNGSIPHQIHLQEMNAIIRRQGEHYPFLKENKEKIEKILTFRIPYYVGPLARGNRDF |
| | AWLTRNSDQAIRPWNFEEIVDQASSAEDFINKMTNYDLYLPEEKVLPKHSLLYETFAVYNELTKVK |
| | FIAEGLRDYQFLDSGQKKQIVNQLFKEKRKVTEKDIIQYLHNVDGYDGIELKGIEKQFNASLSTYHD |
| | LLKIIKDKEFMDDPKNEEILENIVHTLTIFEDREMIKQRLAQYDSLFDEKVIKALTRRHYTGWGKLS |
| | AKLINGIYDKQSKKTILDYLIDDGEINRNFMQLINDDGLSFKDIIQKAQVVGKTNDVKQVVQELPGS |
| | PAIKKGILQSIKLVDELVKVMGHAPESIVIEMARENQTTARGKKNSQQRYKRIEDALKNLAPGLDS |
| | NILKENPTDNIQLQNDRLFLYYLQNGKDMYTGEALDINQLSNYDIDHIVPQAFIKDDSLDNRVLTSS |
| | KDNRGKSDNVPSIEVVQKRKAFWQQLLDSKLISERKENNLTKAERGGLDERDKVGFIKRQLVETR |
| | QITKHVAQILDARFNTEVDENNQRIRKVKIITLKSNLVSNFRKEFGLYKVREINDYHHAHDAYLNA |
| | VVAKAILKKYPKLEPEFVYGDYQKYDLKRYISRSKDPKDIEKATEKYFFYSNLLNFFKEEVHYADG |
| | TIIKRENIEYSNDTGEIAWNKEKDFATVRKVLSCPQVNIVKKTEVQTGGFSKESILPKRNSDKLIARK |
| | KDWDPKKYGGFDSPTVAYSVLVVAKVEKGKSKKLKSVKELVGITIMERSSFEKDPVDFLEAKGYK |
| | EVRKDLIIKLPKYSLFELENGRKRMLASAGELQKGNELALPSKYVNFLYLASHYEKLKGSPEDNEQ |
| | KQLFVEQHKHYLDEIIEQISEFSKRVILADANLDKVLSAYNKHRDKPIREQAENIIHLFTLTNLGAPA |
| | AFKYFDTTIDRKRYTSTKEVLDATLIHQSITGLYETRIDLSQLGGD |
| 207 | MKKPYSIGLDIGTNSVGWAVVTDDYKVPAKKMRVLGDTDRSHIKKNLIGALLFDAGNTAEDRRL |
| | KRTARRRYTRRRNRILYLQEIFAEEMNKVDESFFHRLDDSFLVPEDKRGSKYPIFATLQEEKEYHKQ |
| | FPTIYHLRKQLAESNEKADLRLIYLALAHMIKYRGHFLIDDPKFKVQNNDIQGLFEKFVEEYDNVQ |
| | ETSLSKIKLNVTEILTANIPKSEKQEQLLKNYPSEKKNTLFGNLIGLALGLTPNFKTNFSLENDAKLQI |
| | SSESYEEDLGNLLALIGENFIELFSAVKNLSDGILLAGIVSDESPHAPLSTKMVIRFKEHEEDLAALK |
| | HFIKANLPEKYDEVFSDDSKNGYAGYVGVDSKVRKRNGKLATEEEFYKYLKDILNNVKGADYFLE |
| | KIKREDFLRKQRTFDNGTIPYQVHLTEMRAILHRQGEYYPFLQENKEKIEKILTFRIPYYVGPLARG |
| | NRDFAWLTRNSDQAIRPWNFEEIVDQASSAEDFINKMTNYDLYLPEEKVLPKHSLLYETFAVYNEL |
| | TKVKFIAEGLRDYQFLDSGQKKQIVNQLFKEKRKVTEKDIIQYLHNVDGYDGIELKGIEKQFNASLS |
| | TYHDLLKIIKDKEFMDDPKNEEILENIVHTLTIFEDREMIKQRLAQYDSLFDEKVVKALARRHYTG |
| | WGKLSAKLINGIYDKQSKKTILDYLIDDGKINRNFMQLINDDGLSFKEIIQKAQVVGKTDDVKQVV |
| | QELPGSPAIKKGILQSIKIVDELVKVMGHAPESIVIEMARENQTTARGKKNSQQRYKRIEDALKNLA |
| | PGLDSNILKEHPTDNIQLQNDRLFLYYLQNGKDMYTGEPLDINQLSSYDIDHIVPQAFIKDDSLDNR |
| | VLTSSKDNRGKSDNVPSLEVVQKRKAFWQQLLDSKLISERKENNLTKAERGGLDERDKVGFIRRQ |
| | LVETRQITKHVAQILDARFNTEVNEKNQKIRTVKIITLKSNLVSNFRKEFGLYKVREINDYHHAHDA |
| | YLNAVLAKAILKKYPKLEPEFVYGDYQKYDLKRYISRSRDPKEIEKATEKYFFYSNLLNFFKEEVH |
| | YADGTIIKRENIEYSKDTGEIAWNKEKDFATVRKVLSCPQVNIVKKTEVQTGGFSKESILPKRNSDK |
| | LIARKKDWDPKKYGGFDSPTVAYSVLVVAKVEKGKSKKLKSVKELLGITIMERSSFEKNPIDFLEA |
| | KGYKEVRKDLIIKLPKYSLFELENGRKRMLASAGELQKGNELALPSKYVNFLYLASHYEKLKGSPE |
| | DNEQKQLFVEQHKHYLDEIIEQISEFSKRVILADANLDKVLSAYNKHRDKPIREQAENIIHLFTLTNL |
| | GAPAAFKYFDTTIDRKRYTSTKEVLDATLIHQSITGLYETRIDLSQLGGD |
| 208 | MKKSYSIGLDIGTNSVGWAVVNDDYKVPAKKMRVFGDTDRSHIKKNLLGTLLFDSGETAEGRRL |
| | KRTARRRYTRRRNRILYLQEIFAEEMNKIDESFFHRLDDSFLVPEDKRGSKYPIFATLAEEKEYHKQ |
| | FPTIYHLRKKLAESNEKADLRLVYLALAHMIKYRGHFLIDDPKFKVQNNDIQGLFEKFVEEYDNVQ |
| | ETSLSKIKLNVTEILTANIPKSEKQEQLLKNYPSEKKNTLFGNLIGLALGLTPNFKTNFSLENDAKLQI |
| | SSESYEEDLGNLLALIGENFIELFSAVKNLSDGILLAGIVSDESPHAPLSTKMVIRFKEHEEDLAALK |
| | HFIKANLPEKYDEVFSDDSKNGYAGYVGVDSKVRKRNGKLATEEEFYKYLKNILNNIKGADYFLE |
| | KIEREDLLRKQRTFDNGTIPYQVHLAEMRAILHRQGDYYPFLKENREKIEKILTFRIPYYVGPLARG |
| | NRDFAWLTRNSDQAIRPWNFEEVVDQASSAEDFINKMTNYDLYLPEEKVLPKHSLLYETFAVYNE |
| | LTKVKFIAEGLRDYQFLDSGQKKQIVNQLFKEKRKVTEKDIIHYLHNVDGYDGIELKGIEKQFNASL |
| | STYHDLLKIIKDKEFMDDPKNEEILENIVHTLTIFEDREMIKQRLAQYDSLFDEKVIKALTRRHYTG |
| | WGKLSAKLINGICDKQTGNTILDYLIDDDKINRNFMQLINDDGLSFKEIIQKAQVVGKTDDVKQVV |
| | QELPGSPAIKKGILQSIKIVDELVKVMGHAPESIVIEMARENQTTARGKKNSQQRYKRIEDALKNLA |
| | PGLDSNILKEHPTDNIQLQNDRLFLYYLQNGKDMYTGESLDINQLSSYDIDHIVPQAFIKDDSLDNR |
| | VLTSSKDNRGKSDNVPSIEVVQKRKAFWQQLLDSKLISERKENNLTKAERGGLDERDKVGFIKRQL |
| | VETRQITKHVAQILDARFNTEVNEKGQKIRKVKIITLKSNLVSNFRKEFGLYKVREINDYHHAHDA |
| | YLNAVVAKAILKKYPKLEPEFVYGDYQKYDIKRYISKSKDPKEIEKATEKYFFYSNLLNFFKEEVH |
| | YADGTIIKRENIEYSKDTGEIAWNKEKDFATVRKVLSCPQVNIVKKTEVQTGGFSKESILPKRNSDK |
| | LIPRKTEWDPKKYGGFDSPTVAYSVLVVAKVEKGKSKKLKSVKELVGITIMERSSFEKDPVDFLEA |
| | KGYKEVRKDLIIKLPKYSLFELENGRRRLLASAGELQKGNELALPSKYVNFLYLASHYEKLKGSPE |
| | DNEQKQLFVEQHKHYLDEIVEQISEFSERVILADANLDKVLSAYNKNRDRPIREQAENIIHLFTLTNL |
| | GAPAAFKFFDTTIDRKRYTSTKEVLDATLIHQSITGLYETRIDLSQLGGD |
| 209 | MKKPYSIGLDIGTNSVGWAVVTDDYKVPAKKMKVLGDTDKSHIKKNLLGTLLFESGETAEGRRLK |
| | RTARRRYTRRRNRILYLQEIFAEEMNKVDDSFFQRLDESFLKDDDKNFDSHPIFGNKAEEDAYHQK |
| | FPTIYHLRQYLADSPEKADLRLVYLALAHIIKYRGHELFEGDLKSENKDVQHLFNDFVEMEDKTVE |
| | GSYLSENLPNVADVLVEKVSKSRRLENVLHYFPNEKKNGLFGNFIALALGLQPNFKTNFKLSEDAK |
| | LQFSKDTYDEDLEELLGKIGDDYADLFVAAKNLYDAILLSGILTVTDPTTKAPLSASMIDRYEKHQ |
| | KDLALLKNLIKKNLTQEKYDEVFSDESKNGYAGYINGKVKQEEFYKYLKNTLSKIEGSDYFLDKIE |
| | REDFLRKQRTFDNGSIPHQIHLQEMNAIIRRQGEHYPFLKENKEKIEKILTFRIPYYVGPLARGNRDF |
| | AWLTRNSDQAIRPWNFEEIVDQASSAEDFINKMTNYDLYLPEEKVLPKHSLLYETFAVYNELTKVK |
| | FIAEGLRDYQFLDSGQKQQIVNQLFKEKRKVTEKDIIQYLHNVDGYDGIELKGIEKQFNASLSTYHD |
| | LLKIIKDKEFMDDPKNEEILENIVHTLTIFEDREMIKQRLAQYDSLFDEKVIKALTRRHYTGWGKLS |
| | AKLINGIYDKQSKKTILDYLIDDGEINRNFMQLINDDGLSFKDIIQKAQVVGKTNDVKQVVQELPGS |
| | PAIKKGILQSIKLVDELVKIMGHAPESIVIEMARENQTTARGKKNSQQRYKRIEDALKNLAPGLDSN |
| | ILKENPTDNIQLQNDRLFLYYLQNGKDMYTGEALDINQLSNYDIDHIVPQAFIKDDSLDNRVLTSSK |
| | DNRGKSDNVPSIEVVQKRKAFWQQLLDSKLISERKFNNLTKAERGGLDERDKVGFIKRQLVETRQI |
| | TKHVAQILDARFNTEVDENNQRIRKVKIITLKSNLVSNFRKEFGLYKVREINDYHHAHDAYLNAVV |
| | AKAILKKYPKLEPEFVYGDYQKYDLKRYISRSKDPKDIEKATEKYFFYSNLLNFFKEEVHYADGTII |
| | KRENIEYSNDTGEIAWNKEKDFAIVRKVLSCPQVNIVKKTEVQTGGFSKESILPKRNSDKLIPRKKD |
| | WDPKKYGGFDSPTVAYSVLVVAKVEKGKSKKLKSVKELVGITIMERSSFEKDPVDFLEAKGYKEV |
| | RKDLIIKLPKYSLFELENGRKRMLASAGELQKGNELALPSKYVNFLYLASHYEKLKGSPEDNEQKQ |
| | LFVEQHKHYLDEIIEQISEFSERVILADANLDKVLSAYNNHRDKPIREQAENIIHLFTLTNLGAPAAF |
| | KFFDTTIDRKRYTSTKEILNATLIHQSITGLYETRIDLSQLGGD |
| 210 | MNKPYSIGLDIGTNSVGWSVVDDDYKVPAKKMRVFGDTDKGHIKKNLLGTLLFDSGETAESRRLK |
| | RTARRRYTRRRNRILYLQEIFAKEMAKVDESFFHRLDESFLTDDDKTFDSHPIFGNKAEEDAYHQQ |
| | FPTIYHLRKHLADSTEKADLRLVYLALAHMIKFRGHFLIEGELNAENTDVQKLFADFVGVYDRTFD |
| | DSHLSEITVDASSILTEKISKSRRLEKLIKHYPTEKKNTLFGNLVALALGLQPNFKTNFKLSEDAKLQ |
| | FSKDTYEEDLEELLGKIGDDYADLFTSAKNLYDAILLSGILTVDDESTKAPLSASMIKRYAEHHEDL |
| | EKLKEFVKANKPELYHDIFKDESKNGYAGYIENGVKQDEFYKYLKETLSKQDGSQYFLDKIEREDF |
| | LRKQRTFDNGSIPHQLHLKEMRSILHRQGEYYPFLKENQAKIEKILTFRIPYYVGPLARKDSRFAWA |
| | EYHSDEPITPWNFDEVVDKEKSAEKFITRMTLNDLYLPEEKVLPKHSHVYETYTVYNELTKVKYV |
| | NEQGEAKFFDANMKQEIFDHVFKENRKVTKDKLMDFLGKEFDEFRIVDITGLDKENKAFNASLGT |
| | YHDLKKILDKSFLDDKENETIIEDIIQTLTLFEDRDMIRQRLQKYSDIFTPQQLKKLERRHYTGWGRL |
| | SYKLINGIRDKQTGKTILDYLIDDGYANRNFMQLINDDTLPFKEIIQKAQIIGDIDDIKAVVHDLPGSP |
| | AIKKGILQSVKIVDELVKVMGHNPDNIVIEMARENQTTNRGRTQSQQRLKKLQESLKELGSNILNE |
| | KKPSYVEDKVENTHLQNDRLFLYYIQNGKDMYTGDELDIDHLSDYDIDHIIPQAFIKDDSIDNRVLT |
| | SSAKNRGKSDDVPSIEIVRDRKADWMRLRKAGLISKRKFDNLTKAERGGLTEADKAGFIKRQLVET |
| | RQITKHVAQILDSRENTERDENDKVIREVKVITLKSNLVSQFRKDFQFYKVREINDYHHAHDAYLN |
| | AVVGTALIKKYPKLESEFVYGDYKVYDVRKMIAKSEQEIGKATAKRFFYSNIMNFFKTEIKLANGE |
| | IRKRPLIETNEETGEIVWNKEKDFATVRKVLSCPQVNIVKKTEVQTGGFSKESILPKRNSDKLIARKK |
| | DWDPKKYGGFDSPTVAYSVLVVAKVEKGKSKKLKSVKELLGITIMERSSFEKNPIDFLEAKGYKEV |
| | RKDLIIKLPKYSLFELENGRKRMLASAGELQKGNELALPSKYVNFLYLASHYEKLKGSPEDNEQKQ |
| | LFVEQHKHYLDEIIEQISEFSKRVILADANLDKVLSAYNKHRDKPIREQAENIIHLFTLTNLGAPAAF |
| | KYFDTTIDRKRYTSTKEVLDATLIHQSITGLYETRIDLSQLGGD |
| 211 | MKKPYSIGLDIGTNSVGWAVVTDDYKVPAKKMKVLGDTDRSHIKKNLLGALLFDSGETAEGRRL |
| | KRTARRRYTRRRNRILYLQEIFAEEMNKVDESFFHRLDDSFLVPEDKRGSKYPIFATLEEEKEYHKQ |
| | FPTIYHLRKKLAESNEKADLRLIYLALAHMIKYRGHFLIDDPKFKVQNNDIQGLFEKFVEEYDNVQ |
| | ETSLSKIKLNVTEILTAKIPKSEKQEQLLKNYPSEKKNTLFGNLIGLALGLTPNFKTNFSLENDAKLQI |
| | SSESYEEDLGNLLALIGENFIELFSAVKNLSDGILLAGIVSDESPHAPLSTKMVIRFKEHEEDLAALK |
| | HFIKSNLPEKYDEVFSDDSKNGYAGYVGVDSKVRKRNGKLATEEEFYKYLKDILNNVKGADYFLE |
| | KIKREDFLRKQRTFDNGVIPYQVHLTEMKAILQHQGEYYPFLQENKEKIQQILTFRIPYYVGPLARG |
| | NRDFAWLTRNSDQAIRPWNFEEVVDQASSAEDFINKMTNYDLYLPEEKVLPKHSLLYETFAVYNE |
| | LTKVKFIAEGLRDYQFLDSGQKKQIVNQLFKEKRKVTEKDIIHYLHNVDGYDGIELKGIEKQFNASL |
| | STYHDLLKIIKDKEFMDDPKNEEILENIVHTLTIFEDREMIKQRLAQYDSLFDEKVIKALTRRHYTG |
| | WGKLSAKLINGICDKQTGDTILDYLIDDDKINRNFMQLINDDGLSFKEIIQKAQVVGKTDDVKQVV |
| | QELPGSPAIKKGILQSIKLVDELVKVMGHAPESIVIEMARENQTTARGKKNSQQRYKRIEDALKNL |
| | APGLDSNILKEHPTDNIQLQNDRLFLYYLQNGRDMYTGEPLDINQLSNYDIDHIVPQAFIKDDSLDN |
| | RVLTSSKDNRGKSDNVPSLEVVQKRKAFWQQLLDSKLISERKENNLTKAERGGLDERDKVGFIKR |
| | QLVETRQITKHVAQILDARFNTEVNEKNQKIRKVKIITLKSNLVSNFRKEFGLYKVREINDYHHAHD |
| | AYLNAVVAKAILKKYPKLEPEFVYGDYQKYDLKRYISRSKDPKEIEKATEKYFFYSNLLNFFKEEV |
| | HYADGTIIKRENIEYSKDTGEIAWNKEKDFATVRKVLSCPQVNIVKKTEVQTGGFSKESILPKRNSD |
| | KLIARKKDWDPKKYGGFDSPTVAYSVLVVAKVEKGKSKKLKSVKELLGITIMERSSFEKNPIDFLE |
| | AKGYKEVRKDLIIKLPKYSLFELENGRKRMLASAGELQKGNELALPSKYVNFLYLASHYEKLKGSP |
| | EDNEQKQLFVEQHKHYLDEIIEQISEFSKRVILADANLDKVLSAYNKHRDKPIREQAENIIHLFTLTN |
| | LGAPAAFKYFDTTIDRKRYTSTKEVLDATLIHQSITGLYETRIDLSQLGGD |
| 212 | MKKSYSIGLDIGTNSVGWSVITDDYKVPAKKMKVLGNTDKKYIKKNLLGALLFDSGETAEATRLK |
| | RTARRRYTRRKNRLRYLQEIFAKEMAKVDESFFQRLEESFLTDDDKNFDSHPIFGNKAEEDAYHDE |
| | FPTIYHLRQYLADSPQKADLRLVYLALAHMIKFRGHFLIEGQLNAENTDVQTLFNDFVEVYDKTVE |
| | ESHLSEITVDALSILTEKVSKSRRLENLVKCYPTEKKNTLFGNLIALSLGLQPNFKTNFKLSEDAKLQ |
| | FSKDTYEEDLEELLGKIGDEYADLFASAKNLYDAILLSGILTVDDNTTKAPLSASMVKRYAEHHED |
| | LEKLKEFVKANKPELYHDIFKDETKNGYAGYIENGVKQDEFYKYLKNTLSKIAGSDYFLDKIERED |
| | FLRKQRTFDNGSIPHQIHLQEMHAILRRQGEYYPFLKENQAKIEKILTFRIPYYVGPLARKNSRFAW |
| | AEYHSDEKITPWNFEEIVDKEESATAFIHRMTNNDLYLPEEKVLPKHSLIYEEFTVYNELTKVKYVT |
| | DQGKAYYFDANMKQEIFDGVFKEHRKVTKDKLMNFLEKEFDEFRIVDLTGLDKENKSFNASLGTY |
| | HDLKKILDKSFLDDKTNETIIEDIIQTLTLFEDRDMIRQRLQKYSDIFTPQQLKKLERRHYTGWGRLS |
| | YKLINGIRNKENGKTILDYLIDDGYANRNFMQLINDDTLPFKQIIQEAQIIGDIDDIETVVHDLPGSPA |
| | IKKGILQSVKIVDELVKVMGHNPDNIVIEMARENQTTNRGRSQSQQRLKKLQESLKELGSNILNEEK |
| | PSYVEDKVENSHLQNDKLFLYYIQNGKDMYTGEELDIDHLSDYDIDHIIPQAFIKDNSIDNRVLTSS |
| | AKNRGKSDDVPSLDIVHDRKADWIRLYKSGLISKRKFDNLTKAEHGGLTEDDKAGFIKRQLVETR |
| | QITKHVAQILDSRENTERDENDKVIRDVKVITLKSNLVSQFRKDFELHKVREINDYHHAHDAYLNA |
| | VIGNALLVKYPQLEPEFVYGKFKHFHGHKENKATAKKFFYSNIMALFKKDVEYSDDTGEMVWNK |
| | EKDFATVRKVLSCPQVNIVKKTEVQTGGFSKESILPKRNSDKLIARKKDWDPKKYGGFDSPTVAYS |
| | VLVVAKVEKGKSKKLKSVKELVGITIMERSSFEKDPVDFLEAKGYKEVRKDLIIKLPKYSLFELENG |
| | RKRMLASAGELQKGNELALPSKYVNFLYLASHYEKLKGSPEDNEQKQLFVEQHKHYLDEIIEQISE |
| | FSKRVILADANLDKVLSAYNKHRDKPIREQAENIIHLFTLTNLGAPAAFKYFDTTIDRKRYTSTKEV |
| | LDATLIHQSITGLYETRIDLSQLGGD |
| 213 | MNKPYSIGLDIGTNSVGWSVVDDDYKVPAKKMRVFGDTDKGHIKKNLLGTLLFDSGETAESRRLK |
| | RTARRRYTRRRNRILYLQEIFAKEMAKVDESFFHRLDESFLTDDDKTFDSHPIFGNKAEEDAYHQQ |
| | FPTIYHLRKHLADSTEKADLRLVYLALAHMIKFRGHFLIEGELNAENTDVQKLFADFVGVYDRTFD |
| | DSHLSEITVDASSILTEKISKSRRLEKLIKHYPTEKKNTLFGNLVALALGLQPNFKTNFKLSEDAKLQ |
| | FSKDTYEEDLEELLGKIGDDYADLFTSAKNLYDAILLSGILTVDDESTKAPLSASMIKRYAEHHEDL |
| | EKLKEFVKANKPELYHDIFKDESKNGYAGYIENGVKQDEFYKYLKETLSKQDGSQYFLDKIEREDF |
| | LRKQRTFDNGSIPHQLHLKEMRSILHRQGEYYPFLKENQAKIEKILTFRIPYYVGPLARKDSRFAWA |
| | EYHSDEPITPWNFDEVVDKEKSAEKFITRMTLNDLYLPEEKVLPKHSHVYETYTVYNELTKVKYV |
| | NEQGEAKFFDANMKQEIFDHVFKENRKVTKDKLMDFLGKEFDEFRIVDITGLDKENKAFNASLGT |
| | YHDLKKILDKSFLDDKENETIIEDIIQTLTLFEDRDMIRQRLQKYSDIFTPQQLKKLERRHYTGWGRL |
| | SYKLINGIRDKQTGKTILDYLIDDGYANRNFMQLINDDTLPFKEIIQKAQIIGDIDDIKAVVHDLPGSP |
| | AIKKGILQSVKIVDELVKVMGHNPDNIVIEMARENQTTNRGRTQSQQRLKKLQESLKELGSNILNE |
| | KKPSYVEDKVENTHLQNDRLFLYYIQNGKDMYTGDELDIDHLSDYDIDHIIPQAFIKDDSIDNRVLT |
| | SSAKNRGKSDDVPSIEIVRDRKADWMRLRKAGLISKRKFDNLTKAERGGLTEADKAGFIKRQLVET |
| | RQITKHVAQILDSRENTERDENDKVIREVKVITLKSNLVSQFRKDFQFYKVREINDYHHAHDAYLN |
| | AVVGTALIKKYPKLESEFVYGDYKVYDVRKMIAKSEQEIGKATAKRFFYSNIMNFFKTEIKLANGE |
| | IRKRPLIETNEETGEIVWNKEKDFATVRKVLSCPQVNIVKKTEVQTGGFSKESILPKRNSDKLIARKK |
| | DWDPKKYGGFDSPTVAYSVLVVAKVEKGKSKKLKSVKELVGITIMERSSFEKNPIDFLEAKGYKE |
| | VRKDLIIKLPKYSLFELENGRKRMLASAGELQKGNELALPSKYVNFLYLASHYEKLKGSPEDNEQK |
| | QLFVEQHKHYLDEIIEQISEFSKRVILADANLDKVLSAYNKHRDKPIREQAENIIHLFTLTNLGAPAA |
| | FKYFDTTIDRKRYTSTKEVLDATLIHQSITGLYETRIDLSQLGGD |
| 214 | MKKPYSIGLDIGTNSVGWAVVTDDYKVPAKKMRVLGDTDRSHIKKNLIGALLFDAGNTAEDRRL |
| | KRTARRRYTRRRNRILYLQEIFAEEMNKVDESFFHRLDDSFLVPEDKRGSKYPIFATLQEEKEYHKQ |
| | FPTIYHLRKQLAESNEKADLRLIYLALAHMIKYRGHFLIDDPKFKVQNNDIQGLFEKFVEEYDNVQ |
| | ETSLSKIKLNVTEILTANIPKSEKQEQLLKNYPSEKKNTLFGNLIGLALGLTPNFKTNFSLENDAKLQI |
| | SSESYEEDLGNLLALIGENFIELFSAVKNLSDGILLAGIVSDESPHAPLSTKMVIRFKEHEEDLAALK |
| | HFIKANLPEKYDEVFSDDSKNGYAGYVGVDSKVRKRNGKLATEEEFYKYLKDILNNVKGADYFLE |
| | KIKREDFLRKQRTFDNGTIPYQVHLTEMRAILHRQGEYYPFLQENKEKIEKILTFRIPYYVGPLARG |
| | NRDFAWLTRNSDQAIRPWNFEEIVDQASSAEDFINKMTNYDLYLPEEKVLPKHSLLYETFAVYNEL |
| | TKVKFIAEGLRDYQFLDSGQKKQIVNQLFKEKRKVTEKDIIQYLHNVDGYDGIELKGIEKQFNASLS |
| | TYHDLLKIIKDKEFMDDPKNEEILENIVHTLTIFEDREMIKQRLAQYDSLFDEKVVKALARRHYTG |
| | WGKLSAKLINGIYDKQSKKTILDYLIDDGKINRNFMQLINDDGLSFKEIIQKAQVVGKTDDVKQVV |
| | QELPGSPAIKKGILQSIKIVDELVKVMGHAPESIVIEMARENQTTARGKKNSQQRYKRIEDALKNLA |
| | PGLDSNILKEHPTDNIQLQNDRLFLYYLQNGKDMYTGEPLDINQLSSYDIDHIVPQAFIKDDSLDNR |
| | VLTSSKDNRGKSDNVPSLEVVQKRKAFWQQLLDSKLISERKENNLTKAERGGLDERDKVGFIRRQ |
| | LVETRQITKHVAQILDARFNTEVNEKNQKIRTVKIITLKSNLVSNFRKEFGLYKVREINDYHHAHDA |
| | YLNAVLAKAILKKYPKLEPEFVYGDYQKYDLKRYISRSRDPKEIEKATEKYFFYSNLLNFFKEEVH |
| | YADGTIIKRENIEYSKDTGEIAWNKEKDFATVRKVLSCPQVNIVKKTEVQTGGFSKESILPKRNSDK |
| | LIPRKTEWDPKKYGGFDSPTVAYSVLVVAKVEKGKSKKLKSVKELVGITIMERSSFEKDPVDFLEA |
| | KGYKEVRKDLIIKLPKYSLFELENGRRRLLASAGELQKGNELALPSKYVNFLYLASHYEKLKGSPE |
| | DNEQKQLFVEQHKHYLDEIVEQISEFSERVILADANLDKVLSAYNKNRDRPIREQAENIIHLFTLTNL |
| | GAPAAFKFFDTTIDRKRYTSTKEVLDATLIHQSITGLYETRIDLSQLGGD |
| 215 | MNKPYSIGLDIGTNSVGWSVVDDDYKVPAKKMRVFGDTDKGHIKKNLLGTLLFDSGETAESRRLK |
| | RTARRRYTRRRNRILYLQEIFAKEMAKVDESFFHRLDESFLTDDDKTFDSHPIFGNKAEEDAYHQQ |
| | FPTIYHLRKHLADSTEKADLRLVYLALAHMIKFRGHFLIEGELNAENTDVQKLFADFVGVYDRTFD |
| | DSHLSEITVDASSILTEKISKSRRLEKLIKHYPTEKKNTLFGNLVALALGLQPNFKTNFKLSEDAKLQ |
| | FSKDTYEEDLEELLGKIGDDYADLFTSAKNLYDAILLSGILTVDDESTKAPLSASMIKRYAEHHEDL |
| | EKLKEFVKANKPELYHDIFKDESKNGYAGYIENGVKQDEFYKYLKETLSKQDGSQYFLDKIEREDF |
| | LRKQRTFDNGSIPHQLHLKEMRSILHRQGEYYPFLKENQAKIEKILTFRIPYYVGPLARKDSRFAWA |
| | EYHSDEPITPWNFDEVVDKEKSAEKFITRMTLNDLYLPEEKVLPKHSHVYETYTVYNELTKVKYV |
| | NEQGEAKFFDANMKQEIFDHVFKENRKVTKDKLMDFLGKEFDEFRIVDITGLDKENKAFNASLGT |
| | YHDLKKILDKSFLDDKENETIIEDIIQTLTLFEDRDMIRQRLQKYSDIFTPQQLKKLERRHYTGWGRL |
| | SYKLINGIRDKQTGKTILDYLIDDGYANRNFMQLINDDTLPFKEIIQKAQIIGDIDDIKAVVHDLPGSP |
| | AIKKGILQSVKIVDELVKVMGHNPDNIVIEMARENQTTNRGRTQSQQRLKKLQESLKELGSNILNE |
| | KKPSYVEDKVENTHLQNDRLFLYYIQNGKDMYTGDELDIDHLSDYDIDHIIPQAFIKDDSIDNRVLT |
| | SSAKNRGKSDDVPSIEIVRDRKADWMRLRKAGLISKRKFDNLTKAERGGLTEADKAGFIKRQLVET |
| | RQITKHVAQILDSRENTERDENDKVIREVKVITLKSNLVSQFRKDFQFYKVREINDYHHAHDAYLN |
| | AVVGTALIKKYPKLESEFVYGDYKVYDVRKMIAKSEQEIGKATAKRFFYSNIMNFFKTEIKLANGE |
| | IRKRPLIETNEETGEIVWNKEKDFATVRKVLSCPQVNIVKKTEVQTGGFSKESILPKRNSDKLIPRKT |
| | EWDPKKYGGFDSPTVAYSVLVVAKVEKGKSKKLKSVKELVGITIMERSSFEKDPVDFLEAKGYKE |
| | VRKDLIIKLPKYSLFELENGRRRLLASAGELQKGNELALPSKYVNFLYLASHYEKLKGSPEDNEQK |
| | QLFVEQHKHYLDEIVEQISEFSERVILADANLDKVLSAYNKNRDRPIREQAENIIHLFTLTNLGAPAA |
| | FKFFDTTIDRKRYTSTKEVLDATLIHQSITGLYETRIDLSQLGGD |
| 216 | MKKPYSIGLDIGTNSVGWAVVTDDYKVPAKKMKVLGDTDKSHIKKNLLGTLLFESGETAEGRRLK |
| | RTARRRYTRRRNRILYLQEIFAEEMNKVDDSFFQRLDESFLKDDDKNFDSHPIFGNKAEEDAYHQK |
| | FPTIYHLRQYLADSPEKADLRLVYLALAHIIKYRGHFLFEGDLKSENKDVQHLFNDFVEMFDKTVE |
| | GSYLSENLPNVADVLVEKVSKSRRLENVLHYFPNEKKNGLFGNFIALALGLQPNFKTNFKLSEDAK |
| | LQFSKDTYDEDLEELLGKIGDDYADLFVAAKNLYDAILLSGILTVTDPTTKAPLSASMIDRYEKHH |
| | KDLALLKNLIKQNLTQEKYDEVFSDESKNGYAGYISGKVKQEEFYKYLKNTLSKIEGSDYFLDKIE |
| | REDFLRKQRTFDNGSIPHQIHLQEMNAIIRRQGEHYPFLKENKEKIEKILTFRIPYYVGPLARGNRDF |
| | AWLTRNSDQAIRPWNFEEIVDQASSAEDFINKMTNYDLYLPEEKVLPKHSLLYETFAVYNELTKVK |
| | FIAEGLRDYQFLDSGQKKQIVNQLFKEKRKVTEKDIIQYLHNVDGYDGIELKGIEKQFNASLSTYHD |
| | LLKIIKDKEFMDDPKNEEILENIVHTLTIFEDREMIKQRLAQYDSLFDEKVIKALTRRHYTGWGKLS |
| | AKLINGIYDKQSKKTILDYLIDDGEINRNFMQLINDDGLSFKDIIQKAQVVGKTNDVKQVVQELPGS |
| | PAIKKGILQSIKLVDELVKVMGHAPESIVIEMARENQTTARGKKNSQQRYKRIEDALKNLAPGLDS |
| | NILKENPTDNIQLQNDRLFLYYLQNGKDMYTGEALDINQLSNYDIDHIVPQAFIKDDSLDNRVLTSS |
| | KDNRGKSDNVPSIEVVQKRKAFWQQLLDSKLISERKENNLTKAERGGLDERDKVGFIKRQLVETR |
| | QITKHVAQILDARFNTEVDENNQRIRKVKIITLKSNLVSNFRKEFGLYKVREINDYHHAHDAYLNA |
| | VVAKAILKKYPKLEPEFVYGDYQKYDLKRYISRSKDPKDIEKATEKYFFYSNLLNFFKEEVHYADG |
| | TIIKRENIEYSNDTGEIAWNKEKDFATVRKVLSCPQVNIVKKTEVQTGGFSKESILPKRNSDKLIARK |
| | KDWDPKKYGGFDSPTVAYSVLVVAKVEKGKSKKLKSVKELVGITIMERSSFEKNPIDFLEAKGYK |
| | EVRKDLIIKLPKYSLFELENGRKRMLASAGELQKGNELALPSKYVNFLYLASHYEKLKGSPEDNEQ |
| | KQLFVEQHKHYLDEIIEQISEFSKRVILADANLDKVLSAYNKHRDKPIREQAENIIHLFTLTNLGAPA |
| | AFKYFDTTIDRKRYTSTKEVLDATLIHQSITGLYETRIDLSQLGGD |
| 217 | MKKPYSIGLDIGTNSVGWAVVTDDYKVPAKKMKVLGDTDKSHIKKNLLGTLLFESGETAEGRRLK |
| | RTARRRYTRRRNRILYLQEIFAEEMNKVDDSFFQRLDESFLKDDDKNFDSHPIFGNKAEEDAYHQK |
| | FPTIYHLRQYLADSPEKADLRLVYLALAHIIKYRGHELFEGDLKSENKDVQHLFNDFVEMEDKTVE |
| | GSYLSENLPNVADVLVEKVSKSRRLENVLHYFPNEKKNGLFGNFIALALGLQPNFKTNFKLSEDAK |
| | LQFSKDTYDEDLEELLGKIGDDYADLFVAAKNLYDAILLSGILTVTDPTTKAPLSASMIDRYEKHH |
| | KDLALLKNLIKQNLTQEKYDEVFSDESKNGYAGYISGKVKQEEFYKYLKNTLSKIEGSDYFLDKIE |
| | REDFLRKQRTFDNGSIPHQIHLQEMNAIIRRQGEHYPFLKENKEKIEKILTFRIPYYVGPLARGNRDF |
| | AWLTRNSDQAIRPWNFEEIVDQASSAEDFINKMTNYDLYLPEEKVLPKHSLLYETFAVYNELTKVK |
| | FIAEGLRDYQFLDSGQKKQIVNQLFKEKRKVTEKDIIQYLHNVDGYDGIELKGIEKQFNASLSTYHD |
| | LLKIIKDKEFMDDPKNEEILENIVHTLTIFEDREMIKQRLAQYDSLFDEKVIKALTRRHYTGWGKLS |
| | AKLINGIYDKQSKKTILDYLIDDGEINRNFMQLINDDGLSFKDIIQKAQVVGKTNDVKQVVQELPGS |
| | PAIKKGILQSIKLVDELVKVMGHAPESIVIEMARENQTTARGKKNSQQRYKRIEDALKNLAPGLDS |
| | NILKENPTDNIQLQNDRLFLYYLQNGKDMYTGEALDINQLSNYDIDHIVPQAFIKDDSLDNRVLTSS |
| | KDNRGKSDNVPSIEVVQKRKAFWQQLLDSKLISERKENNLTKAERGGLDERDKVGFIKRQLVETR |
| | QITKHVAQILDARFNTEVDENNQRIRKVKIITLKSNLVSNFRKEFGLYKVREINDYHHAHDAYLNA |
| | VVAKAILKKYPKLEPEFVYGDYQKYDLKRYISRSKDPKDIEKATEKYFFYSNLLNFFKEEVHYADG |
| | TIIKRENIEYSNDTGEIAWNKEKDFATVRKVLSCPQVNIVKKTEVQTGGFSKESILPKRNSDKLIARK |
| | KDWDPKKYGGFDSPTVAYSVLVVAKVEKGKSKKLKSVKELVGITIMERSSFEKDPVDFLEAKGYK |
| | EVRKDLIIKLPKYSLFELENGRKRMLASAGELQKGNELALPSKYVNFLYLASHYEKLKGSPEDNEQ |
| | KQLFVEQHKHYLDEIIEQISEFSKRVILADANLDKVLSAYNKHRDKPIREQAENIIHLFTLTNLGAPA |
| | AFKYFDTTIDRKRYTSTKEVLDSTLIHQSITGLYETRIDLSQLGGD |
| 218 | MNKPYSIGLDIGTNSVGWSVVDDDYKVPAKKMRVFGDTDKGHIKKNLLGTLLFDSGETAESRRLK |
| | RTARRRYTRRRNRILYLQEIFAKEMAKVDESFFHRLDESFLTDDDKTFDSHPIFGNKAEEDAYHQQ |
| | FPTIYHLRKHLADSTEKADLRLVYLALAHMIKFRGHFLIEGELNAENTDVQKLFADFVGVYDRTFD |
| | DSHLSEITVDASSILTEKISKSRRLEKLIKHYPTEKKNTLFGNLVALALGLQPNFKTNFKLSEDAKLQ |
| | FSKDTYEEDLEELLGKIGDDYADLFTSAKNLYDAILLSGILTVDDESTKAPLSASMIKRYAEHHEDL |
| | EKLKEFVKANKPELYHDIFKDESKNGYAGYIENGVKQDEFYKYLKETLSKQDGSQYFLDKIEREDF |
| | LRKQRTFDNGSIPHQLHLKEMRSILHRQGEYYPFLKENQAKIEKILTFRIPYYVGPLARKDSRFAWA |
| | EYHSDEPITPWNFDEVVDKEKSAEKFITRMTLNDLYLPEEKVLPKHSHVYETYTVYNELTKVKYV |
| | NEQGEAKFFDANMKQEIFDHVFKENRKVTKDKLMDFLGKEFDEFRIVDITGLDKENKAFNASLGT |
| | YHDLKKILDKSFLDDKENETIIEDIIQTLTLFEDRDMIRQRLQKYSDIFTPQQLKKLERRHYTGWGRL |
| | SYKLINGIRDKQTGKTILDYLIDDGYANRNFMQLINDDTLPFKEIIQKAQIIGDIDDIKAVVHDLPGSP |
| | AIKKGILQSVKIVDELVKVMGHNPDNIVIEMARENQTTNRGRTQSQQRLKKLQESLKELGSNILNE |
| | KKPSYVEDKVENTHLQNDRLFLYYIQNGKDMYTGDELDIDHLSDYDIDHIIPQAFIKDDSIDNRVLT |
| | SSAKNRGKSDDVPSIEIVRDRKADWMRLRKAGLISKRKFDNLTKAERGGLTEADKAGFIKRQLVET |
| | RQITKHVAQILDSRENTERDENDKVIREVKVITLKSNLVSQFRKDFQFYKVREINDYHHAHDAYLN |
| | AVVGTALIKKYPKLESEFVYGDYKVYDVRKMIAKSEQEIGKATAKRFFYSNIMNFFKTEIKLANGE |
| | IRKRPLIETNEETGEIVWNKEKDFATVRKVLSCPQVNIVKKTEVQTGGFSKESILPKRNSDKLIARKK |
| | DWDPKKYGGFDSPTVAYSVLVVAKVEKGKSKKLKSVKELVGITIMERSSFEKDPIDFLEAKGYKE |
| | VRKDLIIKLPKYSLFELENGRKRMLASAGELQKGNELALPSKYVNFLYLASHYEKLKGSPEDNEQK |
| | QLFVEQHKHYLDEIIEQISEFSKRVILADANLDKVLSAYNKHRDKPIREQAENIIHLFTLTNLGAPAA |
| | FKYFDTTIDRKRYTSTKEVLDATLIHQSITGLYETRIDLSQLGGD |
| 219 | MKKSYSIGLDIGTNSVGWSVITDDYKVPAKKMKVLGNTDKKYIKKNLLGALLFDSGETAEATRLK |
| | RTARRRYTRRKNRLRYLQEIFAKEMAKVDESFFQRLEESFLTDDDKNFDSHPIFGNKAEEDAYHDE |
| | FPTIYHLRQYLADSPQKADLRLVYLALAHMIKFRGHFLIEGQLSAENTNVQTLFNDFVEVYDKTVE |
| | ESHLSEITIDALSILTEKVSKSRRLENLVKCYPTEKKNTLFGNLIALSLGLQPNFKTNFKLSEDAKLQF |
| | SKDTYEEDLEELLGKIGDEYADLFASAKNLYDAILLSGILTVDDNSTKAPLSASMVKRYAEHHEDL |
| | EKLKEFVKANKPELYHDIFKDETKNGYAGYIENGVKQDEFYKYLKNTLSKIAGSDYFLDKIEREDF |
| | LRKQRTFDNGSIPHQIHLQEMHAILRRQGEYYPFLKENQAKIEKILTFRIPYYVGPLARKNSRFAWA |
| | EYHSDEKITPWNFEEIVDKEESATAFIHRMTNNDLYLPEEKVLPKHSLIYEEFTVYNELTKVKYVTD |
| | QGKAYYFDANMKQEIFDGVFKEHRKVTKDKLMNFLEKEFDEFRIVDLTGLDKENKSFNASLGTYH |
| | DLKKILDKSFLDDKTNETIIEDIIQTLTLFEDRDMIRQRLQKYSDIFTPQQLKKLERRHYTGWGRLSY |
| | KLINGIRNKENRKTILDYLIDDGYANRNFMQLINDDTLPFKQIIQEAQIIGDIDDIETVVHDLPGSPAI |
| | KKGILQSVKIVDELVKVMGHNPDNIVIEMARENQTTNRGRSQSQQRLKKLQESLKELGSNILNEEK |
| | PSYVEDKVENSHLQNDKLFLYYIQNGKDMYTGEELDIDHLSDYDIDHIIPQAFIKDNSIDNRVLTSS |
| | AKNRGKSDDVPSLDIVHDRKADWIRLYKSGLISKRKFDNLTKAEHGGLTEDDKAGFIKRQLVETR |
| | QITKHVAQILDSRENTERDENDKVIRDVKVITLKSNLVSQFRKDFELHKVREINDYHHAHDAYLNA |
| | VIGNALLVKYPQLEPEFVYGKFSHFHGHKENKATAKKFFYSNIMALFKKDVEYSDDTGEMVWNK |
| | EKDFATVRKVLSCPQVNIVKKTEVQTGGFSKESILPKRNSDKLIPRKKDWDPKKYGGFDSPTVAYS |
| | VLVVAKVEKGKSKKLKPVKELIGITIMERSSFEKDPIDFLEAKGYKEVRKDLIIKLPKYSLFELENGR |
| | KRMLASAGELQKGNELALPSKYVNFLYLASHYEKLKGSPEDNEQKQLFVEQHKHYLDEIIEQISEF |
| | SERVILADANLDKVLSAYNKHRDKPIREQAENILHLFTLTNLGAPAAFKFFDTTIDRKRYTSTKEVL |
| | DATLIHQSITGLYETRIDLSQLGGD |
| 220 | MNKPYSIGLDIGTNSVGWSVVDDDYKVPAKKMRVFGDTDKGHIKKNLLGTLLFDSGETAESRRLK |
| | RTARRRYTRRRNRILYLQEIFAKEMAKVDESFFHRLDESFLTDDDKTFDSHPIFGNKAEEDAYHQQ |
| | FPTIYHLRKHLADSTEKADLRLVYLALAHMIKFRGHFLIEGELNAENTDVQKLFADFVGVYDRTFD |
| | DSHLSEITVDASSILTEKISKSRRLEKLIKHYPTEKKNTLFGNLVALALGLQPNFKTNFKLSEDAKLQ |
| | FSKDTYEEDLEELLGKIGDDYADLFTSAKNLYDAILLSGILTVDDESTKAPLSASMIKRYAEHHEDL |
| | EKLKEFVKANKPELYHDIFKDESKNGYAGYIENGVKQDEFYKYLKETLSKQDGSQYFLDKIEREDF |
| | LRKQRTFDNGSIPHQLHLKEMRSILHRQGEYYPFLKENQAKIEKILTFRIPYYVGPLARKDSRFAWA |
| | EYHSDEPITPWNFDEVVDKEKSAEKFITRMTLNDLYLPEEKVLPKHSHVYETYTVYNELTKVKYV |
| | NEQGEAKFFDANMKQEIFDHVFKENRKVTKDKLMDFLGKEFDEFRIVDITGLDKENKAFNASLGT |
| | YHDLKKILDKSFLDDKENETIIEDIIQTLTLFEDRDMIRQRLQKYSDIFTPQQLKKLERRHYTGWGRL |
| | SYKLINGIRDKQTGKTILDYLIDDGYANRNFMQLINDDTLPFKEIIQKAQIIGDIDDIKAVVHDLPGSP |
| | AIKKGILQSVKIVDELVKVMGHNPDNIVIEMARENQTTNRGRTQSQQRLKKLQESLKELGSNILNE |
| | KKPSYVEDKVENTHLQNDRLFLYYIQNGKDMYTGDELDIDHLSDYDIDHIIPQAFIKDDSIDNRVLT |
| | SSAKNRGKSDDVPSIEIVRDRKADWMRLRKAGLISKRKFDNLTKAERGGLTEADKAGFIKRQLVET |
| | RQITKHVAQILDSRENTERDENDKVIREVKVITLKSNLVSQFRKDFQFYKVREINDYHHAHDAYLN |
| | AVVGTALIKKYPKLESEFVYGDYKVYDVRKMIAKSEQEIGKATAKRFFYSNIMNFFKTEIKLANGE |
| | IRKRPLIETNEETGEIVWNKEKDFATVRKVLSCPQVNIVKKTEVQTGGFSKESILPKRNSDKLIPRKK |
| | GWDPKKYGGFDSPTVAYSVLVVAKVEKGKSKKLKSVKELVGITIMERSSFEKDPVDFLEAKGYKE |
| | VRKDLIIKLPKYSLFELENGRKRMLASAGELQKGNELALPSKYVNFLYLASHYEKLKGSPEDNEQK |
| | QLFVEQHKHYLDEIVEQISEFSERVILADANLDKVLSAYNKNRDRPIREQAENIIHLFTLTNLGAPAA |
| | FKFFNTDIDRKRYTSTKEILDATLIHQSITGLYETRIDLSQLGGD |
| 221 | MNKPYSIGLDIGTNSVGWSVVDDDYKVPAKKMRVFGDTDKGHIKKNLLGTLLFDSGETAESRRLK |
| | RTARRRYTRRRNRILYLQEIFAKEMAKVDESFFHRLDESFLTDDDKTFDSHPIFGNKAEEDAYHQQ |
| | FPTIYHLRKHLADSTEKADLRLVYLALAHMIKFRGHFLIEGELNAENTDVQKLFADFVGVYDRTFD |
| | DSHLSEITVDASSILTEKISKSRRLEKLIKHYPTEKKNTLFGNLVALALGLQPNFKTNFKLSEDAKLQ |
| | FSKDTYEEDLEELLGKIGDDYADLFTSAKNLYDAILLSGILTVDDESTKAPLSASMIKRYAEHHEDL |
| | EKLKEFVKANKPELYHDIFKDESKNGYAGYIENGVKQDEFYKYLKETLSKQDGSQYFLDKIEREDF |
| | LRKQRTFDNGSIPHQLHLKEMRSILHRQGEYYPFLKENQAKIEKILTFRIPYYVGPLARKDSRFAWA |
| | EYHSDEPITPWNFDEVVDKEKSAEKFITRMTLNDLYLPEEKVLPKHSHVYETYTVYNELTKVKYV |
| | NEQGEAKFFDANMKQEIFDHVFKENRKVTKDKLMDFLGKEFDEFRIVDITGLDKENKAFNASLGT |
| | YHDLKKILDKSFLDDKENETIIEDIIQTLTLFEDRDMIRQRLQKYSDIFTPQQLKKLERRHYTGWGRL |
| | SYKLINGIRDKQTGKTILDYLIDDGYANRNFMQLINDDTLPFKEIIQKAQIIGDIDDIKAVVHDLPGSP |
| | AIKKGILQSVKIVDELVKVMGHNPDNIVIEMARENQTTNRGRTQSQQRLKKLQESLKELGSNILNE |
| | KKPSYVEDKVENTHLQNDRLFLYYIQNGKDMYTGDELDIDHLSDYDIDHIIPQAFIKDDSIDNRVLT |
| | SSAKNRGKSDDVPSIEIVRDRKADWMRLRKAGLISKRKFDNLTKAERGGLTEADKAGFIKRQLVET |
| | RQITKHVAQILDSRENTERDENDKVIREVKVITLKSNLVSQFRKDFQFYKVREINDYHHAHDAYLN |
| | AVVGTALIKKYPKLESEFVYGDYKVYDVRKMIAKSEQEIGKATAKRFFYSNIMNFFKTEIKLANGE |
| | IRKRPLIETNEETGEIVWNKEKDFATVRKVLSCPQVNIVKKTEVQTGGFSKESILPKRNSDKLIARKK |
| | DWDPKKYGGFDSPTVAYSVLVVAKVEKGKSKKLKSVKELVGITIMERSSFEKDPVDFLEAKGYKE |
| | VRKDLIIKLPKYSLFELENGRKRMLASAGELQKGNELALPSKYVNFLYLASHYEKLKGSPEDNEQK |
| | QLFVEQHKHYLDEIIEQISEFSKRVILADANLDKVLSAYNKHRDKPIREQAENIIHLFTLTNLGAPAA |
| | FKYFDTTIDRKRYTSTKEVLDSTLIHQSITGLYETRIDLSQLGGD |
| 222 | MKKPYSIGLDIGTNSVGWAVVTDDYKVPAKKMKVLGDTDKSHIKKNLLGTLLFESGETAEGRRLK |
| | RTARRRYTRRRNRILYLQEIFAEEMNKVDDSFFQRLDESFLKDDDKNFDSHPIFGNKAEEDAYHQK |
| | FPTIYHLRQYLADSPEKADLRLVYLALAHIIKYRGHELFEGDLKSENKDVQHLFNDFVEMEDKTVE |
| | GSYLSENLPNVADVLVEKVSKSRRLENVLHYFPNEKKNGLFGNFIALALGLQPNFKTNFKLSEDAK |
| | LQFSKDTYDEDLEELLGKIGDDYADLFVAAKNLYDAILLSGILTVTDPTTKAPLSASMIDRYEKHQ |
| | KDLALLKNLIKQNLTQEKYDEVFSDESKNGYAGYINGKVKQEEFYKYLKNTLSKIEGSDYFLDKIE |
| | REDFLRKQRTFDNGSIPHQIHLQEMNAIIRRQGEHYPFLKENKEKIEKILTFRIPYYVGPLARGNRDF |
| | AWLTRNSDQAIRPWNFEEIVDQASSAEDFINKMTNYDLYLPEEKVLPKHSLLYETFAVYNELTKVK |
| | FIAEGLRDYQFLDSGQKKQIVNQLFKEKRKVTEKDIIQYLHNVDGYDGIELKGIEKQFNASLSTYHD |
| | LLKIIKDKEFMDDPKNEEILENIVHTLTIFEDREMIKQRLAQYDSLFDEKVIKALTRRHYTGWGKLS |
| | AKLINGIYDKQSKKTILDYLIDDGEINRNFMQLINDDGLSFKDIIQKAQVVGKTNDVKQVVQELPGS |
| | PAIKKGILQSIKLVDELVKVMGHAPESIVIEMARENQTTARGKKNSQQRYKRIEDALKNLAPGLDS |
| | NILKENPTDNIQLQNDRLFLYYLQNGKDMYTGEALDINQLSNYDIDHIVPQAFIKDDSLDNRVLTSS |
| | KDNRGKSDNVPSIEVVQKRKAFWQQLLDSKLISERKENNLTKAERGGLDERDKVGFIKRQLVETR |
| | QITKHVAQILDARFNTEVDENNQRIRKVKIITLKSNLVSNFRKEFGLYKVREINDYHHAHDAYLNA |
| | VVAKAILKKYPKLEPEFVYGDYQKYDLKRYISRSKDPKDIEKATEKYFFYSNLLNFFKEEVHYADG |
| | TIIKRENIEYSNDTGEIAWNKEKDFAIVRKVLSCPQVNIVKKTEVQTGGFSKESILPKRNSDKLIPRK |
| | NNWDPKKYGGFDSPTVAYSVLVVAKVEKGKSKKLKPVKELVGITIMERSSFEKDPVDFLEAKGYK |
| | EVRKDLIIKLPKYSLFELENGRKRMLASAGELQKGNELALPSKYVNFLYLASHYEKLKGSPEDNEQ |
| | KQLFVEQHKHYLDEIIEQISEFSERVILADANLDKVLSAYNNHRDKPIREQAENIIHLFTLTNLGAPA |
| | AFKFFDTTIDRKRYTSTKEILDATLIHQSITGLYETRIDLSKLGGD |
| 223 | MKKSYSIGLDIGTNSVGWSVVTDDYKVPAKKMKVFGNTDKKYIKKNLLGTLLFDNGETAEGRRL |
| | KRTARRRYTRRRNRILYLQEIFAKEMSKIDESFFQRLEESFLTDDDKNFDSHPIFGNKAEEDAYHKQ |
| | FPTIYHLRKHLADSSEKADLRLVYLALAHMIKFRGHFLIEGQLKAENTNVQTLFNDFVEVYDKTVE |
| | ESHLAEITVDALSILTEKVSKSRRLENLLKYYPTEKKNTLFGNLIALSLGLQPNFKTNFKLSEDAKLQ |
| | FSKDTYEEDLEELLGKIGDEYADLFTSAKNLYDAILLSGILTVDDKSTKAPLSASMLKRYVEHHEDL |
| | EKLKDFVKANKPELYHDIFKDETKNGYAGYIENGVKQDEFYKYLKNTLSKIAGSDYFLDKIEREDF |
| | LRKQRTFDNGSIPHQVHLQEMRAILRRQGEYYPFLKENQAKIEKILTFRIPYYVGPLARKDSRFAWA |
| | EYHSDEKITPWNFDKVIDKESSARAFIEHMTNHDLYLPEEKVLPKHSPLYEKYTVYNELTKVKYVT |
| | EQGKSSFFDANMKQEIFEHVFKKNRKVTKDKFLNYLNKEFPEYRIHDLIGLDKENKSFNASLGTYH |
| | DLKKILDKSFLDNKENEEIIEDVVLTLTLFEDRDMIRQRLQKYSDIFTKDQLKKLERRHYTGWGRLS |
| | YKLINGIRDKQSNKTILDYLLEDDGVKKNINRNFMQLINDDSLSFKTIIQEAQVIGDIDDIKAVVHDL |
| | PGSPAIKKGILQSVKIVDELVKIMGHNPDNIVIEMARENQTTNRGRSQSQQRLKKLQESLKELGSNI |
| | LNEEKPSYIEDKVENSHLQNDRLFLYYIQNGKDMYTGDELDIDHLSDYDIDHIIPQAFIKDNSIDNRV |
| | LTSSAKNRGKSDDVPSLDIVRERKADWIRLYKAGLISKRKFDNLTKAERGGLTENDKAGFIKRQLV |
| | ETRQITKHVAQILDARFNTKRDENDKVIRDVKIITLKSNLVSQFRKEFGFYKIREVNDYHHAHDAYL |
| | NAVVGTALIKKYPKLESEFVYGDYKVYDVRKMIAKSEQEIGKATAKRFFYSNIMNFFKTEIKLANG |
| | EIRKRPLIETNEETGEVVWNKEKDFAIVRKVLSCPQVNIVKKTEVQTGAFTKESILPKRNSDKLIAR |
| | KKNWDPKKYGGFDSPIVAYSVLVVAKVEKGKSKKLKPVKELVGITIMEQKSFEKDPIDFLEAKGY |
| | KEVRKDLIIKLPKYSLFELENGRKRMLASAGELQKGNELALPSKYINFLYLASHYEKLKGSPEDNE |
| | QKQLFVEQHKHYLDEIIEQISEFSERVILADANLDKVLSAYNNHRDKPIREQAENIIHLFTLTNLGAP |
| | AAFKFFDTTIDRKRYTSTKEVLDATLIHQSITGLYETRIDLSQLGGD |
| 224 | MKKSYSIGLDIGTNSVGWAVVNDDYKVPAKKMRVFGDTDRSHIKKNLLGTLLFDSGETAEGRRL |
| | KRTARRRYTRRRNRILYLQEIFAEEMNKIDESFFHRLDDSFLVPEDKRGSKYPIFATLAEEKEYHKQ |
| | FPTIYHLRKKLAESNEKADLRLVYLALAHMIKYRGHFLIDDPKFKVQNNDIQGLFEKFVEEYDNVQ |
| | ETSLSKIKLNVTEILTANIPKSEKQEQLLKNYPSEKKNTLFGNLIGLALGLTPNFKTNFSLENDAKLQI |
| | SSESYEEDLGNLLALIGENFIELFSAVKNLSDGILLAGIVSDESPHAPLSTKMVIRFKEHEEDLAALK |
| | HFIKANLPEKYDEVFSDDSKNGYAGYVGVDSKVRKRNGKLATEEEFYKYLKNILNNIKGADYFLE |
| | KIEREDLLRKQRTFDNGTIPYQVHLAEMRAILHRQGDYYPFLKENREKIEKILTFRIPYYVGPLARG |
| | NRDFAWLTRNSDQAIRPWNFEEVVDQASSAEDFINKMTNYDLYLPEEKVLPKHSLLYETFAVYNE |
| | LTKVKFIAEGLRDYQFLDSGQKKQIVNQLFKEKRKVTEKDIIHYLHNVDGYDGIELKGIEKQFNASL |
| | STYHDLLKIIKDKEFMDDPKNEEILENIVHTLTIFEDREMIKQRLAQYDSLFDEKVIKALTRRHYTG |
| | WGKLSAKLINGICDKQTGNTILDYLIDDDKINRNFMQLINDDGLSFKEIIQKAQVVGKTDDVKQVV |
| | QELPGSPAIKKGILQSIKIVDELVKVMGHAPESIVIEMARENQTTARGKKNSQQRYKRIEDALKNLA |
| | PGLDSNILKEHPTDNIQLQNDRLFLYYLQNGKDMYTGESLDINQLSSYDIDHIVPQAFIKDDSLDNR |
| | VLTSSKDNRGKSDNVPSIEVVQKRKAFWQQLLDSKLISERKENNLTKAERGGLDERDKVGFIKRQL |
| | VETRQITKHVAQILDARFNTEVNEKGQKIRKVKIITLKSNLVSNFRKEFGLYKVREINDYHHAHDA |
| | YLNAVVAKAILKKYPKLEPEFVYGDYQKYDIKRYISKSKDPKEIEKATEKYFFYSNLLNFFKEEVH |
| | YADGTIIKRENIEYSKDTGEIAWNKEKDFATVRKVLSCPQVNIVKKTEVQTGGFSKESILPKRNSDK |
| | LIPRKTEWDPKKYGGFDSPIVAYSVLVVAKVEKGKSKKLKPVKELVGITIMERSSFEKDPVDFLEA |
| | KGYKEVRKDLIIKLPKYSLFELENGRKRMLASAGELQKGNELALPSKYVNFLYLASHYEKLKGSPE |
| | DNEQKQLFVEQHKHYLDEIVEQISEFSERVILADANLDKILSAYNKHRDKPIREQAENIINLFTFTNL |
| | GAPAAFKFFDTDIDRKRYTSTKEVLDATLIHQSITGLYETRIDLSQLGGD |
| 225 | MKKPYSIGLDIGTNSVGWAVVTDDYKVPAKKMKVLGDTDKSHIKKNLLGTLLFESGETAEGRRLK |
| | RTARRRYTRRRNRILYLQEIFAEEMNKVDDSFFQRLDESFLKDDDKNFDSHPIFGNKAEEDAYHQK |
| | FPTIYHLRQYLADSPEKADLRLVYLALAHIIKYRGHELFEGDLKSENKDVQHLFNDFVEMEDKTVE |
| | GSYLSENLPNVADVLVEKVSKSRRLENVLHYFPNEKKNGLFGNFIALALGLQPNFKTNFKLSEDAK |
| | LQFSKDSYDEDLEELLGKIGDDYADLFVAAKNLYDAILLSGILTVTDPTTKAPLSASMIDRYEKHH |
| | KDLALLKNLIKKNLTQEKYDEVFSDESKNGYAGYINGKVKQEEFYKYLKNTLSKIEGSDYFLDKIE |
| | REDFLRKQRTFDNGSIPHQIHLQEMNAIIRRQGEHYPFLKENKEKIEKILTFRIPYYVGPLARGNRDF |
| | AWLTRNSDQAIRPWNFEEIVDQASSAEDFINKMTNYDLYLPEEKVLPKHSLLYETFAVYNELTKVK |
| | FIAEGLRDYQFLDSGQKKQIVNQLFKEKRKVTEKDIIQYLHNVDGYDGIELKGIEKQFNASLSTYHD |
| | LLKIIKDKEFMDDPKNEEILENIVHTLTIFEDREMIKQRLAQYDSLFDEKVIKALTRRHYTGWGKLS |
| | AKLINGIYDKQSKKTILDYLIDDGEINRNFMQLINDDGLSFKDIIQKAQVVGKTNDVKQVVQELPGS |
| | PAIKKGILQSIKLVDELVKVMGHAPESIVIEMARENQTTARGKKNSQQRYKRIEDALKNLAPGLDS |
| | NILKENPTDNIQLQNDRLFLYYLQNGKDMYTGEALDINQLSNYDIDHIVPQAFIKDDSLDNRVLTSS |
| | KDNRGKSDNVPSIEVVQKRKAFWQQLLDSKLISERKFNNLTKAERGGLDERDKVGFIKRQLVETR |
| | QITKHVAQILDARFNTEVDENNQRIRKVKIITLKSNLVSNFRKEFGLYKVREINDYHHAHDAYLNA |
| | VVAKAILKKYPKLEPEFVYGDYQKYDLKRYISRSKDPKDIEKATEKYFFYSNLLNFFKEEVHYADG |
| | TIIKRENIEYSNDTGEIAWNKEKDFATVRKVLSCPQVNIVKKTEVQTGGFSKESILPKRNSDKLIARK |
| | KDWDPKKYGGFDSPTVAYSVLVIAKVEKGKSKKLKPVKELVGITIMERSSFEKNPIDFLEAKGYKE |
| | VREDLIIKLPKYSLFELENGRKRMLASAGELQKGNELALPSKYVNFLYLASHYEKLKGSPEDNEQK |
| | RLFVEQHKHYLDEIVEQISEFSERVILADANLDKILSAYNKHRDKPIREQAENIINLETLTNLGAPAA |
| | FKFFDTTIDRKRYTSTKEILDATLIHQSITGLYETRIDLSQLGGN |
| 226 | MKKPYSIGLDIGTNSVGWAVVTDDYKVPAKKMKVLGDTDKSHIKKNLLGTLLFESGETAEGRRLK |
| | RTARRRYTRRRNRILYLQEIFAEEMNKVDDSFFQRLDESFLKDDDKNFDSHPIFGNKAEEDAYHQK |
| | FPTIYHLRQYLADSPEKADLRLVYLALAHIIKYRGHELFEGDLKSENKDVQHLFNDFVEMEDKTVE |
| | GSYLSENLPNVADVLVEKVSKSRRLENVLHYFPNEKKNGLFGNFIALALGLQPNFKTNFKLSEDAK |
| | LQFSKDTYDEDLEELLGKIGDDYADLFVAAKNLYDAILLSGILTVTDPTTKAPLSASMIDRYEKHH |
| | KDLALLKNLIKQNLTQEKYDEVFSDESKNGYAGYISGKVKQEEFYKYLKNTLSKIEGSDYFLDKIE |
| | REDFLRKQRTFDNGSIPHQIHLQEMNAIIRRQGEHYPFLKENKEKIEKILTFRIPYYVGPLARGNRDF |
| | AWLTRNSDQAIRPWNFEEIVDQASSAEDFINKMTNYDLYLPEEKVLPKHSLLYETFAVYNELTKVK |
| | FIAEGLRDYQFLDSGQKKQIVNQLFKEKRKVTEKDIIQYLHNVDGYDGIELKGIEKQFNASLSTYHD |
| | LLKIIKDKEFMDDPKNEEILENIVHTLTIFEDREMIKQRLAQYDSLFDEKVIKALTRRHYTGWGKLS |
| | AKLINGIYDKQSKKTILDYLIDDGEINRNFMQLINDDGLSFKDIIQKAQVVGKTNDVKQVVQELPGS |
| | PAIKKGILQSIKLVDELVKVMGHAPESIVIEMARENQTTARGKKNSQQRYKRIEDALKNLAPGLDS |
| | NILKENPTDNIQLQNDRLFLYYLQNGKDMYTGEALDINQLSNYDIDHIVPQAFIKDDSLDNRVLTSS |
| | KDNRGKSDNVPSIEVVQKRKAFWQQLLDSKLISERKENNLTKAERGGLDERDKVGFIKRQLVETR |
| | QITKHVAQILDARFNTEVDENNQRIRKVKIITLKSNLVSNFRKEFGLYKVREINDYHHAHDAYLNA |
| | VVAKAILKKYPKLEPEFVYGDYQKYDLKRYISRSKDPKDIEKATEKYFFYSNLLNFFKEEVHYADG |
| | TIIKRENIEYSNDTGEIAWNKEKDFATVRKVLSCPQVNIVKKTEVQTGGFSKESILPKRNSDKLIARK |
| | KDWDPKKYGGFDSPTVAYSVLVVAKVEKGKSKKLKSVKELLGITIMERSSFEKNPIDFLEAKGYKE |
| | VRKDLIIKLPKYSLFELENGRKRMLASAGELQKGNELALPSKYVNFLYLASHYEKLKGSPEDNEQK |
| | QLFVEQHKHYLDEIIEQISEFSKRVILADANLDKVLSAYNKHRDKPIREQAENIIHLFTLTNLGAPAA |
| | FKYFDTTIDRKRYTSTKEVLDSTLIHQSITGLYETRIDLSQLGGD |
| 227 | MKKPYSIGLDIGTNSVGWAVITDDYKVPSKKMKVLGNTNKSHIKKNLLGTLLFDSGETAEGRRLK |
| | RTARRRYTRRRNRILYLQEIFAEEMNKVDESFFHRLDDSFLVPEDKRGSKYPIFATLQEEKEYHKQF |
| | PTIYHLRKQLAESNEKADLRLVYLALAHMIKYRGHFLIDDPKFKVQNNDIQGLFEKFVEEYDNVQE |
| | TSLSKIKLNVTEILTAKIPKSEKQEQLLKNYPSEKKNTLFGNLIGLALGLTPNFKTNFSLENDAKLQIS |
| | SESYEEDLGSLLALIGENFIELFSAVKNLSDGILLAGIVSDESPHAPLSAKMVIRFKEHEEDLAALKH |
| | FIKANLPEKYDEVFSDDSKNGYAGYVGVDSKVRKRNGKLATEEEFYKYLKDILNNVKGADYFLEK |
| | IKREDFLRKQRTFDNGTIPYQVHLAEMRAILHRQGDYYPFLKENKEKIEKILTFRIPYYVGPLARGN |
| | RDFAWLTRNSDQAIRPWNFEEIVDQASSAEDFINKMTNYDLYLPEEKVLPKHSLLYETFAVYNELT |
| | KVKFIAEGLRDYQFLDSGQKKQIVNQLFKDKRKVTEKDIIHYLHNVDGYDGIELKGIEKQFNASLS |
| | TYHDLLKIIKDKEFMDDPKNEEILENIIHTLTIFEDREMIKQRLAQYDSLFDEKVIKALTRRHYTGWG |
| | KLSAKLINGIYDKQSKKTILDYLIDDDKINRNFMQLINDDGLSFKEIIQKAQVVGKTDDVKQVVQEL |
| | PGSPAIKKGILQSIKIVDELVKVMGHAPESIVIEMARENQTTARGKKNSQQRYKRIEDALKNLAPGL |
| | DSNILKENPTDNIQLQNDRLFLYYLQNGKDMYTGEPLDINQLSSYDIDHIVPQAFIKDDSLDNRVLT |
| | SSKDNRGKSDNVPSIEVVQKRKAFWQQLLDSKLISERKENNLTKAERGGLDERDKVGFIRRQLVET |
| | RQITKHVAQILDARFNTEVNEKNQRIRKVKIITLKSNLVSNFRKEFGLYKVREINDYHHAHDAYLN |
| | AVVAKAILKKYPKLEPEFVYGDYQKYDIKRYISRSKDPKDIEKATEKYFFYSNLLNFFKEEVHYAD |
| | GTIIKRENIEYSKDTGEIAWNKEKDFATVRKVLSCPQVNIVKKTEVQTGGFSKESILPKRNSDKLIAR |
| | KKDWDPKKYGGFDSPTVAYSVLVVAKVEKGKSKKLKSVKELVGITIMERSSFEKDPVDFLEAKGY |
| | KEVRKDLIIKLPKYSLFELENGRKRMLASAGELQKGNELALPSKYVNFLYLASHYEKLKGSPEDNE |
| | QKQLFVEQHKHYLDEIIEQISEFSKRVILADANLDKVLSAYNKHRDKPIREQAENIIHLFTLTNLGAP |
| | AAFKYFDTTIDRKRYTSTKEVLDATLIHQSITGLYETRIDLSQLGGD |
| 228 | MKKSYSIGLDIGTNSVGWAVVNDDYKVPAKKMRVFGDTDRSHIKKNLLGTLLFDSGETAEGRRL |
| | KRTARRRYTRRRNRILYLQEIFAEEMNKIDESFFHRLDDSFLVPEDKRGSKYPIFATLAEEKEYHKQ |
| | FPTIYHLRKKLAESNEKADLRLVYLALAHMIKYRGHFLIDDPKFKVQNNDIQGLFEKFVEEYDNVQ |
| | ETSLSKIKLNVTEILTANIPKSEKQEQLLKNYPSEKKNTLFGNLIGLALGLTPNFKTNFSLENDAKLQI |
| | SSESYEEDLGNLLALIGENFIELFSAVKNLSDGILLAGIVSDESPHAPLSTKMVIRFKEHEEDLAALK |
| | HFIKANLPEKYDEVFSDDSKNGYAGYVGVDSKVRKRNGKLATEEEFYKYLKNILNNIKGADYFLE |
| | KIEREDLLRKQRTFDNGTIPYQVHLAEMRAILHRQGDYYPFLKENREKIEKILTFRIPYYVGPLARG |
| | NRDFAWLTRNSDQAIRPWNFEEVVDQASSAEDFINKMTNYDLYLPEEKVLPKHSLLYETFAVYNE |
| | LTKVKFIAEGLRDYQFLDSGQKKQIVNQLFKEKRKVTEKDIIHYLHNVDGYDGIELKGIEKQFNASL |
| | STYHDLLKIIKDKEFMDDPKNEEILENIVHTLTIFEDREMIKQRLAQYDSLFDEKVIKALTRRHYTG |
| | WGKLSAKLINGICDKQTGNTILDYLIDDDKINRNFMQLINDDGLSFKEIIQKAQVVGKTDDVKQVV |
| | QELPGSPAIKKGILQSIKIVDELVKVMGHAPESIVIEMARENQTTARGKKNSQQRYKRIEDALKNLA |
| | PGLDSNILKEHPTDNIQLQNDRLFLYYLQNGKDMYTGESLDINQLSSYDIDHIVPQAFIKDDSLDNR |
| | VLTSSKDNRGKSDNVPSIEVVQKRKAFWQQLLDSKLISERKENNLTKAERGGLDERDKVGFIKRQL |
| | VETRQITKHVAQILDARFNTEVNEKGQKIRKVKIITLKSNLVSNFRKEFGLYKVREINDYHHAHDA |
| | YLNAVVAKAILKKYPKLEPEFVYGDYQKYDIKRYISKSKDPKEIEKATEKYFFYSNLLNFFKEEVH |
| | YADGTIIKRENIEYSKDTGEIAWNKEKDFATVRKVLSCPQVNIVKKTEVQTGGFSKESILPKRNSDK |
| | LIARKKDWDPKKYGGFDSPTVAYSVLVVAKVEKGKSKKLKSVKELVGITIMERSSFEKDPVDFLE |
| | AKGYKEVRKDLIIKLPKYSLFELENGRKRMLASAGELQKGNELALPSKYVNFLYLASHYEKLKGSP |
| | EDNEQKQLFVEQHKHYLDEIIEQISEFSKRVILADANLDKVLSAYNKHRDKPIREQAENIIHLFTLTN |
| | LGAPAAFKYFDTTIDRKRYTSTKEVLDATLIHQSITGLYETRIDLSQLGGD |
| 229 | MKKPYSIGLDIGTNSVGWAVITDDYKVPSKKMKVLGNTDKSHIKKNLIGALLFDSGNTAEDRRLK |
| | RTARRRYTRRRNRILYLQEIFATEMNKVDESFFHRLDDSFLVPEDKRGSKYPIFGTLEEEKEYHKQF |
| | PTIYHLRKQLAESNEKADLRLVYLALAHMIKYRGHFLIDDPKFKVQNNDIQGLFEKFVEEFDNVQE |
| | TSLSKIKLNVTEILTANIPKSEKQEQLLKNYPSEKKNTLFGNLIGLALGLTPNFKTNFSLENDAKLQIS |
| | SESYEEDLGNLLALIGENFIELFSAVKNLSDGILLAGIVSDESPHAPLSTKMVIRFKEHEEDLAALKH |
| | FIKANLPEKYDEVFSDDSKNGYAGYVGVDSKVRKRNGKLATEEEFYKYLKDILNNVKGADYFLEK |
| | IKREDFLRKQRTFDNGTIPYQVHLTEMKAILHRQGEYYPFLKENKEKIQQILTFRIPYYVGPLARGN |
| | RDFAWLTRNSDQAIRPWNFEEVVDQASSAEDFINKMTNYDLYLPEEKVLPKHSLLYETFAVYNEL |
| | TKVKFIAEGLRDYQFLDSGQKKQIVNQLFKEKRKVTEKDIIQFLHNVDGYDGIELKGIEKQFNASLS |
| | TYHDLLKIIKDKEFMDDAKNEEILENIVHTLTIFEDREMIKQRLAQYDSLFDEKVIKALARRHYTGW |
| | GKLSAKLINGICDKQTGDTILDYLIDDDKINRNFMQLIHDDGLSFKEIIQKAQVFGKTDDVKQVVQE |
| | LPGSPAIKKGILQSIKIVDELVKVMGHAPESIVIEMARENQTTARGKKNSQQRYKRIEDSLKILASGL |
| | NSKILKEHPTDNQALQNDRLFLYYLQNGKDMYTGEALDINQLSSYDIDHIIPQAFIKDDSLDNRVLT |
| | SSKDNRGKSDNVPSIEVVQRRKAFWQQLLDSKLISERKENNLTKAERGGLDERDKVGFIKRQLVET |
| | RQITKHVAQILDARFNTEVNEKNQKIRTVKIITLKSNLVSNFRKEFGLYKVREINDYHHAHDAYLN |
| | AVVAKAILKKYPKLEPEFVYGDYQKYDLKRYISRSKDPKEVEKATEKYFFYSNLLNFFKEEVHYA |
| | DGTIVKRENIEYSKDTGEIAWNKEKDFATVRKVLSCPQVNIVKKTEVQTGGFSKESILPKRNSDKLI |
| | ARKKDWDPKKYGGFDSPTVAYSVLVVAKVEKGKSKKLKSVKELVGITIMERSSFEKDPVDFLEAK |
| | GYKEVRKDLIIKLPKYSLFELENGRKRMLASAGELQKGNELALPSKYVNFLYLASHYEKLKGSPED |
| | NEQKQLFVEQHKHYLDEIIEQISEFSKRVILADANLDKVLSAYNKHRDKPIREQAENIIHLFTLTNLG |
| | APAAFKYFDTTIDRKRYTSTKEVLDATLIHQSITGLYETRIDLSQLGGD |
| 230 | MKKSYSIGLDIGTNSVGWSVITDDYKVPAKKMKVLGNTDKKYIKKNLLGALLFDSGETAEATRLK |
| | RTARRRYTRRKNRLRYLQEIFAKEMAKVDESFFQRLEESFLTDDDKNFDSHPIFGNKAEEDAYHDE |
| | FPTIYHLRQYLADSPQKADLRLVYLALAHMIKFRGHFLIEGQLNAENTDVQTLFNDFVEVYDKTVE |
| | ESHLSEITVDALSILTEKVSKSRRLENLVKCYPTEKKNTLFGNLIALSLGLQPNFKTNFKLSEDAKLQ |
| | FSKDTYEEDLEELLGKIGDEYADLFASAKNLYDAILLSGILTVDDNTTKAPLSASMVKRYAEHHED |
| | LEKLKEFVKANKPELYHDIFKDETKNGYAGYIENGVKQDEFYKYLKNTLSKIAGSDYFLDKIERED |
| | FLRKQRTFDNGSIPHQIHLQEMHAILRRQGEYYPFLKENQAKIEKILTFRIPYYVGPLARKNSRFAW |
| | AEYHSDEKITPWNFEEIVDKEESATAFIHRMTNNDLYLPEEKVLPKHSLIYEEFTVYNELTKVKYVT |
| | DQGKAYYFDANMKQEIFDGVFKEHRKVTKDKLMNFLEKEFDEFRIVDLTGLDKENKSFNASLGTY |
| | HDLKKILDKSFLDDKTNETIIEDIIQTLTLFEDRDMIRQRLQKYSDIFTPQQLKKLERRHYTGWGRLS |
| | YKLINGIRNKENGKTILDYLIDDGYANRNFMQLINDDTLPFKQIIQEAQIIGDIDDIETVVHDLPGSPA |
| | IKKGILQSVKIVDELVKVMGHNPDNIVIEMARENQTTNRGRSQSQQRLKKLQESLKELGSNILNEEK |
| | PSYVEDKVENSHLQNDKLFLYYIQNGKDMYTGEELDIDHLSDYDIDHIIPQAFIKDNSIDNRVLTSS |
| | AKNRGKSDDVPSLDIVHDRKADWIRLYKSGLISKRKFDNLTKAEHGGLTEDDKAGFIKRQLVETR |
| | QITKHVAQILDSRFNTERDENDKVIRDVKVITLKSNLVSQFRKDFELHKVREINDYHHAHDAYLNA |
| | VIGNALLVKYPQLEPEFVYGKFKHFHGHKENKATAKKFFYSNIMALFKKDVEYSDDTGEMVWNK |
| | EKDFATVRKVLSCPQVNIVKKTEVQTGGFSKESILPKRNSDKLIARKKDWDPKKYGGFDSPTVAYS |
| | VLVVAKVEKGKSKKLKSVKELVGITIMERSSFEKNPIDFLEAKGYKEVRKDLIIKLPKYSLFELENG |
| | RKRMLASAGELQKGNELALPSKYVNFLYLASHYEKLKGSPEDNEQKQLFVEQHKHYLDEIIEQISE |
| | FSKRVILADANLDKVLSAYNKHRDKPIREQAENIIHLFTLTNLGAPAAFKYFDTTIDRKRYTSTKEV |
| | LDATLIHQSITGLYETRIDLSQLGGD |
| 231 | MKKPYSIGLDIGTNSVGWAVVTDDYKVPAKKMKVLGNTDKSHIKKNLLGALLFDSGETAEGRRL |
| | KRTARRRYTRRRNRILYLQEIFAEEMNKIDESFFHRLDDSFLVPEDKRGSKYPIFATLEEEKEYHKK |
| | FPTIYHLRKHLAESNEKADLRLVYLALAHMIKYRGHFLIDDPKFKVQNNDIQGLFEKFVEEYDNVQ |
| | ETSLSKIKLNVTEILTANIPKSEKQEQLLKNYPSEKKNTLFGNLIGLALGLTPNFKTNFSLENDAKLQI |
| | SSESYEEDLGNLLALIGENFIELFSAVKNLSDGILLAGIVSDESPHAPLSTKMVIRFKEHEEDLAALK |
| | HFIKANLPEKYDEVFSDDSKNGYAGYVGVDSKVRKRNGKLATEEEFYKYLKDILNNVKGADYFLE |
| | KIKREDLLRKQRTFDNGTIPYQVHLSEMKAIIHRQGEHYPFLKENKEKIEKILTFRIPYYVGPLARGN |
| | RDFAWLTRNSDQAIRPWNFEEIVDKASSAEDFINKMTNYDLYLPEEKVLPKHSLLYETFAVYNELT |
| | KVKFIAEGLRDYQFLDSGQKKQIVNQLFKEKRKVTEKDIIHYLHNVDGYDGIELKGIEKQFNASLST |
| | YHDLLKIIKDKEFMDDPKNEEILENIVHTLTIFEDREMIKQRLAQYDSLFDEKVIKALTRRHYTGWG |
| | KLSAKLINGIYDKQSKKTILDYLIDDGEINRNFMQLIHDDGLSFKEIIQKAQVFGKTDDVKQVVQEL |
| | PGSPAIKKGILQSIKIVDELVKVMGHAPESIVIEMARENQTTARGKKNSQQRYKRIEDSLKNLASGL |
| | DSNILKENPTDNVQLQNDRLFLYYLQNGKDMYTGEALDINQLSNYDIDHIIPQAFIKDDSLDNRVLT |
| | SSKDNRGKSDNVPSIEVVEKMKAFWQQLLDSKLISERKYNNLTKAERERGGLNELDKVGFIKRQL |
| | VETRQITKHVAQILDARFNTEVNENNQKIRKVKIITLKSNLVSNFRKEFGLYKVREINDYHHAHDA |
| | YLNAVVAKAILKKYPKLEPEFVYGDYQKYDIKRYISRSKDPKDIEKATEKYFFYSNLLNFFKEEVH |
| | YADGTIIKRENIEYSKDTGEIAWNKEKDFATVRKVLSCPQVNIVKKTEVQTGGFSKESILPKRNSDK |
| | LIPRKTEWDPKKYGGFDSPIVAYSVLVVAKVEKGKSKKLKPVKELVGITIMERSSFEKDPVDFLEA |
| | KGYKEVRKDLIIKLPKYSLFELENGRKRMLASAGELQKGNELALPSKYVNFLYLASHYEKLKGSPE |
| | DNEQKQLFVEQHKHYLDEIVEQISEFSERVILADANLDKILSAYNKHRDKPIREQAENIINLFTFTNL |
| | GAPAAFKFFDTDIDRKRYTSTKEVLDATLIHQSITGLYETRIDLSQLGGD |
| 232 | MKKPYSIGLDIGTNSVGWAVITDDYKVPAKKMKVLGNTDKSHIKKNLLGALLFDSGNTAEDRRLK |
| | RTARRRYTRRRNRILYLQEIFAEEMNKIDESFFHRLDDSFLVPEDKRGSKYPIFATLEEEKEYHKQFP |
| | TIYHLRKKLAESNEKADLRLIYLALAHMIKYRGHFLIDDPKFKVQNNDIQGLFEKFVEEYDNVQET |
| | SLSKIKLNVTEILTANIPKSEKQEQLLKNYPSEKKNTLFGNLIGLALGLTPNFKTNFSLENDAKLQISS |
| | ESYEEDLGNLLALIGENFIELFSAVKNLSDGILLAGIVSDESPHAPLSTKMVIRFKEHEEDLAALKHFI |
| | KANLPEKYDEVFSDDSKNGYAGYVGVDSKVRKRNGKLATEEEFYKYLKEILNNVKGADYFLEKI |
| | KREDFLRKQRTFDNGTIPYQVHLAEMRAILHRQGDYYPFLKDNKEKIEKILTFRIPYYVGPLARGNR |
| | DFAWLTRNSDQAIRPWNFEEVVDKASSAEDFINKMTNYDLYLPEEKVLPKHSLLYETFAVYNELT |
| | KVKFIAEGLRDYQFLDSGQKKQIVNQLFKEKRKVTEKDIIQFLHNVDGYDGIELKGIEKQFNASLST |
| | YHDLLKIIKDKEFMDDPKNEEILENIVHTLTIFEDREMIKQRLAQYDSLFDEKVIKALTRRHYTGWG |
| | KLSAKLINGIRDKQSNKTILDYLIDDDKINRNFMQLINDDGLSFKDIIQKAQVVGKTDDVKQVVQEL |
| | PGSPAIKKGILQSIKIVDELVKVMGHAPESIVIEMARENQTTARGKKNSQQRYKRIEDALKNLAPGL |
| | DSNILKENPTDNIQLQNDRLFLYYLQNGKDMYTGESLDINQLSNYDIDHIVPQAFIKDDSLDNRVLT |
| | SSKDNRGKSDNVPSLEVVQKRKAFWQQLLDSKLISERKENNLTKAERGGLDERDKVGFIKRQLVE |
| | TRQITKHVAQILDARFNTEVNEKNQKIRKVKIITLKSNLVSNFRKEFRLYKVREINDYHHAHDAYL |
| | NAVVAKAILKKYPKLEPEFVYGDYQKYDIKRYISKSKDPKDIEKATQKYFFYSNLLNFFKEEVHYA |
| | DGTIVKRENVEYSKDTGEIAWNKEKDFATVRKVLSCPQVNIVKKTEVQTGGFSKESILPKRNSDKLI |
| | ARKKDWDPKKYGGFDSPTVAYSVLVVAKVEKGKSKKLKSVKELLGITIMERSSFEKNPIDFLEAK |
| | GYKEVRKDLIIKLPKYSLFELENGRKRMLASAGELQKGNELALPSKYVNFLYLASHYEKLKGSPED |
| | NEQKQLFVEQHKHYLDEIIEQISEFSKRVILADANLDKVLSAYNKHRDKPIREQAENIIHLFTLTNLG |
| | APAAFKYFDTTIDRKRYTSTKEVLDATLIHQSITGLYETRIDLSQLGGD |
| 233 | MKKSYSIGLDIGTNSVGWSVITDDYKVPAKKMKVLGNTDKKYIKKNLLGALLFDSGETAEATRLK |
| | RTARRRYTRRKNRLRYLQEIFAKEMAKVDESFFQRLEESFLTDDDKNFDSHPIFGNKAEEDAYHDE |
| | FPTIYHLRQYLADSPQKADLRLVYLALAHMIKFRGHFLIEGQLNAENTDVQTLFNDFVEVYDKTVE |
| | ESHLSEITVDALSILTEKVSKSRRLENLVKCYPTEKKNTLFGNLIALSLGLQPNFKTNFKLSEDAKLQ |
| | FSKDTYEEDLEELLGKIGDEYADLFASAKNLYDAILLSGILTVDDNTTKAPLSASMVKRYAEHHED |
| | LEKLKEFVKANKPELYHDIFKDETKNGYAGYIENGVKQDEFYKYLKNTLSKIAGSDYFLDKIERED |
| | FLRKQRTFDNGSIPHQIHLQEMHAILRRQGEYYPFLKENQAKIEKILTFRIPYYVGPLARKNSRFAW |
| | AEYHSDEKITPWNFEEIVDKEESATAFIHRMTNNDLYLPEEKVLPKHSLIYEEFTVYNELTKVKYVT |
| | DQGKAYYFDANMKQEIFDGVFKEHRKVTKDKLMNFLEKEFDEFRIVDLTGLDKENKSFNASLGTY |
| | HDLKKILDKSFLDDKTNETIIEDIIQTLTLFEDRDMIRQRLQKYSDIFTPQQLKKLERRHYTGWGRLS |
| | YKLINGIRNKENGKTILDYLIDDGYANRNFMQLINDDTLPFKQIIQEAQIIGDIDDIETVVHDLPGSPA |
| | IKKGILQSVKIVDELVKVMGHNPDNIVIEMARENQTTNRGRSQSQQRLKKLQESLKELGSNILNEEK |
| | PSYVEDKVENSHLQNDKLFLYYIQNGKDMYTGEELDIDHLSDYDIDHIIPQAFIKDNSIDNRVLTSS |
| | AKNRGKSDDVPSLDIVHDRKADWIRLYKSGLISKRKFDNLTKAEHGGLTEDDKAGFIKRQLVETR |
| | QITKHVAQILDSRENTERDENDKVIRDVKVITLKSNLVSQFRKDFELHKVREINDYHHAHDAYLNA |
| | VIGNALLVKYPQLEPEFVYGKFKHFHGHKENKATAKKFFYSNIMALFKKDVEYSDDTGEMVWNK |
| | EKDFATVRKVLSCPQVNIVKKTEVQTGGFSKESILPKRNSDKLIARKKDWDPKKYGGFDSPTVAYS |
| | VLVVAKVEKGKSKKLKSVKELVGITIMERSSFEKDPVDFLEAKGYKEVRKDLIIKLPKYSLFELENG |
| | RKRMLASAGELQKGNELALPSKYVNFLYLASHYEKLKGSPEDNEQKQLFVEQHQHYLDEIIEQISE |
| | FSKRVILADANLDKVLSAYNKHRDKPIREQAENIIHLFTLTNLGAPAAFKYFDTTIDRKRYTSTKEV |
| | LDATLIHQSITGLYETRIDLSQLGGD |
| 234 | MNKPYSIGLDIGTNSVGWSVVDDDYKVPAKKMRVFGDTDKGHIKKNLLGTLLFDSGETAESRRLK |
| | RTARRRYTRRRNRILYLQEIFAKEMAKVDESFFHRLDESFLTDDDKTFDSHPIFGNKAEEDAYHQQ |
| | FPTIYHLRKHLADSTEKADLRLVYLALAHMIKFRGHFLIEGELNAENTDVQKLFADFVGVYDRTFD |
| | DSHLSEITVDASSILTEKISKSRRLEKLIKHYPTEKKNTLFGNLVALALGLQPNFKTNFKLSEDAKLQ |
| | FSKDTYEEDLEELLGKIGDDYADLFTSAKNLYDAILLSGILTVDDESTKAPLSASMIKRYAEHHEDL |
| | EKLKEFVKANKPELYHDIFKDESKNGYAGYIENGVKQDEFYKYLKETLSKQDGSQYFLDKIEREDF |
| | LRKQRTFDNGSIPHQLHLKEMRSILHRQGEYYPFLKENQAKIEKILTFRIPYYVGPLARKDSRFAWA |
| | EYHSDEPITPWNFDEVVDKEKSAEKFITRMTLNDLYLPEEKVLPKHSHVYETYTVYNELTKVKYV |
| | NEQGEAKFFDANMKQEIFDHVFKENRKVTKDKLMDFLGKEFDEFRIVDITGLDKENKAFNASLGT |
| | YHDLKKILDKSFLDDKENETIIEDIIQTLTLFEDRDMIRQRLQKYSDIFTPQQLKKLERRHYTGWGRL |
| | SYKLINGIRDKQTGKTILDYLIDDGYANRNFMQLINDDTLPFKEIIQKAQIIGDIDDIKAVVHDLPGSP |
| | AIKKGILQSVKIVDELVKVMGHNPDNIVIEMARENQTTNRGRTQSQQRLKKLQESLKELGSNILNE |
| | KKPSYVEDKVENTHLQNDRLFLYYIQNGKDMYTGDELDIDHLSDYDIDHIIPQAFIKDDSIDNRVLT |
| | SSAKNRGKSDDVPSIEIVRDRKADWMRLRKAGLISKRKFDNLTKAERGGLTEADKAGFIKRQLVET |
| | RQITKHVAQILDSRENTERDENDKVIREVKVITLKSNLVSQFRKDFQFYKVREINDYHHAHDAYLN |
| | AVVGTALIKKYPKLESEFVYGDYKVYDVRKMIAKSEQEIGKATAKRFFYSNIMNFFKTEIKLANGE |
| | IRKRPLIETNEETGEIVWNKEKDFATVRKVLSCPQVNIVKKTEVQTGGFSKESILPKRNSDKLIARKK |
| | DWDPKKYGGFDSPTVAYSVLVVAKVEKGKSKKLKSVKELVGITIMERSSFEKDPVDFLEAKGYKE |
| | VRKDLIIKLPKYSLFELENGRKRMLASAGELQKGNELALPSKYVNFLYLASHYEKLKGSPEDNEQK |
| | QLFVEQHKHYLDEIIEQISEFSKRVILADANLDKVLSAYNKHRDKPIREQAENIIHLFTLTNLGAPAA |
| | FKYFDTTIDRKRYTSTKEVLDATLIHQSITGLYETRIDLSQLGGD |
| 235 | MNKPYSIGLDIGTNSVGWSVVDDDYKVPAKKMRVFGDTDKGHIKKNLLGTLLFDSGETAESRRLK |
| | RTARRRYTRRRNRILYLQEIFAKEMAKVDESFFHRLDESFLTDDDKTFDSHPIFGNKAEEDAYHQQ |
| | FPTIYHLRKHLADSTEKADLRLVYLALAHMIKFRGHFLIEGELNAENTDVQKLFADFVGVYDRTFD |
| | DSHLSEITVDASSILTEKISKSRRLEKLIKHYPTEKKNTLFGNLVALALGLQPNFKTNFKLSEDAKLQ |
| | FSKDTYEEDLEELLGKIGDDYADLFTSAKNLYDAILLSGILTVDDESTKAPLSASMIKRYAEHHEDL |
| | EKLKEFVKANKPELYHDIFKDESKNGYAGYIENGVKQDEFYKYLKETLSKQDGSQYFLDKIEREDF |
| | LRKQRTFDNGSIPHQLHLKEMRSILHRQGEYYPFLKENQAKIEKILTFRIPYYVGPLARKDSRFAWA |
| | EYHSDEPITPWNFDEVVDKEKSAEKFITRMTLNDLYLPEEKVLPKHSHVYETYTVYNELTKVKYV |
| | NEQGEAKFFDANMKQEIFDHVFKENRKVTKDKLMDFLGKEFDEFRIVDITGLDKENKAFNASLGT |
| | YHDLKKILDKSFLDDKENETIIEDIIQTLTLFEDRDMIRQRLQKYSDIFTPQQLKKLERRHYTGWGRL |
| | SYKLINGIRDKQTGKTILDYLIDDGYANRNFMQLINDDTLPFKEIIQKAQIIGDIDDIKAVVHDLPGSP |
| | AIKKGILQSVKIVDELVKVMGHNPDNIVIEMARENQTTNRGRTQSQQRLKKLQESLKELGSNILNE |
| | KKPSYVEDKVENTHLQNDRLFLYYIQNGKDMYTGDELDIDHLSDYDIDHIIPQAFIKDDSIDNRVLT |
| | SSAKNRGKSDDVPSIEIVRDRKADWMRLRKAGLISKRKFDNLTKAERGGLTEADKAGFIKRQLVET |
| | RQITKHVAQILDSRENTERDENDKVIREVKVITLKSNLVSQFRKDFQFYKVREINDYHHAHDAYLN |
| | AVVGTALIKKYPKLESEFVYGDYKVYDVRKMIAKSEQEIGKATAKRFFYSNIMNFFKTEIKLANGE |
| | IRKRPLIETNEETGEIVWNKEKDFATVRKVLSCPQVNIVKKTEVQTGGFSKESILPKRNSDKLIARKK |
| | DWDPKKYGGFDSPTVAYSVLVVAKVEKGKSKKLKSVKELVGITIMERSSFEKDPVDFLEAKGYKE |
| | VRKDLIIKLPKYSLFELENGRKRMLASAGELQKGNELALPSKYVNFLYLASHYEKLKGSPEDNEQK |
| | QLFVEQHQHYLDEIIEQISEFSKRVILADANLDKVLSAYNKHRDKPIREQAENIIHLFTLTNLGAPAA |
| | FKYFDTTIDRKRYTSTKEVLDATLIHQSITGLYETRIDLSQLGGD |
| 236 | MKKPYSIGLDIGTNSVGWAVVTDDYKVPAKKMKVLGNTDKSHIKKNLLGALLFEGGETAEGRRL |
| | KRTARRRYTRRRNRILYLQEIFATEMNKVDESFFHRLDDSFLVPEDKRGSKYPIFGTLEEEKEYHKQ |
| | FPTIYHLRKQLAESNEKADLRLVYLALAHMIKYRGHFLIDDPKFKVQNNDIQGLFEKFVEEYDNVQ |
| | ETSLSKIKLNVTEILTANIPKSEKQEQLLKNYPSEKKNTLFGNLIGLALGLTPNFKTNFSLENDAKLQI |
| | SSESYEEDLGNLLALIGENFIELFSAVKNLSDGILLAGIVSDESPHAPLSTKMVIRFKEHEEDLAALK |
| | HFIKANLPEKYDEVFSDDSKNGYAGYVGVDSKVRKRNGKLATEEEFYKYLKDILNNVKGADYFLE |
| | KIKREDFLRKQRTFDNGTIPYQVHLQEMKAILHRQGEHYPFLKENKEKIEKILTFRIPYYVGPLARG |
| | NRDFAWLTRNSDQAIRPWNFEEIVDKASSAEDFINKMTNYDLYLPEEKVLPKHSLLYETFAVYNEL |
| | TKVKFIAEGLRDYQFLDSGQKKQIVNQLFKEKRKVTEKDIIHYLHNVDGYDGIELKGIEKQFNASLS |
| | TYHDLLKIIKDKEFMDDPKNEEILENIVHTLTIFEDREMIKQRLAQYDSLFDEKVIKALTRRHYTGW |
| | GKLSAKLINGIYDKQSQKTILDYLIDDDKINRNFMQLINDDGLSFKEIIQKAQVVGKTDDVKQVVQ |
| | ELPGSPAIKKGILQSIKIVDELVKVMGHAPESIVIEMARENQTTARGKKNSQQRYKRIEDALKNLAP |
| | GLDSNILKENPTDNVQLQNDRLFLYYLQNGRDMYTGKPLDINQLSNYDIDHIIPQAFIKDDSLDNRV |
| | LTSSKDNRGKSDNVPSIEVVEKMKAFWQQLLDSKLISERKYNNLTKAERERGGLNELDKVGFIKR |
| | QLVETRQITKHVAQILDARFNTEVNENNQKIRKVKIITLKSNLVSNFRKEFGLYKVREINDYHHAHD |
| | AYLNAVVAKAILKKYPKLEPEFVYGDYQKYDLKRYISRSKDPKDIEKATEKYFFYSNLLNFFKEEV |
| | HYADGTIIKRENIEYSKDTGEIAWNKEKDFATVRKVLSCPQVNIVKKTEVQTGGFSKESILPKRNSD |
| | KLIPRKTEWDPKKYGGFDSPTVAYSVLVVAKVEKGKSKKLKSVKELVGITIMERSSFEKDPVDFLE |
| | AKGYKEVRKDLIIKLPKYSLFELENGRRRLLASAGELQKGNELALPSKYVNFLYLASHYEKLKGSP |
| | EDNEQKQLFVEQHKHYLDEIVEQISEFSERVILADANLDKVLSAYNKNRDRPIREQAENIIHLFTLTN |
| | LGAPAAFKFFDTTIDRKRYTSTKEVLDATLIHQSITGLYETRIDLSQLGGD |
| 237 | MKKPYSIGLDIGTNSVGWAVVTDDYKVPAKKMKVLGDTDKSHIKKNLLGTLLFESGETAEGRRLK |
| | RTARRRYTRRRNRILYLQEIFAEEMNKVDDSFFQRLDESFLKDDDKNFDSHPIFGNKAEEDAYHQK |
| | FPTIYHLRQYLADSPEKADLRLVYLALAHIIKYRGHELFEGDLKSENKDVQHLFNDFVEMFDKTVE |
| | GSYLSENLPNVADVLVEKVSKSRRLENVLHYFPNEKKNGLFGNFIALALGLQPNFKTNFKLSEDAK |
| | LQFSKDTYDEDLEELLGKIGDDYADLFVAAKNLYDAILLSGILTVTDPTTKAPLSASMIDRYEKHH |
| | KDLALLKNLIKQNLTQEKYDEVFSDESKNGYAGYISGKVKQEEFYKYLKNTLSKIEGSDYFLDKIE |
| | REDFLRKQRTFDNGSIPHQIHLQEMNAIIRRQGEHYPFLKENKEKIEKILTFRIPYYVGPLARGNRDF |
| | AWLTRNSDQAIRPWNFEEIVDQASSAEDFINKMTNYDLYLPEEKVLPKHSLLYETFAVYNELTKVK |
| | FIAEGLRDYQFLDSGQKKQIVNQLFKEKRKVTEKDIIQYLHNVDGYDGIELKGIEKQFNASLSTYHD |
| | LLKIIKDKEFMDDPKNEEILENIVHTLTIFEDREMIKQRLAQYDSLFDEKVIKALTRRHYTGWGKLS |
| | AKLINGIYDKQSKKTILDYLIDDGEINRNFMQLINDDGLSFKDIIQKAQVVGKTNDVKQVVQELPGS |
| | PAIKKGILQSIKLVDELVKVMGHAPESIVIEMARENQTTARGKKNSQQRYKRIEDALKNLAPGLDS |
| | NILKENPTDNIQLQNDRLFLYYLQNGKDMYTGEALDINQLSNYDIDHIVPQAFIKDDSLDNRVLTSS |
| | KDNRGKSDNVPSIEVVQKRKAFWQQLLDSKLISERKENNLTKAERGGLDERDKVGFIKRQLVETR |
| | QITKHVAQILDARFNTEVDENNQRIRKVKIITLKSNLVSNFRKEFGLYKVREINDYHHAHDAYLNA |
| | VVAKAILKKYPKLEPEFVYGDYQKYDLKRYISRSKDPKDIEKATEKYFFYSNLLNFFKEEVHYADG |
| | TIIKRENIEYSNDTGEIAWNKEKDFATVRKVLSCPQVNIVKKTEVQTGGFSKESILPKRNSDKLIARK |
| | KDWDPKKYGGFDSPTVAYSVLVVAKVEKGKSKKLKSVKELVGITIMERSSFEKDPIDFLEAKGYK |
| | EVRKDLIIKLPKYSLFELENGRKRMLASAGELQKGNELALPSKYVNFLYLASHYEKLKGSPEDNEQ |
| | KQLFVEQHKHYLDEIIEQISEFSKRVILADANLDKVLSAYNKHRDKPIREQAENIIHLFTLTNLGAPA |
| | AFKYFDTTIDRKRYTSTKEVLDATLIHQSITGLYETRIDLSQLGGD |
| 238 | MKKSYTIGLDIGTNSVGWSVITDDYKVPAKKMKVLGNTDKKYIKKNLLGALLFDSGETAEATRLK |
| | RTARRRYTRRKNRLRYLQEIFAKEMAKVDESFFQRLEESFLTDDDKNFDSHPIFGNKAEEDTYHDE |
| | FPTIYHLRQYLADSPQKADLRLVYLALAHMIKFRGHFLIEGQLNAENTNVQTLFNDFVEVYDKTVE |
| | ESHLSEITVDALSILTEKVSKSRRLENLVKCYPTEKKNTLFGNLIALSLGLQPNFKTNFKLSEDAKLQ |
| | FSKDTYEEDLEELLGKIGDEYADLFASAKNLYDAILLSGILTVDDNSTKAPLSASMVKRYAEHHED |
| | LEKLKEFVKANKPELYHDIFKDETKNGYAGYIENGVKQDEFYKYLKNTLSKIAGSDYFLDKIERED |
| | FLRKQRTFDNGSIPHQIHLQEMHAILRRQGEHYPFLKENQAKIEKILTFRIPYYVGPLARKNSRFAW |
| | AEYHSDEKITPWNFEEIVDKEESATAFIHRMTNNDLYLPEEKVLPKHSLIYEEFTVYNELTKVKYVT |
| | DQGKAYYFDANMKQEIFDGVFKEHRKVTKDKLMNFLEKEFDEFRIVDLTGLDKENKSFNASLGTY |
| | HDLKKILDKSFLDDKTNETIIEDIIQTLTLFEDRDMIRQRLQKYSDIFTPQQLKKLERRHYTGWGRLS |
| | YKLINGIRNKENGKTILDYLIDDGYANRNFMQLINDDTLPFKQIIQEAQIIGDIDDIETVVHDLPGSPA |
| | IKKGILQSVKIVDELVKVMGHNPDNIVIEMARENQTTNRGRSQSQQRLKKLQESLKELGSNILNEEK |
| | PSYVEDKVENSHLQNDKLFLYYIQNGKDMYTGEELDIDHLSDYDIDHIIPQAFIKDNSIDNRVLTSS |
| | AKNRGKSDDVPSLDIVHDRKADWIRLYKSGLISKRKFDNLTKAEHGGLTEDDKAGFIKRQLVETR |
| | QITKHVAQILDSRENTERDENDKVIRDVKVITLKSNLVSQFRKDFELHKVREINDYHHAHDAYLNA |
| | VIGNALLVKYPQLEPEFVYGKFSHFHGHKENKATAKKFFYSNIMALFKKDVEYSDDTGEMVWNK |
| | EKDFATVRKVLSCPQVNIVKKTEVQTGGFSKESILPKRNSDKLIPRKKNWDPKKYGGFDSPTVAYS |
| | VLVIAKVEKGKSKKLKPVKELIGITIMERSSFEKNPIDFLEAKGYKEVRKDLIIKLPKYSLFELENGR |
| | KRMLASAGELQKGNELALPSKYVNFLYLASHYEKLKGSPEDNEQKQLFVEQHKHYLDEIIEQISEF |
| | SERVILADANLDKVLSAYNNHRDKPIREQAENIIHLFTLTNLGAPAAFKFFDTTIDRKRYTSTKEVL |
| | DATLIHQSITGLYETRIDLSQLGGD |
| 239 | MKKSYSIGLDIGTNSVGWSVITDDYKVPAKKMKVLGNTDKKYIKKNLLGALLFDSGETAEATRLK |
| | RTARRRYTRRKNRLRYLQEIFAKEMAKVDESFFQRLEESFLTDDDKNFDSHPIFGNKAEEDAYHDE |
| | FPTIYHLRQYLADSPQKADLRLVYLALAHMIKFRGHFLIEGQLNAENTDVQTLFNDFVEVYDKTVE |
| | ESHLSEITVDALSILTEKVSKSRRLENLVKCYPTEKKNTLFGNLIALSLGLQPNFKTNFKLSEDAKLQ |
| | FSKDTYEEDLEELLGKIGDEYADLFASAKNLYDAILLSGILTVDDNTTKAPLSASMVKRYAEHHED |
| | LEKLKEFVKANKPELYHDIFKDETKNGYAGYIENGVKQDEFYKYLKNTLSKIAGSDYFLDKIERED |
| | FLRKQRTFDNGSIPHQIHLQEMHAILRRQGEYYPFLKENQAKIEKILTFRIPYYVGPLARKNSRFAW |
| | AEYHSDEKITPWNFEEIVDKEESATAFIHRMTNNDLYLPEEKVLPKHSLIYEEFTVYNELTKVKYVT |
| | DQGKAYYFDANMKQEIFDGVFKEHRKVTKDKLMNFLEKEFDEFRIVDLTGLDKENKSFNASLGTY |
| | HDLKKILDKSFLDDKTNETIIEDIIQTLTLFEDRDMIRQRLQKYSDIFTPQQLKKLERRHYTGWGRLS |
| | YKLINGIRNKENGKTILDYLIDDGYANRNFMQLINDDTLPFKQIIQEAQIIGDIDDIETVVHDLPGSPA |
| | IKKGILQSVKIVDELVKVMGHNPDNIVIEMARENQTTNRGRSQSQQRLKKLQESLKELGSNILNEEK |
| | PSYVEDKVENSHLQNDKLFLYYIQNGKDMYTGEELDIDHLSDYDIDHIIPQAFIKDNSIDNRVLTSS |
| | AKNRGKSDDVPSLDIVHDRKADWIRLYKSGLISKRKFDNLTKAEHGGLTEDDKAGFIKRQLVETR |
| | QITKHVAQILDSRENTERDENDKVIRDVKVITLKSNLVSQFRKDFELHKVREINDYHHAHDAYLNA |
| | VIGNALLVKYPQLEPEFVYGKFKHFHGHKENKATAKKFFYSNIMALFKKDVEYSDDTGEMVWNK |
| | EKDFATVRKVLSCPQVNIVKKTEVQTGGFSKESILPKRNSDKLIARKKDWDPKKYGGFDSPTVAYS |
| | VLVVAKVEKGKSKKLKSVKELVGITIMERSSFEKDPVDFLEAKGYKEVRKDLIIKLPKYSLFELENG |
| | RKRMLASAGELQKGNELALPSKYVNFLYLASHYEKLKGSPEDNEQKQLFVEQHKHYLDEIIEQISE |
| | FSKRVILADANLDKVLSAYNKHRDKPIREQAENIIHLFTLTNLGAPAAFKYFDTTIDRKRYTSTKEV |
| | LDSTLIHQSITGLYETRIDLSQLGGD |
| 240 | MKKPYSIGLDIGTNSVGWAVVTDDYKVPAKKMKVLGNTDKSHIKKNLLGALLFDSGNTAEDRRL |
| | KRTARRRYTRRRNRILYLQEIFAEEMNKVDESFFHRLDDSFLVPEDKRGSKYPIFGTLEEEKEYHKQ |
| | FPTIYHLRKQLAESNEKADLRLVYLALAHMIKYRGHFLIDDPKFKVQNNDIQGLFEKFVEEYDNVQ |
| | ETSLSKIKLNVTEILTANIPKSEKQEQLLKNYPSEKKNTLFGNLIGLALGLTPNFKTNFSLENDAKLQI |
| | SSESYEEDLGSLLALIGENFIELFSAVKNLSDGILLAGIVSDESPHAPLSTKMVIRFKEHEEDLAALKH |
| | FIKANLPEKYDEVFSDDSKNGYAGYVGVDSKVRKRNGKLATEEEFYKYLKEILNNLKGADYFLEK |
| | IKREDLLRKQRTFDNGTIPYQVHLTEMKAIIHRQGEHYPFLKENKEKIEKILTFRIPYYVGPLARGNR |
| | DFAWLTRNSDQAIRPWNFEEVVDKASSAEDFINKMTNYDLYLPEEKVLPKHSLLYETFAVYNELT |
| | KVKFIAEGLRDYQFLDSGQKKQIVNQLFKEKRKVTEKDIIHYLHNVDGYDGIELKGIEKQFNASLST |
| | YHDLLKIIKDKEFMDDPKNEEILENIVHTLTIFEDREMIKQRLAQYDSLFDEKVIKALTRRHYTGWG |
| | KLSAKLINGICDKQTGKTILDYLIDDDKINRNFMQLINDDGLSFKEIIQKAQVVGKTDDVKQVVQEL |
| | PGSPAIKKGILQSIKIVDELVKVMGHAPESIVIEMARENQTTARGKKNSQQRYKRIEDALKNLAPGL |
| | DSNILKENPTDNIQLQNDRLFLYYLQNGKDMYTGEALDINQLSNYDIDHIIPQAFIKDDSLDNRVLT |
| | SSKDNRGKSDNVPSLEVVEKMKAFWQQLLDSKLISERKYNNLTKAERERGGLNELDKVGFIKRQL |
| | VETRQITKHVAQILDARFNTEVNENNQKIRKVKIITLKSNLVSNFRKEFGLYKVREINDYHHAHDA |
| | YLNAVVAKAILKKYPKLEPEFVYGDYQKYDIKRYISRSKDPKEIEKATEKYFFYSNLLNFFKEEVH |
| | YADGTIVKRENIEYSKDTGEIAWNKEKDFATVRKVLSCPQVNIVKKTEVQTGGFSKESILPKRNSD |
| | KLIPRKTEWDPKKYGGFDSPTVAYSVLVVAKVEKGKSKKLKSVKELVGITIMERSSFEKDPVDFLE |
| | AKGYKEVRKDLIIKLPKYSLFELENGRRRLLASAGELQKGNELALPSKYVNFLYLASHYEKLKGSP |
| | EDNEQKQLFVEQHKHYLDEIVEQISEFSERVILADANLDKVLSAYNKNRDRPIREQAENIIHLFTLTN |
| | LGAPAAFKFFDTTIDRKRYTSTKEVLDATLIHQSITGLYETRIDLSQLGGD |
| 241 | MKKSYSIGLDIGTNSVGWSVITDDYKVPAKKMKVLGNTDKKYIKKNLLGALLFDSGETAEATRLK |
| | RTARRRYTRRKNRLRYLQEIFAKEMAKVDESFFQRLEESFLTDDDKNFDSHPIFGNKAEEDAYHDE |
| | FPTIYHLRQYLADSPQKADLRLVYLALAHMIKFRGHFLIEGQLNAENTDVQTLFNDFVEVYDKTVE |
| | ESHLSEITVDALSILTEKVSKSRRLENLVKCYPTEKKNTLFGNLIALSLGLQPNFKTNFKLSEDAKLQ |
| | FSKDTYEEDLEELLGKIGDEYADLFASAKNLYDAILLSGILTVDDNTTKAPLSASMVKRYAEHHED |
| | LEKLKEFVKANKPELYHDIFKDETKNGYAGYIENGVKQDEFYKYLKNTLSKIAGSDYFLDKIERED |
| | FLRKQRTFDNGSIPHQIHLQEMHAILRRQGEYYPFLKENQAKIEKILTFRIPYYVGPLARKNSRFAW |
| | AEYHSDEKITPWNFEEIVDKEESATAFIHRMTNNDLYLPEEKVLPKHSLIYEEFTVYNELTKVKYVT |
| | DQGKAYYFDANMKQEIFDGVFKEHRKVTKDKLMNFLEKEFDEFRIVDLTGLDKENKSFNASLGTY |
| | HDLKKILDKSFLDDKTNETIIEDIIQTLTLFEDRDMIRQRLQKYSDIFTPQQLKKLERRHYTGWGRLS |
| | YKLINGIRNKENGKTILDYLIDDGYANRNFMQLINDDTLPFKQIIQEAQIIGDIDDIETVVHDLPGSPA |
| | IKKGILQSVKIVDELVKVMGHNPDNIVIEMARENQTTNRGRSQSQQRLKKLQESLKELGSNILNEEK |
| | PSYVEDKVENSHLQNDKLFLYYIQNGKDMYTGEELDIDHLSDYDIDHIIPQAFIKDNSIDNRVLTSS |
| | AKNRGKSDDVPSLDIVHDRKADWIRLYKSGLISKRKFDNLTKAEHGGLTEDDKAGFIKRQLVETR |
| | QITKHVAQILDSRENTERDENDKVIRDVKVITLKSNLVSQFRKDFELHKVREINDYHHAHDAYLNA |
| | VIGNALLVKYPQLEPEFVYGKFKHFHGHKENKATAKKFFYSNIMALFKKDVEYSDDTGEMVWNK |
| | EKDFATVRKVLSCPQVNIVKKTEVQTGGFSKESILPKRNSDKLIARKKDWDPKKYGGFDSPTVAYS |
| | VLVVAKVEKGKSKKLKSVKELLGITIMERSSFEKNPIDFLEAKGYKEVRKDLIIKLPKYSLFELENG |
| | RKRMLASAGELQKGNELALPSKYVNFLYLASHYEKLKGSPEDNEQKQLFVEQHKHYLDEIIEQISE |
| | FSKRVILADANLDKVLSAYNKHRDKPIREQAENIIHLFTLTNLGAPAAFKYFDTTIDRKRYTSTKEV |
| | LDATLIHQSITGLYETRIDLSQLGGD |
| 242 | MNKPYSIGLDIGTNSVGWSVVDDDYKVPAKKMRVFGDTDKGHIKKNLLGTLLFDSGETAESRRLK |
| | RTARRRYTRRRNRILYLQEIFAKEMAKVDESFFHRLDESFLTDDDKTFDSHPIFGNKAEEDAYHQQ |
| | FPTIYHLRKHLADSTEKADLRLVYLALAHMIKFRGHFLIEGELNAENTDVQKLFADFVGVYDRTFD |
| | DSHLSEITVDASSILTEKISKSRRLEKLIKHYPTEKKNTLFGNLVALALGLQPNFKTNFKLSEDAKLQ |
| | FSKDTYEEDLEELLGKIGDDYADLFTSAKNLYDAILLSGILTVDDESTKAPLSASMIKRYAEHHEDL |
| | EKLKEFVKANKPELYHDIFKDETKNGYAGYIENGVKQDEFYKYLKETLSKQDGSQYFLDKIEREDF |
| | LRKQRTFDNGSIPHQLHLKEMRSILHRQGEYYPFLKENQAKIEKILTFRIPYYVGPLARKDSRFAWA |
| | EYHSDEPITPWNFDEVVDKEKSAEKFITRMTLNDLYLPEEKVLPKHSHVYETYTVYNELTKVKYV |
| | NEQGEAKFFDANMKQEIFDHVFKENRKVTKDKLMNFLGKEFDEFRIVDITGLDKENKAFNASLGT |
| | YHDLKKILDKSFLDDKENETIIEDIIQTLTLFEDRDMIRQRLQKYSDIFTPQQLKKLERRHYTGWGRL |
| | SYKLINGIRDKQTGKTILDYLIDDGYANRNFMQLINDDTLPFKEIIQKAQIIGDIDDIKAVVHDLPGSP |
| | AIKKGILQSVKIVDELVKVMGHNPDNIVIEMARENQTTNRGRTQSQQRLKKLQESLKELGSNILNE |
| | KKPSYVEDKVENTHLQNDRLFLYYIQNGKDMYTGDELDIDHLSDYDIDHIIPQAFIKDDSIDNRVLT |
| | SSAKNRGKSDDVPSIEIVRDRKADWMRLRKAGLISKRKFDNLTKAERGGLTEADKAGFIKRQLVET |
| | RQITKHVAQILDSRENTERDENDKVIREVKVITLKSNLVSQFRKDFQFYKVREINDYHHAHDAYLN |
| | AVVGTALIKKYPKLESEFVYGDYKVYDVRKMIAKSEQEIGKATAKRFFYSNIMNFFKTEIKLANGE |
| | IRKRPLIETNEETGEIVWNKEKDFATVRKVLSCPQVNIVKKTEVQTGGFSKESILPKRNSDKLIPRKK |
| | DWDPKKYGGFDSPTVAYSVLVVAKVEKGKSKKLKSVKELLGITIMERSSFEKNPIDFLEAKGYKEV |
| | KKDLIIKLPKYSLFELENGRKRMLASAGELQKGNELALPSKYVNFLYLASHYEKLKGSPEDNEQKQ |
| | LFVEQHKHYLDEIIEQISEFSERVILADANLDKVLSAYNKHRDKPIREQAENIIHLFTLTNLGAPAAF |
| | KFFDTTIDRKRYTSTKEVLDATLIHQSITGLYETRIDLSQLGGD |
| 243 | MKKSYSIGLDIGTNSVGWSVITDDYKVPAKKMKVLGNTDKKYIKKNLLGALLFDSGETAEATRLK |
| | RTARRRYTRRKNRLRYLQEIFAKEMAKVDESFFQRLEESFLTDDDKNFDSHPIFGNKAEEDAYHDE |
| | FPTIYHLRQYLADSPQKADLRLVYLALAHMIKFRGHFLIEGQLNAENTNVQTLFNDFVEVYDKTVE |
| | ESHLSEITVDALSILTEKVSKSRRLENLVKCYPTEKKNTLFGNLIALSLGLQPNFKTNFKLSEDAKLQ |
| | FSKDTYEEDLEELLGKIGDEYADLFASAKNLYDAILLSGILTVDDNSTKAPLSASMVKRYAEHHED |
| | LEKLKEFVKANKPELYHDIFKDETKNGYAGYIENGVKQDEFYKYLKNTLSKIAGSDYFLDKIERED |
| | FLRKQRTFDNGSIPHQIHLQEMHAILRRQGEHYPFLKENQAKIEKILTFRIPYYVGPLARKNSRFAW |
| | AEYHSDEKITPWNFEEIVDKEESATAFIHRMTNNDLYLPEEKVLPKHSLIYEEFTVYNELTKVKYVT |
| | DQGKAYYFDANMKQEIFDGVFKEHRKVTKDKLMNFLEKEFDEFRIVDLTGLDKENKSFNASLGTY |
| | HDLKKILDKSFLDDKTNETIIEDIIQTLTLFEDRDMIRQRLQKYSDIFTPQQLKKLERRHYTGWGRLS |
| | YKLINGIRNKENGKTILDYLIDDGYANRNFMQLINDDTLPFKQIIKEAQIIGDIDDIETVVHDLPGSPA |
| | IKKGILQSVKIVDELVKVMGHNPDNIVIEMARENQTTNRGRSQSQQRLKKLQESLKELGSNILNEEK |
| | PSYVEDKVENSHLQNDRLFLYYIQNGKDMYTGEELDIDHLSDYDIDHIIPQAFIKDNSIDNRVLTSS |
| | AKNRGKSDDVPSLDIVHDRKADWIRLYKSGLISKRKFDNLTKAEHGGLTEDDKAGFIKRQLVETR |
| | QITKHVAQILDSRENTERDENDKVIRDVKVITLKSNLVSQFRKDFELHKVREINDYHHAHDAYLNA |
| | VIGNALLVKYPQLEPEFVYGKFKHFHGHKENKATAKKFFYSNIMALFKKDVEYSDDTGEMVWNK |
| | EKDFATIRKVLSCPQVNIVKKTEVQTGGFSKESILPKRNSDKLIPRKKNWDPKKYGGFDSPTVAYSV |
| | LVVAKVEKGKSKKLKPVKELIGITIMERNSFEKDPIDFLEAKGYKEVRKDLIIKLPKYSLFELENGRK |
| | RMLASAGELQKGNELALPSKYVNFLYLASHYEKLKGSPEDNEQKQLFVEQHKHYLDEIIEQISEFSE |
| | RVILADANLDKVLSAYNNHRDKPIREQAENIIHLFTLTNLGAPAAFKFFDTTIDRKRYTSTKEILDAT |
| | LIHQSITGLYETRIDLSQLGGD |
| 244 | MKKSYSIGLDIGTNSVGWSVITDDYKVPAKKMKVLGNTDKKYIKKNLLGALLFDSGETAEATRLK |
| | RTARRRYTRRKNRLRYLQEIFAKEMAKVDESFFQRLEESFLTDDDKNFDSHPIFGNKAEEDAYHDE |
| | FPTIYHLRQYLADSPQKADLRLVYLALAHMIKFRGHFLIEGQLNAENTDVQTLFNDFVEVYDKTVE |
| | ESHLSEITVDALSILTEKVSKSRRLENLVKCYPTEKKNTLFGNLIALSLGLQPNFKTNFKLSEDAKLQ |
| | FSKDTYEEDLEELLGKIGDEYADLFASAKNLYDAILLSGILTVDDNTTKAPLSASMVKRYAEHHED |
| | LEKLKEFVKANKPELYHDIFKDETKNGYAGYIENGVKQDEFYKYLKNTLSKIAGSDYFLDKIERED |
| | FLRKQRTFDNGSIPHQIHLQEMHAILRRQGEYYPFLKENQAKIEKILTFRIPYYVGPLARKNSRFAW |
| | AEYHSDEKITPWNFEEIVDKEESATAFIHRMTNNDLYLPEEKVLPKHSLIYEEFTVYNELTKVKYVT |
| | DQGKAYYFDANMKQEIFDGVFKEHRKVTKDKLMNFLEKEFDEFRIVDLTGLDKENKSFNASLGTY |
| | HDLKKILDKSFLDDKTNETIIEDIIQTLTLFEDRDMIRQRLQKYSDIFTPQQLKKLERRHYTGWGRLS |
| | YKLINGIRNKENGKTILDYLIDDGYANRNFMQLINDDTLPFKQIIQEAQIIGDIDDIETVVHDLPGSPA |
| | IKKGILQSVKIVDELVKVMGHNPDNIVIEMARENQTTNRGRSQSQQRLKKLQESLKELGSNILNEEK |
| | PSYVEDKVENSHLQNDKLFLYYIQNGKDMYTGEELDIDHLSDYDIDHIIPQAFIKDNSIDNRVLTSS |
| | AKNRGKSDDVPSLDIVHDRKADWIRLYKSGLISKRKFDNLTKAEHGGLTEDDKAGFIKRQLVETR |
| | QITKHVAQILDSRENTERDENDKVIRDVKVITLKSNLVSQFRKDFELHKVREINDYHHAHDAYLNA |
| | VIGNALLVKYPQLEPEFVYGKFKHFHGHKENKATAKKFFYSNIMALFKKDVEYSDDTGEMVWNK |
| | EKDFATVRKVLSCPQVNIVKKTEVQTGGFSKESILPKRNSDKLIARKKDWDPKKYGGFDSPTVAYS |
| | VLVVAKVEKGKSKKLKSVKELVGITIMERSSFEKDPIDFLEAKGYKEVRKDLIIKLPKYSLFELENG |
| | RKRMLASAGELQKGNELALPSKYVNFLYLASHYEKLKGSPEDNEQKQLFVEQHKHYLDEIIEQISE |
| | FSKRVILADANLDKVLSAYNKHRDKPIREQAENIIHLFTLTNLGAPAAFKYFDTTIDRKRYTSTKEV |
| | LDATLIHQSITGLYETRIDLSQLGGD |
| 245 | MTKPYSIGLDIGTNSVGWAVITDSYKVPSKKMKVLGNTSKKYVKKNLLGALLFESGITAEGRRLK |
| | RTARRRYTRRRNRILYLQEIFSTEMAKIDESFFQRLDDSFLVPDDKRDSKYPIFGNLVKEKTYHDEF |
| | PTIYHLRKYLADSSKKADLRLVYLALAHMIKYRGHFLIEGDFNSRNNDIKKNFQNFLDSYNAIFESD |
| | LSLENSKQLEEIVKDKISKSAKRERVLKLFPDEKSTGLFSEFLKLIVGNQADFKKHFDLEEKAPLQFS |
| | KDTYDEDLENLLGQIGDDFADLFLVAKKLYDAILLSGILTVTDPSTKAPLSASMIERYENHQKDLSA |
| | LKQFIRSNLSEKYDEVFSDQSKDGYAGYINGKTTQEAFYKYIKNLLSKFEGADYFLDKIEREDFLRK |
| | QRTFDNGSIPHQIHLQEMNAIIRRQGEHYPFLKENKEKIEKILTFRIPYYVGPLARGNRDFAWLTRNS |
| | DQAIRPWNFEEVVDKASSAEAFINKMTNFDLYLPEEKVLPKHSLLYETFAVYNELTKVKFIAEGLR |
| | DYQFLDSGQKKQIVNQLFKEKRKVTEKDIIQFLHNVDGYDGIELKGIEKQFNASLATYHDLLKIIKD |
| | KEFMDDPKNEEILENIIHTLTIFEDREMIKQRLAQYDSLFDEKVIKALTRRHYTGWGKLSAKLINGIC |
| | DKQTGKTILDYLIDDGFANRNFMQLINDDTLPFKQIIQKAQIVGDVDDIEQVVHNLPGSPAIKKGIL |
| | QSIKIVDELVKVMGHNPDNIVIEMARENQTTNRGRSQSQQRLKKLQESLKELGSNILNEEKPSYVES |
| | KVENSQLQNDRLFLYYIQNGKDMYTGEELDIDRLSDYDIDHIIPQAFIKDDSIDNRVLTSSAKNRGK |
| | SDDVPSLEVVKKRKTYWHQLLKAKLISQRKFDNLTKAERGGLTEKDKAGFIKRQLVETRQITKHV |
| | AQILDSRFNTKRDENDKVIRDVKVITLKSNLVSQFRKDFGFYKVREINDYHHAHDAYLNAVVGTA |
| | LLKKYPKLAPEFVYGEYKKYDVRKIVRSKDESSLGKATAKMFFYSNLMNFFKKEVRLSDGTVIVR |
| | PVIEYSKDTGEIAWNKEKDFATVRKVLSCPQVNIVKKTEVQTGGFSKESILPKRNSDKLIARKKDW |
| | DPKKYGGFDSPTVAYSVLVVAKVEKGKSKKLKSVKELVGITIMERSSFEKDPVDFLEAKGYKEVR |
| | KDLIIKLPKYSLFELENGRKRMLASAGELQKGNELALPSKYVNFLYLASHYEKLKGSPEDNEQKQL |
| | FVEQHKHYLDEIIEQISEFSKRVILADANLDKVLSAYNKHRDKPIREQAENIIHLFTLTNLGAPAAFK |
| | YFDTTIDRKRYTSTKEVLDSTLIHQSITGLYETRIDLSQLGGD |
| 246 | MKKPYSIGLDIGTNSVGWAVVTDDYKVPAKKMKVLGNTDKSHIKKNLLGALLFDSGNTAEDRRL |
| | KRTARRRYTRRRNRILYLQEIFAEEMNKVDESFFHRLDDSFLVPEDKRGSKYPIFGTLEEEKEYHKQ |
| | FPTIYHLRKQLAESNEKADLRLVYLALAHMIKYRGHFLIDDPKFKVQNNDIQGLFEKFVEEYDNVQ |
| | ETSLSKIKLNVTEILTANIPKSEKQEQLLKNYPSEKKNTLFGNLIGLALGLTPNFKTNFSLENDAKLQI |
| | SSESYEEDLGSLLALIGENFIELFSAVKNLSDGILLAGIVSDESPHAPLSTKMVIRFKEHEEDLAALKH |
| | FIKANLPEKYDEVFSDDSKNGYAGYVGVDSKVRKRNGKLATEEEFYKYLKEILNNLKGADYFLEK |
| | IKREDLLRKQRTFDNGTIPYQVHLTEMKAIIHRQGEHYPFLKENKEKIEKILTFRIPYYVGPLARGNR |
| | DFAWLTRNSDQAIRPWNFEEVVDKASSAEDFINKMTNYDLYLPEEKVLPKHSLLYETFAVYNELT |
| | KVKFIAEGLRDYQFLDSGQKKQIVNQLFKEKRKVTEKDIIHYLHNVDGYDGIELKGIEKQFNASLST |
| | YHDLLKIIKDKEFMDDPKNEEILENIVHTLTIFEDREMIKQRLAQYDSLFDEKVIKALTRRHYTGWG |
| | KLSAKLINGICDKQTGKTILDYLIDDDKINRNFMQLINDDGLSFKEIIQKAQVVGKTDDVKQVVQEL |
| | PGSPAIKKGILQSIKIVDELVKVMGHAPESIVIEMARENQTTARGKKNSQQRYKRIEDALKNLAPGL |
| | DSNILKENPTDNIQLQNDRLFLYYLQNGKDMYTGEALDINQLSNYDIDHIIPQAFIKDDSLDNRVLT |
| | SSKDNRGKSDNVPSLEVVEKMKAFWQQLLDSKLISERKYNNLTKAERERGGLNELDKVGFIKRQL |
| | VETRQITKHVAQILDARFNTEVNENNQKIRKVKIITLKSNLVSNFRKEFGLYKVREINDYHHAHDA |
| | YLNAVVAKAILKKYPKLEPEFVYGDYQKYDIKRYISRSKDPKEIEKATEKYFFYSNLLNFFKEEVH |
| | YADGTIVKRENIEYSKDTGEIAWNKEKDFATVRKVLSCPQVNIVKKTEVQTGGFSKESILPKRNSD |
| | KLIARKKDWDPKKYGGFDSPTVAYSVLVVAKVEKGKSKKLKSVKELVGITIMERSSFEKDPVDFL |
| | EAKGYKEVRKDLIIKLPKYSLFELENGRKRMLASAGELQKGNELALPSKYVNFLYLASHYEKLKGS |
| | PEDNEQKQLFVEQHKHYLDEIIEQISEFSKRVILADANLDKVLSAYNKHRDKPIREQAENIIHLFTLT |
| | NLGAPAAFKYFDTTIDRKRYTSTKEVLDATLIHQSITGLYETRIDLSQLGGD |
| 247 | MKKPYSIGLDIGTNSVGWAVVTDDYKVPAKKMKVLGNTDKSHIKKNLLGALLFEGGETAEGRRL |
| | KRTARRRYTRRRNRILYLQEIFATEMNKVDESFFHRLDDSFLVPEDKRGSKYPIFGTLEEEKEYHKQ |
| | FPTIYHLRKQLAESNEKADLRLVYLALAHMIKYRGHFLIDDPKFKVQNNDIQGLFEKFVEEYDNVQ |
| | ETSLSKIKLNVTEILTANIPKSEKQEQLLKNYPSEKKNTLFGNLIGLALGLTPNFKTNFSLENDAKLQI |
| | SSESYEEDLGNLLALIGENFIELFSAVKNLSDGILLAGIVSDESPHAPLSTKMVIRFKEHEEDLAALK |
| | HFIKANLPEKYDEVFSDDSKNGYAGYVGVDSKVRKRNGKLATEEEFYKYLKDILNNVKGADYFLE |
| | KIKREDFLRKQRTFDNGTIPYQVHLQEMKAILHRQGEHYPFLKENKEKIEKILTFRIPYYVGPLARG |
| | NRDFAWLTRNSDQAIRPWNFEEIVDKASSAEDFINKMTNYDLYLPEEKVLPKHSLLYETFAVYNEL |
| | TKVKFIAEGLRDYQFLDSGQKKQIVNQLFKEKRKVTEKDIIHYLHNVDGYDGIELKGIEKQFNASLS |
| | TYHDLLKIIKDKEFMDDPKNEEILENIVHTLTIFEDREMIKQRLAQYDSLFDEKVIKALTRRHYTGW |
| | GKLSAKLINGIYDKQSQKTILDYLIDDDKINRNFMQLINDDGLSFKEIIQKAQVVGKTDDVKQVVQ |
| | ELPGSPAIKKGILQSIKIVDELVKVMGHAPESIVIEMARENQTTARGKKNSQQRYKRIEDALKNLAP |
| | GLDSNILKENPTDNVQLQNDRLFLYYLQNGRDMYTGKPLDINQLSNYDIDHIIPQAFIKDDSLDNRV |
| | LTSSKDNRGKSDNVPSIEVVEKMKAFWQQLLDSKLISERKYNNLTKAERERGGLNELDKVGFIKR |
| | QLVETRQITKHVAQILDARFNTEVNENNQKIRKVKIITLKSNLVSNFRKEFGLYKVREINDYHHAHD |
| | AYLNAVVAKAILKKYPKLEPEFVYGDYQKYDLKRYISRSKDPKDIEKATEKYFFYSNLLNFFKEEV |
| | HYADGTIIKRENIEYSKDTGEIAWNKEKDFATVRKVLSCPQVNIVKKTEVQTGGFSKESILPKRNSD |
| | KLIARKKDWDPKKYGGFDSPTVAYSVLVVAKVEKGKSKKLKSVKELVGITIMERSSFEKDPVDFL |
| | EAKGYKEVRKDLIIKLPKYSLFELENGRKRMLASAGELQKGNELALPSKYVNFLYLASHYEKLKGS |
| | PEDNEQKQLFVEQHKHYLDEIIEQISEFSKRVILADANLDKVLSAYNKHRDKPIREQAENIIHLFTLT |
| | NLGAPAAFKYFDTTIDRKRYTSTKEVLDATLIHQSITGLYETRIDLSQLGGD |
| 248 | MDKKYSIGLDIGTNSVGWAVITDDYKVPAKKMKVLGNTDKRTIKKNLLGALLFDSGETAEATRLK |
| | RTARRRYTRRKNRLRYLQEIFSKEMSNVDANFFYRLEDSFLVPEDKQQERHPIFGNLVEEIEYHKEF |
| | PTIYHLRKQLADSKEKADLRLIYLALAHMIKYRGHFLFEGDLKSENTDVQRLFKDFVEVYDKTVEG |
| | SYLSENLTNVTDVLVEKISKSRRLENILHYFPNEKKNGLFGNFLALALGLQPNFKNNFDLAEEAKL |
| | QISKDTYEEELEVLLAQIGDNYAELFIATKELYDGILLAGILTVTDSSTKAPLSASMIERYENHQGDL |
| | AKLKAFIRRFCKETYDEVFSDEVKDGYAGYIEGKTSQEDFYVHLKKLLANLEGADYFLEKINREDL |
| | LRKQRTFDNGSIPYQIHLVEMRAILDKQAKFYPFLAKNQDRIEKLLTFRIPYYVGPLARGKSDFAWL |
| | SRKSADKITPWNFDEIVDKESSAEAFINRMTNYDLYLPNQKVLPKHSLLYEKFTVYNELTKVKYKT |
| | EQGKTAFFDANMKQEIFDGVFKVYRKVTKDKLMDFLEKEFDEFRIVDLMGLDKENKAFNASYGT |
| | YHDLRKILDKDFLDNSDNEKILEDIVLTLTLFEDREMIRKRLEKYKDIFTEKQRKQLERRHYTGWG |
| | RLSAKLIHGIRNKESRKTILDYLIDDGYSNRNFMQLINDDTLTFKEEIAKAQVVGETDSLKQFISELA |
| | GSPAIKKGILQTVKIVDELVKVMGHKPENIVIEMARENQTTQKGQKNSQERMKRIEEGIKELGSQIL |
| | KEHPVENTQLQNEKLYLYYLQNGRDMYVDQELDINRLSDYDIDHIIPQAFIKDDSIDNRVLTSSKD |
| | NRGKSDNVPSEDVVKKMKNYWRQLLNAKLISQRKFDNLTKAERGGLSELDKVGFIKRQLVETRQI |
| | TKHVAQILDSRMNTKYDENDKLIRDVKIITLKSKLVSDFRKDFQFYKVREINNYHHAHDAYLNAV |
| | VGTALIKKYPKLESEFVYGDYKVYDVQKMIAKSEQEIGKATAKFFFYSNIMNFFKTEITLANGEIRK |
| | RPMIETNEETGEIVWDKGRDFATVRKVLCMPQVNIVKKTEVQTGGFSKESILPKRNSDKLIARKKD |
| | WDPKKYGGFDSPTVAYSVLVVAKVEKGKSKKLKSVKELVGITIMERSSFEKDPVDFLEAKGYKEV |
| | RKDLIIKLPKYSLFELENGRKRMLASAGELQKGNELALPSKYVNFLYLASHYEKLKGSPEDNEQKQ |
| | LFVEQHKHYLDEIIEQISEFSKRVILADANLDKVLSAYNKHRDKPIREQAENIIHLFTLTNLGAPAAF |
| | KYFDTTIDRKRYTSTKEVLDSTLIHQSITGLYETRIDLSQLGGD |
| 249 | MKKSYSIGLDIGTNSVGWSVITDDYKVPAKKMKVLGNTDKKYIKKNLLGALLFDSGETAEATRLK |
| | RTARRRYTRRKNRLRYLQEIFAKEMAKVDESFFQRLEESFLTDDDKNFDSHPIFGNKAEEDAYHDE |
| | FPTIYHLRQYLADSPQKADLRLVYLALAHMIKFRGHFLIEGQLNAENTDVQTLFNDFVEVYDKTVE |
| | ESHLSEITVDALSILTEKVSKSRRLENLVKCYPTEKKNTLFGNLIALSLGLQPNFKTNFKLSEDAKLQ |
| | FSKDTYEEDLEELLGKIGDEYADLFASAKNLYDAILLSGILTVDDNTTKAPLSASMVKRYAEHHED |
| | LEKLKEFVKANKPELYHDIFKDETKNGYAGYIENGVKQDEFYKYLKNTLSKIAGSDYFLDKIERED |
| | FLRKQRTFDNGSIPHQIHLQEMHAILRRQGEYYPFLKENQAKIEKILTFRIPYYVGPLARKNSRFAW |
| | AEYHSDEKITPWNFEEIVDKEESATAFIHRMTNNDLYLPEEKVLPKHSLIYEEFTVYNELTKVKYVT |
| | DQGKAYYFDANMKQEIFDGVFKEHRKVTKDKLMNFLEKEFDEFRIVDLTGLDKENKSFNASLGTY |
| | HDLKKILDKSFLDDKTNETIIEDIIQTLTLFEDRDMIRQRLQKYSDIFTPQQLKKLERRHYTGWGRLS |
| | YKLINGIRNKENGKTILDYLIDDGYANRNFMQLINDDTLPFKQIIQEAQIIGDIDDIETVVHDLPGSPA |
| | IKKGILQSVKIVDELVKVMGHNPDNIVIEMARENQTTNRGRSQSQQRLKKLQESLKELGSNILNEEK |
| | PSYVEDKVENSHLQNDKLFLYYIQNGKDMYTGEELDIDHLSDYDIDHIIPQAFIKDNSIDNRVLTSS |
| | AKNRGKSDDVPSLDIVHDRKADWIRLYKSGLISKRKFDNLTKAEHGGLTEDDKAGFIKRQLVETR |
| | QITKHVAQILDSRENTERDENDKVIRDVKVITLKSNLVSQFRKDFELHKVREINDYHHAHDAYLNA |
| | VIGNALLVKYPQLEPEFVYGKFKHFHGHKENKATAKKFFYSNIMALFKKDVEYSDDTGEMVWNK |
| | EKDFATVRKVLSCPQVNIVKKTEVQTGGFSKESILPKRNSDKLIARKKDWDPKKYGGFDSPTVAYS |
| | VLVVAKVEKGKSKKLKSVKELLGITIMERSSFEKNPIDFLEAKGYKEVRKDLIIKLPKYSLFELENG |
| | RKRMLASAGELQKGNELALPSKYVNFLYLASHYEKLKGSPEDNEQKQLFVEQHQHYLDEIIEQISE |
| | FSKRVILADANLDKVLSAYNKHRDKPIREQAENIIHLFTLTNLGAPAAFKYFDTTIDRKRYTSTKEV |
| | LDATLIHQSITGLYETRIDLSQLGGD |
| 250 | MTKPYSIGLDIGTNSVGWAVITDSYKVPSKKMKVLGNTSKKYVKKNLLGALLFESGITAEGRRLK |
| | RTARRRYTRRRNRILYLQEIFSTEMAKIDESFFQRLDDSFLVPDDKRDSKYPIFGNLVKEKTYHDEF |
| | PTIYHLRKYLADSSKKADLRLVYLALAHMIKYRGHFLIEGDFNSRNNDIKKNFQNFLDSYNAIFESD |
| | LSLENSKQLEEIVKDKISKSAKRERVLKLFPDEKSTGLFSEFLKLIVGNQADFKKHFDLEEKAPLQFS |
| | KDTYDEDLENLLGQIGDDFADLFLVAKKLYDAILLSGILTVTDPSTKAPLSASMIERYENHQKDLSA |
| | LKQFIRSNLSEKYDEVFSDQSKDGYAGYINGKTTQEAFYKYIKNLLSKFEGADYFLDKIEREDFLRK |
| | QRTFDNGSIPHQIHLQEMNAIIRRQGEHYPFLKENKEKIEKILTFRIPYYVGPLARGNRDFAWLTRNS |
| | DQAIRPWNFEEVVDKASSAEAFINKMTNFDLYLPEEKVLPKHSLLYETFAVYNELTKVKFIAEGLR |
| | DYQFLDSGQKKQIVNQLFKEKRKVTEKDIIQFLHNVDGYDGIELKGIEKQFNASLATYHDLLKIIKD |
| | KEFMDDPKNEEILENIIHTLTIFEDREMIKQRLAQYDSLFDEKVIKALTRRHYTGWGKLSAKLINGIC |
| | DKQTGKTILDYLIDDGFANRNFMQLINDDTLPFKQIIQKAQIVGDVDDIEQVVHNLPGSPAIKKGIL |
| | QSIKIVDELVKVMGHNPDNIVIEMARENQTTNRGRSQSQQRLKKLQESLKELGSNILNEEKPSYVES |
| | KVENSQLQNDRLFLYYIQNGKDMYTGEELDIDRLSDYDIDHIIPQAFIKDDSIDNRVLTSSAKNRGK |
| | SDDVPSLEVVKKRKTYWHQLLKAKLISQRKFDNLTKAERGGLTEKDKAGFIKRQLVETRQITKHV |
| | AQILDSRFNTKRDENDKVIRDVKVITLKSNLVSQFRKDFGFYKVREINDYHHAHDAYLNAVVGTA |
| | LLKKYPKLAPEFVYGEYKKYDVRKIVRSKDESSLGKATAKMFFYSNLMNFFKKEVRLSDGTVIVR |
| | PVIEYSKDTGEIAWNKEKDFATVRKVLSCPQVNIVKKTEVQTGGFSKESILPKRNSDKLIARKKDW |
| | DPKKYGGFDSPTVAYSVLVVAKVEKGKSKKLKSVKELLGITIMERSSFEKNPIDFLEAKGYKEVRK |
| | DLIIKLPKYSLFELENGRKRMLASAGELQKGNELALPSKYVNFLYLASHYEKLKGSPEDNEQKQLF |
| | VEQHKHYLDEIIEQISEFSKRVILADANLDKVLSAYNKHRDKPIREQAENIIHLFTLTNLGAPAAFKY |
| | FDTTIDRKRYTSTKEVLDATLIHQSITGLYETRIDLSQLGGD |
| 251 | MTKPYSIGLDIGTNSVGWAVITDSYKVPSKKMKVLGNTSKKYVKKNLLGALLFESGITAEGRRLK |
| | RTARRRYTRRRNRILYLQEIFSTEMAKIDESFFQRLDDSFLVPDDKRDSKYPIFGNLVKEKTYHDEF |
| | PTIYHLRKYLADSSKKADLRLVYLALAHMIKYRGHFLIEGDFNSRNNDIKKNFQNFLDSYNAIFESD |
| | LSLENSKQLEEIVKDKISKSAKRERVLKLFPDEKSTGLFSEFLKLIVGNQADFKKHFDLEEKAPLQFS |
| | KDTYDEDLENLLGQIGDDFADLFLVAKKLYDAILLSGILTVTDPSTKAPLSASMIERYENHQKDLSA |
| | LKQFIRSNLSEKYDEVFSDQSKDGYAGYINGKTTQEAFYKYIKNLLSKFEGADYFLDKIEREDFLRK |
| | QRTFDNGSIPHQIHLQEMNAIIRRQGEHYPFLKENKEKIEKILTFRIPYYVGPLARGNRDFAWLTRNS |
| | DQAIRPWNFEEVVDKASSAEAFINKMTNFDLYLPEEKVLPKHSLLYETFAVYNELTKVKFIAEGLR |
| | DYQFLDSGQKKQIVNQLFKEKRKVTEKDIIQFLHNVDGYDGIELKGIEKQFNASLATYHDLLKIIKD |
| | KEFMDDPKNEEILENIIHTLTIFEDREMIKQRLAQYDSLFDEKVIKALTRRHYTGWGKLSAKLINGIC |
| | DKQTGKTILDYLIDDGFANRNFMQLINDDTLPFKQIIQKAQIVGDVDDIEQVVHNLPGSPAIKKGIL |
| | QSIKIVDELVKVMGHNPDNIVIEMARENQTTNRGRSQSQQRLKKLQESLKELGSNILNEEKPSYVES |
| | KVENSQLQNDRLFLYYIQNGKDMYTGEELDIDRLSDYDIDHIIPQAFIKDDSIDNRVLTSSAKNRGK |
| | SDDVPSLEVVKKRKTYWHQLLKAKLISQRKFDNLTKAERGGLTEKDKAGFIKRQLVETRQITKHV |
| | AQILDSRFNTKRDENDKVIRDVKVITLKSNLVSQFRKDFGFYKVREINDYHHAHDAYLNAVVGTA |
| | LLKKYPKLAPEFVYGEYKKYDVRKIVRSKDESSLGKATAKMFFYSNLMNFFKKEVRLSDGTVIVR |
| | PVIEYSKDTGEIAWNKEKDFATVRKVLSCPQVNIVKKTEVQTGGFSKESILPKRNSDKLIPRKTEWD |
| | PKKYGGFDSPTVAYSVLVVAKVEKGKSKKLKSVKELVGITIMERSSFEKDPVDFLEAKGYKEVRK |
| | DLIIKLPKYSLFELENGRRRLLASAGELQKGNELALPSKYVNFLYLASHYEKLKGSPEDNEQKQLFV |
| | EQHKHYLDEIVEQISEFSERVILADANLDKVLSAYNKNRDRPIREQAENIIHLFTLTNLGAPAAFKFF |
| | DTTIDRKRYTSTKEVLDATLIHQSITGLYETRIDLSQLGGD |
| 252 | MTKPYSIGLDIGTNSVGWAVITDSYKVPSKKMKVLGNTSKKYVKKNLLGALLFESGITAEGRRLK |
| | RTARRRYTRRRNRILYLQEIFSTEMAKIDESFFQRLDDSFLVPDDKRDSKYPIFGNLVKEKTYHDEF |
| | PTIYHLRKYLADSSKKADLRLVYLALAHMIKYRGHFLIEGDFNSRNNDIKKNFQNFLDSYNAIFESD |
| | LSLENSKQLEEIVKDKISKSAKRERVLKLFPDEKSTGLFSEFLKLIVGNQADFKKHFDLEEKAPLQFS |
| | KDTYDEDLENLLGQIGDDFADLFLVAKKLYDAILLSGILTVTDPSTKAPLSASMIERYENHQKDLSA |
| | LKQFIRSNLSEKYDEVFSDQSKDGYAGYINGKTTQEAFYKYIKNLLSKFEGADYFLDKIEREDFLRK |
| | QRTFDNGSIPHQIHLQEMNAIIRRQGEHYPFLKENKEKIEKILTFRIPYYVGPLARGNRDFAWLTRNS |
| | DQAIRPWNFEEVVDKASSAEAFINKMTNFDLYLPEEKVLPKHSLLYETFAVYNELTKVKFIAEGLR |
| | DYQFLDSGQKKQIVNQLFKEKRKVTEKDIIQFLHNVDGYDGIELKGIEKQFNASLATYHDLLKIIKD |
| | KEFMDDPKNEEILENIIHTLTIFEDREMIKQRLAQYDSLFDEKVIKALTRRHYTGWGKLSAKLINGIC |
| | DKQTGKTILDYLIDDGFANRNFMQLINDDTLPFKQIIQKAQIVGDVDDIEQVVHNLPGSPAIKKGIL |
| | QSIKIVDELVKVMGHNPDNIVIEMARENQTTNRGRSQSQQRLKKLQESLKELGSNILNEEKPSYVES |
| | KVENSQLQNDRLFLYYIQNGKDMYTGEELDIDRLSDYDIDHIIPQAFIKDDSIDNRVLTSSAKNRGK |
| | SDDVPSLEVVKKRKTYWHQLLKAKLISQRKFDNLTKAERGGLTEKDKAGFIKRQLVETRQITKHV |
| | AQILDSRFNTKRDENDKVIRDVKVITLKSNLVSQFRKDFGFYKVREINDYHHAHDAYLNAVVGTA |
| | LLKKYPKLAPEFVYGEYKKYDVRKIVRSKDESSLGKATAKMFFYSNLMNFFKKEVRLSDGTVIVR |
| | PVIEYSKDTGEIAWNKEKDFATVRKVLSCPQVNIVKKTEVQTGGFSKESILPKRNSDKLIARKKDW |
| | DPKKYGGFDSPTVAYSVLVVAKVEKGKSKKLKSVKELVGITIMERSSFEKDPVDFLEAKGYKEVR |
| | KDLIIKLPKYSLFELENGRKRMLASAGELQKGNELALPSKYVNFLYLASHYEKLKGSPEDNEQKQL |
| | FVEQHQHYLDEIIEQISEFSKRVILADANLDKVLSAYNKHRDKPIREQAENIIHLFTLTNLGAPAAFK |
| | YFDTTIDRKRYTSTKEVLDATLIHQSITGLYETRIDLSQLGGD |
| 253 | MKKPYSIGLDIGTNSVGWAIVDDDYKVPAKKMRVFGDTDRSHIKKNLLGTLLFDSGETAESRRLK |
| | RTARRRYTRRRNRILYLQEIFAEEMNKVDDSFFHRLEDSFLVEEDKQGSKYPIFGTLEEEKEYHKQF |
| | PTIYHLRKQLAESNEKADLRLIYLALAHMIKYRGHFLIDDPKFKVQNNDIQGLFEKFVEEYDNVQE |
| | TSLSKIKLNVTEILTANIPKSEKQEQLLKNYPSEKKNTLFGNLIGLALGLTPNFKTNFSLENDAKLQIS |
| | SESYEEDLGNLLALIGENFIELFSAVKNLSDGILLAGIVSDESPHAPLSTKMVIRFKEHEEDLAALKH |
| | FIKANLPEKYDEVFSDDSKNGYAGYVGVDSKVRKRNGKLATEEEFYKYLKDILNNVKGADYFLEK |
| | IKREDFLRKQRTFDNGSIPYQVHLTEMKAILQRQGEHYPFLKENKEKIEKILTFRIPYYVGPLARGNR |
| | DFAWLTRNSDQAIRPWNFEEVVDKASSAEDFINKMTNYDLYLPEEKVLPKHSLLYETFAVYNELT |
| | KVKFIAEGLRDYQFLDSGQKKQIVNQLFKEKRKVTEKDIIHYLHNVDGYDGIELKGIEKQFNASLST |
| | YHDLLKIIKDKEFMDDPKNEEILENIVHTLTIFEDREMIKQRLAQYDSIFDEKVIKALTRRHYTGWG |
| | KLSAKLINGICDKKTGKTILDYLIDDGKINRNFMQLINDDGLSFKEIIQKAQVVGKTDDVKQVVQEL |
| | PGSPAIKKGILQSIKIVDELVKVMGHAPESIVIEMARENQTTARGKKNSQQRYKRIEDALKNLAPGL |
| | DSNILKENPTDNIQLQNDRLFLYYLQNGKDMYTGEPLDINQLSSYDIDHIIPQAFIKDDSLDNRVLTS |
| | SKDNRGKSDNVPSIEVVQKRKAFWQQLLDSKLISERKFNNLTKAERGGLDERDKVGFIKRQLVETR |
| | QITKHVAQILDARFNTEVDENNQKIREVKIITLKSNLVSNFRKEFGLYKVREINDYHHAHDAYLNA |
| | VVAKAILKKYPKLEPEFVYGDYQKYDIKRYISRSKDPKDIEKATEKYFFYSNLLNFFKEEVHYADG |
| | TIIKRENIEYSKDTGEIAWNKEKDFATVRKVLSCPQVNIVKKTEVQTGGFSKESILPKRNSDKLIPRK |
| | TEWDPKKYGGFDSPTVAYSVLVVAKVEKGKSKKLKSVKELVGITIMERSSFEKDPVDFLEAKGYK |
| | EVRKDLIIKLPKYSLFELENGRRRLLASAGELQKGNELALPSKYVNFLYLASHYEKLKGSPEDNEQ |
| | KQLFVEQHKHYLDEIVEQISEFSERVILADANLDKVLSAYNKNRDRPIREQAENIIHLFTLTNLGAPA |
| | AFKFFDTTIDRKRYTSTKEVLDATLIHQSITGLYETRIDLSQLGGD |
| 254 | MTKPYSIGLDIGTNSVGWAVITDSYKVPSKKMKVLGNTSKKYVKKNLLGALLFESGITAEGRRLK |
| | RTARRRYTRRRNRILYLQEIFSTEMAKIDESFFQRLDDSFLVPDDKRDSKYPIFGNLVKEKTYHDEF |
| | PTIYHLRKYLADSSKKADLRLVYLALAHMIKYRGHFLIEGDFNSRNNDIKKNFQNFLDSYNAIFESD |
| | LSLENSKQLEEIVKDKISKSAKRERVLKLFPDEKSTGLFSEFLKLIVGNQADFKKHFDLEEKAPLQFS |
| | KDTYDEDLENLLGQIGDDFADLFLVAKKLYDAILLSGILTVTDPSTKAPLSASMIERYENHQKDLSA |
| | LKQFIRSNLSEKYDEVFSDQSKDGYAGYINGKTTQEAFYKYIKNLLSKFEGADYFLDKIEREDFLRK |
| | QRTFDNGSIPHQIHLQEMNAIIRRQGEHYPFLKENKEKIEKILTFRIPYYVGPLARGNRDFAWLTRNS |
| | DQAIRPWNFEEVVDKASSAEAFINKMTNFDLYLPEEKVLPKHSLLYETFAVYNELTKVKFIAEGLR |
| | DYQFLDSGQKKQIVNQLFKEKRKVTEKDIIQFLHNVDGYDGIELKGIEKQFNASLATYHDLLKIIKD |
| | KEFMDDPKNEEILENIIHTLTIFEDREMIKQRLAQYDSLFDEKVIKALTRRHYTGWGKLSAKLINGIC |
| | DKQTGKTILDYLIDDGFANRNFMQLINDDTLPFKQIIQKAQIVGDVDDIEQVVHNLPGSPAIKKGIL |
| | QSIKIVDELVKVMGHNPDNIVIEMARENQTTNRGRSQSQQRLKKLQESLKELGSNILNEEKPSYVES |
| | KVENSQLQNDRLFLYYIQNGKDMYTGEELDIDRLSDYDIDHIIPQAFIKDDSIDNRVLTSSAKNRGK |
| | SDDVPSLEVVKKRKTYWHQLLKAKLISQRKFDNLTKAERGGLTEKDKAGFIKRQLVETRQITKHV |
| | AQILDSRFNTKRDENDKVIRDVKVITLKSNLVSQFRKDFGFYKVREINDYHHAHDAYLNAVVGTA |
| | LLKKYPKLAPEFVYGEYKKYDVRKIVRSKDESSLGKATAKMFFYSNLMNFFKKEVRLSDGTVIVR |
| | PVIEYSKDTGEIAWNKEKDFATVRKVLSCPQVNIVKKTEVQTGGFSKESILPKRNSDKLIARKKDW |
| | DPKKYGGFDSPTVAYSVLVVAKVEKGKSKKLKSVKELVGITIMERSSFEKDPVDFLEAKGYKEVR |
| | KDLIIKLPKYSLFELENGRKRMLASAGELQKGNELALPSKYVNFLYLASHYEKLKGSPEDNEQKQL |
| | FVEQHKHYLDEIIEQISEFSKRVILADANLDKVLSAYNKHRDKPIREQAENIIHLFTLTNLGAPAAFK |
| | YFDTTIDRKRYTSTKEVLDATLIHQSITGLYETRIDLSQLGGD |
| 255 | MTKPYSIGLDIGTNSVGWAVITDSYKVPSKKMKVLGNTSKKYVKKNLLGALLFESGITAEGRRLK |
| | RTARRRYTRRRNRILYLQEIFSTEMAKIDESFFQRLDDSFLVPDDKRDSKYPIFGNLVKEKTYHDEF |
| | PTIYHLRKYLADSSKKADLRLVYLALAHMIKYRGHFLIEGDFNSRNNDIKKNFQNFLDSYNAIFESD |
| | LSLENSKQLEEIVKDKISKSAKRERVLKLFPDEKSTGLFSEFLKLIVGNQADFKKHFDLEEKAPLQFS |
| | KDTYDEDLENLLGQIGDDFADLFLVAKKLYDAILLSGILTVTDPSTKAPLSASMIERYENHQKDLSA |
| | LKQFIRSNLSEKYDEVFSDQSKDGYAGYINGKTTQEAFYKYIKNLLSKFEGADYFLDKIEREDFLRK |
| | QRTFDNGSIPHQIHLQEMNAIIRRQGEHYPFLKENKEKIEKILTFRIPYYVGPLARGNRDFAWLTRNS |
| | DQAIRPWNFEEVVDKASSAEAFINKMTNFDLYLPEEKVLPKHSLLYETFAVYNELTKVKFIAEGLR |
| | DYQFLDSGQKKQIVNQLFKEKRKVTEKDIIQFLHNVDGYDGIELKGIEKQFNASLATYHDLLKIIKD |
| | KEFMDDPKNEEILENIIHTLTIFEDREMIKQRLAQYDSLFDEKVIKALTRRHYTGWGKLSAKLINGIC |
| | DKQTGKTILDYLIDDGFANRNFMQLINDDTLPFKQIIQKAQIVGDVDDIEQVVHNLPGSPAIKKGIL |
| | QSIKIVDELVKVMGHNPDNIVIEMARENQTTNRGRSQSQQRLKKLQESLKELGSNILNEEKPSYVES |
| | KVENSQLQNDRLFLYYIQNGKDMYTGEELDIDRLSDYDIDHIIPQAFIKDDSIDNRVLTSSAKNRGK |
| | SDDVPSLEVVKKRKTYWHQLLKAKLISQRKFDNLTKAERGGLTEKDKAGFIKRQLVETRQITKHV |
| | AQILDSRFNTKRDENDKVIRDVKVITLKSNLVSQFRKDFGFYKVREINDYHHAHDAYLNAVVGTA |
| | LLKKYPKLAPEFVYGEYKKYDVRKIVRSKDESSLGKATAKMFFYSNLMNFFKKEVRLSDGTVIVR |
| | PVIEYSKDTGEIAWNKEKDFATVRKVLSCPQVNIVKKTEVQTGGFSKESILPKRNSDKLIARKKDW |
| | DPKKYGGFDSPTVAYSVLVVAKVEKGKSKKLKSVKELVGITIMERSSFEKDPIDFLEAKGYKEVRK |
| | DLIIKLPKYSLFELENGRKRMLASAGELQKGNELALPSKYVNFLYLASHYEKLKGSPEDNEQKQLF |
| | VEQHKHYLDEIIEQISEFSKRVILADANLDKVLSAYNKHRDKPIREQAENIIHLFTLTNLGAPAAFKY |
| | FDTTIDRKRYTSTKEVLDATLIHQSITGLYETRIDLSQLGGD |
| 256 | MTKPYSIGLDIGTNSVGWAVITDSYKVPSKKMKVLGNTSKKYVKKNLLGALLFESGITAEGRRLK |
| | RTARRRYTRRRNRILYLQEIFSTEMAKIDESFFQRLDDSFLVPDDKRDSKYPIFGNLVKEKTYHDEF |
| | PTIYHLRKYLADSSKKADLRLVYLALAHMIKYRGHFLIEGDFNSRNNDIKKNFQNFLDSYNAIFESD |
| | LSLENSKQLEEIVKDKISKSAKRERVLKLFPDEKSTGLFSEFLKLIVGNQADFKKHFDLEEKAPLQFS |
| | KDTYDEDLENLLGQIGDDFADLFLVAKKLYDAILLSGILTVTDPSTKAPLSASMIERYENHQKDLSA |
| | LKQFIRSNLSEKYDEVFSDQSKDGYAGYINGKTTQEAFYKYIKNLLSKFEGADYFLDKIEREDFLRK |
| | QRTFDNGSIPHQIHLQEMNAIIRRQGEHYPFLKENKEKIEKILTFRIPYYVGPLARGNRDFAWLTRNS |
| | DQAIRPWNFEEVVDKASSAEAFINKMTNFDLYLPEEKVLPKHSLLYETFAVYNELTKVKFIAEGLR |
| | DYQFLDSGQKKQIVNQLFKEKRKVTEKDIIQFLHNVDGYDGIELKGIEKQFNASLATYHDLLKIIKD |
| | KEFMDDPKNEEILENIIHTLTIFEDREMIKQRLAQYDSLFDEKVIKALTRRHYTGWGKLSAKLINGIC |
| | DKQTGKTILDYLIDDGFANRNFMQLINDDTLPFKQIIQKAQIVGDVDDIEQVVHNLPGSPAIKKGIL |
| | QSIKIVDELVKVMGHNPDNIVIEMARENQTTNRGRSQSQQRLKKLQESLKELGSNILNEEKPSYVES |
| | KVENSQLQNDRLFLYYIQNGKDMYTGEELDIDRLSDYDIDHIIPQAFIKDDSIDNRVLTSSAKNRGK |
| | SDDVPSLEVVKKRKTYWHQLLKAKLISQRKFDNLTKAERGGLTEKDKAGFIKRQLVETRQITKHV |
| | AQILDSRFNTKRDENDKVIRDVKVITLKSNLVSQFRKDFGFYKVREINDYHHAHDAYLNAVVGTA |
| | LLKKYPKLAPEFVYGEYKKYDVRKIVRSKDESSLGKATAKMFFYSNLMNFFKKEVRLSDGTVIVR |
| | PVIEYSKDTGEIAWNKEKDFATVRKVLSCPQVNIVKKTEVQTGGFSKESILPKRNSDKLIPRKTEWD |
| | PKKYGGFDSPIVAYSVLVVAKVEKGKSKKLKPVKELVGITIMERSSFEKDPVDFLEAKGYKEVRKD |
| | LIIKLPKYSLFELENGRKRMLASAGELQKGNELALPSKYVNFLYLASHYEKLKGSPEDNEQKQLFV |
| | EQHKHYLDEIVEQISEFSERVILADANLDKILSAYNKHRDKPIREQAENIINLFTFTNLGAPAAFKFF |
| | DTDIDRKRYTSTKEVLDATLIHQSITGLYETRIDLSQLGGD |
| 257 | MKKPYSIGLDIGTNSVGWAVVTDDYKVPAKKMKVLGNTDKSHIKKNLLGALLFDSGETAEGRRL |
| | KRTARRRYTRRRNRILYLQEIFAEEMNKIDESFFHRLDDSFLVPEDKRGSKYPIFATLEEEKEYHKK |
| | FPTIYHLRKHLAESNEKADLRLVYLALAHMIKYRGHFLIDDPKFKVQNNDIQGLFEKFVEEYDNVQ |
| | ETSLSKIKLNVTEILTANIPKSEKQEQLLKNYPSEKKNTLFGNLIGLALGLTPNFKTNFSLENDAKLQI |
| | SSESYEEDLGNLLALIGENFIELFSAVKNLSDGILLAGIVSDESPHAPLSTKMVIRFKEHEEDLAALK |
| | HFIKANLPEKYDEVFSDDSKNGYAGYVGVDSKVRKRNGKLATEEEFYKYLKDILNNVKGADYFLE |
| | KIKREDLLRKQRTFDNGTIPYQVHLSEMKAIIHRQGEHYPFLKENKEKIEKILTFRIPYYVGPLARGN |
| | RDFAWLTRNSDQAIRPWNFEEIVDKASSAEDFINKMTNYDLYLPEEKVLPKHSLLYETFAVYNELT |
| | KVKFIAEGLRDYQFLDSGQKKQIVNQLFKEKRKVTEKDIIHYLHNVDGYDGIELKGIEKQFNASLST |
| | YHDLLKIIKDKEFMDDPKNEEILENIVHTLTIFEDREMIKQRLAQYDSLFDEKVIKALTRRHYTGWG |
| | KLSAKLINGIYDKQSKKTILDYLIDDGEINRNFMQLIHDDGLSFKEIIQKAQVFGKTDDVKQVVQEL |
| | PGSPAIKKGILQSIKIVDELVKVMGHAPESIVIEMARENQTTARGKKNSQQRYKRIEDSLKNLASGL |
| | DSNILKENPTDNVQLQNDRLFLYYLQNGKDMYTGEALDINQLSNYDIDHIIPQAFIKDDSLDNRVLT |
| | SSKDNRGKSDNVPSIEVVEKMKAFWQQLLDSKLISERKYNNLTKAERERGGLNELDKVGFIKRQL |
| | VETRQITKHVAQILDARFNTEVNENNQKIRKVKIITLKSNLVSNFRKEFGLYKVREINDYHHAHDA |
| | YLNAVVAKAILKKYPKLEPEFVYGDYQKYDIKRYISRSKDPKDIEKATEKYFFYSNLLNFFKEEVH |
| | YADGTIIKRENIEYSKDTGEIAWNKEKDFATVRKVLSCPQVNIVKKTEVQTGGFSKESILPKRNSDK |
| | LIPRKTEWDPKKYGGFDSPTVAYSVLVVAKVEKGKSKKLKSVKELVGITIMERSSFEKDPVDFLEA |
| | KGYKEVRKDLIIKLPKYSLFELENGRRRLLASAGELQKGNELALPSKYVNFLYLASHYEKLKGSPE |
| | DNEQKQLFVEQHKHYLDEIVEQISEFSERVILADANLDKVLSAYNKNRDRPIREQAENIIHLFTLTNL |
| | GAPAAFKFFDTTIDRKRYTSTKEVLDATLIHQSITGLYETRIDLSQLGGD |
| 258 | MKKPYSIGLDIGTNSVGWAVITDDYKVPAKKMKVLGNTDKSHIKKNLLGALLFDSGNTAEDRRLK |
| | RTARRRYTRRRNRILYLQEIFAEEMNKIDESFFHRLDDSFLVPEDKRGSKYPIFATLEEEKEYHKQFP |
| | TIYHLRKKLAESNEKADLRLIYLALAHMIKYRGHFLIDDPKFKVQNNDIQGLFEKFVEEYDNVQET |
| | SLSKIKLNVTEILTANIPKSEKQEQLLKNYPSEKKNTLFGNLIGLALGLTPNFKTNFSLENDAKLQISS |
| | ESYEEDLGNLLALIGENFIELFSAVKNLSDGILLAGIVSDESPHAPLSTKMVIRFKEHEEDLAALKHFI |
| | KANLPEKYDEVFSDDSKNGYAGYVGVDSKVRKRNGKLATEEEFYKYLKEILNNVKGADYFLEKI |
| | KREDFLRKQRTFDNGTIPYQVHLAEMRAILHRQGDYYPFLKDNKEKIEKILTFRIPYYVGPLARGNR |
| | DFAWLTRNSDQAIRPWNFEEVVDKASSAEDFINKMTNYDLYLPEEKVLPKHSLLYETFAVYNELT |
| | KVKFIAEGLRDYQFLDSGQKKQIVNQLFKEKRKVTEKDIIQFLHNVDGYDGIELKGIEKQFNASLST |
| | YHDLLKIIKDKEFMDDPKNEEILENIVHTLTIFEDREMIKQRLAQYDSLFDEKVIKALTRRHYTGWG |
| | KLSAKLINGIRDKQSNKTILDYLIDDDKINRNFMQLINDDGLSFKDIIQKAQVVGKTDDVKQVVQEL |
| | PGSPAIKKGILQSIKIVDELVKVMGHAPESIVIEMARENQTTARGKKNSQQRYKRIEDALKNLAPGL |
| | DSNILKENPTDNIQLQNDRLFLYYLQNGKDMYTGESLDINQLSNYDIDHIVPQAFIKDDSLDNRVLT |
| | SSKDNRGKSDNVPSLEVVQKRKAFWQQLLDSKLISERKENNLTKAERGGLDERDKVGFIKRQLVE |
| | TRQITKHVAQILDARFNTEVNEKNQKIRKVKIITLKSNLVSNFRKEFRLYKVREINDYHHAHDAYL |
| | NAVVAKAILKKYPKLEPEFVYGDYQKYDIKRYISKSKDPKDIEKATQKYFFYSNLLNFFKEEVHYA |
| | DGTIVKRENVEYSKDTGEIAWNKEKDFATVRKVLSCPQVNIVKKTEVQTGGFSKESILPKRNSDKLI |
| | PRKTEWDPKKYGGFDSPTVAYSVLVVAKVEKGKSKKLKSVKELVGITIMERSSFEKDPVDFLEAK |
| | GYKEVRKDLIIKLPKYSLFELENGRRRLLASAGELQKGNELALPSKYVNFLYLASHYEKLKGSPED |
| | NEQKQLFVEQHKHYLDEIVEQISEFSERVILADANLDKVLSAYNKNRDRPIREQAENIIHLFTLTNLG |
| | APAAFKFFDTTIDRKRYTSTKEVLDATLIHQSITGLYETRIDLSQLGGD |
| 259 | MDKKYSIGLDIGTNSVGWAVITDDYKVPAKKMKVLGNTDKRTIKKNLLGALLFDSGETAEATRLK |
| | RTARRRYTRRKNRLRYLQEIFSKEMSNVDANFFYRLEDSFLVPEDKQQERHPIFGNLVEEIEYHKEF |
| | PTIYHLRKQLADSKEKADLRLIYLALAHMIKYRGHELFEGDLKSENTDVQRLFKDFVEVYDKTVEG |
| | SYLSENLTNVTDVLVEKISKSRRLENILHYFPNEKKNGLFGNFLALALGLQPNFKNNFDLAEEAKL |
| | QISKDTYEEELEVLLAQIGDNYAELFIATKELYDGILLAGILTVTDSSTKAPLSASMIERYENHQGDL |
| | AKLKAFIRRFCKETYDEVFSDEVKDGYAGYIEGKTSQEDFYVHLKKLLANLEGADYFLEKINREDL |
| | LRKQRTFDNGSIPYQIHLVEMRAILDKQAKFYPFLAKNQDRIEKLLTFRIPYYVGPLARGKSDFAWL |
| | SRKSADKITPWNFDEIVDKESSAEAFINRMTNYDLYLPNQKVLPKHSLLYEKFTVYNELTKVKYKT |
| | EQGKTAFFDANMKQEIFDGVFKVYRKVTKDKLMDFLEKEFDEFRIVDLMGLDKENKAFNASYGT |
| | YHDLRKILDKDFLDNSDNEKILEDIVLTLTLFEDREMIRKRLEKYKDIFTEKQRKQLERRHYTGWG |
| | RLSAKLIHGIRNKESRKTILDYLIDDGYSNRNFMQLINDDTLTFKEEIAKAQVVGETDSLKQFISELA |
| | GSPAIKKGILQTVKIVDELVKVMGHKPENIVIEMARENQTTQKGQKNSQERMKRIEEGIKELGSQIL |
| | KEHPVENTQLQNEKLYLYYLQNGRDMYVDQELDINRLSDYDIDHIIPQAFIKDDSIDNRVLTSSKD |
| | NRGKSDNVPSEDVVKKMKNYWRQLLNAKLISQRKFDNLTKAERGGLSELDKVGFIKRQLVETRQI |
| | TKHVAQILDSRMNTKYDENDKLIRDVKIITLKSKLVSDFRKDFQFYKVREINNYHHAHDAYLNAV |
| | VGTALIKKYPKLESEFVYGDYKVYDVQKMIAKSEQEIGKATAKFFFYSNIMNFFKTEITLANGEIRK |
| | RPMIETNEETGEIVWDKGRDFATVRKVLCMPQVNIVKKTEVQTGGFSKESILPKRNSDKLIARKKD |
| | WDPKKYGGFDSPTVAYSVLVVAKVEKGKSKKLKSVKELVGITIMERSSFEKDPVDFLEAKGYKEV |
| | RKDLIIKLPKYSLFELENGRKRMLASAGELQKGNELALPSKYVNFLYLASHYEKLKGSPEDNEQKQ |
| | LFVEQHQHYLDEIIEQISEFSKRVILADANLDKVLSAYNKHRDKPIREQAENIIHLFTLTNLGAPAAF |
| | KYFDTTIDRKRYTSTKEVLDSTLIHQSITGLYETRIDLSQLGGD |
| 260 | MKKPYSIGLDIGTNSVGWAVVTDDYKVPAKKMKVLGDTDKSHIKKNLLGTLLFESGETAEGRRLK |
| | RTARRRYTRRRNRILYLQEIFAEEMNKVDDSFFQRLDESFLKDDDKNFDSHPIFGNKAEEDAYHQK |
| | FPTIYHLRQYLADSPEKADLRLVYLALAHIIKYRGHELFEGDLKSENKDVQHLFNDFVEMFDKTVE |
| | GSYLSENLPNVADVLVEKVSKSRRLENVLHYFPNEKKNGLFGNFIALALGLQPNFKTNFKLSEDAK |
| | LQFSKDTYDEDLEELLGKIGDDYADLFVAAKNLYDAILLSGILTVTDPTTKAPLSASMIDRYEKHQ |
| | KDLALLKNLIKKNLTQEKYDEVFSDESKNGYAGYINGKVKQEEFYKYLKNTLSKIEGSDYFLDKIE |
| | REDFLRKQRTFDNGSIPHQIHLQEMNAIIRRQGEHYPFLKENKEKIEKILTFRIPYYVGPLARGNRDF |
| | AWLTRNSDQAIRPWNFEEIVDQASSAEDFINKMTNYDLYLPEEKVLPKHSLLYETFAVYNELTKVK |
| | FIAEGLRDYQFLDSGQKKQIVNQLFKEKRKVTEKDIIQYLHNVDGYDGIELKGIEKQFNASLSTYHD |
| | LLKIIKDKEFMDDPKNEEILENIVHTLTIFEDREMIKQRLAQYDSLFDEKVIKALTRRHYTGWGKLS |
| | AKLINGIYDKQSKKTILDYLIDDGEINRNFMQLINDDGLSFKDIIQKAQVVGKTNDVKQVVQELPGS |
| | PAIKKGILQSIKLVDELVKVMGHAPESIVIEMARENQTTARGKKNSQQRYKRIEDALKNLAPGLDS |
| | NILKENPTDNIQLQNDRLFLYYLQNGKDMYTGEALDINQLSNYDIDHIVPQAFIKDDSLDNRVLTSS |
| | KDNRGKSDNVPSIEVVQKRKAFWQQLLDSKLISERKFNNLTKAERGGLDERDKVGFIKRQLVETR |
| | QITKHVAQILDARFNTEVDENNQRIRKVKIITLKSNLVSNFRKEFGLYKVREINDYHHAHDAYLNA |
| | VVAKAILKKYPKLEPEFVYGDYQKYDLKRYISRSKDPKDIEKATEKYFFYSNLLNFFKEEVHYADG |
| | TIIKRENIEYSNDTGEIAWNKEKDFATVRKVLSCPQVNIVKKTEVQTGGFSKESILPKRNSDKLIPRK |
| | KDWDPKKYGGFDSPTVAYSVLVVAKVEKGKSKKLKPVKELVGITIMERSSFEKDPVDFLEAKGYK |
| | EVRKDLIIKLPKYSLFELENGRKRMLASAGELQKGNELALPSKYVNFLYLASHYEKLKGSPEDNEQ |
| | KQLFVEQHKHYLDEIIEQISEFSERVILADANLDKVLSAYNNHRDKPIREQAENIIHLFTLTNLGAPA |
| | AFKFFDTTIDRKRYTSTKEVLDATLIHQSITGLYETRIDLSKLGGD |
| 261 | MKKPYSIGLDIGTNSVGWAVVTDDYKVPAKKMKVLGNTNKESIKKNLIGALLFDAGNTAADRRL |
| | KRTARRRYTRRRNRILYLQEIFAEEMNKVDESFFHRLDDSFLVPEDKRGSKYPIFATLEEEKEYHKK |
| | FPTIYHLRKHLADSKEKADLRLIYLALAHMIKYRGHFLYEESFDIKNNDIQKIFNEFISIYDNTFEGSS |
| | LSGQNAQVEAIFTDKISKSAKRERVLKLFPDEKSTGLFSEFLKLIVGNQADFKKHFDLEEKAPLQFS |
| | KDTYDEDLENLLGQIGDDFADLFLVAKKLYDAILLSGILTVTDPSTKAPLSASMIERYENHQKDLA |
| | ALKQFIQNNLQEKYDEVFSDQSKDGYAGYIDGKTTQEAFYKYIKNLLSKLEGADYFLDKIEREDFL |
| | RKQRTFDNGSIPHQIHLQEMNAIIRRQGEHYPFLKENKEKIEKILTFRIPYYVGPLARGNRDFAWLTR |
| | NSDQAIRPWNFEEIVDQASSAEDFINKMTNYDLYLPEEKVLPKHSLLYETFAVYNELTKVKFIAEG |
| | MRDYQFLDSGQKKKIVSQLFEDKRKVTEKDIIHYLHTIDGYDGIELSGIEKQFNASLSTYHDLLKIIK |
| | DKAFLDDSENEAIIEEIIHTLTIFEDREMIKQRLAQYADIFDKSVLKKLARRHYTGWGKLSAKLINGI |
| | RDKQSNKTILDYLIDDGKINRNFMQLINDDSLSFKETIQKAQVVGDTDDVKQVVQELPGSPAIKKGI |
| | LQSIKIVDELVKVMGHNPDNIVIEMARENQTTNRGRSQSQQRLKKLQDSLKELGSNILNEEKPSYIE |
| | DKVENSHLQNDQLFLYYIQNGKDMYTGEELDIDHLSDYDIDHIIPQAFIKDDSIDNRVLTSSAKNRG |
| | KSDDVPSLDIVRERKADWIRLYKSKLISKRKFDNLTKAERGGLTEADKAGFIKRQLVETRQITKHV |
| | AQILDSRFNTKRDENDKVIRDVKVITLKSNLVSQFRKDFELYKVREINDYHHAHDAYLNAVVGTA |
| | LLKKYPKLAPEFVYGEYKKYDIRKFITSSKDKATAKYFFYSNLMNFFKRIIRYSNGKIVVRPVVEYS |
| | KDTGEIAWNKEKDFATVRKVLSCPQVNIVKKTEVQTGGFSKESILPKRNSDKLIARKKDWDPKKY |
| | GGFDSPTVAYSVLVVAKVEKGKSKKLKSVKELVGITIMERSSFEKNPIDFLEAKGYKEVRKDLIIKL |
| | PKYSLFELENGRKRMLASAGELQKGNELALPSKYVNFLYLASHYEKLKGSPEDNEQKQLFVEQHK |
| | HYLDEIIEQISEFSKRVILADANLDKVLSAYNKHRDKPIREQAENIIHLFTLTNLGAPAAFKYFDTTID |
| | RKRYTSTKEVLDATLIHQSITGLYETRIDLSQLGGD |
| 262 | MKKPYSIGLDIGTNSVGWAVVTDDYKVPAKKMKVLGDTDKSHIKKNLLGTLLFESGETAEGRRLK |
| | RTARRRYTRRRNRILYLQEIFAEEMNKIDDSFFQRLDESFLKDDDKNFDSHPIFGNKAEEDAYHQKF |
| | PTIYHLRQYLADSPEKADLRLVYLALAHIIKYRGHELFEGDLKSENKDVQHLFNDFVEMEDKTVEG |
| | SYLSENLPNVADVLVEKVSKSRRLENVLHYFPNEKKNGLFGNFIALALGLQPNFKTNFKLSEDAKL |
| | QFSKDTYDEDLEELLGKIGDDYADLFVAAKNLYDAILLSGILTVTDPTTKAPLSASMIDRYEKHQK |
| | DLALLKNLIKQNLTQEKYDEVFSDESKNGYAGYINGKVKQEEFYKYLKNTLSKIEGSDYFLDKIER |
| | EDFLRKQRTFDNGSIPHQIHLQEMNAIIRRQGEHYPFLKENKEKIEKILTFRIPYYVGPLARGNRDFA |
| | WLTRNSDQAIRPWNFEEIVDQASSAEDFINKMTNYDLYLPEEKVLPKHSLLYETFAVYNELTKVKF |
| | IAEGLRDYQFLDSGQKKQIVNQLFKEKRKVTEKDIIQYLHNVDGYDGIELKGIEKQFNASLSTYHDL |
| | LKIIKDKEFMDDPKNEEILENIVHTLTIFEDREMIKQRLAQYDSLFDEKVIKALTRRHYTGWGKLSA |
| | KLINGIYDKQSKKTILDYLIDDGEINRNFMQLINDDGLSFKEIIQKAQVVGKTNDVKQVVQELPGSP |
| | AIKKGILQSIKLVDELVKVMGHAPESIVIEMARENQTTARGKKNSQQRYKRIEDALKNLAPGLDSNI |
| | LKENPTDNIQLQNDRLFLYYLQNGKDMYTGEALDINQLSNYDIDHIVPQAFIKDDSLDNRVLTSSK |
| | DNRGKSDNVPSIEVVQKRKAFWQQLLDSKLISERKFNNLTKAERGGLDERDKVGFIKRQLVETRQI |
| | TKHVAQILDARFNTEVDENNQRIRKVKIITLKSNLVSNFRKEFGLYKVREINDYHHAHDAYLNAVV |
| | AKAILKKYPKLEPEFVYGDYQKYDLKRYISRSKDPKDIEKATEKYFFYSNLLNFFKEEVHYADGTII |
| | KRENIEYSNDTGEIAWNKEKDFATVRKVLSCPQVNIVKKTEVQTGGFSKESILPKRNSDKLIPRKKD |
| | WDPKKYGGFDSPTVAYSVLVVAKVEKGKSKKLKPVKELVGITIMERSSFEKDPVDFLEAKGYKEV |
| | RKDLIIKLPKYSLFELENGRKRMLASAGELQKGNELALPSKYVNFLYLASHYEKLKGSPEDNEQKQ |
| | LFVEQHKHYLDEIVEQISEFSERVILADANLDKVLSAYNNHRDKPIREQAENIIHLFTLTNLGAPAAF |
| | KFFDTTIDRKRYTSTKEILDATLIHQSITGLYETRIDLSQLGGD |
| 263 | MNKPYSIGLDIGTNSVGWSVVDDDYKVPAKKMRVFGDTDKGHIKKNLLGTLLFDSGETAESRRLK |
| | RTARRRYTRRRNRILYLQEIFAKEMAKVDESFFHRLDESFLTDDDKTFDSHPIFGNKAEEDAYHQQ |
| | FPTIYHLRKHLADSTEKADLRLVYLALAHMIKFRGHFLIEGELNAENTDVQKLFADFVGVYDRTFD |
| | DSHLSEITVDASSILTEKISKSRRLEKLIKHYPTEKKNTLFGNLVALALGLQPNFKTNFKLSEDAKLQ |
| | FSKDTYEEDLEELLGKIGDDYADLFTSAKNLYDAILLSGILTVDDESTKAPLSASMIKRYAEHHEDL |
| | EKLKEFVKANKPELYHDIFKDEAKNGYAGYIENGVKQDEFYKYLKSTLSKQDGSQYFLDKIEREDF |
| | LRKQRTFDNGSIPHQLHLKEMRSILHRQGEYYPFLKENQAKIEKILTFRIPYYVGPLARKDSRFAWA |
| | EYHSDEPITPWNFDEVVDKEKSAEKFITRMTLNDLYLPEEKVLPKHSHVYETYTVYNELTKVKYV |
| | NEQGEAKFFDANMKQEIFDHVFKENRKVTKDKLMNFLGKEFDEFRIVDITGLDKENKAFNASLGT |
| | YHDLKKILDKSFLDDKVNETIIEDIIQTLTLFEDKDMIRQRLQKYSDIFTPQQLKKLERRHYTGWGR |
| | LSYKLINGIRDKQTGKTILDYLIDDGYANRNFMQLINDDSLPFKEIIQKAQIIGDIDDIKAVVHDLPGS |
| | PAIKKGILQSVKIVDELVKVMGHNPDNIVIEMARENQTTNRGRTQSQQRLKKLQESLKELGSNILNE |
| | KKPSYVEDKVENSHLQNDRLFLYYIQNGKDMYTGDELDIDHLSDYDIDHIIPQAFIKDDSIDNRVLT |
| | SSAKNRGKSDDVPSIEIVRDRKADWMRLRKAGLISKRKFDNLTKAERGGLTEADKAGFIKRQLVET |
| | RQITKHVAQILDNRFNTERDENDKVIRDVKVITLKSNLVSQFRKDFQFYKVREINDYHHAHDAYLN |
| | AVVGTALIKKYPKLESEFVYGDYKVYDVRKMIAKSEQEIGKATAKRFFYSNIMNFFKTEVKLANG |
| | EIRKRPLIETNEETGEIVWNKEKDFATVRKVLSCPQVNIVKKTEVQTGGFSKESILPKRNSDKLIPRK |
| | NGWDPKKYGGFDSPTVAYSVLVVAKVEKGKSKKLKTVKELVGITIMERSSFEKDPVDFLEAKGYK |
| | EVQKDLIIKLPKYSLFELENGRKRMLASAGELQKGNELALPSKYVNFLYLASHYEKLKGSPEDNEQ |
| | KQLFVDQHKHYLDEIIEQISDFSKRVILADANLDKVLSAYNNHRDKPVREQAENIINLFTLTNLGAP |
| | AAFKFFDTTIDRKRYTSTKEILDATLIHQSITGLYETRIDLSQLGGD |
| 264 | MTKPYSIGLDIGTNSVGWAVITDSYKVPSKKMKVLGNTSKKYVKKNLLGVLLFESGITAEGRRLK |
| | RTARRRYTRRRNRILYLQEIFSTEMAKIDESFFQRLDDSFLVPDDKRDSKYPIFGNLVKEKTYHDEF |
| | PTIYHLRKYLADSSKKADLRLVYLALAHMIKYRGHFLIEGDFNSRNNDIKKNFQNFLDSYNAIFESD |
| | LSLENSKQLEEIVKDKISKSAKRERVLKLFPDEKSTGLFSEFLKLIVGNQADFKKHFDLEEKAPLQFS |
| | KDTYDEDLENLLGQIGDDFADLFLVAKKLYDAILLSGILTVTDPSTKAPLSASMIERYENHQKDLSA |
| | LKQFIRSNLSEKYDEVFSDQSKDGYAGYINGKTTQEAFYKYIKNLLSKFEGADYFLDKIEREDFLRK |
| | QRTFDNGSIPHQIHLQEMNAIIRRQGEHYPFLKENKEKIEKILTFRIPYYVGPLARGNRDFAWLTRNS |
| | DQAIRPWNFEEVVDKASSAEAFINKMTNFDLYLPEEKVLPKHSLLYETFAVYNELTKVKFIAEGLR |
| | DYQFLDSGQKKQIVNQLFKEKRKVTEKDIIQFLHNVDGYDGIELKGIEKQFNASLATYHDLLKIIKD |
| | KEFMDDPKNEEILENIIHTLTIFEDREMIKQRLAQYDSLFDEKVIKALTRRHYTGWGKLSAKLINGIC |
| | DKQTGKTILDYLIDDGFANRNFMQLINDDTLPFKQIIQKAQIVGDVDDIEQVVHNLPGSPAIKKGIL |
| | QSIKIIDELVKVMGHNPDNIVIEMARENQTTNRGRSQSQQRLKKLQESLKELGSNILNEEKPSYVES |
| | KVENSQLQNDRLFLYYIQNGKDMYTGEELDIDRLSDYDIDHIIPQAFIKDDSIDNRVLTSSAKNRGK |
| | SDDVPSLEVVKKRKTYWHQLLKSKLISQRKFDNLTKAERGGLTEKDKAGFIKRQLVETRQITKHV |
| | AQILDSRFNTKRDENDKVIRDVKVITLKSNLVSQFRKDFGFYKVREINDYHHAHDAYLNAVVGTA |
| | LLKKYPKLAPEFVYGEYKKYDVRKIVRSKDESSLGKATAKMFFYSNLMNFFKKEVRLSDGTVIVR |
| | PVIEYSKDTGEIAWNKEKDFATVRKVLSCPQVNIVKKTEVQTGGFSKESILPKRNSDKLIPRKKGW |
| | DPKKYGGFDSPTVAYSVLVVAKVEKGKSKKLKSVKELVGITIMERSSFEKDPIDFLEAKGYKEVRK |
| | DLIIKLPKYSLFELENGRKRMLASAGELQKGNELALPSKYVNFLYLASHYEKLKGSPEDNEQKQLF |
| | VEQHKHYLDEIIEQISEFSERVILADANLDKILSAYNKHRDKPIREQAENIIHLFTLTNLGAPAAFKFF |
| | DTTIDRKRYTSTKEVLDATLIHQSITGLYETRIDLSQLGGD |
| 265 | MTKPYSIGLDIGTNSVGWAVITDSYKVPSKKMKVLGNTSKKYVKKNLLGALLFESGITAEGRRLK |
| | RTARRRYTRRRNRILYLQEIFSTEMAKIDESFFQRLDDSFLVPDDKRDSKYPIFGNLVKEKTYHDEF |
| | PTIYHLRKYLADSSKKADLRLVYLALAHMIKYRGHFLIEGDFNSRNNDIKKNFQNFLDSYNAIFESD |
| | LSLENSKQLEEIVKDKISKSAKRERVLKLFPDEKSTGLFSEFLKLIVGNQADFKKHFDLEEKAPLQFS |
| | KDTYDEDLENLLGQIGDDFADLFLVAKKLYDAILLSGILTVTDPSTKAPLSASMIERYENHQKDLSA |
| | LKQFIRSNLSEKYDEVFSDQSKDGYAGYINGKTTQEAFYKYIKNLLSKFEGADYFLDKIEREDFLRK |
| | QRTFDNGSIPHQIHLQEMNAIIRRQGEHYPFLKENKEKIEKILTFRIPYYVGPLARGNRDFAWLTRNS |
| | DQAIRPWNFEEVVDKASSAEAFINKMTNFDLYLPEEKVLPKHSLLYETFAVYNELTKVKFIAEGLR |
| | DYQFLDSGQKKQIVNQLFKEKRKVTEKDIIQFLHNVDGYDGIELKGIEKQFNASLATYHDLLKIIKD |
| | KEFMDDPKNEEILENIIHTLTIFEDREMIKQRLAQYDSLFDEKVIKALTRRHYTGWGKLSAKLINGIC |
| | DKQTGKTILDYLIDDGFANRNFMQLINDDTLPFKQIIQKAQIVGDVDDIEQVVHNLPGSPAIKKGIL |
| | QSIKIVDELVKVMGHNPDNIVIEMARENQTTNRGRSQSQQRLKKLQESLKELGSNILNEEKPSYVES |
| | KVENSQLQNDRLFLYYIQNGKDMYTGEELDIDRLSDYDIDHIIPQAFIKDDSIDNRVLTSSAKNRGK |
| | SDDVPSLEVVKKRKTYWHQLLKAKLISQRKFDNLTKAERGGLTEKDKAGFIKRQLVETRQITKHV |
| | AQILDSRFNTKRDENDKVIRDVKVITLKSNLVSQFRKDFGFYKVREINDYHHAHDAYLNAVVGTA |
| | LLKKYPKLAPEFVYGEYKKYDVRKIVRSKDESSLGKATAKMFFYSNLMNFFKKEVRLSDGTVIVR |
| | PVIEYSKDTGEIAWNKEKDFATVRKVLSCPQVNIVKKTEVQTGGFSKESILPKRNSDKLIPRKKGW |
| | DPKKYGGFDSPTVAYSVLVVAKVEKGKSKKLKSVKELVGITIMERSSFEKDPVDFLEAKGYKEVR |
| | KDLIIKLPKYSLFELENGRKRMLASAGELQKGNELALPSKYVNFLYLASHYEKLKGSPEDNEQKQL |
| | FVEQHKHYLDEIVEQISEFSERVILADANLDKVLSAYNKNRDRPIREQAENIIHLFTLTNLGAPAAFK |
| | FFNTDIDRKRYTSTKEILDATLIHQSITGLYETRIDLSQLGGD |
| 266 | MKKPYSIGLDIGTNSVGWAVVTDDYKVPAKKMKVLGDTDKSHIKKNLLGTLLFESGETAEGRRLK |
| | RTARRRYTRRRNRILYLQEIFAEEMNKIDDSFFQRLDESFLKDDDKNFDSHPIFGNKAEEDAYHQKF |
| | PTIYHLRQYLADSPEKADLRLVYLALAHIIKYRGHELFEGDLKSENKDVQHLFNDFVEMEDKTVEG |
| | SYLSENLPNIADVLVEKVSKSRRLENVLHYFPNEKKNGLFGNFIALALGLQPNFKTNFKLSEDAKL |
| | QFSKDTYDEDLEELLGKIGDDYADLFVAAKNLYDAILLSGILTVTDPTTKAPLSASMIDRYEKHHK |
| | DLALLKNLIKQNLTQEKYDEVFSDESKNGYAGYINGKVKQEEFYKYLKNTLSKIEGSDYFLDKIER |
| | EDFLRKQRTFDNGSIPHQIHLQEMNAIIRRQGEHYPFLKENKEKIEKILTFRIPYYVGPLARGNRDFA |
| | WLTRNSDQAIRPWNFEEIVDQASSAEDFINKMTNYDLYLPEEKVLPKHSLLYETFAVYNELTKVKF |
| | IAEGLRDYQFLDSGQKKQIVNQLFKEKRKVTEKDIIQYLHNVDGYDGIELKGIEKQFNASLSTYHDL |
| | LKIIKDKEFMDDPKNEEILENIVHTLTIFEDREMIKQRLAQYDSLFDEKVIKALTRRHYTGWGKLSA |
| | KLINGIYDKQSKKTILDYLIDDGEINRNFMQLINDDGLSFKEIIQKAQVVGKTNDVKQVVQELPGSP |
| | AIKKGILQSIKLVDELVKIMGHAPESIVIEMARENQTTARGKKNSQQRYKRIEDALKNLAPGLDSNI |
| | LKENPTDNIQLQNDRLFLYYLQNGKDMYTGEALDINQLSNYDIDHIVPQAFIKDDSLDNRVLTSSK |
| | DNRGKSDNVPSIEVVQKRKAFWQQLLDSKLISERKENNLTKAERGGLDERDKVGFIKRQLVETRQI |
| | TKHVAQILDARFNTEVDENNQRIRKVKIITLKSNLVSNFRKEFGLYKVREINDYHHAHDAYLNAVV |
| | AKAILKKYPKLEPEFVYGDYQKYDLKRYISRSKDPKDIEKATEKYFFYSNLLNFFKEEVHYADGTII |
| | KRENIEYSNDTGEIAWNKEKDFAIVRKVLSCPQVNIVKKTEVQTGGFSKESILPKRNSDKLIPRKKN |
| | WDPKKYGGFDSPTVAYSVLVVAKVEKGKSKKLKPVKELVGITIMERSSFEKNPIDFLEAKGYKEV |
| | RKDLIIKLPKYSLFELENGRKRMLASAGELQKGNELALPSKYVNFLYLASHYEKLKGSPEDNEQKQ |
| | LFVEQHKHYLDEIIEQISEFSERVILADANLDKVLSAYNKHRDKPIREQAENIIHLFTLTNLGAPAAF |
| | KFFDTTIDRKRYTSTKEVLNATLIHQSITGLYETRIDLSKLGGD |
| 267 | MKKPYSIGLDIGTNSVGWAVVTDDYKVPAKKMKVLGNTNKESIKKNLIGALLFDAGNTAADRRL |
| | KRTARRRYTRRRNRILYLQEIFAEEMNKVDESFFHRLDDSFLVPEDKRGSKYPIFATLEEEKEYHKK |
| | FPTIYHLRKHLADSKEKADLRLIYLALAHMIKYRGHFLYEESFDIKNNDIQKIFNEFISIYDNTFEGSS |
| | LSGQNAQVEAIFTDKISKSAKRERVLKLFPDEKSTGLFSEFLKLIVGNQADFKKHFDLEEKAPLQFS |
| | KDTYDEDLENLLGQIGDDFADLFLVAKKLYDAILLSGILTVTDPSTKAPLSASMIERYENHQKDLA |
| | ALKQFIQNNLQEKYDEVFSDQSKDGYAGYIDGKTTQEAFYKYIKNLLSKLEGADYFLDKIEREDFL |
| | RKQRTFDNGSIPHQIHLQEMNAIIRRQGEHYPFLKENKEKIEKILTFRIPYYVGPLARGNRDFAWLTR |
| | NSDQAIRPWNFEEIVDQASSAEDFINKMTNYDLYLPEEKVLPKHSLLYETFAVYNELTKVKFIAEG |
| | MRDYQFLDSGQKKKIVSQLFEDKRKVTEKDIIHYLHTIDGYDGIELSGIEKQFNASLSTYHDLLKIIK |
| | DKAFLDDSENEAIIEEIIHTLTIFEDREMIKQRLAQYADIFDKSVLKKLARRHYTGWGKLSAKLINGI |
| | RDKQSNKTILDYLIDDGKINRNFMQLINDDSLSFKETIQKAQVVGDTDDVKQVVQELPGSPAIKKGI |
| | LQSIKIVDELVKVMGHNPDNIVIEMARENQTTNRGRSQSQQRLKKLQDSLKELGSNILNEEKPSYIE |
| | DKVENSHLQNDQLFLYYIQNGKDMYTGEELDIDHLSDYDIDHIIPQAFIKDDSIDNRVLTSSAKNRG |
| | KSDDVPSLDIVRERKADWIRLYKSKLISKRKFDNLTKAERGGLTEADKAGFIKRQLVETRQITKHV |
| | AQILDSRFNTKRDENDKVIRDVKVITLKSNLVSQFRKDFELYKVREINDYHHAHDAYLNAVVGTA |
| | LLKKYPKLAPEFVYGEYKKYDIRKFITSSKDKATAKYFFYSNLMNFFKRIIRYSNGKIVVRPVVEYS |
| | KDTGEIAWNKEKDFATVRKVLSCPQVNIVKKTEVQTGGFSKESILPKRNSDKLIPRKKGWDPKKY |
| | GGFDSPTVAYSVLVVAKVEKGKSKKLKSVKELVGITIMERSSFEKDPVDFLEAKGYKEVRKDLIIK |
| | LPKYSLFELENGRKRMLASAGELQKGNELALPSKYVNFLYLASHYEKLKGSPEDNEQKQLFVEQH |
| | KHYLDEIVEQISEFSERVILADANLDKVLSAYNKNRDRPIREQAENIIHLFTLTNLGAPAAFKFENTD |
| | IDRKRYTSTKEILDATLIHQSITGLYETRIDLSQLGGD |
| 268 | MKKSYSIGLDIGTNSVGWSVITDDYKVPAKKMKVLGNTDKKYIKKNLLGALLFDSGETAEATRLK |
| | RTARRRYTRRKNRLRYLQEIFAKEMAKVDESFFQRLEESFLTDDDKNFDSHPIFGNKAEEDAYHDE |
| | FPTIYHLRQYLADSPQKADLRLVYLALAHMIKFRGHFLIEGQLNAENTDVQTLFNDFVEVYDKTVE |
| | ESHLSEITVDALSILTEKVSKSRRLENLVKCYPTEKKNTLFGNLIALSLGLQPNFKTNFKLSEDAKLQ |
| | FSKDTYEEDLEELLGKIGDEYADLFASAKNLYDAILLSGILTVDDNTTKAPLSASMVKRYAEHHED |
| | LEKLKEFVKANKPELYHDIFKDETKNGYAGYIENGVKQDEFYKYLKNTLSKIAGSDYFLDKIERED |
| | FLRKQRTFDNGSIPHQIHLQEMHAILRRQGEYYPFLKENQAKIEKILTFRIPYYVGPLARKNSRFAW |
| | AEYHSDEKITPWNFEEIVDKEESATAFIHRMTNNDLYLPEEKVLPKHSLIYEEFTVYNELTKVKYVT |
| | DQGKAYYFDANMKQEIFDGVFKEHRKVTKDKLMNFLEKEFDEFRIVDLTGLDKENKSFNASLGTY |
| | HDLKKILDKSFLDDKTNETIIEDIIQTLTLFEDRDMIRQRLQKYSDIFTPQQLKKLERRHYTGWGRLS |
| | YKLINGIRNKENGKTILDYLIDDGYANRNFMQLINDDTLPFKQIIQEAQIIGDIDDIETVVHDLPGSPA |
| | IKKGILQSVKIVDELVKVMGHNPDNIVIEMARENQTTNRGRSQSQQRLKKLQESLKELGSNILNEEK |
| | PSYVEDKVENSHLQNDKLFLYYIQNGKDMYTGEELDIDHLSDYDIDHIIPQAFIKDNSIDNRVLTSS |
| | AKNRGKSDDVPSLDIVHDRKADWIRLYKSGLISKRKFDNLTKAEHGGLTEDDKAGFIKRQLVETR |
| | QITKHVAQILDSRENTERDENDKVIRDVKVITLKSNLVSQFRKDFELHKVREINDYHHAHDAYLNA |
| | VIGNALLVKYPQLEPEFVYGKFKHFHGHKENKATAKKFFYSNIMALFKKDVEYSDDTGEMVWNK |
| | EKDFATVRKVLSCPQVNIVKKTEVQTGGFSKESILPKRNSDKLIARKKDWDPKKYGGFDSPTVAYS |
| | VLVVAKVEKGKSKKLKSVKELVGITIMERSSFEKDPIDFLEAKGYKEVRKDLIIKLPKYSLFELENG |
| | RKRMLASAGELQKGNELALPSKYVNFLYLASHYEKLKGSPEDNEQKQLFVEQHKHYLDEIIEQISE |
| | FSKRVILADANLDKVLSAYNKHRDKPIREQAENIIHLFTLTNLGAPAAFKYFDTTIDRKRYTSTKEV |
| | LDSTLIHQSITGLYETRIDLSQLGGD |
| 269 | MTKPYSIGLDIGTNSVGWAVITDSYKVPSKKMKVLGNTSKKYVKKNLLGALLFESGITAEGRRLK |
| | RTARRRYTRRRNRILYLQEIFSTEMAKIDESFFQRLDDSFLVPDDKRDSKYPIFGNLVKEKTYHDEF |
| | PTIYHLRKYLADSSKKADLRLVYLALAHMIKYRGHFLIEGDFNSRNNDIKKNFQNFLDSYNAIFESD |
| | LSLENSKQLEEIVKDKISKSAKRERVLKLFPDEKSTGLFSEFLKLIVGNQADFKKHFDLEEKAPLQFS |
| | KDTYDEDLENLLGQIGDDFADLFLVAKKLYDAILLSGILTVTDPSTKAPLSASMIERYENHQKDLSA |
| | LKQFIRSNLSEKYDEVFSDQSKDGYAGYINGKTTQEAFYKYIKNLLSKFEGADYFLDKIEREDFLRK |
| | QRTFDNGSIPHQIHLQEMNAIIRRQGEHYPFLKENKEKIEKILTFRIPYYVGPLARGNRDFAWLTRNS |
| | DQAIRPWNFEEVVDKASSAEAFINKMTNFDLYLPEEKVLPKHSLLYETFAVYNELTKVKFIAEGLR |
| | DYQFLDSGQKKQIVNQLFKEKRKVTEKDIIQFLHNVDGYDGIELKGIEKQFNASLATYHDLLKIIKD |
| | KEFMDDPKNEEILENIIHTLTIFEDREMIKQRLAQYDSLFDEKVIKALTRRHYTGWGKLSAKLINGIC |
| | DKQTGKTILDYLIDDGFANRNFMQLINDDTLPFKQIIQKAQIVGDVDDIEQVVHNLPGSPAIKKGIL |
| | QSIKIVDELVKVMGHNPDNIVIEMARENQTTNRGRSQSQQRLKKLQESLKELGSNILNEEKPSYIES |
| | KVENSHLQNDRLFLYYIQNGKDMYTGEELDIDRLSDYDIDHIIPQAFIKDDSIDNRVLTSSAKNRGK |
| | SDDVPSLEVVRKRKTYWHQLLKSKLISQRKFDNLTKAERGGLTEKDKAGFIKRQLVETRQITKHV |
| | AQILDSRFNTKRDENDKVIRDVKVITLKSNLVSQFRKDFGFYKVREINDYHHAHDAYLNAVVGTA |
| | LLKKYPKLAPEFVYGEYKKYDVRKIVRSKDESSLGKATAKMFFYSNLMNFFKKEVRLSDGTVIVR |
| | PVIEYSKDTGEIAWNKEKDFATVRKVLSCPQVNIVKKTEVQTGGFSKESILPKRNSDKLIPRKKNW |
| | DPKKYGGFDSPTVAYSVLVVAKVEKGKSKKLKPVKELVGITIMERSSFEKDPVDFLEAKGYKEVR |
| | EDLIIKLPKYSLFELENGRKRLLASAGELQKGNELALPSKYVNFLYLASHYEKLKGSPEDNEQKQLF |
| | VEQHKHYLDEIVEQISEFSERVILADANLDKILSAYNKHRDKPIREQAENIIHLFTLTNLGAPAAFKF |
| | FDTTIDRKRYTSTKEVLDATLIHQSITGLYETRIDLSKLGGD |
| 270 | MKKPYSIGLDIGTNSVGWAVVDDDYKVPAKKMRVFGDTDRSHIKKNLLGTLLFDDGNTAESRRL |
| | KRTARRRYTRRRNRILYLQEIFAEEMNKVDESFFHRLDDSFLVPEDKRGSKYPIFATLEEEKEYHKK |
| | FPTIYHLRKHLADSKEKTDLRLIYLALAHMIKYRGHFLYEESFDIKNNDIQKIFNEFISLYDNTFEGSS |
| | LSGQNAQVEAIFTDKISKSAKRERVLKLFPDEKSTGLFSEFLKLIVGNQADFKKHFDLEEKAPLQFS |
| | KDTYDEDLENLLGQIGDDFADLFLVAKKLYDAILLSGILTVTDPSTKAPLSASMIERYENHEEDLAA |
| | LKQFVKANLPEKYDEVFSDQSKDGYAGYIDGKTTQEAFYKYIKNLLSKLEGADYFLDKIEREDFLR |
| | KQRTFDNGSIPHQIHLQEMNAILRRQGEHYPFLKDNKEKIEKILTFRIPYYVGPLARGNRDFAWLTR |
| | NSDQAIRPWNFEEVVDKASSAEDFINKMTNYDLYLPEEKVLPKHSLLYETFAVYNELTKVKFIAEG |
| | LRDYQFLDSGQKKQIVNQLFKTKRKVTEKDIINYLHKVDGYDGVEISGVEDRFNASLGTYHDLLKI |
| | IKDKSFLDDKKNEEIIEDIVLTLTLFEDRDMIRQRLQKYSDIFTPQQLKKLERRHYTGWGRLSYKLIN |
| | GIRNKENNKSILDYLIDDGYANRNFMQLINDDTLPFKQIIQKSQVVGDIDDIKTVVHDLPGSPAIKK |
| | GILQSVKIVDELVKVMGHNPDNIVIEMARENQTTNRGRSQSQQRLKKLQESLKELGSNILNVEKPS |
| | YIEDKVENNHLQNDKLFLYYIQNGKDMYTGEELDIDHLSDYDIDHIIPQAFIKDDSIDNRVLTSSAK |
| | NRGKSDDVPSLDIVRGRKAEWIRLYKAKLISQRKFDNLTKAERGGLTENDKAGFIKRQLVETRQIT |
| | KHVAQILDARFNTKRDENDKLIREVKVITLKSNLVSQFRKEFGFYKVREINDYHHAHDAYLNAVV |
| | GTAILKKYPKLTSEFVYGEYKKYDIRKFIKSDDHSEMGKATAKYFFYSNLLNFFKEEIKYANGKIVV |
| | RPVVEYSKDTGEIAWNKEKDFATVRKVLSCPQVNIVKKTEVQTGGFSKESILPKRNSDKLIARKKD |
| | WDPKKYGGFDSPTVAYSVLVVAKVEKGKSKKLKSVKELVGITIMERSSFEKDPIDFLEAKGYKEV |
| | RKDLIIKLPKYSLFELENGRKRMLASAGELQKGNELALPSKYVNFLYLASHYEKLKGSPEDNEQKQ |
| | LFVEQHKHYLDEIIEQISEFSKRVILADANLDKVLSAYNKHRDKPIREQAENIIHLFTLTNLGAPAAF |
| | KYFDTTIDRKRYTSTKEVLDATLIHQSITGLYETRIDLSQLGGD |
| 271 | MKKPYSIGLDIGTNSVGWAVVTDDYKVPSKKMKVLGNTDKSHIKKNLLGALLFESGETAEGRRLK |
| | RTARRRYTRRRNRILYLQEIFAEEMNKIDESFFHRLDDSFLVPEDKRGSKYPIFATLQEEKEYHKQFP |
| | TIYHLRKQLAESNEKADLRLVYLALAHMIKYRGHFLIDDPKFKVQNNDIQGLFEKFVEEYDNVQET |
| | SLSKIKLNVTEILTANIPKSEKQEQLLKNYPSEKKNTLFGNLIGLALGLTPNFKTNFSLENDAKLQISS |
| | ESYEEDLGNLLALIGENFIELFSAVKNLSDGILLAGIVSDESPHAPLSTKMVIRFKEHEEDLAALKHFI |
| | KANLPEKYDEVFSDDSKNGYAGYVGVDSKVRKRNGKLATEEEFYKYLKDILNNVKGADYFLEKI |
| | KREDFLRKQRTFDNGTIPYQVHLTEMRAIIHRQGEHYPFLQENKEKIEKILTFRIPYYVGPLARGNR |
| | DFAWLTRNSDQAIRPWNFEEVVDKASSAEDFINKMTNYDLYLPEEKVLPKHSLLYETFAVYNELT |
| | KVKFIAEGLRDYQFLDSGQKKQIVNQLFKEKRKVTEKDIIHYLHNVDGYDGIELKGIEKQFNASLST |
| | YHDLLKIIKDKEFMDDAKNEEILENIVHTLTIFEDREMIKQRLAQYDSLFDEKVIKALTRRHYTGWG |
| | KLSAKLINGIYDKQSKKTILDYLIDDDKINRNFMQLINDDGLSFKDIIQKAQVVGKTNDVKQVVQE |
| | LPGSPAIKKGILQSIKLVDELVKVMGHAPESIVIEMARENQTTARGKKNSQQRYKRIEDALKNLAPG |
| | LDSNILKENPTDNIQLQNDRLFLYYLQNGKDMYTGEPLDINQLSSYDIDHIVPQAFIKDDSLDNRVL |
| | TSSKDNRGKSDNVPSLEVVQKRKAFWQQLLDSKLISEHKFNNLTKAERGGLDERDKVGFIKRQLV |
| | ETRQITKHVAQILDARFNTEVNEKKQKIRSVKIITLKSNLVSNFRKEFRLYKVREINDYHHAHDAYL |
| | NAVVAKAILKKYPKLEPEFVYGDYQKYDIKRYISRSKDPKEVEKATQKYFFYSNLLNFFKEEVHYA |
| | DGTIIKRENIEYSKDTGEIAWNKEKDFATVRKVLSCPQVNIVKKTEVQTGGFSKESILPKRNSDKLIP |
| | RKTEWDPKKYGGFDSPIVAYSVLVVAKVEKGKSKKLKPVKELVGITIMERSSFEKDPVDFLEAKG |
| | YKEVRKDLIIKLPKYSLFELENGRKRMLASAGELQKGNELALPSKYVNFLYLASHYEKLKGSPEDN |
| | EQKQLFVEQHKHYLDEIVEQISEFSERVILADANLDKILSAYNKHRDKPIREQAENIINLFTFTNLGA |
| | PAAFKFFDTDIDRKRYTSTKEVLDATLIHQSITGLYETRIDLSQLGGD |
| 272 | MKKSYSIGLDIGTNSVGWAVVDDDYKVPAKKMRVFGDTDRSHIKKNLLGTLLFDEGNTAESRRL |
| | KRTARRRYTRRRNRILYLQEIFATEMNKVDESFFHRLDDSFLVPEDKRGSKYPIFATLEEEKEYHKQ |
| | FPTIYHLRKQLAESNEKADLRLVYLALAHMIKYRGHFLIDDPKFKVQNNDIQGLFEKFVEEYDNVQ |
| | ETSLSKIKLNVTEILTANIPKSEKQEQLLKNYPSEKKNTLFGNLIGLALGLTPNFKTNFSLENDAKLQI |
| | SSESYEEDLGNLLALIGENFIELFSAVKNLSDGILLAGIVSDESPHAPLSAKMVIRFKEHEEDLAALK |
| | HFIKANLPEKYDEVFSDDSKNGYAGYVGVDSKVRKRNGKLATEEEFYKYLKEILNNVKGADYFLE |
| | KIKREDFLRKQRTFDNGTIPYQVHLTEMRAILHRQGEYYPFLKENKEKIEKILTFRIPYYVGPLARG |
| | NRDFAWLTRNSDQAIRPWNFEEVVDKASSAEDFINKMTNYDLYLPEEKVLPKHSLLYETFAVYNE |
| | LTKVKFIAEGLRDYQFLDSGQKKQIVNQLFKEKRKVTEKDIIHYLHNVDGYDGIELKGIEKQFNASL |
| | STYHDLLKIIKDKEFMDDPKNEEILENIVHTLTIFEDREMIKQRLAQYDSLFDEKVIKALTRRHYTG |
| | WGKLSAKLINGIYDKQSKKTILDYLIDDDKINRNFMQLINDDGLSFKEIIQKAQVVGKTDDVKQVV |
| | QELPGSPAIKKGILQSIKIVDELVKVMGHAPESIVIEMARENQTTARGKKNSQQRYKRIEDALKNLA |
| | PGLDSNILKENPTDNIQLQNDRLFLYYLQNGKDMYTGEELDINQLSNYDIDHIVPQAFIKDDSLDNR |
| | VLTSSKDNRGKSDNVPSLEVVQKRKAFWQQLLDSKLISERKENNLTKAERGGLDERDKVGFIRRQ |
| | LVETRQITKHVAQILDARFNTEVTENNQKIRKVKIITLKSNLVSNFRKEFGLYKVREINDYHHAHDA |
| | YLNAVVAKAILKKYPKLEPEFVYGDYQKYDIKRYISRSKDPKEVEKATEKYFFYSNLLNFFKEEVH |
| | YADGTIVKRENIEYSKDTGEIAWNKEKDFATVRKVLSCPQVNIVKKTEVQTGGFSKESILPKRNSD |
| | KLIARKKDWDPKKYGGFDSPTVAYSVLVVAKVEKGKSKKLKSVKELLGITIMERSSFEKNPIDFLE |
| | AKGYKEVRKDLIIKLPKYSLFELENGRKRMLASAGELQKGNELALPSKYVNFLYLASHYEKLKGSP |
| | EDNEQKQLFVEQHKHYLDEIIEQISEFSKRVILADANLDKVLSAYNKHRDKPIREQAENIIHLFTLTN |
| | LGAPAAFKYFDTTIDRKRYTSTKEVLDATLIHQSITGLYETRIDLSQLGGD |
| 273 | MDKKYSIGLDIGTNSVGWAVITDDYKVPAKKMKVLGNTDKRTIKKNLLGALLFDSGETAEATRLK |
| | RTARRRYTRRKNRLRYLQEIFSKEMSNVDANFFYRLEDSFLVPEDKQQERHPIFGNLVEEIEYHKEF |
| | PTIYHLRKQLADSKEKADLRLIYLALAHMIKYRGHFLFEGDLKSENTDVQRLFKDFVEVYDKTVEG |
| | SYLSENLTNVTDVLVEKISKSRRLENILHYFPNEKKNGLFGNFLALALGLQPNFKNNFDLAEEAKL |
| | QISKDTYEEELEVLLAQIGDNYAELFIATKELYDGILLAGILTVTDSSTKAPLSASMIERYENHQGDL |
| | AKLKAFIRRFCKETYDEVFSDEVKDGYAGYIEGKTSQEDFYVHLKKLLANLEGADYFLEKINREDL |
| | LRKQRTFDNGSIPYQIHLVEMRAILDKQAKFYPFLAKNQDRIEKLLTFRIPYYVGPLARGKSDFAWL |
| | SRKSADKITPWNFDEIVDKESSAEAFINRMTNYDLYLPNQKVLPKHSLLYEKFTVYNELTKVKYKT |
| | EQGKTAFFDANMKQEIFDGVFKVYRKVTKDKLMDFLEKEFDEFRIVDLMGLDKENKAFNASYGT |
| | YHDLRKILDKDFLDNSDNEKILEDIVLTLTLFEDREMIRKRLEKYKDIFTEKQRKQLERRHYTGWG |
| | RLSAKLIHGIRNKESRKTILDYLIDDGYSNRNFMQLINDDTLTFKEEIAKAQVVGETDSLKQFISELA |
| | GSPAIKKGILQTVKIVDELVKVMGHKPENIVIEMARENQTTQKGQKNSQERMKRIEEGIKELGSQIL |
| | KEHPVENTQLQNEKLYLYYLQNGRDMYVDQELDINRLSDYDIDHIIPQAFIKDDSIDNRVLTSSKD |
| | NRGKSDNVPSEDVVKKMKNYWRQLLNAKLISQRKFDNLTKAERGGLSELDKVGFIKRQLVETRQI |
| | TKHVAQILDSRMNTKYDENDKLIRDVKIITLKSKLVSDFRKDFQFYKVREINNYHHAHDAYLNAV |
| | VGTALIKKYPKLESEFVYGDYKVYDVQKMIAKSEQEIGKATAKFFFYSNIMNFFKTEITLANGEIRK |
| | RPMIETNEETGEIVWDKGRDFATVRKVLCMPQVNIVKKTEVQTGGFSKESILPKRNSDKLIARKKD |
| | WDPKKYGGFDSPTVAYSVLVVAKVEKGKSKKLKSVKELVGITIMERSSFEKDPVDFLEAKGYKEV |
| | RKDLIIKLPKYSLFELENGRKRMLASAGELQKGNELALPSKYVNFLYLASHYEKLKGSPEDNEQKQ |
| | LFVEQHQHYLDEIIEQISEFSKRVILADANLDKVLSAYNKHRDKPIREQAENIIHLFTLTNLGAPAAF |
| | KYFDTTIDRKRYTSTKEVLDATLIHQSITGLYETRIDLSQLGGD |
| 274 | MKKPYSIGLDIGTNSVGWAVVTDDYKVPAKKMKVLGDTDKSHIKKNLLGTLLFESGETAEGRRLK |
| | RTARRRYTRRRNRILYLQEIFAEEMNKVDDSFFQRLDESFLKDDDKNFDSHPIFGNKAEEDAYHQK |
| | FPTIYHLRQYLADSPEKADLRLVYLALAHIIKYRGHELFEGDLKSENKDVQHLFNDFVEMFDKTVE |
| | GSYLSENLPNVADVLVEKVSKSRRLENVLHYFPNEKKNGLFGNFIALALGLQPNFKTNFKLSEDAK |
| | LQFSKDTYDEDLEELLGKIGDDYADLFVAAKNLYDAILLSGILTVTDPTTKAPLSASMIDRYEKHH |
| | KDLALLKNLIKQNLTQEKYDEVFSDESKNGYAGYISGKVKQEEFYKYLKNTLSKIEGSDYFLDKIE |
| | REDFLRKQRTFDNGSIPHQIHLQEMNAIIRRQGEHYPFLKENKEKIEKILTFRIPYYVGPLARGNRDF |
| | AWLTRNSDQAIRPWNFEEIVDQASSAEDFINKMTNYDLYLPEEKVLPKHSLLYETFAVYNELTKVK |
| | FIAEGLRDYQFLDSGQKKQIVNQLFKEKRKVTEKDIIQYLHNVDGYDGIELKGIEKQFNASLSTYHD |
| | LLKIIKDKEFMDDPKNEEILENIVHTLTIFEDREMIKQRLAQYDSLFDEKVIKALTRRHYTGWGKLS |
| | AKLINGIYDKQSKKTILDYLIDDGEINRNFMQLINDDGLSFKDIIQKAQVVGKTNDVKQVVQELPGS |
| | PAIKKGILQSIKLVDELVKVMGHAPESIVIEMARENQTTARGKKNSQQRYKRIEDALKNLAPGLDS |
| | NILKENPTDNIQLQNDRLFLYYLQNGKDMYTGEALDINQLSNYDIDHIVPQAFIKDDSLDNRVLTSS |
| | KDNRGKSDNVPSIEVVQKRKAFWQQLLDSKLISERKENNLTKAERGGLDERDKVGFIKRQLVETR |
| | QITKHVAQILDARFNTEVDENNQRIRKVKIITLKSNLVSNFRKEFGLYKVREINDYHHAHDAYLNA |
| | VVAKAILKKYPKLEPEFVYGDYQKYDLKRYISRSKDPKDIEKATEKYFFYSNLLNFFKEEVHYADG |
| | TIIKRENIEYSNDTGEIAWNKEKDFATVRKVLSCPQVNIVKKTEVQTGGFSKESILPKRNSDKLIARK |
| | KDWDPKKYGGFDSPTVAYSVLVVAKVEKGKSKKLKSVKELLGITIMERSSFEKNPIDFLEAKGYKE |
| | VRKDLIIKLPKYSLFELENGRKRMLASAGELQKGNELALPSKYVNFLYLASHYEKLKGSPEDNEQK |
| | QLFVEQHKHYLDEIIEQISEFSKRVILADANLDKVLSAYNKHRDKPIREQAENIIHLFTLTNLGAPAA |
| | FKYFDTTIDRKRYTSTKEVLDATLIHQSITGLYETRIDLSQLGGD |
| 275 | MTKPYSIGLDIGTNSVGWAVITDSYKVPSKKMKVLGNTSKKYVKKNLLGALLFESGITAEGRRLK |
| | RTARRRYTRRRNRILYLQEIFSTEMAKIDESFFQRLDDSFLVPDDKRDSKYPIFGNLVKEKTYHDEF |
| | PTIYHLRKYLADSSKKADLRLVYLALAHMIKYRGHFLIEGDFNSRNNDIKKNFQNFLDSYNAIFESD |
| | LSLENSKQLEEIVKDKISKSAKRERVLKLFPDEKSTGLFSEFLKLIVGNQADFKKHFDLEEKAPLQFS |
| | KDTYDEDLENLLGQIGDDFADLFLVAKKLYDAILLSGILTVTDPSTKAPLSASMIERYENHQKDLSA |
| | LKQFIRSNLSEKYDEVFSDQSKDGYAGYINGKTTQEAFYKYIKNLLSKFEGADYFLDKIEREDFLRK |
| | QRTFDNGSIPHQIHLQEMNAIIRRQGEHYPFLKENKEKIEKILTFRIPYYVGPLARGNRDFAWLTRNS |
| | DQAIRPWNFEEVVDKASSAEAFINKMTNFDLYLPEEKVLPKHSLLYETFAVYNELTKVKFIAEGLR |
| | DYQFLDSGQKKQIVNQLFKEKRKVTEKDIIQFLHNVDGYDGIELKGIEKQFNASLATYHDLLKIIKD |
| | KEFMDDPKNEEILENIIHTLTIFEDREMIKQRLAQYDSLFDEKVIKALTRRHYTGWGKLSAKLINGIC |
| | DKQTGKTILDYLIDDGFANRNFMQLINDDTLPFKQIIQKAQIVGDVDDIEQVVHNLPGSPAIKKGIL |
| | QSIKIVDELVKVMGHNPDNIVIEMARENQTTNRGRSQSQQRLKKLQESLKELGSNILNEEKPSYVES |
| | KVENSQLQNDRLFLYYIQNGKDMYTGEELDIDRLSDYDIDHIIPQAFIKDDSIDNRVLTSSAKNRGK |
| | SDDVPSLEVVKKRKTYWHQLLKAKLISQRKFDNLTKAERGGLTEKDKAGFIKRQLVETRQITKHV |
| | AQILDSRFNTKRDENDKVIRDVKVITLKSNLVSQFRKDFGFYKVREINDYHHAHDAYLNAVVGTA |
| | LLKKYPKLAPEFVYGEYKKYDVRKIVRSKDESSLGKATAKMFFYSNLMNFFKKEVRLSDGTVIVR |
| | PVIEYSKDTGEIAWNKEKDFATVRKVLSCPQVNIVKKTEVQTGGFSKESILPKRNSDKLIARKKDW |
| | DPKKYGGFDSPTVAYSVLVVAKVEKGKSKKLKSVKELVGITIMERSSFEKNPIDFLEAKGYKEVRK |
| | DLIIKLPKYSLFELENGRKRMLASAGELQKGNELALPSKYVNFLYLASHYEKLKGSPEDNEQKQLF |
| | VEQHKHYLDEIIEQISEFSKRVILADANLDKVLSAYNKHRDKPIREQAENIIHLFTLTNLGAPAAFKY |
| | FDTTIDRKRYTSTKEVLDATLIHQSITGLYETRIDLSQLGGD |
| 276 | MKKPYSIGLDIGTNSVGWAVVTDDYKVPSKKMKVLGNTDKSHIKKNLLGALLFESGETAEGRRLK |
| | RTARRRYTRRRNRILYLQEIFAEEMNKIDESFFHRLDDSFLVPEDKRGSKYPIFATLQEEKEYHKQFP |
| | TIYHLRKQLAESNEKADLRLVYLALAHMIKYRGHFLIDDPKFKVQNNDIQGLFEKFVEEYDNVQET |
| | SLSKIKLNVTEILTANIPKSEKQEQLLKNYPSEKKNTLFGNLIGLALGLTPNFKTNFSLENDAKLQISS |
| | ESYEEDLGNLLALIGENFIELFSAVKNLSDGILLAGIVSDESPHAPLSTKMVIRFKEHEEDLAALKHFI |
| | KANLPEKYDEVFSDDSKNGYAGYVGVDSKVRKRNGKLATEEEFYKYLKDILNNVKGADYFLEKI |
| | KREDFLRKQRTFDNGTIPYQVHLTEMRAIIHRQGEHYPFLQENKEKIEKILTFRIPYYVGPLARGNR |
| | DFAWLTRNSDQAIRPWNFEEVVDKASSAEDFINKMTNYDLYLPEEKVLPKHSLLYETFAVYNELT |
| | KVKFIAEGLRDYQFLDSGQKKQIVNQLFKEKRKVTEKDIIHYLHNVDGYDGIELKGIEKQFNASLST |
| | YHDLLKIIKDKEFMDDAKNEEILENIVHTLTIFEDREMIKQRLAQYDSLFDEKVIKALTRRHYTGWG |
| | KLSAKLINGIYDKQSKKTILDYLIDDDKINRNFMQLINDDGLSFKDIIQKAQVVGKTNDVKQVVQE |
| | LPGSPAIKKGILQSIKLVDELVKVMGHAPESIVIEMARENQTTARGKKNSQQRYKRIEDALKNLAPG |
| | LDSNILKENPTDNIQLQNDRLFLYYLQNGKDMYTGEPLDINQLSSYDIDHIVPQAFIKDDSLDNRVL |
| | TSSKDNRGKSDNVPSLEVVQKRKAFWQQLLDSKLISEHKFNNLTKAERGGLDERDKVGFIKRQLV |
| | ETRQITKHVAQILDARFNTEVNEKKQKIRSVKIITLKSNLVSNFRKEFRLYKVREINDYHHAHDAYL |
| | NAVVAKAILKKYPKLEPEFVYGDYQKYDIKRYISRSKDPKEVEKATQKYFFYSNLLNFFKEEVHYA |
| | DGTIIKRENIEYSKDTGEIAWNKEKDFATVRKVLSCPQVNIVKKTEVQTGGFSKESILPKRNSDKLIA |
| | RKKDWDPKKYGGFDSPTVAYSVLVVAKVEKGKSKKLKSVKELLGITIMERSSFEKNPIDFLEAKG |
| | YKEVRKDLIIKLPKYSLFELENGRKRMLASAGELQKGNELALPSKYVNFLYLASHYEKLKGSPEDN |
| | EQKQLFVEQHKHYLDEIIEQISEFSKRVILADANLDKVLSAYNKHRDKPIREQAENIIHLFTLTNLGA |
| | PAAFKYFDTTIDRKRYTSTKEVLDATLIHQSITGLYETRIDLSQLGGD |
| 277 | MKKPYSIGLDIGTNSVGWAVVTDDYKVPAKKMKVLGNTNKESIKKNLIGALLFDAGNTAADRRL |
| | KRTARRRYTRRRNRILYLQEIFAEEMNKVDESFFHRLDDSFLVPEDKRGSKYPIFATLEEEKEYHKK |
| | FPTIYHLRKHLADSKEKADLRLIYLALAHMIKYRGHFLYEESFDIKNNDIQKIFNEFISIYDNTFEGSS |
| | LSGQNAQVEAIFTDKISKSAKRERVLKLFPDEKSTGLFSEFLKLIVGNQADFKKHFDLEEKAPLQFS |
| | KDTYDEDLENLLGQIGDDFADLFLVAKKLYDAILLSGILTVTDPSTKAPLSASMIERYENHQKDLA |
| | ALKQFIQNNLQEKYDEVFSDQSKDGYAGYIDGKTTQEAFYKYIKNLLSKLEGADYFLDKIEREDFL |
| | RKQRTFDNGSIPHQIHLQEMNAIIRRQGEHYPFLKENKEKIEKILTFRIPYYVGPLARGNRDFAWLTR |
| | NSDQAIRPWNFEEIVDQASSAEDFINKMTNYDLYLPEEKVLPKHSLLYETFAVYNELTKVKFIAEG |
| | MRDYQFLDSGQKKKIVSQLFEDKRKVTEKDIIHYLHTIDGYDGIELSGIEKQFNASLSTYHDLLKIIK |
| | DKAFLDDSENEAIIEEIIHTLTIFEDREMIKQRLAQYADIFDKSVLKKLARRHYTGWGKLSAKLINGI |
| | RDKQSNKTILDYLIDDGKINRNFMQLINDDSLSFKETIQKAQVVGDTDDVKQVVQELPGSPAIKKGI |
| | LQSIKIVDELVKVMGHNPDNIVIEMARENQTTNRGRSQSQQRLKKLQDSLKELGSNILNEEKPSYIE |
| | DKVENSHLQNDQLFLYYIQNGKDMYTGEELDIDHLSDYDIDHIIPQAFIKDDSIDNRVLTSSAKNRG |
| | KSDDVPSLDIVRERKADWIRLYKSKLISKRKFDNLTKAERGGLTEADKAGFIKRQLVETRQITKHV |
| | AQILDSRFNTKRDENDKVIRDVKVITLKSNLVSQFRKDFELYKVREINDYHHAHDAYLNAVVGTA |
| | LLKKYPKLAPEFVYGEYKKYDIRKFITSSKDKATAKYFFYSNLMNFFKRIIRYSNGKIVVRPVVEYS |
| | KDTGEIAWNKEKDFATVRKVLSCPQVNIVKKTEVQTGGFSKESILPKRNSDKLIARKKDWDPKKY |
| | GGFDSPTVAYSVLVVAKVEKGKSKKLKSVKELLGITIMERSSFEKNPIDFLEAKGYKEVRKDLIIKL |
| | PKYSLFELENGRKRMLASAGELQKGNELALPSKYVNFLYLASHYEKLKGSPEDNEQKQLFVEQHK |
| | HYLDEIIEQISEFSKRVILADANLDKVLSAYNKHRDKPIREQAENIIHLFTLTNLGAPAAFKYFDTTID |
| | RKRYTSTKEVLDATLIHQSITGLYETRIDLSQLGGD |
| 278 | MKKSYSIGLDIGTNSVGWSVITDDYKVPAKKMKVLGNTDKKYIKKNLLGALLFDSGETAEATRLK |
| | RTARRRYTRRKNRLRYLQEIFAKEMAKVDESFFQRLEESFLTDDDKNFDSHPIFGNKAEEDAYHDE |
| | FPTIYHLRQYLADSPQKADLRLVYLALAHMIKFRGHFLIEGQLNAENTDVQTLFNDFVEVYDKTVE |
| | ESHLSEITVDALSILTEKVSKSRRLENLVKCYPTEKKNTLFGNLIALSLGLQPNFKTNFKLSEDAKLQ |
| | FSKDTYEEDLEELLGKIGDEYADLFASAKNLYDAILLSGILTVDDNTTKAPLSASMVKRYAEHHED |
| | LEKLKEFVKANKPELYHDIFKDETKNGYAGYIENGVKQDEFYKYLKNTLSKIAGSDYFLDKIERED |
| | FLRKQRTFDNGSIPHQIHLQEMHAILRRQGEYYPFLKENQAKIEKILTFRIPYYVGPLARKNSRFAW |
| | AEYHSDEKITPWNFEEIVDKEESATAFIHRMTNNDLYLPEEKVLPKHSLIYEEFTVYNELTKVKYVT |
| | DQGKAYYFDANMKQEIFDGVFKEHRKVTKDKLMNFLEKEFDEFRIVDLTGLDKENKSFNASLGTY |
| | HDLKKILDKSFLDDKTNETIIEDIIQTLTLFEDRDMIRQRLQKYSDIFTPQQLKKLERRHYTGWGRLS |
| | YKLINGIRNKENGKTILDYLIDDGYANRNFMQLINDDTLPFKQIIQEAQIIGDIDDIETVVHDLPGSPA |
| | IKKGILQSVKIVDELVKVMGHNPDNIVIEMARENQTTNRGRSQSQQRLKKLQESLKELGSNILNEEK |
| | PSYVEDKVENSHLQNDKLFLYYIQNGKDMYTGEELDIDHLSDYDIDHIIPQAFIKDNSIDNRVLTSS |
| | AKNRGKSDDVPSLDIVHDRKADWIRLYKSGLISKRKFDNLTKAEHGGLTEDDKAGFIKRQLVETR |
| | QITKHVAQILDSRENTERDENDKVIRDVKVITLKSNLVSQFRKDFELHKVREINDYHHAHDAYLNA |
| | VIGNALLVKYPQLEPEFVYGKFKHFHGHKENKATAKKFFYSNIMALFKKDVEYSDDTGEMVWNK |
| | EKDFATVRKVLSCPQVNIVKKTEVQTGGFSKESILPKRNSDKLIPRKKGWDPKKYGGFDSPTVAYS |
| | VLVVAKVEKGKSKKLKSVKELVGITIMERSSFEKDPVDFLEAKGYKEVRKDLIIKLPKYSLFELENG |
| | RKRMLASAGELQKGNELALPSKYVNFLYLASHYEKLKGSPEDNEQKQLFVEQHKHYLDEIVEQIS |
| | EFSERVILADANLDKVLSAYNKNRDRPIREQAENIIHLFTLTNLGAPAAFKFFNTDIDRKRYTSTKEI |
| | LDATLIHQSITGLYETRIDLSQLGGD |
| 279 | MKKPYSIGLDIGTNSVGWAVVDDDYKVPAKKMRVFGDTDRSHIKKNLLGTLLFDDGNTAESRRL |
| | KRTARRRYTRRRNRILYLQEIFAEEMNKVDESFFHRLDDSFLVPEDKRGSKYPIFATLEEEKEYHKK |
| | FPTIYHLRKHLADSKEKTDLRLIYLALAHMIKYRGHFLYEESFDIKNNDIQKIFNEFISLYDNTFEGSS |
| | LSGQNAQVEAIFTDKISKSAKRERVLKLFPDEKSTGLFSEFLKLIVGNQADFKKHFDLEEKAPLQFS |
| | KDTYDEDLENLLGQIGDDFADLFLVAKKLYDAILLSGILTVTDPSTKAPLSASMIERYENHEEDLAA |
| | LKQFVKANLPEKYDEVFSDQSKDGYAGYIDGKTTQEAFYKYIKNLLSKLEGADYFLDKIEREDFLR |
| | KQRTFDNGSIPHQIHLQEMNAILRRQGEHYPFLKDNKEKIEKILTFRIPYYVGPLARGNRDFAWLTR |
| | NSDQAIRPWNFEEVVDKASSAEDFINKMTNYDLYLPEEKVLPKHSLLYETFAVYNELTKVKFIAEG |
| | LRDYQFLDSGQKKQIVNQLFKTKRKVTEKDIINYLHKVDGYDGVEISGVEDRFNASLGTYHDLLKI |
| | IKDKSFLDDKKNEEIIEDIVLTLTLFEDRDMIRQRLQKYSDIFTPQQLKKLERRHYTGWGRLSYKLIN |
| | GIRNKENNKSILDYLIDDGYANRNFMQLINDDTLPFKQIIQKSQVVGDIDDIKTVVHDLPGSPAIKK |
| | GILQSVKIVDELVKVMGHNPDNIVIEMARENQTTNRGRSQSQQRLKKLQESLKELGSNILNVEKPS |
| | YIEDKVENNHLQNDKLFLYYIQNGKDMYTGEELDIDHLSDYDIDHIIPQAFIKDDSIDNRVLTSSAK |
| | NRGKSDDVPSLDIVRGRKAEWIRLYKAKLISQRKFDNLTKAERGGLTENDKAGFIKRQLVETRQIT |
| | KHVAQILDARFNTKRDENDKLIREVKVITLKSNLVSQFRKEFGFYKVREINDYHHAHDAYLNAVV |
| | GTAILKKYPKLTSEFVYGEYKKYDIRKFIKSDDHSEMGKATAKYFFYSNLLNFFKEEIKYANGKIVV |
| | RPVVEYSKDTGEIAWNKEKDFATVRKVLSCPQVNIVKKTEVQTGGFSKESILPKRNSDKLIPRKTE |
| | WDPKKYGGFDSPTVAYSVLVVAKVEKGKSKKLKSVKELVGITIMERSSFEKDPVDFLEAKGYKEV |
| | RKDLIIKLPKYSLFELENGRRRLLASAGELQKGNELALPSKYVNFLYLASHYEKLKGSPEDNEQKQ |
| | LFVEQHKHYLDEIVEQISEFSERVILADANLDKVLSAYNKNRDRPIREQAENIIHLFTLTNLGAPAAF |
| | KFFDTTIDRKRYTSTKEVLDATLIHQSITGLYETRIDLSQLGGD |
| 280 | MKKPYSIGLDIGTNSVGWAIVDDDYKVPAKKMRVFGDTDRSHIKKNLLGTLLFDSGETAESRRLK |
| | RTARRRYTRRRNRILYLQEIFAEEMNKVDDSFFHRLEDSFLVEEDKQGSKYPIFGTLEEEKEYHKQF |
| | PTIYHLRKQLAESNEKADLRLIYLALAHMIKYRGHFLIDDPKFKVQNNDIQGLFEKFVEEYDNVQE |
| | TSLSKIKLNVTEILTANIPKSEKQEQLLKNYPSEKKNTLFGNLIGLALGLTPNFKTNFSLENDAKLQIS |
| | SESYEEDLGNLLALIGENFIELFSAVKNLSDGILLAGIVSDESPHAPLSTKMVIRFKEHEEDLAALKH |
| | FIKANLPEKYDEVFSDDSKNGYAGYVGVDSKVRKRNGKLATEEEFYKYLKDILNNVKGADYFLEK |
| | IKREDFLRKQRTFDNGSIPYQVHLTEMKAILQRQGEHYPFLKENKEKIEKILTFRIPYYVGPLARGNR |
| | DFAWLTRNSDQAIRPWNFEEVVDKASSAEDFINKMTNYDLYLPEEKVLPKHSLLYETFAVYNELT |
| | KVKFIAEGLRDYQFLDSGQKKQIVNQLFKEKRKVTEKDIIHYLHNVDGYDGIELKGIEKQFNASLST |
| | YHDLLKIIKDKEFMDDPKNEEILENIVHTLTIFEDREMIKQRLAQYDSIFDEKVIKALTRRHYTGWG |
| | KLSAKLINGICDKKTGKTILDYLIDDGKINRNFMQLINDDGLSFKEIIQKAQVVGKTDDVKQVVQEL |
| | PGSPAIKKGILQSIKIVDELVKVMGHAPESIVIEMARENQTTARGKKNSQQRYKRIEDALKNLAPGL |
| | DSNILKENPTDNIQLQNDRLFLYYLQNGKDMYTGEPLDINQLSSYDIDHIIPQAFIKDDSLDNRVLTS |
| | SKDNRGKSDNVPSIEVVQKRKAFWQQLLDSKLISERKENNLTKAERGGLDERDKVGFIKRQLVETR |
| | QITKHVAQILDARFNTEVDENNQKIREVKIITLKSNLVSNFRKEFGLYKVREINDYHHAHDAYLNA |
| | VVAKAILKKYPKLEPEFVYGDYQKYDIKRYISRSKDPKDIEKATEKYFFYSNLLNFFKEEVHYADG |
| | TIIKRENIEYSKDTGEIAWNKEKDFATVRKVLSCPQVNIVKKTEVQTGGFSKESILPKRNSDKLIARK |
| | KDWDPKKYGGFDSPTVAYSVLVVAKVEKGKSKKLKSVKELVGITIMERSSFEKDPVDFLEAKGYK |
| | EVRKDLIIKLPKYSLFELENGRKRMLASAGELQKGNELALPSKYVNFLYLASHYEKLKGSPEDNEQ |
| | KQLFVEQHKHYLDEIIEQISEFSKRVILADANLDKVLSAYNKHRDKPIREQAENIIHLFTLTNLGAPA |
| | AFKYFDTTIDRKRYTSTKEVLDATLIHQSITGLYETRIDLSQLGGD |
| 281 | MKKPYSIGLDIGTNSVGWAVVTDDYKVPAKKMKVLGNTDKSHIKKNLLGALLFDSGNTAEDRRL |
| | KRTARRRYTRRRNRILYLQEIFAEEMNKVDESFFHRLDDSFLVPEDKRGSKYPIFGTLEEEKEYHKQ |
| | FPTIYHLRKQLAESNEKADLRLVYLALAHMIKYRGHFLIDDPKFKVQNNDIQGLFEKFVEEYDNVQ |
| | ETSLSKIKLNVTEILTANIPKSEKQEQLLKNYPSEKKNTLFGNLIGLALGLTPNFKTNFSLENDAKLQI |
| | SSESYEEDLGSLLALIGENFIELFSAVKNLSDGILLAGIVSDESPHAPLSTKMVIRFKEHEEDLAALKH |
| | FIKANLPEKYDEVFSDDSKNGYAGYVGVDSKVRKRNGKLATEEEFYKYLKEILNNLKGADYFLEK |
| | IKREDLLRKQRTFDNGTIPYQVHLTEMKAIIHRQGEHYPFLKENKEKIEKILTFRIPYYVGPLARGNR |
| | DFAWLTRNSDQAIRPWNFEEVVDKASSAEDFINKMTNYDLYLPEEKVLPKHSLLYETFAVYNELT |
| | KVKFIAEGLRDYQFLDSGQKKQIVNQLFKEKRKVTEKDIIHYLHNVDGYDGIELKGIEKQFNASLST |
| | YHDLLKIIKDKEFMDDPKNEEILENIVHTLTIFEDREMIKQRLAQYDSLFDEKVIKALTRRHYTGWG |
| | KLSAKLINGICDKQTGKTILDYLIDDDKINRNFMQLINDDGLSFKEIIQKAQVVGKTDDVKQVVQEL |
| | PGSPAIKKGILQSIKIVDELVKVMGHAPESIVIEMARENQTTARGKKNSQQRYKRIEDALKNLAPGL |
| | DSNILKENPTDNIQLQNDRLFLYYLQNGKDMYTGEALDINQLSNYDIDHIIPQAFIKDDSLDNRVLT |
| | SSKDNRGKSDNVPSLEVVEKMKAFWQQLLDSKLISERKYNNLTKAERERGGLNELDKVGFIKRQL |
| | VETRQITKHVAQILDARFNTEVNENNQKIRKVKIITLKSNLVSNFRKEFGLYKVREINDYHHAHDA |
| | YLNAVVAKAILKKYPKLEPEFVYGDYQKYDIKRYISRSKDPKEIEKATEKYFFYSNLLNFFKEEVH |
| | YADGTIVKRENIEYSKDTGEIAWNKEKDFATVRKVLSCPQVNIVKKTEVQTGGFSKESILPKRNSD |
| | KLIPRKTEWDPKKYGGFDSPIVAYSVLVVAKVEKGKSKKLKPVKELVGITIMERSSFEKDPVDFLE |
| | AKGYKEVRKDLIIKLPKYSLFELENGRKRMLASAGELQKGNELALPSKYVNFLYLASHYEKLKGSP |
| | EDNEQKQLFVEQHKHYLDEIVEQISEFSERVILADANLDKILSAYNKHRDKPIREQAENIINLFTFTN |
| | LGAPAAFKFFDTDIDRKRYTSTKEVLDATLIHQSITGLYETRIDLSQLGGD |
| 282 | MNKPYSIGLDIGTNSVGWSVVDDDYKVPAKKMRVFGDTDKGHIKKNLLGTLLFDSGETAESRRLK |
| | RTARRRYTRRRNRILYLQEIFAKEMAKVDESFFHRLDESFLTDDDKTFDSHPIFGNKAEEDAYHQQ |
| | FPTIYHLRKHLADSTEKADLRLVYLALAHMIKFRGHFLIEGELNAENTDVQKLFADFVGVYDRTFD |
| | DSHLSEITVDASSILTEKISKSRRLEKLIKHYPTEKKNTLFGNLVALALGLQPNFKTNFKLSEDAKLQ |
| | FSKDTYEEDLEELLGKIGDDYADLFTSAKNLYDAILLSGILTVDDESTKAPLSASMIKRYAEHHEDL |
| | EKLKEFVKANKPELYHDIFKDESKNGYAGYIENGVKQDEFYKYLKSTLSKQDGSQYFLDKIEREDF |
| | LRKQRTFDNGSIPHQLHLKEMRSILHRQGEYYPFLKENQAKIEKILTFRIPYYVGPLARKDSRFAWA |
| | EYHSDEPITPWNFDEVVDKEKSAEKFITRMTLNDLYLPEEKVLPKHSHVYETYTVYNELTKVKYV |
| | NEQGEAKFFDANMKQEIFDHVFKENRKVTKDKLMDFLGKEFDEFRIVDITGLDKENKAFNASLGT |
| | YHDLKKILDKSFLDDKENETIIEDIIQTLTLFEDRDMIRQRLQKYSDIFTPQQLKKLERRHYTGWGRL |
| | SYKLINGIRDKQTGKTILDYLIDDGYANRNFMQLINDDTLPFKEIIQKAQIIGDIDDIKAVVHDLPGSP |
| | AIKKGILQSVKIVDELVKVMGHNPDNIVIEMARENQTTNRGRTQSQQRLKKLQESLKELGSNILNE |
| | KKPSYVEDKVENTHLQNDRLFLYYIQNGKDMYTGDELDIDHLSDYDIDHIIPQAFIKDDSIDNRVLT |
| | SSAKNRGKSDDVPSIEIVRDRKADWMRLRKAGLISKRKFDNLTKAERGGLTEADKAGFIKRQLVET |
| | RQITKHVAQILDSRENTERDENDKVIREVKVITLKSNLVSQFRKDFQFYKVREINDYHHAHDAYLN |
| | AVVGTALIKKYPKLESEFVYGDYKVYDVRKMIAKSEQEIGKATAKRFFYSNIMNFFKTEIKLANGE |
| | IRKRPLIETNEETGEIVWNKEKDFATVRKVLSCPQVNIVKKTEVQTGGFSKESILPKRNSDKLIPRKK |
| | DWDPKKYGGFDSPTVAYSVLVVAKVEKGKSKKLKPVKELVGITIMERSSFEKNPIDFLEAKGYKE |
| | VQKDLIIKLPKYSLFELENGRRRMLASAGELQKGNELALPSKYVNFLYLASHYEKLKGSPEDNEQK |
| | QLFVDQHKHYLDEIIEQISEFSERVILADANLDKVLSAYNKHRDKPVREQAENIIHLFTLTNLGAPA |
| | AFKFFDTTIDRKRYTSTKEILDATLIHQSITGLYETRIDLSQLGGD |
| 283 | MKKPYSIGLDIGTNSVGWAVVDDDYKVPAKKMRVFGDTDRSHIKKNLLGTLLFDDGNTAESRRL |
| | KRTARRRYTRRRNRILYLQEIFAEEMNKVDESFFHRLDDSFLVPEDKRGSKYPIFATLEEEKEYHKK |
| | FPTIYHLRKHLADSKEKTDLRLIYLALAHMIKYRGHFLYEESFDIKNNDIQKIFNEFISLYDNTFEGSS |
| | LSGQNAQVEAIFTDKISKSAKRERVLKLFPDEKSTGLFSEFLKLIVGNQADFKKHFDLEEKAPLQFS |
| | KDTYDEDLENLLGQIGDDFADLFLVAKKLYDAILLSGILTVTDPSTKAPLSASMIERYENHEEDLAA |
| | LKQFVKANLPEKYDEVFSDQSKDGYAGYIDGKTTQEAFYKYIKNLLSKLEGADYFLDKIEREDFLR |
| | KQRTFDNGSIPHQIHLQEMNAILRRQGEHYPFLKDNKEKIEKILTFRIPYYVGPLARGNRDFAWLTR |
| | NSDQAIRPWNFEEVVDKASSAEDFINKMTNYDLYLPEEKVLPKHSLLYETFAVYNELTKVKFIAEG |
| | LRDYQFLDSGQKKQIVNQLFKTKRKVTEKDIINYLHKVDGYDGVEISGVEDRFNASLGTYHDLLKI |
| | IKDKSFLDDKKNEEIIEDIVLTLTLFEDRDMIRQRLQKYSDIFTPQQLKKLERRHYTGWGRLSYKLIN |
| | GIRNKENNKSILDYLIDDGYANRNFMQLINDDTLPFKQIIQKSQVVGDIDDIKTVVHDLPGSPAIKK |
| | GILQSVKIVDELVKVMGHNPDNIVIEMARENQTTNRGRSQSQQRLKKLQESLKELGSNILNVEKPS |
| | YIEDKVENNHLQNDKLFLYYIQNGKDMYTGEELDIDHLSDYDIDHIIPQAFIKDDSIDNRVLTSSAK |
| | NRGKSDDVPSLDIVRGRKAEWIRLYKAKLISQRKFDNLTKAERGGLTENDKAGFIKRQLVETRQIT |
| | KHVAQILDARFNTKRDENDKLIREVKVITLKSNLVSQFRKEFGFYKVREINDYHHAHDAYLNAVV |
| | GTAILKKYPKLTSEFVYGEYKKYDIRKFIKSDDHSEMGKATAKYFFYSNLLNFFKEEIKYANGKIVV |
| | RPVVEYSKDTGEIAWNKEKDFATVRKVLSCPQVNIVKKTEVQTGGFSKESILPKRNSDKLIARKKD |
| | WDPKKYGGFDSPTVAYSVLVVAKVEKGKSKKLKSVKELVGITIMERSSFEKDPVDFLEAKGYKEV |
| | RKDLIIKLPKYSLFELENGRKRMLASAGELQKGNELALPSKYVNFLYLASHYEKLKGSPEDNEQKQ |
| | LFVEQHKHYLDEIIEQISEFSKRVILADANLDKVLSAYNKHRDKPIREQAENIIHLFTLTNLGAPAAF |
| | KYFDTTIDRKRYTSTKEVLDATLIHQSITGLYETRIDLSQLGGD |
| 284 | MTKPYSIGLDIGTNSVGWAVITDSYKVPSKKMKVLGNTSKKYVKKNLLGALLFESGITAEGRRLK |
| | RTARRRYTRRRNRILYLQEIFSTEMAKIDESFFQRLDDSFLVPDDKRDSKYPIFGNLVKEKTYHDEF |
| | PTIYHLRKYLADSSKKADLRLVYLALAHMIKYRGHFLIEGDFNSRNNDIKKNFQNFLDSYNAIFESD |
| | LSLENSKQLEEIVKDKISKSAKKERVLKLFPDEKSTGLFSEFLKLIVGNQADFKKHFDLEEKAPLQFS |
| | KDTYDEDLENLLGQIGDDFADLFLVAKKLYDAILLSGILTVTDPSTKAPLSASMIERYENHQKDLSA |
| | LKQFIRSNLSEKYDEVFSDQSKDGYAGYINGKTTQEAFYKYIKNLLSKFEGADYFLDKIEREDFLRK |
| | QRTFDNGSIPHQIHLQEMNAIIRRQGEHYPFLKENKEKIEKILTFRIPYYVGPLARGNRDFAWLTRNS |
| | DQAIRPWNFEEVVDKASSAEAFINKMTNFDLYLPEEKVLPKHSLLYETFAVYNELTKVKFIAEGLR |
| | DYQFLDSGQKKQIVNQLFKEKRKVTEKDIIQFLHNVDGYDGIELKGIEKQFNASLATYHDLLKIIKD |
| | KEFMDDPKNEEILENIIHTLTIFEDREMIKQRLAQYDSLFDEKVIKALTRRHYTGWGKLSAKLINGIC |
| | DKQTGKTILDYLIDDGFSNRNFMQLINDDTLPFKQIIQKAQIVGDVDDIEQVVHDLPGSPAIKKGILQ |
| | SIKIVDELVKVMGHNPDNIVIEMARENQTTNRGRSQSQQRLKKLQDSLKELGSNILNEEKPSYIESK |
| | VENSQLQNDRLFLYYIQNGKDMYTGDELDIDRLSDYDIDHIIPQAFIKDDSIDNRVLTSSAKNRGKS |
| | DDVPSLEVVRKRKTYWHQLLKSKLISQRKFDNLTKAERGGLTEKDKAGFIKRQLVETRQITKHVA |
| | QILDSRFNTKRDENDKVIRDVKVITLKSNLVSQFRKDFGFYKVREINDYHHAHDAYLNAVVGTAL |
| | LKKYPKLAPEFVYGEYKKYDVRKIVRSKDESSLGKATAKMFFYSNLMNFFKKEVRLSDGTVIVRP |
| | VIEYSKDTGEIAWNKEKDFATVRKVLSCPQVNIVKKTEVQTGGFSKESILPKRNSDKLIPRKKDWD |
| | PKKYGGFDSPTVAYSVLVVAKVEKGKSKKLKSVKELVGITIMERSSFEKDPVDFLEAKGYKEVRK |
| | DLIIKLPKYSLFELENGRKRMLASAGELQKGNELALPSKYVNFLYLASHYEKLKGSPEDNEQKQLF |
| | VEQHKHYLDEIIEQISEFSERVILADANLDKVLSAYNNHRDKPIREQAENIIHLFTLTNLGAPAAFKF |
| | FDTTIDRKRYTSTKEVLDATLIHQSITGLYETRIDLSKLGGD |
| 285 | MKKSYSIGLDIGTNSVGWAVVDDDYKVPAKKMRVFGDTDRSHIKKNLLGTLLFDEGNTAESRRL |
| | KRTARRRYTRRRNRILYLQEIFATEMNKVDESFFHRLDDSFLVPEDKRGSKYPIFATLEEEKEYHKQ |
| | FPTIYHLRKQLAESNEKADLRLVYLALAHMIKYRGHFLIDDPKFKVQNNDIQGLFEKFVEEYDNVQ |
| | ETSLSKIKLNVTEILTANIPKSEKQEQLLKNYPSEKKNTLFGNLIGLALGLTPNFKTNFSLENDAKLQI |
| | SSESYEEDLGNLLALIGENFIELFSAVKNLSDGILLAGIVSDESPHAPLSAKMVIRFKEHEEDLAALK |
| | HFIKANLPEKYDEVFSDDSKNGYAGYVGVDSKVRKRNGKLATEEEFYKYLKEILNNVKGADYFLE |
| | KIKREDFLRKQRTFDNGTIPYQVHLTEMRAILHRQGEYYPFLKENKEKIEKILTFRIPYYVGPLARG |
| | NRDFAWLTRNSDQAIRPWNFEEVVDKASSAEDFINKMTNYDLYLPEEKVLPKHSLLYETFAVYNE |
| | LTKVKFIAEGLRDYQFLDSGQKKQIVNQLFKEKRKVTEKDIIHYLHNVDGYDGIELKGIEKQFNASL |
| | STYHDLLKIIKDKEFMDDPKNEEILENIVHTLTIFEDREMIKQRLAQYDSLFDEKVIKALTRRHYTG |
| | WGKLSAKLINGIYDKQSKKTILDYLIDDDKINRNFMQLINDDGLSFKEIIQKAQVVGKTDDVKQVV |
| | QELPGSPAIKKGILQSIKIVDELVKVMGHAPESIVIEMARENQTTARGKKNSQQRYKRIEDALKNLA |
| | PGLDSNILKENPTDNIQLQNDRLFLYYLQNGKDMYTGEELDINQLSNYDIDHIVPQAFIKDDSLDNR |
| | VLTSSKDNRGKSDNVPSLEVVQKRKAFWQQLLDSKLISERKENNLTKAERGGLDERDKVGFIRRQ |
| | LVETRQITKHVAQILDARFNTEVTENNQKIRKVKIITLKSNLVSNFRKEFGLYKVREINDYHHAHDA |
| | YLNAVVAKAILKKYPKLEPEFVYGDYQKYDIKRYISRSKDPKEVEKATEKYFFYSNLLNFFKEEVH |
| | YADGTIVKRENIEYSKDTGEIAWNKEKDFATVRKVLSCPQVNIVKKTEVQTGGFSKESILPKRNSD |
| | KLIPRKTEWDPKKYGGFDSPIVAYSVLVVAKVEKGKSKKLKPVKELVGITIMERSSFEKDPVDFLE |
| | AKGYKEVRKDLIIKLPKYSLFELENGRKRMLASAGELQKGNELALPSKYVNFLYLASHYEKLKGSP |
| | EDNEQKQLFVEQHKHYLDEIVEQISEFSERVILADANLDKILSAYNKHRDKPIREQAENIINLFTFTN |
| | LGAPAAFKFFDTDIDRKRYTSTKEVLDATLIHQSITGLYETRIDLSQLGGD |
| 286 | MDKKYSIGLDIGTNSVGWAVITDDYKVPAKKMKVLGNTDKRTIKKNLLGALLFDSGETAEATRLK |
| | RTARRRYTRRKNRLRYLQEIFSKEMSNVDANFFYRLEDSFLVPEDKQQERHPIFGNLVEEIEYHKEF |
| | PTIYHLRKQLADSKEKADLRLIYLALAHMIKYRGHELFEGDLKSENTDVQRLFKDFVEVYDKTVEG |
| | SYLSENLTNVTDVLVEKISKSRRLENILHYFPNEKKNGLFGNFLALALGLQPNFKNNFDLAEEAKL |
| | QISKDTYEEELEVLLAQIGDNYAELFIATKELYDGILLAGILTVTDSSTKAPLSASMIERYENHQGDL |
| | AKLKAFIRRFCKETYDEVFSDEVKDGYAGYIEGKTSQEDFYVHLKKLLANLEGADYFLEKINREDL |
| | LRKQRTFDNGSIPYQIHLVEMRAILDKQAKFYPFLAKNQDRIEKLLTFRIPYYVGPLARGKSDFAWL |
| | SRKSADKITPWNFDEIVDKESSAEAFINRMTNYDLYLPNQKVLPKHSLLYEKFTVYNELTKVKYKT |
| | EQGKTAFFDANMKQEIFDGVFKVYRKVTKDKLMDFLEKEFDEFRIVDLMGLDKENKAFNASYGT |
| | YHDLRKILDKDFLDNSDNEKILEDIVLTLTLFEDREMIRKRLEKYKDIFTEKQRKQLERRHYTGWG |
| | RLSAKLIHGIRNKESRKTILDYLIDDGYSNRNFMQLINDDTLTFKEEIAKAQVVGETDSLKQFISELA |
| | GSPAIKKGILQTVKIVDELVKVMGHKPENIVIEMARENQTTQKGQKNSQERMKRIEEGIKELGSQIL |
| | KEHPVENTQLQNEKLYLYYLQNGRDMYVDQELDINRLSDYDIDHIIPQAFIKDDSIDNRVLTSSKD |
| | NRGKSDNVPSEDVVKKMKNYWRQLLNAKLISQRKFDNLTKAERGGLSELDKVGFIKRQLVETRQI |
| | TKHVAQILDSRMNTKYDENDKLIRDVKIITLKSKLVSDFRKDFQFYKVREINNYHHAHDAYLNAV |
| | VGTALIKKYPKLESEFVYGDYKVYDVQKMIAKSEQEIGKATAKFFFYSNIMNFFKTEITLANGEIRK |
| | RPMIETNEETGEIVWDKGRDFATVRKVLCMPQVNIVKKTEVQTGGFSKESILPKRNSDKLIPRKKG |
| | WDPKKYGGFDSPTVAYSVLVVAKVEKGKSKKLKSVKELVGITIMERSSFEKDPVDFLEAKGYKEV |
| | RKDLIIKLPKYSLFELENGRKRMLASAGELQKGNELALPSKYVNFLYLASHYEKLKGSPEDNEQKQ |
| | LFVEQHKHYLDEIVEQISEFSERVILADANLDKVLSAYNKNRDRPIREQAENIIHLFTLTNLGAPAAF |
| | KFFNTDIDRKRYTSTKEILDATLIHQSITGLYETRIDLSQLGGD |
| 287 | MKKPYSIGLDIGTNSVGWAVVTDDYKVPAKKMKVLGNTNKESIKKNLIGALLFDAGNTAADRRL |
| | KRTARRRYTRRRNRILYLQEIFAEEMNKVDESFFHRLDDSFLVPEDKRGSKYPIFATLEEEKEYHKK |
| | FPTIYHLRKHLADSKEKADLRLIYLALAHMIKYRGHFLYEESFDIKNNDIQKIFNEFISIYDNTFEGSS |
| | LSGQNAQVEAIFTDKISKSAKRERVLKLFPDEKSTGLFSEFLKLIVGNQADFKKHFDLEEKAPLQFS |
| | KDTYDEDLENLLGQIGDDFADLFLVAKKLYDAILLSGILTVTDPSTKAPLSASMIERYENHQKDLA |
| | ALKQFIQNNLQEKYDEVFSDQSKDGYAGYIDGKTTQEAFYKYIKNLLSKLEGADYFLDKIEREDFL |
| | RKQRTFDNGSIPHQIHLQEMNAIIRRQGEHYPFLKENKEKIEKILTFRIPYYVGPLARGNRDFAWLTR |
| | NSDQAIRPWNFEEIVDQASSAEDFINKMTNYDLYLPEEKVLPKHSLLYETFAVYNELTKVKFIAEG |
| | MRDYQFLDSGQKKKIVSQLFEDKRKVTEKDIIHYLHTIDGYDGIELSGIEKQFNASLSTYHDLLKIIK |
| | DKAFLDDSENEAIIEEIIHTLTIFEDREMIKQRLAQYADIFDKSVLKKLARRHYTGWGKLSAKLINGI |
| | RDKQSNKTILDYLIDDGKINRNFMQLINDDSLSFKETIQKAQVVGDTDDVKQVVQELPGSPAIKKGI |
| | LQSIKIVDELVKVMGHNPDNIVIEMARENQTTNRGRSQSQQRLKKLQDSLKELGSNILNEEKPSYIE |
| | DKVENSHLQNDQLFLYYIQNGKDMYTGEELDIDHLSDYDIDHIIPQAFIKDDSIDNRVLTSSAKNRG |
| | KSDDVPSLDIVRERKADWIRLYKSKLISKRKFDNLTKAERGGLTEADKAGFIKRQLVETRQITKHV |
| | AQILDSRFNTKRDENDKVIRDVKVITLKSNLVSQFRKDFELYKVREINDYHHAHDAYLNAVVGTA |
| | LLKKYPKLAPEFVYGEYKKYDIRKFITSSKDKATAKYFFYSNLMNFFKRIIRYSNGKIVVRPVVEYS |
| | KDTGEIAWNKEKDFATVRKVLSCPQVNIVKKTEVQTGGFSKESILPKRNSDKLIARKKDWDPKKY |
| | GGFDSPTVAYSVLVVAKVEKGKSKKLKSVKELVGITIMERSSFEKDPVDFLEAKGYKEVRKDLIIK |
| | LPKYSLFELENGRKRMLASAGELQKGNELALPSKYVNFLYLASHYEKLKGSPEDNEQKQLFVEQH |
| | KHYLDEIIEQISEFSKRVILADANLDKVLSAYNKHRDKPIREQAENIIHLFTLTNLGAPAAFKYFDTTI |
| | DRKRYTSTKEVLDATLIHQSITGLYETRIDLSQLGGD |
| 288 | MKKSYSIGLDIGTNSVGWSVITDDYKVPAKKMKVLGNTDKKYIKKNLLGALLFDSGETAEATRLK |
| | RTARRRYTRRKNRLRYLQEIFAKEMAKVDESFFQRLEESFLTDDDKNFDSHPIFGNKAEEDAYHDE |
| | FPTIYHLRQYLADSPQKADLRLVYLALAHIIKFRGHFLIEGQLNAENTDVQTLFNDFVEVYDKTVEE |
| | SHLSEITVDALSILTEKVSKSRRLENLVKCYPTEKKNTLFGNLIALSLGLQPNFKTNFKLSEDAKLQF |
| | SKDTYEEDLEELLGKIGDEYADLFASAKNLYDAILLSGILTADDNSTKAPLSASMVKRYAEHHEDL |
| | EKLKEFVKANKPELYHDIFKDETKNGYAGYIENGVKQDEFYKYLKNTLSKIAGSDYFLDKIEREDF |
| | LRKQRTFDNGSIPHQIHLQEMHAILRRQGEYYPFLKENQAKIEKILTFRIPYYVGPLARKNSRFAWA |
| | EYHSDEKITPWNFEEIVDKEESATAFIHRMTNNDLYLPEEKVLPKHSLIYEEFTVYNELTKVRYVTD |
| | QGKAYYFDANMKQEIFDGVFKEHRKVTKDKLMNFLEKEFDEFRIVDLTGLDKENKSFNASLGTYH |
| | DLKKILDKSFLDDKTNETIIEDIIQTLTLFEDRDMIRQRLQKYSDIFTPQQLKKLERRHYTGWGRLSY |
| | KLINGIRNKENGKTILDYLIDDGYANRNFMQLINDDTLPFKQIIQEAQIIGDIDDIETVVHDLPGSPAI |
| | KKGILQSVKIVDELVKVMGHNPDNIVIEMARENQTTNRGRSQSQQRLKKLQESLKELGSNILNEEK |
| | PSYVEDKVENSHLQNDKLFLYYIQNGKDMYTGEELDIDHLSDYDIDHIIPQAFIKDNSIDNRVLTSS |
| | AKNRGKSDDVPSLDIVHDRKADWIRLYKSGLISKRKFDNLTKAEHGGLTEDDKAGFIKRQLVETR |
| | QITKHVAQILDSRENTERDENDKVIRDVKVITLKSNLVSQFRKDFELHKVREINDYHHAHDAYLNA |
| | VIGNALLVKYPQLEPEFVYGKFSHFHGHKENKATAKKFFYSNIMALFKKDVEYSDDTGEMVWNK |
| | EKDFATVRKVLSCPQVNIVKKTEVQTGGFSKESILPKRNSDKLIPRKKDWDPKKYGGFDSPIVAYS |
| | VLVVAKVEKGKSKKLKPVKELVGITIMERNSFEKDPIDFLEAKGYKEVRKDLIIKLPKYSLFELENG |
| | RKRMLASAGELQKGNELALPSKYVNFLYLASHYEKLKGSPEDNEQKQLFVEQHKHYLDEIIEQISE |
| | FSERVILADANLDKVLSAYNNHRDKPIREQAENIIHLFTLTNLGAPAAFKFFDTTIDRKRYTSTKEVL |
| | DATLIHQSITGLYETRIDLSQLGGD |
| 289 | MKKSYSIGLDIGTNSVGWSVITDDYKVPAKKMKVLGNTDKKYIKKNLLGALLFDSGETAEATRLK |
| | RTARRRYTRRKNRLRYLQEIFAKEMAKVDESFFQRLEESFLTDDDKNFDSHPIFGNKAEEDAYHDE |
| | FPTIYHLRQYLADSPQKADLRLVYLALAHMIKFRGHFLIEGQLNAENTDVQTLFNDFVEVYDKTVE |
| | ESHLSEITVDALSILTEKVSKSRRLENLVKCYPTEKKNTLFGNLIALSLGLQPNFKTNFKLSEDAKLQ |
| | FSKDTYEEDLEELLGKIGDEYADLFASAKNLYDAILLSGILTVDDNTTKAPLSASMVKRYAEHHED |
| | LEKLKEFVKANKPELYHDIFKDETKNGYAGYIENGVKQDEFYKYLKNTLSKIAGSDYFLDKIERED |
| | FLRKQRTFDNGSIPHQIHLQEMHAILRRQGEYYPFLKENQAKIEKILTFRIPYYVGPLARKNSRFAW |
| | AEYHSDEKITPWNFEEIVDKEESATAFIHRMTNNDLYLPEEKVLPKHSLIYEEFTVYNELTKVKYVT |
| | DQGKAYYFDANMKQEIFDGVFKEHRKVTKDKLMNFLEKEFDEFRIVDLTGLDKENKSFNASLGTY |
| | HDLKKILDKSFLDDKTNETIIEDIIQTLTLFEDRDMIRQRLQKYSDIFTPQQLKKLERRHYTGWGRLS |
| | YKLINGIRNKENGKTILDYLIDDGYANRNFMQLINDDTLPFKQIIQEAQIIGDIDDIETVVHDLPGSPA |
| | IKKGILQSVKIVDELVKVMGHNPDNIVIEMARENQTTNRGRSQSQQRLKKLQESLKELGSNILNEEK |
| | PSYVEDKVENSHLQNDKLFLYYIQNGKDMYTGEELDIDHLSDYDIDHIIPQAFIKDNSIDNRVLTSS |
| | AKNRGKSDDVPSLDIVHDRKADWIRLYKSGLISKRKFDNLTKAEHGGLTEDDKAGFIKRQLVETR |
| | QITKHVAQILDSRENTERDENDKVIRDVKVITLKSNLVSQFRKDFELHKVREINDYHHAHDAYLNA |
| | VIGNALLVKYPQLEPEFVYGKFKHFHGHKENKATAKKFFYSNIMALFKKDVEYSDDTGEMVWNK |
| | EKDFATVRKVLSCPQVNIVKKTEVQTGGFSKESILPKRNSDKLIARKKDWDPKKYGGFDSPTVAYS |
| | VLVVAKVEKGKSKKLKSVKELVGITIMERSSFEKDPIDFLEAKGYKEVRKDLIIKLPKYSLFELENG |
| | RKRMLASAGELQKGNELALPSKYVNFLYLASHYEKLKGSPEDNEQKQLFVEQHQHYLDEIIEQISE |
| | FSKRVILADANLDKVLSAYNKHRDKPIREQAENIIHLFTLTNLGAPAAFKYFDTTIDRKRYTSTKEV |
| | LDATLIHQSITGLYETRIDLSQLGGD |
| 290 | MTKPYSIGLDIGTNSVGWAVITDSYKVPSKKMKVLGNTSKKYVKKNLLGALLFESGITAEGRRLK |
| | RTARRRYTRRRNRILYLQEIFSTEMAKIDESFFQRLDDSFLVPDDKRDSKYPIFGNLVKEKTYHDEF |
| | PTIYHLRKYLADSSKKADLRLVYLALAHMIKYRGHFLIEGDFNSRNNDIKKNFQNFLDSYNAIFESD |
| | LSLENSKQLEEIVKDKISKSAKKERVLKLFPDEKSTGLFSEFLKLIVGNQADFKKHFDLEEKAPLQFS |
| | KDTYDEDLENLLGQIGDDFADLFLVAKKLYDAILLSGILTVTDPSTKAPLSASMIERYENHQKDLSA |
| | LKQFIRSNLSEKYDEVFSDQSKDGYAGYINGKTTQEAFYKYIKNLLSKFEGADYFLDKIEREDFLRK |
| | QRTFDNGSIPHQIHLQEMNAIIRRQGEHYPFLKENKEKIEKILTFRIPYYVGPLARGNRDFAWLTRNS |
| | DQAIRPWNFEEVVDKASSAEAFINKMTNFDLYLPEEKVLPKHSLLYETFAVYNELTKVKFIAEGLR |
| | DYQFLDSGQKKQIVNQLFKEKRKVTEKDIIQFLHNVDGYDGIELKGIEKQFNASLATYHDLLKIIKD |
| | KEFMDDPKNEEILENIIHTLTIFEDREMIKQRLAQYDSLFDEKVIKALTRRHYTGWGKLSAKLINGIC |
| | DKQTGKTILDYLIDDGFSNRNFMQLINDDTLPFKQIIQKAQIVGDVDDIEQVVHDLPGSPAIKKGILQ |
| | SIKIVDELVKVMGHNPDNIVIEMARENQTTNRGRSQSQQRLKKLQDSLKELGSNILNEEKPSYVESK |
| | VENSQLQNDRLFLYYIQNGKDMYTGEELDIDRLSDYDIDHIIPQAFIKDDSIDNRVLTSSAKNRGKS |
| | DDVPSLEVVRKRKTYWHQLLKSKLISQRKFDNLTKAERGGLTEKDKAGFIKRQLVETRQITKHVA |
| | QILDSRFNTKRDENDKVIRDVKVITLKSNLVSQFRKDFGFYKVREINDYHHAHDAYLNAVVGTAL |
| | LKKYPKLAPEFVYGEYKKYDVRKLVRSKDESSLGKATAKMFFYSNLMNFFKKEVRLSDGTVIVRP |
| | VIEYSKDTGEIAWNKEKDFATVRKVLSCPQVNIVKKTEVQTGGFSKESILPKRNSDKLIPRKKDWD |
| | PKKYGGFDSPTVAYSVLVVAKVEKGKSKKLKPVKELVGITIMERSSFEKDPVDFLEAKGYKEVRK |
| | DLIIKLPKYSLFELENGRKRMLASAGELQKGNELALPSKYVNFLYLASHYEKLKGSPEDNEQKQLF |
| | VEQHKHYLDEIVEQISEFSERVILADANLDKVLSAYNNHRDKPIREQAENIIHLFTLTNLGAPAAFKF |
| | FDTTIDRKRYTSTKEVLDATLIHQSITGLYETRIDLSKLGGD |
| 291 | MTKPYSIGLDIGTNSVGWAVITDSYKVPSKKMKVLGNTSKKYVKKNLLGALLFESGITAEGRRLK |
| | RTARRRYTRRRNRILYLQEIFSTEMAKIDESFFQRLDDSFLVPDDKRDSKYPIFGNLVKEKTYHDEF |
| | PTIYHLRKYLADSSKKADLRLVYLALAHMIKYRGHFLIEGDFNSRNNDIKKNFQNFLDSYNAIFESD |
| | LSLENSKQLEEIVKDKISKSAKRERVLKLFPDEKSTGLFSEFLKLIVGNQADFKKHFDLEEKAPLQFS |
| | KDTYDEDLENLLGQIGDDFADLFLVAKKLYDAILLSGILTATDPSTKAPLSASMIERYENHQKDLST |
| | LKQFIRSNLSEKYDEVFSDQSKDGYAGYINGKTTQEAFYKYIKNLLSKFEGADYFLDKIEREDFLRK |
| | QRTFDNGSIPHQIHLQEMNAIIRRQGEHYPFLKENKEKIEKILTFRIPYYVGPLARGNRDFAWLTRNS |
| | DQAIRPWNFEEVVDKASSAEAFINKMTNFDLYLPEEKVLPKHSLLYETFAVYNELTKVKFIAEGLR |
| | DYQFLDSGQKKQIVNQLFKEKRKVTEKDIIQFLHNVDGYDGIELKGIEKQFNASLATYHDLLKIIKD |
| | KEFMDDPKNEEILENIIHTLTIFEDREMIKQRLAQYDSLFDEKIIKALTRRHYTGWGKLSAKLINGIC |
| | DKQTGKTILDYLIDDGFSNRNFMQLINDDTLPFKQIIQKAQIVGDVDDIEQVVHNLPGSPAIKKGILQ |
| | SIKIVDELVKVMGHNPDNIVIEMARENQTTNRGRSQSQQRLKKLQESLKELGSNILNEEKPSYVESK |
| | VENSQLQNDRLFLYYIQNGKDMYTGEELDIDRLSDYDIDHIIPQAFIKDDSIDNRVLTSSAKNRGKS |
| | DDVPSLEVVKKRKTYWHQLLKSKLISQRKFDNLTKAERGGLTEKDKAGFIKRQLVETRQITKHVA |
| | QILDSRFNTKRDENDKVIRDVKVITLKSNLVSQFRKEFGFYKVREINDYHHAHDAYLNAVVGTALL |
| | KKYPKLAPEFVYGEYKKYDVRKIVRSKDESSLGKATAKMFFYSNLMNFFKKEVRLSDGTVIVRPVI |
| | EYSKDTGEIAWNKEKDFATVKKVLSCPQVNIVKKTEVQTGGFSKESILPKRNSDKLIPRKKDWDPK |
| | KYGGFDSPTVAYSVLVVAKVEKGKSKKLKPVKELVGITIMERSSFEKDPIDFLEAKGYKEVRKDLII |
| | KLPKYSLFELENGRKRLLASAGELQKGNELALPSKYVNFLYLASHYEKLKGSPEDNEQKQLFVEQ |
| | HKHYLDEIVEQISEFSERVILADANLDKVLSAYNKHRDKPIREQAENIIHLFTLTNLGAPAAFKFFDT |
| | TIDRKRYTSTKEVLDATLIHQSITGLYETRIDLSKLGGD |
| 292 | MTKPYSIGLDIGTNSVGWAVITDSYKVPSKKMKVLGNTSKKYVKKNLLGALLFESGITAEGRRLK |
| | RTARRRYTRRRNRILYLQEIFSTEMAKIDESFFQRLDDSFLVPDDKRDSKYPIFGNLVKEKTYHDEF |
| | PTIYHLRKYLADSSKKADLRLVYLALAHMIKYRGHFLIEGDFNSRNNDIKKNFQNFLDSYNAIFESD |
| | LSLENSKQLEEIVKDKISKSAKRERVLKLFPDEKSTGLFSEFLKLIVGNQADFKKHFDLEEKAPLQFS |
| | KDTYDEDLENLLGQIGDDFADLFLVAKKLYDAILLSGILTVTDPSTKAPLSASMIERYENHQKDLSA |
| | LKQFIRSNLSEKYDEVFSDQSKDGYAGYINGKTTQEAFYKYIKNLLSKFEGADYFLDKIEREDFLRK |
| | QRTFDNGSIPHQIHLQEMNAIIRRQGEHYPFLKENKEKIEKILTFRIPYYVGPLARGNRDFAWLTRNS |
| | DQAIRPWNFEEVVDKASSAEAFINKMTNFDLYLPEEKVLPKHSLLYETFAVYNELTKVKFIAEGLR |
| | DYQFLDSGQKKQIVNQLFKEKRKVTEKDIIQFLHNVDGYDGIELKGIEKQFNASLATYHDLLKIIKD |
| | KEFMDDPKNEEILENIIHTLTIFEDREMIKQRLAQYDSLFDEKVIKALTRRHYTGWGKLSAKLINGIC |
| | DKQTGKTILDYLIDDGFANRNFMQLINDDTLPFKQIIQKAQIVGDVDDIEQVVHNLPGSPAIKKGIL |
| | QSIKIVDELVKVMGHNPDNIVIEMARENQTTNRGRSQSQQRLKKLQESLKELGSNILNEEKPSYVES |
| | KVENSQLQNDRLFLYYIQNGKDMYTGEELDIDRLSDYDIDHIIPQAFIKDDSIDNRVLTSSAKNRGK |
| | SDDVPSLEVVKKRKTYWHQLLKAKLISQRKFDNLTKAERGGLTEKDKAGFIKRQLVETRQITKHV |
| | AQILDSRFNTKRDENDKVIRDVKVITLKSNLVSQFRKDFGFYKVREINDYHHAHDAYLNAVVGTA |
| | LLKKYPKLAPEFVYGEYKKYDVRKIVRSKDESSLGKATAKMFFYSNLMNFFKKEVRLSDGTVIVR |
| | PVIEYSKDTGEIAWNKEKDFATVRKVLSCPQVNIVKKTEVQTGGFSKESILPKRNSDKLIARKKDW |
| | DPKKYGGFDSPTVAYSVLVVAKVEKGKSKKLKSVKELLGITIMERSSFEKNPIDFLEAKGYKEVRK |
| | DLIIKLPKYSLFELENGRKRMLASAGELQKGNELALPSKYVNFLYLASHYEKLKGSPEDNEQKQLF |
| | VEQHKHYLDEIIEQISEFSKRVILADANLDKVLSAYNKHRDKPIREQAENIIHLFTLTNLGAPAAFKY |
| | FDTTIDRKRYTSTKEVLDSTLIHQSITGLYETRIDLSQLGGD |
| 293 | MKKSYSIGLDIGTNSVGWAVVDDDYKVPAKKMRVFGDTDRSHIKKNLLGTLLFDEGNTAESRRL |
| | KRTARRRYTRRRNRILYLQEIFATEMNKVDESFFHRLDDSFLVPEDKRGSKYPIFATLEEEKEYHKQ |
| | FPTIYHLRKQLAESNEKADLRLVYLALAHMIKYRGHFLIDDPKFKVQNNDIQGLFEKFVEEYDNVQ |
| | ETSLSKIKLNVTEILTANIPKSEKQEQLLKNYPSEKKNTLFGNLIGLALGLTPNFKTNFSLENDAKLQI |
| | SSESYEEDLGNLLALIGENFIELFSAVKNLSDGILLAGIVSDESPHAPLSAKMVIRFKEHEEDLAALK |
| | HFIKANLPEKYDEVFSDDSKNGYAGYVGVDSKVRKRNGKLATEEEFYKYLKEILNNVKGADYFLE |
| | KIKREDFLRKQRTFDNGTIPYQVHLTEMRAILHRQGEYYPFLKENKEKIEKILTFRIPYYVGPLARG |
| | NRDFAWLTRNSDQAIRPWNFEEVVDKASSAEDFINKMTNYDLYLPEEKVLPKHSLLYETFAVYNE |
| | LTKVKFIAEGLRDYQFLDSGQKKQIVNQLFKEKRKVTEKDIIHYLHNVDGYDGIELKGIEKQFNASL |
| | STYHDLLKIIKDKEFMDDPKNEEILENIVHTLTIFEDREMIKQRLAQYDSLFDEKVIKALTRRHYTG |
| | WGKLSAKLINGIYDKQSKKTILDYLIDDDKINRNFMQLINDDGLSFKEIIQKAQVVGKTDDVKQVV |
| | QELPGSPAIKKGILQSIKIVDELVKVMGHAPESIVIEMARENQTTARGKKNSQQRYKRIEDALKNLA |
| | PGLDSNILKENPTDNIQLQNDRLFLYYLQNGKDMYTGEELDINQLSNYDIDHIVPQAFIKDDSLDNR |
| | VLTSSKDNRGKSDNVPSLEVVQKRKAFWQQLLDSKLISERKENNLTKAERGGLDERDKVGFIRRQ |
| | LVETRQITKHVAQILDARFNTEVTENNQKIRKVKIITLKSNLVSNFRKEFGLYKVREINDYHHAHDA |
| | YLNAVVAKAILKKYPKLEPEFVYGDYQKYDIKRYISRSKDPKEVEKATEKYFFYSNLLNFFKEEVH |
| | YADGTIVKRENIEYSKDTGEIAWNKEKDFATVRKVLSCPQVNIVKKTEVQTGGFSKESILPKRNSD |
| | KLIPRKTEWDPKKYGGFDSPTVAYSVLVVAKVEKGKSKKLKSVKELVGITIMERSSFEKDPVDFLE |
| | AKGYKEVRKDLIIKLPKYSLFELENGRRRLLASAGELQKGNELALPSKYVNFLYLASHYEKLKGSP |
| | EDNEQKQLFVEQHKHYLDEIVEQISEFSERVILADANLDKVLSAYNKNRDRPIREQAENIIHLFTLTN |
| | LGAPAAFKFFDTTIDRKRYTSTKEVLDATLIHQSITGLYETRIDLSQLGGD |
| 294 | MKKPYSIGLDIGTNSVGWAVVTDDYKVPAKKMKVLGNTNKESIKKNLIGALLFDAGNTAADRRL |
| | KRTARRRYTRRRNRILYLQEIFAEEMNKVDESFFHRLDDSFLVPEDKRGSKYPIFATLEEEKEYHKK |
| | FPTIYHLRKHLADSKEKADLRLIYLALAHMIKYRGHFLYEESFDIKNNDIQKIFNEFISIYDNTFEGSS |
| | LSGQNAQVEAIFTDKISKSAKRERVLKLFPDEKSTGLFSEFLKLIVGNQADFKKHFDLEEKAPLQFS |
| | KDTYDEDLENLLGQIGDDFADLFLVAKKLYDAILLSGILTVTDPSTKAPLSASMIERYENHQKDLA |
| | ALKQFIQNNLQEKYDEVFSDQSKDGYAGYIDGKTTQEAFYKYIKNLLSKLEGADYFLDKIEREDFL |
| | RKQRTFDNGSIPHQIHLQEMNAIIRRQGEHYPFLKENKEKIEKILTFRIPYYVGPLARGNRDFAWLTR |
| | NSDQAIRPWNFEEIVDQASSAEDFINKMTNYDLYLPEEKVLPKHSLLYETFAVYNELTKVKFIAEG |
| | MRDYQFLDSGQKKKIVSQLFEDKRKVTEKDIIHYLHTIDGYDGIELSGIEKQFNASLSTYHDLLKIIK |
| | DKAFLDDSENEAIIEEIIHTLTIFEDREMIKQRLAQYADIFDKSVLKKLARRHYTGWGKLSAKLINGI |
| | RDKQSNKTILDYLIDDGKINRNFMQLINDDSLSFKETIQKAQVVGDTDDVKQVVQELPGSPAIKKGI |
| | LQSIKIVDELVKVMGHNPDNIVIEMARENQTTNRGRSQSQQRLKKLQDSLKELGSNILNEEKPSYIE |
| | DKVENSHLQNDQLFLYYIQNGKDMYTGEELDIDHLSDYDIDHIIPQAFIKDDSIDNRVLTSSAKNRG |
| | KSDDVPSLDIVRERKADWIRLYKSKLISKRKFDNLTKAERGGLTEADKAGFIKRQLVETRQITKHV |
| | AQILDSRFNTKRDENDKVIRDVKVITLKSNLVSQFRKDFELYKVREINDYHHAHDAYLNAVVGTA |
| | LLKKYPKLAPEFVYGEYKKYDIRKFITSSKDKATAKYFFYSNLMNFFKRIIRYSNGKIVVRPVVEYS |
| | KDTGEIAWNKEKDFATVRKVLSCPQVNIVKKTEVQTGGFSKESILPKRNSDKLIARKKDWDPKKY |
| | GGFDSPTVAYSVLVVAKVEKGKSKKLKSVKELVGITIMERSSFEKDPIDFLEAKGYKEVRKDLIIKL |
| | PKYSLFELENGRKRMLASAGELQKGNELALPSKYVNFLYLASHYEKLKGSPEDNEQKQLFVEQHK |
| | HYLDEIIEQISEFSKRVILADANLDKVLSAYNKHRDKPIREQAENIIHLFTLTNLGAPAAFKYFDTTID |
| | RKRYTSTKEVLDATLIHQSITGLYETRIDLSQLGGD |
| 295 | MKKSYSIGLDIGTNSVGWSVITDDYKVPAKKMKVLGNTDKKYIKKNLLGALLFDSGETAEGTRLK |
| | RTARRRYTRRKNRLRYLQEIFAKEMAKVDESFFQRLEESFLTDDDKNFDSHPIFGNKAEEDAYHDE |
| | FPTIYHLRQYLADSPQKADLRLVYLALAHMIKFRGHFLIQGQLNAENTDVQTLFNDFVEVYDKTVE |
| | ESHLSEITVDALSILTEKVSKSRRLENLVKCYPTEKKNTLFGNLIALSLGLQPNFKTNFKLSEDAKLQ |
| | FSKDTYEEDLEELLGKIGDEYADLFASAKNLYDAILLSGILTVDDNSTKAPLSASMVKRYAEHHED |
| | LEKLKEFVKANKPELYHDIFKDETKNGYAGYIENGVKQDEFYKYLKNTLSKIAGSDYFLDKIERED |
| | FLRKQRTFDNGSIPHQIHLQEMHAILRRQGEYYPFLKENQAKIEKILTFRIPYYVGPLARKNSRFAW |
| | AEYHSDEKITPWNFEEIIDKEESATAFIHRMTNNDLYLPEEKVLPKHSLIYEEFTVYNELTKVRYVT |
| | DQGKAYYFDANMKQEIFDGVFKEHRKVTKDKLMNFLEKEFDEFRIVDLTGLDKENKSFNASLGTY |
| | HDLKKILDKSFLDDKTNETIIEDIIQTLTLFEDRDMIRQRLQKYSDIFTPQQLKKLERRHYTGWGRLS |
| | YKLINGIRNKENGKTILDYLIDDGYANRNFMQLINDDTLPFKQIIQEAQIIGDIDDIETVVHDLPGSPA |
| | IKKGILQSVKIVDELVKVMGHNPDNIVIEMARENQTTNRGRSQSQQRLKKLQESLKELGSNILNEEK |
| | PSYIEDKVENSHLQNDRLFLYYIQNGKDMYTGEELDIDHLSDYDIDHIIPQAFIKDNSIDNRVLTSSA |
| | KNRGKSDDVPSLDIVHDRKADWIRLYKSGLISKRKFDNLTKAEHGGLTEDDKAGFIKRQLVETRQI |
| | TKHVAQILDSRENTERDENDKVIRDVKVITLKSNLVSQFRKDFELHKVREINDYHHAHDAYLNAVI |
| | GNALLVKYPQLEPEFVYGKFSHFHGHKENKATAKKFFYSNIMALFKKDVEYSDDTGEMVWNKEK |
| | DFATVRKVLSCPQVNVVKKTEVQTGGFSKESILPKRNSDKLIPRKNDWDPKKYGGFDSPIVAYSVL |
| | VIAKVEKGKSKKLKPVKELVGITIMERSSFEKDPIDFLEAKGYKDVRKDLIIKLPKYSLFELENGRKR |
| | MLASAGELQKGNELALPSKYVNFLYLASHYEKLKGSPEDNEQKQLFVEQHKHYLDEIIEQISEFSER |
| | VILADSNLDKVLSAYNNHRDKPIREQAENIIHLFTLTNLGAPAAFKFFDTTIDRKRYTSTKEILDATLI |
| | HQSITGLYETRIDLSQLGGD |
| 296 | MTKPYSIGLDIGTNSVGWAVITDSYKVPSKKMKVLGNTSKKYVKKNLLGALLFESGITAEGRRLK |
| | RTARRRYTRRRNRILYLQEIFSTEMAKIDESFFQRLDDSFLVPDDKRDSKYPIFGNLVKEKTYHDEF |
| | PTIYHLRKYLADSSKKADLRLVYLALAHMIKYRGHFLIEGDFNSRNNDIKKNFQNFLDSYNAIFESD |
| | LSLENSKQLEEIVKDKISKSAKKERVLKLFPDEKSTGLFSEFLKLIVGNQADFKKHFDLEEKAPLQFS |
| | KDTYDEDLENLLGQIGDDFADLFLVAKKLYDAILLSGILTVTDPSTKAPLSASMIERYENHQKDLSA |
| | LKQFIRSNLSEKYDEVFSDQSKDGYAGYINGKTTQEAFYKYIKNLLSKFEGADYFLDKIEREDFLRK |
| | QRTFDNGSIPHQIHLQEMNAIIRRQGEHYPFLKENKEKIEKILTFRIPYYVGPLARGNRDFAWLTRNS |
| | DQAIRPWNFEEVVDKASSAEAFINKMTNFDLYLPEEKVLPKHSLLYETFAVYNELTKVKFIAEGLR |
| | DYQFLDSGQKKQIVNQLFKEKRKVTEKDIIQFLHNVDGYDGIELKGIEKQFNASLATYHDLLKIIKD |
| | KEFMDDPKNEEILENIIHTLTIFEDREMIKQRLAQYDSLFDEKVIKALTRRHYTGWGKLSAKLINGIC |
| | DKQTGKTILDYLIDDGFSNRNFMQLINDDTLPFKQIIQKAQIVGDVDDIEQVVHDLPGSPAIKKGILQ |
| | SIKIVDELVKVMGHNPDNIVIEMARENQTTNRGRSQSQQRLKKLQDSLKELGSNILNEEKPSYIESK |
| | VENSQLQNDRLFLYYIQNGKDMYTGDELDIDRLSDYDIDHIIPQAFIKDDSIDNRVLTSSAKNRGKS |
| | DDVPSLEVVKKRKTFWHQLLKSKLISQRKFDNLTKAERGGLTEKDKAGFIKRQLVETRQITKHVA |
| | QILDSRFNTKRDENDKVIRDVKVITLKSNLVSQFRKDFGFYKVREINDYHHAHDAYLNAVVGTAL |
| | LKKYPKLAPEFVYGEYKKYDVRKIVRSKDESSLGKATAKMFFYSNLMNFFKKEVRLSDGTVIVRP |
| | VIEYSKDTGEIAWNKEKDFATVRKVLSCPQVNIVKKTEVQTGGFSKESILPKRNSDKLIPRKKNWD |
| | PKKYGGFDSPTVAYSVLVVAKVEKGKSKKLKSVKELVGITIMERSSFEKDPIDFLEAKGYKEVRKD |
| | LIIKLPKYSLFELENGRKRMLASAGELQKGNELALPSKYVNFLYLASHYEKLKGSPEDNEQKQLFV |
| | EQHKHYLDEIIEQISEFSERVILADANLDKVLSAYNKHRDKPIREQAENIIHLFTLTNLGAPAAFKFF |
| | DTTIDRKRYTSTKEVLDATLIHQSITGLYETRIDLSKLGGD |
| 297 | MNKPYSIGLDIGTNSVGWSVVDDDYKVPAKKMRVFGDTDKGHIKKNMLGTLLFDSGETAESRRL |
| | KRTARRRYTRRRNRILYLQEIFAKEMAKVDESFFHRLDESFLTDDDKTFDSHPIFGNKAEEDAYHQ |
| | QFPTIYHLRKHLADSTEKADLRLVYLALAHMIKFRGHFLIEGELNAENTDVQKLFADFVGVYDRTF |
| | DDSHLSEITVDASSILTEKISKSRRLEKLIKHYPTEKKNTLFGNLVALALGLQPNFKTNFKLSEDAKL |
| | QFSKDTYEEDLEELLGKIGDDYADLFTSAKNLYDAILLSGILTVDDESTKAPLSASMIKRYAEHHED |
| | LEKLKEFVKANKPELYHDIFKDETKNGYAGYIENGVKQDEFYKYLKSTLSKQDGSQYFLDKIERED |
| | FLRKQRTFDNGSIPHQLHLKEMRSILHRQGEYYPFLKENQAKIEKILTFRIPYYVGPLARKDSRFAW |
| | AEYHSDEPITPWNFDEVVDKEKSAEKFITRMTLNDLYLPEEKVLPKHSHVYETYTVYNELTKVKY |
| | VNEQGEAKFFDANMKQEIFDHVFKENRKVTKDKLMNFLGKEFDEFRIVDITGLDKENKAFNASLG |
| | TYHDLKKILDKSFLDDKENETIIEDIIQTLTLFEDKDMIRQRLQKYSDIFTPQQLKKLERRHYTGWGR |
| | LSYKLINGIRDKQTGKTILGYLIDDGYANRNFMQLINDDTLPFKEIIQKAQIIGDIDDIKAVVHDLPGS |
| | PAIKKGILQSVKIVDELVKVMGHNPDNIVIEMARENQTTNRGRTQSQQRLKKLQESLKELGSNILNE |
| | KKPSYVEDKVENSHLQNDRLFLYYIQNGKDMYTGDELDIDHLSDYDIDHIIPQAFIKDDSIDNRVLT |
| | SSAKNRGKSDDVPSIEIVRDRKADWMRLRKAGLISKRKFDNLTKAERGGLTEADKAGFIKRQLVET |
| | RQITKHVAQILDSRENTERDENDKVIRDVKVITLKSNLVSQFRKDFQFYKVREINDYHHAHDAYLN |
| | AVVGTALIKKYPKLESEFVYGDYKVYDVRKMIAKSEQEIGKATAKRFFYSNIMNFFKTEIKLANGE |
| | IRKRPLIETNEETGEIVWNKEKDFATVRKVLSCPQVNIVKKTEVQTGGFSKESILPKRNSDKLIPRKK |
| | NWDPKKYGGFDSPTVAYSVLVVAKVEKGKSKKLKSVKELVGITIMERSSFEKNPVDFLEAKGYKE |
| | VRKDLIIKLPKYSLFELENGRKRLLASAGELQKGNELALPSKYVNFLYLASHYEKLKGSPEDNEQK |
| | QLFVEQHKYYLDELIDQISDFSKRVILADANLDKLLSAYNNHRDKPIREQAENIIHLFTLTNLGAPA |
| | AFKFFDTTIDRKRYTSTKEILDATLIHQSVTGLYETRIDLSQLGGD |
| 298 | MKKPYSIGLDIGTNSVGWAVVDDDYKVPAKKMRVFGDTDRSHIKKNLLGTLLFDDGNTAESRRL |
| | KRTARRRYTRRRNRILYLQEIFAEEMNKVDESFFHRLDDSFLVPEDKRGSKYPIFATLEEEKEYHKK |
| | FPTIYHLRKHLADSKEKTDLRLIYLALAHMIKYRGHFLYEESFDIKNNDIQKIFNEFISLYDNTFEGSS |
| | LSGQNAQVEAIFTDKISKSAKRERVLKLFPDEKSTGLFSEFLKLIVGNQADFKKHFDLEEKAPLQFS |
| | KDTYDEDLENLLGQIGDDFADLFLVAKKLYDAILLSGILTVTDPSTKAPLSASMIERYENHEEDLAA |
| | LKQFVKANLPEKYDEVFSDQSKDGYAGYIDGKTTQEAFYKYIKNLLSKLEGADYFLDKIEREDFLR |
| | KQRTFDNGSIPHQIHLQEMNAILRRQGEHYPFLKDNKEKIEKILTFRIPYYVGPLARGNRDFAWLTR |
| | NSDQAIRPWNFEEVVDKASSAEDFINKMTNYDLYLPEEKVLPKHSLLYETFAVYNELTKVKFIAEG |
| | LRDYQFLDSGQKKQIVNQLFKTKRKVTEKDIINYLHKVDGYDGVEISGVEDRFNASLGTYHDLLKI |
| | IKDKSFLDDKKNEEIIEDIVLTLTLFEDRDMIRQRLQKYSDIFTPQQLKKLERRHYTGWGRLSYKLIN |
| | GIRNKENNKSILDYLIDDGYANRNFMQLINDDTLPFKQIIQKSQVVGDIDDIKTVVHDLPGSPAIKK |
| | GILQSVKIVDELVKVMGHNPDNIVIEMARENQTTNRGRSQSQQRLKKLQESLKELGSNILNVEKPS |
| | YIEDKVENNHLQNDKLFLYYIQNGKDMYTGEELDIDHLSDYDIDHIIPQAFIKDDSIDNRVLTSSAK |
| | NRGKSDDVPSLDIVRGRKAEWIRLYKAKLISQRKFDNLTKAERGGLTENDKAGFIKRQLVETRQIT |
| | KHVAQILDARFNTKRDENDKLIREVKVITLKSNLVSQFRKEFGFYKVREINDYHHAHDAYLNAVV |
| | GTAILKKYPKLTSEFVYGEYKKYDIRKFIKSDDHSEMGKATAKYFFYSNLLNFFKEEIKYANGKIVV |
| | RPVVEYSKDTGEIAWNKEKDFATVRKVLSCPQVNIVKKTEVQTGGFSKESILPKRNSDKLIARKKD |
| | WDPKKYGGFDSPTVAYSVLVVAKVEKGKSKKLKSVKELLGITIMERSSFEKNPIDFLEAKGYKEVR |
| | KDLIIKLPKYSLFELENGRKRMLASAGELQKGNELALPSKYVNFLYLASHYEKLKGSPEDNEQKQL |
| | FVEQHKHYLDEIIEQISEFSKRVILADANLDKVLSAYNKHRDKPIREQAENIIHLFTLTNLGAPAAFK |
| | YFDTTIDRKRYTSTKEVLDATLIHQSITGLYETRIDLSQLGGD |
| 299 | MKKPYSIGLDIGTNSVGWAVVDDDYKVPAKKMRVFGDTDRSHIKKNLLGTLLFDDGNTAESRRL |
| | KRTARRRYTRRRNRILYLQEIFAEEMNKVDESFFHRLDDSFLVPEDKRGSKYPIFATLEEEKEYHKK |
| | FPTIYHLRKHLADSKEKTDLRLIYLALAHMIKYRGHFLYEESFDIKNNDIQKIFNEFISLYDNTFEGSS |
| | LSGQNAQVEAIFTDKISKSAKRERVLKLFPDEKSTGLFSEFLKLIVGNQADFKKHFDLEEKAPLQFS |
| | KDTYDEDLENLLGQIGDDFADLFLVAKKLYDAILLSGILTVTDPSTKAPLSASMIERYENHEEDLAA |
| | LKQFVKANLPEKYDEVFSDQSKDGYAGYIDGKTTQEAFYKYIKNLLSKLEGADYFLDKIEREDFLR |
| | KQRTFDNGSIPHQIHLQEMNAILRRQGEHYPFLKDNKEKIEKILTFRIPYYVGPLARGNRDFAWLTR |
| | NSDQAIRPWNFEEVVDKASSAEDFINKMTNYDLYLPEEKVLPKHSLLYETFAVYNELTKVKFIAEG |
| | LRDYQFLDSGQKKQIVNQLFKTKRKVTEKDIINYLHKVDGYDGVEISGVEDRFNASLGTYHDLLKI |
| | IKDKSFLDDKKNEEIIEDIVLTLTLFEDRDMIRQRLQKYSDIFTPQQLKKLERRHYTGWGRLSYKLIN |
| | GIRNKENNKSILDYLIDDGYANRNFMQLINDDTLPFKQIIQKSQVVGDIDDIKTVVHDLPGSPAIKK |
| | GILQSVKIVDELVKVMGHNPDNIVIEMARENQTTNRGRSQSQQRLKKLQESLKELGSNILNVEKPS |
| | YIEDKVENNHLQNDKLFLYYIQNGKDMYTGEELDIDHLSDYDIDHIIPQAFIKDDSIDNRVLTSSAK |
| | NRGKSDDVPSLDIVRGRKAEWIRLYKAKLISQRKFDNLTKAERGGLTENDKAGFIKRQLVETRQIT |
| | KHVAQILDARFNTKRDENDKLIREVKVITLKSNLVSQFRKEFGFYKVREINDYHHAHDAYLNAVV |
| | GTAILKKYPKLTSEFVYGEYKKYDIRKFIKSDDHSEMGKATAKYFFYSNLLNFFKEEIKYANGKIVV |
| | RPVVEYSKDTGEIAWNKEKDFATVRKVLSCPQVNIVKKTEVQTGGFSKESILPKRNSDKLIPRKKG |
| | WDPKKYGGFDSPTVAYSVLVVAKVEKGKSKKLKSVKELVGITIMERSSFEKDPVDFLEAKGYKEV |
| | RKDLIIKLPKYSLFELENGRKRMLASAGELQKGNELALPSKYVNFLYLASHYEKLKGSPEDNEQKQ |
| | LFVEQHKHYLDEIVEQISEFSERVILADANLDKVLSAYNKNRDRPIREQAENIIHLFTLTNLGAPAAF |
| | KFFNTDIDRKRYTSTKEILDATLIHQSITGLYETRIDLSQLGGD |
| 300 | MKKPYSIGLDIGTNSVGWAVVTDDYKVPAKKMKVLGDTDKSHIKKNLLGTLLFESGETAEGRRLK |
| | RTARRRYTRRRNRILYLQEIFAEEMNKVDDSFFQRLDESFLKDDDKNFDSHPIFGNKAEEDAYHQK |
| | FPTIYHLRQYLADSPEKADLRLVYLALAHIIKYRGHELFEGDLKSENKDVQHLFNDFVEMEDKTVE |
| | GSYLSENLPNVADVLVEKVSKSRRLENVLHYFPNEKKNGLFGNFIALALGLQPNFKTNFKLSEDAK |
| | LQFSKDTYDEDLEELLGKIGDDYADLFVAAKNLYDAILLSGILTVTDPTTKAPLSASMIDRYEKHH |
| | KDLALLKNLIKQNLTQEKYDEVFSDESKNGYAGYISGKVKQEEFYKYLKNTLSKIEGSDYFLDKIE |
| | REDFLRKQRTFDNGSIPHQIHLQEMNAIIRRQGEHYPFLKENKEKIEKILTFRIPYYVGPLARGNRDF |
| | AWLTRNSDQAIRPWNFEEIVDQASSAEDFINKMTNYDLYLPEEKVLPKHSLLYETFAVYNELTKVK |
| | FIAEGLRDYQFLDSGQKKQIVNQLFKEKRKVTEKDIIQYLHNVDGYDGIELKGIEKQFNASLSTYHD |
| | LLKIIKDKEFMDDPKNEEILENIVHTLTIFEDREMIKQRLAQYDSLFDEKVIKALTRRHYTGWGKLS |
| | AKLINGIYDKQSKKTILDYLIDDGEINRNFMQLINDDGLSFKDIIQKAQVVGKTNDVKQVVQELPGS |
| | PAIKKGILQSIKLVDELVKVMGHAPESIVIEMARENQTTARGKKNSQQRYKRIEDALKNLAPGLDS |
| | NILKENPTDNIQLQNDRLFLYYLQNGKDMYTGEALDINQLSNYDIDHIVPQAFIKDDSLDNRVLTSS |
| | KDNRGKSDNVPSIEVVQKRKAFWQQLLDSKLISERKENNLTKAERGGLDERDKVGFIKRQLVETR |
| | QITKHVAQILDARFNTEVDENNQRIRKVKIITLKSNLVSNFRKEFGLYKVREINDYHHAHDAYLNA |
| | VVAKAILKKYPKLEPEFVYGDYQKYDLKRYISRSKDPKDIEKATEKYFFYSNLLNFFKEEVHYADG |
| | TIIKRENIEYSNDTGEIAWNKEKDFATVRKVLSCPQVNIVKKTEEQTGGFSKESILPKRNSDKLIPRK |
| | TEWDPKKYGGFDSPIVAYSVLVVAKVEKGKSKKLKSVKELVGITIMERSSFEKDPIDFLEAKGYKE |
| | VRKDLIIKLPKYSLFELENGRKRMLASAGELQKGNELALPSKYVNFLYLASHYEKLKGSPEDNEQK |
| | QLFVEQHKHYLDEIVEQISEFSERVILADANLDKVLSAYNKHRDKPIREQAENIIHLFTLTNLGAPAA |
| | FKFFNTDIDRKRYTSTKEVLDATLIHQSITGLYETRIDLSQLGGD |
| 301 | MKKPYSIGLDIGTNSVGWAVVTDDYKVPAKKMKVLGDTDKSHIKKNLLGTLLFESGETAEGRRLK |
| | RTARRRYTRRRNRILYLQEIFAEEMNKVDDSFFQRLDESFLKDDDKNFDSHPIFGNKAEEDAYHQK |
| | FPTIYHLRQYLADSPEKADLRLVYLALAHIIKYRGHELFEGDLKSENKDVQHLFNDFVEMEDKTVE |
| | GSYLSENLPNVADVLVEKVSKSRRLENVLHYFPNEKKNGLFGNFIALALGLQPNFKTNFKLSEDAK |
| | LQFSKDTYDEDLEELLGKIGDDYADLFVAAKNLYDAILLSGILTVTDPTTKAPLSASMIDRYEKHH |
| | KDLALLKNLIKQNLTQEKYDEVFSDESKNGYAGYISGKVKQEEFYKYLKNTLSKIEGSDYFLDKIE |
| | REDFLRKQRTFDNGSIPHQIHLQEMNAIIRRQGEHYPFLKENKEKIEKILTFRIPYYVGPLARGNRDF |
| | AWLTRNSDQAIRPWNFEEIVDQASSAEDFINKMTNYDLYLPEEKVLPKHSLLYETFAVYNELTKVK |
| | FIAEGLRDYQFLDSGQKKQIVNQLFKEKRKVTEKDIIQYLHNVDGYDGIELKGIEKQFNASLSTYHD |
| | LLKIIKDKEFMDDPKNEEILENIVHTLTIFEDREMIKQRLAQYDSLFDEKVIKALTRRHYTGWGKLS |
| | AKLINGIYDKQSKKTILDYLIDDGEINRNFMQLINDDGLSFKDIIQKAQVVGKTNDVKQVVQELPGS |
| | PAIKKGILQSIKLVDELVKVMGHAPESIVIEMARENQTTARGKKNSQQRYKRIEDALKNLAPGLDS |
| | NILKENPTDNIQLQNDRLFLYYLQNGKDMYTGEALDINQLSNYDIDHIVPQAFIKDDSLDNRVLTSS |
| | KDNRGKSDNVPSIEVVQKRKAFWQQLLDSKLISERKENNLTKAERGGLDERDKVGFIKRQLVETR |
| | QITKHVAQILDARFNTEVDENNQRIRKVKIITLKSNLVSNFRKEFGLYKVREINDYHHAHDAYLNA |
| | VVAKAILKKYPKLEPEFVYGDYQKYDLKRYISRSKDPKDIEKATEKYFFYSNLLNFFKEEVHYADG |
| | TIIKRENIEYSNDTGEIAWNKEKDFATVRKVLSCPQVNIVKKTEVQTGGFSKESILPKRNSDKLIPRK |
| | KGWDPKKYGGFDSPTVAYSVLVVAKVEKGKSKKLKSVKELVGITIMERSSFEKDPVDFLEAKGYK |
| | EVRKDLIIKLPKYSLFELENGRKRMLASAGELQKGNELALPSKYVNFLYLASHYEKLKGSPEDNEQ |
| | KQLFVEQHKHYLDEIVEQISEFSERVILADANLDKVLSAYNKNRDRPIREQAENIIHLFTLTNLGAPA |
| | AFKFFNTDIDRKRYTSTKEILDATLIHQSITGLYETRIDLSQLGGD |
| 302 | MKKSYSIGLDIGTNSVGWAVVDDDYKVPAKKMRVFGDTDRSHIKKNLLGTLLFDEGNTAESRRL |
| | KRTARRRYTRRRNRILYLQEIFAEEMNKVDESFFHRLDDSFLVPEDKRGSKYPIFATLQEEKEYHKQ |
| | FPTIYHLRKQLAESNEKADLRLVYLALAHMIKYRGHFLIDDPKFKVQNNDIQGLFEKFVEEYDNVQ |
| | ETSLSKIKLNVTEILTANIPKSEKQEQLLKNYPSEKKNTLFGNLIGLALGLTPNFKTNFSLENDAKLQI |
| | SSESYEEDLGSLLALIGENFIELFSAVKNLSDGILLAGIVSDESPHAPLSTKMVIRFKEHEEDLAALKH |
| | FIKTNLPEKYDEVFSDDSKNGYAGYVGVDSKVRKRNGKLATEEEFYKYLKEILNNVKGADYFLEK |
| | IKREDLLRKQRTFDNGTIPYQVHLTEMKAIIHRQGEHYPFLQENKEKIEKILTFRIPYYVGPLARGNR |
| | DFAWLTRNSDQAIRPWNFEEIVDQASSAEDFINKMTNYDLYLPEEKVLPKHSLLYETFAVYNELTK |
| | VKFIAEGLRDYQFLDSGQKKQIVNQLFKEKRKVTEKDIIHYLHNVDGYDGIELKGIEKQFNASLSTY |
| | HDLLKIIKDKEFMDDPKNEEILENIVHTLTIFEDREMIKQRLAQYDSLFDEKVIKALTRRHYTGWGK |
| | LSAKLINGICDKKTGKTILDYLIDDDKINRNFMQLINDDGLSFKEIIQKAQVVGKTDDVKQVVQELP |
| | GSPAIKKGILQSIKIVDELVKVMGHAPESIVIEMARENQTTARGKKNSQQRYKRIEDALKNLAPGLD |
| | SNILKENPTDNIQLQNDRLFLYYLQNGKDMYTGEALDINQLSSYDIDHIVPQAFIKDDSLDNRVLTS |
| | SKDNRGKSDNVPSIEVVQKRKAFWQQLLDSKLISERKENNLTKAERGGLDERDKVGFIKRQLVETR |
| | QITKHVAQILDARFNTEVNEKGQKIRKVKIITLKSNLVSNFRKEFGLYKVREINDYHHAHDAYLNA |
| | VVAKAILKKYPKLEPEFVYGDYQKYDIKRYISRSKDPKEIEKATEKYFFYSNLLNFFKEEVHYADG |
| | TIVKRENIEYSKDTGEIAWNKEKDFATVRKVLSCPQVNIVKKTEVQTGGFSKESILPKRNSDKLIAR |
| | KKDWDPKKYGGFDSPTVAYSVLVVAKVEKGKSKKLKSVKELLGITIMERSSFEKNPIDFLEAKGY |
| | KEVRKDLIIKLPKYSLFELENGRKRMLASAGELQKGNELALPSKYVNFLYLASHYEKLKGSPEDNE |
| | QKQLFVEQHKHYLDEIIEQISEFSKRVILADANLDKVLSAYNKHRDKPIREQAENIIHLFTLTNLGAP |
| | AAFKYFDTTIDRKRYTSTKEVLDATLIHQSITGLYETRIDLSQLGGD |
| 303 | MKKPYSIGLDIGTNSVGWAVVTDDYKVPAKKMKVLGNTDKSHIKKNLLGALLFDSGETAEGRRL |
| | KRTARRRYTRRRNRILYLQEIFAEEMNKIDESFFHRLDDSFLVPEDKRGSKYPIFATLEEEKEYHKK |
| | FPTIYHLRKHLAESNEKADLRLVYLALAHMIKYRGHFLIDDPKFKVQNNDIQGLFEKFVEEYDNVQ |
| | ETSLSKIKLNVTEILTANIPKSEKQEQLLKNYPSEKKNTLFGNLIGLALGLTPNFKTNFSLENDAKLQI |
| | SSESYEEDLGNLLALIGENFIELFSAVKNLSDGILLAGIVSDESPHAPLSTKMVIRFKEHEEDLAALK |
| | HFIKANLPEKYDEVFSDDSKNGYAGYVGVDSKVRKRNGKLATEEEFYKYLKDILNNVKGADYFLE |
| | KIKREDLLRKQRTFDNGTIPYQVHLSEMKAIIHRQGEHYPFLKENKEKIEKILTFRIPYYVGPLARGN |
| | RDFAWLTRNSDQAIRPWNFEEIVDKASSAEDFINKMTNYDLYLPEEKVLPKHSLLYETFAVYNELT |
| | KVKFIAEGLRDYQFLDSGQKKQIVNQLFKEKRKVTEKDIIHYLHNVDGYDGIELKGIEKQFNASLST |
| | YHDLLKIIKDKEFMDDPKNEEILENIVHTLTIFEDREMIKQRLAQYDSLFDEKVIKALTRRHYTGWG |
| | KLSAKLINGIYDKQSKKTILDYLIDDGEINRNFMQLIHDDGLSFKEIIQKAQVFGKTDDVKQVVQEL |
| | PGSPAIKKGILQSIKIVDELVKVMGHAPESIVIEMARENQTTARGKKNSQQRYKRIEDSLKNLASGL |
| | DSNILKENPTDNVQLQNDRLFLYYLQNGKDMYTGEALDINQLSNYDIDHIIPQAFIKDDSLDNRVLT |
| | SSKDNRGKSDNVPSIEVVEKMKAFWQQLLDSKLISERKYNNLTKAERERGGLNELDKVGFIKRQL |
| | VETRQITKHVAQILDARFNTEVNENNQKIRKVKIITLKSNLVSNFRKEFGLYKVREINDYHHAHDA |
| | YLNAVVAKAILKKYPKLEPEFVYGDYQKYDIKRYISRSKDPKDIEKATEKYFFYSNLLNFFKEEVH |
| | YADGTIIKRENIEYSKDTGEIAWNKEKDFATVRKVLSCPQVNIVKKTEVQTGGFSKESILPKRNSDK |
| | LIARKKDWDPKKYGGFDSPTVAYSVLVVAKVEKGKSKKLKSVKELVGITIMERSSFEKDPVDFLE |
| | AKGYKEVRKDLIIKLPKYSLFELENGRKRMLASAGELQKGNELALPSKYVNFLYLASHYEKLKGSP |
| | EDNEQKQLFVEQHKHYLDEIIEQISEFSKRVILADANLDKVLSAYNKHRDKPIREQAENIIHLFTLTN |
| | LGAPAAFKYFDTTIDRKRYTSTKEVLDATLIHQSITGLYETRIDLSQLGGD |
| 304 | MKKPYSIGLDIGTNSVGWAVVTDDYKVPAKKMKVLGNTDKSHIKKNLLGTLLFDSGETAEGRRL |
| | KRTARRRYTRRRNRILYLQEIFAEEMNKVDESFFHRLDDSFLVPEDKRGSKYPIFGTLEEEKEYHKQ |
| | FPTIYHLRKQLAESNEKADLRLVYLALAHMIKYRGHFLIDDPKFKVQNNDIQGLFEKFVEEYDNVQ |
| | ETSLSKIKLNVTEILTANIPKSEKQEQLLKNYPSEKKNTLFGNLIGLALGLTPNFKTNFSLENDAKLQI |
| | SSESYEEDLGNLLALIGENFIELFSAVKNLSDGILLAGIVSDETPHAPLSTKMVIRFKEHEEDLAALK |
| | HFIKANLPEKYDEVFSDDSKNGYAGYVGVDSKVRKRNGKLATEEEFYKYLKEILNNVKGADYFLE |
| | KIKREDLLRKQRTFDNGTIPYQVHLSEMKAIIHRQGEHYPFLQENKEKIEKILTFRIPYYVGPLARGN |
| | RDFAWLTRNSDQAIRPWNFEEIVDQASSAEDFINKMTNYDLYLPEEKVLPKHSLLYETFAVYNELT |
| | KVKFIAEGLRDYQFLDSGQKKQIVNQLFKEKRKVTEKDIIHYLHNVDGYDGIELKGIEKQFNASLST |
| | YHDLLKIIKDKEFMDDPKNEEILENIVHTLTIFEDREMIKQRLAQYDSLFDEKVIKALTRRHYTGWG |
| | KLSAKLINGIRDKQSNKTILDYLIDDDKINRNFMQLINDDGLSFKEIIQKAQVVGKTDDVKQVVQEL |
| | PGSPAIKKGILQSIKIVDELVKVMGHAPESIVIEMARENQTTARGKKNSQQRYKRIEDALKNLAPGL |
| | DSNILKENPTDNIQLQNDRLFLYYLQNGKDMYTGNPLDINQLSSYDIDHIVPQAFIKDDSLDNRVLT |
| | SSKDNRGKSDNVPSIEVVQKRKAFWQQLLDSKLISERKENNLTKAERGGLDERDKVGFIKRQLVET |
| | RQITKHVAQILDARFNTEVNEKNQKIRSVKIITLKSNLVSNFRKEFRLYKVREINDYHHAHDAYLNA |
| | VVAKAILKKYPKLEPEFVYGDYQKYDIKRYISRSKDPKDIEKATEKYFFYSNLLNFFKEEVHYADG |
| | TIIKRENIEYSKDTGEIAWNKEKDFATVRKVLSCPQVNIVKKTEVQTGGFSKESILPKRNSDKLIARK |
| | KDWDPKKYGGFDSPTVAYSVLVVAKVEKGKSKKLKSVKELVGITIMERSSFEKDPVDFLEAKGYK |
| | EVRKDLIIKLPKYSLFELENGRKRMLASAGELQKGNELALPSKYVNFLYLASHYEKLKGSPEDNEQ |
| | KQLFVEQHKHYLDEIIEQISEFSKRVILADANLDKVLSAYNKHRDKPIREQAENIIHLFTLTNLGAPA |
| | AFKYFDTTIDRKRYTSTKEVLDATLIHQSITGLYETRIDLSQLGGD |

| SEQ ID | |
|---|---|
| NO | gRNA Scaffold Sequence (shown as DNA) |
| 305 | gttttagagctgtgttgaaaaacacagcaaagttaaaataaggctttgtccgt |
| | atccaacttgaaaaagtgcgcaccgattcggtgcttttttgttttacta |
| 306 | gttttagagctgtgttgaaaaacacagcaagttaaaataaggctttgtccgta |
| | tccaacttgaaaaagtgagcaccgattcggtgctttttttgtgccca |
| 307 | gttttagagctgtgttgaaaaacacagcaagttaaaataaggctttgtccgta |
| | cacaacttgaaaaagtgagcaccgattcggtgctttttttgtgccta |
| 308 | gttttagagctgtgttgaaaaacacagcaagttaaaataaggctttgtccgta |
| | cacaacttgaaaaagtgagcaccgattcggtgctttttttgtgccca |
| 309 | gttttagagctgtgttgaaaaacacagcaagttaaaataaggctttgtccgta |
| | cacaacttgaaaaagtgagcaccgattcggtgcttttttgttttcta |
| 310 | gttttagagctgtgttgaaaaacacagcaagttaaaataaggctttgtccgta |
| | cacaacttgaaaaagtgcgcaccgattcggtgctttttttgtgcctt |
| 311 | gttttagagctgtgttgaaaaacacagcaagttaaaataaggctttgtccgta |
| | cacaacttgaaaaagtgagcaccgattcggtgcttttttgtatacaaa |
| 312 | gttttagagctgtgttgaaaaacacagcaaagttaaaataaggctttgtccgt |
| | acacaacttgaaaaagtgcgcaccgattcggtgcttttttgttttacta |
| 313 | gttttagagctgtgttgaaaaacacagcaagttaaaataaggctttgtccgta |
| | tccaacttgaaaaagtgcgcaccgattcggtgctttttttgtgccc |
| 314 | gttttagagctgtgttgaaaaacacagcaagttaaaataaggcttagtccgta |
| | ttcaacttgaaaaagtgcgcaccgattcggtgcttttttgttttacta |
| 315 | gttttagagctgtgttgaaaaacacagcaagttaaaataaggcttagtccgta |
| | cacaacttgaaaaagtgcgcaccgattcggtgctttttttgtgccc |
| 316 | gttttagagctgtgttgaaaaacacagcaagttaaaataaggctttgtccgta |
| | cacaacttgaaaaagtgagcaccgattcggtgcttttttgttttacta |
| 317 | gttttagagctgtgttgaaaaacacagcaagttaaaataaggctttgtccgt |
| | acacaacttgaaaaagtgagcaccgattcggtgcttttttgttttacacaac |
| 318 | gttttagagctgtgttgaaaaacacagcaagttaaaataaggctttgtccgta |
| | cacaacttgaaaaagtgagcaccgattcggtgcttttttattttata |
| 319 | gttttagagctgtgttgaaaaacacagcaagttaaaataaggctttgtccgta |
| | cacaacttgaaaaagtgcgcaccgattcggtgctttttttgtgccc |
| 320 | gttttagagctgtgttgaaaaacacagcaagttaaaataaggctttgtccgta |
| | cacaacttgaaaaagtgagcaccgattcggtgctttttttgtgcccta |
| 321 | gttttagagctgtgctgaaaaacacagcgagttaaaataaggctttgtccgta |
| | cacaacttgaaaaagtggcaccgattcggtgcttttttattttatt |
| 322 | gttttagagctgtgctgaaaaacacagcaagttaaaataaggctttgtccgta |
| | cacaacttgtaaaagtgagcaccgattcggtgcttttttgttgctt |
| 323 | gttttagagctgtgttgaaaaacacagcaagttaaaataaggctttgtccgta |
| | cacaacttgaaaaagtgagcaccgattcggtgctttttttgtataca |
| 324 | gttttagagctgtgctgaaaaacacagcgagttaaaataaggctttgtccgta |
| | cacaacttgtaaaagtgagcaccgattcggtgcttttttatttgct |
| 325 | gttttagagctgtgctgaaaaacacagcgagttaaaataaggctttgtccgta |
| | cacaacttgaaaaagtgagcaccgattcggtgctttttttttt |
| 326 | gttttagagctgtgctgaaaaacacagcgagttaaaataaggctttgtccgta |
| | cacaacttgaaaaagtgcgcaccgattcggtgcttttttgttatttt |
| 327 | gttttagagctgtgctgaaaaacacagcaagttaaaataaggctttgtccgta |
| | cacaacttgaaaaagtggcaccgattcggtgcttttttgttttatgccc |
| 328 | gttttagagctgtgttgaaaaacacagcgagttaaaataaggctttgtccgta |
| | cacaacttgaaaaagtgagcaccgattcggtgcttttttgttttatgcctca |
| 329 | gttttagagctgtgctgaaaaacacagtgagttaaaataaggctttgtccgta |
| | cacaacttgaaaaagtgagcaccgattcggtgcttttttgttgctca |
| 330 | gttttagagctgtgctgaaaaacacagcgagttaaaataaggctttgtccgta |
| | cacacaacttgaaaaagtgcgcaccgattcggtgcttttttgttttctt |
| 331 | gttttagagctgtgctgaaaaacacagcgagttaaaataaggtttagtccgta |
| | cacaacttgaaaaagtgagcaccgattcggtgcttttttgttttctt |
| 332 | gttttagagctgtgctgaaaaacacagcgagttaaaataaggctttgtccgta |
| | cacaacttgtttaagtgcgcaccgattcggtgcttttttgttttata |
| 333 | gttttagagctgtgctgaaaaacacagttagttaaaataaggctttgtccgta |
| | cacaacttgaaaaagtgagcaccgattcggtgcttttttgttttctt |
| 334 | gttttagagctgtgttgaaaaacatagcaagttaaaataaggctttgtccgta |
| | cacaacttgaaaaagtgcgcaccgattcggtgcttttttgttttacta |
| 335 | gttttagagctgtgctgaaaaacacagcgagttaaaataaggctttgtccgta |
| | cacaacttttgaaaaagtgcgcaccgattcggtgcttttttgttttatt |
| 336 | gttttagagctgtgctgaaaaacacagcgagttaaaataaggctttgtccgta |
| | cacaacttgaaaaagtgagcaccgattcggtgcttttttattttgact |
| 337 | gttttagagctgtgctgaaaaacacagcgagttaaaataaggctttgtccgta |
| | cacaacttgttaaagtggcacccgattcgggtgcttttttattttct |
| 338 | gttttagagctgtgttgaaaaacacagtgagttaaaataaggctttgtccgta |
| | cacaacttaaaaaagtggcacccgattcgggtgctttttttattttc |
| 339 | gttttagagctgtgctgaaaaacacagcgagttaaaataaggctttgtccgta |
| | cacaacttaaaaaagtggcacccgattcgggtgctttttttattttaaa |
| 340 | gttttagagctgtgctgaaaaacacagcaagttaaaataaggctttgtccgta |
| | cacaacttgtaaaagtggcacccgattcggatgcgttttttatttgtgt |
| 341 | gttttagagctgtgctgaaaaacacagcaagttaaaataaggctttgtccgta |
| | cacaacttaaaaaagtggcacccgattcgggtgcattttttgtgcttt |
| 342 | gttttagagctgtgctgaaaaacacagcaagttaaaataaggctttgtccgta |
| | cacaacttgtaaaagtggcacccgattcgggtgcttttttattttatt |
| 343 | gttttagagctgtgctgaaaaacacagcaagttaaaataaggctttgtccgta |
| | cacaacttaaaaaagtgagcaccgattcggtgcttttttattttct |
| 344 | gttttagagctgtgctgaaaaacacagcaagttaaaataaggctttgtccgta |
| | cacaacttgtgaaagtaagcaccgattcggtgcttttttgttttcta |
| 345 | gttttagagctgtgttgaaaaacacagcaagttaaaataaggctttgtccgta |
| | cacaacttgaaaaagtgagcaccgattcggtgctttttttgtgcctt |
| 346 | gttttagagctgtgttgaaaaacacagcgagttaaaataaggctttgtccgta |
| | cacaacttgtaaaagtggcacccgattcgggtgctttttttatttttct |
| 347 | gttttagagctgtgctgaaaaacacagcgagttaaaataaggctttgtccgta |
| | cacaacttgtaaaagtggcacccgattcgggtgccttttaattttctt |
| 348 | gttttagagctgtgctgaaaaacacagtgagttaaaataaggctttgtccgta |
| | cacaacttgtaaaagtggcacccgattcgggtgcattttttattttct |
| 349 | gttttagagctgtgctgaaaaacacagcgagttaaaataaggctttgtccgta |
| | cacaacttataaaagtgcgcaccgattcggtgcttttttattttatt |
| 350 | gttttagagctgtgctgaaaaacacagcaagttaaaataaggctttgtccgta |
| | cacaacttaaaaaagtggcacccgattcggatgcgttttttgtgtacat |
| 351 | gttttagagctgtgctgaaaaacacagcgagttaaaataaggctttgtccgta |
| | cacaacttgtaaaagtgagcaccgattcggtgcttttttattttttct |
| 352 | gttttagagctgtgttgaaaaacacagcgagttaaaataaggctttgtccgta |
| | cacaacttgtaaaagtgagcaccgattcggtgcttttttattttatt |
| 353 | gttttagagctgtgctgaaaaacacagcgagttaaaataaggctttgtccgta |
| | cacaacttgtaaaagtgagcaccgattcggtgcttttttgttgaca |
| 354 | gttttagagctgtgctgaaaaacacagcaagttaaaataaggctttgtccgta |
| | cacaacttgtaaaagtggcacccgattcgggtgcatttttttgtgtgt |
| 355 | gttttagagctgtgttgaaaaacacagcaagttaaaataaggctttgtccgta |
| | cacaacttgaaaaagtgcgcaccgattcggtgcttttttgttgcgta |
| 356 | gttttagagctgtgttgaaaaacacagcgagttaaaataaggctttgtccgta |
| | cacaacttgtaaaagtggcacccgattcgggtgcattttttattttaaca |
| 357 | gttttagagctgtgctgaaaaacacagtgagttaaaataaggctttgtccgta |
| | cacaacttgtaaaaggtgcgcaccgattcggtgctttttttattttct |
| 358 | gttttagagctgtgttgaaaaacacagcgagttaaaataaggctttgtccgta |
| | cacattttggcaaagtgagcaccgattcggtgcttttttattttatt |
| 359 | gttttagagctgtgctgaaaaacacagcaagttaaaataaggctttgtccgta |
| | cacaacttaaaaaagtggcaccccgattcggtgctttttttattttct |
| 360 | gttttagagctgtgctgaaaaacacagcgagttaaaataaggctagtccgtat |
| | ccaacttgaaaaagtggcaccccgattcggatgcattttttatttgtg |
| 361 | gttttagagctgtgctgaaaaacacagcgagttaaaataaggctttgtccgta |
| | cacaacttgtaaaagtggcacccgattcggtgcttttttatttctt |
| 362 | gttttagagctgtgctgaaaaacacagcgagttaaaataaggctttgtccgta |
| | cacaacttgtaaaagtgagcaccgattcggtgtttttttattttaaca |
| 363 | gttttagagctgtgctgaaaaacacagcgagttaaaataaggctttgtccgta |
| | cacaacttgaaaaagtggcacccgattcgggtgcattttttgtgtgt |
| 364 | gttttagagctgtgctgaaaaacacagcgagttaaaataaggctagtccgtac |
| | acaacttgtgaaagtggcacccgattcgggtgcattttttattttct |
| 365 | gttttagagctgtgttgaaaaacacagcgagttaaaataaggctttgtccgta |
| | cacaacttttgaaaaagtgcgcaccgattcggtgcttttttattttatt |
| 366 | gttttagagctgtgttgaaaaacacagcaagttaaaataaggctttgtccgta |
| | tccaacttgaaaaagtgcgcaccgattcggtgcttttttgttttacta |
| 367 | gttttagagctgtgctgaaaaacacagcaagttaaaataaggctttgtccgta |
| | cacaacttgtaaaagtggcacccgattcgggtgcgttttttattttct |
| 368 | gttttagagctgtgctgaaaaacacagtgagttaaaataaggctttgtccgta |
| | cacaacttgtaaaagtgagcaccgattcggtgctttttttattttact |
| 369 | gttttagagctgtgctgaaaaacacagcaagttaaaataaggctttgtccgta |
| | cacaacttgtaaaagtgagcaccgattcggtgctttttttatttttct |
| 370 | gttttagagctgtgctgaaaaacacagcaagttaaaataaggctttgtccgta |
| | cacaacttaaaaaagtggcacccgattcgggtgcattttttattttct |
| 371 | gttttagagctgtgctgaaaaacacagtgagttaaaataaggctttgtccgta |
| | cacaacttgtgaaagtacgcaccgattcggtgctttttttattttc |
| 372 | gttttagagctgtgctgaaaaacacagcaagttaaaataaggctttgtccgta |
| | cacaacttgtaaaaggagcgccgatgctttttttgtgccc |
| 373 | gttttagagctgtgctgaaaaacacagtgagttaaaataaggctttgtccgta |
| | cacaacttgtaaaagtggcacccaattcgggtgcgttttttattttct |
| 374 | gttttagagctgtgctgaaaaacacagcaagttaaaataaggctttgtccgta |
| | cacaacttgtaaaaggtgcgcaccgattcggtgtttttttattttacca |
| 375 | gttttagagctgtgctgaaaaacacagcaagttaaaataaggctttgtccgta |
| | cacattttggccccccgattcgggtgcgttttttgtgccc |
| 376 | gttttagagctgtgctgaaaaacacagcaagttaaaataaggctttgtccgta |
| | cacaacttgtaaaagtggcacccgattcggatgcattttttattgtgg |
| 377 | gttttagagctgtgttgaaaaacacagcaagttaaaataaggctttgtccgta |
| | cacaacttgaaaaagtgagcaccgattcggtgcttttttgttgcgta |
| 378 | gttttagagctgtgctgaaaaacacagcaagttaaaataaggctttgtccgta |
| | cacaacttgtgaaagtacgcaccgattcggtgctttttttatttac |
| 379 | gttttagagctgtgctgaaaaacacagcaagttaaaataaggctttgtccgta |
| | cacaacttgtaaaagtggcaccccgattcgggtgcgtttttttgtgccc |
| 380 | gttttagagctgtgctgaaaaacacagcaagttaaaataaggctttgtccgta |
| | cacaacttaaaaaagtggcgccccgattcggtgctttttttattgtgt |
| 381 | gttttagagctgtgctgaaaaacacagcaagttaaaataaggctttgtccgta |
| | cacaacttgtaaaagtggcacccaattcgggtgcgtttttttatttctct |
| 382 | gttttagagctgtgttgaaaaacacagcaagttaaaataaggctttgtccgta |
| | cacaacttgaaaaagtgagcaccgattcggtgctttttattttatta |
| 412 | gttttagagctaGAAAtagcaagttaaaataaggctagtccgttatcaacttg |
| | aaaaagtggcaccgagtcggtgc |

The scope of the present invention is not limited by what has been specifically shown and described hereinabove. Those skilled in the art will recognize that there are suitable alternatives to the depicted examples of materials, configurations, constructions, and dimensions. Variations, modifications, and other implementations of what is described herein will occur to those of ordinary skill in the art without departing from the spirit and scope of the invention.

Numerous references, including patents and various publications, are cited and discussed in the description of this invention. The citation and discussion of such references is provided merely to clarify the description of the present invention and is not an admission that any reference is prior art to the invention described herein. All references cited and discussed in this specification are incorporated herein by reference in their entirety.

The invention claimed is:

1. An engineered nuclease having an amino acid sequence with at least 75% identity to any one of SEQ ID NOs: 240, 280, 214, 229, 303, and 204.

2. The engineered nuclease of claim 1, wherein the engineered nuclease has an amino acid sequence with at least 90% identity to any one of SEQ ID NOs: 240, 280, 214, 229, 303, and 204.

3. The engineered nuclease of claim 1, wherein the engineered nuclease has an amino acid sequence of any one of SEQ ID NO: 240, 280, 214, 229, 303, and 204.

4. The engineered nuclease of claim 1, comprising one or more amino acid substitutions configured to fully or partially catalytically inactivate the engineered nuclease.

5. The engineered nuclease of claim 1, wherein the engineered nuclease further comprises a localization sequence, a tag sequence, a protein transduction domain sequence, or a combination thereof.

6. A fusion protein comprising an engineered nuclease of claim 1 and one or more effector domains.

7. The fusion protein of claim 6, wherein the one or more effector domains are each individually selected from the group consisting of: a transcription activator, a transcription repressor, a deaminase, a polymerase, an epigenetic modifier, and a detection agent.

8. The fusion protein of claim 6, comprising one or more amino acid substitutions configured to fully or partially catalytically inactivate the engineered nuclease.

9. A nucleic acid encoding the engineered nuclease of claim 1 or a fusion protein thereof.

10. A system comprising an engineered nuclease of claim 1, and/or a fusion protein thereof, and at least one guide RNA (gRNA), or one or more nucleic acids encoding thereof.

11. The system of claim 10, wherein the at least one gRNA is complexed with the nuclease.

12. A cell comprising an engineered nuclease of claim 1 or a fusion protein thereof.

13. The cell of claim 12, wherein the cell is a prokaryotic cell or a eukaryotic cell.

14. A method of modifying a target nucleic acid comprising contacting the target nucleic acid with: an engineered nuclease of claim 1 and/or a fusion protein thereof; and at least one guide RNA.

15. The method of claim 14, wherein the target nucleic acid is associated with a disease or disorder.

16. The method of claim 14, wherein the target nucleic acid encodes a gene product.

17. The method of claim 16, wherein the target nucleic acid is a disease-associated gene.

18. The method of claim 14, wherein the target nucleic acid is in a cell and the contacting comprises introducing the engineered nuclease, and/or a fusion protein thereof, and at least one guide RNA, or one or more nucleic acids encoding the engineered nuclease, and/or a fusion protein thereof, and the at least one guide RNA into the cell.

19. The method of claim 18, wherein the cell is in vivo, in vitro, or ex vivo.

20. The method of claim 19, wherein the introducing comprises administering to a subject.
